SEQUENCE LISTING (10 sequences)

SEQ ID NO: 1 - protein, 219 aa, Unknown
  Description of Unknown mutant p53 sequence
SSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSFEVHVCACPGRDRRTEEENLRKKGEPHHELPPGSTKRALSNNT

SEQ ID NO: 2 - DNA, 18 bp, Artificial Sequence
  Description of Artificial Sequence Synthetic primer
ccagctgccctacttgga

SEQ ID NO: 3 - DNA, 20 bp, Artificial Sequence
  Description of Artificial Sequence Synthetic primer
gtgtcccttgttgtgctgtg

SEQ ID NO: 4 - DNA, 8 bp, Artificial Sequence
  Description of Artificial Sequence Synthetic probe
cctggaga

SEQ ID NO: 5 - DNA, 18 bp, Artificial Sequence
  Description of Artificial Sequence Synthetic primer
acttcgggatgggaaagc

SEQ ID NO: 6 - DNA, 18 bp, Artificial Sequence
  Description of Artificial Sequence Synthetic primer
gaggaaggtggggtccat

SEQ ID NO: 7 - DNA, 8 bp, Artificial Sequence
  Description of Artificial Sequence Synthetic probe
tggaggag

SEQ ID NO: 8 - DNA, 21 bp, Artificial Sequence
  Description of Artificial Sequence Synthetic primer
gatggctctcatcccagactt

SEQ ID NO: 9 - DNA, 20 bp, Artificial Sequence
  Description of Artificial Sequence Synthetic primer
agtccatgtgaatgggttcc

SEQ ID NO: 10 - DNA, 8 bp, Artificial Sequence
  Description of Artificial Sequence Synthetic probe
ttctcctg

(19) United States
(12) Patent Application Publication
(10) Pub. No.: US 2023/0049952 A1
(43) Pub. Date: Feb. 16, 2023

(54) METHODS AND COMPOUNDS FOR RESTORING MUTANT P53 FUNCTION

(71) Applicant: PMV Pharmaceuticals, Inc., Cranbury, NJ (US)

(72) Inventors: Arnold Levine, Doylestown, PA (US); David Mack, Menlo Park, CA (US); Binh Vu, North Caldwell, NJ (US); Thomas Davis, South Orange, NJ (US); Melissa Dumble, Watchung, NJ (US)

(21) Appl. No.: 17/348,488

(22) Filed: Jun. 15, 2021

Related U.S. Application Data
(60) Provisional application No. 63/043,535, filed on Jun. 24, 2020.
(60) Provisional application No. 63/162,213, filed on Mar. 17, 2021.

(51) Int. Cl. A61K 31/454; A61K 31/4045; A61P 35/00

(52) U.S. Cl. CPC A61K 31/454; A61P 35/00; A61K 31/4045

(57) ABSTRACT

Mutations in oncogenes and tumor suppressors contribute to the development and progression of cancer. The present disclosure describes compounds and methods to recover wild-type function to p53 mutants. The compounds of the present invention can bind to mutant p53 and restore the ability of the p53 mutant to bind DNA and activate downstream effectors involved in tumor suppression. The disclosed compounds can be used to reduce the progression of cancers that contain a p53 mutation.

### CROSS REFERENCE

This application claims the benefit of U.S. Provisional Application No. 63/043,535, filed Jun. 24, 2020; and U.S. Provisional Application No. 63/162,213, filed Mar. 17, 2021, which are incorporated herein by reference.

### BACKGROUND

Cancer, an uncontrolled proliferation of cells, is a multifactorial disease characterized by tumor formation, growth, and in some instances, metastasis. Cells carrying an activated oncogene, damaged genome, or other cancer-promoting alterations can be prevented from replicating through an elaborate tumor suppression network. A central component of this tumor suppression network is p53, one of the most potent tumor suppressors in the cell. Both the wild type and mutant conformations of p53 are implicated in the progression of cancer.

### INCORPORATION BY REFERENCE

Each patent, publication, and non-patent literature cited in the application is hereby incorporated by reference in its entirety as if each was incorporated by reference individually.

### SUMMARY OF THE INVENTION

Disclosed herein is a method of treating a cancer, the method comprising administering to a subject in need thereof a therapeutically-effective amount of a compound that binds to a mutant p53 protein and reconforms the mutant p53 protein to a conformation of p53 that exhibits anti-cancer activity, wherein the mutant p53 protein comprises a mutation at Y220C, wherein the compound has a half-maximal inhibitory concentration (IC₅₀) in a cancer cell that has a Y220C mutant p53 protein that is at least about 2-fold lesser than in a cancer cell that does not have any Y220C mutant p53 protein.

Disclosed herein is a method of treating cancer, the method comprising administering to a human in need thereof a therapeutically-effective amount of a compound, wherein the compound binds to a mutant p53 protein and reconforms the mutant p53 protein to a conformation of p53 that exhibits anti-cancer activity, wherein if in a controlled study, the therapeutically-effective amount of the compound is administered to a first subject with a cancer that expresses mutant p53, then a plasma concentration in the first subject of a protein that is a biomarker of wild-type p53 activity when measured from about 8 to about 72 hours after administration of the compound is determined to be at least about 2-fold greater than that determined in a second subject who was not administered the compound, as determined by an enzyme-linked immunosorbent assay.

Disclosed herein is a method of treating cancer, the method comprising: (i) withdrawing a first blood sample from a subject with a cancer that expresses mutant p53; (ii) measuring a first plasma concentration of a protein that is a biomarker of wild-type p53 activity in the first blood sample; (iii) after measuring the first plasma concentration of the protein that is the biomarker of wild-type p53 activity in the first blood sample, administering to the subject a therapeutically-effective amount of a compound that binds to a mutant p53 protein and reconforms the mutant p53 protein to a conformation of p53 that exhibits anti-cancer activity; (iv) withdrawing a second blood sample from the subject after administering the compound; and (v) measuring a second plasma concentration of the protein that is a biomarker of wild-type p53 activity in the second blood sample.

Disclosed herein is a method of treating a cancer, the method comprising administering to a subject in need thereof a therapeutically-effective amount of a compound that binds to a mutant p53 protein in the subject and reconforms the mutant p53 protein to a conformation of p53 that exhibits anti-cancer activity within about 2 hours of contacting the cancer with the compound.

Disclosed herein is a method of treating a cancer, the method comprising administering to a subject in need thereof a therapeutically-effective amount of a compound that binds to a mutant p53 protein and reconforms the mutant p53 protein to a conformation of p53 that exhibits anti-cancer activity, wherein the cancer is heterozygous for a p53 Y220C mutation.

Disclosed herein is a method of treating a cancer, the method comprising administering to a subject in need thereof a therapeutically-effective amount of a compound that binds a mutant p53 protein in the subject, wherein binding of the compound to the mutant p53 protein in the subject modulates at least two genes downstream of p53 in the subject, wherein the genes are APAF1, BAX, BBC3, BIRC5, BRCA2, BRCA1, BTG2, CCNB1, CCNE1, CCNG1, CDC25A, CDC25C, CDK1, CDKN1A, CHEK1, CHEK2, E2F1, EGR1, FAS, GADD45A, GAPDH, GDF15, IL6, MDM2, MSH2, p21, PIDD1, PPM1D, PRC1, SESN2, TNFRSF10B, TNFRSF10D, and TP53.

Disclosed herein is a compound comprising a structure that binds to a mutant p53 protein and increases wild type p53 activity of the mutant p53 protein; wherein if in a controlled study, a therapeutically-effective amount of the compound is administered to a first subject with a cancer that expresses mutant p53, then a plasma concentration in the first subject of a protein that is a biomarker of wild-type p53 activity when measured from about 8 to about 72 hours after administration of the compound is determined to be at least about 2-fold greater than that determined in a second subject who was not administered the compound, as determined by an enzyme-linked immunosorbent assay.

### BRIEF DESCRIPTION OF THE FIGURES

FIG. 1 PANEL A shows IC₅₀ values of the 5-day MTT assay using Compound 2 in human cell lines. PANEL B shows IC₅₀ values of the 5-day MTT assay using Compound 2 in additional mouse cell lines.

FIG. 2 PANEL A and PANEL B show that Compound 2 activates transcription of p53 target genes p21 and MDM2 in a dose-dependent manner in all Y220C mutant p53 carrying cell lines tested, representing diverse tissue origins.

FIG. 3 PANEL A and PANEL B show activity and selectivity of Compound 2 in cells harboring Y220C p53 mutation, but not cells without p53 (KO) or cells with either WT or different mutations of p53.

FIG. 4 PANEL A-PANEL E visualizes transcriptional changes following Compound 2 treatment.

FIG. 5 PANEL A-PANEL D show selectivity of Compound 2 by the 84 p53-related gene panel.

FIG. 6 PANEL A and PANEL B demonstrate elevated p53 (DO-1) levels within a panel of tumor cell lines harboring p53 Y220C compared to levels found within normal and tumor lines containing WT p53, both untreated and treated with 50 nM RG7388 for 24 hours.

FIG. 7 PANEL A depicts a p53 Western blot following an immunoprecipitation using mutant specific or wild type (WT) specific antibodies from lysates treated with varying concentrations of Compound 2 for 2 hours. PANEL B shows analysis of the same lysate using ELISAs developed to quantitate mutant or wild type conformation.

FIG. 8 PANEL A and PANEL B show that addition of cycloheximide (+CHX) did not affect the ability of Compound 2 to induce mutant to wild type conformation change.

FIG. 9 PANEL A and PANEL B show the conversion from mutant p53 to WT conformation p53 after a 4-hour treatment with Compound 2 in 11 Y220C mutant p53 cell lines.

FIG. 10 PANEL A shows NUCG3 patterns of WT conformation p53 following treatment with Compound 2. PANEL B shows T3M4 patterns of WTp53 following treatment with Compound 2.

FIG. 11 PANEL A-PANEL C show conversion of mutant p53 to wild-type conformation in mice treated with vehicle, Compound 1 75 mg/kg, or 150 mg/kg BID×1.

FIG. 12 PANEL A-PANEL D show that wild-type p53 conversion results in downstream increases in p53 target transcripts, p21, MDM2, and MIC-1 (Gdf15) in mice treated with vehicle, Compound 1 75 mg/kg, or 150 mg/kg BID×1.

FIG. 13 PANEL A-PANEL J show results from a p53 pathway profiling panel: average fold change over vehicle in Bbc3, Birc5, Ccng1, Cdc25c, Cdn1a, Chek1, Egr1, 116, Sens2, and Zmat3 mRNA in mice treated with vehicle, Compound 1 75 mg/kg, or 150 mg/kg BID×1.

FIG. 14 PANEL A-PANEL D show results from a NF-κB Qiagen panel that demonstrates average fold changes over vehicle in Bcl2a1a, Ccl2, Csf2, and Egr1 mRNA in mice treated with vehicle or Compound 1 150 mg/kg BID×1 over 144 days.

FIG. 15 shows the individual and mean plasma concentration-time profile of Compound 2 in female CD-1 mice following a single PO administration at 100 mg/kg.

FIG. 16 shows the individual and mean brain concentration-time profile of Compound 2 in female CD-1 mice following a single PO administration at 100 mg/kg.

FIG. 17 shows calibration curves obtained for the test material in the sample run.

FIG. 18 shows tumor volume across study (average±SD) for mice treated with vehicle, Compound 2 300 mg/kg 2Q7D5, or 150 mg/kg 2Q7D×4.

FIG. 19 PANEL A-PANEL C shows individual tumor volumes across a study of 10 mice treated with control, Compound 2 300 mg/kg 2Q7D×5, or 150 mg/kg 2Q7D×4.

FIG. 20 shows average percent change in body weight across a study (%, average±SD) of mice treated with vehicle QD×21, Compound 2 300 mg/kg 2Q7D×5, or 150 mg/kg 2Q7D×4.

FIG. 21 PANEL A-PANEL C show conversion of mutant p53 to wild-type p53 conformation in mice treated with vehicle, Compound 2 300 mg/kg 2Q7D×4, or 150 mg/kg 2Q7D×4.

FIG. 22 PANEL A and PANEL B show that conversion of mutant to WT conformation p53 results in downstream increases in p53 target proteins: p21 and MDM2 in mice treated with Compound 2 300 mg/kg 2Q7D×4 or 150 mg/kg 2Q7D×4.

FIG. 23 shows wild-type p53 conversion results in increased expression of MIC-1 in mice treated with vehicle control, Compound 2 300 mg/kg 2Q7D×4, or 150 mg/kg 2Q7D×4.

FIG. 24 shows tumor volume across study (average±SD) in mice treated with vehicle BID×21, Compound 2 100 mg/kg QD×44, or 300 mg/kg Q3D×11.

FIG. 25 PANEL A-PANEL C show individual tumor volumes across the study in mice treated with vehicle, Compound 2 100 mg/kg QD×44, or 300 mg/kg Q3D×11.

FIG. 26 shows average percent change in body weight across study (%, average±SD) in mice treated with vehicle BID×21, Compound 2 100 mg/kg QD×21, or 300 mg/kg Q3D×6.

FIG. 27 PANEL A-PANEL C show conversion of mutant p53 to wild-type p53 conformation in mice treated with vehicle control or Compound 2 300 mg/kg.

FIG. 28 PANEL A and PANEL B show conversion of mutant to WT conformation p53 results in downstream increases in p53 target proteins: p21 and MDM2 in mice treated with vehicle control or Compound 2 300 mg/kg.

FIG. 29 shows wild-type p53 conversion results in increased expression of MIC-1 in mice treated with vehicle control, Compound 2 100 mg/kg, or 300 mg/kg.

FIG. 30 PANEL A-PANEL C show conversion of mutant p53 to wild-type conformation in mice treated with vehicle control. Compound 2 300 mg/kg 2QD×1, 100 mg/kg QD×1, QD×2, QD×4, or QD×6.

FIG. 31 PANEL A and PANEL B shows wild-type p53 conversion results in downstream increase in p53 target proteins: MDM2 and p21 in mice treated with vehicle control, Compound 2 300 mg/kg 2QD×1, 100 mg/kg QD×1, QD×2, QD×3, or QD×4.

FIG. 32 shows wild-type p53 conversion results in expression of plasma MIC-1 in mice treated with vehicle control, Compound 2 300 mg/kg 2QD×1, 100 mg/kg QD×1, QD×2, QD×3, or QD×4.

FIG. 33 PANEL A-PANEL D show changes in p21, MDM2, and BIRC5 (survivin), and GAPDH gene expression relative to GAPDH in mice treated with vehicle control, Compound 2 300 mg/kg BID×1, 100 mg/kg QD×1, QD×2, QD×3, or QD×4.

FIG. 34 shows changes in p53 target gene expression following daily dosing of Compound 2 at 100 mg/kg.

FIG. 35 shows tumor volume across study (average±SD) in mice treated with vehicle QD×21, Compound 2 25 mg/kg QD×21, 50 mg/kg QD×21, or 100 mg/kg QD×21.

FIG. 36 PANEL A-PANEL D shows individual tumor volumes across study in mice treated with vehicle QD×21, Compound 2 25 mg/kg QD×21, 50 mg/kg QD×21, or 100 mg/kg QD×21.

FIG. 37 shows average percent change in body weight across study (%, average±SD) in mice treated with vehicle QD×21, Compound 2 25 mg/kg QD×21, 50 mg/kg QD×21, or 100 mg/kg QD×21.

FIG. 38 PANEL A-PANEL C show conversion of mutant p53 to wild-type p53 conformation in mice treated with vehicle control, Compound 2 25 mg/kg or 50 mg/kg.

FIG. 39 PANEL A and PANEL B show conversion of mutant to WT conformation p53 results in downstream increases in p53 target proteins: p21 and MDM2 in mice treated with vehicle control, Compound 2 25 mg/kg or 50 mg/kg.

FIG. 40 shows wild-type p53 conversion results in increased expression of MIC-1 in mice treated with vehicle control, Compound 2 25 mg/kg, 50 mg/kg, or 100 mg/kg.

FIG. 41 shows tumor volume across 17 days of study (average±SD) in mice treated with vehicle control, Compound 2 25 mg/kg QD×18, 50 mg/kg QD×18, 100 mg/kg QD×18, 150 mg/kg 2Q7D×4, or 300 mg/kg 2Q7D×4.

FIG. 42 PANEL A-PANEL F show individual tumor volumes across study in individual mice treated with vehicle control, 25 mg/kg QD×18, 50 mg/kg QD×18, 100 mg/kg QD×18, 150 mg/kg 2Q7D×4, or 300 mg/kg 2Q7D×4.

FIG. 43 shows average percent change in body weight (%, average±SD) in mice treated with vehicle control, Compound 2 25 mg/kg QD×18, 50 mg/kg QD×18, 100 mg/kg QD×18, 150 mg/kg 2Q7D×4, or 300 mg/kg 2Q7D×4.

FIG. 44 PANEL A-PANEL C show conversion of mutant p53 to wild-type p53 conformation in mice treated with vehicle control, Compound 2 25 mg/kg QD×18, 50 mg/kg QD×18, 100 mg/kg QD×18, 150 mg/kg 2Q7D×4, or 300 mg/kg 2Q7D×4.

FIG. 45 PANEL A and PANEL B show conversion of mutant to WT conformation p53 results in downstream increases in p53 target proteins: p21 and MDM2 in mice treated with vehicle control, Compound 2 25 mg/kg QD×18, 50 mg/kg QD×18, 100 mg/kg QD×18, 150 mg/kg 2Q7D×4, or 300 mg/kg 2Q7D×4.

FIG. 46 PANEL A-PANEL C show conversion of mutant p53 to wild-type conformation in mice treated with vehicle control, Compound 2 50 mg/kg QD×1, QD×2, QD×4, QD×6, 100 mg/kg QD×1, QD×2, QD×4, or QD×6.

FIG. 47 PANEL A and PANEL B show wild-type p53 conversion results in downstream increase in p53 target proteins: MDM2 and p21 in mice treated with vehicle control, Compound 2 50 mg/kg QD×1, QD×2, QD×4, QD×6, 100 mg/kg QD×1, QD×2, QD×4, or QD×6.

FIG. 48 shows wild-type p53 conversion results in expression of plasma MIC-1 in mice treated with vehicle control, Compound 2 50 mg/kg QD×1, QD×2, QD×4, QD×6, 100 mg/kg QD×1, QD×2, QD×4, or QD×6.

FIG. 49 PANEL A-PANEL D show changes in p21, MDM2, and BIRC5 (survivin) gene expression relative to GAPDH following daily dosing of Compound 2 in mice treated with vehicle control, Compound 2 50 mg/kg QD×1, QD×2, QD×4, QD×6, 100 mg/kg QD×1, QD×2, QD×4, or QD×6.

FIG. 50 shows changes in p53 target gene expression following daily dosing of Compound 2 at 100 mg/kg.

FIG. 51 shows pharmacokinetic response to treatment with Compound 2 100 mg/kg QD×1, 300 mg/kg QD×1, or 300 mg/kg BID (8 hr).

FIG. 52 shows plasma concentrations over time for Compound 2 at three dose levels (25 mg/kg QD×1, 50 mg/kg QD×1, 100 mg/kg QD×1).

FIG. 53 shows plasma concentrations over time for Compound 2 at three dose levels (25 mg/kg QD×1, 50 mg/kg QD×1, 100 mg/kg QD×1).

FIG. 54 shows individual and mean plasma concentration-time profiles of Compound 2 following an intravenous administration at 2.5 mg/kg in female Sprague-Dawley rats.

FIG. 55 shows individual and mean plasma concentration-time profiles of Compound 2 following an oral administration at 50 mg/kg in female Sprague-Dawley rats.

FIG. 56 shows individual and mean plasma concentration-time profiles of Compound 2 following an oral administration at 300 mg/kg in female Sprague-Dawley rats.

FIG. 57 shows a comparison of plasma concentration of Compound 2 following IV (2.5 mg/kg) and PO (50 mg/kg or 300 mg/kg) administration in female Sprague-Dawley rats.

FIG. 58 shows mean plasma concentration profiles of Compound 2 in male beagle dogs following single intravenous bolus and oral administrations of Compound 2 at 2.5, 25, and 100 mg/kg in phases 1, 2, and 3.

FIG. 59 shows individual and mean plasma concentration profiles of Compound 2 in male beagle dogs following single intravenous bolus administration of Compound 2 at 2.5 mg/kg in phase 1.

FIG. 60 shows individual and mean plasma concentration profiles of Compound 2 in male beagle dogs following single oral bolus administration of Compound 2 at 25 mg/kg in phase 2.

FIG. 61 shows individual and mean plasma concentration profiles of Compound 2 in male beagle dogs following single oral bolus administration of Compound 2 at 100 mg/kg in phase 3.

FIG. 62 shows mean plasma concentration profiles of Compound 2 in male Cynomologous monkeys following single intravenous bolus and oral administrations of Compound 2 at 2.5, 25, and 100 mg/kg in phases 1, 2, and 3.

FIG. 63 shows individual and mean plasma concentration profiles of Compound 2 in male Cynomolgus monkeys following single intravenous bolus administration of Compound 2 at 2.5 mg/kg in phase 1.

FIG. 64 shows individual and mean plasma concentration profiles of Compound 2 in male Cynomolgus monkeys following single oral bolus administration of Compound 2 at 25 mg/kg in phase 2.

FIG. 65 shows individual and mean plasma concentration profiles of Compound 2 in male Cynomolgus monkeys following single oral bolus administration of Compound 2 at 100 mg/kg in phase 3.

FIG. 66 illustrates CYP activity vs. Compound 2 concentration curves.

FIG. 67 illustrates CYP activity vs positive inhibitor concentration curves.

FIG. 68 illustrates CYP activity vs Compound 2 concentration curves.

FIG. 69 illustrates CYP activity vs Positive TDI Concentration curves.

FIG. 70 PANEL A and PANEL B illustrate Day 1 and 10 PK Profile of Compound 2 and Metabolites (Compounds 3-8) 0-24 h in Rats Following 50 mg/kg QD×10 Dose.

### DETAILED DESCRIPTION

The present invention provides compounds and methods for restoring wild-type function to mutant p53. The compounds of the present invention can bind to mutant p53 and restore the ability of the p53 mutant to bind DNA. The restoration of activity of the p53 mutant can allow for the activation of downstream effectors of p53 leading to inhibition of cancer progression. The invention further provides methods of treatment of a cancerous lesion or a tumor harboring a p53 mutation.

Cancer is a collection of related diseases characterized by uncontrolled proliferation of cells with the potential to metastasize throughout the body. Cancer can be classified into five broad categories including, for example: carcinomas, which can arise from cells that cover internal and external parts of the body such as the lung, breast, and colon; sarcomas, which can arise from cells that are located in bone, cartilage, fat, connective tissue, muscle, and other supportive tissues; lymphomas, which can arise in the lymph nodes and immune system tissues; leukemia, which can arise in the bone marrow and accumulate in the bloodstream; and adenomas, which can arise in the thyroid, the pituitary gland, the adrenal gland, and other glandular tissues.

Although different cancers can develop in virtually any of the body's tissues, and contain unique features, the basic processes that cause cancer can be similar in all forms of the disease. Cancer begins when a cell breaks free from the normal restraints on cell division and begins to grow and divide out of control. Genetic mutations in the cell can preclude the ability of the cell to repair damaged DNA or initiate apoptosis, and can result in uncontrolled growth and division of cells.

The ability of tumor cell populations to multiply is determined not only by the rate of cell proliferation but also by the rate of cell attrition. Programmed cell death, or apoptosis, represents a major mechanism of cellular attrition. Cancer cells can evade apoptosis through a variety of strategies, for example, through the suppression of p53 function, thereby suppressing expression of pro-apoptotic proteins.

Oncogenes and tumor suppressor genes can regulate the proliferation of cells. Genetic mutations can affect oncogenes and tumor suppressors, potentially activating or suppressing activity abnormally, further facilitating uncontrolled cell division. Whereas oncogenes assist in cellular growth, tumor suppressor genes slow cell division by repairing damaged DNA and activating apoptosis. Cellular oncogenes that can be mutated in cancer include, for example, Cdk1, Cdk2, Cdk3, Cdk4, Cdk6, EGFR, PDGFR, VEGF, HER2, Raf kinase, K-Ras, and myc. Tumor suppressor genes that can be mutated in cancer include, for example, BRCA1, BRCA2, cyclin-dependent kinase inhibitor 1C, Retinoblastoma protein (pRb), PTEN, p16, p27, p53, and p73.

### Tumor Suppressor p53.

The tumor suppressor protein p53 is a 393 amino acid transcription factor that can regulate cell growth in response to cellular stresses including, for example, UV radiation, hypoxia, oncogene activation, and DNA damage. p53 has various mechanisms for inhibiting the progression of cancer including, for example, initiation of apoptosis, maintenance of genomic stability, cell cycle arrest, induction of senescence, and inhibition of angiogenesis. Due to the critical role of p53 in tumor suppression, p53 is inactivated in almost all cancers either by direct mutation or through perturbation of associated signaling pathways involved in tumor suppression. Homozygous loss of the p53 gene occurs in almost all types of cancer, including carcinomas of the breast, colon, and lung. The presence of certain p53 mutations in several types of human cancer can correlate with less favorable patient prognosis.

In the absence of stress signals, p53 levels are maintained at low levels via the interaction of p53 with Mdm2, an E3 ubiquitin ligase. In an unstressed cell, Mdm2 can target p53 for degradation by the proteasome. Under stress conditions, the interaction between Mdm2 and p53 is disrupted, and p53 accumulates. The critical event leading to the activation of p53 is phosphorylation of the N-terminal domain of p53 by protein kinases, thereby transducing upstream stress signals. The phosphorylation of p53 leads to a conformational change, which can promote DNA binding by p53 and allow transcription of downstream effectors. The activation of p53 can induce, for example, the intrinsic apoptotic pathway, the extrinsic apoptotic pathway, cell cycle arrest, senescence, and DNA repair. p53 can activate proteins involved in the above pathways including, for example, Fas/Apo1, KILLER/DR5, Bax, Puma, Noxa, Bid, caspase-3, caspase-6, caspase-7, caspase-8, caspase-9, and p21 (WAF1). Additionally, p53 can repress the transcription of a variety of genes including, for example, c-MYC, Cyclin B, VEGF, RAD51, and hTERT.

Each chain of the p53 tetramer is composed of several functional domains including the transactivation domain (amino acids 1-100), the DNA-binding domain (amino acids 101-306), and the tetramerization domain (amino acids 307-355), which are highly mobile and largely unstructured. Most p53 cancer mutations are located in the DNA-binding core domain of the protein, which contains a central β-sandwich of anti-parallel β-sheets that serves as a basic scaffold for the DNA-binding surface. The DNA-binding surface is composed of two β-turn loops, L2 and L3, which are stabilized by a zinc ion, for example, at Arg175 and Arg248, and a loop-sheet-helix motif. Altogether, these structural elements form an extended DNA-binding surface that is rich in positively-charged amino acids, and makes specific contact with various p53 response elements.

Due to the prevalence of p53 mutations in virtually every type of cancer, the reactivation of wild type p53 function in a cancerous cell can be an effective therapy. Mutations in p53 located in the DNA-binding domain of the protein or periphery of the DNA-binding surface result in aberrant protein folding required for DNA recognition and binding. Mutations in p53 can occur, for example, at amino acids Val143, His168, Arg175, Tyr220, Gly245, Arg248, Arg249, Phe270, Arg273, and Arg282. p53 mutations that can abrogate the activity of p53 include, for example, R175H, Y220C, G245S, R248Q, R248W, R273H, and R282H. These p53 mutations can either distort the structure of the DNA-binding site or thermodynamically destabilize the folded protein at body temperature. Wild-type function of p53 mutants can be recovered by binding of the p53 mutant to a compound that can shift the folding-unfolding equilibrium towards the folded state, thereby reducing the rate of unfolding and destabilization.

Non-limiting examples of amino acids include: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, lie); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val).

### Mechanism of Compounds of the Invention.

The compounds of the present invention can selectively bind to a p53 mutant and can recover wild-type activity of the p53 mutant including, for example, DNA binding function and activation of downstream targets involved in tumor suppression. In some embodiments, a compound of the invention selectively binds to the p53 Y220C mutant. The Y220C mutant is a temperature sensitive mutant, which binds to DNA at lower temperature and is denatured at body temperature. A compound of the invention can stabilize the Y220C mutant to reduce the likelihood of denaturation of the protein at body temperature.

In some embodiments, the compounds of the disclosure stabilize a mutant p53 and allows the mutant p53 to bind to DNA, thereby shifting the equilibrium of wild type and mutant p53 proteins to wild type p53. In some embodiments, the compounds of the disclosure reactivate the mutant p53 protein to provide wild type p53 activity. In some embodiments, the compounds of the disclosure reactivate the mutant p53 protein to provide pro-apoptotic p53 activity. In some embodiments, the compounds of the disclosure reactivate the mutant p53 protein to block angiogenesis. In some embodiments, the compounds of the disclosure reactivate the mutant p53 protein to induce cellular senescence. In some embodiments, the compounds of the disclosure reactivate the mutant p53 protein to induce cell cycle arrest.

The compounds of the disclosure can reconform mutant p53 to a conformation of p53 that exhibits anti-cancer activity. In some embodiments, the mutant p53 is reconformed to a wild type conformation p53. In some embodiments, the mutant p53 is reconformed to a pro-apoptotic conformation of p53. In some embodiments, the mutant p53 is reconformed to a conformation of p53 that blocks angiogenesis. In some embodiments, the mutant p53 is reconformed to a conformation of p53 that induces cellular senescence. In some embodiments, the mutant p53 is reconformed to a conformation of p53 that induces cell-cycle arrest.

Located in the periphery of the p53 β-sandwich connecting β-strands S7 and S8, the aromatic ring of Y220 is an integral part of the hydrophobic core of the β-sandwich. The Y220C mutation can be highly destabilizing, due to the formation of an internal surface cavity. A compound of the invention can bind to and occupy this surface crevice to stabilize the β-sandwich, thereby restoring wild-type p53 DNA-binding activity.

To determine the ability of a compound of the invention to bind and stabilize mutant p53, assays can be employed to detect, for example, a conformational change in the p53 mutant or activation of wild-type p53 targets. Conformational changes in p53 can be measured by, for example, differential scanning fluorimetry (DSF), isothermal titration calorimetry (ITC), nuclear magnetic resonance spectrometry (NMR), or X-ray crystallography. Additionally, antibodies specific for the wild type of mutant conformation of p53 can be used to detect a conformational change via, for example, immunoprecipitation (IP), immunofluorescence (IF), or immunoblotting.

Methods used to detect the ability of the p53 mutant to bind DNA can include, for example, DNA affinity immunoblotting, modified enzyme-linked immunosorbent assay (ELISA), electrophoretic mobility shift assay (EMSA), fluorescence resonance energy transfer (FRET), homogeneous time-resolved fluorescence (HTRF), and a chromatin immunoprecipitation (ChIP) assay.

To determine whether a compound described herein is able to reactivate the transcriptional activity of p53, the activation of downstream targets in the p53 signaling cascade can be measured. Activation of p53 effector proteins can be detected by, for example, immunohistochemistry (IHC-P), reverse transcription polymerase chain reaction (RT-PCR), and western blotting. The activation of p53 can also be measured by the induction of apoptosis via the caspase cascade and using methods including, for example, Annexin V staining, TUNEL assays, pro-caspase and caspase levels, and cytochrome c levels. Another consequence of p53 activation is senescence, which can be measured using methods such as β-galactosidase staining.

A p53 mutant that can be used to determine the effectiveness of a compound of the invention to increase the DNA binding ability of a p53 mutant is a p53 truncation mutant, which contains only amino acids 94-312, encompassing the DNA-binding domain of p53. For example, the sequence of the p53 Y220C mutant used for testing compound efficacy can be:

(SEQ ID NO. 1)
SSSVPSQ
KTYQGSYGFR LGFLHSGTAK SVTCTYSPAL NKMFCQLAKT
CPVQLWVDST PPPGTRVRAM AIYKQSQHMT EVVRRCPHHE
RCSDSDGLAP PQHLIRVEGN LRVEYLDDRN TFRHSVVVPC
EPPEVGSDCT TIHYNYMCNS SCMGGMNRRP ILTIITLEDS
SGNLLGRNSF EVHVCACPGR DRRTEEENLR KKGEPHHELP
PGSTKRALSN NT

A compound of the invention can increase the ability of a p53 mutant to bind DNA by at least or up to about 0.1%, at least or up to about 0.2%, at least or up to about 0.3%, at least or up to about 0.4%, at least or up to about 0.5%, at least or up to about 0.6%, at least or up to about 0.7%, at least or up to about 0.8%, at least or up to about 0.9%, at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 21%, at least or up to about 22%, at least or up to about 23%, at least or up to about 24%, at least or up to about 25%, at least or up to about 26%, at least or up to about 27%, at least or up to about 28%, at least or up to about 29%, at least or up to about 30%, at least or up to about 31%, at least or up to about 32%, at least or up to about 33%, at least or up to about 34%, at least or up to about 35%, at least or up to about 36%, at least or up to about 37%, at least or up to about 38%, at least or up to about 39%, at least or up to about 40%, at least or up to about 41%, at least or up to about 42%, at least or up to about 43%, at least or up to about 44%, at least or up to about 45%, at least or up to about 46%, at least or up to about 47%, at least or up to about 48%, at least or up to about 49%, at least or up to about 50%, at least or up to about 51%, at least or up to about 52%, at least or up to about 53%, at least or up to about 54%, at least or up to about 55%, at least or up to about 56%, at least or up to about 57%, at least or up to about 58%, at least or up to about 59%, at least or up to about 60%, at least or up to about 61%, at least or up to about 62%, at least or up to about 63%, at least or up to about 64%, at least or up to about 65%, at least or up to about 66%, at least or up to about 67%, at least or up to about 68%, at least or up to about 69%, at least or up to about 70%, at least or up to about 71%, at least or up to about 72%, at least or up to about 73%, at least or up to about 74%, at least or up to about 75%, at least or up to about 76%, at least or up to about 77%, at least or up to about 78%, at least or up to about 79%, at least or up to about 80%, at least or up to about 81%, at least or up to about 82%, at least or up to about 83%, at least or up to about 84%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, at least or up to about 100%, at least or up to about 125%, at least or up to about 150%, at least or up to about 175%, at least or up to about 200%, at least or up to about 225%, or at least or up to about 250% as compared to the ability of the p53 mutant to bind DNA in the absence of a compound of the invention.

A compound described herein can increase the activity of the p53 mutant that is, for example, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 16-fold, at least or up to about 17-fold, at least or up to about 18-fold, at least or up to about 19-fold, at least or up to about 20-fold, at least or up to about 25-fold, at least or up to about 30-fold, at least or up to about 35-fold, at least or up to about 40-fold, at least or up to about 45-fold, at least or up to about 50-fold, at least or up to about 55-fold, at least or up to about 60-fold, at least or up to about 65-fold, at least or up to about 70-fold, at least or up to about 75-fold, at least or up to about 80-fold, at least or up to about 85-fold, at least or up to about 90-fold, at least or up to about 95-fold, at least or up to about 100-fold, at least or up to about 110-fold, at least or up to about 120-fold, at least or up to about 130-fold, at least or up to about 140-fold, at least or up to about 150-fold, at least or up to about 160-fold, at least or up to about 170-fold, at least or up to about 180-fold, at least or up to about 190-fold, at least or up to about 200-fold, at least or up to about 250-fold, at least or up to about 300-fold, at least or up to about 350-fold, at least or up to about 400-fold, at least or up to about 450-fold, at least or up to about 500-fold, at least or up to about 550-fold, at least or up to about 600-fold, at least or up to about 650-fold, at least or up to about 700-fold, at least or up to about 750-fold, at least or up to about 800-fold, at least or up to about 850-fold, at least or up to about 900-fold, at least or up to about 950-fold, at least or up to about 1,000-fold, at least or up to about 1,500-fold, at least or up to about 2.000-fold, at least or up to about 3,000-fold, at least or up to about 4,000-fold, at least or up to about 5.000-fold, at least or up to about 6,000-fold, at least or up to about 7,000-fold, at least or up to about 8.000-fold, at least or up to about 9,000-fold, or at least or up to about 10,000-fold greater than the activity of the p53 mutant in the absence of the compound.

A compound of the invention can be used, for example, to induce apoptosis, cell cycle arrest, or senescence in a cell. In some embodiments, the cell is a cancer cell. In some embodiments, the cell carries a mutation in p53.

### Compounds of the Invention.

In some embodiments, a compound of the disclosure comprises a substituted heterocyclyl group, wherein the compound binds a mutant p53 protein and increases wild-type p53 activity of the mutant protein. In some embodiments, a compound of the disclosure comprises a heterocyclyl group comprising a halo substituent, wherein the compound binds a mutant p53 protein and increases wild-type p53 activity of the mutant protein. In some embodiments, the compound further comprises an indole group. In some embodiments, the indole group has a 1,1,1,-trifluoroethyl substituent at a 1-position of the indole group.

In some embodiments, the indole group has a propargyl substituent at a 2-position of the indole group. In some embodiments, the propargyl substituent is attached to the indole group via an sp carbon atom of the propargyl substituent. In some embodiments, the propargyl substituent is attached to a nitrogen atom of an aniline group via a methylene group of the propargyl substituent. In some embodiments, the indole group comprises an amino substituent at a 4-position of the indole group. In some embodiments, the amino substituent is attached to the heterocyclyl group. In some embodiments, the heterocyclyl group is a piperidine group. In some embodiments, the halo substituent is a fluoro group. In some embodiments, the halo substituent is a chloro group. In some embodiments, the compound has oral bioavailability that is at least about 50% greater than that of an analogous compound that lacks the halo substituent on the heterocyclyl group.

Non-limiting examples of compounds of the invention include compounds of any of the following formulae:

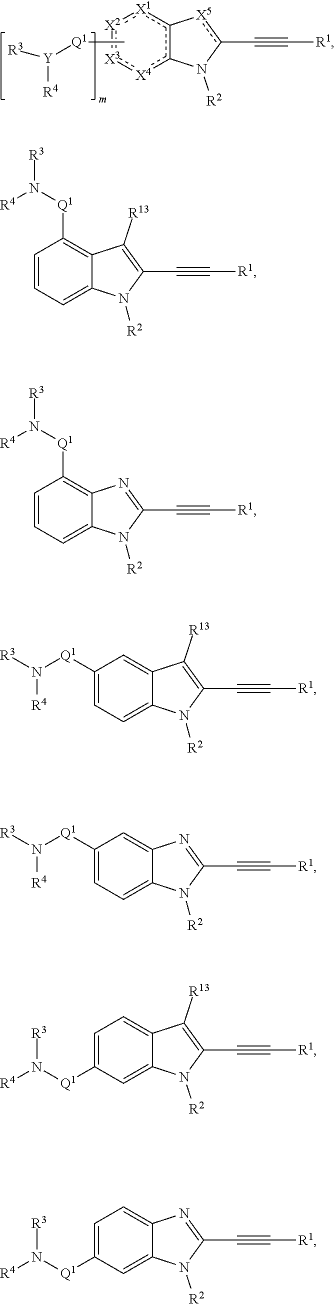

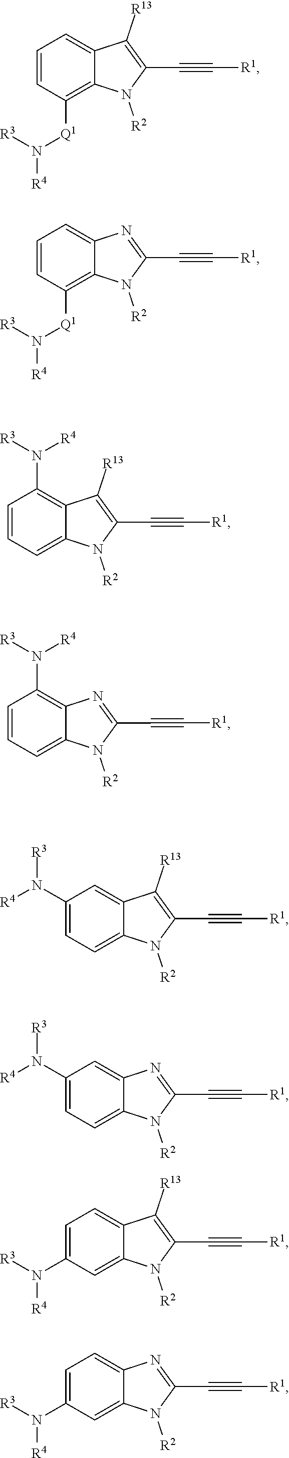

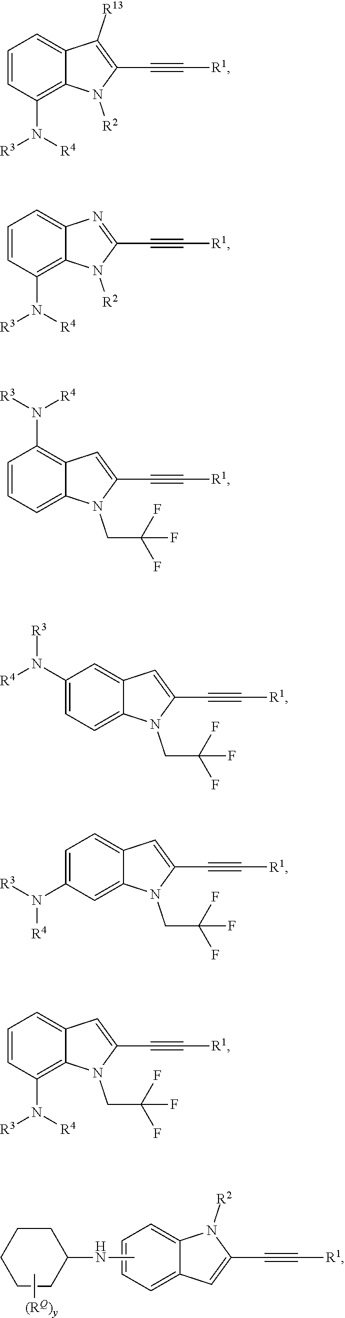

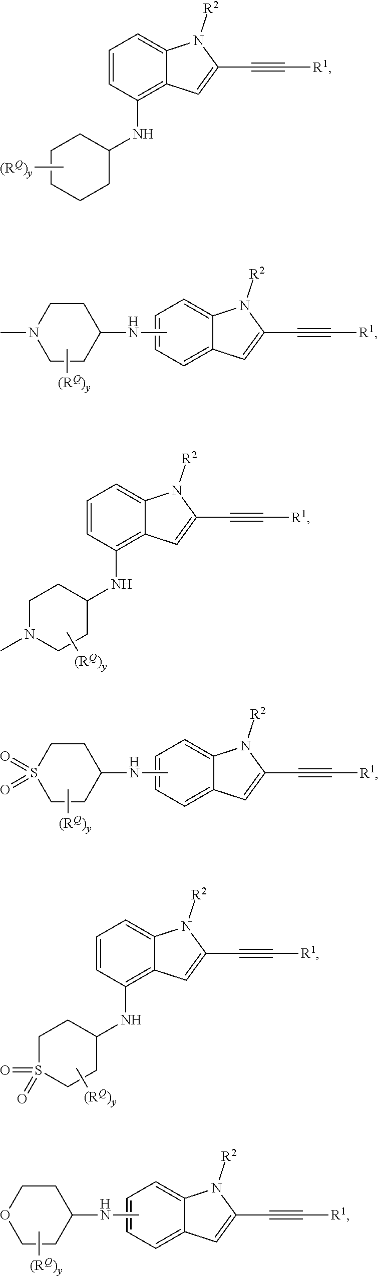

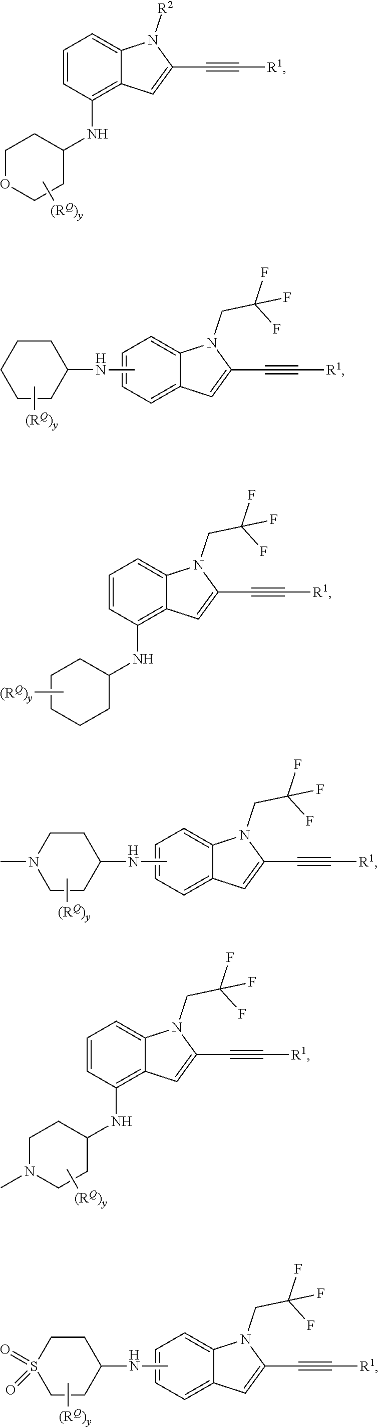

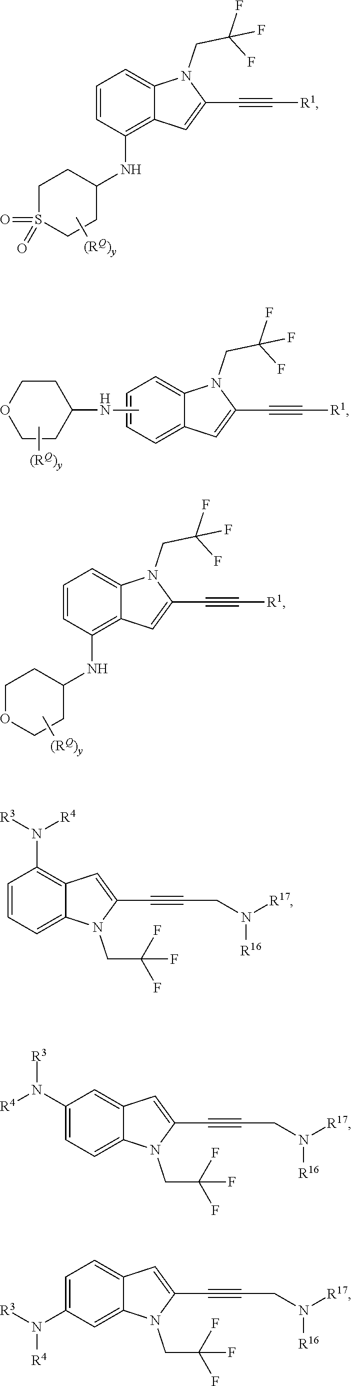

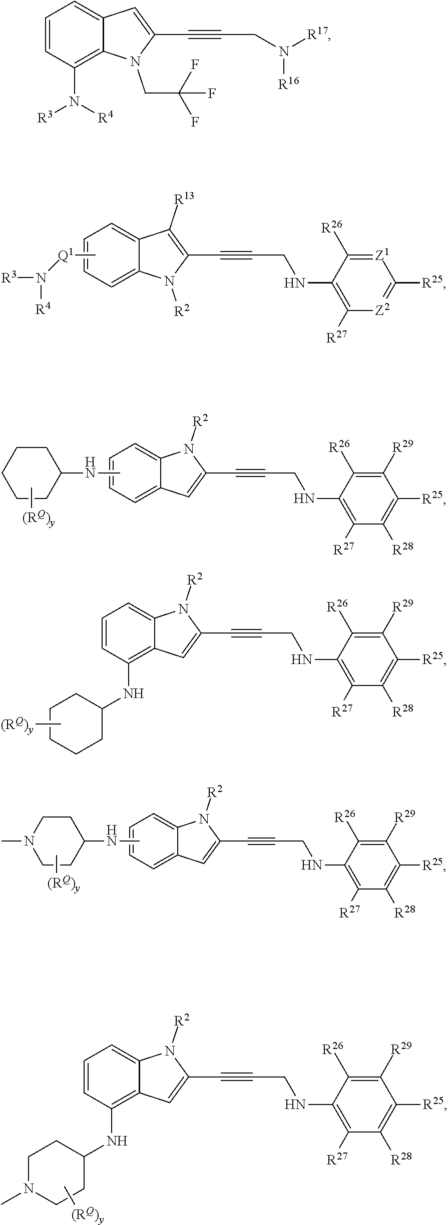

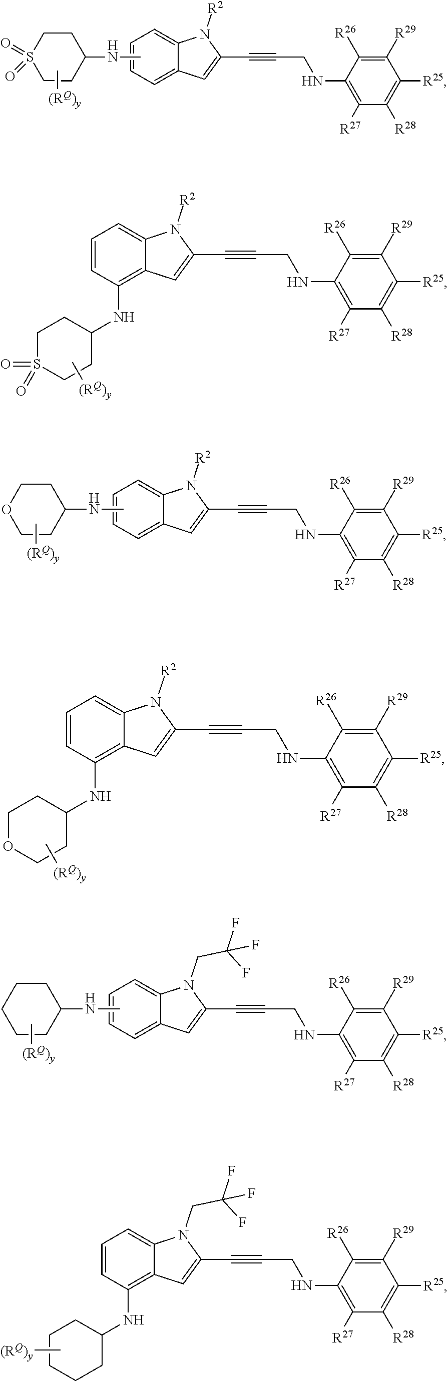

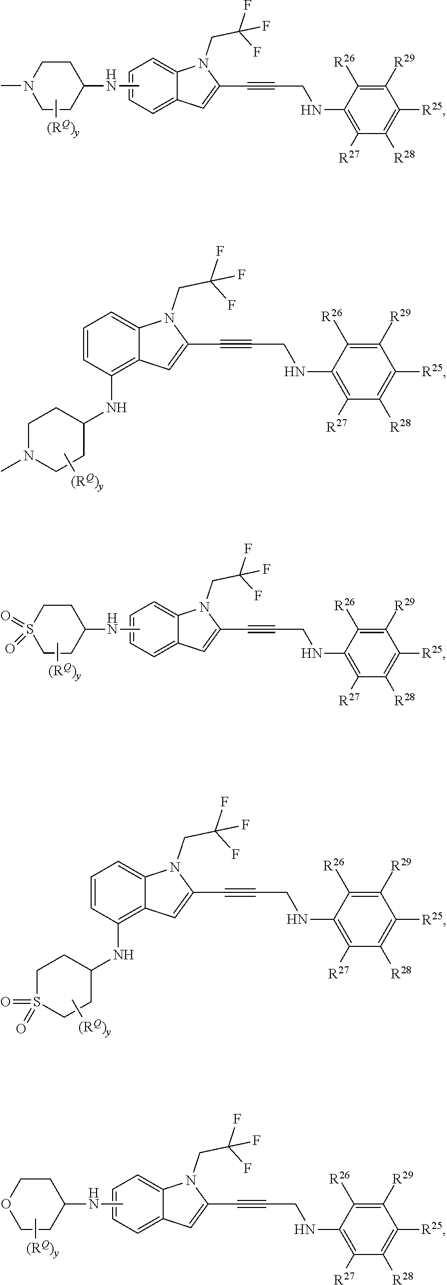

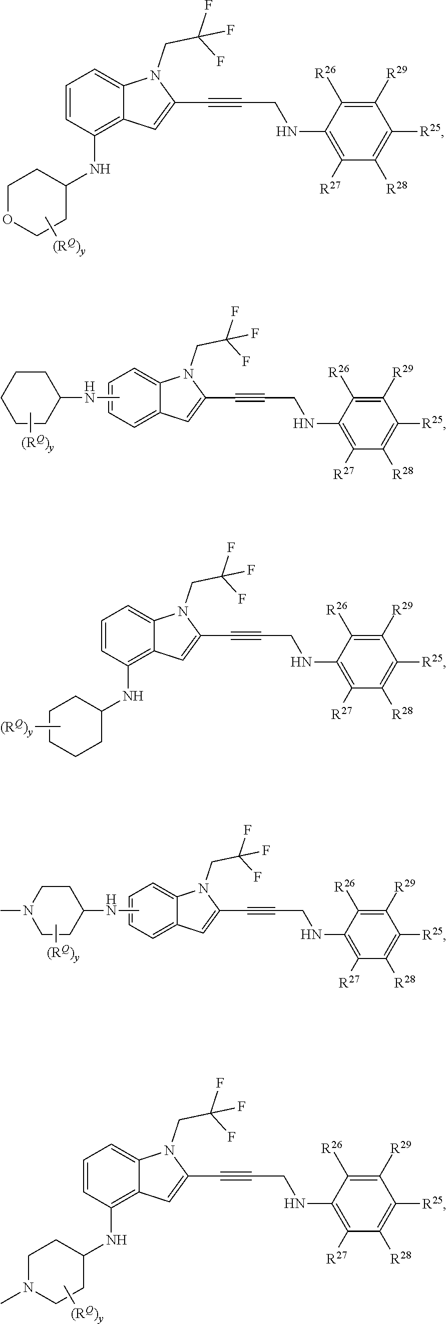

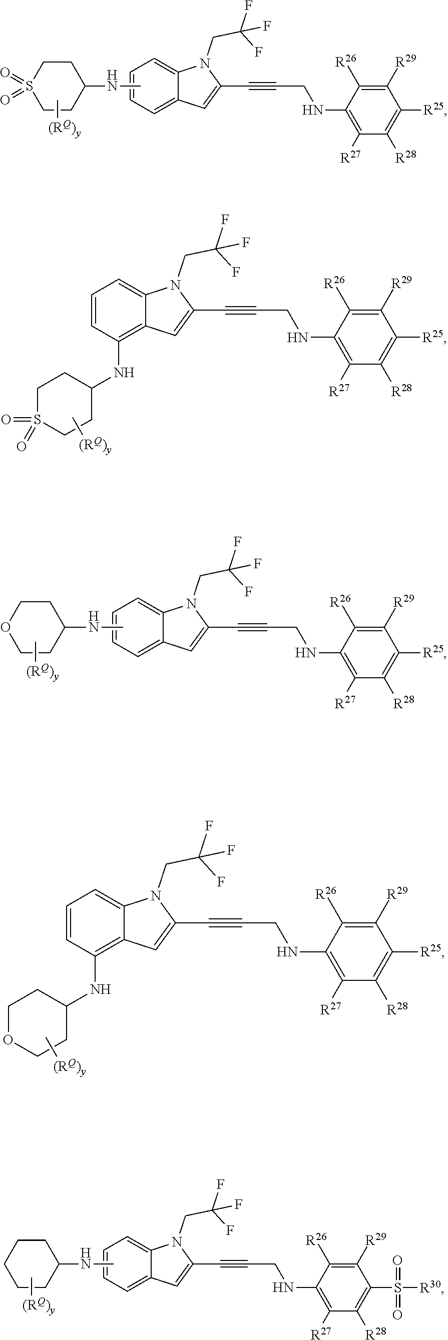

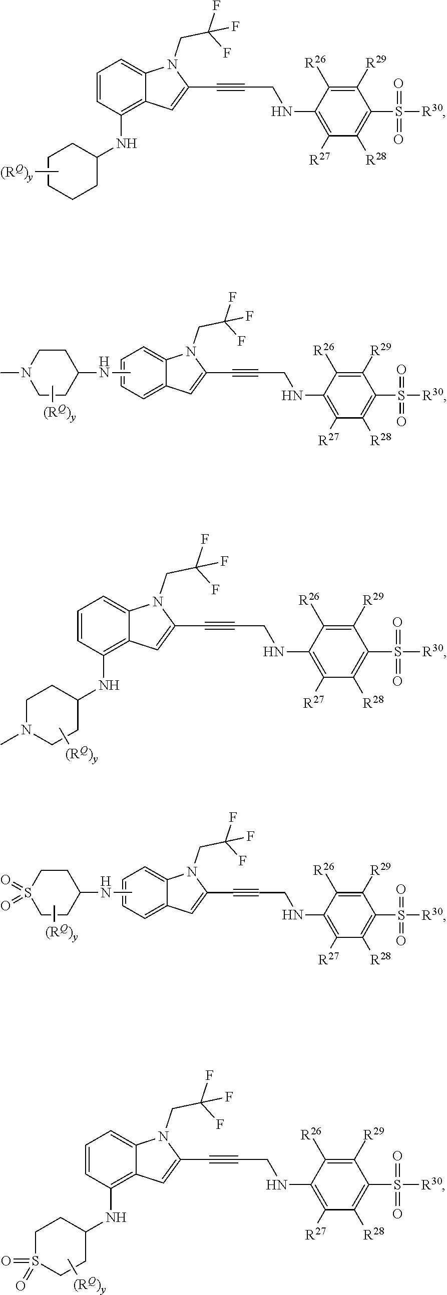

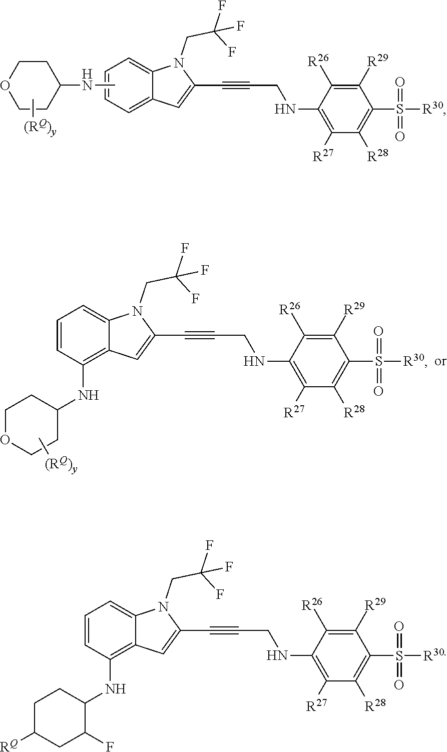

In some embodiments, the compound is of the formula:

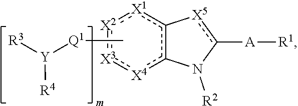

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, A is alkylene, alkenylene, or alkynylene, each of which is substituted or unsubstituted. In some embodiments, A is alkylene. In some embodiments, A is alkenylene. In some embodiments, A is alkynylene.

In some embodiments, A is aryl, heteroaryl, or heterocyclyl, each of which is substituted or unsubstituted. In some embodiments, A is substituted aryl. In some embodiments, A is substituted heteroaryl. In some embodiments, A is substituted heterocyclyl.

In some embodiments, R¹ is alkyl, alkenyl, —C(O)R¹⁶, —C(O)OR¹⁶, or —C(O)NR¹⁶R¹⁷, each of which is unsubstituted or substituted. In some embodiments, R¹ is substituted alkyl. In some embodiments, R¹ is alkyl substituted with NR¹⁶R¹⁷.

In some embodiments, the compound of the formula is:

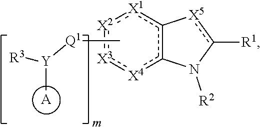

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
ring A is a cyclic group;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, or hydrogen;
R³ is —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted, or hydrogen, or R³ and A together with the nitrogen atom to which R³ and A are bound form a ring, wherein the ring is substituted or unsubstituted, or R³ is absent,
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, a compound of the invention is a compound of the formula

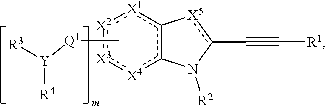

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, or hydrogen;
each R³ and R⁴ is independently, —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent,
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

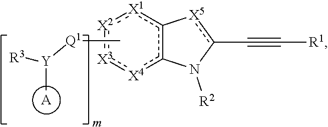

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
ring A is a cyclic group;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, or hydrogen;
R³ is —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted, or hydrogen, or R³ and A together with the nitrogen atom to which R³ and A are bound form a ring, wherein the ring is substituted or unsubstituted, or R³ is absent,
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the pattern of dashed bonds is chosen to provide an aromatic system, for example, an indole, an indolene, a pyrrolopyridine, a pyrrolopyrimidine, or a pyrrolopyrazine.

In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Q¹ is a bond. In some embodiments, Q¹ is C₁-alkylene. In some embodiments, m is 1. In some embodiments, m is 2. In some embodiments, m is 3. In some embodiments, m is 4.

In some embodiments, R¹ is alkyl, alkenyl, —C(O)R¹⁶, —C(O)OR¹⁶, or —C(O)NR¹⁶R¹⁷, each of which is unsubstituted or substituted. In some embodiments, R¹ is substituted alkyl. In some embodiments, R¹ is alkyl substituted with NR¹⁶R¹⁷.

In some embodiments, ring A is aryl, heteroaryl, or heterocyclyl, each of which is substituted or unsubstituted. In some embodiments, ring A is substituted aryl. In some embodiments, ring A is aryl substituted with fluoro-. In some embodiments, ring A is aryl substituted with chloro-. In some embodiments, ring A is substituted heteroaryl, In some embodiments, ring A is heteroaryl substituted with fluoro-. In some embodiments, ring A is heteroaryl substituted with chloro-. In some embodiments, ring A is substituted heterocyclyl. In some embodiments, ring A is heterocyclyl substituted with fluoro-. In some embodiments, A is heterocyclyl substituted with chloro-.

In some embodiments, ring A is piperidinyl, piperazinyl, tetahydropyranyl, morpholinyl, or pyrrolidinyl, each of which is independently substituted or unsubstituted. In some embodiments, ring A is piperidinyl, piperazinyl, tetahydropyranyl, morpholinyl, or pyrrolidinyl, each of which is independently substituted with at least halo-. In some embodiments, ring A is piperidinyl substituted with halo-. In some embodiments, ring A is methylpiperidinyl substituted with halo-. In some embodiments, ring A is 3-fluoro-1-methylpiperidinyl. In some embodiments, ring A is 3-fluoro-1-(2-hydroxy-3-methoxypropyl)piperidinyl. In some embodiments, ring A is tetrahydropyranyl substituted with at least halo-.

In some embodiments, each R¹⁶ and R¹⁷ is independently alkyl, alkenyl, aryl, heteroaryl, heterocyclyl, each of which is independently substituted or unsubstituted, or hydrogen. In some embodiments, R¹⁶ is hydrogen or alkyl. In some embodiments, R¹⁷ is aryl, heteroaryl, or heterocyclyl, each of which is independently substituted or unsubstituted. In some embodiments, R¹⁷ is substituted aryl. In some embodiments, R¹⁷ is substituted phenyl. In some embodiments, R¹⁷ is phenyl substituted with a sulfoxide group, carboxyl group, amide group, amino group, alkyl, alkoxy, hydroxy, halo, cyano, or heterocyclyl, each of which is independently substituted or unsubstituted. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is phenyl substituted with a substituted amide group.

In some embodiments, the compound is of the formula:

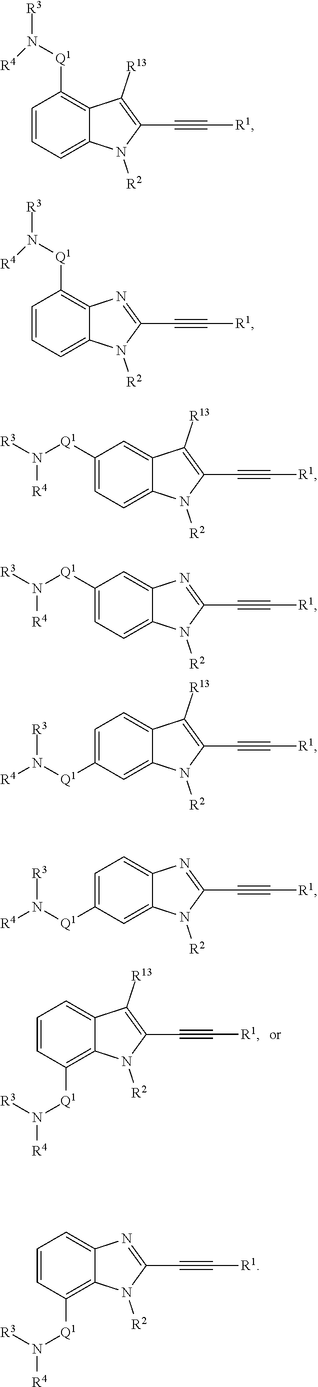

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene or a bond. In some embodiments, Q¹ is C₁-alkylene. In some embodiments, Q¹ is a bond.

In some embodiments, Y is N. In some embodiments, Y is O. In some embodiments, Y is absent.

In some embodiments, R² is hydrogen or alkyl. In some embodiments, R² is alkyl. In some embodiments, R² is substituted C₁-C₅-alkyl. In some embodiments, R² is trifluoroethyl. In some embodiments, R² is cycloalkyl. In some embodiments, R² is cyclopropyl.

In some embodiments, R¹³ is alkyl, alkenyl, hydrogen, or halogen. In some embodiments, R¹³ is hydrogen.

In some embodiments, R² is C₁-C₅-alkyl, and R¹³ is C₁-C₅-alkyl. In some embodiments, R² is C₁-C₅-alkyl, and R¹³ is hydrogen. In some embodiments, R² is substituted C₁-C₅-alkylene. In some embodiments, R² is methyl, ethyl, propyl, iso-propyl, butyl, or tert-butyl, each of which is substituted or unsubstituted. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, the compound is of the formula:

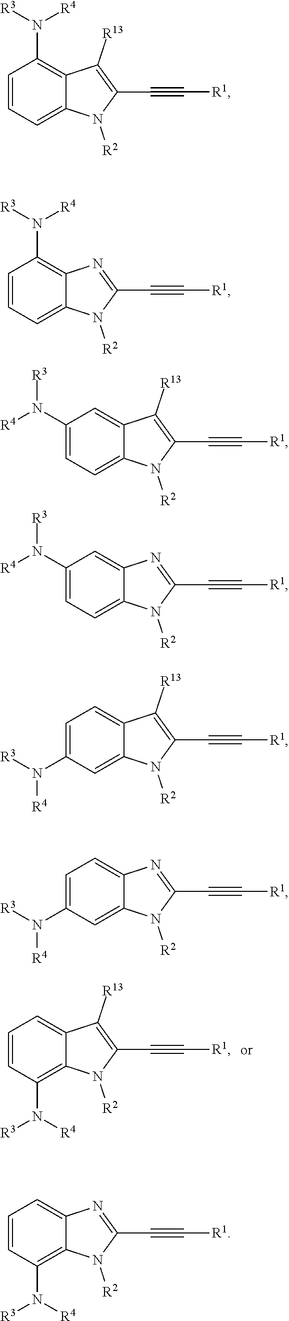

In some embodiments, the compound is of the formula:

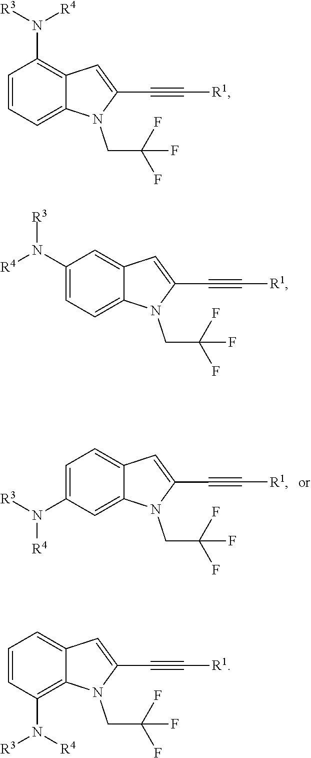

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted. In some embodiments, each R³ and R⁴ is independently substituted or unsubstituted C₁-C₆-alkylene. In some embodiments, R³ is H, and R⁴ is substituted or unsubstituted C₁-C₄ alkylene. In some embodiments, R³ is H, and R⁴ is substituted or unsubstituted heterocyclyl. In some embodiments, R³ is H, and R⁴ is substituted or unsubstituted piperidinyl. In some embodiments, R³ is H, and R⁴ is substituted or unsubstituted cycloalkyl. In some embodiments, R³ is H, and R⁴ is cycloalkyl substituted with an amino group. In some embodiments, R³ is H, and R⁴ is substituted or unsubstituted cyclobutyl. In some embodiments, R³ is H, and R⁴ is cyclobutyl substituted with an amino group. In some embodiments, R³ is H, and R⁴ is substituted or unsubstituted cyclohexyl. In some embodiments, R³ is H, and R⁴ is cyclohexyl substituted with an amino group.

In some embodiments, the compound is of the formula:

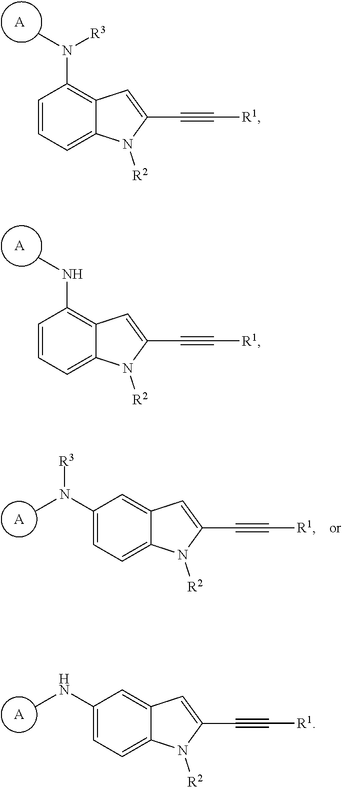

In some embodiments, the compound is of the formula:

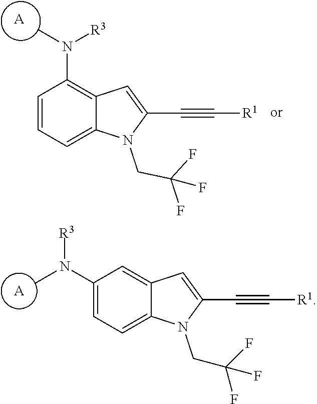

R¹ can be a group substituted with one or more substituents selected from a hydroxyl group, sulfhydryl group, halogens, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group. In some embodiments, R¹ is alkyl, alkenyl, —C(O)R¹⁶, —C(O)OR¹⁶, or —C(O)NR¹⁶R¹⁷.

In some embodiments, R¹ is substituted or unsubstituted C₁-C₃ alkyl. In some embodiments, R¹ is C₁-C₃-alkyl substituted with an amine group. In some embodiments, R¹ is C₁-alkyl substituted with NR¹⁶R¹⁷. In some embodiments, each R¹⁶ and R¹⁷ is independently aryl, heteroaryl, or heterocyclyl, each of which is independently substituted or unsubstituted, or hydrogen. In some embodiments, R¹⁶ is H, and R¹⁷ is substituted aryl. In some embodiments, R¹⁶ is H, and R¹⁷ is substituted phenyl. In some embodiments, R¹⁶ is H, and R¹⁷ is phenyl substituted with alkyl, alkoxy, halo, sulfonamide, a sulfone, or a carboxy group. In some embodiments, R¹⁶ is H, and R¹⁷ is substituted heteroaryl. In some embodiments, R¹⁶ is H, and R¹⁷ is substituted heterocyclyl.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is C₁-alkylene, R¹⁶ is aryl, and R¹⁷ is alkyl. In some embodiments, Q¹ is C₁-alkylene, R¹⁶ is aryl, and R¹⁷ is hydrogen. In some embodiments, Q¹ is C₁-alkylene, R¹⁶ is heteroaryl, and R¹⁷ is alkyl. In some embodiments, Q¹ is C₁-alkylene, R¹⁶ is heteroaryl, and R¹⁷ is hydrogen. In some embodiments, Q¹ is C₁-alkylene, R¹⁶ is substituted heteroaryl, and R¹⁷ is hydrogen. In some embodiments, Q¹ is C₁-alkylene, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano.

In some embodiments, R² is alkyl, and R¹³ is alkyl, each of which is substituted or substituted. In some embodiments, R² is hydrogen, and R¹³ is unsubstituted or substituted alkyl. In some embodiments, R² is methyl, ethyl, propyl, iso-propyl, butyl, or tert-butyl, each of which is substituted or unsubstituted. In some embodiments, R¹³ is methyl, ethyl, propyl, iso-propyl, butyl or tert-butyl. In some embodiments, R² is hydrogen, and R¹³ is hydrogen. In some embodiments, R² is hydrogen, and R¹³ is alkyl. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen. In some embodiments, R³ is alkyl, alkenyl, alkynyl, aryl, heteroaryl, or heterocyclyl, each of which is independently substituted or unsubstituted, or hydrogen. In some embodiments, R³ is substituted alkyl. In some embodiments, R³ is H.

In some embodiments, R³ is H, and R⁴ is unsubstituted or substituted alkyl. In some embodiments, R³ is H, and R⁴ is unsubstituted or substituted cycloalkyl. In some embodiments, R³ is H, and R⁴ is substituted cyclohexyl. In some embodiments, R³ is H, and R⁴ is substituted cyclobutyl.

In some embodiments, at least one of R³ and R⁴ is alkyl, alkylene, alkenyl, alkenylene, alkynyl, aryl, heteroaryl, or heterocyclyl, each of which is substituted at least with halo-. In some embodiments, R³ is hydrogen and R⁴ is a ring A. In some embodiments, R⁴ or ring A is cycloalkyl, aryl, heteroaryl, or heterocyclyl, each of which is independently substituted or unsubstituted. In some embodiments, R⁴ or ring A is substituted or unsubstituted aryl. In some embodiments, R⁴ or ring A is substituted or unsubstituted phenyl. In some embodiments, R⁴ or ring A is substituted or unsubstituted cycloalkyl. In some embodiments, R⁴ or ring A is substituted or unsubstituted cyclopropyl. In some embodiments, R⁴ or ring A is substituted cyclopropyl. In some embodiments, R⁴ or ring A is substituted cyclohexyl. In some embodiments, R⁴ or ring A is cyclohexyl substituted with an amino group.

In some embodiments, R³ is H, and R⁴ or ring A is unsubstituted or substituted heterocyclyl. In some embodiments, R⁴ or ring A is heterocyclyl. In some embodiments, R⁴ or ring A is piperidinyl, piperazinyl, tetahydropyranyl, morpholinyl, or pyrrolidinyl, each of which is independently substituted or unsubstituted. In some embodiments, R³ is H, and R⁴ or ring A is substituted piperidinyl. In some embodiments, R³ is H, and R⁴ or ring A is piperidine substituted with alkyl, carboxy, heterocyclyl, or an amide group. In some embodiments, R³ is H, and R⁴ or ring A is unsubstituted or substituted methyl piperidinyl. In some embodiments, R³ is H, and R⁴ or ring A is 3-fluoro-1-methylpiperidinyl. In some embodiments, R³ is H, and R⁴ or ring A is piperidinyl substituted with methoxypropanol. In some embodiments, R³ is H, and R⁴ or ring A is 3-fluoro-1-(2-hydroxy-3-methoxypropyl)piperidinyl. In some embodiments, R³ is H, and R⁴ or ring A is unsubstituted or substituted tetrahydropyranyl. In some embodiments, R³ is H, and R⁴ or ring A is unsubstituted tetrahydropyranyl. In some embodiments, R³ is H, and R⁴ or ring A is tetrahydropyranyl substituted with alkyl. In some embodiments, R³ is H, and R⁴ or ring A is tetrahydrothiopyran-1,1-diooxide.

In some embodiments, R⁴ or ring A is cycloalkyl, aryl, heteroaryl, or heterocyclyl, each of which is substituted at least with halo-. In some embodiments, R⁴ or ring A is C₄-C₆-cycloalkyl substituted with at least halo-. In some embodiments, R⁴ or ring A is cyclohexyl substituted with at least halo-. In some embodiments, R⁴ or ring A is aryl substituted with at least halo-. In some embodiments, R⁴ or ring A is phenyl substituted with at least halo-. In some embodiments, R⁴ or ring A is aryl substituted with fluoro-. In some embodiments, R⁴ or ring A is phenyl substituted with fluoro-. In some embodiments, R⁴ or ring A is aryl substituted with chloro-. In some embodiments, R⁴ or ring A is phenyl substituted with chloro-. In some embodiments, R⁴ or ring A is heteroaryl substituted with at least halo-. In some embodiments, R⁴ or ring A is heteroaryl substituted with fluoro-. In some embodiments, R⁴ or ring A is heteroaryl substituted with chloro-. In some embodiments, R⁴ or ring A is C₄-C₆-heterocyclyl substituted with at least halo-. In some embodiments, R⁴ or ring A is heterocyclyl substituted with fluoro-. In some embodiments, R⁴ or ring A is heterocyclyl substituted with chloro-.

In some embodiments, R⁴ or ring A is piperidinyl, piperazinyl, tetahydropyranyl, morpholinyl, or pyrrolidinyl, each of which is independently substituted with at least halo-. In some embodiments, R⁴ or ring A is piperidinyl substituted with halo-. In some embodiments, R⁴ or ring A is methylpiperidinyl substituted with halo-. In some embodiments, R⁴ or ring A is 3-fluoro-1-methylpiperidinyl. In some embodiments, R⁴ or ring A is 3-fluoro-1-(2-hydroxy-3-methoxypropyl)piperidinyl. In some embodiments, R⁴ or ring A is tetrahydropyranyl substituted with at least halo-.

In some embodiments, R⁴ or Ring A is a ring that is:

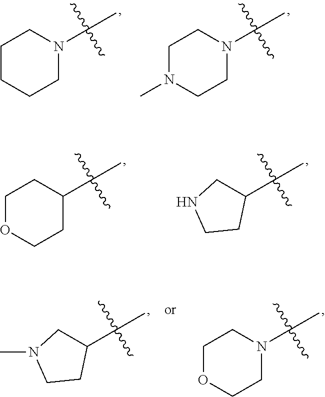

wherein the ring is substituted or unsubstituted. In some embodiments, the ring is substituted with halo-. In some embodiments, the ring is substituted with fluoro. In some embodiments, R³ is H, and R⁴ is a ring that is

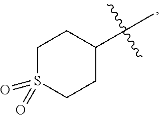 [SMILES: *C1CCS(=O)(=O)CC1]

wherein the ring is substituted or unsubstituted. In some embodiments, the ring is substituted with halo-. In some embodiments, the ring is substituted with fluoro. In some embodiments, R³ is H, and R⁴ is a ring that is

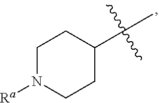

wherein the ring is substituted or unsubstituted. In some embodiments, R^{a} is alkylene. In some embodiments, R^{a} is methyl. In some embodiments, the ring is substituted with halo. In some embodiments, the ring is substituted with fluoro. In some embodiments, R³ is H, and R⁴ is a ring that is

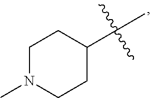 [SMILES: *C1CCN(C)CC1]

wherein the ring is substituted or unsubstituted. In some embodiments, the ring is substituted with halo. In some embodiments, the ring is substituted with fluoro. In some embodiments, R³ is H, and R⁴ is a ring that is

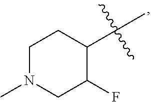 [SMILES: *C1CCN(C)CC1F]

wherein the ring is substituted or unsubstituted.

In some embodiments, the R⁴ or ring A is substituted with one or more substituents selected from a hydroxyl group, sulfhydryl group, halogens, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R³ and R⁴ together with the nitrogen atom to which R³ and R⁴ are bound form a ring, wherein the ring is substituted or unsubstituted. In some embodiments, R³ and R⁴ together with the nitrogen atom to which R³ and R⁴ are bound form a substituted heterocycle. In some embodiments, R³ and R⁴ together with the nitrogen atom to which R³ and R⁴ are bound form a heterocycle substituted with a hydroxyl group, halogen, amino group, or alkyl group. In some embodiments, R³ and R⁴ together with the nitrogen atom to which R³ and R⁴ are bound form a heterocycle, wherein the heterocycle is substituted by a substituted or unsubstituted heterocycle.

In some embodiments, R³ and R⁴ together with the nitrogen atom to which R³ and R⁴ are bound form a ring of a following formula:

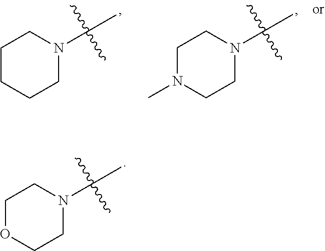

In some embodiments, the compound is of the formula:

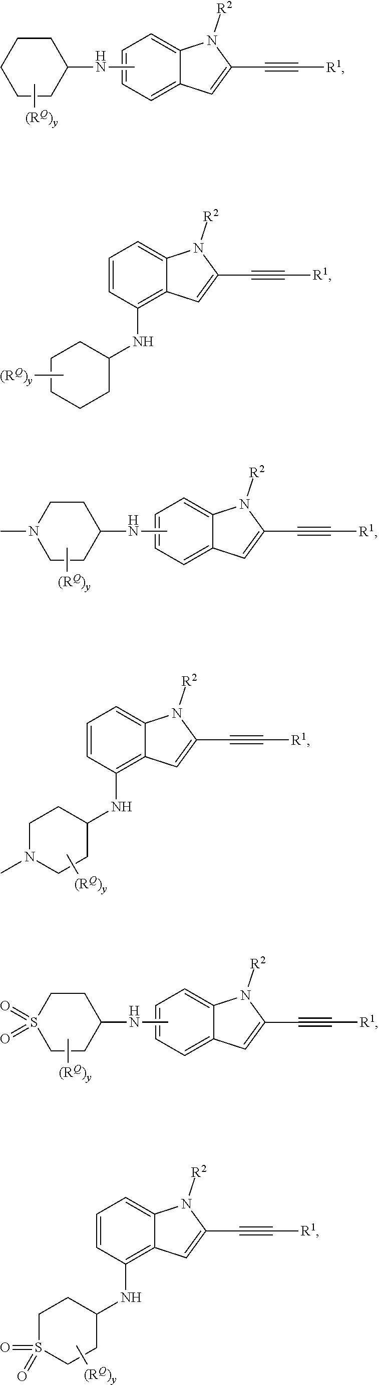

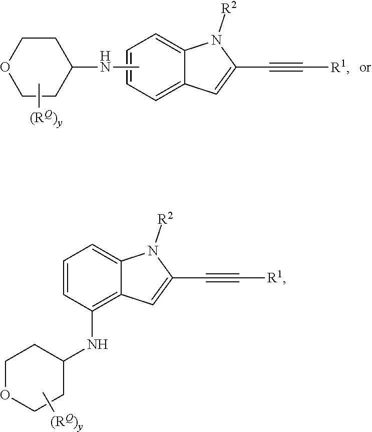

wherein:
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, or hydrogen;
each R^{Q} is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
y is 0, 1, 2, 3, or 4;
each R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen.

In some embodiments, R¹ is substituted C₁-C₃-alkyl. In some embodiments, R¹ is C₁-C₃-alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group. In some embodiments, R¹ is methyl substituted with NR¹⁶R¹⁷, wherein R¹⁶ is hydrogen, and R¹⁷ is substituted aryl. In some embodiments, R¹ is methyl substituted with NR¹⁶R¹⁷, wherein R¹⁶ is hydrogen, and R¹⁷ is substituted phenyl. In some embodiments, R¹ is methyl substituted with NR¹⁶R¹⁷, wherein R¹⁶ is hydrogen, and R¹⁷ is phenyl, substituted with a sulfoxide group, carboxyl group, amide group, amino group, alkyl, alkoxy, hydroxy, halo, cyano, or heterocyclyl, each of which is independently substituted or unsubstituted. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is a substituted amide group. In some embodiments, R¹⁷ is substituted with methoxy and sulfonamide.

In some embodiments, R² is hydrogen or alkyl. In some embodiments, R² is substituted C₁-C₅-alkylene. In some embodiments, R² is trifluoroethyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen.

In some embodiments, the compound is of the formula:

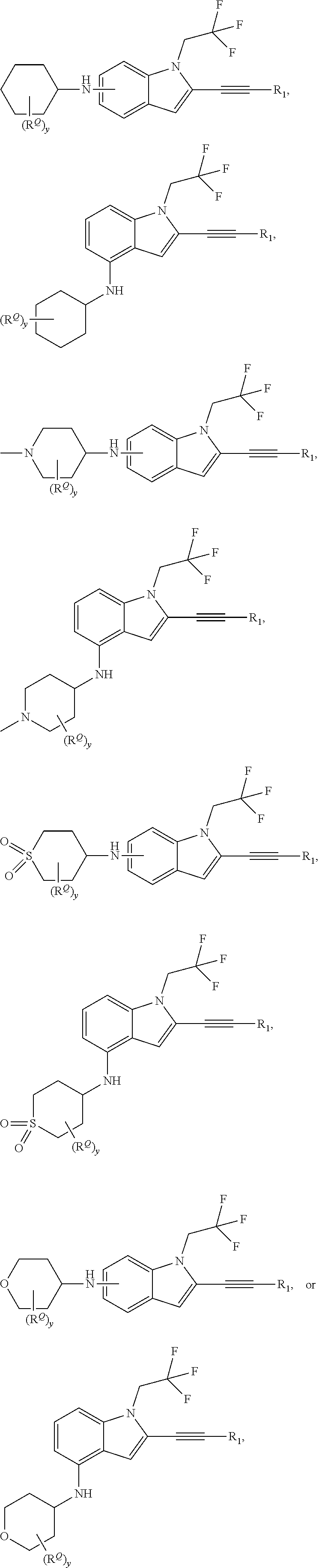

or a pharmaceutically-acceptable salt thereof, wherein the variables are as defined above.

In some embodiments, each R^{Q} is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen. In some embodiments, each R^{Q} is

In some embodiments, y is 1. In some embodiments, y is 2. In some embodiments, y is 3. In some embodiments, y is 4.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen.

In some embodiments, R¹ is substituted alkyl. In some embodiments, R¹ is substituted C₁-C₃-alkyl. In some embodiments, R¹ is alkyl substituted with NR¹⁶R¹⁷. In some embodiments, R¹ is C₁-C₃-alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, R¹⁶ is alkyl, alkenyl, aryl, heteroaryl, heterocyclyl, or hydrogen, and R¹⁷ is aryl, heteroaryl, or heterocyclyl. In some embodiments, R¹⁶ is hydrogen, and R¹⁷ is phenyl, indolyl, piperidinyl, imidazolyl, thiazolyl, morpholinyl, pyrrolyl, or pyridinyl, each of which is substituted or unsubstituted.

In some embodiments, the compound is of the formula:

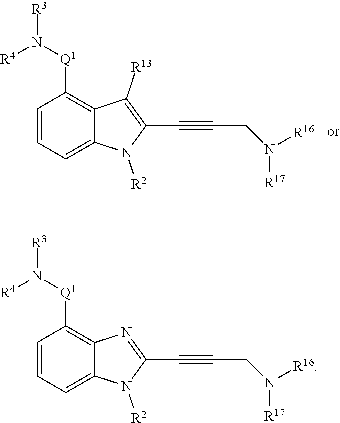

In some embodiments, the compound is of the formula:

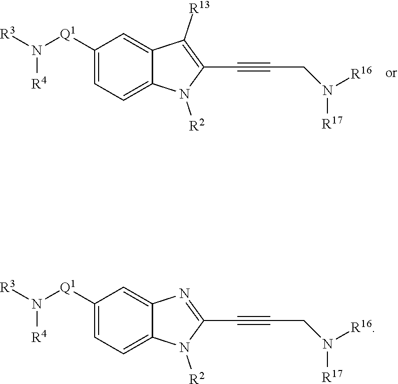

In some embodiments, the compound is of the formula:

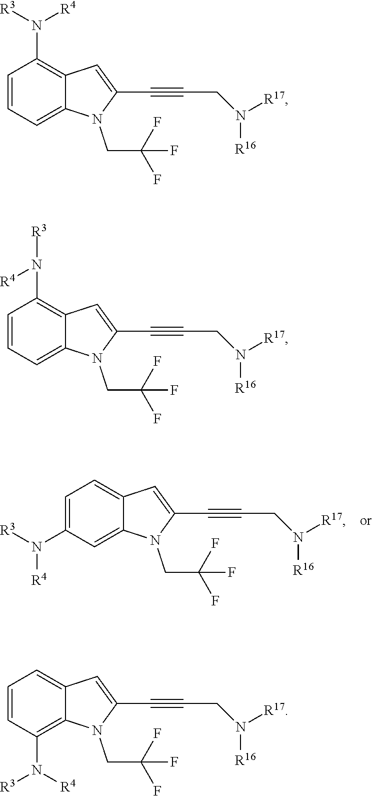

In some embodiments, each R¹⁶ and R¹⁷ is independently alkyl, alkenyl, aryl, heteroaryl, heterocyclyl, or hydrogen. In some embodiments, R¹⁶ is aryl, and R¹⁷ is alkyl. In some embodiments, R¹⁶ is aryl, and R¹⁷ is hydrogen. In some embodiments, R¹⁶ is heteroaryl, and R¹⁷ is alkyl. In some embodiments, R¹⁶ is heteroaryl, and R¹⁷ is hydrogen. In some embodiments, R¹⁶ is substituted heteroaryl, and R¹⁷ is hydrogen. In some embodiments, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁶ is hydrogen. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano. In some embodiments, R¹⁶ is alkyl, alkenyl, aryl, heteroaryl, heterocyclyl, or hydrogen, and R¹⁷ is aryl, heteroaryl, or heterocyclyl. In some embodiments, R¹⁶ is hydrogen, and R¹⁷ is phenyl, indolyl, piperidinyl, imidazolyl, thiazolyl, morpholinyl, pyrrolyl, or pyridinyl, each of which is substituted or unsubstituted. In some embodiments, R¹⁶ is hydrogen, and R¹⁷ is substituted phenyl. In some embodiments, R¹⁶ is hydrogen, and R¹⁷ is phenyl substituted with a sulfoxide group, carboxyl group, amide group, amino group, alkyl, alkoxy, hydroxy, halo, cyano, or heterocyclyl, each of which is independently substituted or unsubstituted. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is a substituted amide group. In some embodiments, R¹⁷ is substituted with methoxy and sulfonamide.

In some embodiments, each R³ and R⁴ is independently unsubstituted or substituted alkyl. In some embodiments, R³ is hydrogen and R⁴ is —C(O)R¹⁹, —C(O)OR¹⁹, alkyl, alkenyl, alkynyl, aryl, heteroaryl, or heterocyclyl, each of which is independently substituted or unsubstituted. In some embodiments, R³ is hydrogen, and R⁴ is alkyl, aryl, heteroaryl, or heterocyclyl, each of which is independently substituted or unsubstituted. In some embodiments, R³ is H, and R⁴ is substituted heterocyclyl. In some embodiments, R³ is H, and R⁴ is substituted or unsubstituted C₄-C₆-heterocyclyl. In some embodiments, R³ is H, and R⁴ is substituted alkyl. In some embodiments, R³ is H, and R⁴ is substituted C₁-C₆-alkyl. In some embodiments, R³ is H, and R⁴ is substituted or unsubstituted cycloalkyl. In some embodiments, R³ is H, and R⁴ is substituted or unsubstituted C₄-C₆-cycloalkyl. In some embodiments, R³ is H, and R⁴ is C₄-C₆-cycloalkyl substituted with an amino group.

In some embodiments, the compound is of the formula:

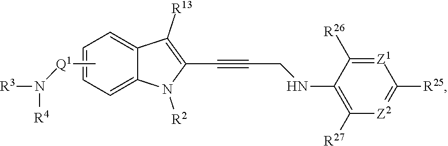

wherein:
Q¹ is C═O, C═S, C═CR¹⁴R¹⁵, C═NR¹⁴, alkylene, alkenylene, or alkynylene, each of which is independently substituted or unsubstituted, or a bond;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, or hydrogen;
each R³ and R⁴ is independently, —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each Z¹ and Z² is independently CR²⁸, CR²⁹, or N;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen;
each R²³ and R²⁴ is independently alkyl, alkenyl, alkynyl, aryl, heteroaryl, or heterocyclyl, each of which is independently substituted or unsubstituted, or hydrogen; and
each R²⁵, R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.
   or a pharmaceutically-acceptable salt thereof.

In some embodiments, Z¹ is N. In some embodiments, Z¹ and Z² are N. In some embodiments, each R²⁵ and R²⁶ is independently a halogen. In some embodiments, R²⁵ is

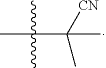 [SMILES: *C(C)(C)C#N]

In some embodiments, R²⁵ is a substituted sulfone group. In some embodiments, R²⁵ is a sulfone group substituted with alkyl. In some embodiments, R²⁵ is a methanesulfonyl group. In some embodiments, R²⁵ is a sulfone group substituted with an amino group. In some embodiments, R²⁵ is a sulfonamide. In some embodiments, R²⁵ is a carboxy group. In some embodiments, R²⁵ is a methoxycarbonyl group.

In some embodiments, the compound is of the formula:

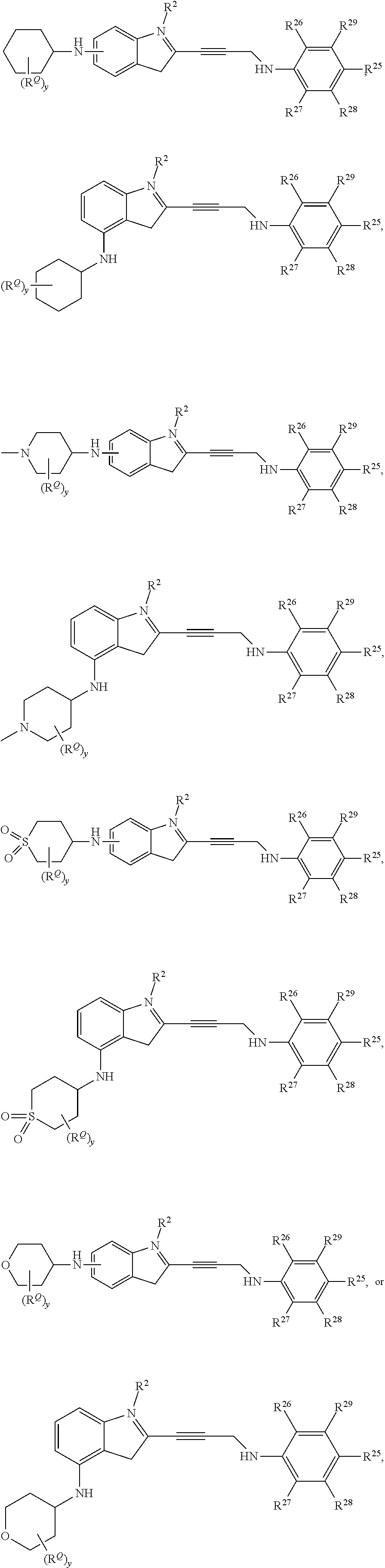

wherein:
R² is —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R^{Q} is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted;
y is 0, 1, 2, 3, or 4;
each R²¹ and R²² is independently alkyl, alkenyl, alkynyl, aryl, heteroaryl, or heterocyclyl, each of which is independently substituted or unsubstituted, or hydrogen; and
each R²⁵, R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.
   or a pharmaceutically-acceptable salt thereof.

In some embodiments, the compound is of the formula:

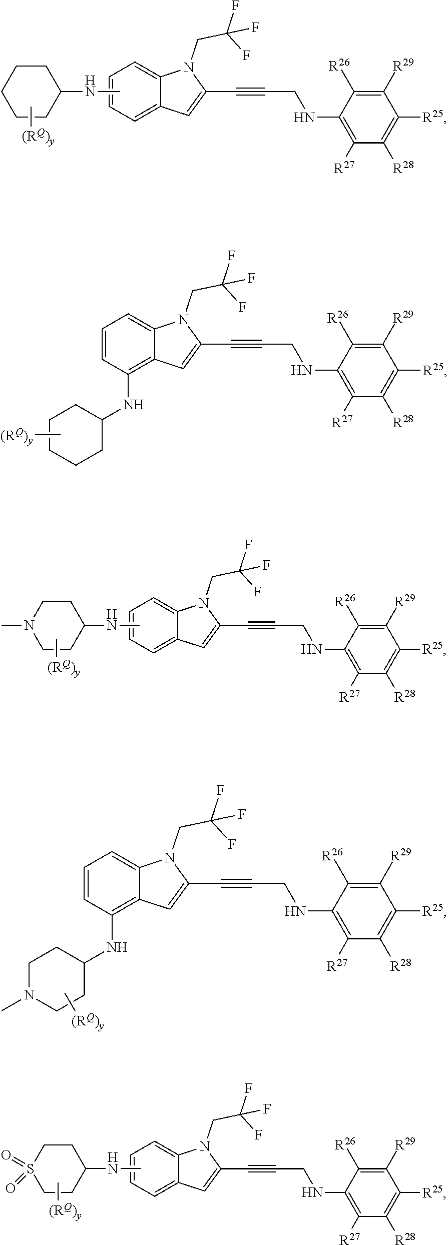

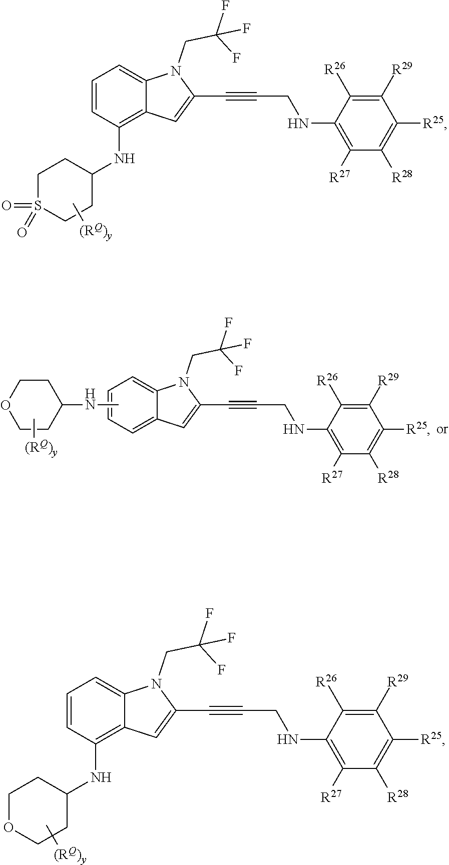

In some embodiments, R²⁵ is a substituted sulfone group. In some embodiments, R²⁵ is a sulfone group substituted with alkyl. In some embodiments, R²⁵ is a methanesulfonyl group. In some embodiments, R²⁵ is a sulfone group substituted with an amino group. In some embodiments, R²⁵ is a sulfonamide. In some embodiments, R²⁵ is a carboxy group. In some embodiments, R²⁵ is a methoxycarbonyl group.

In some embodiments, the compound is of the formula:

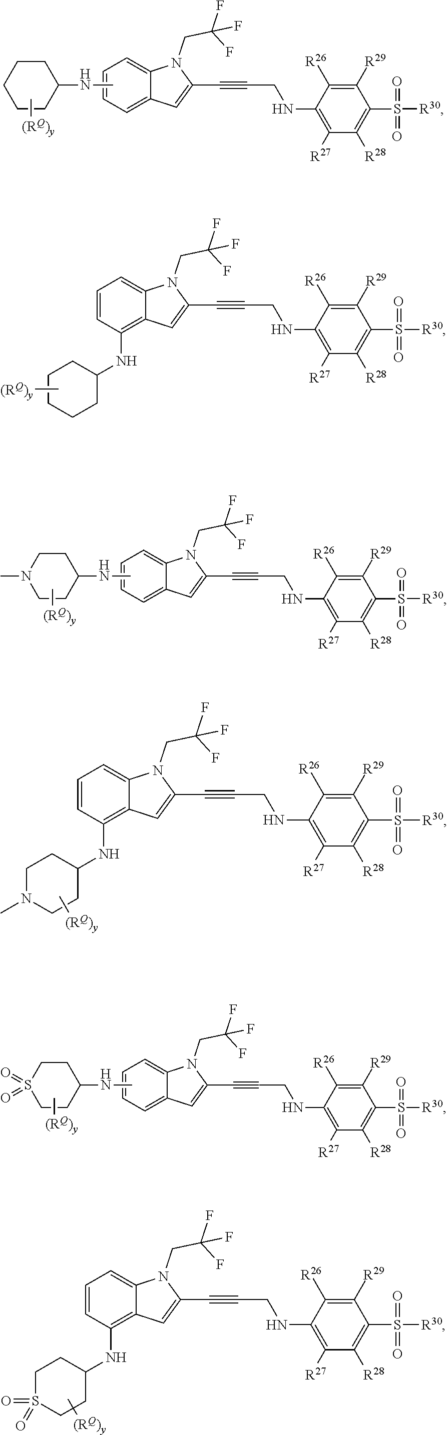

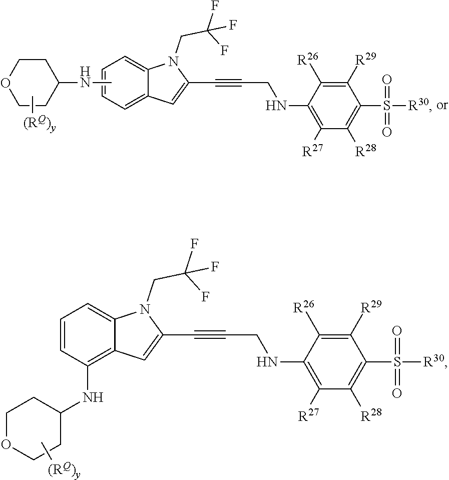

wherein:
each R^{Q} is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted;
y is 0, 1, 2, 3, or 4;
each R²¹ and R²² is independently alkyl, alkenyl, alkynyl, aryl, heteroaryl, or heterocyclyl, each of which is independently substituted or unsubstituted, or hydrogen;
each R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group; and
R³⁰ is alkyl or an amino group, each of which is substituted or unsubstituted,
   or a pharmaceutically-acceptable salt thereof.

In some embodiments, R³⁰ is methyl. In some embodiments, R³⁰ is NH₂. In some embodiments, R³⁰ is NHMe. In some embodiments, R³⁰ is NMe₂.

In some embodiments, the compound is of the formula:

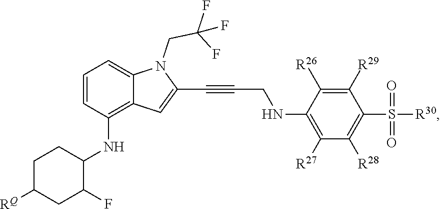

wherein R³⁰ is alkyl or an amino group, each of which is unsubstituted or substituted. In some embodiments, R³⁰ is methyl.

Non-limiting examples of compounds of the current disclosure include the following:

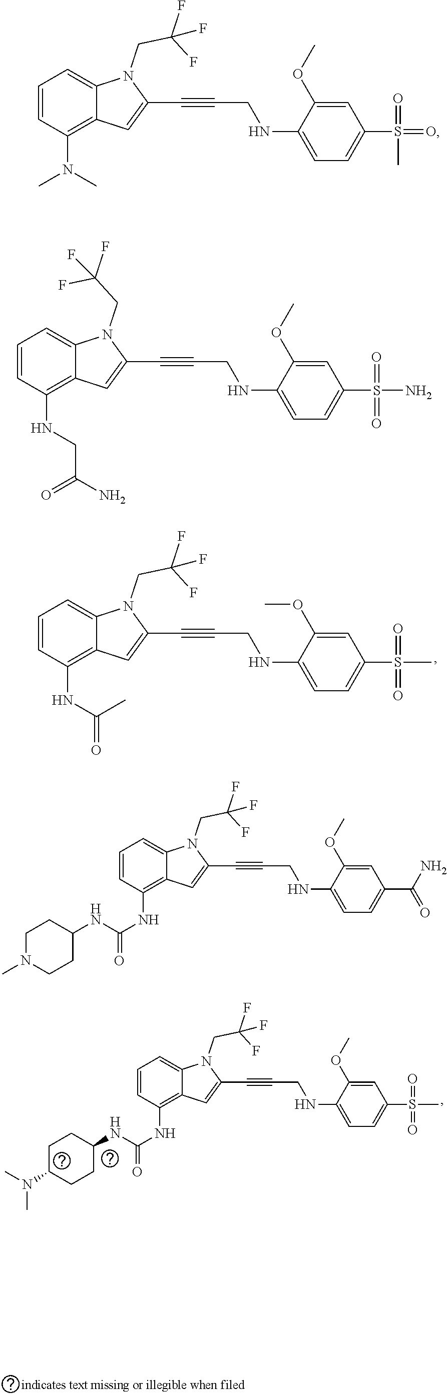

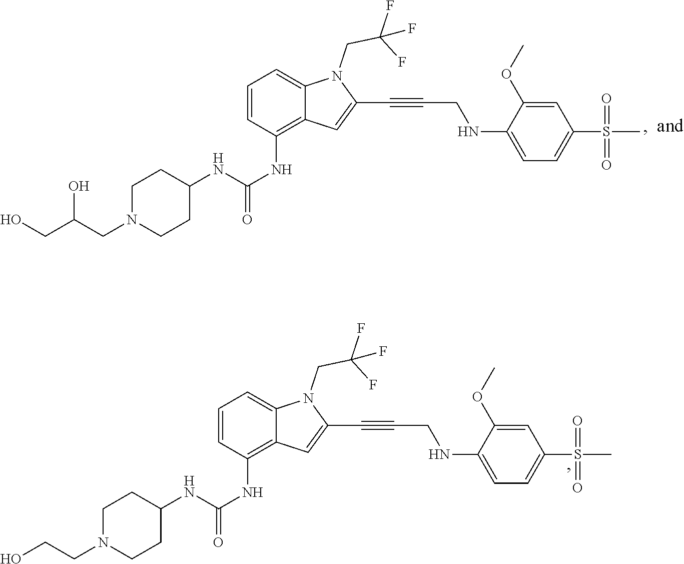 [SMILES: COc1cc(S(C)(=O)=O)ccc1NCC#Cc1cc2c(NC(=O)NC3CCN(CC(O)CO)CC3)cccc2n1CC(F)(F)F ; COc1cc(S(C)(=O)=O)ccc1NCC#Cc1cc2c(NC(=O)NC3CCN(CCO)CC3)cccc2n1CC(F)(F)F]

or a pharmaceutically-acceptable salt thereof.

Non-limiting examples of compounds of the current disclosure include the following:

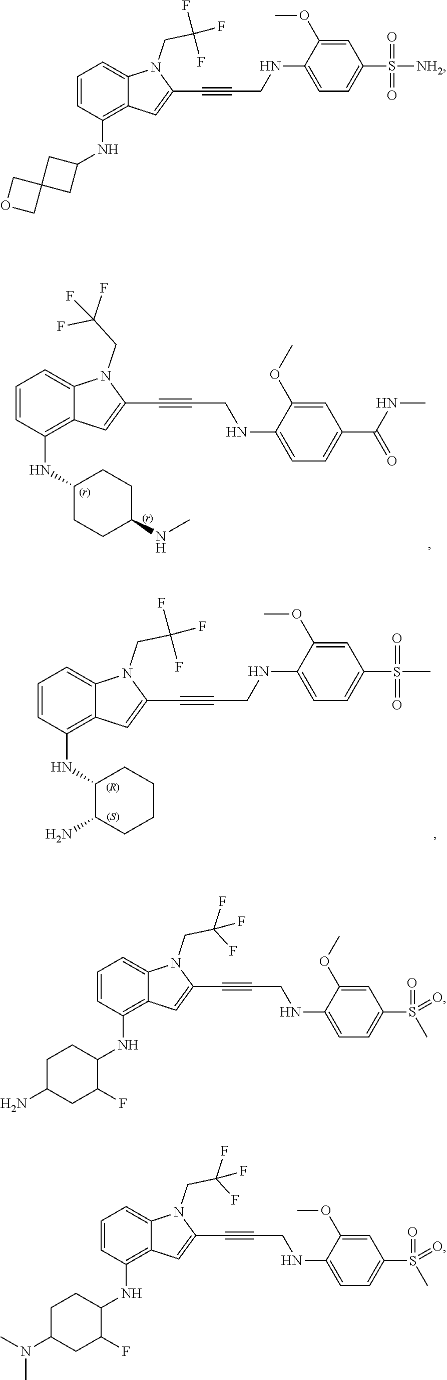

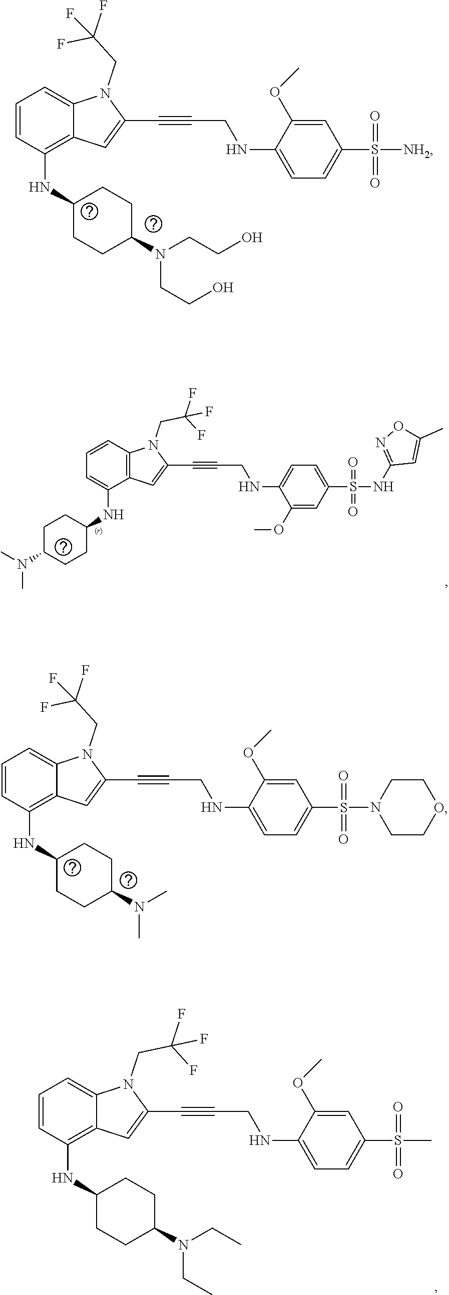

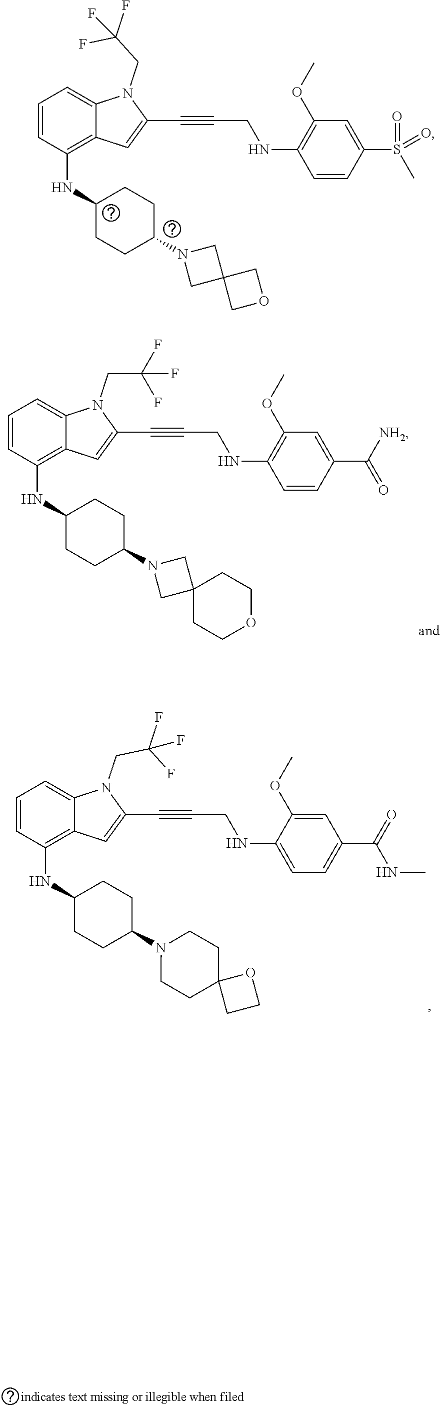 [SMILES: COc1cc(S(C)(=O)=O)ccc1NCC#Cc1cc2c(N[C@H]3CC[C@H](N4CC5(COC5)C4)CC3)cccc2n1CC(F)(F)F ; COc1cc(C(N)=O)ccc1NCC#Cc1cc2c(N[C@H]3CC[C@@H](N4CC5(CCOCC5)C4)CC3)cccc2n1CC(F)(F)F ; CNC(=O)c1ccc(NCC#Cc2cc3c(N[C@H]4CC[C@@H](N5CCC6(CCO6)CC5)CC4)cccc3n2CC(F)(F)F)c(OC)c1]

or a pharmaceutically-acceptable salt thereof.

Non-limiting examples of compounds of the current disclosure include the following:

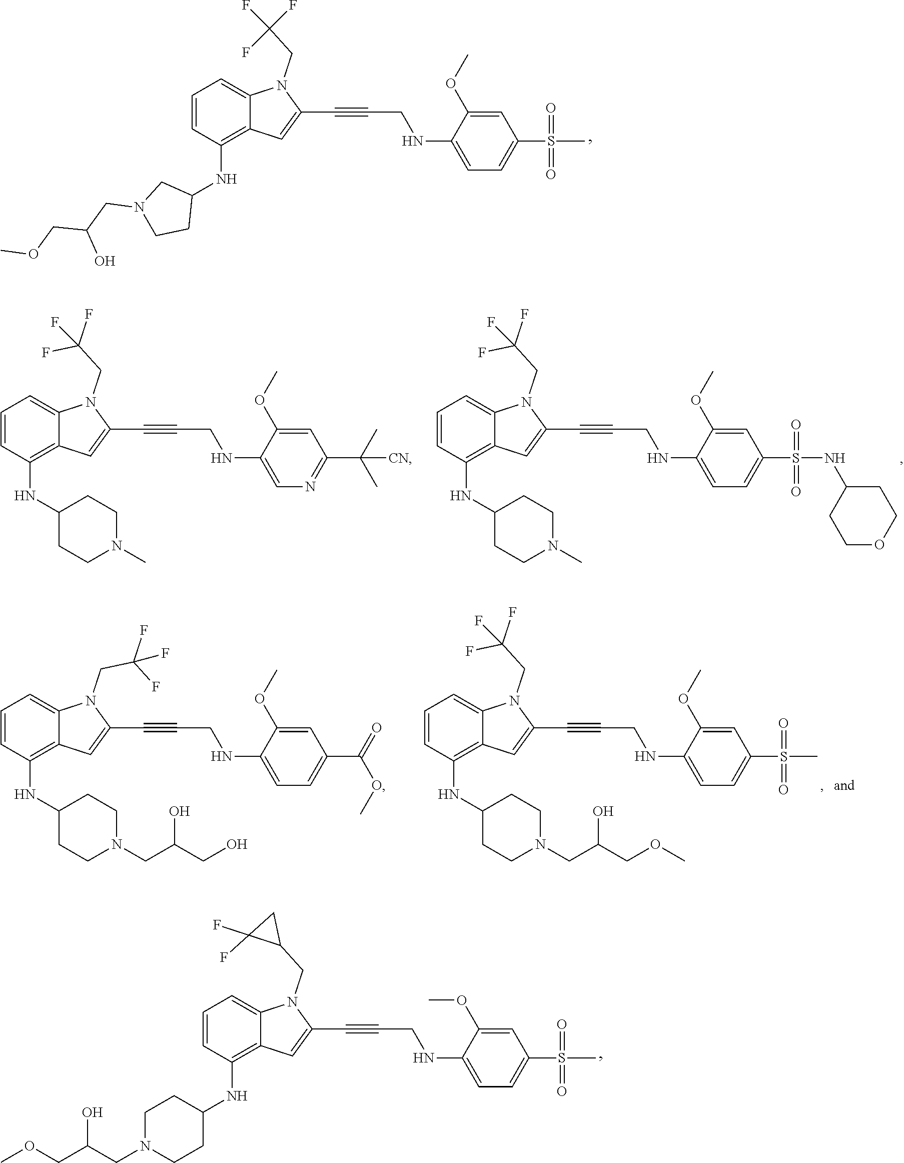

or a pharmaceutically-acceptable salt thereof.

Non-limiting examples of compounds of the current disclosure include the following:

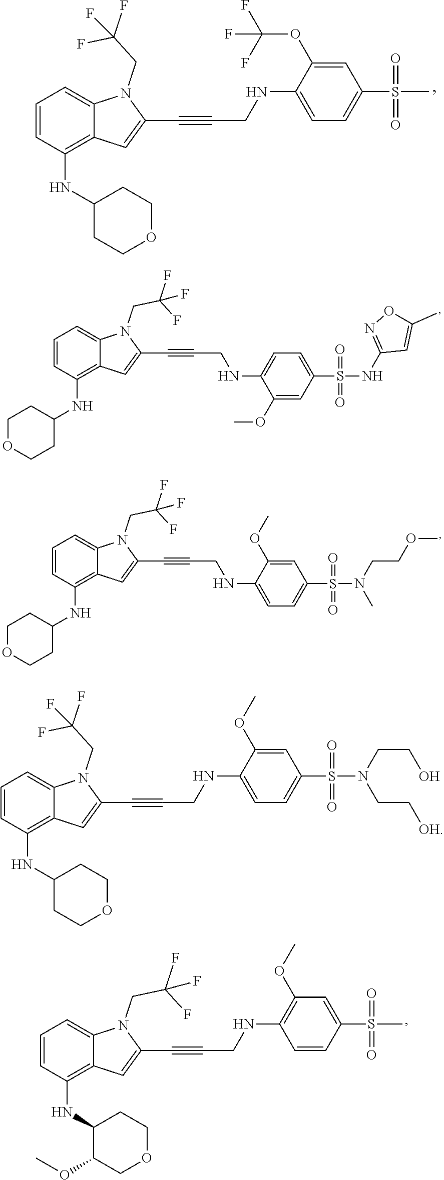

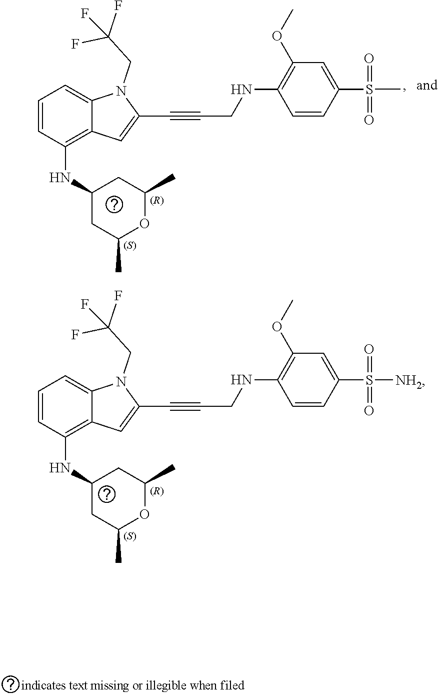

or a pharmaceutically-acceptable salt thereof.

Non-limiting examples of compounds of the current disclosure include the following:

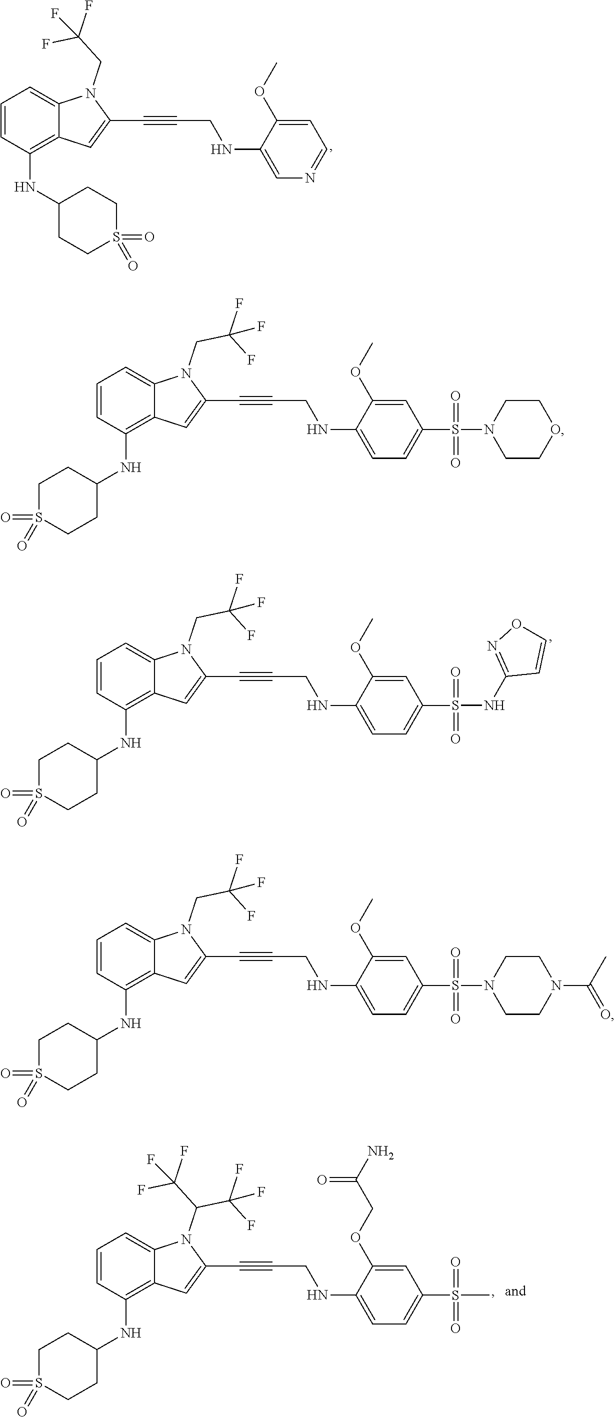 [SMILES: COc1ccncc1NCC#Cc1cc2c(NC3CCS(=O)(=O)CC3)cccc2n1CC(F)(F)F ; COc1cc(S(=O)(=O)N2CCOCC2)ccc1NCC#Cc1cc2c(NC3CCS(=O)(=O)CC3)cccc2n1CC(F)(F)F ; COc1cc(S(=O)(=O)Nc2ccon2)ccc1NCC#Cc1cc2c(NC3CCS(=O)(=O)CC3)cccc2n1CC(F)(F)F ; COc1cc(S(=O)(=O)N2CCN(C(C)=O)CC2)ccc1NCC#Cc1cc2c(NC3CCS(=O)(=O)CC3)cccc2n1CC(F)(F)F ; CS(=O)(=O)c1ccc(NCC#Cc2cc3c(NC4CCS(=O)(=O)CC4)cccc3n2C(C(F)(F)F)C(F)(F)F)c(OCC(N)=O)c1]

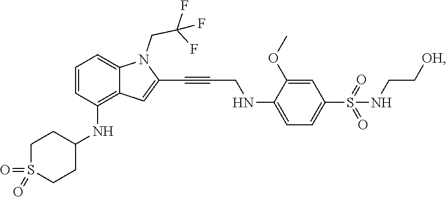 [SMILES: COc1cc(S(=O)(=O)NCCO)ccc1NCC#Cc1cc2c(NC3CCS(=O)(=O)CC3)cccc2n1CC(F)(F)F]

or a pharmaceutically-acceptable salt thereof.

Non-limiting examples of compounds of the current disclosure include the following:

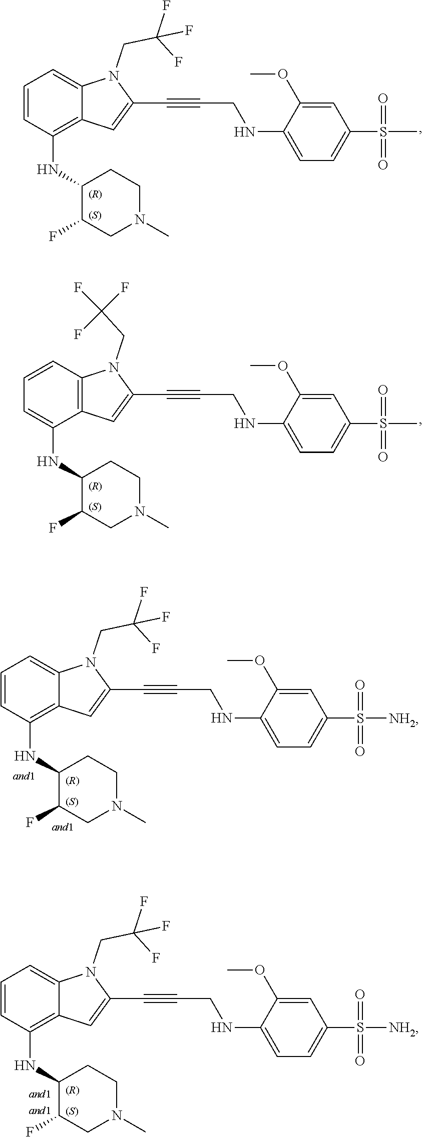

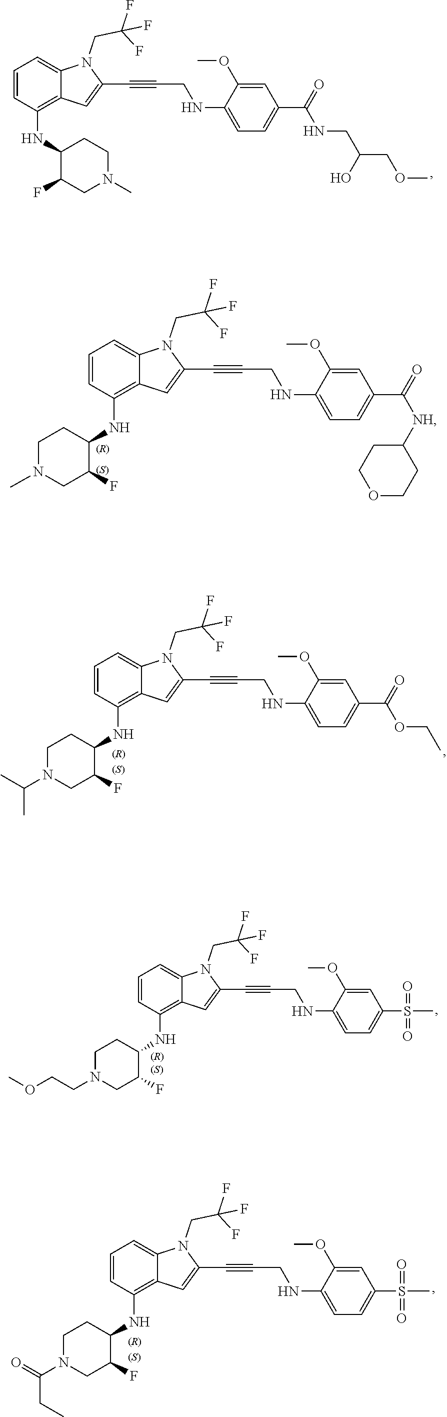

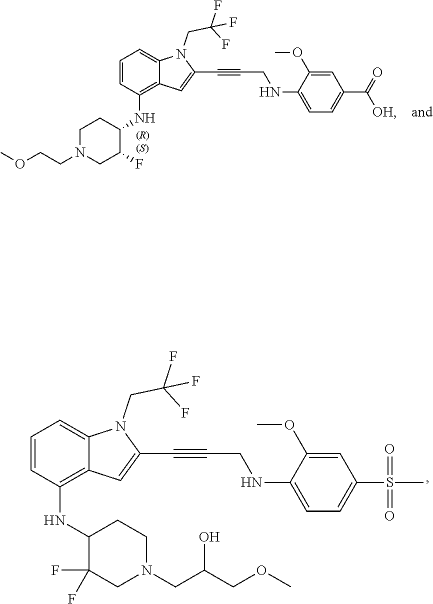

or a pharmaceutically-acceptable salt thereof.

Non-limiting examples of compounds of the current disclosure include the following:

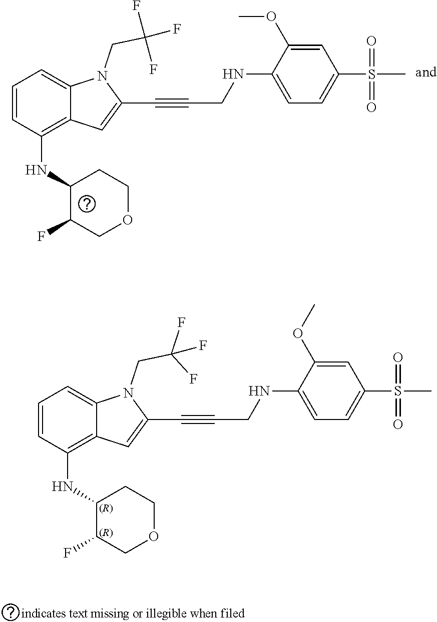

or a pharmaceutically-acceptable salt thereof.

Non-limiting examples of compounds of the current disclosure include the following:

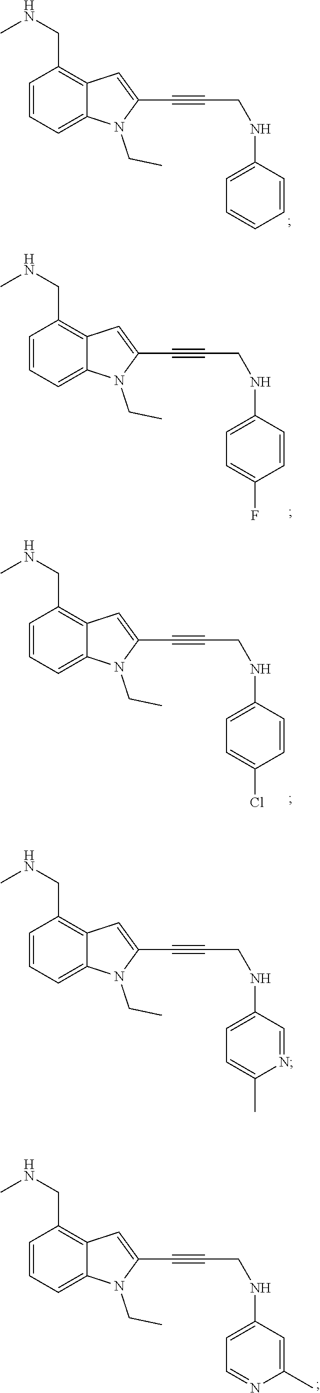

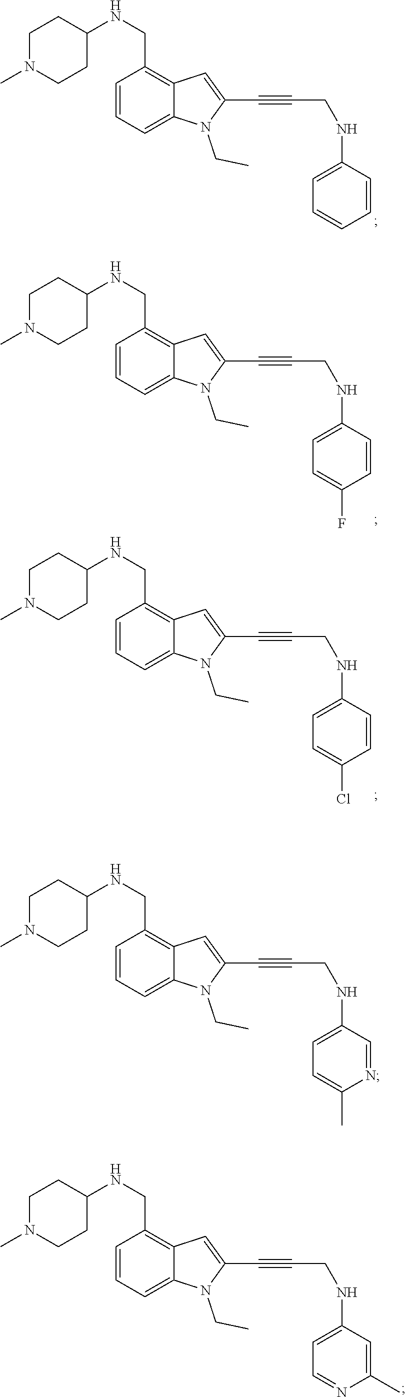

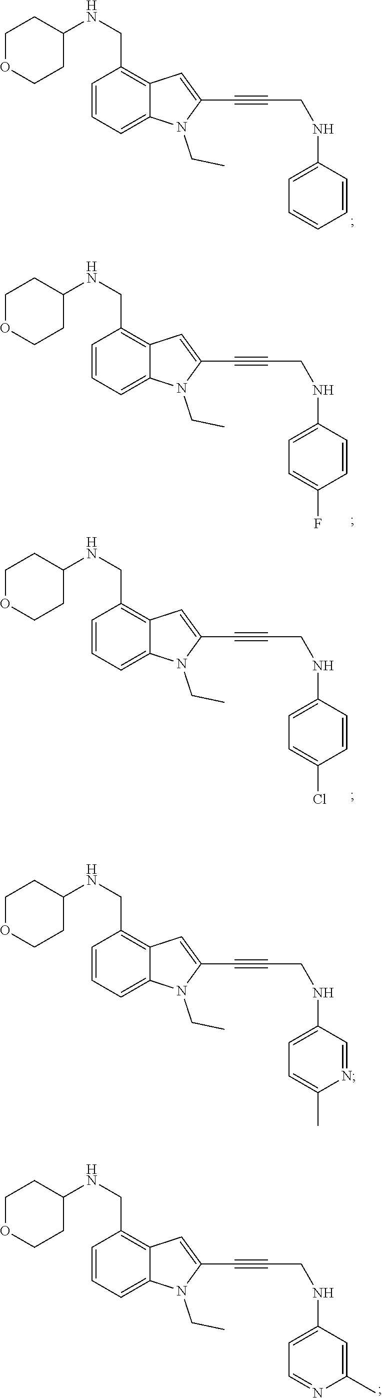 [SMILES: CCn1c(C#CCNc2ccccc2)cc2c(CNC3CCOCC3)cccc21 ; CCn1c(C#CCNc2ccc(F)cc2)cc2c(CNC3CCOCC3)cccc21 ; CCn1c(C#CCNc2ccc(Cl)cc2)cc2c(CNC3CCOCC3)cccc21 ; CCn1c(C#CCNc2ccc(C)nc2)cc2c(CNC3CCOCC3)cccc21 ; CCn1c(C#CCNc2ccnc(C)c2)cc2c(CNC3CCOCC3)cccc21]

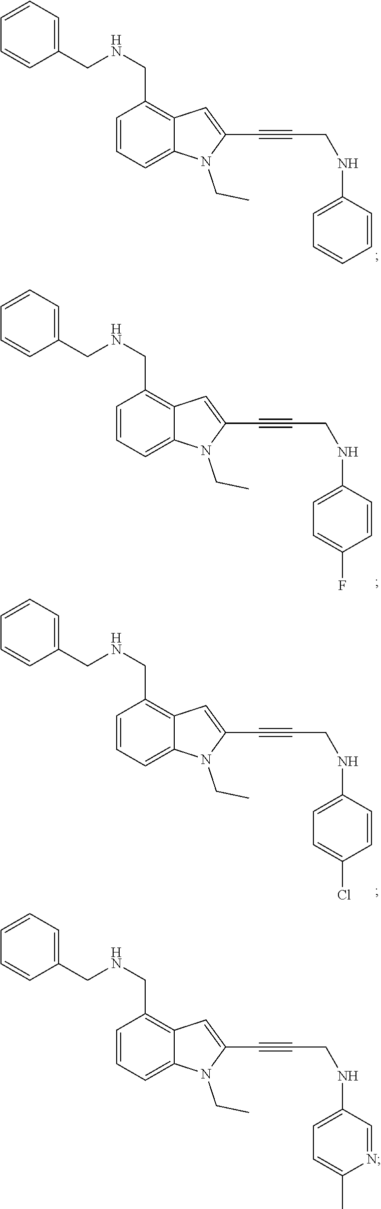 [SMILES: CCn1c(C#CCNc2ccccc2)cc2c(CNCc3ccccc3)cccc21 ; CCn1c(C#CCNc2ccc(F)cc2)cc2c(CNCc3ccccc3)cccc21 ; CCn1c(C#CCNc2ccc(Cl)cc2)cc2c(CNCc3ccccc3)cccc21 ; CCn1c(C#CCNc2ccc(C)nc2)cc2c(CNCc3ccccc3)cccc21]

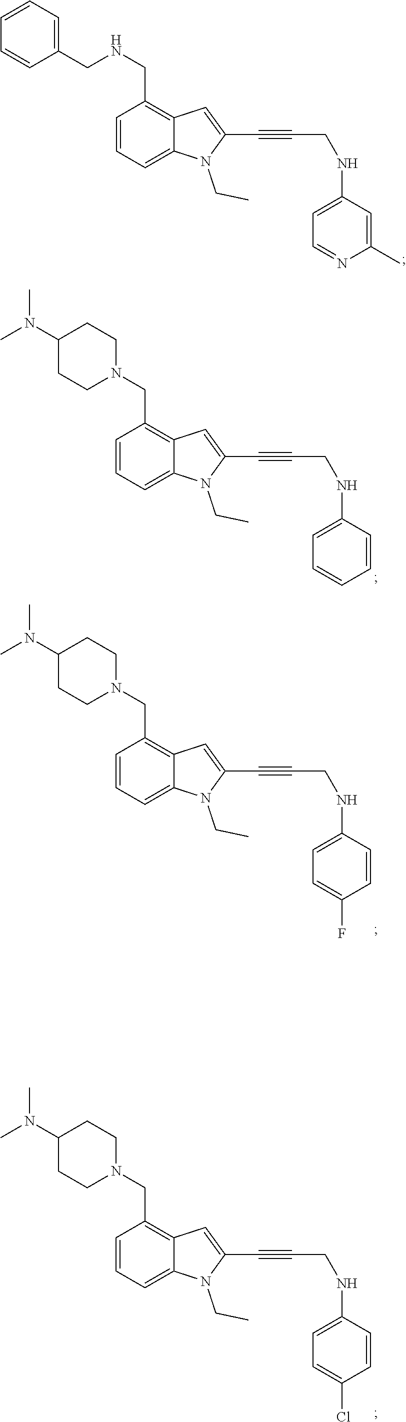

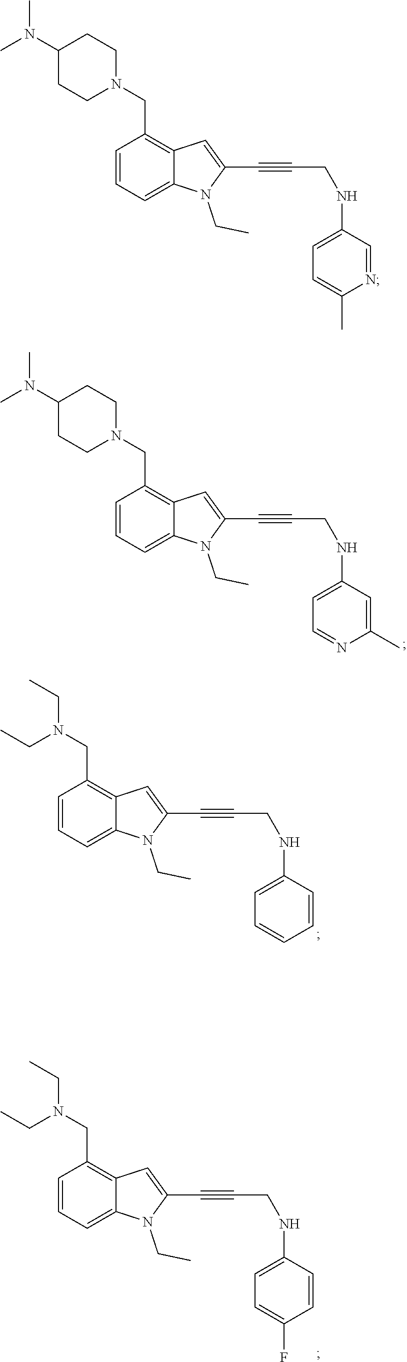

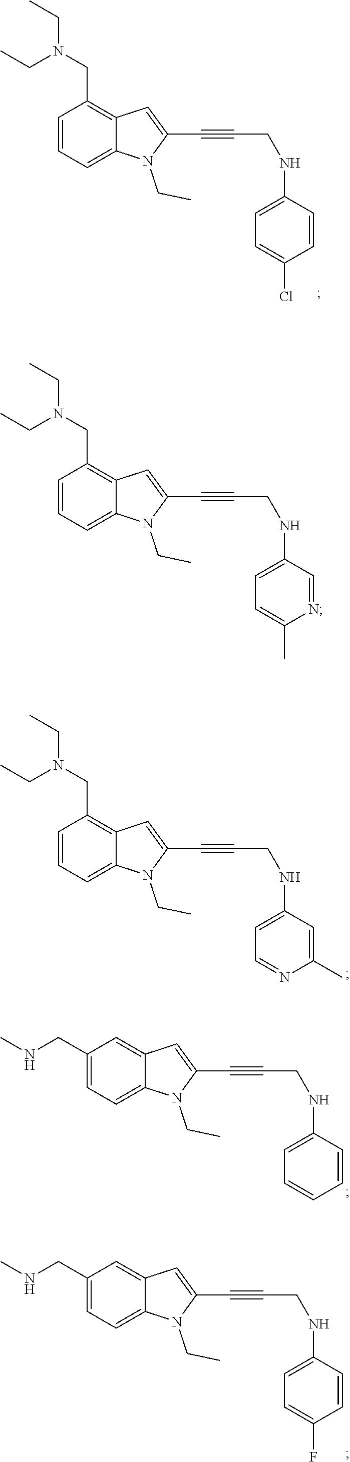

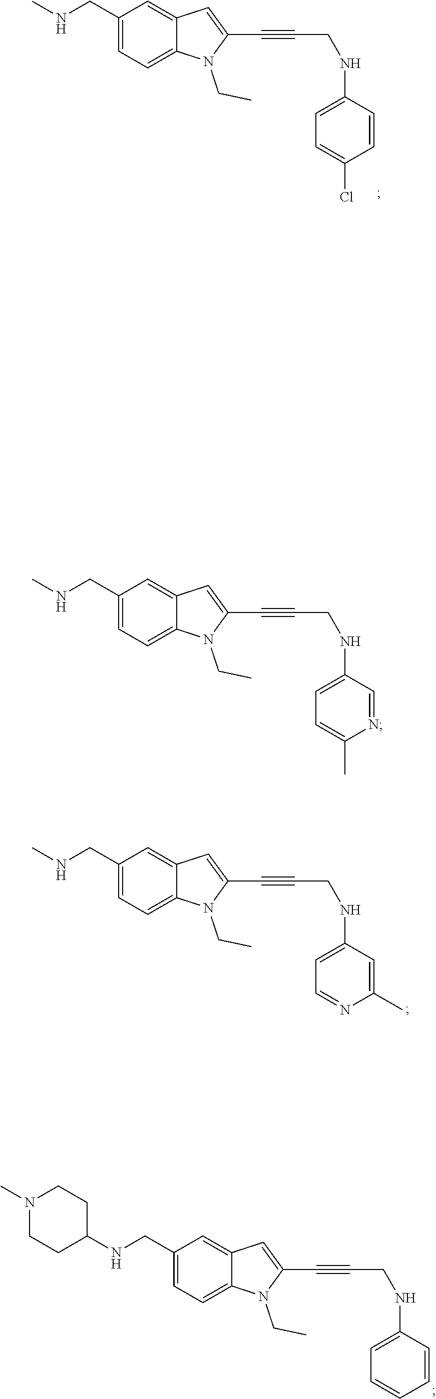

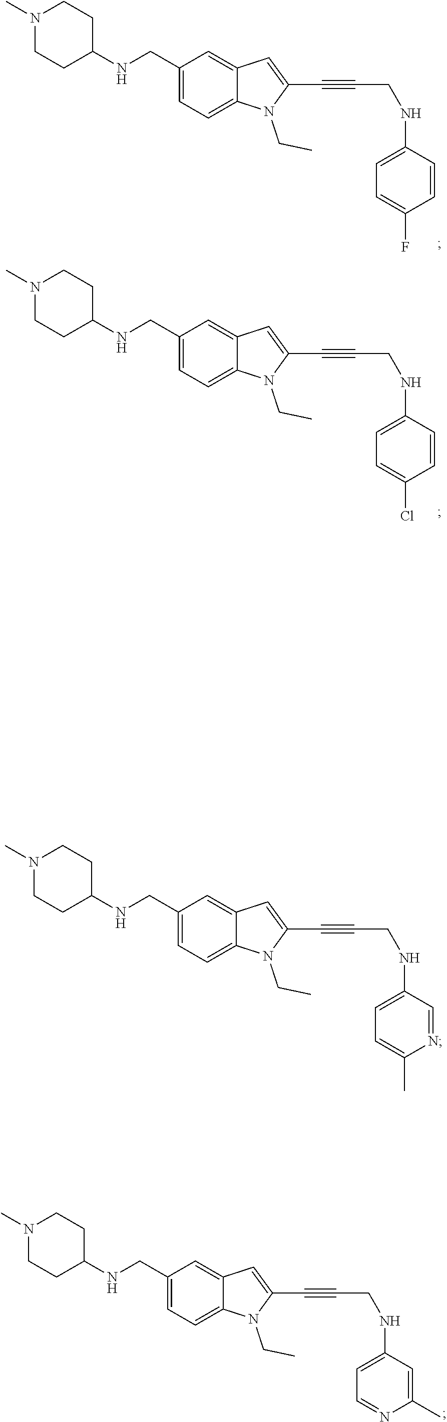

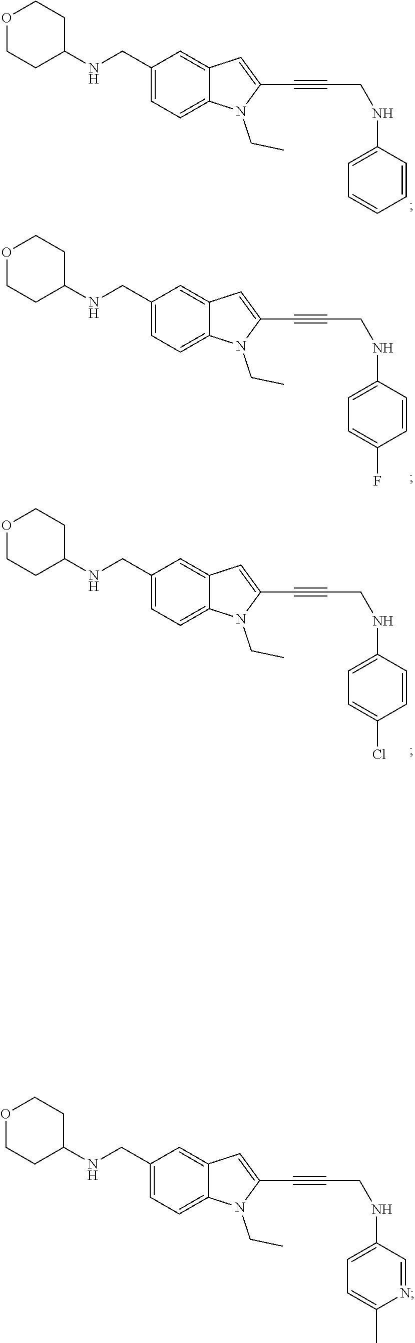 [SMILES: CCn1c(C#CCNc2ccccc2)cc2cc(CNC3CCOCC3)ccc21 ; CCn1c(C#CCNc2ccc(F)cc2)cc2cc(CNC3CCOCC3)ccc21 ; CCn1c(C#CCNc2ccc(Cl)cc2)cc2cc(CNC3CCOCC3)ccc21 ; CCn1c(C#CCNc2ccc(C)nc2)cc2cc(CNC3CCOCC3)ccc21]

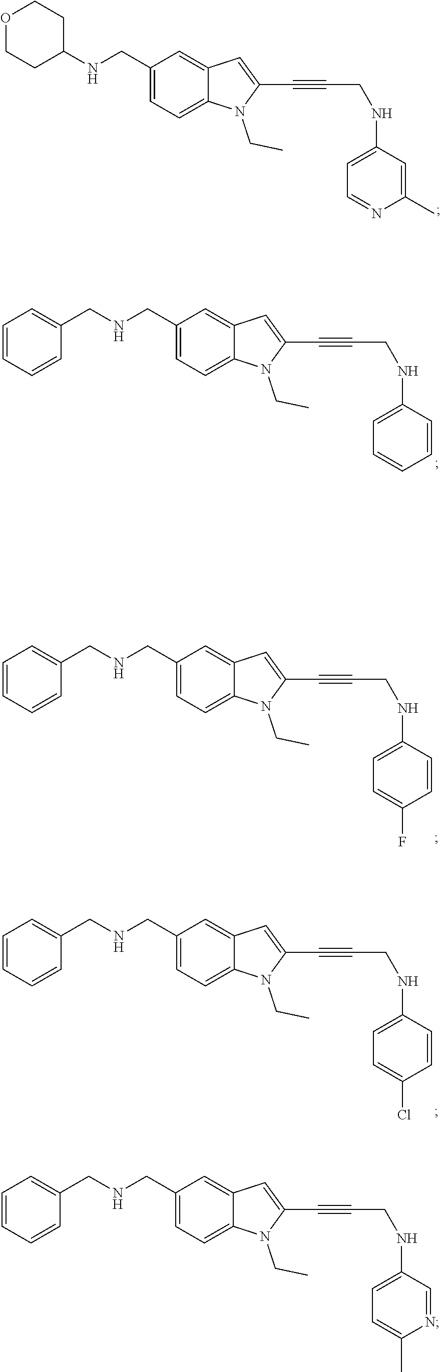 [SMILES: CCn1c(C#CCNc2ccnc(C)c2)cc2cc(CNC3CCOCC3)ccc21 ; CCn1c(C#CCNc2ccccc2)cc2cc(CNCc3ccccc3)ccc21 ; CCn1c(C#CCNc2ccc(F)cc2)cc2cc(CNCc3ccccc3)ccc21 ; CCn1c(C#CCNc2ccc(Cl)cc2)cc2cc(CNCc3ccccc3)ccc21 ; CCn1c(C#CCNc2ccc(C)nc2)cc2cc(CNCc3ccccc3)ccc21]

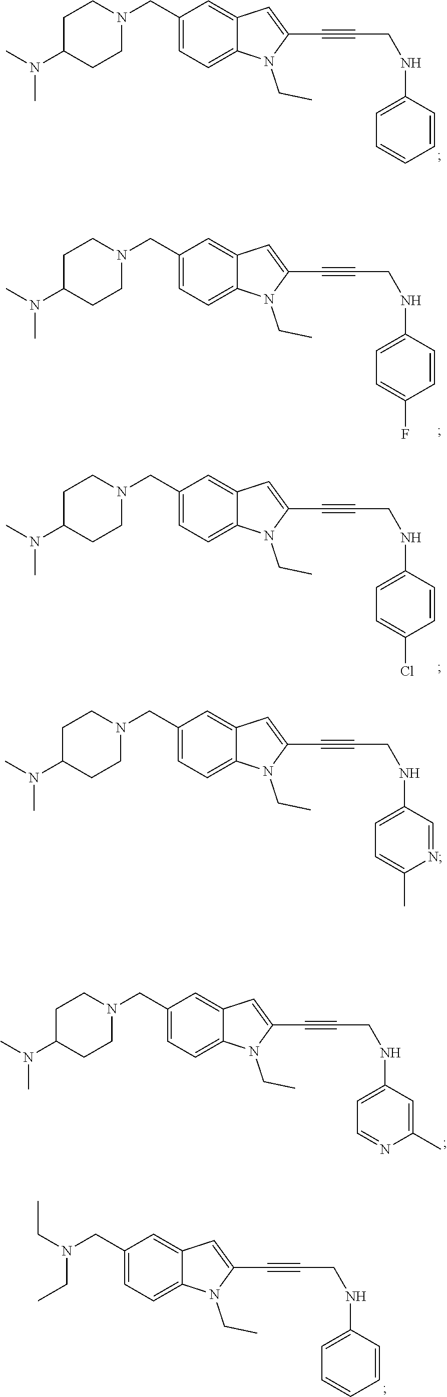

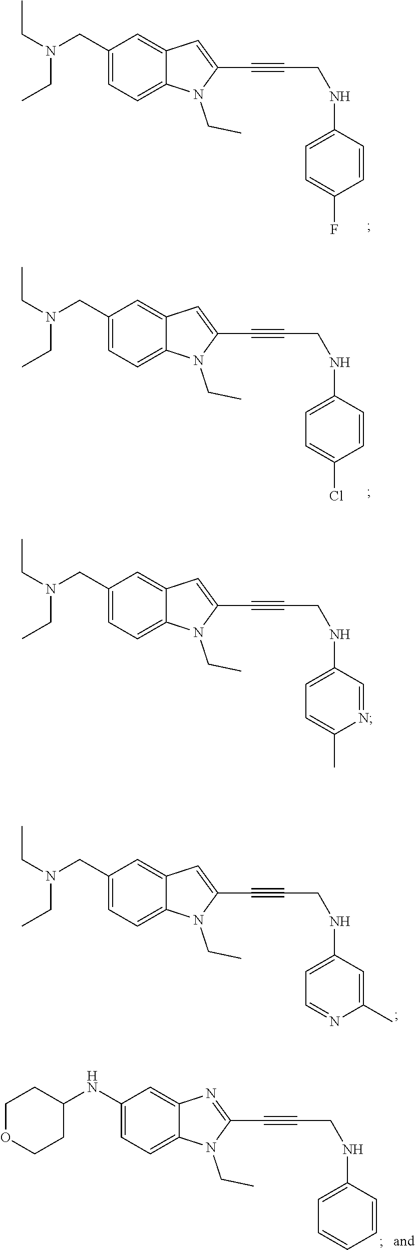 [SMILES: CCN(CC)Cc1ccc2c(c1)cc(C#CCNc1ccc(F)cc1)n2CC ; CCN(CC)Cc1ccc2c(c1)cc(C#CCNc1ccc(Cl)cc1)n2CC ; CCN(CC)Cc1ccc2c(c1)cc(C#CCNc1ccc(C)nc1)n2CC ; CCN(CC)Cc1ccc2c(c1)cc(C#CCNc1ccnc(C)c1)n2CC ; CCn1c(C#CCNc2ccccc2)nc2cc(NC3CCOCC3)ccc21]

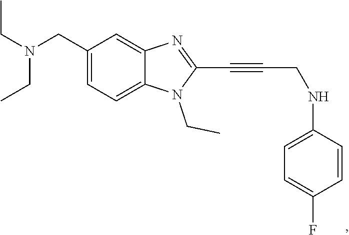 [SMILES: CCN(CC)Cc1ccc2c(c1)nc(C#CCNc1ccc(F)cc1)n2CC]

or a pharmaceutically-acceptable salt of any of the foregoing.

Non-limiting examples of compounds of the current disclosure include the following:

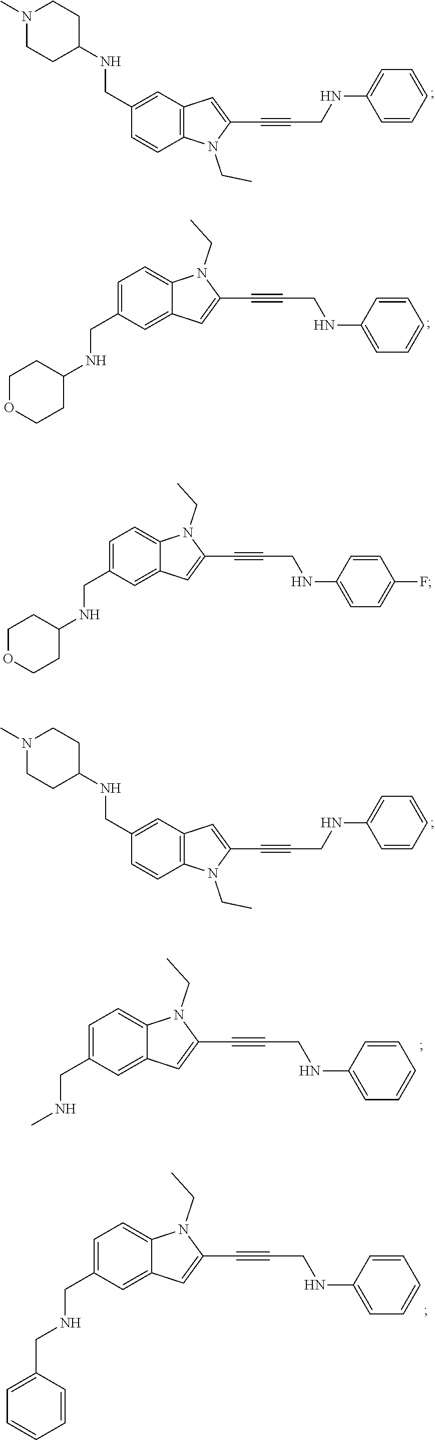 [SMILES: CCn1c(C#CCNc2ccccc2)cc2cc(CNC3CCN(C)CC3)ccc21 ; CCn1c(C#CCNc2ccccc2)cc2cc(CNC3CCOCC3)ccc21 ; CCn1c(C#CCNc2ccc(F)cc2)cc2cc(CNC3CCOCC3)ccc21 ; CCn1c(C#CCNc2ccccc2)cc2cc(CNC3CCN(C)CC3)ccc21 ; CCn1c(C#CCNc2ccccc2)cc2cc(CNC)ccc21 ; CCn1c(C#CCNc2ccccc2)cc2cc(CNCc3ccccc3)ccc21]

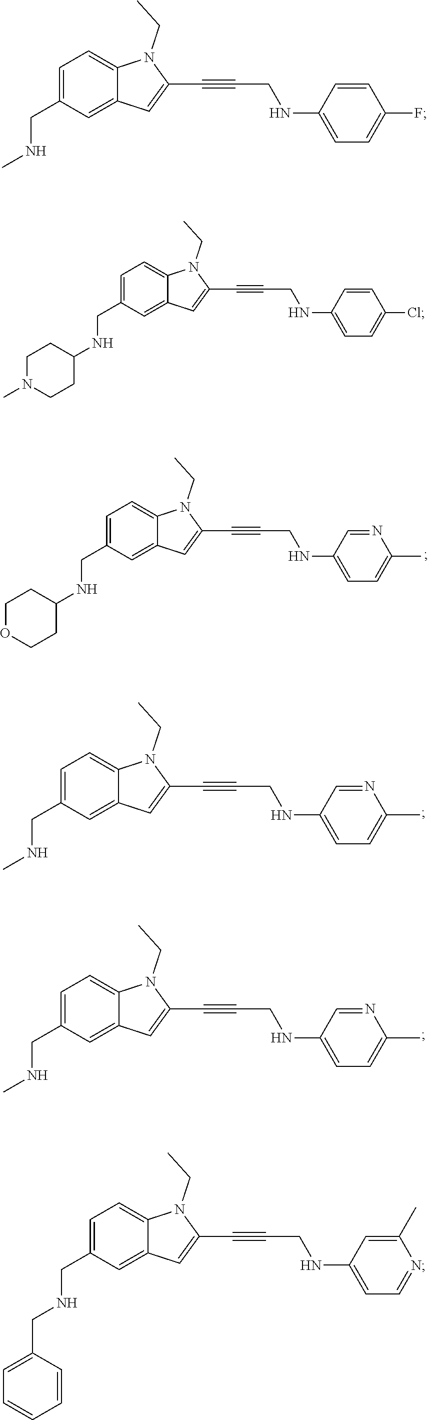

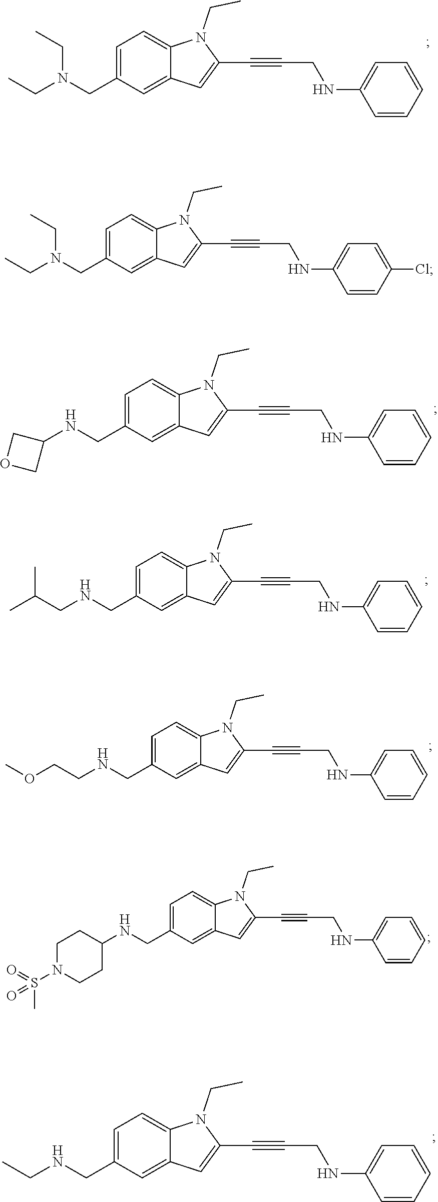

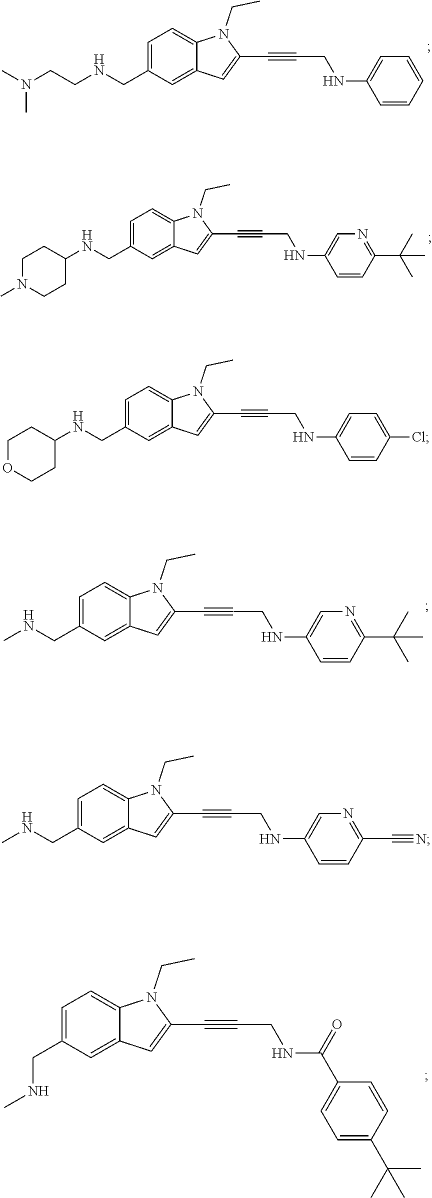

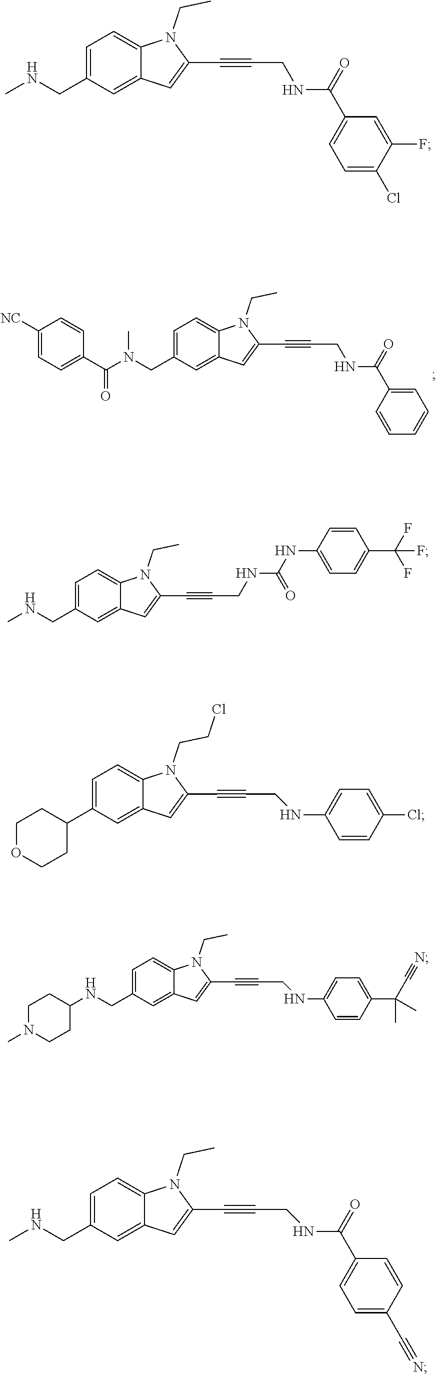 [SMILES: CCn1c(C#CCNC(=O)c2ccc(Cl)c(F)c2)cc2cc(CNC)ccc21 ; CCn1c(C#CCNC(=O)c2ccccc2)cc2cc(CN(C)C(=O)c3ccc(C#N)cc3)ccc21 ; CCn1c(C#CCNC(=O)Nc2ccc(C(F)(F)F)cc2)cc2cc(CNC)ccc21 ; ClCCn1c(C#CCNc2ccc(Cl)cc2)cc2cc(C3CCOCC3)ccc21 ; CCn1c(C#CCNc2ccc(C(C)(C)C#N)cc2)cc2cc(CNC3CCN(C)CC3)ccc21 ; CCn1c(C#CCNC(=O)c2ccc(C#N)cc2)cc2cc(CNC)ccc21]

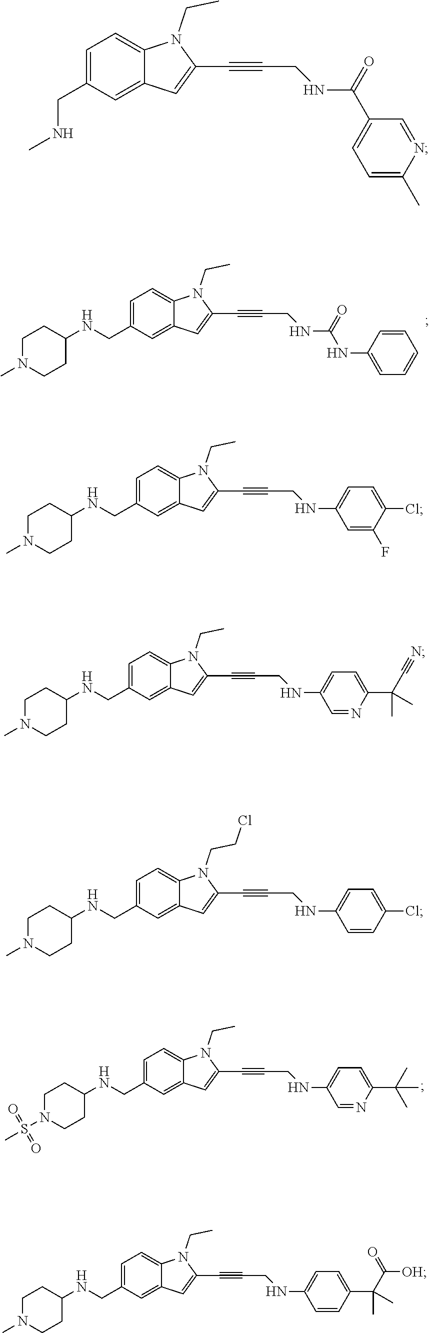

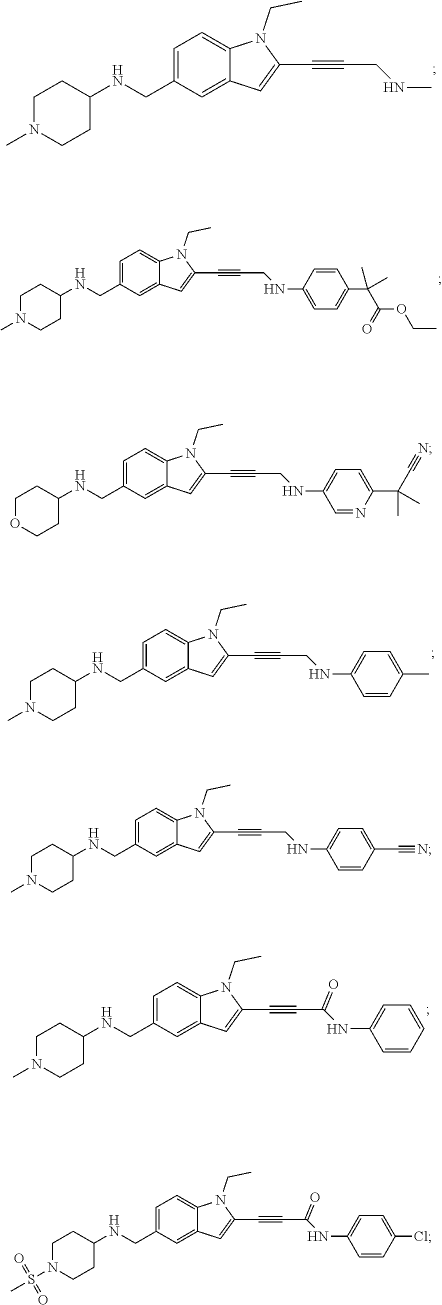

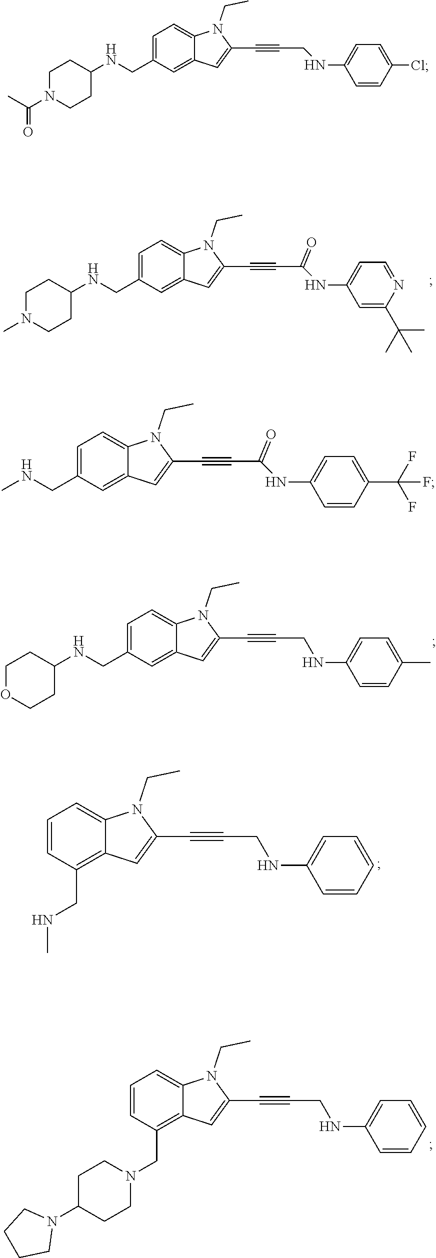

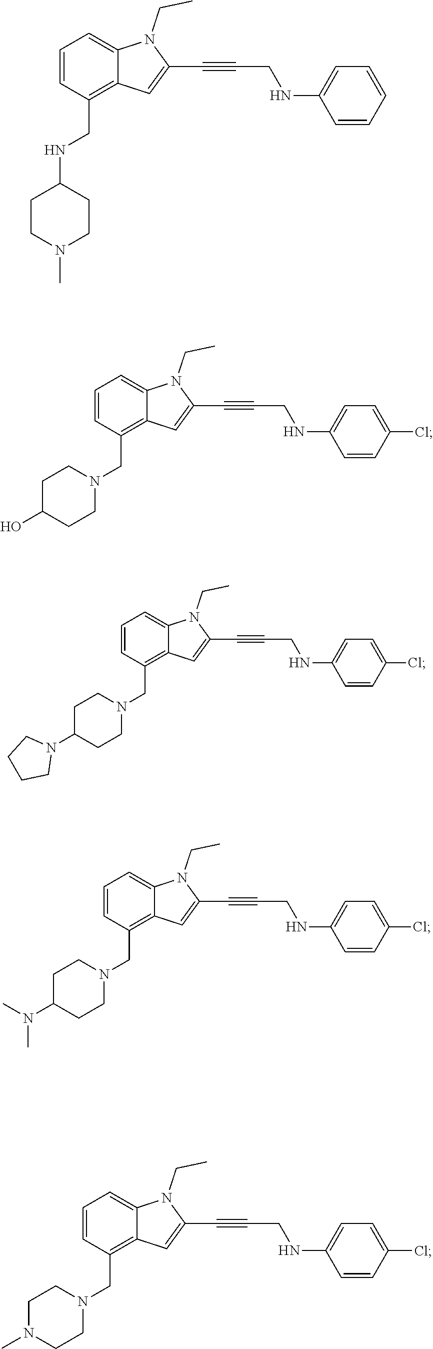 [SMILES: CCn1c(C#CCNc2ccccc2)cc2c(CNC3CCN(C)CC3)cccc21 ; CCn1c(C#CCNc2ccc(Cl)cc2)cc2c(CN3CCC(O)CC3)cccc21 ; CCn1c(C#CCNc2ccc(Cl)cc2)cc2c(CN3CCC(N4CCCC4)CC3)cccc21 ; CCn1c(C#CCNc2ccc(Cl)cc2)cc2c(CN3CCC(N(C)C)CC3)cccc21 ; CCn1c(C#CCNc2ccc(Cl)cc2)cc2c(CN3CCN(C)CC3)cccc21]

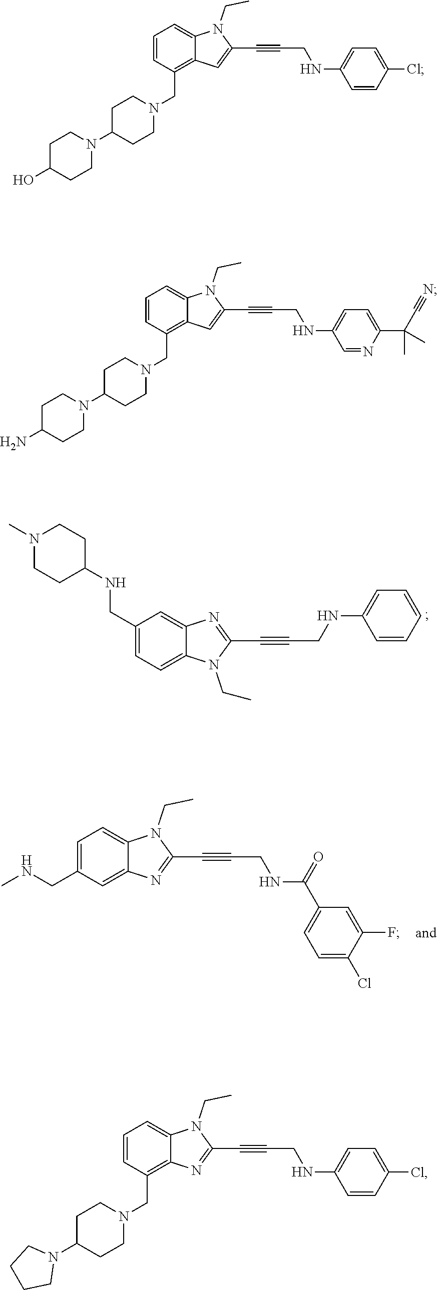 [SMILES: CCn1c(C#CCNc2ccc(Cl)cc2)cc2c(CN3CCC(N4CCC(O)CC4)CC3)cccc21 ; CCn1c(C#CCNc2ccc(C(C)(C)C#N)nc2)cc2c(CN3CCC(N4CCC(N)CC4)CC3)cccc21 ; CCn1c(C#CCNc2ccccc2)nc2cc(CNC3CCN(C)CC3)ccc21 ; CCn1c(C#CCNC(=O)c2ccc(Cl)c(F)c2)nc2cc(CNC)ccc21 ; "and" ; CCn1c(C#CCNc2ccc(Cl)cc2)nc2c(CN3CCC(N4CCCC4)CC3)cccc21]

or a pharmaceutically-acceptable salt of any of the foregoing.

Non-limiting examples of compounds of the current disclosure include the following:

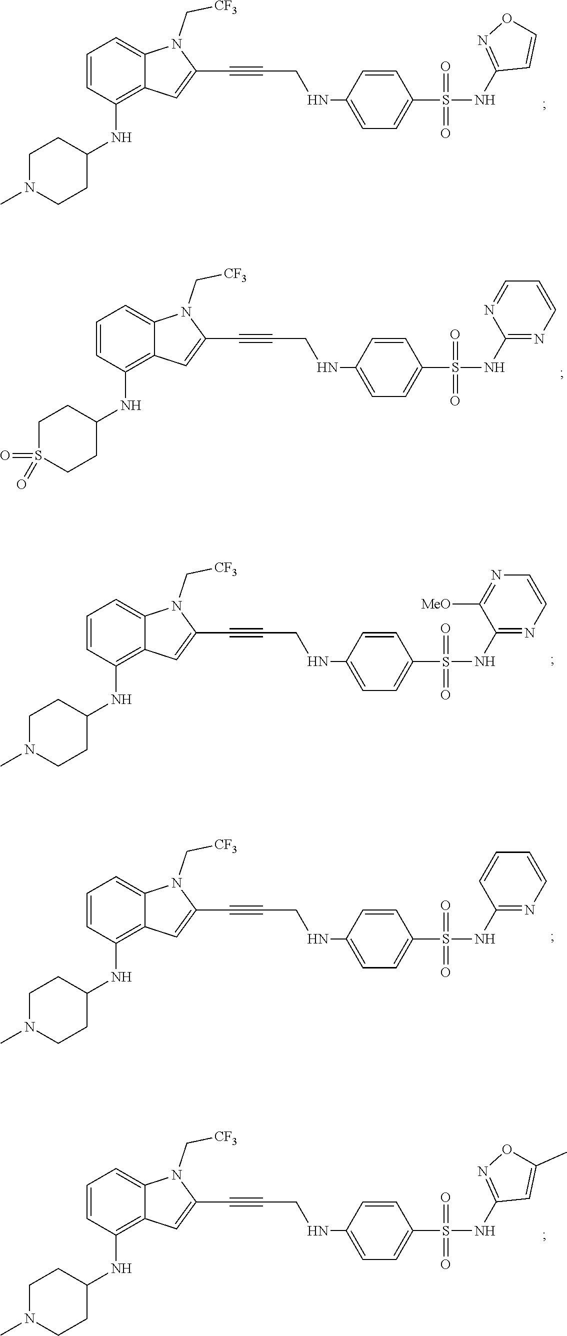

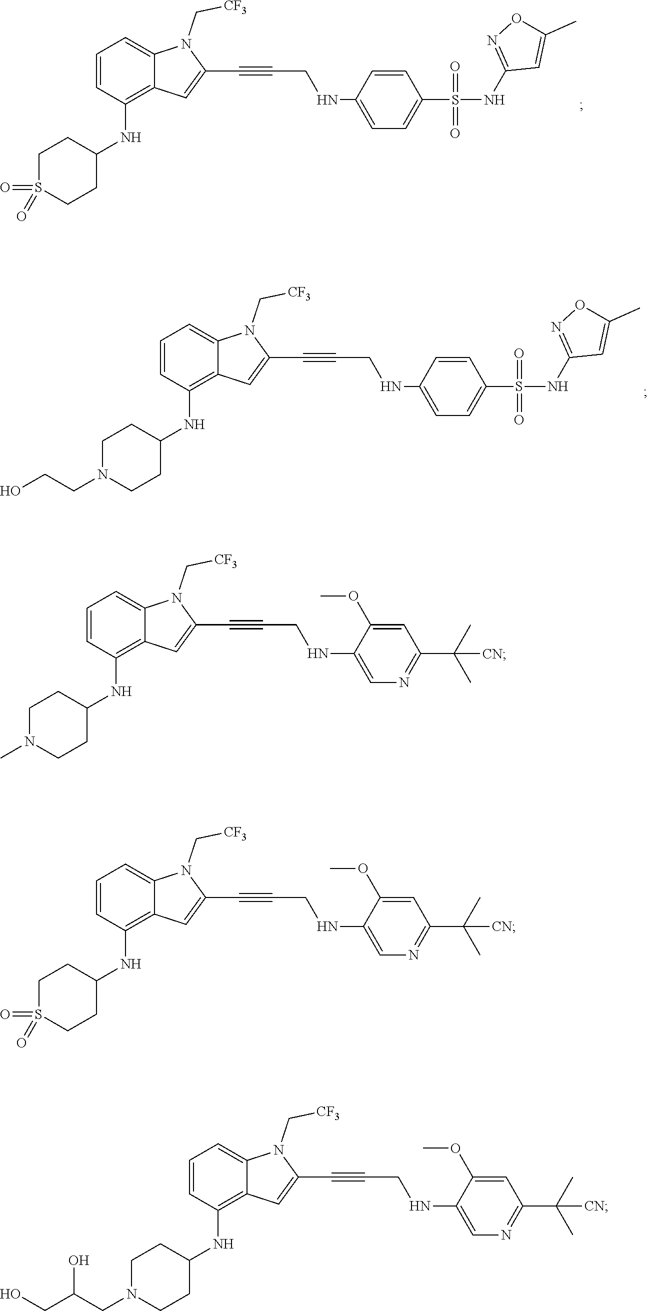

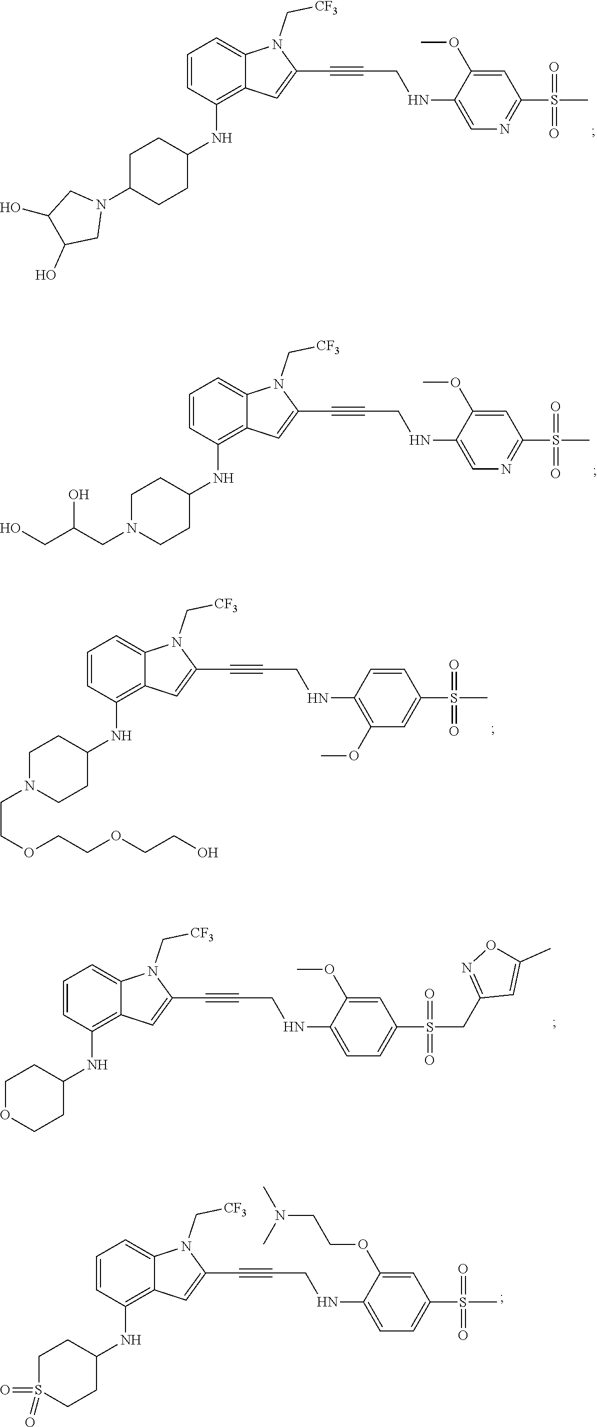 [SMILES: COc1cc(S(C)(=O)=O)ncc1NCC#Cc1cc2c(NC3CCC(N4CC(O)C(O)C4)CC3)cccc2n1CC(F)(F)F ; COc1cc(S(C)(=O)=O)ncc1NCC#Cc1cc2c(NC3CCN(CC(O)CO)CC3)cccc2n1CC(F)(F)F ; COc1cc(S(C)(=O)=O)ccc1NCC#Cc1cc2c(NC3CCN(CCOCCOCCO)CC3)cccc2n1CC(F)(F)F ; COc1cc(S(=O)(=O)Cc2cc(C)on2)ccc1NCC#Cc1cc2c(NC3CCOCC3)cccc2n1CC(F)(F)F ; CN(C)CCOc1cc(S(C)(=O)=O)ccc1NCC#Cc1cc2c(NC3CCS(=O)(=O)CC3)cccc2n1CC(F)(F)F]

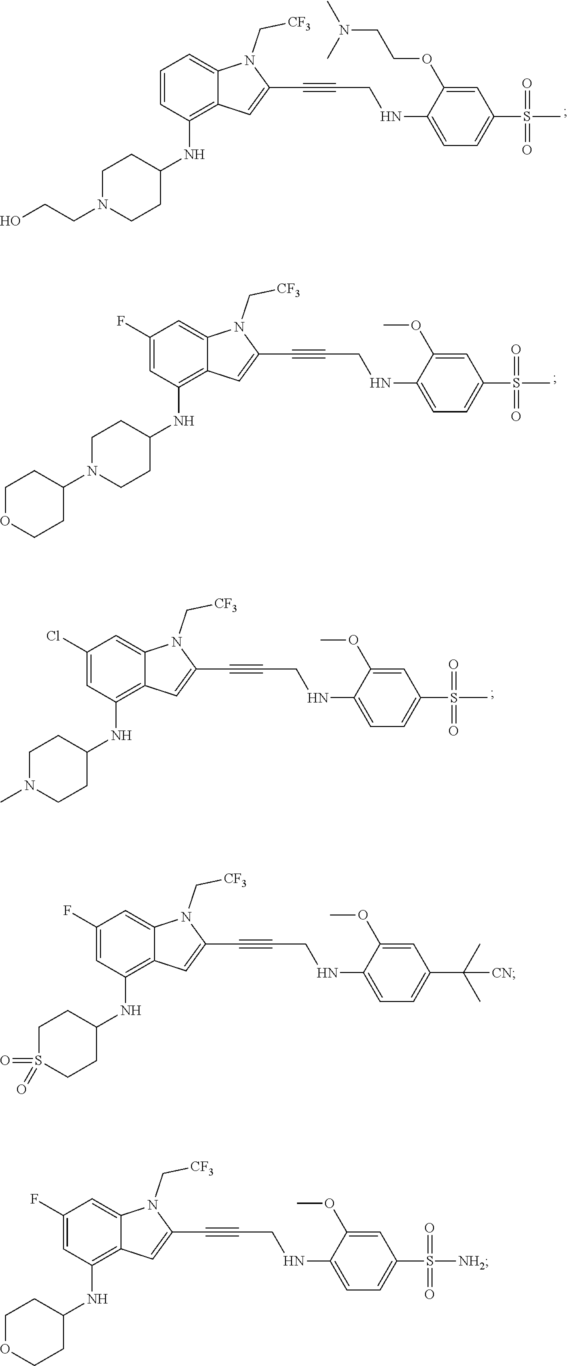

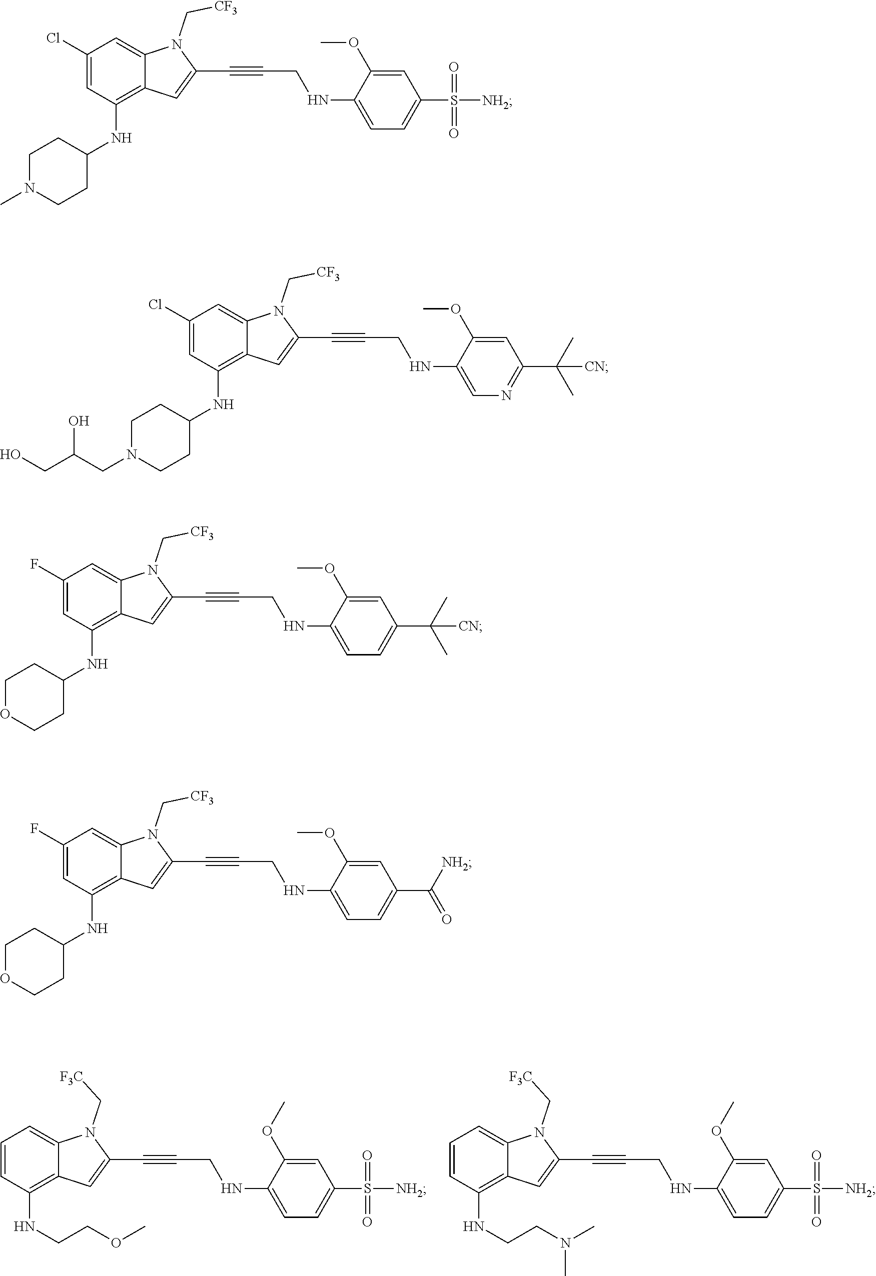 [SMILES: COc1cc(S(N)(=O)=O)ccc1NCC#Cc1cc2c(NC3CCN(C)CC3)cc(Cl)cc2n1CC(F)(F)F ; COc1cc(C(C)(C)C#N)ncc1NCC#Cc1cc2c(NC3CCN(CC(O)CO)CC3)cc(Cl)cc2n1CC(F)(F)F ; COc1cc(C(C)(C)C#N)ccc1NCC#Cc1cc2c(NC3CCOCC3)cc(F)cc2n1CC(F)(F)F ; COc1cc(C(N)=O)ccc1NCC#Cc1cc2c(NC3CCOCC3)cc(F)cc2n1CC(F)(F)F ; COCCNc1cccc2c1cc(C#CCNc1ccc(S(N)(=O)=O)cc1OC)n2CC(F)(F)F ; COc1cc(S(N)(=O)=O)ccc1NCC#Cc1cc2c(NCCN(C)C)cccc2n1CC(F)(F)F]

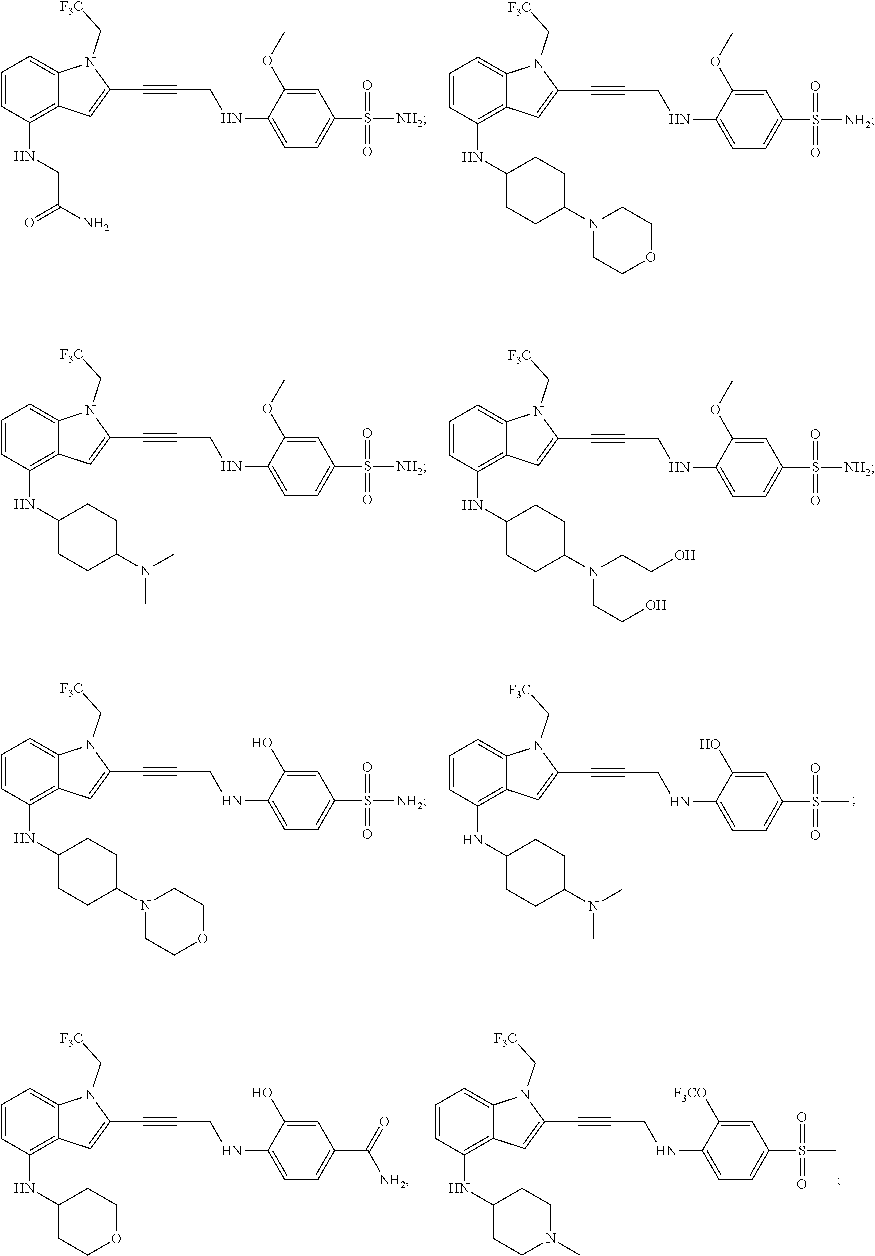

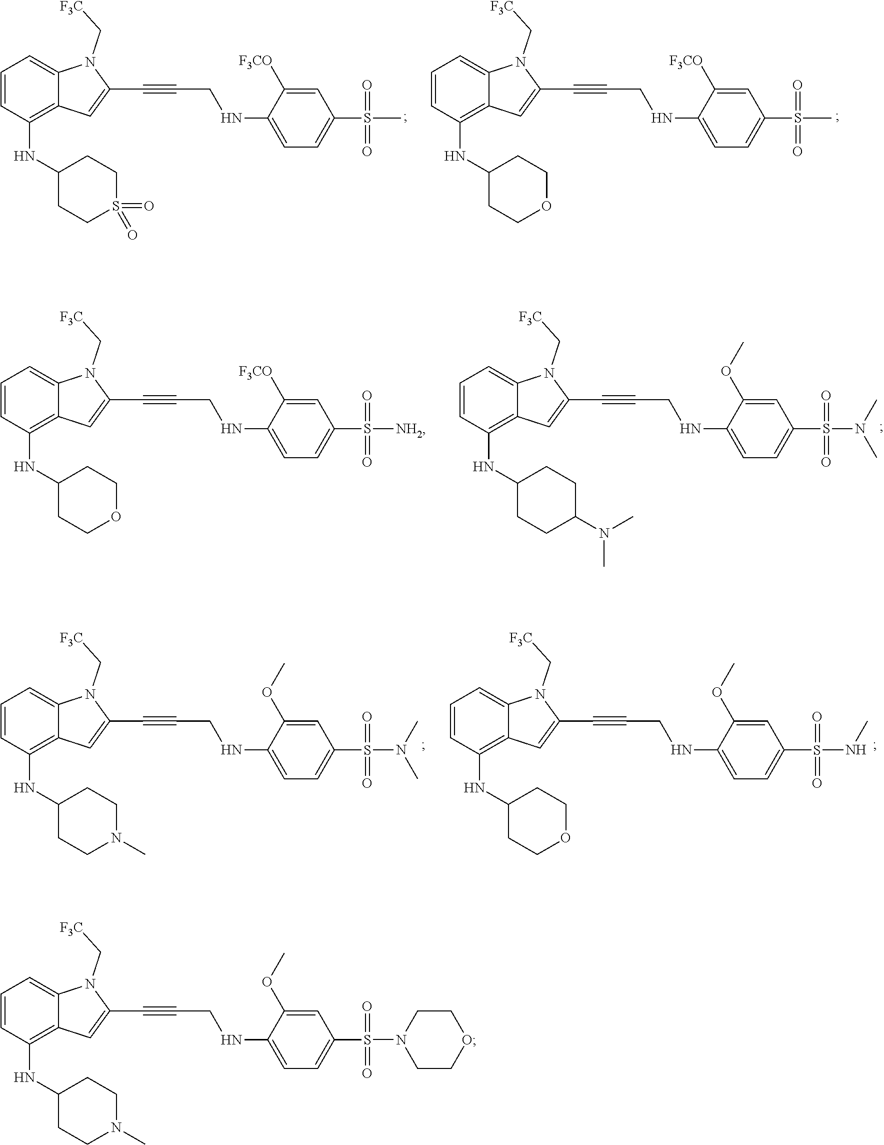

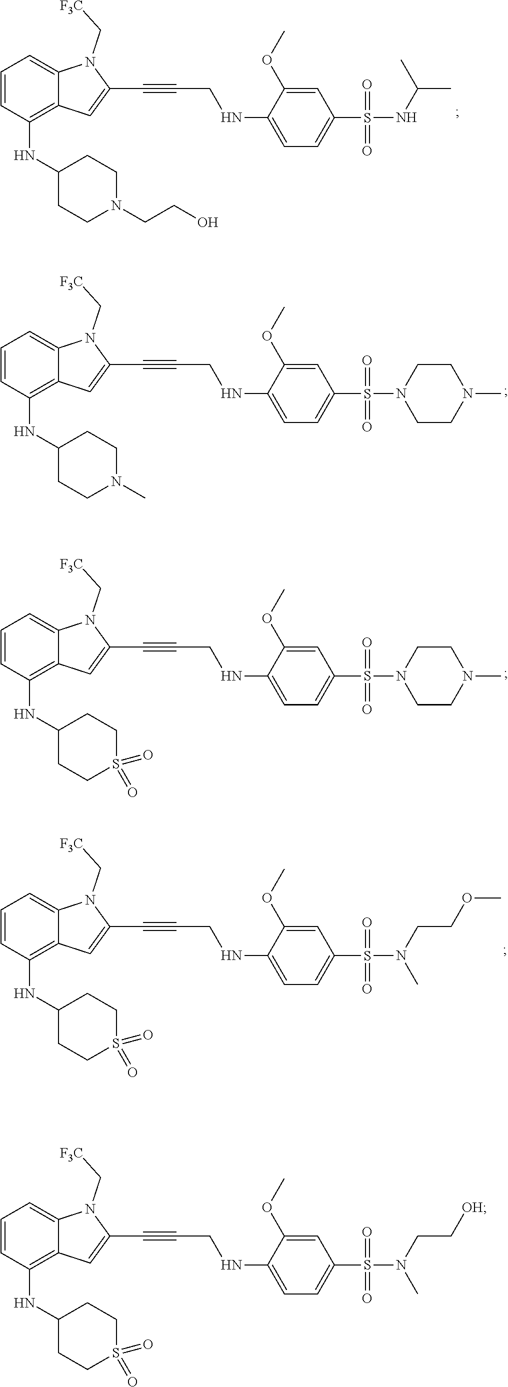 [SMILES: COc1cc(S(=O)(=O)NC(C)C)ccc1NCC#Cc1cc2c(NC3CCN(CCO)CC3)cccc2n1CC(F)(F)F ; COc1cc(S(=O)(=O)N2CCN(C)CC2)ccc1NCC#Cc1cc2c(NC3CCN(C)CC3)cccc2n1CC(F)(F)F ; COc1cc(S(=O)(=O)N2CCN(C)CC2)ccc1NCC#Cc1cc2c(NC3CCS(=O)(=O)CC3)cccc2n1CC(F)(F)F ; COCCN(C)S(=O)(=O)c1ccc(NCC#Cc2cc3c(NC4CCS(=O)(=O)CC4)cccc3n2CC(F)(F)F)c(OC)c1 ; COc1cc(S(=O)(=O)N(C)CCO)ccc1NCC#Cc1cc2c(NC3CCS(=O)(=O)CC3)cccc2n1CC(F)(F)F]

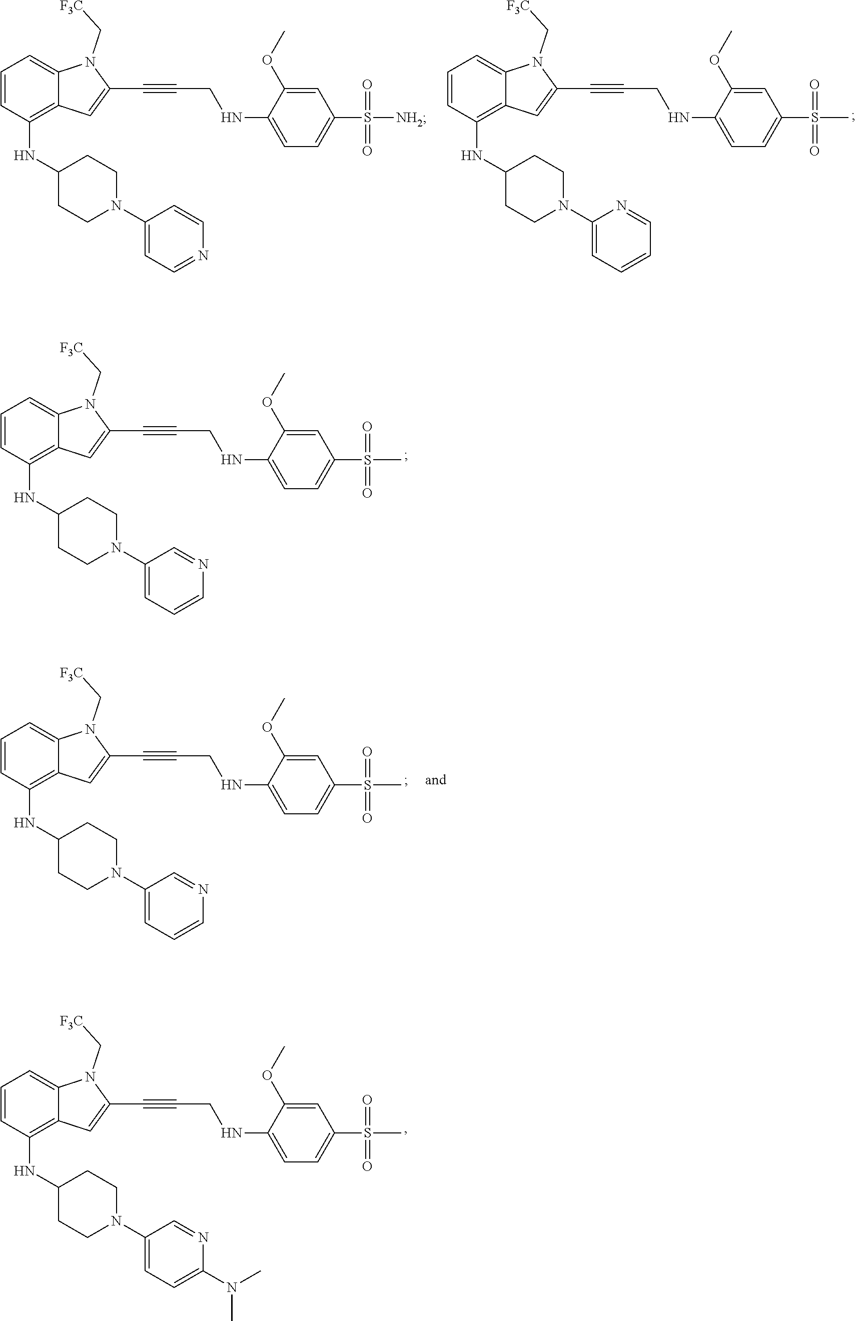 [SMILES: COc1cc(S(N)(=O)=O)ccc1NCC#Cc1cc2c(NC3CCN(c4ccncc4)CC3)cccc2n1CC(F)(F)F ; COc1cc(S(C)(=O)=O)ccc1NCC#Cc1cc2c(NC3CCN(c4ccccn4)CC3)cccc2n1CC(F)(F)F ; COc1cc(S(C)(=O)=O)ccc1NCC#Cc1cc2c(NC3CCN(c4cccnc4)CC3)cccc2n1CC(F)(F)F ; COc1cc(S(C)(=O)=O)ccc1NCC#Cc1cc2c(NC3CCN(c4cccnc4)CC3)cccc2n1CC(F)(F)F ; COc1cc(S(C)(=O)=O)ccc1NCC#Cc1cc2c(NC3CCN(c4ccc(N(C)C)nc4)CC3)cccc2n1CC(F)(F)F]

or a pharmaceutically-acceptable salt of any of the forgoing.

Non-limiting examples of compounds of the current disclosure include the following:

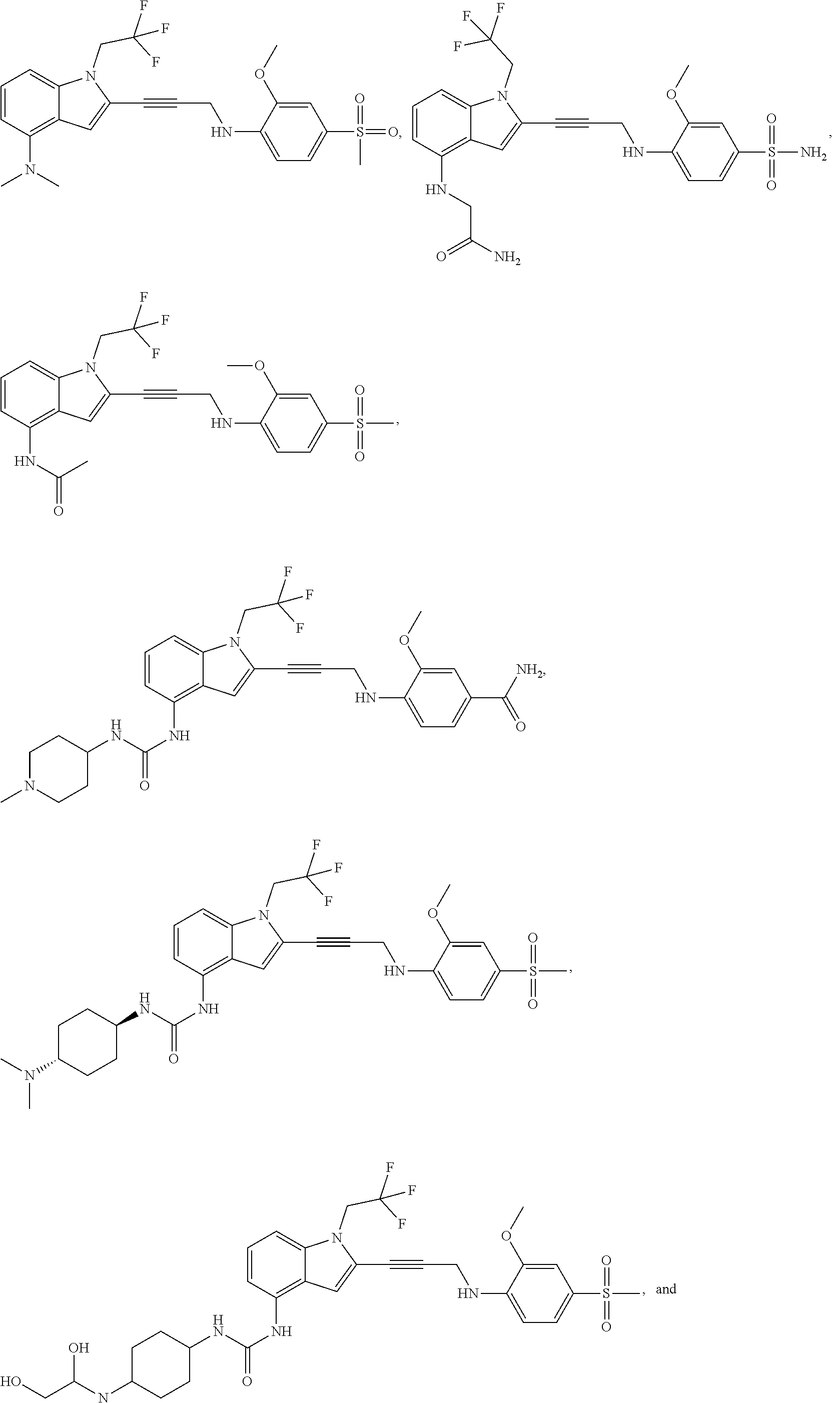

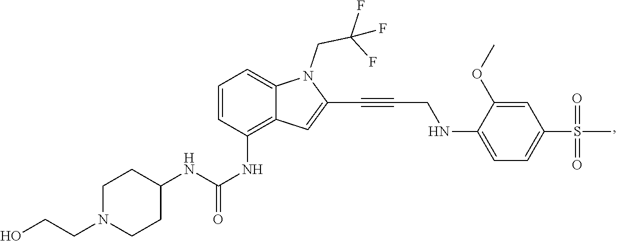 [SMILES: COc1cc(S(C)(=O)=O)ccc1NCC#Cc1cc2c(NC(=O)NC3CCN(CCO)CC3)cccc2n1CC(F)(F)F]

or a pharmaceutically-acceptable salt thereof.

Non-limiting examples of compounds of the current disclosure include the following:

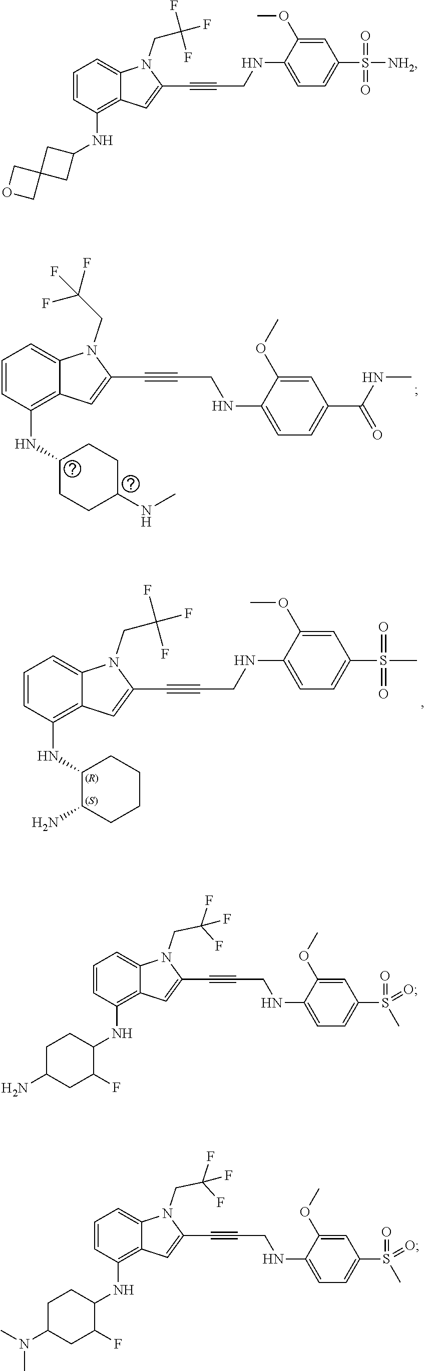

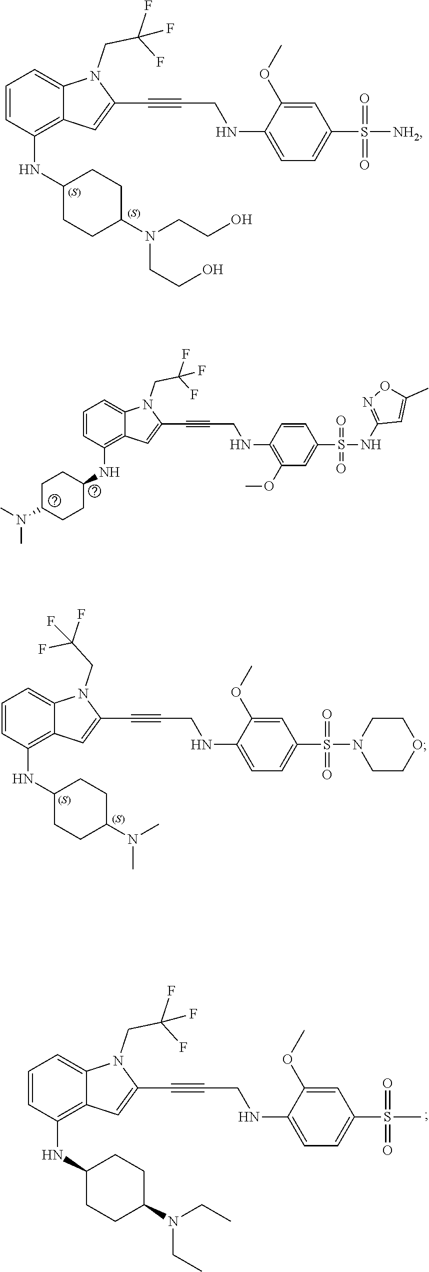

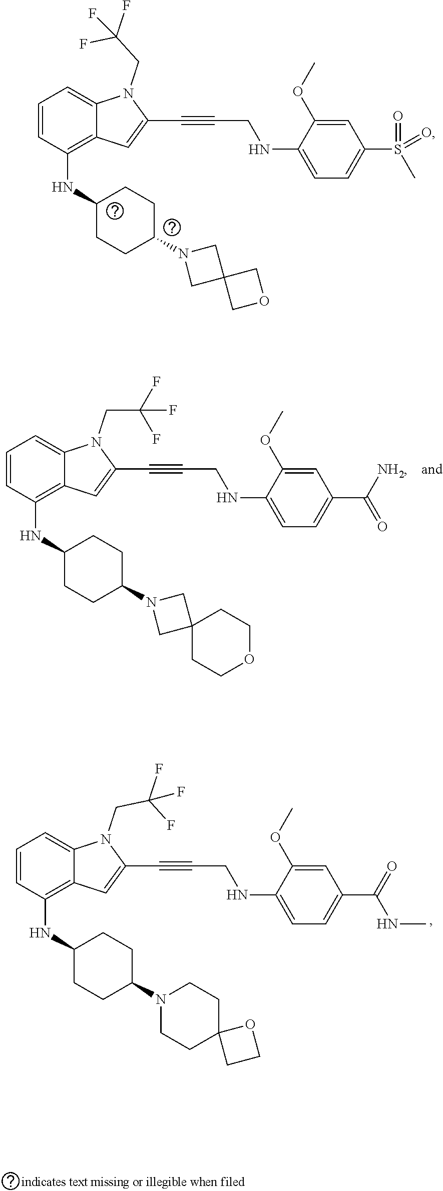 [SMILES: COc1cc(S(C)(=O)=O)ccc1NCC#Cc1cc2c(N[C@H]3CC[C@H](N4CC5(COC5)C4)CC3)cccc2n1CC(F)(F)F ; COc1cc(C(N)=O)ccc1NCC#Cc1cc2c(N[C@H]3CC[C@@H](N4CC5(CCOCC5)C4)CC3)cccc2n1CC(F)(F)F ; "and" ; CNC(=O)c1ccc(NCC#Cc2cc3c(N[C@H]4CC[C@@H](N5CCC6(CCO6)CC5)CC4)cccc3n2CC(F)(F)F)c(OC)c1]

or a pharmaceutically-acceptable salt thereof.

Non-limiting examples of compounds of the current disclosure include the following:

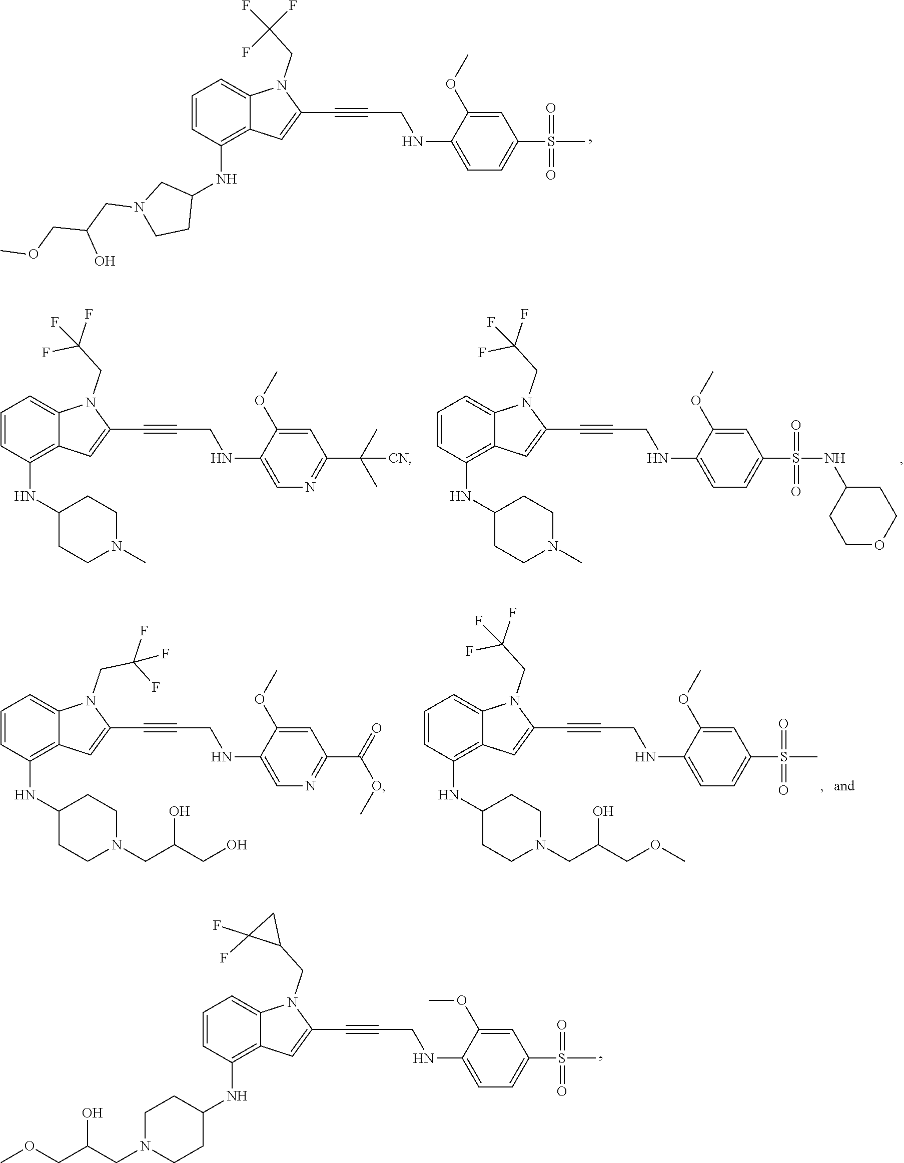

or a pharmaceutically-acceptable salt thereof.

Non-limiting examples of compounds of the current disclosure include the following:

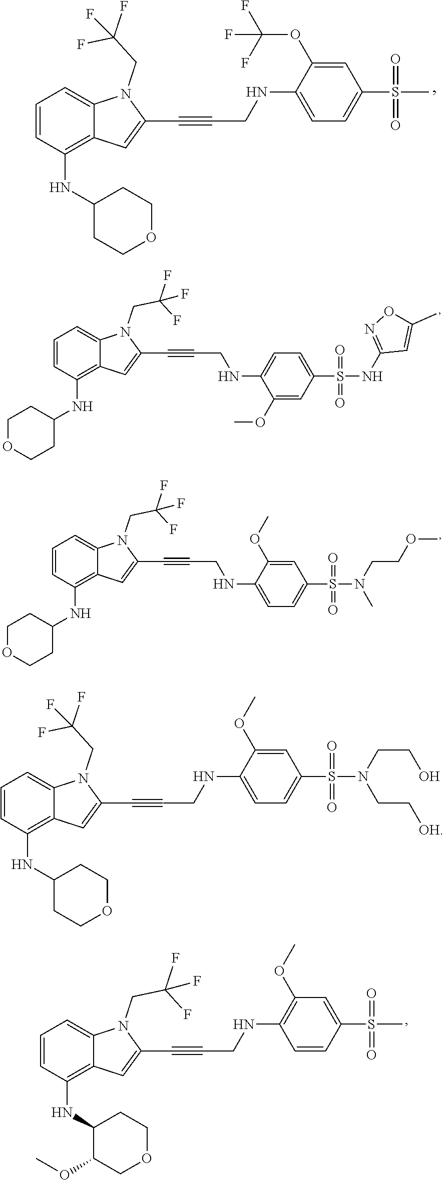

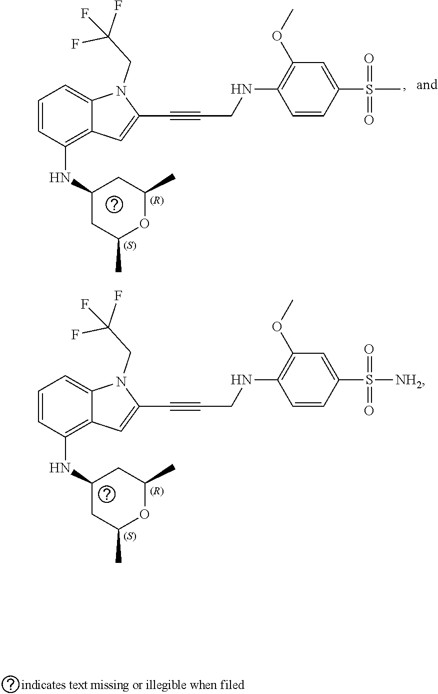

or a pharmaceutically-acceptable salt thereof.

Non-limiting examples of compounds of the current disclosure include the following:

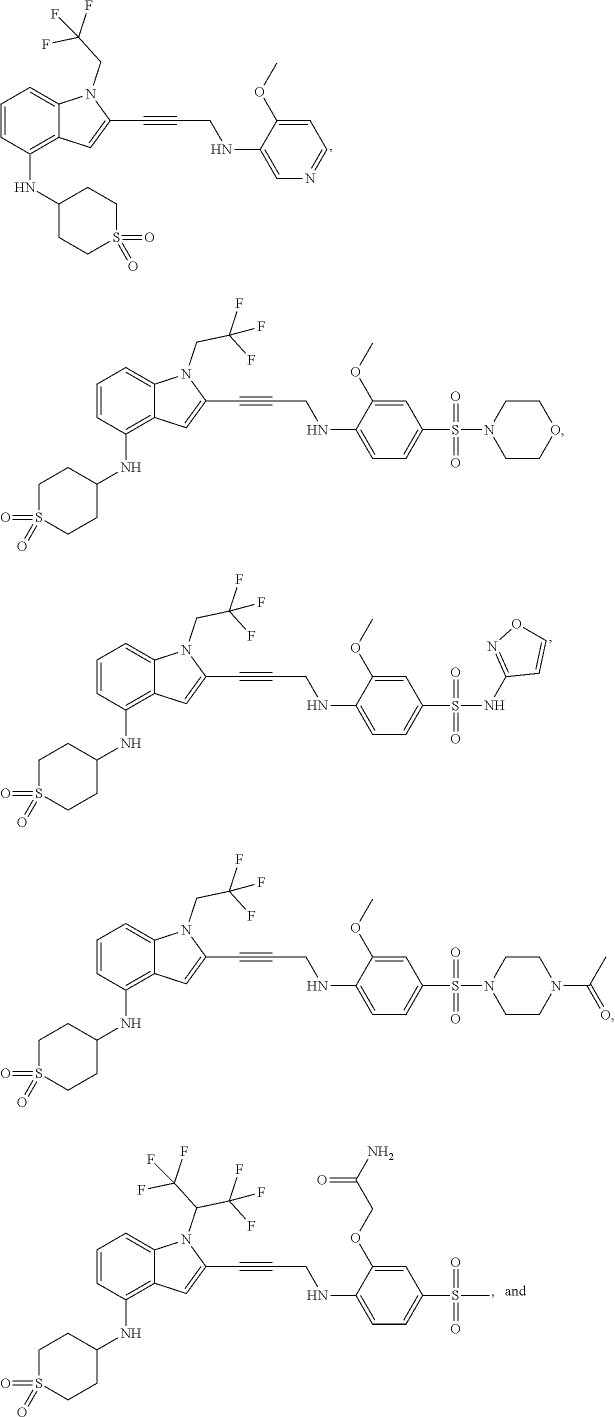 [SMILES: COc1ccncc1NCC#Cc1cc2c(NC3CCS(=O)(=O)CC3)cccc2n1CC(F)(F)F ; COc1cc(S(=O)(=O)N2CCOCC2)ccc1NCC#Cc1cc2c(NC3CCS(=O)(=O)CC3)cccc2n1CC(F)(F)F ; COc1cc(S(=O)(=O)Nc2ccon2)ccc1NCC#Cc1cc2c(NC3CCS(=O)(=O)CC3)cccc2n1CC(F)(F)F ; COc1cc(S(=O)(=O)N2CCN(C(C)=O)CC2)ccc1NCC#Cc1cc2c(NC3CCS(=O)(=O)CC3)cccc2n1CC(F)(F)F ; CS(=O)(=O)c1ccc(NCC#Cc2cc3c(NC4CCS(=O)(=O)CC4)cccc3n2C(C(F)(F)F)C(F)(F)F)c(OCC(N)=O)c1]

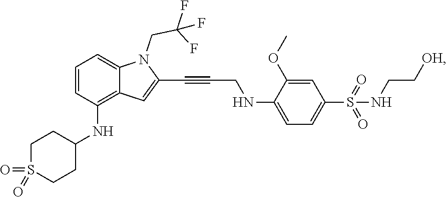 [SMILES: COc1cc(S(=O)(=O)NCCO)ccc1NCC#Cc1cc2c(NC3CCS(=O)(=O)CC3)cccc2n1CC(F)(F)F]

or a pharmaceutically-acceptable salt thereof.

Non-limiting examples of compounds of the current disclosure include the following:

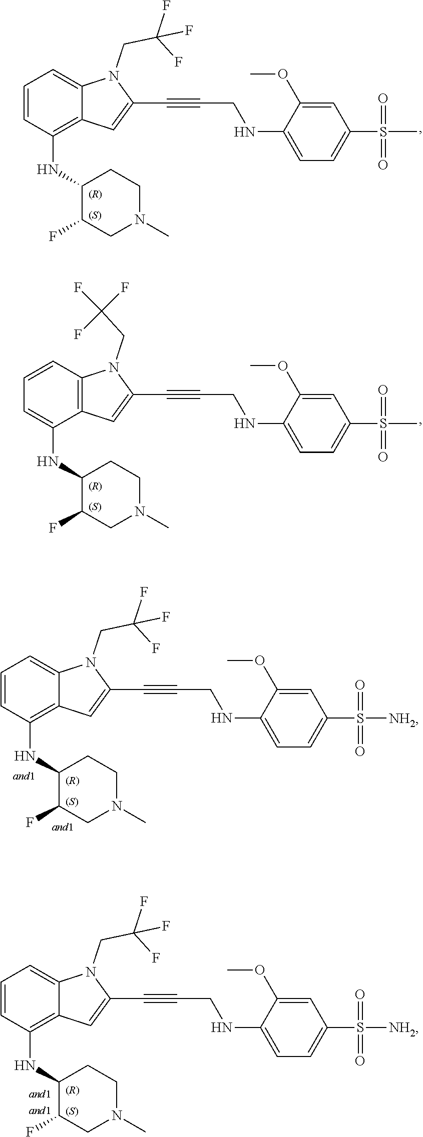

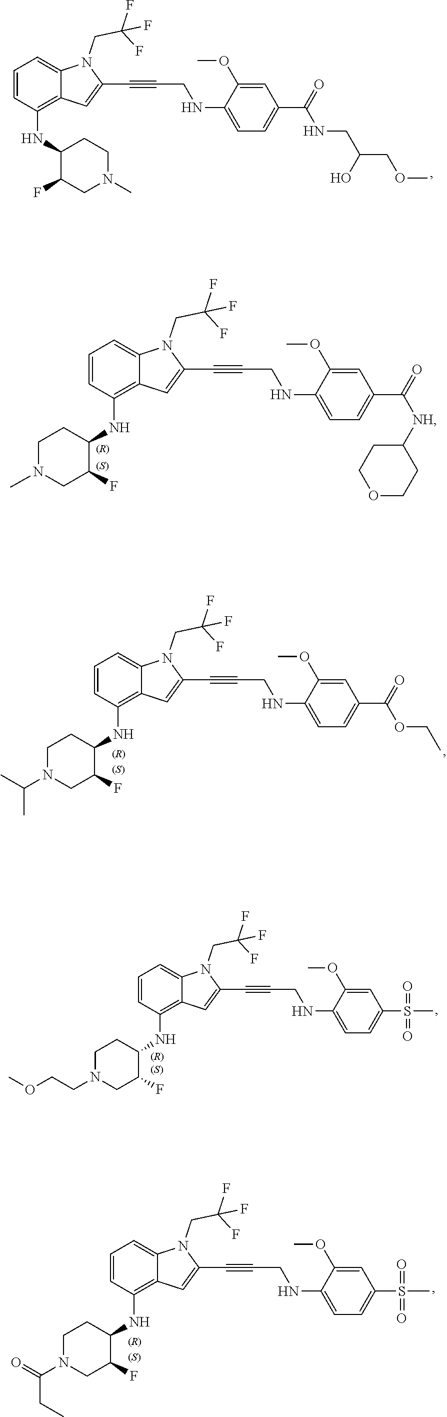

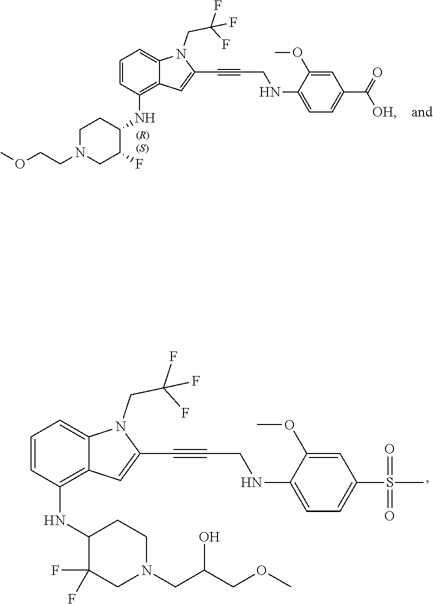

or a pharmaceutically-acceptable salt thereof.

Non-limiting examples of compounds of the current disclosure include the following:

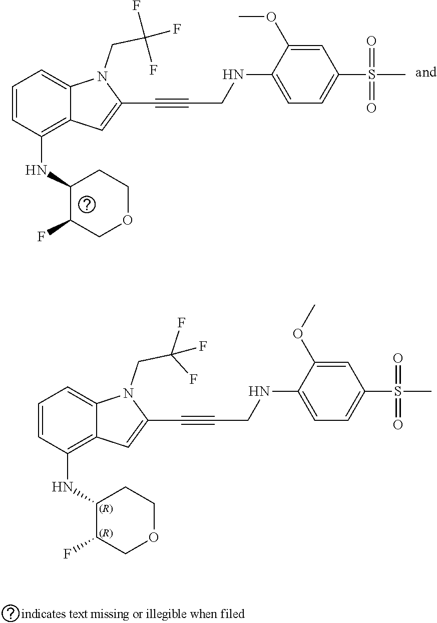

or a pharmaceutically-acceptable salt thereof.

In some embodiments, the disclosure provides a compound comprising: an indole group, wherein the indole group comprises: a) a haloalkyl group at a 1-position of the indole group; b) a first substituent at a 2-position of the indole group, wherein the first substituent is a cyclic group; and c) a second substituent, wherein the second substituent is substituted with at least halo-; or a pharmaceutically-acceptable salt thereof.

In some embodiments, the cyclic group is aryl, heteroaryl, or heterocyclyl, each of which is substituted or unsubstituted. In some embodiments, the cyclic group is unsubstituted aryl. In some embodiments, the cyclic group is substituted aryl. In some embodiments, the cyclic group is substituted phenyl. In some embodiments, the cyclic group is substituted or unsubstituted heteroaryl. In some embodiments, the heteroaryl is an aromatic 5-membered or 6-membered monocyclic ring. In some embodiments, the heteroaryl is thiazolyl, thiadiazolyl, pyrazolyl, thiophenyl, or oxadiazolyl. In some embodiments, the heteroaryl is pyridinyl or pyrimidinyl.

In some embodiments, the second substituent is at a 4-position of the indole group. In some embodiments, the second substituent is a second cyclic group that is substituted or unsubstituted. In some embodiments, the second cyclic group is heterocyclyl. In some embodiments, the heterocyclyl is piperidinyl. In some embodiments, the heterocyclyl is tetrahydropyranyl. In some embodiments, the heterocyclyl is substituted with fluoro-. In some embodiments, the heterocyclyl is substituted with chloro-. In some embodiments, the haloalkyl group is trifluoroethyl.

In some embodiments, the disclosure provides a compound, the compound comprising an indole group, wherein the indole group comprises: a) a substituted or unsubstituted non-cyclic group at a 3-position of the indole group; and b) a substituted or unsubstituted cyclic group at a 2-position of the indole group, wherein the compound increases a stability of a biologically-active conformation of a p53 mutant relative to a stability of a biologically-active conformation of the p53 mutant in an absence of the compound, or a pharmaceutically-acceptable salt thereof.

In some embodiments, the non-cyclic group is hydrogen. In some embodiments, the non-cyclic group is halo-. In some embodiments, the cyclic group is aryl, heteroaryl, heterocyclyl, or cycloalkylene, each of which is substituted or unsubstituted. In some embodiments, the cyclic group is aryl or heteroaryl, each of which is substituted or unsubstituted. In some embodiments, the cyclic group is substituted aryl. In some embodiments, the cyclic group is substituted phenyl. In some embodiments, the cyclic group is phenyl substituted with alkyl, cycloalkyl, alkoxy, an amine group, a carboxyl group, a carboxylic acid group, a carbamide group, or an amide group, each of which is substituted or unsubstituted; cyano, halo-, or hydrogen.

In some embodiments, the cyclic group is substituted heteroaryl. In some embodiments, the cyclic group is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, the cyclic group is pyridinyl, pyrimidinyl, thiadiazolyl, thiazolyl, pyrazolyl, thiophenyl, or oxadiazolyl, In some embodiments, the cyclic group is 1,3,5-thiadiazol-2-yl. In some embodiments, the cyclic group is 1,3,4-oxadiazol-2-yl or 1,2,4-oxadiazol-2-yl. In some embodiments, the cyclic group is pyridinyl.

In some embodiments, the indole group further comprises a substituent at a 4-position of the indole group. In some embodiments, the substituent is an amino group that is substituted or unsubstituted. In some embodiments, the amino group is substituted with a second cyclic group. In some embodiments, the second cyclic group is a heterocyclyl group substituted with at least halo-. In some embodiments, the heterocyclyl group is substituted with at least fluoro-. In some embodiments, the heterocyclyl group is substituted with at least chloro-. In some embodiments, the heterocyclyl group is piperidinyl. In some embodiments, the heterocyclyl group is tetrahydropyranyl.

Non-limiting examples of compounds of the disclosure include compounds of any of the following formulae:

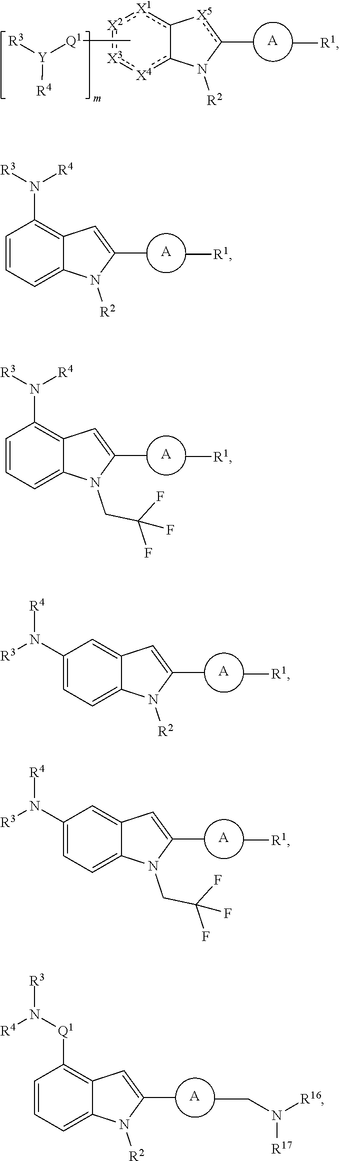

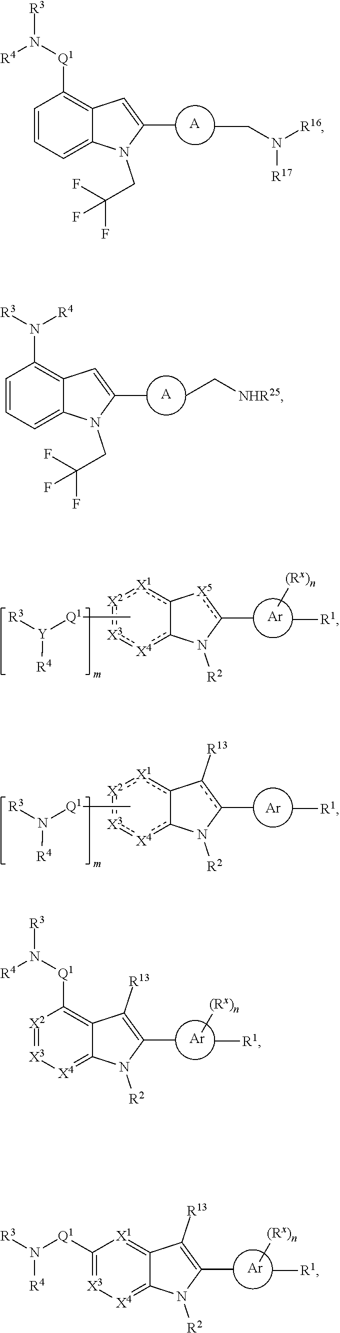

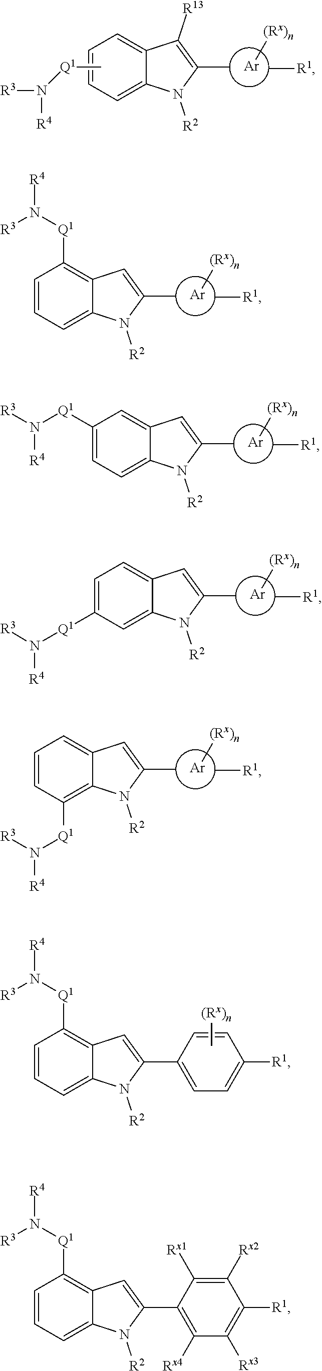

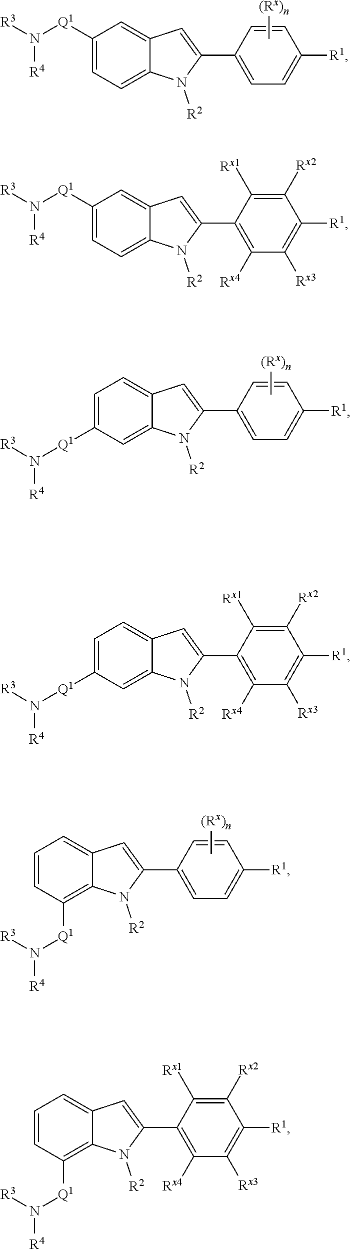

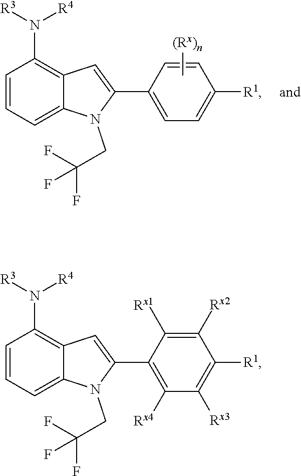

or a pharmaceutically-acceptable salt thereof.

In some embodiments, the disclosure provides a compound of the formula:

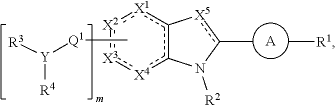

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a substituted or unsubstituted ring;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, A is substituted or unsubstituted aryl, heteroaryl, heterocyclyl, cycloalkylene. In some embodiments, A is a 6-carbon monocyclic or 10-carbon bicyclic aromatic ring system wherein 0, 1, 2, 3, or 4 atoms of each ring are optionally substituted. In some embodiments, A is naphthyl. In some embodiments, A is indazolyl.

In some embodiments, A is substituted aryl. In some embodiments, A is substituted phenyl. In some embodiments, A is phenyl substituted with alkyl, cycloalkyl, alkoxy, an amine group, a carboxyl group, a carboxylic acid group, a carbamide group, or an amide group, each of which is substituted or unsubstituted; cyano, halogen, or hydrogen. In some embodiments, A is phenyl substituted with alkyl, wherein alkyl is substituted. In some embodiments, A is phenyl substituted with alkyl, wherein alkyl is substituted with an amino group that is substituted or unsubstituted. In some embodiments, A is phenyl substituted with an amine group that is substituted or unsubstituted. In some embodiments, A is phenyl substituted with a carboxyl group that is substituted or unsubstituted. In some embodiments, A is phenyl substituted with cyano. In some embodiments, A is phenyl substituted with halo-.

In some embodiments, A is substituted or unsubstituted heterocyclyl. In some embodiments, A is substituted heterocyclyl.

In some embodiments, A is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, A is an aromatic 8-membered, 9-membered, 10-membered, 11-membered, or 12-membered bicyclic ring system comprising 1, 2, 3, 4, 5, or 6 heteroatoms, wherein each heteroatom is independently selected from O, N, or S. In some embodiments, A is an aromatic 5-membered, 6-membered, 7-membered, or 8-membered monocyclic ring system comprising 1, 2, or 3 heteroatoms, and the aromatic 5-membered, 6-membered, 7-membered, or 8-membered monocyclic ring system is substituted. In some embodiments, A is an 8-membered, 9-membered, 10-membered, 11-membered, or 12-membered bicyclic ring system having 1, 2, 3, 4, 5, or 6 heteroatoms, and the 8-membered, 9-membered, 10-membered, 11-membered, or 12-membered bicyclic ring system is substituted.

In some embodiments, A is pyridinyl, pyrimidinyl, thiadiazolyl, thiazolyl, pyrazolyl, thiophenyl, or oxadiazolyl, each of which is independently substituted or unsubstituted. In some embodiments, A is 1,3,5-thiadiazol-2-yl. In some embodiments, A is 1,3,4-oxadiazol-2-yl or 1,2,4-oxadiazol-2-yl. In some embodiments, A is 1,3,4-oxadiazol-2-yl.

In some embodiments, m is 1. In some embodiments, m is 2. In some embodiments, Q¹ is alkylene, alkenylene, or alkynylene, each of which is independently substituted or unsubstituted, or a bond. In some embodiments, Q¹ is a bond. In some embodiments, Y is N.

In some embodiments, R² is hydrogen. In some embodiments, R² is substituted or unsubstituted alkyl. In some embodiments, R² is trifluoroethyl. In some embodiments, R² is cycloalkyl.

In some embodiments, R¹ is —C(O)R¹⁶, —C(O)OR¹⁶, —C(O)NR¹⁶R¹⁷, —OR¹⁶, —NR¹⁶R¹⁷, —NR¹⁶C(O)R¹⁶, —OC(O)R¹⁶, alkyl, alkoxy, aryl, heteroaryl, or heterocyclyl, each of which is independently substituted or unsubstituted; cyano, halo, or halogen. In some embodiments, R¹ is —NR¹⁶R¹⁷. In some embodiments, R¹ is substituted alkyl.

In some embodiments, each R³ and R⁴ is independently aryl, heteroaryl, or heterocyclyl, each of which is independently substituted or unsubstituted; or hydrogen. In some embodiments, R³ is hydrogen, and R⁴ is heterocyclyl substituted at least with halo-. In some embodiments, R⁴ is heterocyclyl substituted with fluoro. In some embodiments, R⁴ is heterocyclyl substituted with chloro.

In some embodiments, R¹³ is alkyl, alkenyl, hydrogen, or halogen. In some embodiments, R¹³ is hydrogen.

In some embodiments, the compound has the formula:

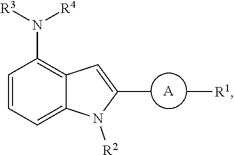

or a pharmaceutically-acceptable salt thereof, wherein the variables are as defined above.

In some embodiments, the compound has the formula:

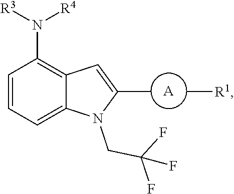

or a pharmaceutically-acceptable salt thereof, wherein the variables are as defined above.

In some embodiments, the compound has the formula:

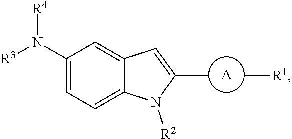

or a pharmaceutically-acceptable salt thereof, wherein the variables are as defined above.

In some embodiments, the compound has the formula:

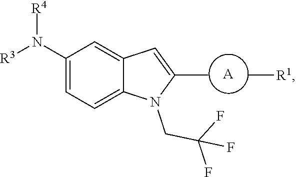

or a pharmaceutically-acceptable salt thereof, wherein the variables are as defined above.

In some embodiments, the disclosure provides a compound of the formula:

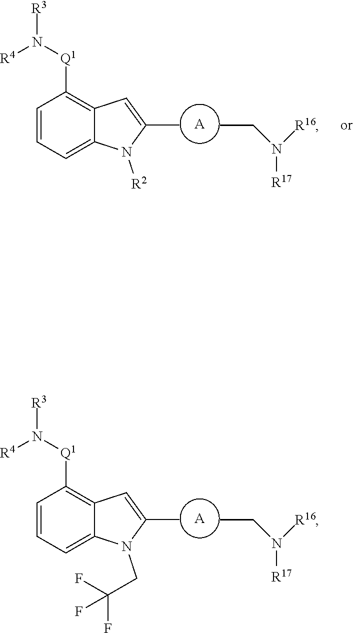

or a pharmaceutically-acceptable salt thereof, wherein the variables are as defined above.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

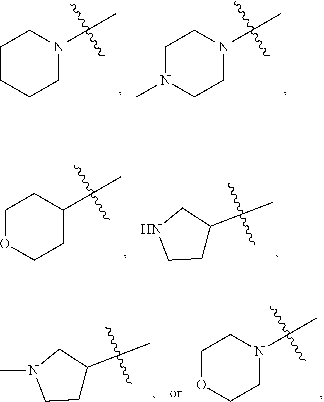

wherein the ring is substituted or unsubstituted. In some embodiments, R is H, and R⁴ is a ring that is

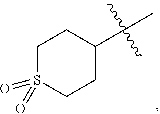 [SMILES: *C1CCS(=O)(=O)CC1]

wherein the ring is substituted or unsubstituted. In some embodiments, R³ is H, and R⁴ is a ring that is

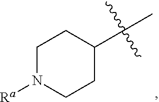

wherein the ring is substituted or unsubstituted. In some embodiments, R^{a} is alkylene. In some embodiments, R^{a} is methyl. In some embodiments, R³ is H, and R⁴ is a ring that is

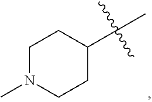 [SMILES: *C1CCN(C)CC1]

wherein the ring is substituted or unsubstituted. In some embodiments, R³ is H, and R⁴ is a ring that is

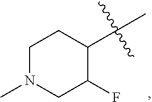 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

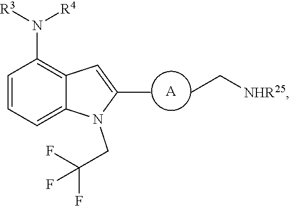

wherein R²⁵ is —C(O)R¹⁶, —C(O)NR¹⁶R¹⁷, alkyl, alkenyl, alkynyl, aryl, heteroaryl, or heterocyclyl, each of which is independently substituted or unsubstituted, or hydrogen. In some embodiments, R²⁵ is aryl that is substituted or unsubstituted. In some embodiments, R²⁵ is substituted phenyl. In some embodiments, R²⁵ is —C(O)R¹⁶, wherein R¹⁶ is alkyl, aryl, heteroaryl, or heterocyclyl. In some embodiments, R²⁵ is —C(O)R¹⁶, wherein R¹⁶ is substituted phenyl.

In some embodiments, the disclosure provides a compound of the formula:

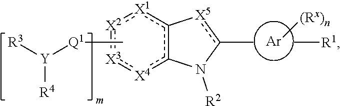

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Ar is unsubstituted or substituted aryl;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
n is 0, 1, 2, 3, or 4;
Y is N, O, or absent;
each R^{x} and R¹ is independently C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted; cyano, halo, or hydrogen; or R¹ and R^{x} together with Ar form a fused ring;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

The pattern of dashed bonds can be chosen to provide an aromatic system, for example, an indole, an indolene, a pyrrolopyridine, a pyrrolopyrimidine, or a pyrrolopyrazine. In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Ar is a 6-carbon monocyclic or 10-carbon bicyclic aromatic ring system wherein 0, 1, 2, 3, or 4 atoms of each ring are optionally substituted. In some embodiments, Ar is phenyl. In some embodiments, Ar is naphthyl. In some embodiments, Ar is indazolyl.

R¹ can be —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen. In some embodiments, R¹ is methyl, cyclohexyl, methylene, methoxy, or benzyl. In some embodiments, R¹ is fluoro or chloro. In some embodiments, R¹ is phenyl. In some embodiments, R¹ is hydrogen.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is alkyl substituted with —C(O)NR¹⁶R¹⁷. In some embodiments, R¹ is methyl substituted with —C(O)NR¹⁶R¹⁷. In some embodiments, R¹ is alkyl substituted with —C(O)OR¹⁶. In some embodiments, R¹ is methyl substituted with COOH.

In some embodiments, m is 1, 2, 3, or 4. In some embodiments, m is 1. In some embodiments, X³ is carbon atom connected to Q¹, and m is 1. In some embodiments, n is 1, 2, or 3. In some embodiments, n is 1. In some embodiments, n is 2. In some embodiments, n is 0.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

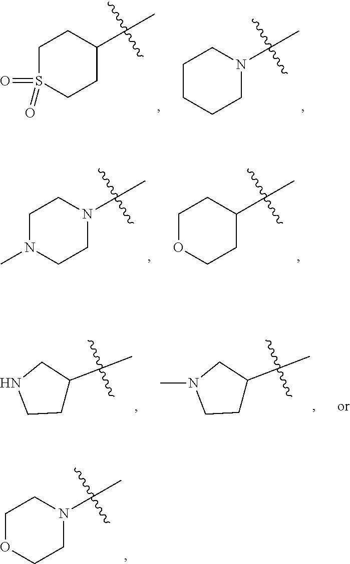

wherein the ring is substituted or unsubstituted. In some embodiments, R³ is H, and R⁴ is a ring that is

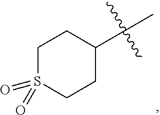 [SMILES: *C1CCS(=O)(=O)CC1]

wherein the ring is substituted or unsubstituted. In some embodiments, R³ is H, and R⁴ is a ring that is

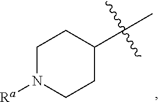

wherein the ring is substituted or unsubstituted. In some embodiments, R^{a} is alkylene. In some embodiments, R^{a} is methyl. In some embodiments, R³ is H, and R⁴ is a ring that is

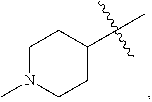 [SMILES: *C1CCN(C)CC1]

wherein the ring is substituted or unsubstituted. In some embodiments, R³ is H, and R⁴ is a ring that is

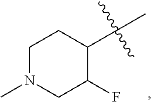 [SMILES: *C1CCN(C)CC1F]

wherein the ring is substituted or unsubstituted. In some embodiments, R³ is H, and R⁴ is a ring that is

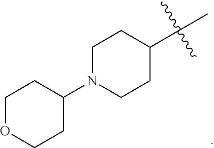 [SMILES: *C1CCN(C2CCOCC2)CC1]

In some embodiments, the disclosure provides a compound of the formula:

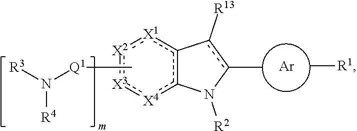

wherein the variables are as defined above.

In some embodiments, the disclosure provides a compound of the formula:

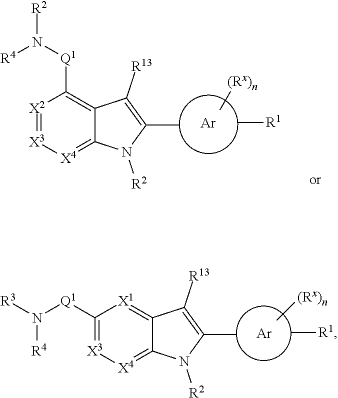

wherein:
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
Ar is unsubstituted or substituted aryl;
Q¹ is C═O, C═S, C═CR¹⁴R¹⁵, C═NR¹⁴, alkylene, alkenylene, or alkynylene, each of which is independently substituted or unsubstituted, or a bond;
n is 0, 1, 2, 3, or 4;
each R^{x} and R¹ is independently C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted; cyano, halo, or hydrogen; or R¹ and R^{x} together with Ar form a fused ring;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

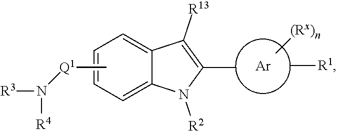

wherein the variables are as defined above.

In some embodiments, Ar is a 6-carbon monocyclic or 10-carbon bicyclic aromatic ring system wherein 0, 1, 2, 3, or 4 atoms of each ring are optionally substituted. In some embodiments, Ar is phenyl. In some embodiments, Ar is naphthyl. In some embodiments, Ar is indazolyl.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is alkyl substituted with —C(O)NR¹⁶R¹⁷. In some embodiments, R¹ is methyl substituted with —C(O)NR¹⁶R¹⁷. In some embodiments, R¹ is alkyl substituted with —C(O)OR¹⁶. In some embodiments, R¹ is methyl substituted with COOH.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

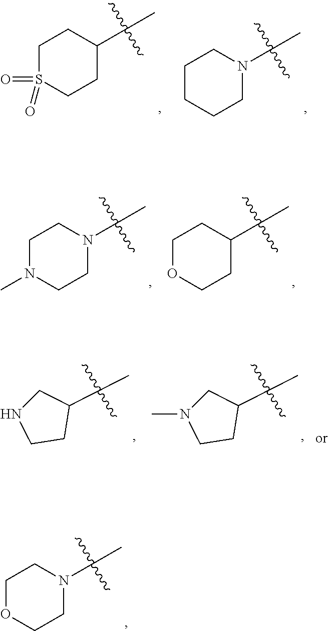

wherein the ring is substituted or unsubstituted. In some embodiments, R³ is H, and R⁴ is a ring that is

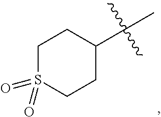 [SMILES: *C1CCS(=O)(=O)CC1]

wherein the ring is substituted or unsubstituted. In some embodiments, R³ is H, and R⁴ is a ring that is

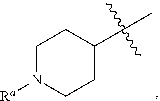

wherein the ring is substituted or unsubstituted. In some embodiments, R^{a} is alkylene. In some embodiments, R^{a} is methyl. In some embodiments, R³ is H, and R⁴ is a ring that is

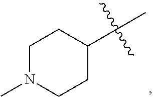 [SMILES: *C1CCN(C)CC1]

wherein the ring is substituted or unsubstituted. In some embodiments, R³ is H, and R⁴ is a ring that is

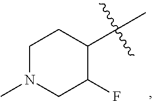 [SMILES: *C1CCN(C)CC1F]

wherein the ring is substituted or unsubstituted. In some embodiments, R³ is H, and R⁴ is a ring that is

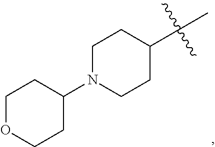 [SMILES: *C1CCN(C2CCOCC2)CC1]

In some embodiments, the disclosure provides a compound of the formula:

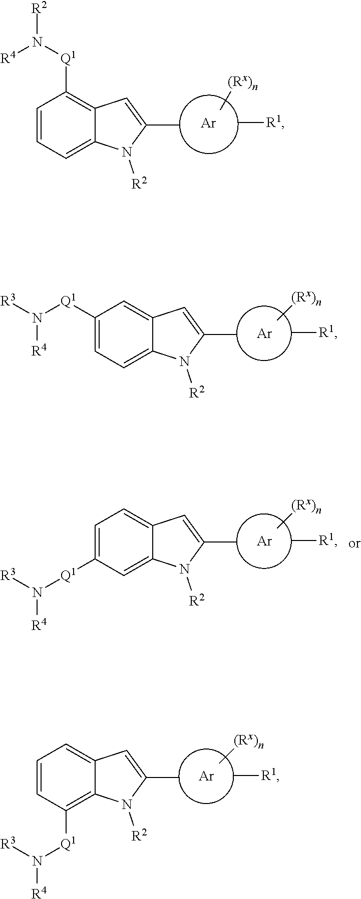

or a pharmaceutically-acceptable salt thereof, wherein the variables are as defined above.

In some embodiments, the disclosure provides a compound of the formula:

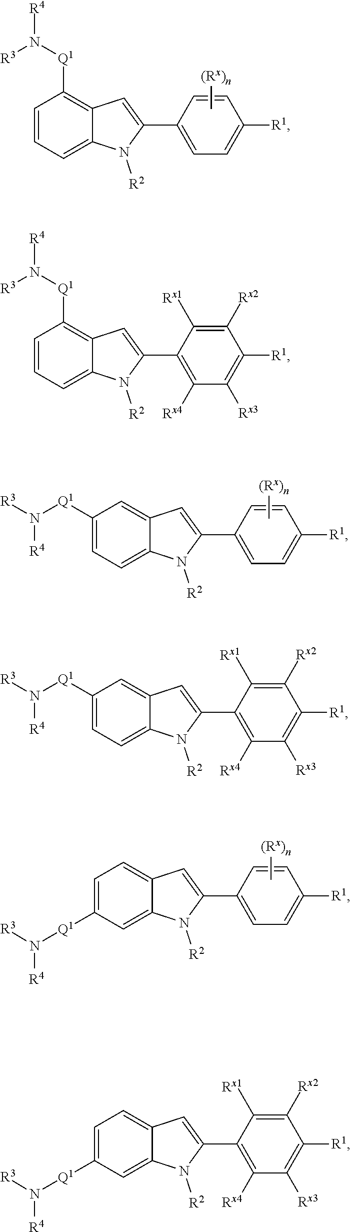

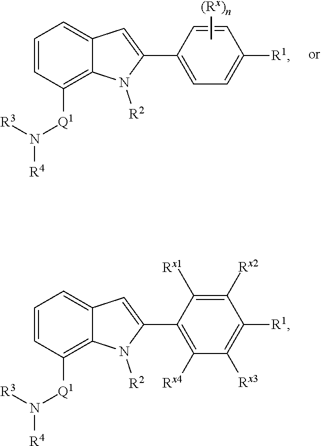

wherein:
Q¹ is C═O, C═S, C═CR¹⁴R¹⁵, C═NR¹⁴, alkylene, alkenylene, or alkynylene, each of which is independently substituted or unsubstituted, or a bond;
each R¹, R^{x}, R^{x1}, R^{x2}, R^{x3}, and R^{x4} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted; cyano, halo, or hydrogen; or R¹ and R^{x} together with Ar form a fused ring;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
n is 0, 1, 2, 3, or 4;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is methyl substituted with NR¹⁶R¹⁷, wherein R¹⁶ is hydrogen, and R¹⁷ is alkyl, aryl, heteroaryl, an amino group, a carboxyl group, or an ester group, any of which is substituted or unsubstituted. In some embodiments, R¹ is methyl substituted with NR¹⁶R¹⁷, wherein R¹⁶ is hydrogen, and R¹⁷ is substituted or unsubstituted alkyl, aryl, or heteroaryl. In some embodiments, R¹ is methyl substituted with NR¹⁶R¹⁷, wherein R¹⁶ is hydrogen, and R¹⁷ is substituted or unsubstituted phenyl. In some embodiments, R¹ is methyl substituted with NR¹⁶R¹⁷, wherein R¹⁶ is hydrogen, and R¹⁷ is substituted or unsubstituted pyridinyl.

In some embodiments, R¹ is —C(O)NR¹⁶R¹⁷. In some embodiments, R¹ is —C(O)NR¹⁶R¹⁷, wherein R¹⁶ and R¹⁷ are hydrogen. In some embodiments, R¹ is —C(O)NR¹⁶R¹⁷, wherein R¹⁶ is hydrogen, and R¹⁷ alkyl. In some embodiments, R¹ is —C(O)NR¹⁶R¹⁷, wherein R¹⁶ is hydrogen, and R¹⁷ methyl. In some embodiments, R¹ is —C(O)OR¹⁶. In some embodiments, R¹ is —C(O)OH. In some embodiments, R¹ is methyl. In some embodiments, R¹ is halogen. In some embodiments, R¹ is chloro or fluoro.

In some embodiments, n is 0, 1, 2, or 3. In some embodiments, n is 1. In some embodiments, n is 2. In some embodiments, n is 0.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

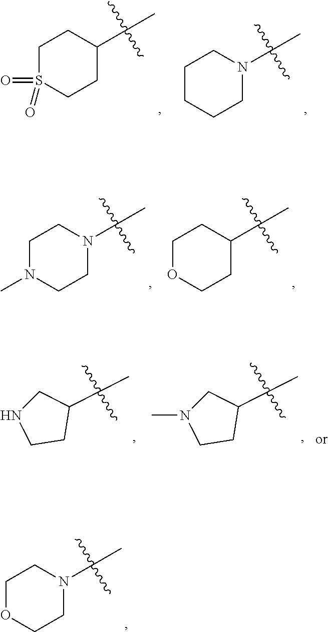

wherein the ring is substituted or unsubstituted. In some embodiments, R³ is H, and R⁴ is a ring that is

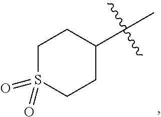 [SMILES: *C1CCS(=O)(=O)CC1]

wherein the ring is substituted or unsubstituted. In some embodiments, R³ is H, and R⁴ is a ring that is

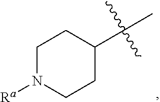

wherein the ring is substituted or unsubstituted. In some embodiments, R^{a} is alkylene. In some embodiments, R^{a} is methyl. In some embodiments, R³ is H, and R⁴ is a ring that is

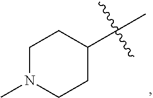 [SMILES: *C1CCN(C)CC1]

wherein the ring is substituted or unsubstituted. In some embodiments, R³ is H, and R⁴ is a ring that is

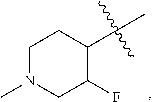 [SMILES: *C1CCN(C)CC1F]

wherein the ring is substituted or unsubstituted. In some embodiments, R³ is H, and R⁴ is a ring that is

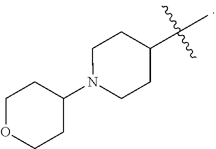 [SMILES: *C1CCN(C2CCOCC2)CC1]

In some embodiments, the disclosure provides a compound of the formula:

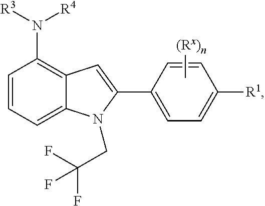

or a pharmaceutically-acceptable salt thereof, wherein the variables are as defined above.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is methyl substituted with NR¹⁶R¹⁷, wherein R¹⁶ is hydrogen, and R¹⁷ is alkyl, aryl, heteroaryl, an amino group, a carboxyl group, or an ester group, any of which is substituted or unsubstituted. In some embodiments, R¹ is methyl substituted with NR¹⁶R¹⁷, wherein R¹⁶ is hydrogen, and R¹⁷ is substituted or unsubstituted alkyl, aryl, or heteroaryl. In some embodiments, R¹ is methyl substituted with NR¹⁶R¹⁷, wherein R¹⁶ is hydrogen, and R¹⁷ is substituted or unsubstituted phenyl. In some embodiments, R¹ is methyl substituted with NR¹⁶R¹⁷, wherein R¹⁶ is hydrogen, and R¹⁷ is substituted or unsubstituted pyridinyl.

In some embodiments, R¹ is —C(O)NR¹⁶R¹⁷. In some embodiments, R¹ is —C(O)NR¹⁶R¹⁷, wherein R¹⁶ and R¹⁷ are hydrogen. In some embodiments, R¹ is —C(O)NR¹⁶R¹⁷, wherein R¹⁶ is hydrogen, and R¹⁷ alkyl. In some embodiments, R¹ is —C(O)NR¹⁶R¹⁷, wherein R¹⁶ is hydrogen, and R¹⁷ methyl. In some embodiments, R¹ is —C(O)OR¹⁶. In some embodiments, R¹ is —C(O)OH. In some embodiments, R¹ is methyl. In some embodiments, R¹ is halogen. In some embodiments, R¹ is chloro or fluoro.

In some embodiments, n is 1, 2, or 3. In some embodiments, n is 1. In some embodiments, n is 2. In some embodiments, n is 0.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R³ is H, and R⁴ is a ring that is:

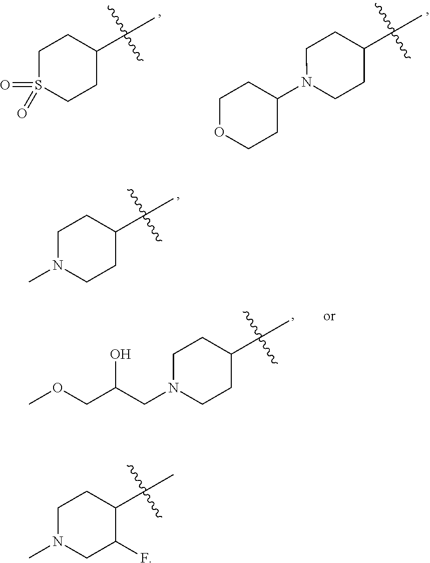

In some embodiments, R³ is H, and R⁴ is a ring that is

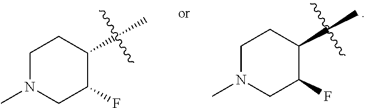

In some embodiments, R³ is H, and R⁴ is a ring that is

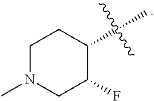 [SMILES: *[C@H]1CCN(C)C[C@H]1F]

Non-limiting examples of compounds of the disclosure include compounds of any of the following formulae:

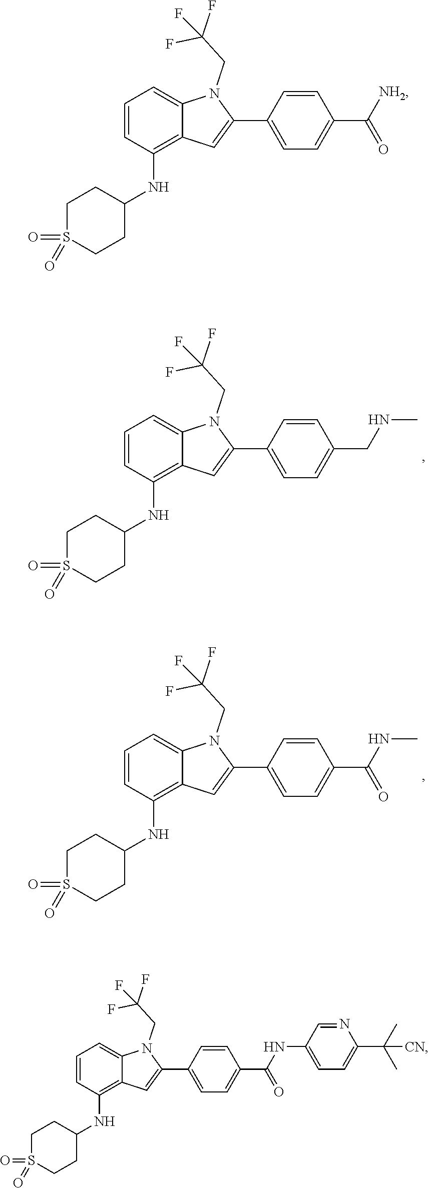 [SMILES: NC(=O)c1ccc(-c2cc3c(NC4CCS(=O)(=O)CC4)cccc3n2CC(F)(F)F)cc1 ; CNCc1ccc(-c2cc3c(NC4CCS(=O)(=O)CC4)cccc3n2CC(F)(F)F)cc1 ; CNC(=O)c1ccc(-c2cc3c(NC4CCS(=O)(=O)CC4)cccc3n2CC(F)(F)F)cc1 ; CC(C)(C#N)c1ccc(NC(=O)c2ccc(-c3cc4c(NC5CCS(=O)(=O)CC5)cccc4n3CC(F)(F)F)cc2)cn1]

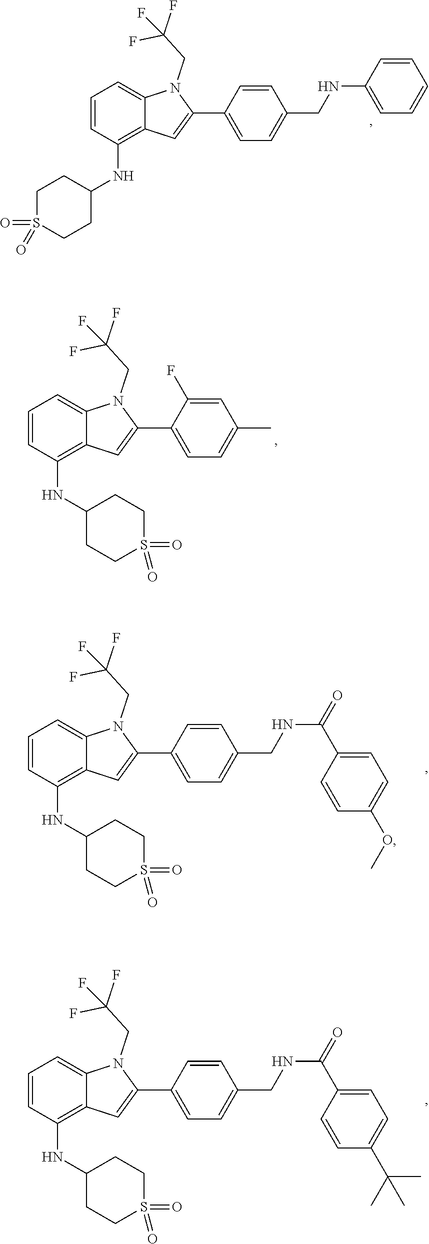 [SMILES: O=S1(=O)CCC(Nc2cccc3c2cc(-c2ccc(CNc4ccccc4)cc2)n3CC(F)(F)F)CC1 ; Cc1ccc(-c2cc3c(NC4CCS(=O)(=O)CC4)cccc3n2CC(F)(F)F)c(F)c1 ; COc1ccc(C(=O)NCc2ccc(-c3cc4c(NC5CCS(=O)(=O)CC5)cccc4n3CC(F)(F)F)cc2)cc1 ; CC(C)(C)c1ccc(C(=O)NCc2ccc(-c3cc4c(NC5CCS(=O)(=O)CC5)cccc4n3CC(F)(F)F)cc2)cc1]

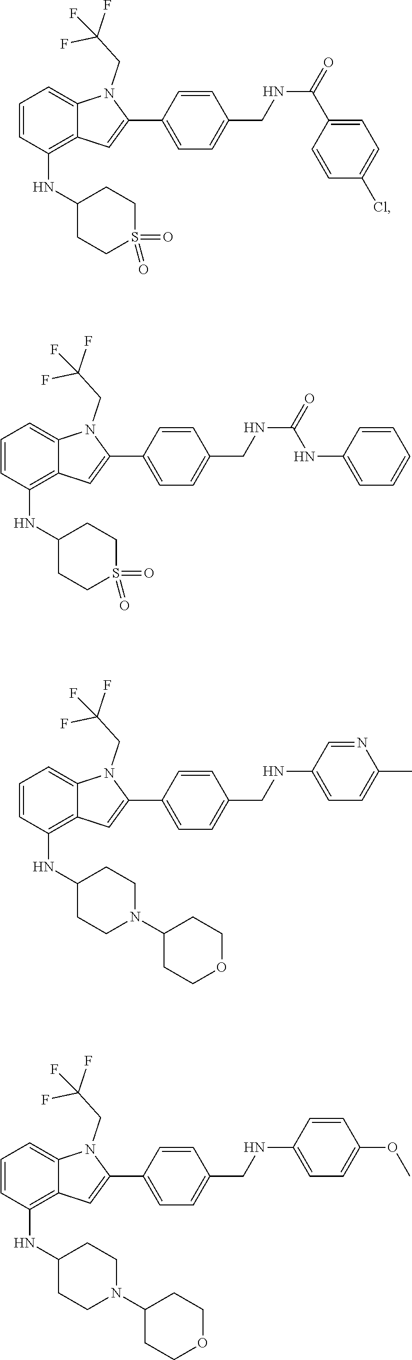 [SMILES: O=C(NCc1ccc(-c2cc3c(NC4CCS(=O)(=O)CC4)cccc3n2CC(F)(F)F)cc1)c1ccc(Cl)cc1 ; O=C(NCc1ccc(-c2cc3c(NC4CCS(=O)(=O)CC4)cccc3n2CC(F)(F)F)cc1)Nc1ccccc1 ; Cc1ccc(NCc2ccc(-c3cc4c(NC5CCN(C6CCOCC6)CC5)cccc4n3CC(F)(F)F)cc2)cn1 ; COc1ccc(NCc2ccc(-c3cc4c(NC5CCN(C6CCOCC6)CC5)cccc4n3CC(F)(F)F)cc2)cc1]

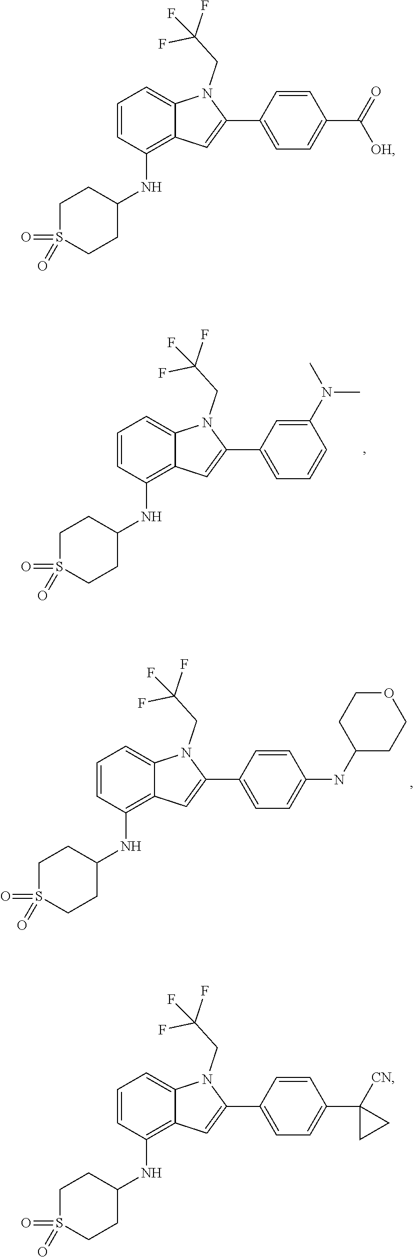 [SMILES: O=C(O)c1ccc(-c2cc3c(NC4CCS(=O)(=O)CC4)cccc3n2CC(F)(F)F)cc1 ; CN(C)c1cccc(-c2cc3c(NC4CCS(=O)(=O)CC4)cccc3n2CC(F)(F)F)c1 ; O=S1(=O)CCC(Nc2cccc3c2cc(-c2ccc(NC4CCOCC4)cc2)n3CC(F)(F)F)CC1 ; N#CC1(c2ccc(-c3cc4c(NC5CCS(=O)(=O)CC5)cccc4n3CC(F)(F)F)cc2)CC1]

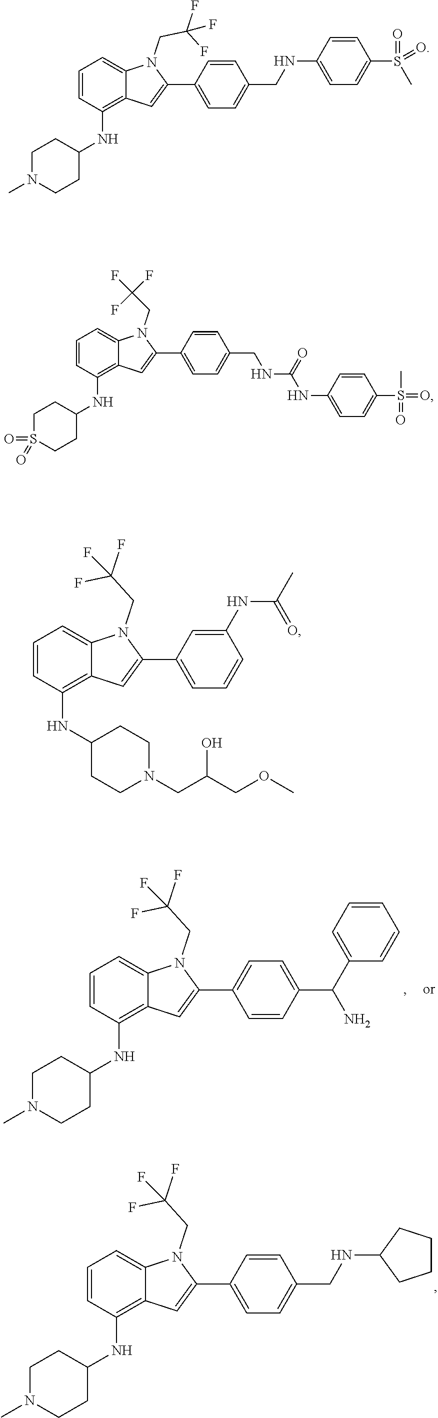

or a pharmaceutically-acceptable salt thereof.

In some embodiments, the disclosure provides a compound of the formula:

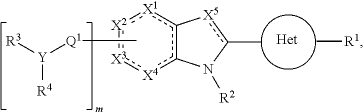

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Het is substituted or unsubstituted heteroaryl;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

The pattern of dashed bonds can be chosen to provide an aromatic system, for example, an indole, an indolene, a pyrrolopyridine, a pyrrolopyrimidine, or a pyrrolopyrazine. In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Het is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, Het is an aromatic 8-membered, 9-membered, 10-membered, 11-membered, or 12-membered bicyclic ring system comprising 1, 2, 3, 4, 5, or 6 heteroatoms, wherein each heteroatom is independently selected from O, N, or S. In some embodiments, Het is an aromatic 5-membered, 6-membered, 7-membered, or 8-membered monocyclic ring system comprising 1, 2, or 3 heteroatoms, and the aromatic 5-membered, 6-membered, 7-membered, or 8-membered monocyclic ring system is substituted. In some embodiments, Het is an 8-membered, 9-membered, 10-membered, 11-membered, or 12-membered bicyclic ring system having 1, 2, 3, 4, 5, or 6 heteroatoms, and the 8-membered, 9-membered, 10-membered, 11-membered, or 12-membered bicyclic ring system is substituted.

In some embodiments, Het is pyridinyl, pyrimidinyl, thiadiazolyl, thiazolyl, pyrazolyl, thiophenyl, or oxadiazolyl, each of which is independently substituted or unsubstituted. In some embodiments, Het is 1,3,5-thiadiazol-2-yl. In some embodiments, Het is 1,3,4-oxadiazol-2-yl or 1,2,4-oxadiazol-2-yl. In some embodiments, Het is 1,3,4-oxadiazol-2-yl.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen. In some embodiments, R¹ is methyl, cyclohexyl, methylene, methoxy, or benzyl. In some embodiments, R¹ is fluoro or chloro. In some embodiments, R¹ is phenyl. In some embodiments, R¹ is hydrogen.

In some embodiments, R¹ is a substituted alkyl or alkylene. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is substituted alkyl. In some embodiments, R¹ is alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, m is 1, 2, 3, or 4. In some embodiments, m is 1. In some embodiments, X¹ is carbon atom connected to Q¹, and m is 1. In some embodiments, X² is carbon atom connected to Q¹, and m is 1.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, Q¹ is C₁-alkylene, R¹⁶ is aryl, and R¹⁷ is alkyl. In some embodiments, Q¹ is C₁-alkylene, R¹⁶ is aryl, and R¹⁷ is hydrogen. In some embodiments, Q¹ is C₁-alkylene, R¹⁶ is heteroaryl, and R¹⁷ is alkyl. In some embodiments, Q¹ is C₁-alkylene, R¹⁶ is heteroaryl, and R¹⁷ is hydrogen. In some embodiments, Q¹ is C₁-alkylene, R¹⁶ is substituted heteroaryl, and R¹⁷ is hydrogen. In some embodiments, Q¹ is C₁-alkylene, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano.

In some embodiments, R² is hydrogen or alkyl. In some embodiments, R² is substituted alkyl. In some embodiments, R² is trifluoroethyl. In some embodiments, R¹³ is alkyl, alkenyl, hydrogen, or halogen. In some embodiments, R¹³ is methyl, ethyl, propyl, iso-propyl, butyl or tert-butyl. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen; and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

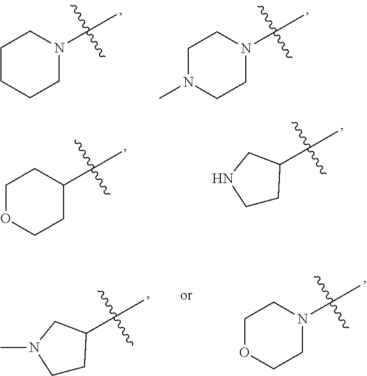

wherein the ring is substituted or unsubstituted. In some embodiments, R³ is H, and R⁴ is a ring that is

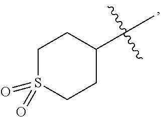 [SMILES: *C1CCS(=O)(=O)CC1]

wherein the ring is substituted or unsubstituted. In some embodiments, R³ is H, and R⁴ is a ring that is

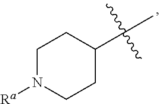

wherein the ring is substituted or unsubstituted. In some embodiments, R^{a} is alkylene. In some embodiments, R^{a} is methyl. In some embodiments, R³ is H, and R⁴ is a ring that is

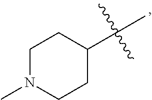 [SMILES: *C1CCN(C)CC1]

wherein the ring is substituted or unsubstituted. In some embodiments, R³ is H, and R⁴ is a ring that is

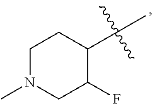 [SMILES: *C1CCN(C)CC1F]

wherein the ring is substituted or unsubstituted.

In some embodiments, R³ and R⁴ together with the nitrogen atom to which R³ and R⁴ are bound form a ring, wherein the ring is substituted or unsubstituted. In some embodiments, R³ and R⁴ together with the nitrogen atom to which R³ and R⁴ are bound form a substituted heterocycle. In some embodiments, R³ and R⁴ together with the nitrogen atom to which R³ and R⁴ are bound form a heterocycle substituted with a hydroxyl group, halogen, amino group, or alkyl group. In some embodiments, R³ and R⁴ together with the nitrogen atom to which R³ and R⁴ are bound form a heterocycle, wherein the heterocycle is substituted by a substituted or unsubstituted heterocycle.

In some embodiments, the disclosure provides a compound of the formula:

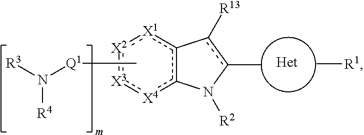

or a pharmaceutically-acceptable salt thereof, wherein the variables are as defined above.

In some embodiments, the disclosure provides a compound of the formula:

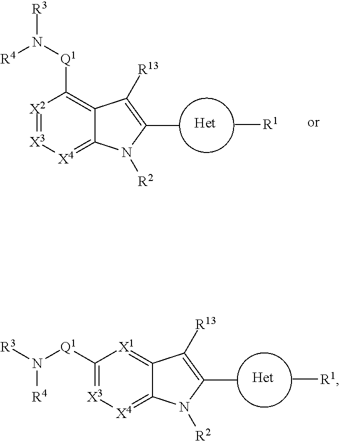

or a pharmaceutically-acceptable salt thereof, wherein the variables are as defined above.

In some embodiments, the compound is of the formula:

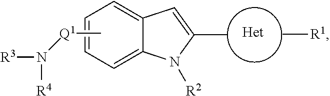

or a pharmaceutically-acceptable salt thereof, wherein the variables are as defined above.

In some embodiments, the disclosure provides a compound of the formula:

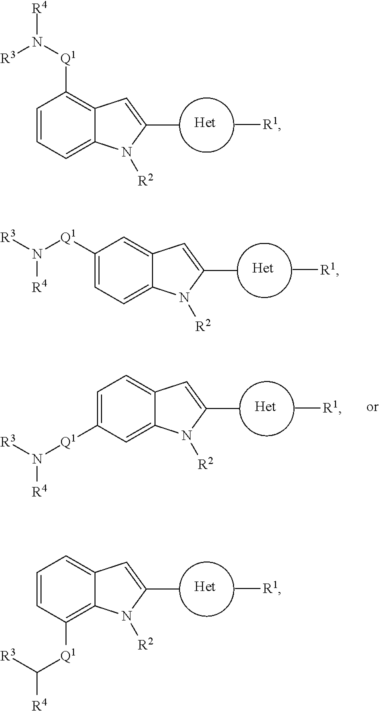

or a pharmaceutically-acceptable salt thereof, wherein the variables are as defined above.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen.

In some embodiments, R¹ is substituted alkyl. In some embodiments, R¹ is alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, R² is hydrogen or alkyl. In some embodiments, R² is substituted alkyl. In some embodiments, R² is trifluoroethyl.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

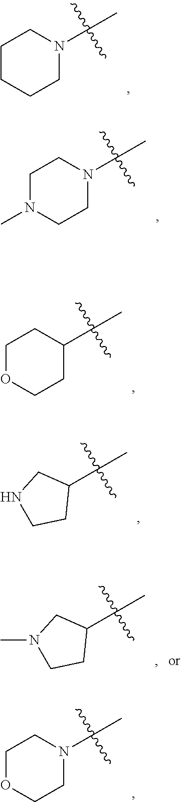

wherein the ring is substituted or unsubstituted. In some embodiments, R is H, and R⁴ is a ring that is

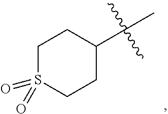 [SMILES: *C1CCS(=O)(=O)CC1]

wherein the ring is substituted or unsubstituted. In some embodiments, R³ is H, and R⁴ is a ring that is

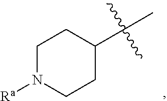

wherein the ring is substituted or unsubstituted. In some embodiments, R^{a} is alkylene. In some embodiments, R^{a} is methyl. In some embodiments, R³ is H, and R⁴ is a ring that is

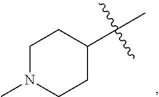 [SMILES: *C1CCN(C)CC1]

wherein the ring is substituted or unsubstituted. In some embodiments, R³ is H, and R⁴ is a ring that is

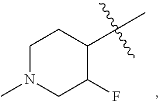 [SMILES: *C1CCN(C)CC1F]

wherein the ring is substituted or unsubstituted.

In some embodiments, the disclosure provides a compound of the formula:

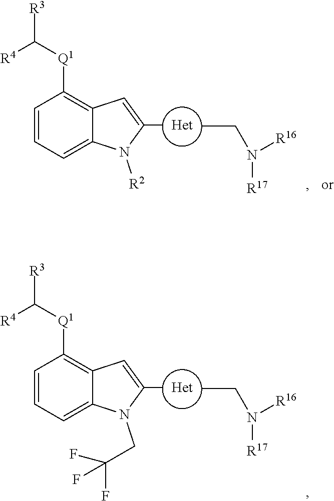

or a pharmaceutically-acceptable salt thereof, wherein the variables are as defined above.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that

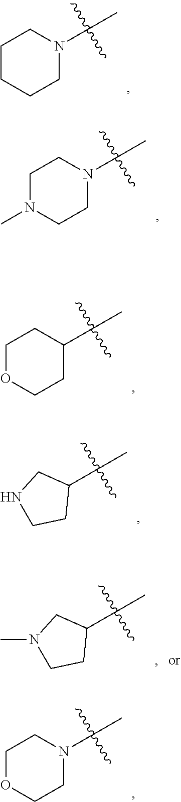

wherein the ring is substituted or unsubstituted. In some embodiments, R³ is H, and R⁴ is a ring that is

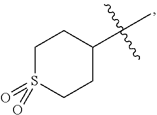 [SMILES: *C1CCS(=O)(=O)CC1]

wherein the ring is substituted or unsubstituted. In some embodiments, R³ is H, and R⁴ is a ring that is

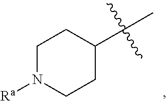

wherein the ring is substituted or unsubstituted. In some embodiments, R^{a} is alkylene. In some embodiments, R^{a} is methyl. In some embodiments, R³ is H, and R⁴ is a ring that is

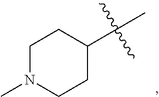 [SMILES: *C1CCN(C)CC1]

wherein the ring is substituted or unsubstituted. In some embodiments, R³ is H, and R⁴ is a ring that is

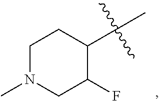 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

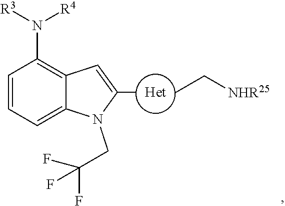

wherein R²⁵ is —C(O)R¹⁶, —C(O)NR¹⁶R¹⁷, alkyl, alkenyl, alkynyl, aryl, heteroaryl, or heterocyclyl, each of which is independently substituted or unsubstituted, or hydrogen. In some embodiments, R²⁵ is aryl that is substituted or unsubstituted. In some embodiments, R²⁵ is substituted phenyl. In some embodiments, R²⁵ is —C(O)R¹⁶, wherein R¹⁶ is alkyl, aryl, heteroaryl, or heterocyclyl. In some embodiments, R²⁵ is —C(O)R¹⁶, wherein R¹⁶ is substituted phenyl; or a pharmaceutically-acceptable salt thereof,

In some embodiments, the compound is of the formula:

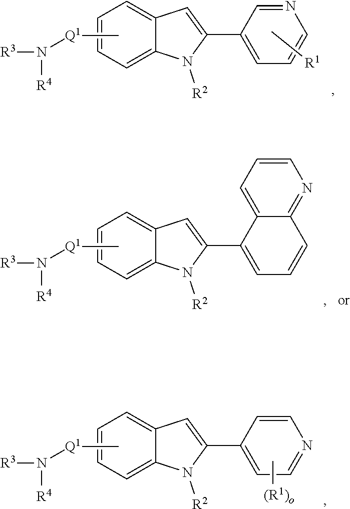

wherein:
Q¹ is C═O, C═S, C═CR¹⁴R¹⁵, C═NR¹⁴, alkylene, alkenylene, or alkynylene, each of which is independently substituted or unsubstituted, or a bond;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.
   the variables are as defined above, and wherein o is 1, 2, 3, or 4.

In some embodiments, the compound is of the formula:

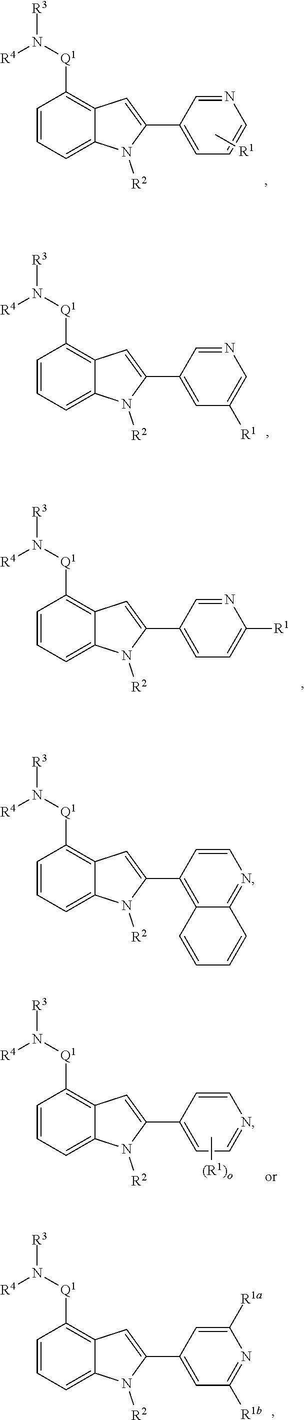

wherein:
Q¹ is C═O, C═S, C═CR¹⁴R¹⁵, C═NR¹⁴, alkylene, alkenylene, or alkynylene, each of which is independently substituted or unsubstituted, or a bond;
each R¹, R^{1a}, and R^{1b} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
o is 0, 1, 2, 3, or 4;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, each R^{1a} and R^{1b} is independently alkyl, alkoxy, aryl, heteroaryl, heterocyclyl, or NR¹⁶R¹⁷. In some embodiments, R^{1a} is unsubstituted phenyl, and R^{1b} is amino.

In some embodiments, the compound is of the formula:

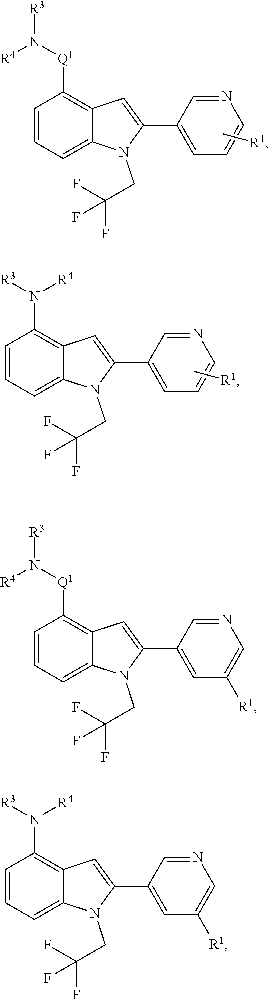

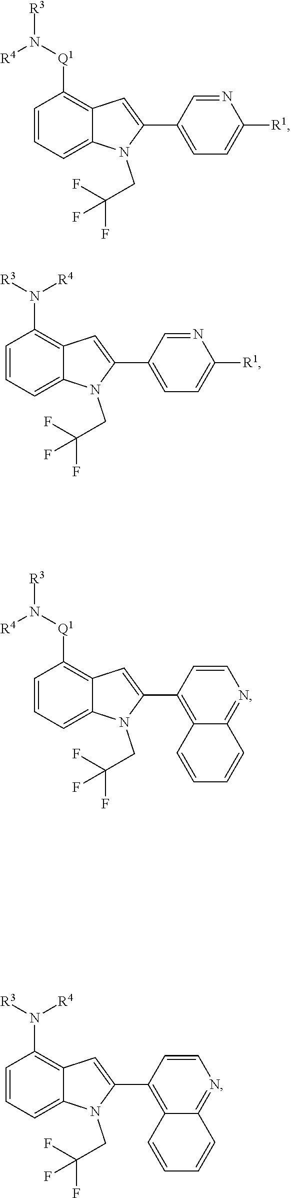

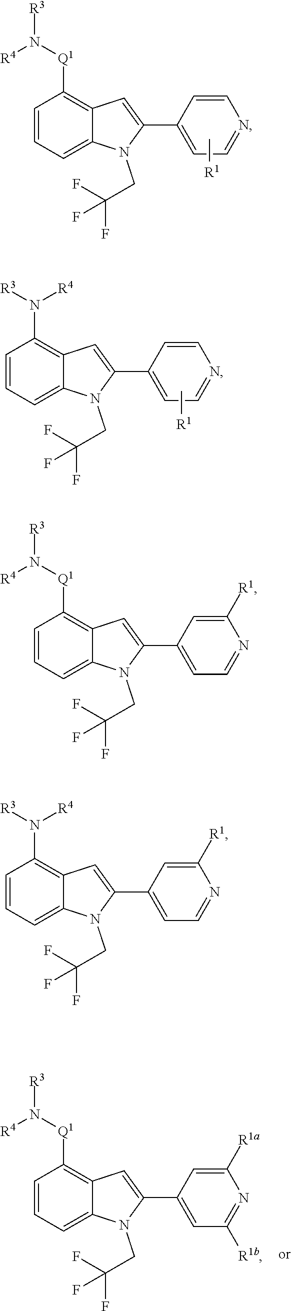

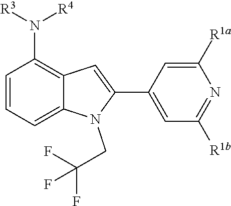

or a pharmaceutically-acceptable salt thereof, wherein the variables are as defined above.

In some embodiments, R¹ is —C(O)NR¹⁶R¹⁷, alkyl, alkenyl, alkynyl, alkoxy, aryl, heteroaryl, heterocyclyl, or halo, each of which is independently substituted or unsubstituted, or hydrogen. In some embodiments, R¹ is alkyl, alkoxy, aryl, or halo. In some embodiments, R¹ is methoxy, methyl, or phenyl. In some embodiments, each R^{1a} and R^{1b} is independently alkyl, alkoxy, aryl, heteroaryl, heterocyclyl, or NR¹⁶R¹⁷. In some embodiments, R^{1a} is unsubstituted phenyl, and R^{1b} is amino.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

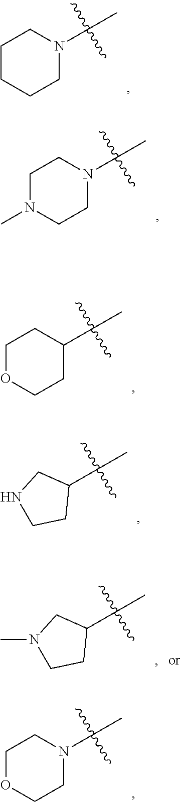

wherein the ring is substituted or unsubstituted. In some embodiments, R³ is H, and R⁴ is a ring that is

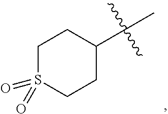 [SMILES: *C1CCS(=O)(=O)CC1]

wherein the ring is substituted or unsubstituted. In some embodiments, R³ is H, and R⁴ is a ring that is

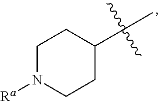

wherein the ring is substituted or unsubstituted. In some embodiments, R^{a} is alkylene. In some embodiments, R^{a} is methyl. In some embodiments, R³ is H, and R⁴ is a ring that is

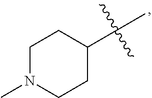 [SMILES: *C1CCN(C)CC1]

wherein the ring is substituted or unsubstituted. In some embodiments, R³ is H, and R⁴ is a ring that is

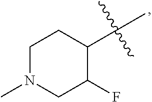 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

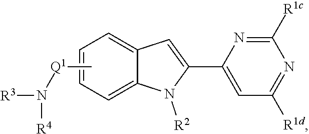

wherein:
Q¹ is C═O, C═S, C═CR¹⁴R¹⁵, C═NR¹⁴, alkylene, alkenylene, or alkynylene, each of which is independently substituted or unsubstituted, or a bond;
each R^{1c} and R^{1d} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, each R^{1c} and R^{1d} is independently —OR¹⁶, —NR¹⁶R¹⁷, —NR¹⁶C(O)R¹⁶, alkyl, alkenyl, alkynyl, alkoxy, aryl, heteroaryl, or heterocyclyl, each of which is independently substituted or unsubstituted, or hydrogen.

In some embodiments, the compound is of the formula:

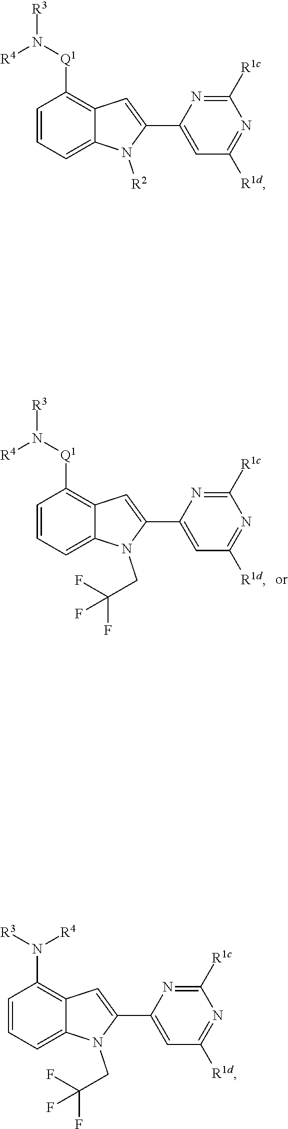

or a pharmaceutically-acceptable salt thereof, wherein the variables are as defined above.

In some embodiments, each R^{1c} and R^{1d} is independently C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen. In some embodiments, R^{1c} is amino, and R^{1d} is phenyl. In some embodiments, R^{1c} is amino, and R^{1d} is cycloalkenyl.

In some embodiments, the compound is of the formula:

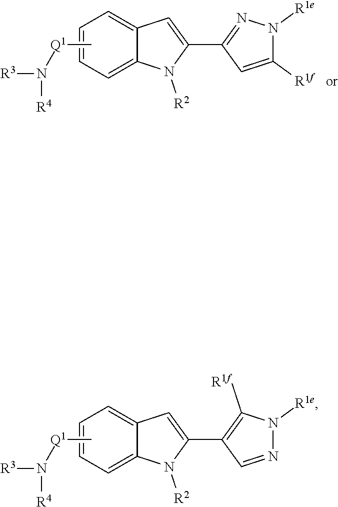

wherein:
Q¹ is C═O, C═S, C═CR¹⁴R¹⁵, C═NR¹⁴, alkylene, alkenylene, or alkynylene, each of which is independently substituted or unsubstituted, or a bond;
each R^{1e} and R^{1f} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

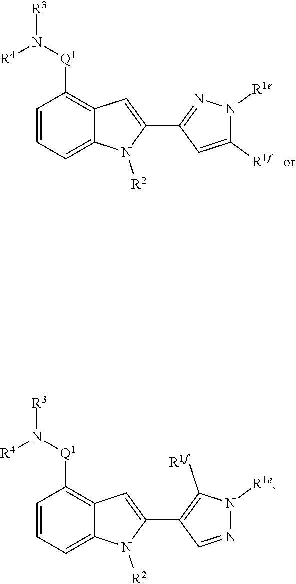

or a pharmaceutically-acceptable salt thereof, wherein the variables are as defined above.

In some embodiments, the compound is of the formula:

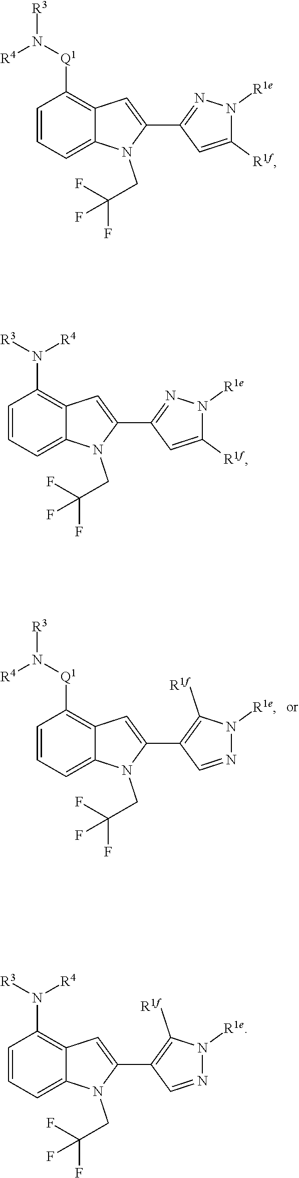

or a pharmaceutically-acceptable salt thereof, wherein the variables are as defined above.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

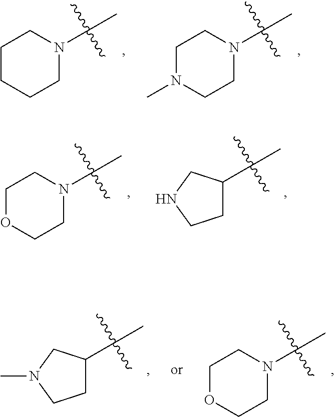

wherein the ring is substituted or unsubstituted. In some embodiments, R³ is H, and R⁴ is a ring that is

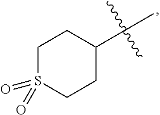 [SMILES: *C1CCS(=O)(=O)CC1]

wherein the ring is substituted or unsubstituted. In some embodiments, R³ is H, and R⁴ is a ring that is

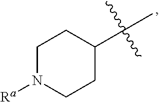

wherein the ring is substituted or unsubstituted. In some embodiments, R^{a} is alkylene. In some embodiments, R^{a} is methyl. In some embodiments, R³ is H, and R⁴ is a ring that is

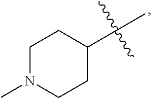 [SMILES: *C1CCN(C)CC1]

wherein the ring is substituted or unsubstituted. In some embodiments, R³ is H, and R⁴ is a ring that is

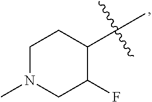 [SMILES: *C1CCN(C)CC1F]

wherein the ring is substituted or unsubstituted.

In some embodiments, each R^{1e} and R^{1f} is independently alkyl, NR¹⁶R¹⁷, aryl, heteroaryl, or heterocyclyl, each of which is independently substituted or unsubstituted, or hydrogen. In some embodiments, R^{1e} is substituted alkyl, and R^{1f} is hydrogen. In some embodiments, R^{1e} is hydrogen, and R^{1f} is NR¹⁶R¹⁷, wherein each R¹⁶ and R¹⁷ is independently alkyl, alkenyl, aryl, heteroaryl, or heterocyclyl, each of which is independently substituted or unsubstituted, or hydrogen. In some embodiments, R^{1e} is hydrogen, and R^{1f} is NR¹⁶R¹⁷, wherein R¹⁶ is hydrogen, and R¹⁷ is alkyl. In some embodiments, R^{1e} is hydrogen, and R^{1f} is NR¹⁶R¹⁷, wherein R¹⁶ is hydrogen, and R¹⁷ is phenyl. In some embodiments, R^{1e} is hydrogen, and R^{1f} is amino.

In some embodiments, the compound is of the formula:

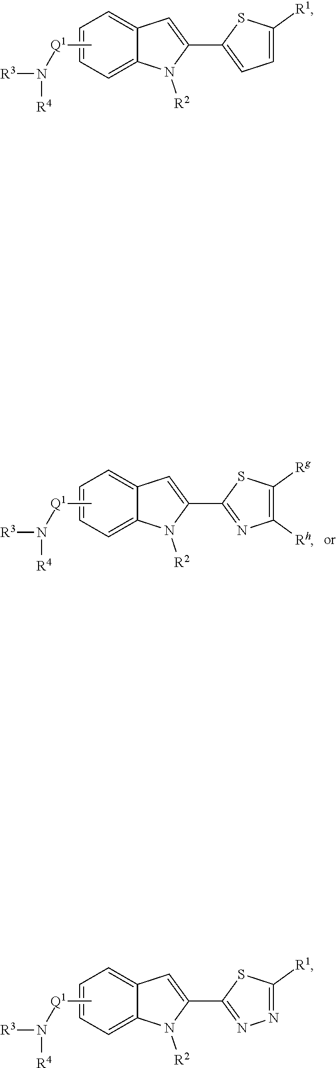

wherein:
Q¹ is C═O, C═S, C═CR¹⁴R¹⁵, C═NR¹⁴, alkylene, alkenylene, or alkynylene, each of which is independently substituted or unsubstituted, or a bond;
each R¹, R^{1g}, and R^{1h} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

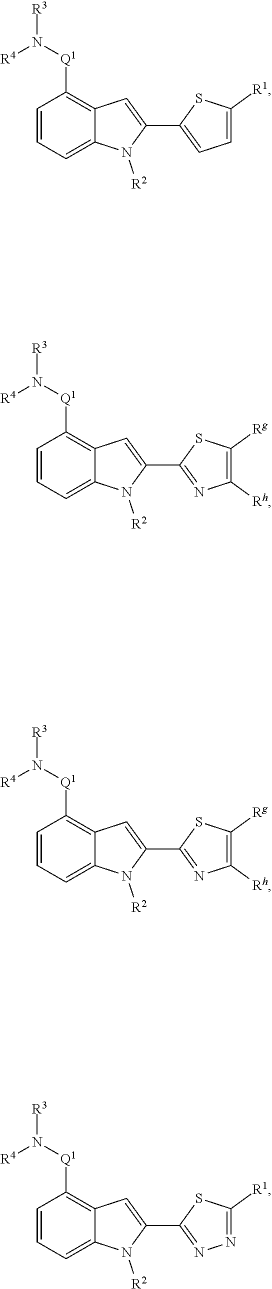

or a pharmaceutically-acceptable salt thereof, wherein the variables are as defined above.

In some embodiments, the compound is of the formula:

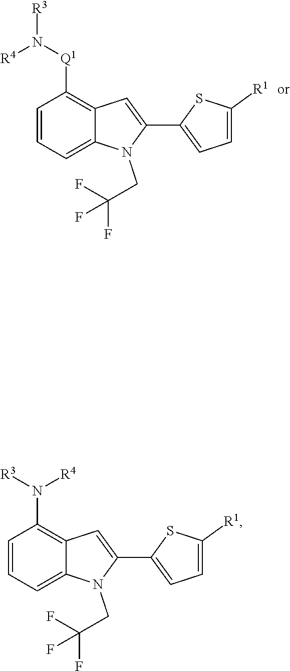

or a pharmaceutically-acceptable salt thereof, wherein the variables are as defined above.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

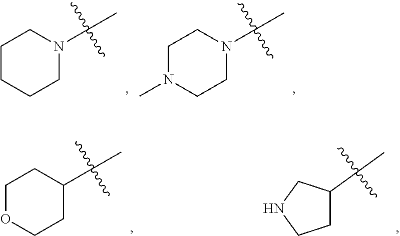

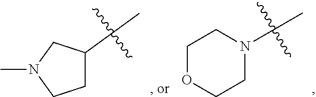

wherein the ring is substituted or unsubstituted. In some embodiments, R³ is H, and R⁴ is a ring that is

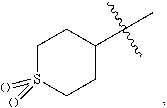 [SMILES: *C1CCS(=O)(=O)CC1]

wherein the ring is substituted or unsubstituted. In some embodiments, R³ is H, and R⁴ is a ring that is

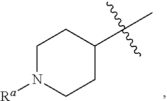

wherein the ring is substituted or unsubstituted. In some embodiments, R^{a} is alkylene. In some embodiments, R^{a} is methyl. In some embodiments, R³ is H, and R⁴ is a ring that is

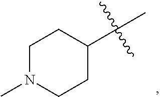 [SMILES: *C1CCN(C)CC1]

wherein the ring is substituted or unsubstituted. In some embodiments, R³ is H, and R⁴ is a ring that is

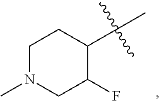 [SMILES: *C1CCN(C)CC1F]

wherein the ring is substituted or unsubstituted.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen. In some embodiments, R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group. In some embodiments, R¹⁶ is hydrogen, and R¹⁷ is carboxyl substituted with alkyl or aryl. In some embodiments, R¹⁶ is hydrogen, and R¹⁷ is carboxyl substituted with cycloalkyl or phenyl. In some embodiments, R¹⁶ and R¹⁷ are hydrogen.

In some embodiments, the compound is of the formula:

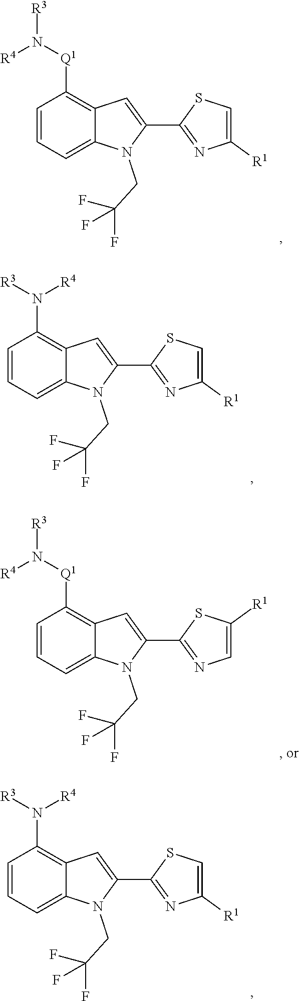

or a pharmaceutically-acceptable salt thereof, wherein the variables are as defined above.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen. In some embodiments, R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group. In some embodiments, R¹⁶ is hydrogen, and R¹⁷ is carboxyl substituted with alkyl or aryl. In some embodiments, R¹⁶ is hydrogen, and R¹⁷ is carboxyl substituted with cycloalkyl or phenyl. In some embodiments, R¹⁶ and R¹⁷ are hydrogen.

In some embodiments, the compounds is of the formula:

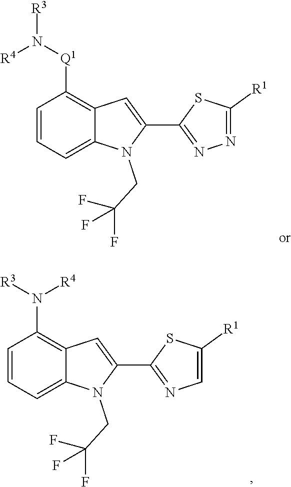

or a pharmaceutically-acceptable salt thereof, wherein the variables are as defined above.

In some embodiments, Q¹ is C═O, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

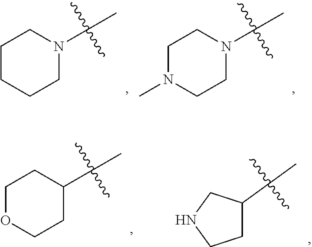

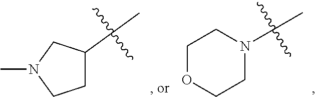

wherein the ring is substituted or unsubstituted. In some embodiments, R³ is H, and R⁴ is a ring that is

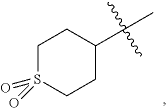 [SMILES: *C1CCS(=O)(=O)CC1]

wherein the ring is substituted or unsubstituted. In some embodiments, R³ is H, and R⁴ is a ring that is

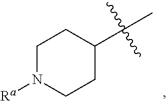

wherein the ring is substituted or unsubstituted. In some embodiments, R^{a} is alkylene. In some embodiments, R^{a} is methyl. In some embodiments, R³ is H, and R⁴ is a ring that is

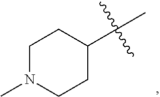 [SMILES: *C1CCN(C)CC1]

wherein the ring is substituted or unsubstituted. In some embodiments, R³ is H, and R⁴ is a ring that is

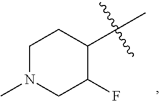 [SMILES: *C1CCN(C)CC1F]

wherein the ring is substituted or unsubstituted.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen. In some embodiments R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is aryl, heteroaryl, carboxyl, or hydrogen. In some embodiments, R¹⁶ is hydrogen, and R¹⁷ is carboxyl substituted with aryl, heteroaryl, cycloalkyl, or alkyl. In some embodiments, R¹⁶ and R¹⁷ are hydrogen.

In some embodiments, the compound is of the formula:

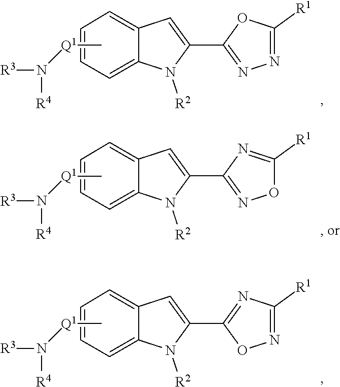

wherein:
Q¹ is C═O, C═S, C═CR¹⁴R¹⁵, C═NR¹⁴, alkylene, alkenylene, or alkynylene, each of which is independently substituted or unsubstituted, or a bond;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

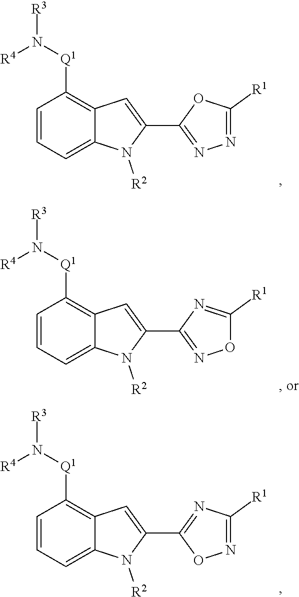

or a pharmaceutically-acceptable salt thereof, wherein the variables are as defined above.

In some embodiments, the compound is of the formula:

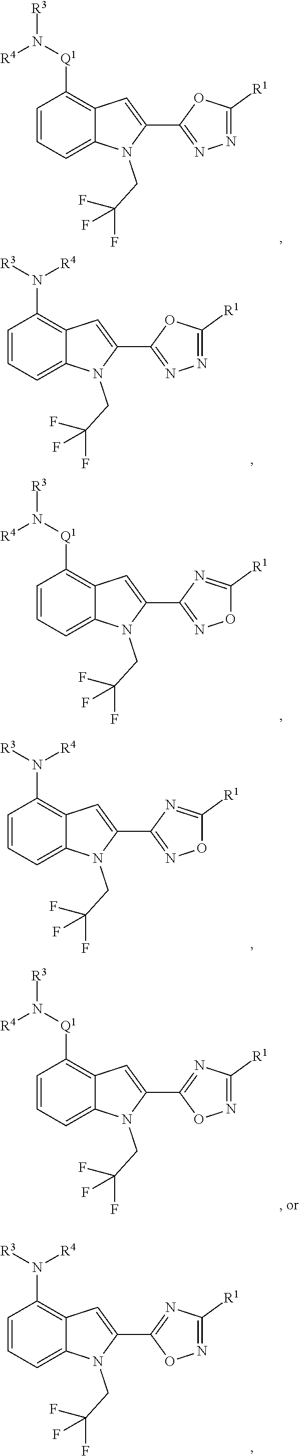

or a pharmaceutically-acceptable salt thereof, wherein the variables are as defined above.

In some embodiments, the compound is of the formula:

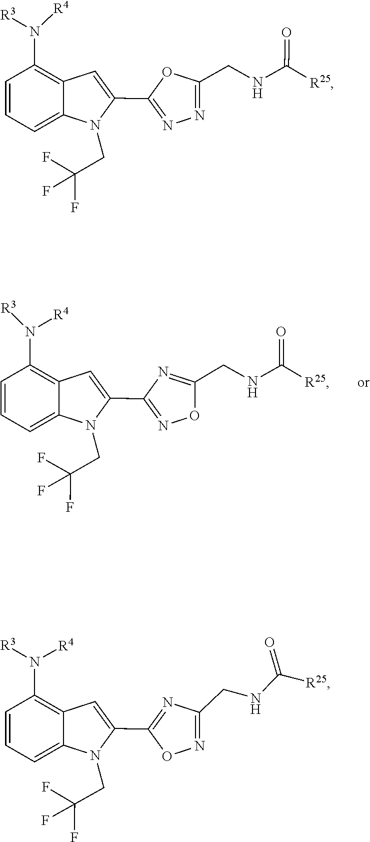

wherein:
Q¹ is C═O, C═S, C═CR¹⁴R¹⁵, C═NR¹⁴, alkylene, alkenylene, or alkynylene, each of which is independently substituted or unsubstituted, or a bond;
each R^{1c} and R^{1d} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, R²⁵ is alkyl, alkenyl, alkynyl, aryl, heteroaryl, or heterocyclyl, each of which is independently substituted or unsubstituted, or hydrogen;
   or a pharmaceutically-acceptable salt thereof.

In some embodiments, R²⁵ is heterocyclyl, cycloalkyl, aryl, each of which is substituted or unsubstituted. In some embodiments, R²⁵ is phenyl or cyclopropyl, each of which is substituted or unsubstituted. In some embodiments, R²⁵ is substituted cyclopropyl. In some embodiments, R²⁵ is heteroaryl or heterocyclyl, each of which is substituted or unsubstituted. In some embodiments, R²⁵ is thiophenyl, indolenyl, or pyrrolyl, each of which is substituted or unsubstituted.

Non-limiting examples of compounds of the disclosure include compounds of any of the following formulae:

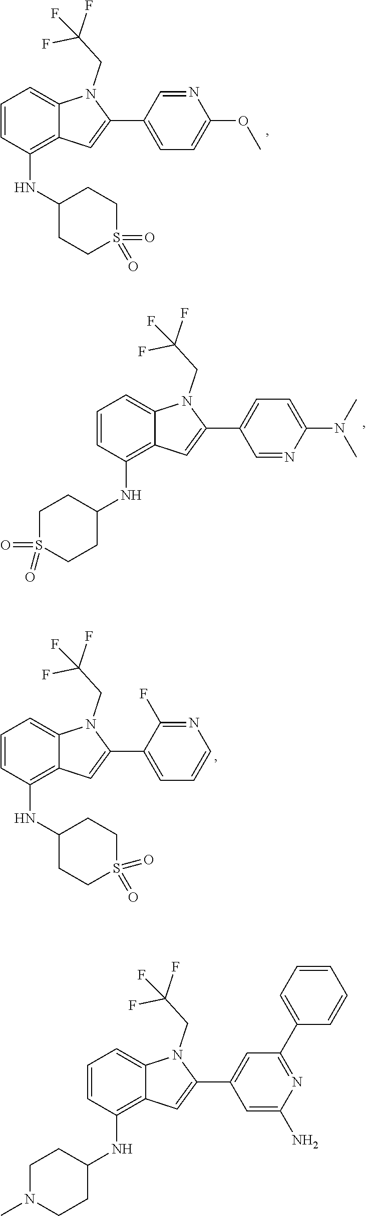

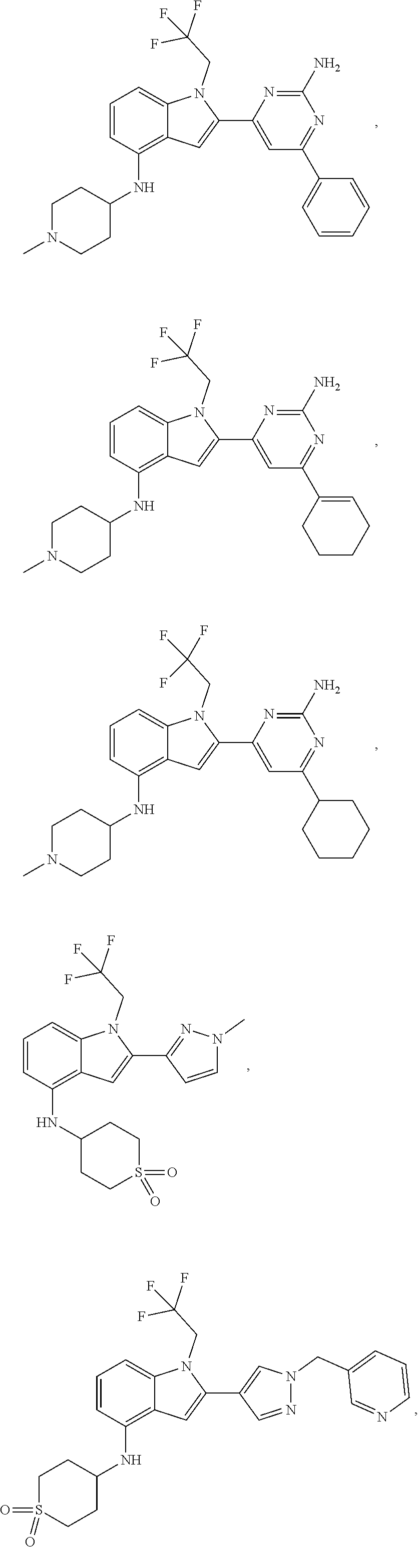 [SMILES: CN1CCC(Nc2cccc3c2cc(-c2cc(-c4ccccc4)nc(N)n2)n3CC(F)(F)F)CC1 ; CN1CCC(Nc2cccc3c2cc(-c2cc(C4=CCCCC4)nc(N)n2)n3CC(F)(F)F)CC1 ; CN1CCC(Nc2cccc3c2cc(-c2cc(C4CCCCC4)nc(N)n2)n3CC(F)(F)F)CC1 ; Cn1ccc(-c2cc3c(NC4CCS(=O)(=O)CC4)cccc3n2CC(F)(F)F)n1 ; O=S1(=O)CCC(Nc2cccc3c2cc(-c2cnn(Cc4cccnc4)c2)n3CC(F)(F)F)CC1]

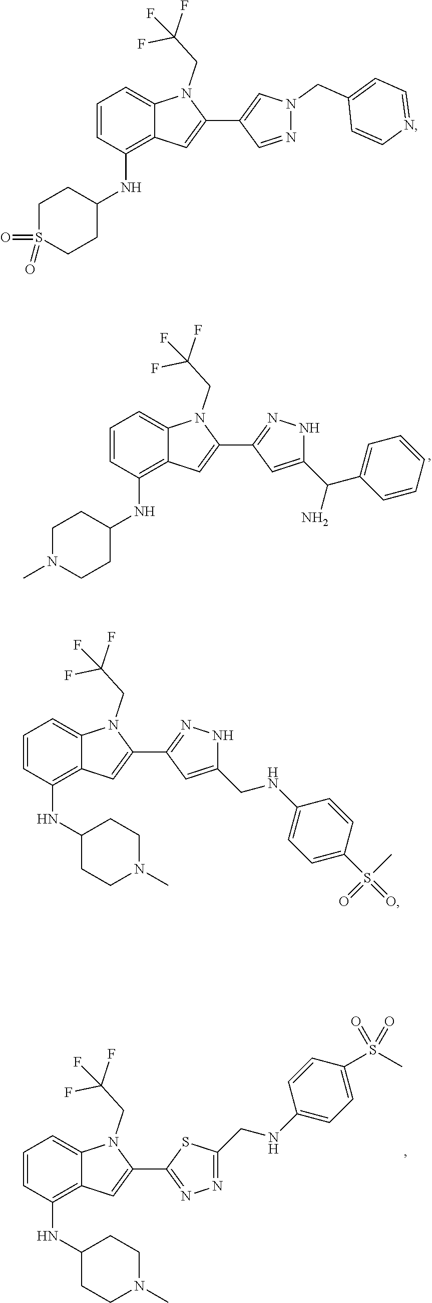 [SMILES: O=S1(=O)CCC(Nc2cccc3c2cc(-c2cnn(Cc4ccncc4)c2)n3CC(F)(F)F)CC1 ; CN1CCC(Nc2cccc3c2cc(-c2cc(C(N)c4ccccc4)[nH]n2)n3CC(F)(F)F)CC1 ; CN1CCC(Nc2cccc3c2cc(-c2cc(CNc4ccc(S(C)(=O)=O)cc4)[nH]n2)n3CC(F)(F)F)CC1 ; CN1CCC(Nc2cccc3c2cc(-c2nnc(CNc4ccc(S(C)(=O)=O)cc4)s2)n3CC(F)(F)F)CC1]

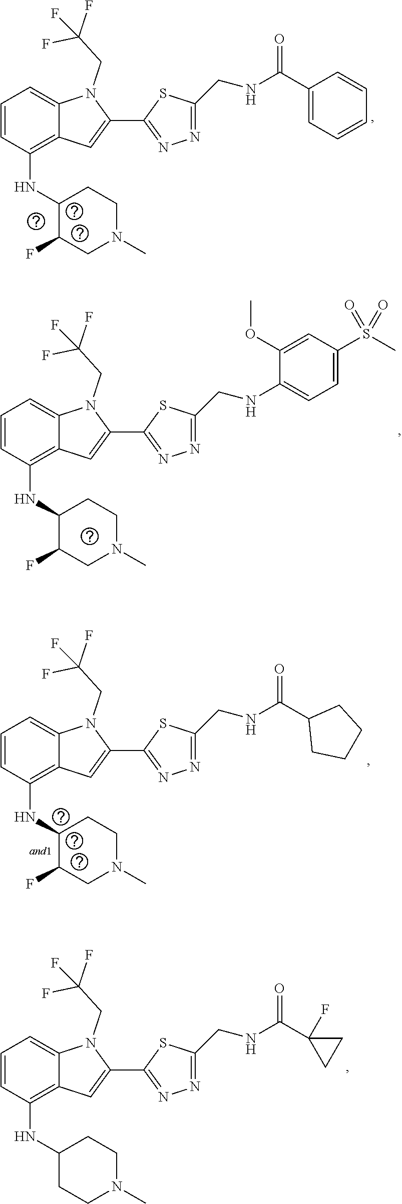 [SMILES: CN1CCC(Nc2cccc3c2cc(-c2nnc(CNC(=O)c4ccccc4)s2)n3CC(F)(F)F)[C@H](F)C1 ; COc1cc(S(C)(=O)=O)ccc1NCc1nnc(-c2cc3c(N[C@H]4CCN(C)C[C@H]4F)cccc3n2CC(F)(F)F)s1 ; CN1CC[C@H](Nc2cccc3c2cc(-c2nnc(CNC(=O)C4CCCC4)s2)n3CC(F)(F)F)[C@H](F)C1 ; CN1CCC(Nc2cccc3c2cc(-c2nnc(CNC(=O)C4(F)CC4)s2)n3CC(F)(F)F)CC1]

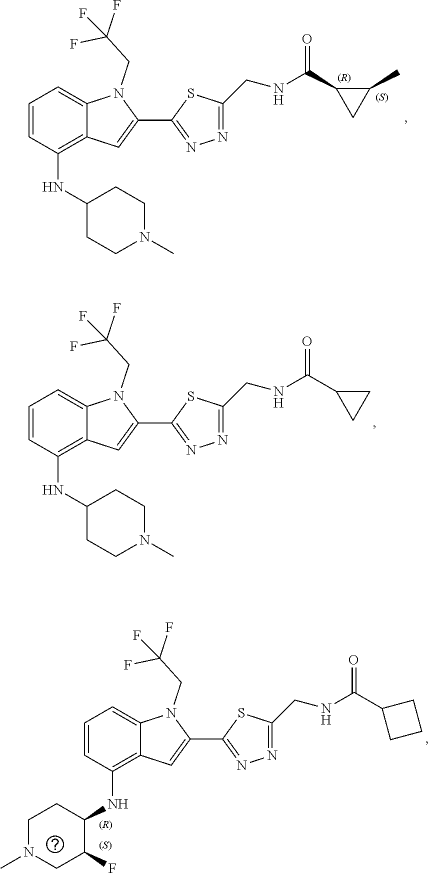

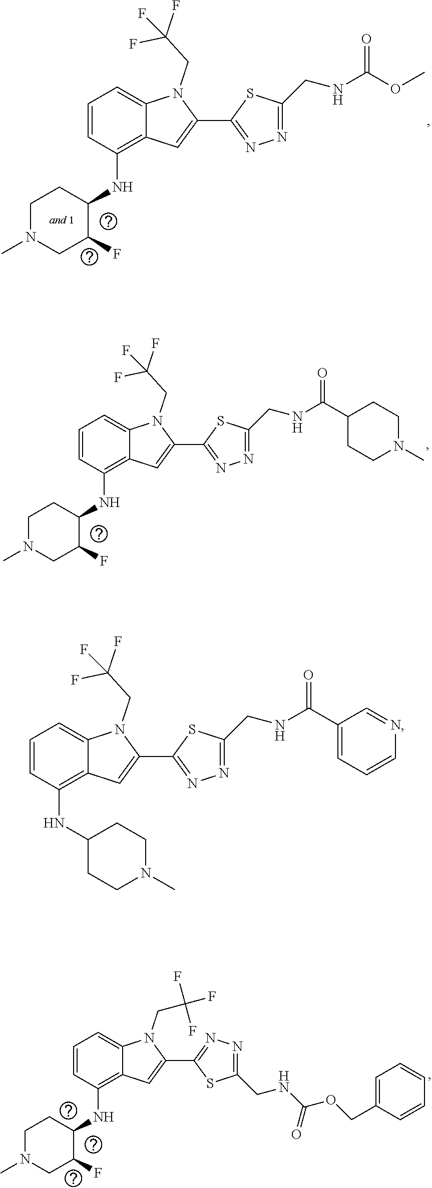

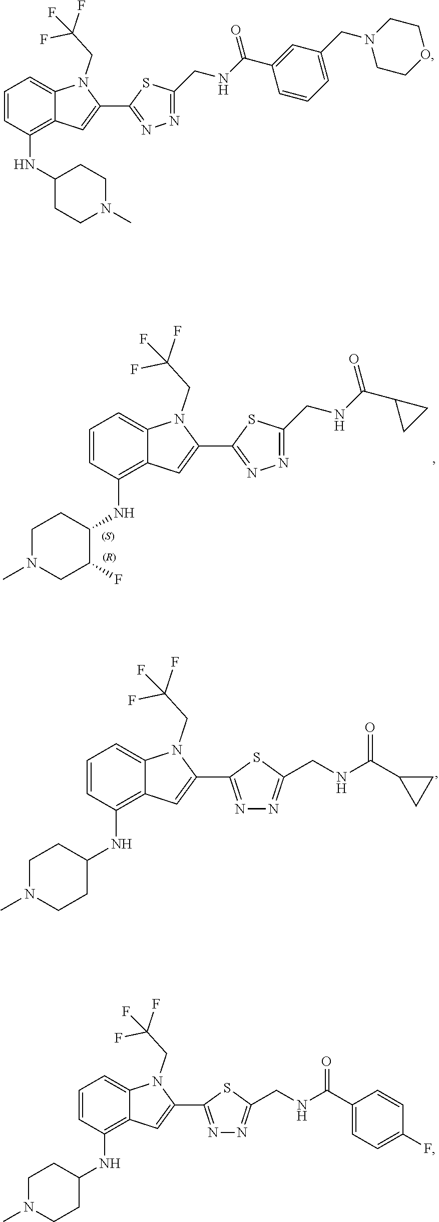

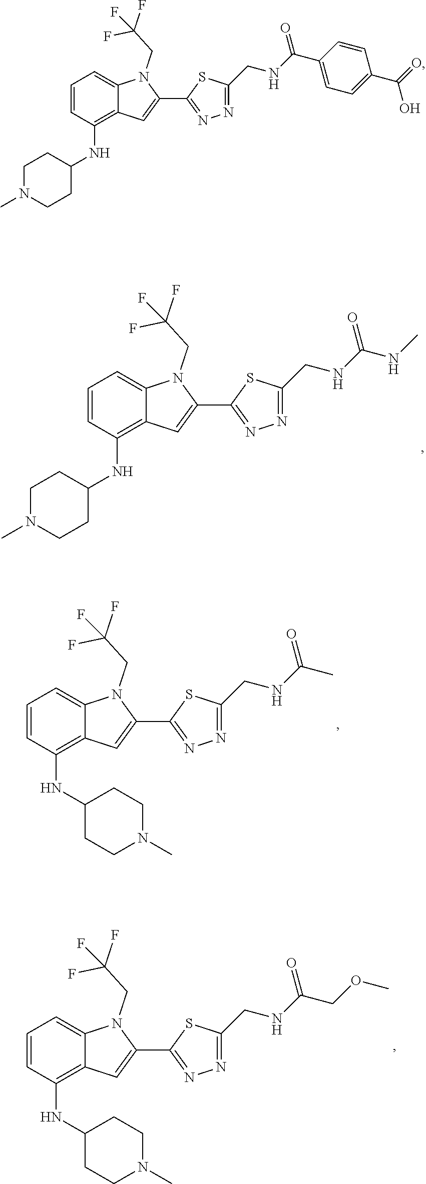

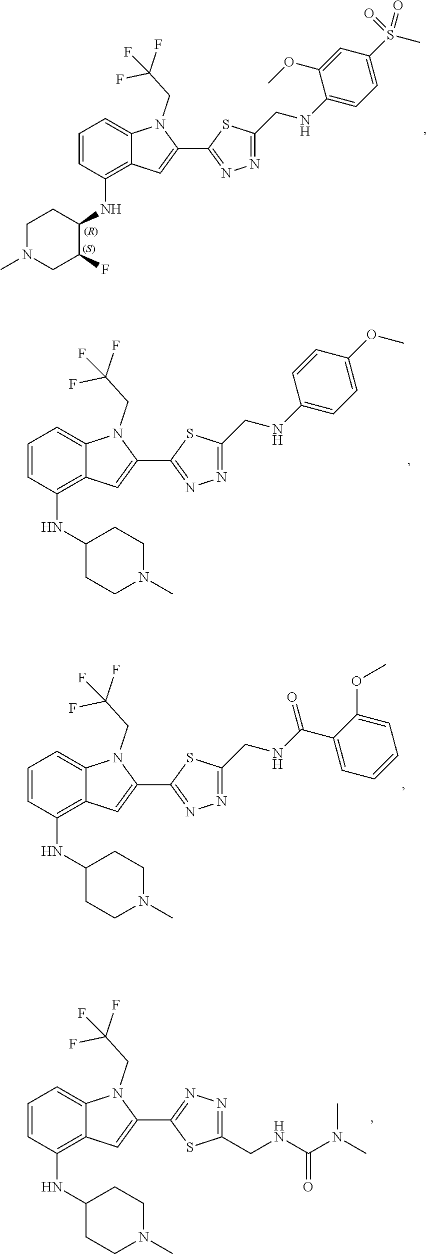

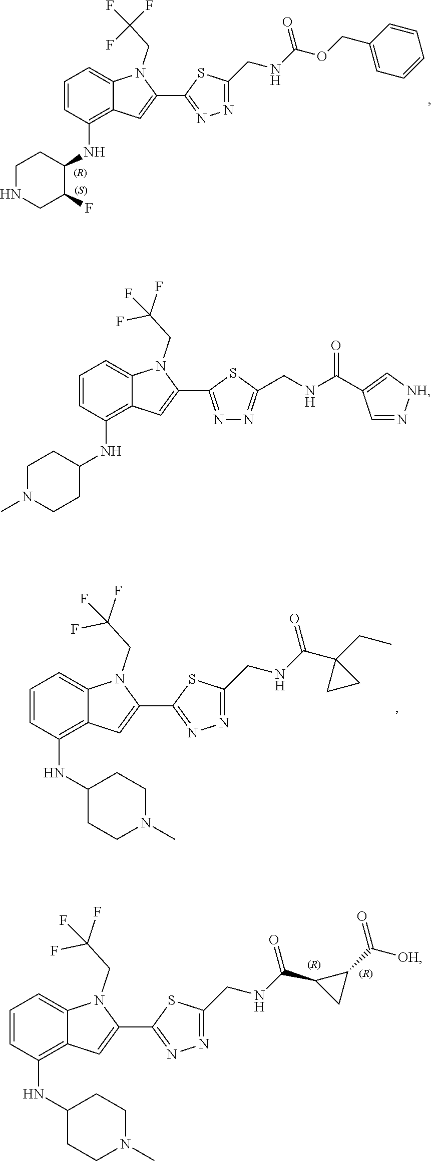

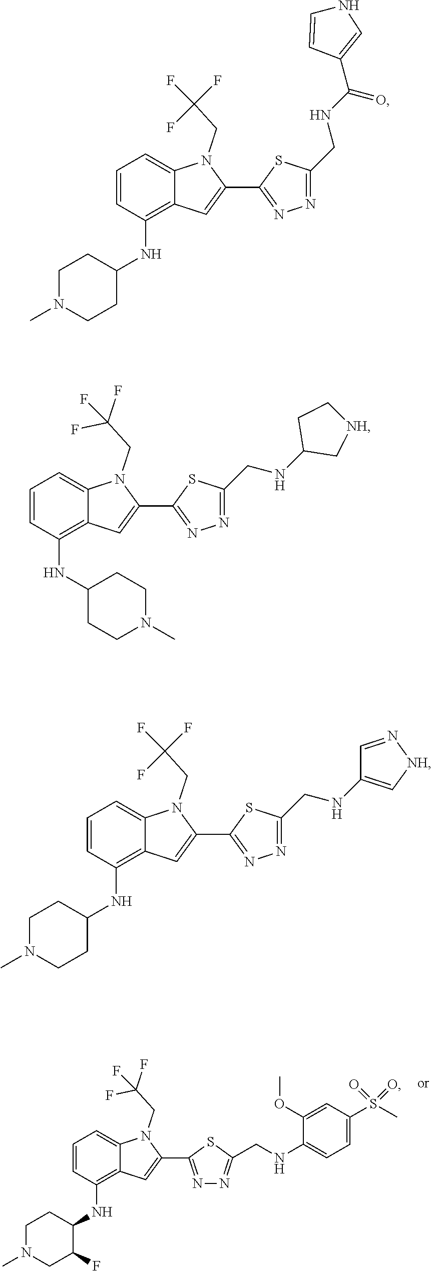

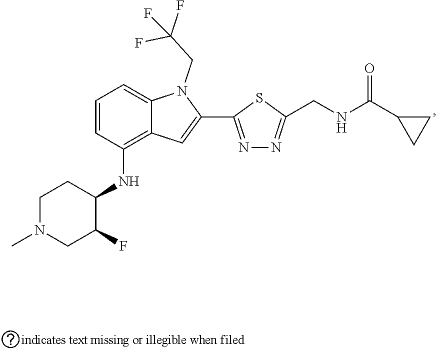 [SMILES: CN1CC[C@@H](Nc2cccc3c2cc(-c2nnc(CNC(=O)C4CC4)s2)n3CC(F)(F)F)[C@@H](F)C1]

or a pharmaceutically-acceptable salt thereof.

Non-limiting examples of compounds of the disclosure include compounds of any of the following formulae:

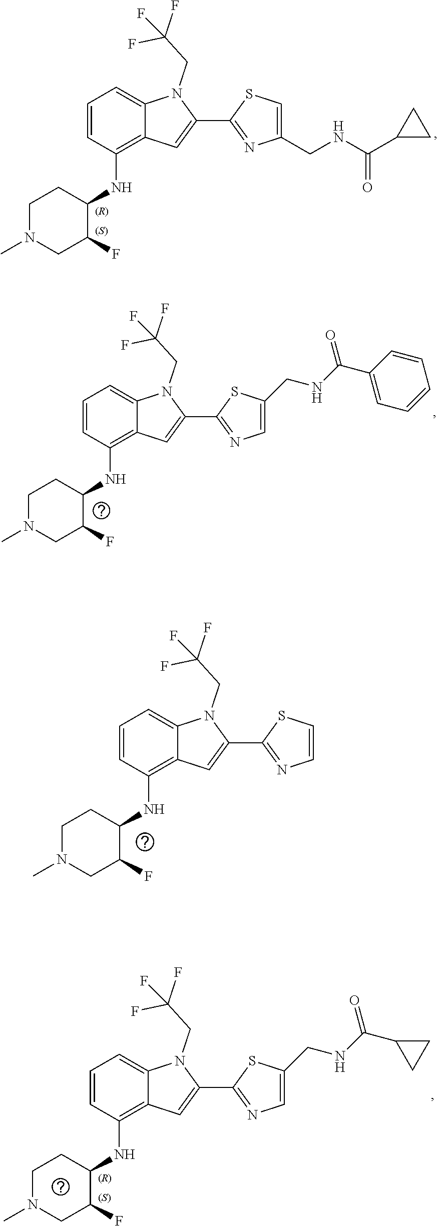

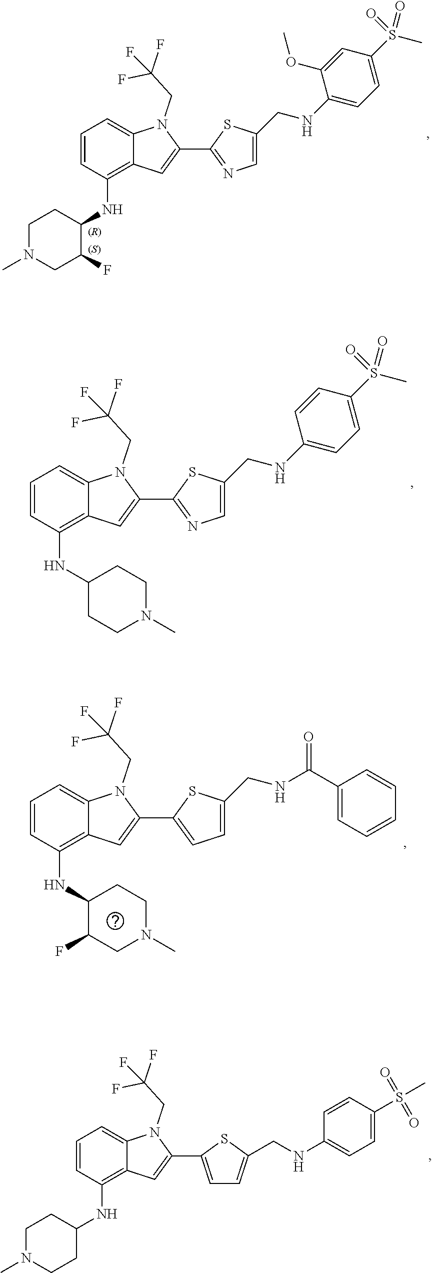

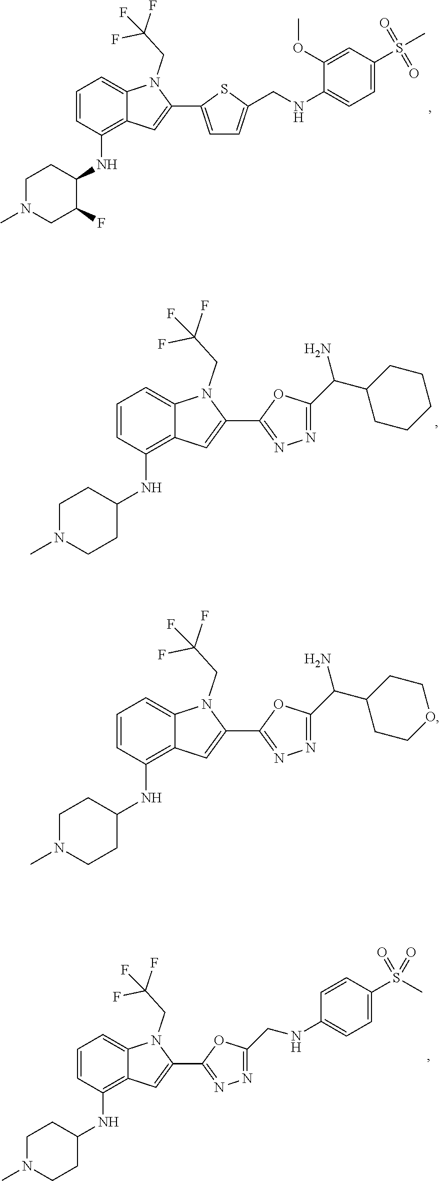

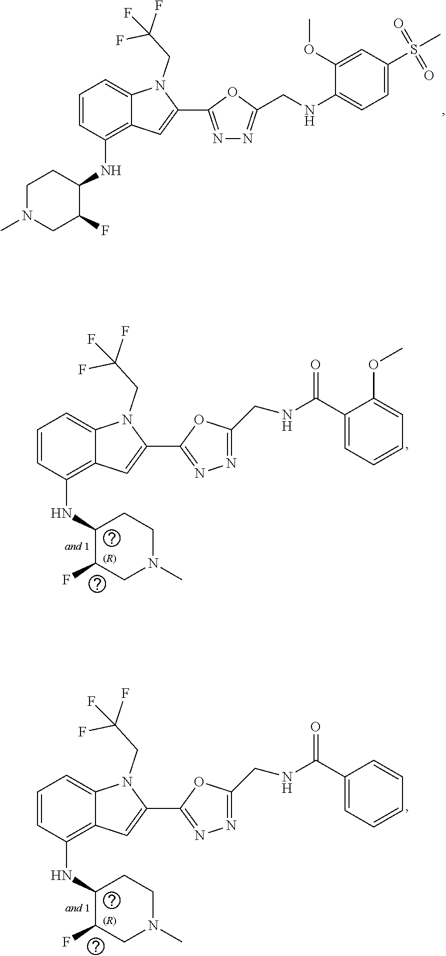

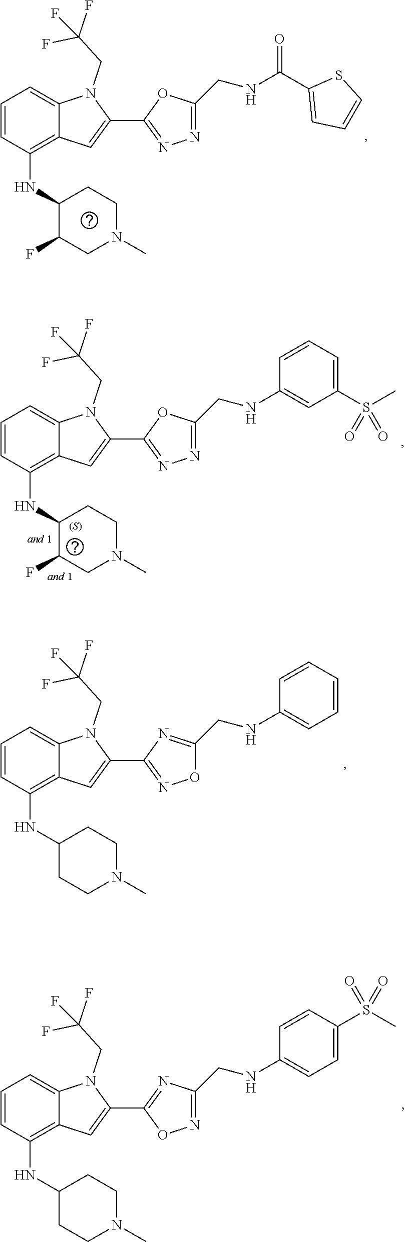

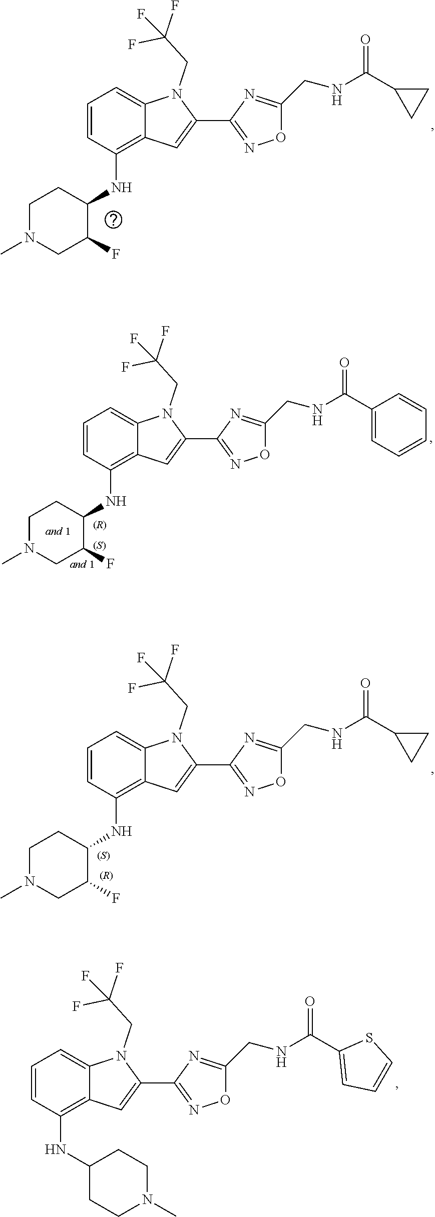

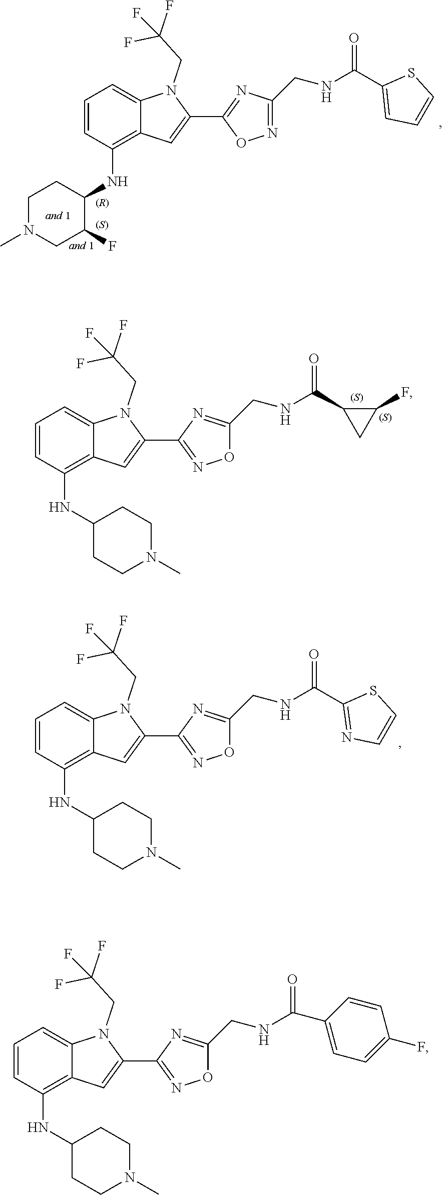

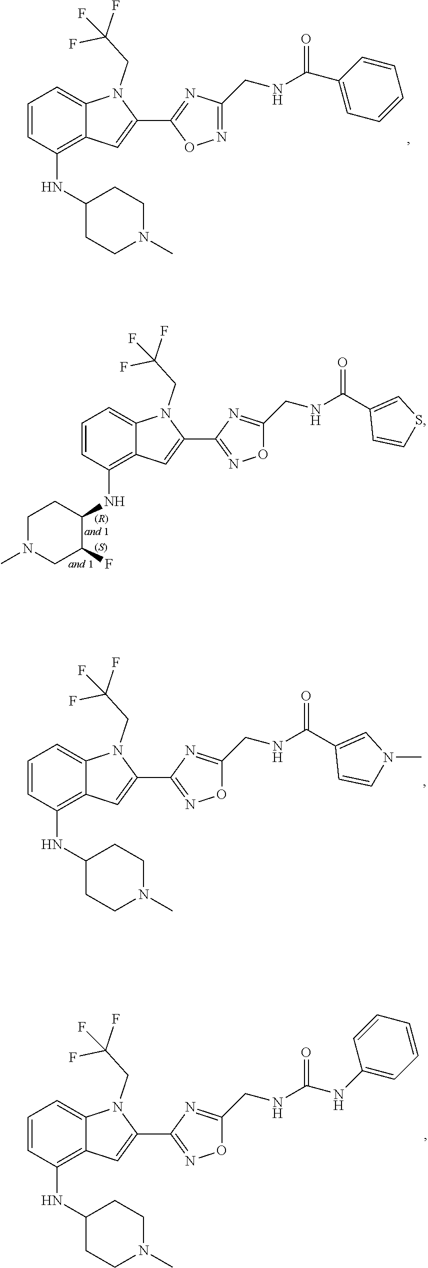

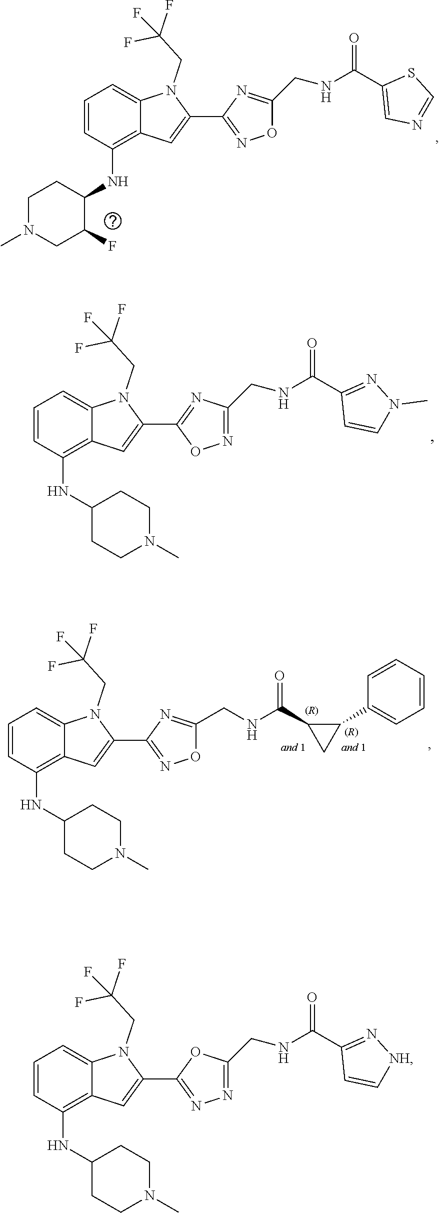

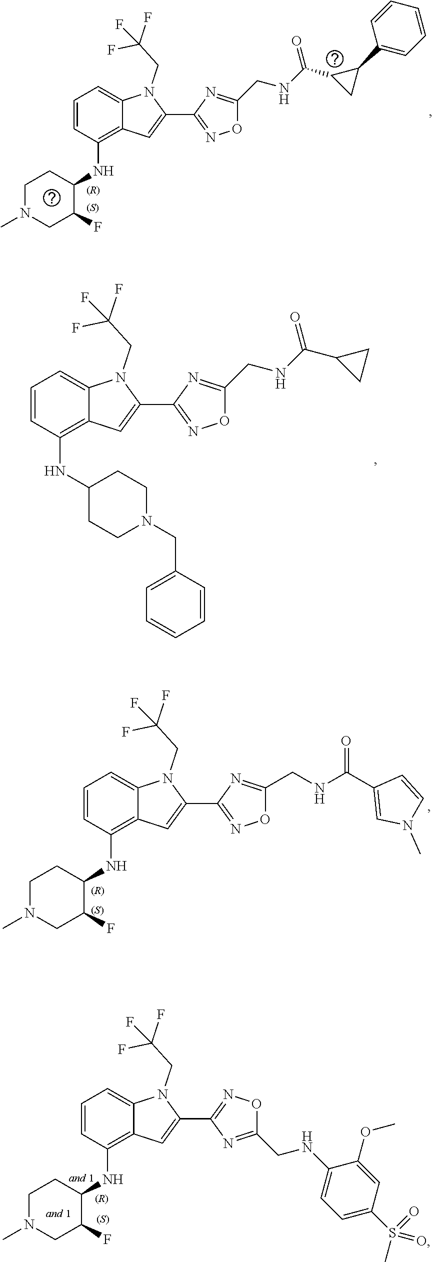

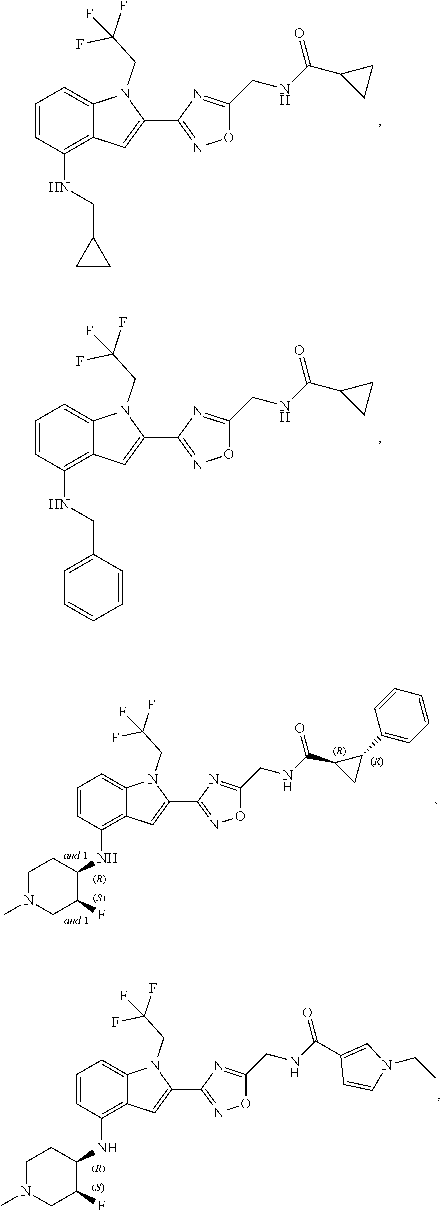

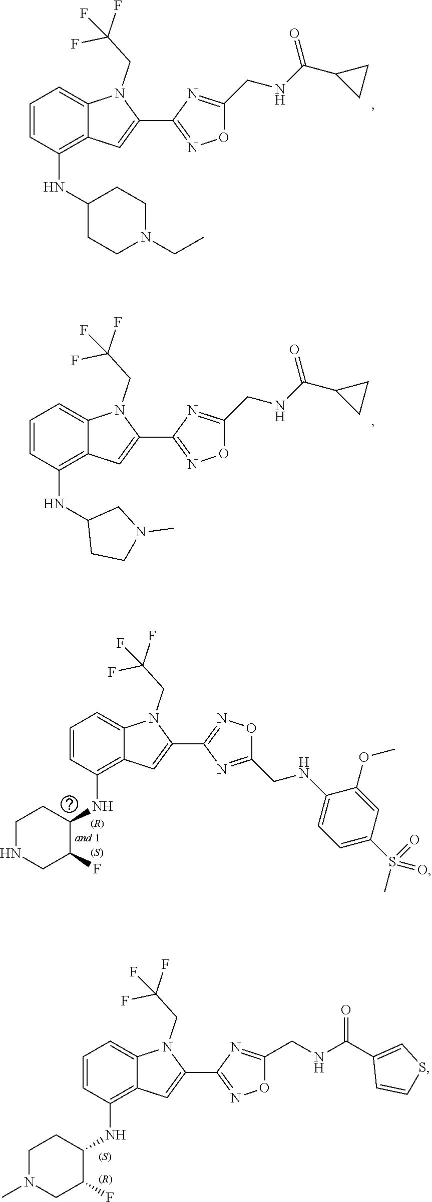

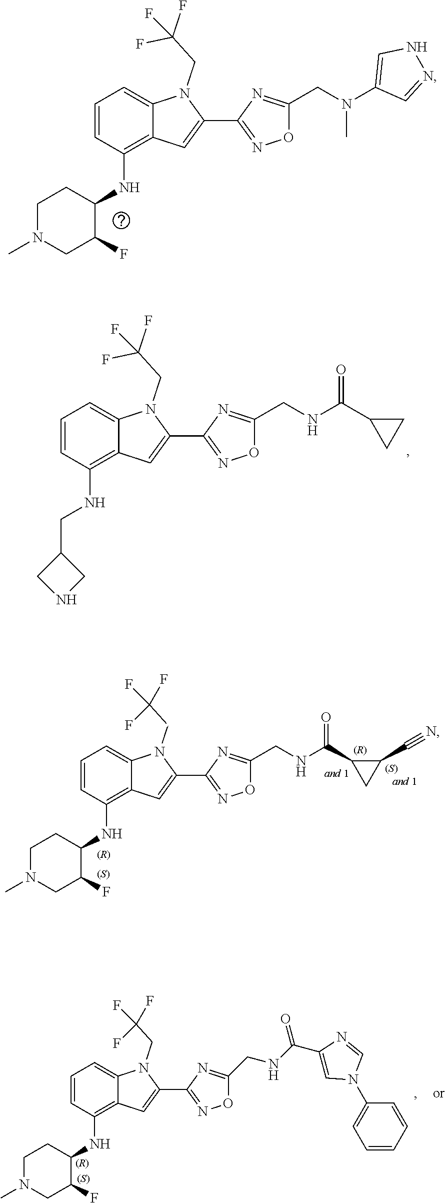

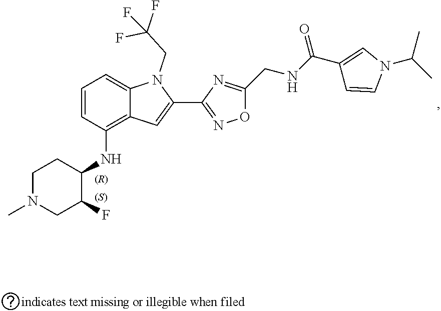

or a pharmaceutically-acceptable salt thereof.

Compounds herein can include all stereoisomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxy ethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically-Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutical Compositions of the Invention.

A pharmaceutical composition of the invention can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds of the invention can be applied topically to the skin, or a body cavity, for example, oral, vaginal, bladder, cranial, spinal, thoracic, or pelvic cavity of a subject. The compounds of the invention can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

### Methods of Use

In some embodiments, compounds of the invention can be used to treat cancer in a subject. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

In some embodiments, the compounds of the invention show non-lethal toxicity.

Disclosed here in are methods of treating cancer, the method comprising administering to a subject in need thereof a therapeutically-effective amount of a compound that binds to a mutant p53 protein and reconforms the mutant p53 protein to a conformation of p53 that exhibits anti-cancer activity, wherein the mutant p53 protein comprises a mutation at Y220C, wherein the compound has a half-maximal inhibitory concentration (IC₅₀) in a cancer cell that has a Y220C mutant p53 protein that is at least about 2-fold lesser than in a cancer cell that does not have any Y220C mutant p53 protein. Also disclosed herein is a method of treating cancer, the method comprising administering to a human in need thereof a therapeutically-effective amount of a compound, wherein the compound binds to a mutant p53 protein and reconforms the mutant p53 protein to a conformation of p53 that exhibits anti-cancer activity, wherein if in a controlled study, the therapeutically-effective amount of the compound is administered to a first subject with a cancer that expresses mutant p53, then a plasma concentration in the first subject of a protein that is a biomarker of wild-type p53 activity when measured from about 8 to about 72 hours after administration of the compound is determined to be at least about 2-fold greater than that determined in a second subject who was not administered the compound, as determined by an enzyme-linked immunosorbent assay. Further disclosed here in is a method of treating cancer, the method comprising: (i) withdrawing a first blood sample from a subject with a cancer that expresses mutant p53; (ii) measuring a first plasma concentration of a protein that is a biomarker of wild-type p53 activity in the first blood sample; (iii) after measuring the first plasma concentration of the protein that is the biomarker of wild-type p53 activity in the first blood sample, administering to the subject a therapeutically-effective amount of a compound that binds to a mutant p53 protein and reconforms the mutant p53 protein to a conformation of p53 that exhibits anti-cancer activity; (iv) withdrawing a second blood sample from the subject after administering the compound; and (v) measuring a second plasma concentration of the protein that is a biomarker of wild-type p53 activity in the second blood sample. Disclosed herein is a method of treating a cancer, the method comprising administering to a subject in need thereof a therapeutically-effective amount of a compound that binds to a mutant p53 protein in the subject and reconforms the mutant p53 protein to a conformation of p53 that exhibits anti-cancer activity within about 2 hours of contacting the cancer with the compound. Also disclosed herein is a method of treating a cancer, the method comprising administering to a subject in need thereof a therapeutically-effective amount of a compound that binds to a mutant p53 protein and reconforms the mutant p53 protein to a conformation of p53 that exhibits anti-cancer activity, wherein the cancer is heterozygous for a p53 Y220C mutation.

Disclosed herein is a method of treating a cancer, the method comprising administering to a subject in need thereof a therapeutically-effective amount of a compound that binds a mutant p53 protein in the subject, wherein binding of the compound to the mutant p53 protein in the subject modulates at least two genes downstream of p53 in the subject, wherein the genes are APAF1, BAX, BBC3, BIRC5, BRCA2, BRCA1, BTG2, CCNB1, CCNE1, CCNG1, CDC25A, CDC25C, CDK1, CDKN1A, CHEK1, CHEK2, E2F1, EGR1, FAS, GADD45A, GAPDH, GDF15, IL6, MDM2, MSH2, p21, PIDD1, PPM1D, PRC1, SESN2, TNFRSF10B, TNFRSF10D, and TP53.

In some embodiments, the conformation of p53 that exhibits anti-cancer activity is a wild type conformation p53 protein. In some embodiments, the biomarker of wild-type p53 activity is MDM2. In some embodiments, the biomarker of wild-type p53 activity is p21.

In some embodiments, the IC₅₀ of the compound is less than about 10 μM, about 9 μM, about 8 μM, about 7 μM, about 6 μM, about 5 μM, about 4 μM, about 3 μM, about 2 μM, about 1 μM, about 0.9 μM, about 0.8 μM, about 0.7 μM, about 0.6 μM, about 0.5 μM, about 0.4 μM, about 0.3 μM, about 0.2 μM, about 0.1 μM, about 0.09 μM, about 0.08 μM, about 0.07 μM, about 0.06 μM, about 0.05 μM, about 0.04 μM, about 0.03 μM, about 0.02 μM, or about 0.01 μM. In some embodiments, the IC₅₀ of the compound is less than about 10 μM. In some embodiments, the IC₅₀ of the compound is less than about 5 μM. In some embodiments, the IC₅₀ of the compound is less than about 1 μM. In some embodiments, the IC₅₀ of the compound is less than about 0.5 μM. In some embodiments, the IC₅₀ of the compound is less than about 0.1 μM. In some embodiments, the IC₅₀ of the compound is determined using an 3-(4,5-Dimethylthiazol-2-yl)-2,5 diphenyltetrazolium bromide (MTT) assay.

In some embodiments, the methods of the disclosure further comprise administering a therapeutically-effective amount of a therapeutic agent. In some embodiments, the therapeutic is an immune checkpoint inhibitor, for example, an anti-PD-1 agent or anti-PD-L1 agent. In some embodiments, the anti-PD-1 agent is nivolumab. In some embodiments, the anti-PD-1 agent is pembrolizumab. In some embodiments, the anti-PD-1 agent is cemiplimab. In some embodiments, the anti-PD-L1 agent is atezolizumab. In some embodiments, the anti-PD-L1 agent is avelumab. In some embodiments, the anti-PD-L1 agent is durvalumab.

In some embodiments, the compound increases a stability of the mutant p53 protein. In some embodiments, the cancer expresses a mutant p53 protein. In some embodiments, the mutant p53 protein has a mutation at amino acid 220. In some embodiments, the mutant p53 protein is p53 Y220C. In some embodiments, the compound selectively binds the mutant p53 protein as compared to a wild type p53. In some embodiments, the cancer is ovarian cancer. In some embodiments, the cancer is breast cancer. In some embodiments, the cancer is lung cancer. In some embodiments, the subject is human.

In some embodiments, the administering of the compound is oral. In some embodiments, the administering of the compound is subcutaneous. In some embodiments, the administering of the compound is topical. In some embodiments, the therapeutically-effective amount of the compound is from about 1 mg/kg to about 500 mg/kg. In some embodiments, the therapeutically-effective amount of the compound is from about 100 mg to about 5000 mg. In some embodiments, the therapeutically-effective amount of the compound is from about 500 mg to about 2000 mg. In some embodiments, the therapeutically-effective amount of the compound is about 250 mg, about 500 mg, about 750 mg, about 1000 mg, about 1250 mg, about 1500 mg, about 1750 mg, about 2000 mg, about 2250 mg, or about 2500 mg. In some embodiments, the therapeutically-effective amount of the compound is about 150 mg. In some embodiments, the therapeutically-effective amount of the compound is about 300 mg. In some embodiments, the therapeutically-effective amount of the compound is about 500 mg. In some embodiments, the therapeutically-effective amount of the compound is about 600 mg. In some embodiments, the therapeutically-effective amount of the compound is about 1200 mg. In some embodiments, the therapeutically-effective amount of the compound is about 1500 mg. In some embodiments, the therapeutically-effective amount of the compound is about 2000 mg.

In some embodiments, the plasma concentration in the first subject is measured about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration of the compound. In some embodiments, the plasma concentration in the first subject is measured about 8 hours after administration of the compound. In some embodiments, the plasma concentration in the first subject is measured about 12 hours after administration of the compound. In some embodiments, the plasma concentration in the first subject is measured about 24 hours after administration of the compound.

In some embodiments, the plasma concentration of the first subject is at least about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, or about 40-fold greater than that determined in the second subject. In some embodiments, the plasma concentration of the first subject is at least about 5-fold greater than that determined in the second subject. In some embodiments, the plasma concentration of the first subject is at least about 8-fold greater than that determined in the second subject. In some embodiments, the plasma concentration of the first subject is at least about 10-fold greater than that determined in the second subject. In some embodiments, the plasma concentration of the first subject is at least about 15-fold greater than that determined in the second subject. In some embodiments, the plasma concentration of the first subject is at least about 20-fold greater than that determined in the second subject. In some embodiments, the plasma concentration of the first subject is at least about 25-fold greater than that determined in the second subject. In some embodiments, the plasma concentration of the first subject is at least about 40-fold greater than that determined in the second subject.

In some embodiments, the second plasma concentration of the protein is equal to the first plasma concentration of the protein. In some embodiments, the methods further comprise administering a second therapeutically-effective amount of the compound. In some embodiments, the second plasma concentration of the protein is lower than the first plasma concentration of the protein. In some embodiments, the methods further comprise administering a second therapeutically-effective amount of the compound.

In some embodiments, the biomarker is MDM2. In some embodiments, the plasma concentration of MDM2 in a subject administered with a compound of the disclosure is about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, or about 40-fold greater than the plasma concentration of MDM2 in a subject that is not administered with the compound. In some embodiments, the plasma concentration of MDM2 in a subject administered with a compound of the disclosure is about 5-fold greater than the plasma concentration of MDM2 in a subject that is not administered with the compound. In some embodiments, the plasma concentration of MDM2 in a subject administered with a compound of the disclosure is about 8-fold greater than the plasma concentration of MDM2 in a subject that is not administered with the compound. In some embodiments, the plasma concentration of MDM2 in a subject administered with a compound of the disclosure is about 20-fold greater than the plasma concentration of MDM2 in a subject that is not administered with the compound. In some embodiments, the plasma concentration of MDM2 in a subject administered with a compound of the disclosure is about 40-fold greater than the plasma concentration of MDM2 in a subject that is not administered with the compound.

In some embodiments, the biomarker is p21. In some embodiments, the plasma concentration of p21 in a subject administered with a compound of the disclosure is about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, or about 40-fold greater than the plasma concentration of p21 in a subject that is not administered with the compound. In some embodiments, the plasma concentration of p21 in a subject administered with a compound of the disclosure is about 5-fold greater than the plasma concentration of p21 in a subject that is not administered with the compound. In some embodiments, the plasma concentration of p21 in a subject administered with a compound of the disclosure is about 8-fold greater than the plasma concentration of p21 in a subject that is not administered with the compound. In some embodiments, the plasma concentration of p21 in a subject administered with a compound of the disclosure is about 20-fold greater than the plasma concentration of p21 in a subject that is not administered with the compound. In some embodiments, the plasma concentration of p21 in a subject administered with a compound of the disclosure is about 40-fold greater than the plasma concentration of p21 in a subject that is not administered with the compound.

In some embodiments, the cancer is ovarian cancer. In some embodiments, the cancer is breast cancer. In some embodiments, the cancer is lung cancer.

In some embodiments, administering a compound to a subject can decrease mutant p53 levels in the subject by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, administering a compound to a subject can decrease mutant p53 levels in the subject by about 50%. In some embodiments, administering a compound to a subject can decrease mutant p53 levels in the subject by about 60%. In some embodiments, administering a compound to a subject can decrease mutant p53 levels in the subject by about 80%. In some embodiments, administering a compound to a subject can decrease mutant p53 levels in the subject by about 90%.

In some embodiments, a decrease in mutant p53 levels or increase in plasma concentration of a biomarker indicative of wild type p53 is sustained for about 4 hours, about 6 hours, about 8 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, or about 36 hours. In some embodiments, a decrease in mutant p53 levels or increase in plasma concentration of a biomarker indicative of wild type p53 is sustained for about 4 hours. In some embodiments, a decrease in mutant p53 levels or increase in plasma concentration of a biomarker indicative of wild type p53 is sustained for about 8 hours. In some embodiments, a decrease in mutant p53 levels or increase in plasma concentration of a biomarker indicative of wild type p53 is sustained for about 12 hours. In some embodiments, a decrease in mutant p53 levels or increase in plasma concentration of a biomarker indicative of wild type p53 is sustained for about 24 hours.

In some embodiments, the compounds of the disclosure modulate two genes. In some embodiments, the compounds of the disclosure modulate three genes. In some embodiments, the compounds of the disclosure modulate four genes. In some embodiments, the compounds of the disclosure modulate five genes. In some embodiments, the at least two genes comprises p21. In some embodiments, the at least two genes comprises MDM2. In some embodiments, the at least two genes comprises GDF15. In some embodiments, the at least two genes comprises GAPDH.

The methods of the disclosure can administer a compound or structure comprising a substituted heterocyclyl group. In some embodiments, the structure comprises a heterocyclyl group comprising a halo substituent. In some embodiments, the structure comprises an indole group. In some embodiments, the indole group comprises a propargyl substituent at a 2-position of the indole group. In some embodiments, the propargyl substituent is attached to the indole group via an sp carbon atom of the propargyl substituent. In some embodiments, the propargyl substituent is attached to a nitrogen atom of an aniline group via a methylene group of the propargyl substituent. In some embodiments, the indole group comprises an amino substituent at a 4-position of the indole group. In some embodiments, the amino substituent is attached to the heterocyclyl group.

In some embodiments, the compound is of the formula:

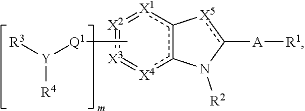

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, A is alkylene, alkenylene, or alkynylene, each of which is substituted or unsubstituted. In some embodiments, A is aryl, heteroaryl, or heterocyclyl, each of which is substituted or unsubstituted. In some embodiments, the compound is of the formula:

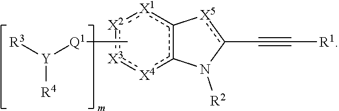

In some embodiments, Q¹ is C₁-alkylene. In some embodiments, Q¹ is a bond. In some embodiments, m is 1. In some embodiments, m is 2. In some embodiments, Y is N. In some embodiments, Y is O. In some embodiments, each R³ and R⁴ is independently alkyl, alkylene, alkenyl, alkenylene, alkynyl, aryl, heteroaryl, or heterocyclyl, each of which is independently substituted or unsubstituted; or hydrogen. In some embodiments, R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted. In some embodiments, R³ is H; and R⁴ is aryl, heteroaryl, or heterocyclyl, each of which is independently substituted or unsubstituted. R¹³ is hydrogen.

In some embodiments, the compound is of the formula:

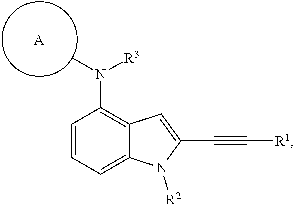

wherein ring A is a cyclic group that is substituted or unsubstituted. In some embodiments, R² is substituted or unsubstituted alkyl. In some embodiments, R² is methyl, ethyl, propyl, iso-propyl, butyl, or tert-butyl, each of which is substituted or unsubstituted. In some embodiments, R² is substituted ethyl. In some embodiments, R² is trifluoroethyl.

In some embodiments, the compound is of the formula

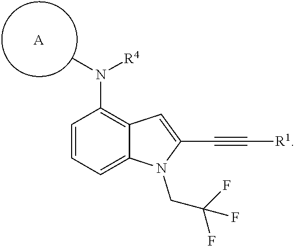

In some embodiments, ring A is aryl, heteroaryl, or heterocyclyl, each of which is substituted or unsubstituted. In some embodiments, ring A is substituted aryl. In some embodiments, ring A is substituted heteroaryl. In some embodiments, ring A is substituted heterocyclyl.

In some embodiments, R¹ is alkyl, alkenyl, —C(O)R¹⁶, —C(O)OR¹⁶, or —C(O)NR¹⁶R¹⁷, each of which is unsubstituted or substituted. In some embodiments, R¹ is substituted alkyl. In some embodiments, R¹ is alkyl substituted with NR¹⁶R¹⁷. In some embodiments, the compound is of the formula:

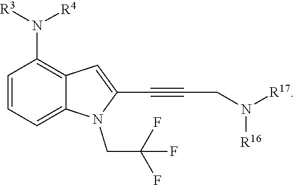

In some embodiments, each R¹⁶ and R¹⁷ is independently alkyl, alkenyl, aryl, heteroaryl, heterocyclyl, each of which is independently substituted or unsubstituted; or hydrogen. In some embodiments, R¹⁶ is hydrogen or alkyl. In some embodiments, R¹⁷ is aryl, heteroaryl, or heterocyclyl, each of which is independently substituted or unsubstituted. In some embodiments, R¹⁷ is substituted aryl. In some embodiments, R¹⁷ is substituted phenyl. In some embodiments, R¹⁷ is phenyl substituted with a sulfoxide group, carboxyl group, amide group, amino group, alkyl, alkoxy, hydroxy, halo, cyano, or heterocyclyl, each of which is independently substituted or unsubstituted. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is phenyl substituted with an amide group.

In some embodiments, the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide. In some embodiments, the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine. In some embodiments, the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide. In some embodiments, the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide. In some embodiments, the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide. In some embodiments, the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide. In some embodiments, the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide. In some embodiments, the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide. In some embodiments, the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

Pharmacokinetic and pharmacodynamic data can be obtained by various experimental techniques. Appropriate pharmacokinetic and pharmacodynamic profile components describing a particular composition can vary due to variations in drug metabolism in human subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 15 subjects, 20 subjects, 25 subjects, 30 subjects, 35 subjects, or more. The mean is determined, for example, by calculating the average of all subject's measurements for each parameter measured. A dose can be modulated to achieve a desired pharmacokinetic or pharmacodynamics profile, such as a desired or effective blood profile, as described herein.

The pharmacodynamic parameters can be any parameters suitable for describing compositions of the invention. For example, the pharmacodynamic profile can be obtained at a time after dosing of, for example, about zero minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about zero hours, about 0.5 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, or about 24 hours.

The pharmacokinetic parameters can be any parameters suitable for describing a compound. The Cₘₐₓ can be, for example, not less than about 1 ng/mL; not less than about 5 ng/mL; not less than about 10 ng/mL; not less than about 15 ng/mL; not less than about 20 ng/mL; not less than about 25 ng/mL; not less than about 50 ng/mL; not less than about 75 ng/mL; not less than about 100 ng/mL; not less than about 200 ng/mL; not less than about 300 ng/mL; not less than about 400 ng/mL; not less than about 500 ng/mL; not less than about 600 ng/mL; not less than about 700 ng/mL; not less than about 800 ng/mL; not less than about 900 ng/mL; not less than about 1000 ng/mL; not less than about 1250 ng/mL; not less than about 1500 ng/mL; not less than about 1750 ng/mL; not less than about 2000 ng/mL; or any other Cₘₐₓ appropriate for describing a pharmacokinetic profile of a compound described herein. The Cₘₐₓ can be, for example, about 1 ng/mL to about 5,000 ng/mL; about 1 ng/mL to about 4,500 ng/mL; about 1 ng/mL to about 4,000 ng/mL; about 1 ng/mL to about 3,500 ng/mL; about 1 ng/mL to about 3,000 ng/mL; about 1 ng/mL to about 2,500 ng/mL; about 1 ng/mL to about 2,000 ng/mL; about 1 ng/mL to about 1,500 ng/mL; about 1 ng/mL to about 1,000 ng/mL; about 1 ng/mL to about 900 ng/mL; about 1 ng/mL to about 800 ng/mL; about 1 ng/mL to about 700 ng/mL; about 1 ng/mL to about 600 ng/mL; about 1 ng/mL to about 500 ng/mL; about 1 ng/mL to about 450 ng/mL; about 1 ng/mL to about 400 ng/mL; about 1 ng/mL to about 350 ng/mL; about 1 ng/mL to about 300 ng/mL; about 1 ng/mL to about 250 ng/mL; about 1 ng/mL to about 200 ng/mL; about 1 ng/mL to about 150 ng/mL; about 1 ng/mL to about 125 ng/mL; about 1 ng/mL to about 100 ng/mL; about 1 ng/mL to about 90 ng/mL; about 1 ng/mL to about 80 ng/mL; about 1 ng/mL to about 70 ng/mL; about 1 ng/mL to about 60 ng/mL; about 1 ng/mL to about 50 ng/mL; about 1 ng/mL to about 40 ng/mL; about 1 ng/mL to about 30 ng/mL; about 1 ng/mL to about 20 ng/mL; about 1 ng/mL to about 10 ng/mL; about 1 ng/mL to about 5 ng/mL; about 10 ng/mL to about 4,000 ng/mL; about 10 ng/mL to about 3,000 ng/mL; about 10 ng/mL to about 2,000 ng/mL; about 10 ng/mL to about 1,500 ng/mL; about 10 ng/mL to about 1,000 ng/mL; about 10 ng/mL to about 900 ng/mL; about 10 ng/mL to about 800 ng/mL; about 10 ng/mL to about 700 ng/mL; about 10 ng/mL to about 600 ng/mL; about 10 ng/mL to about 500 ng/mL; about 10 ng/mL to about 400 ng/mL; about 10 ng/mL to about 300 ng/mL; about 10 ng/mL to about 200 ng/mL; about 10 ng/mL to about 100 ng/mL; about 10 ng/mL to about 50 ng/mL; about 25 ng/mL to about 500 ng/mL; about 25 ng/mL to about 100 ng/mL; about 50 ng/mL to about 500 ng/mL; about 50 ng/mL to about 100 ng/mL; about 100 ng/mL to about 500 ng/mL; about 100 ng/mL to about 400 ng/mL; about 100 ng/mL to about 300 ng/mL; or about 100 ng/mL to about 200 ng/mL.

The Tₘₐₓ of a compound described herein can be, for example, not greater than about 0.5 hours, not greater than about 1 hours, not greater than about 1.5 hours, not greater than about 2 hours, not greater than about 2.5 hours, not greater than about 3 hours, not greater than about 3.5 hours, not greater than about 4 hours, not greater than about 4.5 hours, not greater than about 5 hours, or any other Tₘₐₓ appropriate for describing a pharmacokinetic profile of a compound described herein. The Tₘₐₓ can be, for example, about 0.1 hours to about 24 hours; about 0.1 hours to about 0.5 hours; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; or about 23.5 hours to about 24 hours. In some embodiments, the Tₘₐₓ of a compound of the disclosure is about 2 hours. In some embodiments, the Tₘₐₓ of a compound of the disclosure is about 4 hours. In some embodiments, the Tₘₐₓ of a compound of the disclosure is about 6 hours. In some embodiments, the Tₘₐₓ of a compound of the disclosure is about 8 hours.

The AUC_{(0-inf)} or AUC₍ₗₐₛₜ₎ of a compound described herein can be, for example, not less than about 1 ng·hr/mL, not less than about 5 ng·hr/mL, not less than about 10 ng·hr/mL, not less than about 20 ng·hr/mL, not less than about 30 ng·hr/mL, not less than about 40 ng·hr/mL, not less than about 50 ng·hr/mL, not less than about 100 ng·hr/mL, not less than about 150 ng·hr/mL, not less than about 200 ng·hr/mL, not less than about 250 ng·hr/mL, not less than about 300 ng·hr/mL, not less than about 350 ng·hr/mL, not less than about 400 ng·hr/mL, not less than about 450 ng·hr/mL, not less than about 500 ng·hr/mL, not less than about 600 ng·hr/mL, not less than about 700 ng·hr/mL, not less than about 800 ng·hr/mL, not less than about 900 ng·hr/mL, not less than about 1000 ng·hr/mL, not less than about 1250 ng·hr/mL, not less than about 1500 ng·hr/mL, not less than about 1750 ng·hr/mL, not less than about 2000 ng·hr/mL, not less than about 2500 ng·hr/mL, not less than about 3000 ng·hr/mL, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, not less than about 10,000 ng·hr/mL, or any other AUC_{(0-inf)} or AUC₍ₗₐₛₜ₎ appropriate for describing a pharmacokinetic profile of a compound described herein. In some embodiments, the AUC_{(0-inf)} or AUC₍ₗₐₛₜ₎ of a compound described herein can be, for example, not less than about 10,000 ng·hr/mL, not less than about 11,000 ng·hr/mL, not less than about 12,000 ng·hr/mL, not less than about 13,000 ng·hr/mL, not less than about 14,000 ng·hr/mL, not less than about 15,000 ng·hr/mL, not less than about 16,000 ng·hr/mL, not less than about 17,000 ng·hr/mL, not less than about 18,000 ng·hr/mL, not less than about 19,000 ng·hr/mL, not less than about 20,000 ng·hr/mL, not less than about 21,000 ng·hr/mL, not less than about 22,000 ng·hr/mL, not less than about 23,000 ng·hr/mL, not less than about 24,000 ng·hr/mL, or not less than about 25,000 ng·hr/mL.

The AUC_{(0-inf)} or AUC₍ₗₐₛₜ₎ of a compound can be, for example, about 1 ng·hr/mL to about 10,000 ng·hr/mL; about 1 ng·hr/mL to about 10 ng·hr/mL; about 10 ng·hr/mL to about 25 ng·hr/mL; about 25 ng·hr/mL to about 50 ng·hr/mL; about 50 ng·hr/mL to about 100 ng·hr/mL; about 100 ng·hr/mL to about 200 ng·hr/mL; about 200 ng·hr/mL to about 300 ng·hr/mL; about 300 ng·hr/mL to about 400 ng·hr/mL; about 400 ng·hr/mL to about 500 ng·hr/mL; about 500 ng·hr/mL to about 600 ng·hr/mL; about 600 ng·hr/mL to about 700 ng·hr/mL; about 700 ng·hr/mL to about 800 ng·hr/mL; about 800 ng·hr/mL to about 900 ng·hr/mL; about 900 ng·hr/mL to about 1,000 ng·hr/mL; about 1,000 ng·hr/mL to about 1,250 ng·hr/mL; about 1,250 ng·hr/mL to about 1,500 ng·hr/mL; about 1,500 ng·hr/mL to about 1,750 ng·hr/mL; about 1,750 ng·hr/mL to about 2,000 ng·hr/mL; about 2,000 ng·hr/mL to about 2,500 ng·hr/mL; about 2,500 ng·hr/mL to about 3,000 ng·hr/mL; about 3,000 ng·hr/mL to about 3,500 ng·hr/mL; about 3,500 ng·hr/mL to about 4,000 ng·hr/mL; about 4,000 ng·hr/mL to about 4,500 ng·hr/mL; about 4,500 ng·hr/mL to about 5,000 ng·hr/mL; about 5,000 ng·hr/mL to about 5,500 ng·hr/mL; about 5,500 ng·hr/mL to about 6,000 ng·hr/mL; about 6,000 ng·hr/mL to about 6,500 ng·hr/mL; about 6,500 ng·hr/mL to about 7,000 ng·hr/mL; about 7,000 ng·hr/mL to about 7,500 ng·hr/mL; about 7,500 ng·hr/mL to about 8,000 ng·hr/mL; about 8,000 ng·hr/mL to about 8,500 ng·hr/mL; about 8,500 ng·hr/mL to about 9,000 ng·hr/mL; about 9,000 ng·hr/mL to about 9,500 ng·hr/mL; or about 9,500 ng·hr/mL to about 10,000 ng·hr/mL. In some embodiments, the AUC_{(0-inf)} or AUC₍ₗₐₛₜ₎ of a compound described herein can be, for example, about 10,000 ng·hr/mL, about 11,000 ng·hr/mL, about 12,000 ng·hr/mL, about 13,000 ng·hr/mL, about 14,000 ng·hr/mL, about 15,000 ng·hr/mL, about 16,000 ng·hr/mL, about 17,000 ng·hr/mL, about 18,000 ng·hr/mL, about 19,000 ng·hr/mL, about 20,000 ng·hr/mL, about 21,000 ng·hr/mL, about 22,000 ng·hr/mL, about 23,000 ng·hr/mL, about 24,000 ng·hr/mL, or about 25,000 ng·hr/mL.

The plasma concentration of a compound described herein can be, for example, not less than about 1 ng/mL, not less than about 5 ng/mL, not less than about 10 ng/mL, not less than about 15 ng/mL, not less than about 20 ng/mL, not less than about 25 ng/mL, not less than about 50 ng/mL, not less than about 75 ng/mL, not less than about 100 ng/mL, not less than about 150 ng/mL, not less than about 200 ng/mL, not less than about 300 ng/mL, not less than about 400 ng/mL, not less than about 500 ng/mL, not less than about 600 ng/mL, not less than about 700 ng/mL, not less than about 800 ng/mL, not less than about 900 ng/mL, not less than about 1000 ng/mL, not less than about 1200 ng/mL, or any other plasma concentration of a compound described herein. The plasma concentration can be, for example, about 1 ng/mL to about 2,000 ng/mL; about 1 ng/mL to about 5 ng/mL; about 5 ng/mL to about 10 ng/mL; about 10 ng/mL to about 25 ng/mL; about 25 ng/mL to about 50 ng/mL; about 50 ng/mL to about 75 ng/mL; about 75 ng/mL to about 100 ng/mL; about 100 ng/mL to about 150 ng/mL; about 150 ng/mL to about 200 ng/mL; about 200 ng/mL to about 250 ng/mL; about 250 ng/mL to about 300 ng/mL; about 300 ng/mL to about 350 ng/mL; about 350 ng/mL to about 400 ng/mL; about 400 ng/mL to about 450 ng/mL; about 450 ng/mL to about 500 ng/mL; about 500 ng/mL to about 600 ng/mL; about 600 ng/mL to about 700 ng/mL; about 700 ng/mL to about 800 ng/mL; about 800 ng/mL to about 900 ng/mL; about 900 ng/mL to about 1,000 ng/mL; about 1,000 ng/mL to about 1,100 ng/mL; about 1,100 ng/mL to about 1,200 ng/mL; about 1,200 ng/mL to about 1,300 ng/mL; about 1,300 ng/mL to about 1,400 ng/mL; about 1,400 ng/mL to about 1,500 ng/mL; about 1,500 ng/mL to about 1,600 ng/mL; about 1,600 ng/mL to about 1,700 ng/mL; about 1,700 ng/mL to about 1,800 ng/mL; about 1,800 ng/mL to about 1,900 ng/mL; or about 1,900 ng/mL to about 2,000 ng/mL.

In some embodiments, the plasma concentration can be about 2,500 ng/mL, about 3,000 ng/mL, about 3,500 ng/mL, about 4,000 ng/mL, about 4,500 ng/mL, about 5,000 ng/mL, about 5,500 ng/mL, about 6,000 ng/mL, about 6,500 ng/mL, about 7,000 ng/mL, about 7,500 ng/mL, about 8,000 ng/mL, about 8,500 ng/mL, about 9,000 ng/mL, about 9,500 ng/mL, or about 10,000 ng/mL. In some embodiments, the plasma concentration can be about 10,000 ng/mL, about 15,000 ng/mL, about 20,000 ng/mL, about 25,000 ng/mL, about 30,000 ng/mL, about 35,000 ng/mL, about 40,000 ng/mL, about 45,000 ng/mL, about 50,000 ng/mL, about 55,000 ng/mL, about 60,000 ng/mL, about 65,000 ng/mL, about 70,000 ng/mL, or about 75,000 ng/mL.

The pharmacodynamic parameters can be any parameters suitable for describing compositions of the disclosure. For example, the pharmacodynamic profile can exhibit decreases in viability phenotype for the tumor cells or tumor size reduction in tumor cell lines or xenograft studies, for example, about 24 hours, about 48 hours, about 72 hours, or 1 week.

### EXAMPLES

### Example 1: Compounds of the Disclosure

Indole compounds with alkynyl, aryl, and heteroaryl linkers were prepared. Alkynyl-linked indole compounds are shown in TABLE 1. Aryl-linked indole compounds are shown in TABLE 2. Heteroaryl-linked indole compounds are shown in TABLE 3. The disclosure provides these compounds and a pharmaceutically-acceptable salt thereof.

TABLE 1
Alkynyl indole compounds of the disclosure.

| Mol # | IUPAC name |
|---|---|
| 1. | 1-Anilino-3-{1-ethyl-5-[(1-methyl-4-piperidylamino)methyl]-1H-indol-2-yl}-2-propyne |
| 2. | 1-Anilino-3-{1-ethyl-5-[(methylamino)methyl]-1H-indol-2-yl}-2-propyne |
| 3. | 1-Anilino-3-{1-ethyl-5-[(tetrahydro-2H-pyran-4-ylamino)methyl]-1H-indol-2-yl}-2- |
| | propyne |
| 4. | 1-Anilino-3-[5-(benzylaminomethyl)-1-ethyl-1H-indol-2-yl]-2-propyne |
| 5. | 3-{1-Ethyl-5-[(methylamino)methyl]-1H-indol-2-yl}-1-(p-fluorophenylamino)-2-propyne |
| 6. | 3-{1-Ethyl-5-[(tetrahydro-2H-pyran-4-ylmino)methyl]-1H-indol-2-yl}-1-(p- |
| | fluorophenylamino)-2-propyne |
| 7. | 1-(p-Chlorophenylamino)-3-{1-ethyl-5-[(1-methyl-4-piperidylamino)methyl]-1H-indol-2- |
| | yl}-2-propyne |
| 8. | 3-{1-Ethyl-5-[(tetrahydro-2H-pyran-4-ylamino)methyl]-1H-indol-2-yl}-1-(6-methyl-3- |
| | pyridylamino)-2-propyne |
| 9. | 3-{1-Ethyl-5-[(methylamino)methyl]-1H-indol-2-yl}-1-(6-methyl-3-pyridylamino)-2- |
| | propyne |
| 10. | 3-{1-Ethyl-5-[(1-methyl-4-piperidylamino)methyl]-1H-indol-2-yl}-1-(2-methyl-4- |
| | pyridylamino)-2-propyne |
| 11. | 3-[5-(Benzylaminomethyl)-1-ethyl-1H-indol-2-yl]-1-(2-methyl-4-pyridylamino)-2- |
| | propyne |
| 12. | N-(3-{5-[(Diethylamino)methyl]-1-ethyl-1H-indol-2-yl}prop-2-yn-1-yl)aniline |
| 13. | 4-Chloro-N-(3-{5-[(diethylamino)methyl]-1-ethyl-1H-indol-2-yl}prop-2-yn-1-yl)aniline |
| 14. | N-({1-Ethyl-2-[3-(phenylamino)prop-1-yn-1-yl]-1H-indol-5-yl}methyl)oxetan-3-amine |
| 15. | N-[3-(1-Ethyl-5-{[(2-methylpropyl)amino]methyl}-1H-indol-2-yl)prop-2-yn-1-yl]aniline |
| 16. | N-[3-(1-Ethyl-5-{[(2-methoxyethyl)amino]methyl}-1H-indol-2-yl)prop-2-yn-1-yl]aniline |
| 17. | N-({1-ethyl-2-[3-(phenylamino)prop-1-yn-1-yl]-1H-indol-5-yl}methyl)-1- |
| | methanesulfonylpiperidin-4-amine |
| 18. | N-(3-{1-Ethyl-5-[(ethylamino)methyl]-1H-indol-2-yl}prop-2-yn-1-yl)aniline |
| 19. | N-{3-[5-({[2-(Dimethylamino)ethyl]amino}methyl)-1-ethyl-1H-indol-2-yl]prop-2-yn-1- |
| | yl}aniline |
| 20. | 6-tert-Butyl-N-[3-(1-ethyl-5-{[(1-methylpiperidin-4-yl)amino]methyl}-1H-indol-2- |
| | yl)prop-2-yn-1-yl]pyridin-3-amine |
| 21. | N-[(2-{3-[(4-Chlorophenyl)amino]prop-1-yn-1-yl}-1-ethyl-1H-indol-5-yl)methyl]oxan-4- |
| | amine |
| 22. | 6-tert-Butyl-N-(3-{1-ethyl-5-[(methylamino)methyl]-1H-indol-2-yl}prop-2-yn-1- |
| | yl)pyridin-3-amine |
| 23. | 4-[(3-{1-Ethyl-5-[(methylamino)methyl]-1H-indol-2-yl}prop-2-yn-1- |
| | yl)amino]benzonitrile |
| 24. | 4-tert-Butyl-N-(3-{1-ethyl-5-[(methylamino)methyl]-1H-indol-2-yl}prop-2-yn-1- |
| | yl)benzamide |
| 25. | 4-Chloro-N-(3-{1-ethyl-5-[(methylamino)methyl]-1H-indol-2-yl}prop-2-yn-1-yl)-3- |
| | fluorobenzamide |
| 26. | 4-Cyano-N-({1-ethyl-2-[3-(phenylformamido)prop-1-yn-1-yl]-1H-indol-5-yl}methyl)-N- |
| | methylbenzamide |
| 27. | 3-(3-{1-Ethyl-5-[(methylamino)methyl]-1H-indol-2-yl}prop-2-yn-1-yl)-1-[4- |
| | (trifluoromethyl)phenyl]urea |
| 28. | N-{[1-(2-Chloroethyl)-2-{3-[(4-chlorophenyl)amino]prop-1-yn-1-yl}-1H-indol-5- |
| | yl]methyl}oxan-4-amine |
| 29. | 2-(4-{[3-(1-Ethyl-5-{[(1-methylpiperidin-4-yl)amino]methyl}-1H-indol-2-yl)prop-2-yn- |
| | 1-yl]amino}phenyl)-2-methylpropanenitrile |
| 30. | 4-Cyano-N-(3-{1-ethyl-5-[(methylamino)methyl]-1H-indol-2-yl}prop-2-yn-1- |
| | yl)benzamide |
| 31. | N-(3-{1-Ethyl-5-[(methylamino)methyl]-1H-indol-2-yl}prop-2-yn-1-yl)-6- |
| | methylpyridine-3-carboxamide |
| 32. | 3-[3-(1-Ethyl-5-{[(1-methylpiperidin-4-yl)amino]methyl}-1H-indol-2-yl)prop-2-yn-1-yl]- |
| | 1-phenylurea |
| 33. | N-[(2-{3-[(4-Chloro-3-fluorophenyl)amino]prop-1-yn-1-yl}-1-ethyl-1H-indol-5- |
| | yl)methyl]-1-methylpiperidin-4-amine |
| 34. | 2-(5-{[3-(1-Ethyl-5-{[(1-methylpiperidin-4-yl)amino]methyl}-1H-indol-2-yl)prop-2-yn- |
| | 1-yl]amino}pyridin-2-yl)-2-methylpropanenitrile |
| 35. | N-{[1-(2-Chloroethyl)-2-{3-[(4-chlorophenyl)amino]prop-1-yn-1-yl}-1H-indol-5- |
| | yl]methyl}-1-methylpiperidin-4-amine |
| 36. | 6-tert-Butyl-N-[3-(1-ethyl-5-{[(1-methanesulfonylpiperidin-4-yl)amino]methyl}-1H- |
| | indol-2-yl)prop-2-yn-1-yl]pyridin-3-amine |
| 37. | 2-(4-{[3-(1-Ethyl-5-{[(1-methylpiperidin-4-yl)amino]methyl}-1H-indol-2-yl)prop-2-yn- |
| | 1-yl]amino}phenyl)-2-methylpropanoic acid |
| 38. | 3-(1-Ethyl-5-{[(1-methylpiperidin-4-yl)amino]methyl}-1H-indol-2-yl)-N-methylprop-2- |
| | ynamide |
| 39. | Ethyl 2-(4-{[3-(1-ethyl-5-{[(1-methylpiperidin-4-yl)amino]methyl}-1H-indol-2-yl)prop- |
| | 2-yn-1-yl]amino}phenyl)-2-methylpropanoate |
| 40. | 2-(5-{[3-(1-Ethyl-5-{[(oxan-4-yl)amino]methyl}-1H-indol-2-yl)prop-2-yn-1- |
| | yl]amino}pyridin-2-yl)-2-methylpropanenitrile |
| 41. | N-[(1-Ethyl-2-{3-[(4-methylphenyl)amino]prop-1-yn-1-yl}-1H-indol-5-yl)methyl]-1- |
| | methylpiperidin-4-amine |
| 42. | 4-{[3-(1-Ethyl-5-{[(1-methylpiperidin-4-yl)amino]methyl}-1H-indol-2-yl)prop-2-yn-1- |
| | yl]amino}benzonitrile |
| 43. | 3-(1-Ethyl-5-{[(1-methylpiperidin-4-yl)amino]methyl}-1H-indol-2-yl)-N-phenylprop-2- |
| | ynamide |
| 44. | N-[(2-{3-[(4-Chlorophenyl)amino]prop-1-yn-1-yl}-1-ethyl-1H-indol-5-yl)methyl]-1- |
| | methanesulfonylpiperidin-4-amine |
| 45. | 1-(4-{[(2-{3-[(4-Chlorophenyl)amino]prop-1-yn-1-yl}-1-ethyl-1H-indol-5- |
| | yl)methyl]amino}piperidin-1-yl)ethan-1-one |
| 46. | 6-tert-Butyl-N-[3-(1-ethyl-5-{[(1-methylpiperidin-4-yl)amino]methyl}-1H-indol-2- |
| | yl)prop-2-yn-1-yl]pyridine-3-carboxamide |
| 47. | N-(3-{1-Ethyl-5-[(methylamino)methyl]-1H-indol-2-yl}prop-2-yn-1-yl)-4- |
| | (trifluoromethyl)aniline |
| 48. | N-[(1-Ethyl-2-{3-[(4-methylphenyl)amino]prop-1-yn-1-yl}-1H-indol-5-yl)methyl]oxan- |
| | 4-amine |
| 49. | N-(3-{1-ethyl-4-[(methylamino)methyl]-1H-indol-2-yl}prop-2-yn-1-yl)aniline |
| 50. | N-[3-(1-ethyl-4-{[4-(pyrrolidin-1-yl)piperidin-1-yl]methyl}-1H-indol-2-yl)prop-2-yn-1- |
| | yl]aniline |
| 51. | N-({1-ethyl-2-[3-(phenylamino)prop-1-yn-1-yl]-1H-indol-4-yl}methyl)-1- |
| | methylpiperidin-4-amine |
| 52. | 1-[(2-{3-[(4-chlorophenyl)amino]prop-1-yn-1-yl}-1-ethyl-1H-indol-4- |
| | yl)methyl]piperidin-4-ol |
| 53. | 4-Chloro-N-[3-(1-ethyl-4-{[4-(pyrrolidin-1-yl)piperidin-1-yl]methyl}-1H-indol-2- |
| | yl)prop-2-yn-1-yl]aniline |
| 54. | 1-[(2-{3-[(4-chlorophenyl)amino]prop-1-yn-1-yl}-1-ethyl-1H-indol-4-yl)methyl]-N,N- |
| | dimethylpiperidin-4-amine |
| 55. | 4-Chloro-N-(3-{1-ethyl-4-[(4-methylpiperazin-1-yl)methyl]-1H-indol-2-yl}prop-2-yn-1- |
| | yl)aniline |
| 56. | 1-{1-[(2-{3-[(4-Chlorophenyl)amino]prop-1-yn-1-yl}-1-ethyl-1H-indol-4- |
| | yl)methyl]piperidin-4-yl}piperidin-4-ol |
| 57. | 2-(5-{[3-(4-{[4-(4-Aminopiperidin-1-yl)piperidin-1-yl]methyl}-1-ethyl-1H-indol-2- |
| | yl)prop-2-yn-1-yl]amino]pyridin-2-yl)-2-methylpropanenitrile |
| 58. | 1-[(1-ethyl-2-{3-[(4-fluorophenyl)amino]prop-1-yn-1-yl}-1H-indol-5-yl)methyl]-N,N- |
| | dimethylpiperidin-4-amine |
| 59. | 4-N-({1-ethyl-2-[3-(phenylamino)prop-1-yn-1-yl]-1H-indol-5-yl}methyl)-1-N,1-N- |
| | dimethylcyclohexane-1,4-diamine |
| 60. | 4-chloro-N-(3-{1-ethyl-5-[(methylamino)methyl]-1H-indol-2-yl}prop-2-yn-1-yl)-3- |
| | fluoroaniline |
| 61. | 6-tert-butyl-N-(3-{1-ethyl-5-[(methylamino)methyl]-1H-indol-2-yl}prop-2-yn-1- |
| | yl)pyridine-3-carboxamide |
| 62. | N-(3-{1-ethyl-5-[(methylamino)methyl]-1H-indol-2-yl}prop-2-yn-1-yl)benzamide |
| 63. | 3-(3-{1-ethyl-5-[(methylamino)methyl]-1H-indol-2-yl}prop-2-yn-1-yl)-1-(4- |
| | methylphenyl)urea |
| 64. | 4-chloro-N-(3-{1-ethyl-5-[(methylamino)methyl]-1H-indol-2-yl}prop-2-yn-1-yl)aniline |
| 65. | 4-{[3-(1-ethyl-5-{[(oxan-4-yl)amino]methyl}-1H-indol-2-yl)prop-2-yn-1- |
| | yl]amino}benzonitrile |
| 66. | N-[(2-{3-[(4-chloro-3-fluorophenyl)amino]prop-1-yn-1-yl}-1-ethyl-1H-indol-5- |
| | yl)methyl]oxan-4-amine |
| 67. | 3-[3-(1-ethyl-5-{[(oxan-4-yl)amino]methyl}-1H-indol-2-yl)prop-2-yn-1-yl]-1-phenylurea |
| 68. | 6-tert-butyl-N-[3-(1-ethyl-5-{[(oxan-4-yl)amino]methyl}-1H-indol-2-yl)prop-2-yn-1- |
| | yl]pyridin-3-amine |
| 69. | 4-{[(2-{3-[(4-chlorophenyl)amino]prop-1-yn-1-yl}-1-ethyl-1H-indol-5- |
| | yl)methyl]amino}-1λ⁶-thiane-1,1-dione |
| 70. | N-[(2-{3-[(4-chlorophenyl)amino]prop-1-yn-1-yl}-1-ethyl-1H-indol-5-yl)methyl]-1-(2- |
| | methanesulfonylethyl)piperidin-4-amine |
| 71. | 1-(4-{[(2-{3-[(4-chlorophenyl)amino]prop-1-yn-1-yl}-1-ethyl-1H-indol-5- |
| | yl)methyl]amino}piperidin-1-yl)-2-(dimethylamino)ethan-1-one |
| 72. | 2-(4-{[(2-{3-[(4-chlorophenyl)amino]prop-1-yn-1-yl}-1-ethyl-1H-indol-5- |
| | yl)methyl]amino}piperidin-1-yl)-N,N-dimethylacetamide |
| 73. | 2-tert-butyl-N-[3-(1-ethyl-5-{[(1-methylpiperidin-4-yl)amino]methyl}-1H-indol-2- |
| | yl)prop-2-yn-1-yl]pyrimidin-5-amine |
| 74. | 2-methyl-2-(5-{[3-(5-{[(oxan-4-yl)amino]methyl}-1-(2,2,2-trifluoroethyl)-1H-indol-2- |
| | yl)prop-2-yn-1-yl]amino}pyridin-2-yl)propanenitrile |
| 75. | 2-[5-({3-[1-(2-fluoroethyl)-5-{[(oxan-4-yl)amino]methyl}-1H-indol-2-yl]prop-2-yn-1- |
| | yl}amino)pyridin-2-yl]-2-methylpropanenitrile |
| 76. | 3-(1-ethyl-5-{[(1-methylpiperidin-4-yl)amino]methyl}-1H-indol-2-yl)prop-2-yn-1-ol |
| 77. | 2-[5-({3-[1-(2-chloroethyl)-5-{[(oxan-4-yl)amino]methyl}-1H-indol-2-yl]prop-2-yn-1- |
| | yl}amino)pyridin-2-yl]-2-methylpropanenitrile |
| 78. | 2-[5-({3-[1-(2,2-difluoroethyl)-5-{[(oxan-4-yl)amino]methyl}-1H-indol-2-yl]prop-2-yn- |
| | 1-yl}amino)pyridin-2-yl]-2-methylpropanenitrile |
| 79. | 6-chloro-N-[3-(1-ethyl-5-{[(1-methylpiperidin-4-yl)amino]methyl}-1H-indol-2-yl)prop- |
| | 2-yn-1-yl]pyridin-3-amine |
| 80. | tert-butyl N-({3-ethyl-2-[3-(phenylamino)prop-1-yn-1-yl]-1H-indol-6-yl}methyl)-N- |
| | (oxan-4-yl)carbamate |
| 81. | 6-Chloro-N-[3-(1-ethyl-4-{[4-(pyrrolidin-1-yl)piperidin-1-yl]methyl}-1H-indol-2- |
| | yl)prop-2-yn-1-yl]pyridin-3-amine |
| 82. | 3-(1-ethyl-5-{[(1-methylpiperidin-4-yl)amino]methyl}-1H-indol-2-yl)prop-2-yn-1-yl |
| | benzoate |
| 83. | 2-[5-({3-[1-(2-chloroethyl)-5-{[(1-methylpiperidin-4-yl)amino]methyl}-1H-indol-2- |
| | yl]prop-2-yn-1-yl}amino)pyridin-2-yl]-2-methylpropanenitrile |
| 84. | N-(6-chloropyridin-3-yl)-3-(1-ethyl-5-{[(1-methylpiperidin-4-yl)amino]methyl}-1H- |
| | indol-2-yl)prop-2-ynamide |
| 85. | N-[6-(1-cyano-1-methylethyl)pyridin-3-yl]-3-(1-ethyl-5-{[(1-methylpiperidin-4- |
| | yl)amino]methyl}-1H-indol-2-yl)prop-2-ynamide |
| 86. | N-({3-ethyl-2-[3-(phenylamino)prop-1-yn-1-yl]-1H-indol-6-yl}methyl)oxan-4-amine |
| 87. | 2-[5-({3-[1-(2-chloroethyl)-4-{[4-(pyrrolidin-1-yl)piperidin-1-yl]methyl}-1H-indol-2- |
| | yl]prop-2-yn-1-yl}amino)pyridin-2-yl]-2-methylpropanenitrile |
| 88. | 2-(5-{[3-(5-{[(1-methanesulfonylpiperidin-4-yl)amino]methyl}-1-(2,2,2-trifluoroethyl)- |
| | 1H-indol-2-yl)prop-2-yn-1-yl]amino}pyridin-2-yl)-2-methylpropanenitrile |
| 89. | 2-[5-({3-[5-({[1-(2-methanesulfonylethyl)piperidin-4-yl]amino}methyl)-1-(2,2,2- |
| | trifluoroethyl)-1H-indol-2-yl]prop-2-yn-1-yl}amino)pyridin-2-yl]-2-methylpropanenitrile |
| 90. | 2-(5-{[3-(5-{[(1-acetylpiperidin-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 91. | 2-{5-[(3-{5-[({1-[2-(dimethylamino)acetyl]piperidin-4-yl}amino)methyl]-1-(2,2,2- |
| | trifluoroethyl)-1H-indol-2-yl}prop-2-yn-1-yl)amino]pyridin-2-yl}-2-methylpropanenitrile |
| 92. | 2-methyl-2-(5-{[3-(5-{[(1-methylpiperidin-4-yl)amino]methyl}-1-(2,2,2-trifluoroethyl)- |
| | 1H-indol-2-yl)prop-2-yn-1-yl]amino}pyridin-2-yl)propanenitrile |
| 93. | 2-methyl-2-{5-[(3-{5-[(methylamino)methyl]-1-(2,2,2-trifluoroethyl)-1H-indol-2- |
| | yl}prop-2-yn-1-yl)amino]pyridin-2-yl}propanenitrile |
| 94. | 6-Chloro-N-[3-(5-{[(1-methylpiperidin-4-yl)amino]methyl}-1-(2,2,2-trifluoroethyl)-1H- |
| | indol-2-yl)prop-2-yn-1-yl]pyridin-3-amine |
| 95. | 6-chloro-N-[3-(5-{[(oxan-4-yl)amino]methyl}-1-(2,2,2-trifluoroethyl)-1H-indol-2- |
| | yl)prop-2-yn-1-yl]pyridin-3-amine |
| 96. | 2-[5-({3-[1-(cyclopropylmethyl)-5-{[(oxan-4-yl)amino]methyl}-1H-indol-2-yl]prop-2-yn- |
| | 1-yl}amino)pyridin-2-yl]-2-methylpropanenitrile |
| 97. | 2-(5-{[3-(4-{[4-(diethylamino)piperidin-1-yl]methyl}-1-(2,2,2-trifluoroethyl)-1H-indol-2- |
| | yl)prop-2-yn-1-yl]amino}pyridin-2-yl)-2-methylpropanenitrile |
| 98. | 2-methyl-2-{5-[(3-{4-[(4-methylpiperazin-1-yl)methyl]-1-(2,2,2-trifluoroethyl)-1H-indol- |
| | 2-yl}prop-2-yn-1-yl)amino]pyridin-2-yl}propanenitrile |
| 99. | 2-(5-{[3-(1-ethyl-7-fluoro-4-{[4-(pyrrolidin-1-yl)piperidin-1-yl]methyl}-1H-indol-2- |
| | yl)prop-2-yn-1-yl]amino}pyridin-2-yl)-2-methylpropanenitrile |
| 100. | 2-methyl-2-(5-{[3-(4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)propanenitrile |
| 101. | 2-(5-{[3-(4-{[4-(4-hydroxypiperidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 102. | N-(6-cyanopyridin-3-yl)-3-(1-ethyl-5-{[(1-methylpiperidin-4-yl)amino]methyl}-1H- |
| | indol-2-yl)prop-2-ynamide |
| 103. | N-[6-(1-cyano-1-methylethyl)pyridin-3-yl]-3-(5-{[(oxan-4-yl)amino]methyl}-1-(2,2,2- |
| | trifluoroethyl)-1H-indol-2-yl)prop-2-ynamide |
| 104. | N-[6-(1-cyano-1-methylethyl)pyridin-3-yl]-3-(5-{[(1-methylpiperidin-4- |
| | yl)amino]methyl}-1-(2,2,2-trifluoroethyl)-1H-indol-2-yl)prop-2-ynamide |
| 105. | 2-methyl-2-(5-{[3-(5-{[(oxan-4-yl)amino]methyl}-1-(oxiran-2-ylmethyl)-1H-indol-2- |
| | yl)prop-2-yn-1-yl]amino}pyridin-2-yl)propanenitrile |
| 106. | 2-(5-{[3-(5-{[(2-methoxyethyl)amino]methyl}-1-(2,2,2-trifluoroethyl)-1H-indol-2- |
| | yl)prop-2-yn-1-yl]amino}pyridin-2-yl)-2-methylpropanenitrile |
| 107. | 2-methyl-2-[5-({3-[5-({[2-(morpholin-4-yl)ethyl]amino}methyl)-1-(2,2,2-trifluoroethyl)- |
| | 1H-indol-2-yl]prop-2-yn-1-yl}amino)pyridin-2-yl]propanenitrile |
| 108. | 2-methyl-2-(5-{[3-(4-{[(1-methylpiperidin-4-yl)amino]methyl}-1-(2,2,2-trifluoroethyl)- |
| | 1H-indol-2-yl)prop-2-yn-1-yl]amino}pyridin-2-yl)propanenitrile |
| 109. | 2-methyl-2-(5-{[3-(4-{[(oxan-4-yl)amino]methyl}-1-(2,2,2-trifluoroethyl)-1H-indol-2- |
| | yl)prop-2-yn-1-yl]amino}pyridin-2-yl)propanenitrile |
| 110. | 2-[5-({3-[5-({[2-(dimethylamino)ethyl]amino}methyl)-1-(2,2,2-trifluoroethyl)-1H-indol- |
| | 2-yl]prop-2-yn-1-yl}amino)pyridin-2-yl]-2-methylpropanenitrile |
| 111. | 2-(5-{[3-(7-fluoro-4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 112. | 2-methyl-2-[5-({3-[1-(2,2,2-trifluoroethyl)-5-{[(2,2,2-trifluoroethyl)amino]methyl}-1H- |
| | indol-2-yl]prop-2-yn-1-yl}amino)pyridin-2-yl]propanenitrile |
| 113. | 2-[5-({3-[5-({[1-(2-hydroxyethyl)piperidin-4-yl]amino}methyl)-1-(2,2,2-trifluoroethyl)- |
| | 1H-indol-2-yl]prop-2-yn-1-yl}amino)pyridin-2-yl]-2-methylpropanenitrile |
| 114. | 2-[5-({3-[5-({[1-(2-methoxyethyl)piperidin-4-yl]amino}methyl)-1-(2,2,2-trifluoroethyl)- |
| | 1H-indol-2-yl]prop-2-yn-1-yl}amino)pyridin-2-yl]-2-methylpropanenitrile |
| 115. | 2-[5-({3-[5-({[4-(dimethylamino)cyclohexyl]amino}methyl)-1-(2,2,2-trifluoroethyl)-1H- |
| | indol-2-yl]prop-2-yn-1-yl}amino)pyridin-2-yl]-2-methylpropanenitrile |
| 116. | 2-methyl-2-{5-[(3-{5-[({1-[2-(morpholin-4-yl)acetyl]piperidin-4-yl}amino)methyl]-1- |
| | (2,2,2-trifluoroethyl)-1H-indol-2-yl}prop-2-yn-1-yl)amino]pyridin-2-yl}propanenitrile |
| 117. | 2-(5-{[3-(4-{[(2-methoxyethyl)amino]methyl}-1-(2,2,2-trifluoroethyl)-1H-indol-2- |
| | yl)prop-2-yn-1-yl]amino}pyridin-2-yl)-2-methylpropanenitrile |
| 118. | 2-methyl-2-{5-[(3-{4-[(methylamino)methyl]-1-(2,2,2-trifluoroethyl)-1H-indol-2- |
| | yl}prop-2-yn-1-yl)amino]pyridin-2-yl}propanenitrile |
| 119. | 2-{5-[(3-{4-[(4-acetylpiperazin-1-yl)methyl]-1-(2,2,2-trifluoroethyl)-1H-indol-2-yl}prop- |
| | 2-yn-1-yl)amino]pyridin-2-yl}-2-methylpropanenitrile |
| 120. | 2-methyl-2-[5-({3-[4-(morpholin-4-ylmethyl)-1-(2,2,2-trifluoroethyl)-1H-indol-2-yl]prop- |
| | 2-yn-1-yl}amino)pyridin-2-yl]propanenitrile |
| 121. | 2-(5-{[3-(4-{[4-(dimethylamino)piperidin-1-yl]methyl}-1-(2,2,2-trifluoroethyl)-1H-indol- |
| | 2-yl)prop-2-yn-1-yl]amino}pyridin-2-yl)-2-methylpropanenitrile |
| 122. | 2-[5-({3-[4-(hydroxymethyl)-1-(2,2,2-trifluoroethyl)-1H-indol-2-yl]prop-2-yn-1- |
| | yl}amino)pyridin-2-yl]-2-methylpropanenitrile |
| 123. | 2-methyl-2-[5-({3-[4-({4-[2-(morpholin-4-yl)-2-oxoethyl]piperazin-1-yl}methyl)-1- |
| | (2,2,2-trifluoroethyl)-1H-indol-2-yl]prop-2-yn-1-yl}amino)pyridin-2-yl]propanenitrile |
| 124. | 2-(5-{[3-(3-ethyl-7-{[4-(pyrrolidin-1-yl)piperidin-1-yl]methyl}-1H-indol-2-yl)prop-2-yn- |
| | 1-yl]amino}pyridin-2-yl)-2-methylpropanenitrile |
| 125. | methyl 5-{[3-(5-{[(oxan-4-yl)amino]methyl}-1-(2,2,2-trifluoroethyl)-1H-indol-2-yl)prop- |
| | 2-yn-1-yl]amino}pyridine-2-carboxylate |
| 126. | N-methyl-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 127. | N-(2-hydroxyethyl)-5-{[3-(5-{[(oxan-4-yl)amino]methyl}-1-(2,2,2-trifluoroethyl)-1H- |
| | indol-2-yl)prop-2-yn-1-yl]amino}pyridine-2-carboxamide |
| 128. | N-(2-methoxyethyl)-5-{[3-(5-{[(oxan-4-yl)amino]methyl}-1-(2,2,2-trifluoroethyl)-1H- |
| | indol-2-yl)prop-2-yn-1-yl]amino}pyridine-2-carboxamide |
| 129. | 2-[(5-{[3-(5-{[(oxan-4-yl)amino]methyl}-1-(2,2,2-trifluoroethyl)-1H-indol-2-yl)prop-2- |
| | yn-1-yl]amino}pyridin-2-yl)formamido]acetic acid |
| 130. | 5-{[3-(5-{[(oxan-4-yl)amino]methyl}-1-(2,2,2-trifluoroethyl)-1H-indol-2-yl)prop-2-yn-1- |
| | yl]amino}pyridine-2-carboxylic acid |
| 131. | N-(2-methanesulfonylethyl)-5-{[3-(5-{[(oxan-4-yl)amino]methyl}-1-(2,2,2- |
| | trifluoroethyl)-1H-indol-2-yl)prop-2-yn-1-yl]amino}pyridine-2-carboxamide |
| 132. | 2-[5-({3-[1-(cyanomethyl)-5-{[(oxan-4-yl)amino]methyl}-1H-indol-2-yl]prop-2-yn-1- |
| | yl}amino)pyridin-2-yl]-2-methylpropanenitrile |
| 133. | 2-methyl-2-[5-({3-[1-(2-methylpropyl)-5-{[(oxan-4-yl)amino]methyl}-1H-indol-2- |
| | yl]prop-2-yn-1-yl}amino)pyridin-2-yl]propanenitrile |
| 134. | 2-methyl-2-{5-[(3-{4-[(oxan-4-yl)amino]-1-(2,2,2-trifluoroethyl)-1H-indol-2-yl}prop-2- |
| | yn-1-yl)amino]pyridin-2-yl}propanenitrile |
| 135. | 5-{[3-(5-{[(oxan-4-yl)amino]methyl}-1-(2,2,2-trifluoroethyl)-1H-indol-2-yl)prop-2-yn-1- |
| | yl]amino}pyridine-2-carbonitrile |
| 136. | N,N-dimethyl-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 137. | N-(oxan-4-yl)-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 138. | 2-tert-butyl-N-[3-(5-{[(oxan-4-yl)amino]methyl}-1-(2,2,2-trifluoroethyl)-1H-indol-2- |
| | yl)prop-2-yn-1-yl]pyrimidin-5-amine |
| 139. | N-(1-methylpiperidin-4-yl)-5-{[3-(5-{[(oxan-4-yl)amino]methyl}-1-(2,2,2-trifluoroethyl)- |
| | 1H-indol-2-yl)prop-2-yn-1-yl]amino}pyridine-2-carboxamide |
| 140. | N-[6-(1-cyano-1-methylethyl)pyridin-3-yl]-3-[1-(2-fluoroethyl)-5-{[(oxan-4- |
| | yl)amino]methyl}-1H-indol-2-yl]prop-2-ynamide |
| 141. | 2-(5-{[3-(7-chloro-4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 142. | 2-(5-{[3-(6-fluoro-4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 143. | 2-(5-{[3-(1-ethyl-5-{[(oxan-4-yl)amino]methyl}-1H-pyrrolo[2,3-c]pyridin-2-yl)prop-2- |
| | yn-1-yl]amino}pyridin-2-yl)-2-methylpropanenitrile |
| 144. | 2-methyl-2-(5-{[3-(5-{[(oxan-4-yl)amino]methyl}-1-(2,2,2-trifluoroethyl)-1H- |
| | pyrrolo[2,3-c]pyridin-2-yl)prop-2-yn-1-yl]amino}pyridin-2-yl)propanenitrile |
| 145. | 2-(5-{[3-(5-{[4-(dimethylamino)piperidin-1-yl]methyl}-1-(2,2,2-trifluoroethyl)-1H- |
| | pyrrolo[2,3-c]pyridin-2-yl)prop-2-yn-1-yl]amino}pyridin-2-yl)-2-methylpropanenitrile |
| 146. | 2-(5-{[3-(7-chloro-4-{[4-(pyrrolidin-1-yl)piperidin-1-yl]methyl}-1-(2,2,2-trifluoroethyl)- |
| | 1H-pyrrolo[2,3-c]pyridin-2-yl)prop-2-yn-1-yl]amino}pyridin-2-yl)-2- |
| | methylpropanenitrile |
| 147. | 2-(5-{[3-(4-{[4-(dimethylamino)-piperidin-1-yl]methyl}-6-fluoro-1-(2,2,2-trifluoroethyl)- |
| | 1H-indol-2-yl)prop-2-yn-1-yl]amino}pyridin-2-yl)-2-methylpropanenitrile |
| 148. | 2-(5-{[3-(4-{[4-(diethylamino)piperidin-1-yl]methyl}-6-fluoro-1-(2,2,2-trifluoroethyl)- |
| | 1H-indol-2-yl)prop-2-yn-1-yl]amino}pyridin-2-yl)-2-methylpropanenitrile |
| 149. | 2-(5-{[3-(6-fluoro-4-{[4-(4-hydroxypiperidin-1-yl)piperidin-1-yl]methyl}-1-(2,2,2- |
| | trifluoroethyl)-1H-indol-2-yl)prop-2-yn-1-yl]amino}pyridin-2-yl)-2-methylpropanenitrile |
| 150. | 2-(5-{[3-(6-fluoro-5-{[(oxan-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 151. | 2-(5-{[3-(5-{[(1-acetylpiperidin-4-yl)amino]methyl}-6-fluoro-1-(2,2,2-trifluoroethyl)-1H- |
| | indol-2-yl)prop-2-yn-1-yl]amino}pyridin-2-yl)-2-methylpropanenitrile |
| 152. | 2-(5-{[3-(6-chloro-4-{[4-(dimethylamino)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 153. | 2-(5-{[3-(6-chloro-4-{[4-(diethylamino)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 154. | 2-(5-{[3-(6-chloro-4-{[4-(4-hydroxypiperidin-1-yl)piperidin-1-yl]methyl}-1-(2,2,2- |
| | trifluoroethyl)-1H-indol-2-yl)prop-2-yn-1-yl]amino}pyridin-2-yl)-2-methylpropanenitrile |
| 155. | 2-(5-{[3-(4-{[4-(2-methanesulfonyl-ethyl)piperazin-1-yl]methyl}-1-(2,2,2-trifluoroethyl)- |
| | 1H-indol-2-yl)prop-2-yn-1-yl]amino}pyridin-2-yl)-2-methylpropanenitrile |
| 156. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperazin-1-yl)-N,N-dimethylacetamide |
| 157. | 2-methyl-2-{5-[(3-{4-[(3-oxopiperazin-1-yl]methyl]-1-(2,2,2-trifluoroethyl)-1H-indol-2- |
| | yl}prop-2-yn-1-yl)amino]pyridin-2-yl}propanenitrile |
| 158. | 2-methyl-2-[5-({3-[4-({4-[2-(morpholin-4-yl)-2-oxoethyl]piperidin-1-yl}methyl)-1- |
| | (2,2,2-trifluoroethyl)-1H-indol-2-yl]prop-2-yn-1-yl}amino)pyridin-2-yl]propanenitrile |
| 159. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperazin-1-yl)acetamide |
| 160. | N-[6-(1-cyano-1-methylethyl)pyridin-3-yl]-3-(4-{[4-(pyrrolidin-1-yl)piperidin-1- |
| | yl]methyl}-1-(2,2,2-trifluoroethyl)-1H-indol-2-yl)prop-2-ynamide |
| 161. | 2-(1-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperidin-4-yl)acetamide |
| 162. | 2-(5-{[3-(4-{[4-(2-aminoethyl)-piperazin-1-yl]methyl}-1-(2,2,2-trifluoroethyl)-1H-indol- |
| | 2-yl)prop-2-yn-1-yl]amino}pyridin-2-yl)-2-methylpropanenitrile |
| 163. | 2-(1-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperidin-4-yl)-N,N-dimethylacetamide |
| 164. | 2-methyl-2-(5-{[3-(4-{[4-(morpholin-4-yl)piperidin-1-yl]methyl}-1-(2,2,2-trifluoroethyl)- |
| | 1H-indol-2-yl)prop-2-yn-1-yl]amino}pyridin-2-yl)propanenitrile |
| 165. | 2-(5-{[3-(4-{[4-(4-aminopiperidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 166. | 2-methyl-2-[5-({3-[1-(oxiran-2-ylmethyl)-4-{[4-(pyrrolidin-1-yl)piperidin-1-yl]methyl}- |
| | 1H-indol-2-yl]prop-2-yn-1-yl}amino)pyridin-2-yl]propanenitrile |
| 167. | 2-(5-{[3-(3-ethyl-6-{[(oxan-4-yl)amino]methyl}-1H-indol-2-yl)prop-2-yn-1- |
| | yl]amino}pyridin-2-yl)-2-methylpropanenitrile |
| 168. | 2-methyl-2-(5-{[3-(6-{[(oxan-4-yl)amino]methyl}-3-(2,2,2-trifluoroethyl)-1H-indol-2- |
| | yl)prop-2-yn-1-yl]amino}pyridin-2-yl)propanenitrile |
| 169. | 2-(5-{[3-(1-acetyl-3-ethyl-6-{[(oxan-4-yl)amino]methyl}-1H-indol-2-yl)prop-2-yn-1- |
| | yl]amino}pyridin-2-yl)-2-methylpropanenitrile |
| 170. | 2-(5-{[3-(3-ethyl-6-{[(1-methylpiperidin-4-yl)amino]methyl}-1H-indol-2-yl)prop-2-yn-1- |
| | yl]amino}pyridin-2-yl)-2-methylpropanenitrile |
| 171. | 2-methyl-2-(5-{[3-(6-{[(1-methylpiperidin-4-yl)amino]methyl}-3-(2,2,2-trifluoroethyl)- |
| | 1H-indol-2-yl)prop-2-yn-1-yl]amino}pyridin-2-yl)propanenitrile |
| 172. | 2-{5-[(3-{6-chloro-4-[(1-methylpiperidin-4-yl)amino]-1-(2,2,2-trifluoroethyl)-1H-indol- |
| | 2-yl}prop-2-yn-1-yl)amino]pyridin-2-yl}-2-methylpropanenitrile |
| 173. | 2-(3-{[6-(1-cyano-1-methylethyl)pyridin-3-yl]amino}prop-1-yn-1-yl)-6-fluoro-N-(1- |
| | methylpiperidin-4-yl)-1-(2,2,2-trifluoroethyl)-1H-indole-4-carboxamide |
| 174. | 2-[5-({3-[6-fluoro-4-(4-methylpiperazine-1-carbonyl)-1-(2,2,2-trifluoroethyl)-1H-indol-2- |
| | yl]prop-2-yn-1-yl}amino)pyridin-2-yl]-2-methylpropanenitrile |
| 175. | 6-fluoro-2-{3-[(6-methanesulfonylpyridin-3-yl)amino]prop-1-yn-1-yl}-N-(1- |
| | methylpiperidin-4-yl)-1-(2,2,2-trifluoroethyl)-1H-indol-4-amine |
| 176. | 2-{5-[(3-{6-fluoro-4-[(1-methylpiperidin-4-yl)amino]-1-(2,2,2-trifluoroethyl)-1H-indol-2- |
| | yl}prop-2-yn-1-yl)amino]pyridin-2-yl}-2-methylpropanenitrile |
| 177. | 5-[(3-{6-fluoro-4-[(1-methylpiperidin-4-yl)amino]-1-(2,2,2-trifluoroethyl)-1H-indol-2- |
| | yl}prop-2-yn-1-yl)amino]-N-(pyridin-3-yl)pyridine-2-carboxamide |
| 178. | 2-methyl-2-(5-{[3-(5-{[(oxan-4-yl)amino]methyl}-1-(2,2,2-trifluoroethyl)-1H- |
| | pyrrolo[2,3-b]pyridin-2-yl)prop-2-yn-1-yl]amino}pyridin-2-yl)propanenitrile |
| 179. | 2-methyl-2-{5-[(3-{4-[(1-methylpiperidin-4-yl)amino]-1-(2,2,2-trifluoroethyl)-1H- |
| | pyrrolo[2,3-b]pyridin-2-yl}prop-2-yn-1-yl)amino]pyridin-2-yl}propanenitrile |
| 180. | 2-(5-{[3-(7-chloro-1-ethyl-4-{[4-(pyrrolidin-1-yl)piperidin-1-yl]methyl}-1H-indol-2- |
| | yl)prop-2-yn-1-yl]amino}pyridin-2-yl)-2-methylpropanenitrile |
| 181. | 2-(5-{[3-(7-chloro-5-{[(oxan-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 182. | 2-(5-{[3-(7-chloro-5-{[(1-methylpiperidin-4-yl)amino]methyl}-1-(2,2,2-trifluoroethyl)- |
| | 1H-indol-2-yl)prop-2-yn-1-yl]amino}pyridin-2-yl)-2-methylpropanenitrile |
| 183. | 2-{5-[(3-{7-fluoro-4-[(1-methylpiperidin-4-yl)amino]-1-(2,2,2-trifluoroethyl)-1H-indol-2- |
| | yl}prop-2-yn-1-yl)amino]pyridin-2-yl}-2-methylpropanenitrile |
| 184. | 2-(5-{[3-(7-fluoro-5-{[(oxan-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 185. | 2-(5-{[3-(7-fluoro-5-{[(1-methylpiperidin-4-yl)amino]methyl}-1-(2,2,2-trifluoroethyl)- |
| | 1H-indol-2-yl)prop-2-yn-1-yl]amino}pyridin-2-yl)-2-methylpropanenitrile |
| 186. | 2-methyl-2-(5-{[3-(5-{[(oxan-4-yl)amino]methyl}-1-(2,2,2-trifluoroethyl)-1H-1,3- |
| | benzodiazol-2-yl)prop-2-yn-1-yl]amino}pyridin-2-yl)propanenitrile |
| 187. | N-{[2-(2-phenylethynyl)-1-(2,2,2-trifluoroethyl)-1H-1,3-benzodiazol-5-yl]methyl}oxan- |
| | 4-amine |
| 188. | 2-methyl-2-(5-{[3-(5-{[(oxan-4-yl)amino]methyl}-1-(2,2,2-trifluoroethyl)-1H- |
| | pyrrolo[3,2-b]pyridin-2-yl)prop-2-yn-1-yl]amino}pyridin-2-yl)propanenitrile |
| 189. | 2-{3-[(4-methanesulfonylphenyl)amino]prop-1-yn-1-yl}-5-methyl-N-(1-methylpiperidin- |
| | 4-yl)-1-(2,2,2-trifluoroethyl)-1H-indol-4-amine |
| 190. | 4-[(2-{3-[(4-methanesulfonylphenyl)amino]prop-1-yn-1-yl}-5-methyl-1-(2,2,2- |
| | trifluoroethyl)-1H-indol-4-yl)amino]-1λ⁶-thiane-1,1-dione |
| 191. | 2-methyl-2-{5-[(3-{5-methyl-4-[(1-methylpiperidin-4-yl)amino]-1-(2,2,2-trifluoroethyl)- |
| | 1H-indol-2-yl}prop-2-yn-1-yl)amino]pyridin-2-yl}propanenitrile |
| 192. | N-[1-(2-methanesulfonylethyl)piperidin-4-yl]-2-{3-[(4- |
| | methanesulfonylphenyl)amino]prop-1-yn-1-yl}-5-methyl-1-(2,2,2-trifluoroethyl)-1H- |
| | indol-4-amine |
| 193. | 4-[(3-{5-methyl-4-[(piperidin-4-yl)amino]-1-(2,2,2-trifluoroethyl)-1H-indol-2-yl}prop-2- |
| | yn-1-yl)amino]benzene-1-sulfonamide |
| 194. | 2-{3-[(4-methanesulfonylphenyl)amino]prop-1-yn-1-yl}-5-methyl-N-[1-(oxan-4- |
| | yl)piperidin-4-yl]-1-(2,2,2-trifluoroethyl)-1H-indol-4-amine |
| 195. | 2-{4-[(2-{3-[(4-methanesulfonylphenyl)amino]prop-1-yn-1-yl}-5-methyl-1-(2,2,2- |
| | trifluoroethyl)-1H-indol-4-yl)amino]piperidin-1-yl}ethan-1-ol |
| 196. | 2-[5-({3-[4-(methoxymethyl)-1-(2,2,2-trifluoroethyl)-1H-indol-2-yl]prop-2-yn-1- |
| | yl}amino)pyridin-2-yl]-2-methylpropanenitrile |
| 197. | 2-[5-({3-[4-(cyanomethyl)-1-(2,2,2-trifluoroethyl)-1H-indol-2-yl]prop-2-yn-1- |
| | yl}amino)pyridin-2-yl]-2-methylpropanenitrile |
| 198. | 2-methyl-2-[5-({3-[5-(morpholine-4-carbonyl)-1-(2,2,2-trifluoroethyl)-1H-indol-2- |
| | yl]prop-2-yn-1-yl}amino)pyridin-2-yl]propanenitrile |
| 199. | 2-methyl-2-[5-({3-[5-(4-methylpiperazine-1-carbonyl)-1-(2,2,2-trifluoroethyl)-1H-indol- |
| | 2-yl]prop-2-yn-1-yl}amino)pyridin-2-yl]propanenitrile |
| 200. | 2-{5-[(3-{5-[4-(dimethylamino)piperidine-1-carbonyl]-1-(2,2,2-trifluoroethyl)-1H-indol- |
| | 2-yl}prop-2-yn-1-yl)amino]pyridin-2-yl}-2-methylpropanenitrile |
| 201. | 2-(3-{[6-(1-cyano-1-methylethyl)pyridin-3-yl]amino}prop-1-yn-1-yl)-N-{1-[2- |
| | (dimethylamino)acetyl]piperidin-4-yl}-1-(2,2,2-trifluoroethyl)-1H-indole-5-carboxamide |
| 202. | 2-(3-{[6-(1-cyano-1-methylethyl)pyridin-3-yl]amino}prop-1-yn-1-yl)-N-(oxan-4-yl)-1- |
| | (2,2,2-trifluoroethyl)-1H-indole-5-carboxamide |
| 203. | 2-methyl-2-(5-{[3-(5-{1-[(oxan-4-yl)amino]ethyl}-1-(2,2,2-trifluoroethyl)-1H-indol-2- |
| | yl)prop-2-yn-1-yl]amino}pyridin-2-yl)propanenitrile |
| 204. | 2-methyl-2-{5-[(3-{4-[(1-methylpiperidin-4-yl)amino]-1-(2,2,2-trifluoroethyl)-1H- |
| | pyrrolo[3,2-c]pyridin-2-yl}prop-2-yn-1-yl)amino]pyridin-2-yl}propanenitrile |
| 205. | 2-methyl-2-[5-({3-[5-(morpholin-4-ylmethyl)-1-(2,2,2-trifluoroethyl)-1H-indol-2-yl]prop- |
| | 2-yn-1-yl}amino)pyridin-2-yl]propanenitrile |
| 206. | 2-[5-({3-[5-({[1-(2-cyanoethyl)-piperidin-4-yl]amino}methyl)-1-(2,2,2-trifluoroethyl)- |
| | 1H-indol-2-yl]prop-2-yn-1-yl}amino)pyridin-2-yl]-2-methylpropanenitrile |
| 207. | 2-methyl-2-(5-{[3-(5-{[(1-methylazetidin-3-yl)amino]methyl}-1-(2,2,2-trifluoroethyl)- |
| | 1H-indol-2-yl)prop-2-yn-1-yl]amino}pyridin-2-yl)propanenitrile |
| 208. | 2-methyl-2-(5-{[3-(5-{[(oxetan-3-yl)amino]methyl}-1-(2,2,2-trifluoroethyl)-1H-indol-2- |
| | yl)prop-2-yn-1-yl]amino}pyridin-2-yl)propanenitrile |
| 209. | 2-(5-{[3-(5-{[4-(dimethylamino)-piperidin-1-yl]methyl}-1-(2,2,2-trifluoroethyl)-1H- |
| | indol-2-yl)prop-2-yn-1-yl]amino}pyridin-2-yl)-2-methylpropanenitrile |
| 210. | 2-methyl-2-{5-[(3-{5-[({1-[2-(4-methylpiperazin-1-yl)acetyl]piperidin-4- |
| | yl}amino)methyl]-1-(2,2,2-trifluoroethyl)-1H-indol-2-yl}prop-2-yn-1-yl)amino]pyridin- |
| | 2-yl}propanenitrile |
| 211. | 2-(5-{[3-(5-{[(1-methoxypropan-2-yl)amino]methyl}-1-(2,2,2-trifluoroethyl)-1H-indol-2- |
| | yl)prop-2-yn-1-yl]amino}pyridin-2-yl)-2-methylpropanenitrile |
| 212. | 2-methyl-2-(5-{[3-(5-{[(pyridin-4-ylmethyl)amino]methyl}-1-(2,2,2-trifluoroethyl)-1H- |
| | indol-2-yl)prop-2-yn-1-yl]amino}pyridin-2-yl)propanenitrile |
| 213. | 2-methyl-2-(5-{[3-(5-{[(pyridin-3-ylmethyl)amino]methyl}-1-(2,2,2-trifluoroethyl)-1H- |
| | indol-2-yl)prop-2-yn-1-yl]amino}pyridin-2-yl)propanenitrile |
| 214. | 2-[5-({3-[5-({[1-(dimethylamino)-propan-2-yl]amino}methyl)-1-(2,2,2-trifluoroethyl)- |
| | 1H-indol-2-yl]prop-2-yn-1-yl}amino)pyridin-2-yl]-2-methylpropanenitrile |
| 215. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(oxan-4-yl)acetamide |
| 216. | 2-[5-({3-[5-({[1-(2-methoxyacetyl)-piperidin-4-yl]amino}methyl)-1-(2,2,2- |
| | trifluoroethyl)-1H-indol-2-yl]prop-2-yn-1-yl}amino)pyridin-2-yl]-2-methylpropanenitrile |
| 217. | 2-methyl-2-{5-[(3-{5-[({1-[2-(oxan-4-yl)acetyl]piperidin-4-yl}amino)-methyl]-1-(2,2,2- |
| | trifluoroethyl)-1H-indol-2-yl}prop-2-yn-1-yl)amino]-pyridin-2-yl}propanenitrile |
| 218. | 2-methyl-2-{5-[(3-{5-[({1-[2-(pyridin-3-yl)acetyl]piperidin-4-yl}amino)-methyl]-1- |
| | (2,2,2-trifluoroethyl)-1H-indol-2-yl}prop-2-yn-1-yl)amino]-pyridin-2-yl}propanenitrile |
| 219. | 2-methyl-2-(5-{[3-(5-{[(1-{2-[(oxan-4-yl)amino]acetyl}piperidin-4-yl)amino]methyl}-1- |
| | (2,2,2-trifluoroethyl)-1H-indol-2-yl)prop-2-yn-1-yl]amino}pyridin-2-yl)-propanenitrile |
| 220. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-methyl-N-(propan-2- |
| | yl)acetamide |
| 221. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(2-methoxyethyl)-N- |
| | methylacetamide |
| 222. | 6-methanesulfonyl-N-[3-(5-{[(oxan-4-yl)amino]methyl}-1-(2,2,2-trifluoroethyl)-1H- |
| | indol-2-yl)prop-2-yn-1-yl]pyridin-3-amine |
| 223. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N,N-dimethylacetamide |
| 224. | 2-methyl-2-{5-[(3-{5-[({1-[2-oxo-2-(pyrrolidin-1-yl)ethyl]piperidin-4-yl}amino)methyl]- |
| | 1-(2,2,2-trifluoroethyl)-1H-indol-2-yl}prop-2-yn-1-yl)amino]pyridin-2-yl}propanenitrile |
| 225. | 4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)-N,N-dimethylpiperidine-1-carboxamide |
| 226. | 2-{5-[(3-{5-[({1-[2-(azetidin-1-yl)-2-oxoethyl]piperidin-4-yl}amino)-methyl]-1-(2,2,2- |
| | trifluoroethyl)-1H-indol-2-yl}prop-2-yn-1-yl)amino]-pyridin-2-yl}-2- |
| | methylpropanenitrile |
| 227. | 2-methyl-2-{5-[(3-{5-[({1-[2-(pyrrolidin-1-yl)acetyl]piperidin-4-yl}amino)methyl]-1- |
| | (2,2,2-trifluoroethyl)-1H-indol-2-yl}prop-2-yn-1-yl)amino]pyridin-2-yl}-propanenitrile |
| 228. | 2-(5-{[3-(5-{[(1-{2-[4-(dimethylamino)piperidin-1-yl]acetyl}piperidin-4-yl)amino]- |
| | methyl}-1-(2,2,2-trifluoroethyl)-1H-indol-2-yl)prop-2-yn-1-yl]amino}pyridin-2-yl)-2- |
| | methylpropanenitrile |
| 229. | 2-{5-[(3-{5-[({1-[2-(diethylamino)acetyl]piperidin-4-yl}amino)methyl]-1-(2,2,2- |
| | trifluoroethyl)-1H-indol-2-yl}prop-2-yn-1-yl)amino]pyridin-2-yl}-2-methylpropanenitrile |
| 230. | 2-methyl-2-(5-{[3-(5-{[(1-{2-[methyl(propan-2-yl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)propanenitrile |
| 231. | 2-methyl-2-{5-[(3-{5-[({1-[2-(pyridin-4-yl)acetyl]piperidin-4-yl}amino)methyl]-1-(2,2,2- |
| | trifluoroethyl)-1H-indol-2-yl}prop-2-yn-1-yl)amino]pyridin-2-yl}propanenitrile |
| 232. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(pyridin-4-yl)acetamide |
| 233. | 2-methyl-2-{5-[(3-{5-[({1-[2-(morpholin-4-yl)-2-oxoethyl]piperidin-4-yl}amino)methyl]- |
| | 1-(2,2,2-trifluoroethyl)-1H-indol-2-yl}prop-2-yn-1-yl)amino]pyridin-2-yl}propanenitrile |
| 234. | 2-methyl-2-{5-[(3-{5-[({1-[2-(4-methylpiperazin-1-yl)-2-oxoethyl]piperidin-4- |
| | yl}amino)methyl]-1-(2,2,2-trifluoroethyl)-1H-indol-2-yl}prop-2-yn-1-yl)amino]pyridin- |
| | 2-yl}propanenitrile |
| 235. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(pyridin-3-yl)acetamide |
| 236. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(1-methylpiperidin-4- |
| | yl)acetamide |
| 237. | 2-methyl-2-[5-({3-[5-({[4-(morpholin-4-yl)cyclohexyl]amino}methyl)-1-(2,2,2- |
| | trifluoroethyl)-1H-indol-2-yl]prop-2-yn-1-yl}amino)pyridin-2-yl]propanenitrile |
| 238. | 2-{5-[(3-{5-[({1-[2-(4-hydroxypiperidin-1-yl)acetyl]piperidin-4-yl}amino)methyl]-1- |
| | (2,2,2-trifluoroethyl)-1H-indol-2-yl}prop-2-yn-1-yl)amino]pyridin-2-yl}-2- |
| | methylpropanenitrile |
| 239. | 2-{5-[(3-{5-[({1-[2-(4-acetylpiperazin-1-yl)acetyl]piperidin-4-yl}amino)methyl]-1-(2,2,2- |
| | trifluoroethyl)-1H-indol-2-yl}prop-2-yn-1-yl)amino]pyridin-2-yl}-2-methylpropanenitrile |
| 240. | 2-(5-{[3-(5-{[(1,1-dioxo-1λ⁶-thian-4-yl)amino]methyl}-1-(2,2,2-trifluoroethyl)-1H-indol- |
| | 2-yl)prop-2-yn-1-yl]amino}pyridin-2-yl)-2-methylpropanenitrile |
| 241. | 2-{5-[(3-{5-[({1-[2-(1,1-dioxo-1λ⁶,4-thiomorpholin-4-yl)acetyl]piperidin-4- |
| | yl}amino)methyl]-1-(2,2,2-trifluoroethyl)-1H-indol-2-yl}prop-2-yn-1-yl)amino]pyridin- |
| | 2-yl}-2-methylpropanenitrile |
| 242. | 2-[5-({3-[5-({[1-(4-acetylpiperazine-1-carbonyl)piperidin-4-yl]amino}methyl)-1-(2,2,2- |
| | trifluoroethyl)-1H-indol-2-yl]prop-2-yn-1-yl}amino)pyridin-2-yl]-2-methylpropanenitrile |
| 243. | 2-(5-{[3-(5-{[(1-{2-[bis(2-hydroxyethyl)amino]acetyl}piperidin-4-yl)amino]methyl}-1- |
| | (2,2,2-trifluoroethyl)-1H-indol-2-yl)prop-2-yn-1-yl]amino}pyridin-2-yl)-2- |
| | methylpropanenitrile |
| 244. | 2-methyl-2-{5-[(3-{5-[({1-[2-(3-oxopiperazin-1-yl)acetyl]piperidin-4-yl}amino)methyl]- |
| | 1-(2,2,2-trifluoroethyl)-1H-indol-2-yl}prop-2-yn-1-yl)amino]pyridin-2-yl}propanenitrile |
| 245. | 2-methyl-2-[5-({3-[5-({[1-(morpholine-4-carbonyl)piperidin-4-yl]amino}-methyl)-1- |
| | (2,2,2-trifluoroethyl)-1H-indol-2-yl]prop-2-yn-1-yl}amino)pyridin-2-yl]propanenitrile |
| 246. | 2-methyl-2-(5-{[3-(5-{[(1-methylpiperidin-3-yl)amino]methyl}-1-(2,2,2-trifluoroethyl)- |
| | 1H-indol-2-yl)prop-2-yn-1-yl]amino}pyridin-2-yl)propanenitrile |
| 247. | N-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)-cyclohexyl]acetamide |
| 248. | 2-{5-[(3-{5-[({1-[2-(1H-imidazol-1-yl)acetyl]piperidin-4-yl}amino)-methyl]-1-(2,2,2- |
| | trifluoroethyl)-1H-indol-2-yl}prop-2-yn-1-yl)amino]pyridin-2-yl}-2-methylpropanenitrile |
| 249. | 2-(5-{[3-(5-{[(1-{2-[(2-methoxyethyl)(methyl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)-2-methylpropanenitrile |
| 250. | N-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)-cyclohexyl]methanesulfonamide |
| 251. | 2-methyl-2-(5-{[3-(5-{[(1-methyl-6-oxopiperidin-3-yl)amino]methyl}-1-(2,2,2- |
| | trifluoroethyl)-1H-indol-2-yl)prop-2-yn-1-yl]amino}pyridin-2-yl)propanenitrile |
| 252. | 2-[5-({3-[5-({[3-(dimethylamino)cyclohexyl]amino}methyl)-1-(2,2,2-trifluoroethyl)-1H- |
| | indol-2-yl]prop-2-yn-1-yl}amino)pyridin-2-yl]-2-methylpropanenitrile |
| 253. | 2-methyl-2-[5-({3-[5-({[1-(4-methylpiperazine-1-carbonyl)piperidin-4-yl]amino}methyl)- |
| | 1-(2,2,2-trifluoroethyl)-1H-indol-2-yl]prop-2-yn-1-yl}amino)pyridin-2-yl]propanenitrile |
| 254. | 2-{5-[(3-{5-[({1-[4-(dimethylamino)piperidine-1-carbonyl]piperidin-4-yl}amino)- |
| | methyl]-1-(2,2,2-trifluoroethyl)-1H-indol-2-yl}prop-2-yn-1-yl)amino]pyridin-2-yl}-2- |
| | methylpropanenitrile |
| 255. | 2-{5-[(3-{5-[({1-[2-(3-hydroxypyrrolidin-1-yl)acetyl]-piperidin-4-yl}amino)methyl]-1- |
| | (2,2,2-trifluoroethyl)-1H-indol-2-yl}prop-2-yn-1-yl)amino]pyridin-2-yl}-2- |
| | methylpropanenitrile |
| 256. | 2-{5-[(3-{5-[({1-[2-(3-methoxypyrrolidin-1-yl)acetyl]-piperidin-4-yl}amino)methyl]-1- |
| | (2,2,2-trifluoroethyl)-1H-indol-2-yl}prop-2-yn-1-yl)amino]pyridin-2-yl}-2- |
| | methylpropanenitrile |
| 257. | 2-methyl-2-[5-({3-[5-({[1-(2-{2-oxa-8-azaspiro[4.5]decan-8-yl}acetyl)-piperidin-4- |
| | yl]amino}methyl)-1-(2,2,2-trifluoroethyl)-1H-indol-2-yl]prop-2-yn-1-yl}amino)pyridin- |
| | 2-yl]propanenitrile |
| 258. | 2-{5-[(3-{5-[({1-[2-(4-hydroxy-4-methylpiperidin-1-yl)acetyl]piperidin-4- |
| | yl}amino)methyl]-1-(2,2,2-trifluoroethyl)-1H-indol-2-yl}prop-2-yn-1-yl)amino]pyridin- |
| | 2-yl}-2-methylpropanenitrile |
| 259. | 2-(5-{[3-(5-{[(1-{2-[bis(2-methoxyethyl)amino]acetyl}piperidin-4-yl)amino]methyl}-1- |
| | (2,2,2-trifluoroethyl)-1H-indol-2-yl)prop-2-yn-1-yl]amino}pyridin-2-yl)-2- |
| | methylpropanenitrile |
| 260. | 2-(5-{[3-(5-{[(1-{2-[methoxy(methyl)amino]acetyl}piperidin-4-yl)amino]methyl}-1- |
| | (2,2,2-trifluoroethyl)-1H-indol-2-yl)prop-2-yn-1-yl]amino}pyridin-2-yl)-2- |
| | methylpropanenitrile |
| 261. | 2-(5-{[3-(5-{[(1-{2-[(2,3-dihydroxypropyl)(methyl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)-2-methylpropanenitrile |
| 262. | 2-methyl-2-(5-{[3-(5-{[(1-methyl-2-oxopiperidin-4-yl)amino]methyl}-1-(2,2,2- |
| | trifluoroethyl)-1H-indol-2-yl)prop-2-yn-1-yl]amino}pyridin-2-yl)propanenitrile |
| 263. | 2-methyl-2-(5-{[3-(5-{[(1-{2-[methyl(1-methylpiperidin-4-yl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)propanenitrile |
| 264. | 2-methyl-2-[5-({3-[5-({[1-(2-{9-methyl-3,9-diazaspiro[5.5]undecan-3- |
| | yl}acetyl)piperidin-4-yl]amino}methyl)-1-(2,2,2-trifluoroethyl)-1H-indol-2-yl]prop-2-yn- |
| | 1-yl}amino)pyridin-2-yl]propanenitrile |
| 265. | 2-(5-{[3-(5-{[(1-{2-[3-(dimethyl-amino)pyrrolidin-1-yl]acetyl}piperidin-4- |
| | yl)amino]methyl}-1-(2,2,2-trifluoroethyl)-1H-indol-2-yl)prop-2-yn-1-yl]amino}pyridin- |
| | 2-yl)-2-methylpropanenitrile |
| 266. | N-[3-(5-{[(oxan-4-yl)amino]methyl}-1-(2,2,2-trifluoroethyl)-1H-indol-2-yl)prop-2-yn-1- |
| | yl]-6-(pyrrolidine-1-carbonyl)pyridin-3-amine |
| 267. | 6-(morpholine-4-carbonyl)-N-[3-(5-{[(oxan-4-yl)amino]methyl}-1-(2,2,2-trifluoroethyl)- |
| | 1H-indol-2-yl)prop-2-yn-1-yl]pyridin-3-amine |
| 268. | 2-chloro-N-[3-(5-{[(oxan-4-yl)amino]-methyl}-1-(2,2,2-trifluoroethyl)-1H-indol-2- |
| | yl)prop-2-yn-1-yl]pyrimidin-5-amine |
| 269. | 5-{[3-(5-{[(oxan-4-yl)amino]methyl}-1-(2,2,2-trifluoroethyl)-1H-indol-2-yl)prop-2-yn-1- |
| | yl]amino}-N-phenylpyridine-2-carboxamide |
| 270. | N-methyl-5-{[3-(5-([(oxan-4-yl)amino]methyl}-1-(2,2,2-trifluoroethyl)-1H-indol-2- |
| | yl)prop-2-yn-1-yl]amino]-N-(propan-2-yl)pyridine-2-carboxamide |
| 271. | 5-{[3-(5-{[(oxan-4-yl)amino]methyl}-1-(2,2,2-trifluoroethyl)-1H-indol-2-yl)prop-2-yn-1- |
| | yl]amino}-N-(pyridin-4-yl)pyridine-2-carboxamide |
| 272. | 5-{[3-(5-{[(oxan-4-yl)amino]methyl}-1-(2,2,2-trifluoroethyl)-1H-indol-2-yl)prop-2-yn-1- |
| | yl]amino}-N-(pyridin-3-yl)pyridine-2-carboxamide |
| 273. | N-(1-methylazetidin-3-yl)-5-{[3-(5-{[(oxan-4-yl)amino]methyl}-1-(2,2,2-trifluoroethyl)- |
| | 1H-indol-2-yl)prop-2-yn-1-yl]amino}pyridine-2-carboxamide |
| 274. | N,N-diethyl-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 275. | 5-{[3-(5-{[(oxan-4-yl)amino]methyl}-1-(2,2,2-trifluoroethyl)-1H-indol-2-yl)prop-2-yn-1- |
| | yl]amino}-N-(oxetan-3-yl)pyridine-2-carboxamide |
| 276. | 1-(4-{[(2-{3-[(2-tert-butylpyrimidin-5-yl)amino]prop-1-yn-1-yl}-1-(2,2,2-trifluoroethyl)- |
| | 1H-indol-5-yl)methyl]amino}piperidin-1-yl)-2-(dimethylamino)ethan-1-one |
| 277. | 1-(4-{[(2-{3-[(6-chloropyridin-3-yl)amino]prop-1-yn-1-yl}-1-(2,2,2-trifluoroethyl)-1H- |
| | indol-5-yl)methyl]amino}piperidin-1-yl)-2-(dimethylamino)ethan-1-one |
| 278. | 5-[(3-{5-[({1-[2-(dimethylamino)-acetyl]piperidin-4-yl}amino)methyl]-1-(2,2,2- |
| | trifluoroethyl)-1H-indol-2-yl}prop-2-yn-1-yl)amino]-N-(1-methylpiperidin-4-yl)pyridine- |
| | 2-carboxamide |
| 279. | 1-(4-{[(2-{3-[(4-chloro-3-fluorophenyl)amino]prop-1-yn-1-yl}-1-(2,2,2-trifluoroethyl)- |
| | 1H-indol-5-yl)methyl]amino}piperidin-1-yl)-2-(dimethylamino)ethan-1-one |
| 280. | 2-(dimethylamino)-1-(4-{[(2-{3-[(6-methylpyridin-3-yl)amino]prop-1-yn-1-yl}-1-(2,2,2- |
| | trifluoroethyl)-1H-indol-5-yl)methyl]amino}piperidin-1-yl)ethan-1-one |
| 281. | 1-(4-{[(2-{3-[(6-tert-butylpyridin-3-yl)amino]prop-1-yn-1-yl}-1-(2,2,2-trifluoroethyl)- |
| | 1H-indol-5-yl)methyl]amino}piperidin-1-yl)-2-(dimethylamino)ethan-1-one |
| 282. | N-[6-(1-cyano-1-methylethyl)pyridin-3-yl]-3-[1-(2-fluoroethyl)-5-{[(1-methylpiperidin-4- |
| | yl)amino]methyl}-1H-indol-2-yl]prop-2-ynamide |
| 283. | 2-{5-[(3-{5-[({1-[2-(dimethylamino)acetyl]piperidin-4-yl}amino)methyl]-1-ethyl-1H- |
| | indol-2-yl}prop-2-yn-1-yl)amino]pyridin-2-yl}-2-methylpropanenitrile |
| 284. | 2-[5-({3-[1-(2,2-difluoroethyl)-5-[({1-[2-(dimethylamino)acetyl]piperidin-4- |
| | yl}amino)methyl]-1H-indol-2-yl]prop-2-yn-1-yl}amino)pyridin-2-yl]-2- |
| | methylpropanenitrile |
| 285. | 2-{5-[(3-{5-[({1-[2-(dimethylamino)acetyl]piperidin-4-yl}amino)methyl]-1-(2- |
| | fluoroethyl)-1H-indol-2-yl}prop-2-yn-1-yl)amino]pyridin-2-yl}-2-methylpropanenitrile |
| 286. | N-(6-chloropyridin-3-yl)-3-(5-{[(1-methylpiperidin-4-yl)amino]methyl}-1-(2,2,2- |
| | trifluoroethyl)-1H-indol-2-yl)prop-2-ynamide |
| 287. | 2-(5-{[3-(5-{[(1-acetylpiperidin-4-yl)amino]methyl}-1-(oxiran-2-ylmethyl)-1H-indol-2- |
| | yl)prop-2-yn-1-yl]amino}pyridin-2-yl)-2-methylpropanenitrile |
| 288. | 2-methyl-2-{5-[(3-{5-[(oxan-4-yl)amino]-1-(2,2,2-trifluoroethyl)-1H-indol-2-yl}prop-2- |
| | yn-1-yl)amino]pyridin-2-yl}propanenitrile |
| 289. | 2-{5-[(3-{4-[(1-acetylpiperidin-4-yl)amino]-1-(2,2,2-trifluoroethyl)-1H-indol-2-yl}prop- |
| | 2-yn-1-yl)amino]pyridin-2-yl}-2-methylpropanenitrile |
| 290. | 2-methyl-2-{5-[(3-{4-[(propan-2-yl)amino]-1-(2,2,2-trifluoroethyl)-1H-indol-2-yl}prop- |
| | 2-yn-1-yl)amino]pyridin-2-yl}propanenitrile |
| 291. | 2-methyl-2-{5-[(3-{4-[(piperidin-4-yl)amino]-1-(2,2,2-trifluoroethyl)-1H-indol-2- |
| | yl}prop-2-yn-1-yl)amino]pyridin-2-yl}propanenitrile |
| 292. | 2-(5-{[3-(4-{[1-(2-methoxyethyl)-piperidin-4-yl]amino}-1-(2,2,2-trifluoroethyl)-1H- |
| | indol-2-yl)prop-2-yn-1-yl]amino}pyridin-2-yl)-2-methylpropanenitrile |
| 293. | 2-{5-[(3-{4-[(1,1-dioxo-1λ⁶-thian-4-yl)amino]-1-(2,2,2-trifluoroethyl)-1H-indol-2- |
| | yl}prop-2-yn-1-yl)amino]pyridin-2-yl}-2-methylpropanenitrile |
| 294. | 3-[2-(3-{[6-(1-cyano-1-methylethyl)-pyridin-3-yl]amino}prop-1-yn-1-yl)-1-(2,2,2- |
| | trifluoroethyl)-1H-indol-4-yl]-1-(oxan-4-yl)urea |
| 295. | 3-[2-(3-{[6-(1-cyano-1-methylethyl)-pyridin-3-yl]amino}prop-1-yn-1-yl)-1-(2,2,2- |
| | trifluoroethyl)-1H-indol-4-yl]-1-(1-methylpiperidin-4-yl)urea |
| 296. | 2-(4-{[2-(3-{[6-(1-cyano-1-methylethyl)-pyridin-3-yl]amino}prop-1-yn-1-yl)-1-(2,2,2- |
| | trifluoroethyl)-1H-indol-4-yl]amino}piperidin-1-yl)-N,N-dimethylacetamide |
| 297. | 2-methyl-2-(5-{[3-(4-{[1-(propan-2-yl)piperidin-4-yl]amino}-1-(2,2,2-trifluoroethyl)-1H- |
| | indol-2-yl)prop-2-yn-1-yl]amino}pyridin-2-yl)propanenitrile |
| 298. | 2-methyl-2-(5-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}pyridin-2-yl)propanenitrile |
| 299. | 4-{[2-(3-{[6-(1-cyano-1-methylethyl)-pyridin-3-yl]amino}prop-1-yn-1-yl)-1-(2,2,2- |
| | trifluoroethyl)-1H-indol-4-yl]amino}-N,N-dimethylpiperidine-1-carboxamide |
| 300. | N-[2-(3-{[6-(1-cyano-1-methylethyl)-pyridin-3-yl]amino}prop-1-yn-1-yl)-1-(2,2,2- |
| | trifluoroethyl)-1H-indol-4-yl]-4-methylpiperazine-1-carboxamide |
| 301. | 1-[2-(3-{[6-(1-cyano-1-methylethyl)pyridin-3-yl]amino}prop-1-yn-1-yl)-1-(2,2,2- |
| | trifluoroethyl)-1H-indol-4-yl]-3,3-dimethylurea |
| 302. | N-[2-(3-{[6-(1-cyano-1-methylethyl)-pyridin-3-yl]amino}prop-1-yn-1-yl)-1-(2,2,2- |
| | trifluoroethyl)-1H-indol-4-yl]morpholine-4-carboxamide |
| 303. | 2-{5-[(3-{4-[(4-hydroxycyclohexyl)-amino]-1-(2,2,2-trifluoroethyl)-1H-indol-2-yl}prop- |
| | 2-yn-1-yl)amino]pyridin-2-yl}-2-methylpropanenitrile |
| 304. | 2-methyl-2-[5-({3-[4-({1-[2-(4-methylpiperazin-1-yl)-2-oxoethyl]-piperidin-4-yl}amino)- |
| | 1-(2,2,2-trifluoroethyl)-1H-indol-2-yl]prop-2-yn-1-yl}amino)pyridin-2-yl]propanenitrile |
| 305. | 2-methyl-2-{5-[(3-{4-[(oxan-4-ylmethyl)amino]-1-(2,2,2-trifluoroethyl)-1H-indol-2- |
| | yl}prop-2-yn-1-yl)amino]pyridin-2-yl}propanenitrile |
| 306. | 2-{5-[(3-{4-[(1-ethylpiperidin-4-yl)amino]-1-(2,2,2-trifluoroethyl)-1H-indol-2-yl}prop-2- |
| | yn-1-yl)amino]pyridin-2-yl}-2-methylpropanenitrile |
| 307. | 2-(5-{[3-(4-{[1-(2-hydroxyethyl)piperidin-4-yl]amino}-1-(2,2,2-trifluoroethyl)-1H-indol- |
| | 2-yl)prop-2-yn-1-yl]amino}pyridin-2-yl)-2-methylpropanenitrile |
| 308. | 2-{5-[(3-{4-[(1-methanesulfonylpiperidin-4-yl)amino]-1-(2,2,2-trifluoroethyl)-1H-indol- |
| | 2-yl}prop-2-yn-1-yl)amino]pyridin-2-yl}-2-methylpropanenitrile |
| 309. | 2-(5-{[3-(4-{[1-(2-methanesulfonyl-ethyl)piperidin-4-yl]amino}-1-(2,2,2-trifluoroethyl)- |
| | 1H-indol-2-yl)prop-2-yn-1-yl]amino}pyridin-2-yl)-2-methylpropanenitrile |
| 310. | 2-methyl-2-(5-{[3-(4-{[(1R,4R)-4-hydroxycyclohexyl]amino]-1-(2,2,2-trifluoroethyl)- |
| | 1H-indol-2-yl)prop-2-yn-1-yl]amino}pyridin-2-yl)propanenitrile |
| 311. | 2-methyl-2-(5-{[3-(4-{[(1S,4S)-4-hydroxycyclohexyl]amino]-1-(2,2,2-trifluoroethyl)-1H- |
| | indol-2-yl)prop-2-yn-1-yl]amino}pyridin-2-yl)propanenitrile |
| 312. | 2-methyl-2-[5-({3-[4-({1-[2-(morpholin-4-yl)-2-oxoethyl]piperidin-4-yl}amino)-1-(2,2,2- |
| | trifluoroethyl)-1H-indol-2-yl]prop-2-yn-1-yl}amino)pyridin-2-yl]propanenitrile |
| 313. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)-N,N-bis(2-methoxyethyl)acetamide |
| 314. | 2-methyl-2-{5-[(3-{4-[(pyrrolidin-3-yl)amino]-1-(2,2,2-trifluoroethyl)-1H-indol-2- |
| | yl}prop-2-yn-1-yl)amino]pyridin-2-yl}propanenitrile |
| 315. | 2-methyl-2-{5-[(3-{4-[(1-methylpyrrolidin-3-yl)amino]-1-(2,2,2-trifluoroethyl)-1H-indol- |
| | 2-yl}prop-2-yn-1-yl)amino]pyridin-2-yl}propanenitrile |
| 316. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)acetamide |
| 317. | methyl 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]amino}piperidin-1-yl)acetate |
| 318. | 2-[5-({3-[4-({1-[2-(4-hydroxypiperidin-1-yl)-2-oxoethyl]piperidin-4-yl}amino)-1-(2,2,2- |
| | trifluoroethyl)-1H-indol-2-yl]prop-2-yn-1-yl}amino)pyridin-2-yl]-2-methylpropanenitrile |
| 319. | 2-methyl-2-{5-[(3-{4-[(2-methylpiperidin-4-yl)amino]-1-(2,2,2-trifluoroethyl)-1H-indol- |
| | 2-yl}prop-2-yn-1-yl)amino]pyridin-2-yl}propanenitrile |
| 320. | 2-{5-[(3-{4-[(1,1-dioxo-1λ⁶-thiolan-3-yl)amino]-1-(2,2,2-trifluoroethyl)-1H-indol-2- |
| | yl}prop-2-yn-1-yl)amino]pyridin-2-yl}-2-methylpropanenitrile |
| 321. | 2-methyl-2-[5-({3-[4-({1-[2-oxo-2-(pyrrolidin-1-yl)ethyl]piperidin-4-yl}amino)-1-(2,2,2- |
| | trifluoroethyl)-1H-indol-2-yl]prop-2-yn-1-yl}amino)pyridin-2-yl]propanenitrile |
| 322. | 2-{5-[(3-{4-[(1-{2-[4-(dimethylamino)piperidin-1-yl]-2-oxoethyl}piperidin-4-yl)amino]- |
| | 1-(2,2,2-trifluoroethyl)-1H-indol-2-yl}prop-2-yn-1-yl)amino]pyridin-2-yl}-2- |
| | methylpropanenitrile |
| 323. | 2-[5-({3-[4-({1-[2-(dimethylamino)acetyl]piperidin-4-yl}amino)-1-(2,2,2-trifluoroethyl)- |
| | 1H-indol-2-yl]prop-2-yn-1-yl}amino)pyridin-2-yl]-2-methylpropanenitrile |
| 324. | 2-(5-{[3-(4-{[1-(1,1-dioxo-1λ⁶-thian-4-yl)piperidin-4-yl]amino}-1-(2,2,2-trifluoroethyl)- |
| | 1H-indol-2-yl)prop-2-yn-1-yl]amino}pyridin-2-yl)-2-methylpropanenitrile |
| 325. | 2-(5-{[3-(4-{[1-(cyanomethyl)piperidin-4-yl]amino}-1-(2,2,2-trifluoroethyl)-1H-indol-2- |
| | yl)prop-2-yn-1-yl]amino}pyridin-2-yl)-2-methylpropanenitrile |
| 326. | 2-methyl-2-[5-({3-[1-(2,2,2-trifluoroethyl)-4-{[1-(2,2,2-trifluoroethyl)piperidin-4- |
| | yl]amino}-1H-indol-2-yl]prop-2-yn-1-yl}amino)pyridin-2-yl]propanenitrile |
| 327. | 2-{5-[(3-{4-[(1-{2-[4-(2-methanesulfonylethyl)piperazin-1-yl]-2-oxoethyl}piperidin-4- |
| | yl)amino]-1-(2,2,2-trifluoroethyl)-1H-indol-2-yl}prop-2-yn-1-yl)amino]pyridin-2-yl}-2- |
| | methylpropanenitrile |
| 328. | 2-[5-({3-[4-({1-[2-(1,1-dioxo-1λ⁶,4-thiomorpholin-4-yl)-2-oxoethyl]piperidin-4- |
| | yl}amino)-1-(2,2,2-trifluoroethyl)-1H-indol-2-yl]prop-2-yn-1-yl}amino)pyridin-2-yl]-2- |
| | methylpropanenitrile |
| 329. | 2-(5-{[3-(4-{[1-(1-methanesulfonylpiperidin-4-yl)piperidin-4-yl]amino}-1-(2,2,2- |
| | trifluoroethyl)-1H-indol-2-yl)prop-2-yn-1-yl]amino}pyridin-2-yl)-2-methylpropanenitrile |
| 330. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)-N-(2,3-dihydroxypropyl)-N- |
| | methylacetamide |
| 331. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)-N-(2,3-dihydroxypropyl)acetamide |
| 332. | 2-[5-({3-[4-({1-[2-(4-methanesulfonylpiperazin-1-yl)-2-oxoethyl]piperidin-4-yl}amino)- |
| | 1-(2,2,2-trifluoroethyl)-1H-indol-2-yl]prop-2-yn-1-yl}amino)pyridin-2-yl]-2- |
| | methylpropanenitrile |
| 333. | 2-{5-[(3-{4-[(1-{2-[4-(2-hydroxyethyl)piperazin-1-yl]-2-oxoethyl}piperidin-4-yl)amino]- |
| | 1-(2,2,2-trifluoroethyl)-1H-indol-2-yl}prop-2-yn-1-yl)amino]pyridin-2-yl}-2- |
| | methylpropanenitrile |
| 334. | 2-methyl-2-(5-{[3-(4-{[1-(oxan-4-yl)piperidin-4-yl]amino}-1-(2,2,2-trifluoroethyl)-1H- |
| | indol-2-yl)prop-2-yn-1-yl]amino}pyridin-2-yl)propanenitrile |
| 335. | 2-[5-({3-[4-({1-[1-(2-methanesulfonylethyl)piperidin-4-yl]piperidin-4-yl}amino)-1- |
| | (2,2,2-trifluoroethyl)-1H-indol-2-yl]prop-2-yn-1-yl}amino)pyridin-2-yl]-2- |
| | methylpropanenitrile |
| 336. | 2-[5-({3-[4-({1-[1-(2-methoxyethyl)piperidin-4-yl]piperidin-4-yl}amino)-1-(2,2,2- |
| | trifluoroethyl)-1H-indol-2-yl]prop-2-yn-1-yl}amino)pyridin-2-yl]-2-methylpropanenitrile |
| 337. | 2-[5-({3-[4-({1-[1-(2-hydroxyethyl)piperidin-4-yl]piperidin-4-yl}amino)-1-(2,2,2- |
| | trifluoroethyl)-1H-indol-2-yl]prop-2-yn-1-yl}amino)pyridin-2-yl]-2-methylpropanenitrile |
| 338. | 2-[5-({3-[4-({1-[2-(dimethylamino)ethyl]piperidin-4-yl}amino)-1-(2,2,2-trifluoroethyl)- |
| | 1H-indol-2-yl]prop-2-yn-1-yl}amino)pyridin-2-yl]-2-methylpropanenitrile |
| 339. | 2-(5-{[3-(4-{[1-(1-acetylpiperidin-4-yl)piperidin-4-yl]amino}-1-(2,2,2-trifluoroethyl)-1H- |
| | indol-2-yl)prop-2-yn-1-yl]amino}pyridin-2-yl)-2-methylpropanenitrile |
| 340. | 2-methyl-2-[5-({3-[4-({1-[(1R,4R)-4-hydroxycyclohexyl]piperidin-4-yl}amino)-1-(2,2,2- |
| | trifluoroethyl)-1H-indol-2-yl]prop-2-yn-1-yl}amino)pyridin-2-yl]propanenitrile |
| 341. | 2-methyl-2-[5-({3-[4-({1-[(1S,4S)-4-hydroxycyclohexyl]piperidin-4-yl}amino)-1-(2,2,2- |
| | trifluoroethyl)-1H-indol-2-yl]prop-2-yn-1-yl}amino)pyridin-2-yl]propanenitrile |
| 342. | N-(1-methylpiperidin-4-yl)-2-{3-[(6-methylpyridin-3-yl)amino]prop-1-yn-1-yl}-1-(2,2,2- |
| | trifluoroethyl)-1H-indol-4-amine |
| 343. | 2-methyl-2-{5-[(3-{4-[(1-methylpiperidin-4-yl)amino]-1-(2,2,2-trifluoroethyl)-1H-indol- |
| | 2-yl}prop-2-yn-1-yl)amino]pyridin-2-yl}propanenitrile |
| 344. | 5-[(3-{4-[(1-methylpiperidin-4-yl)amino]-1-(2,2,2-trifluoroethyl)-1H-indol-2-yl}prop-2- |
| | yn-1-yl)amino]-N-(pyridin-3-yl)pyridine-2-carboxamide |
| 345. | 5-[(3-{4-[(1-methylpiperidin-4-yl)amino]-1-(2,2,2-trifluoroethyl)-1H-indol-2-yl}prop-2- |
| | yn-1-yl)amino]pyridine-2-carbonitrile |
| 346. | N-[6-(1-cyano-1-methylethyl)pyridin-3-yl]-3-{4-[(1-methylpiperidin-4-yl)amino]-1- |
| | (2,2,2-trifluoroethyl)-1H-indol-2-yl}prop-2-ynamide |
| 347. | 2-{3-[(2-fluorophenyl)amino]prop-1-yn-1-yl}-N-(1-methylpiperidin-4-yl)-1-(2,2,2- |
| | trifluoroethyl)-1H-indol-4-amine |
| 348. | 2-{3-[(3-fluorophenyl)amino]prop-1-yn-1-yl}-N-(1-methylpiperidin-4-yl)-1-(2,2,2- |
| | trifluoroethyl)-1H-indol-4-amine |
| 349. | 4-amino-N-(3-{4-[(1-methylpiperidin-4-yl)amino]-1-(2,2,2-trifluoroethyl)-1H-indol-2- |
| | yl}prop-2-yn-1-yl)benzene-1-sulfonamide |
| 350. | 2-{3-[(6-tert-butylpyridin-3-yl)amino]prop-1-yn-1-yl}-N-(1-methylpiperidin-4-yl)-1- |
| | (2,2,2-trifluoroethyl)-1H-indol-4-amine |
| 351. | 2-{3-[(4-fluorophenyl)amino]prop-1-yn-1-yl}-N-(1-methylpiperidin-4-yl)-1-(2,2,2- |
| | trifluoroethyl)-1H-indol-4-amine |
| 352. | N,N-dimethyl-5-[(3-{4-[(1-methylpiperidin-4-yl)amino]-1-(2,2,2-trifluoroethyl)-1H- |
| | indol-2-yl}prop-2-yn-1-yl)amino]pyridine-2-carboxamide |
| 353. | 5-[(3-{4-[(1-methylpiperidin-4-yl)amino]-1-(2,2,2-trifluoroethyl)-1H-indol-2-yl}prop-2- |
| | yn-1-yl)amino]-N-(propan-2-yl)pyridine-2-carboxamide |
| 354. | N-(pyridin-3-yl)-5-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}pyridine-2-carboxamide |
| 355. | N-(pyridin-3-yl)-5-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}pyridine-2-carboxamide |
| 356. | 2-{3-[(6-methanesulfonylpyridin-3-yl)amino]prop-1-yn-1-yl}-N-(1-methylpiperidin-4- |
| | yl)-1-(2,2,2-trifluoroethyl)-1H-indol-4-amine |
| 357. | 6-tert-butyl-N-(3-{4-[(1-methylpiperidin-4-yl)amino]-1-(2,2,2-trifluoroethyl)-1H-indol-2- |
| | yl}prop-2-yn-1-yl)pyridine-3-carboxamide |
| 358. | 2-{3-[(6-chloropyridin-3-yl)amino]prop-1-yn-1-yl}-N-(1-methylpiperidin-4-yl)-1-(2,2,2- |
| | trifluoroethyl)-1H-indol-4-amine |
| 359. | 2-{4-[(3-{4-[(1-methylpiperidin-4-yl)amino]-1-(2,2,2-trifluoroethyl)-1H-indol-2-yl}prop- |
| | 2-yn-1-yl)amino]phenyl}propan-2-ol |
| 360. | 6-methyl-N-(3-{4-[(1-methylpiperidin-4-yl)amino]-1-(2,2,2-trifluoroethyl)-1H-indol-2- |
| | yl}prop-2-yn-1-yl)pyridine-3-carboxamide |
| 361. | N-(1-methylpiperidin-4-yl)-1-(2,2,2-trifluoroethyl)-2-(3-{[6-(trifluoromethyl)pyridin-3- |
| | yl]amino}prop-1-yn-1-yl)-1H-indol-4-amine |
| 362. | 3-(3-{4-[(1-methylpiperidin-4-yl)amino]-1-(2,2,2-trifluoroethyl)-1H-indol-2-yl}prop-2- |
| | yn-1-yl)-1-phenylurea |
| 363. | 2-{3-[(4-tert-butyl-2-fluorophenyl)-amino]prop-1-yn-1-yl}-N-(1-methylpiperidin-4-yl)-1- |
| | (2,2,2-trifluoroethyl)-1H-indol-4-amine |
| 364. | 2-{3-fluoro-4-[(3-{4-[(1-methylpiperidin-4-yl)amino]-1-(2,2,2-trifluoroethyl)-1H-indol-2- |
| | yl}prop-2-yn-1-yl)amino]phenyl}-2-methylpropanenitrile |
| 365. | 4-[(3-{4-[(1-methylpiperidin-4-yl)amino]-1-(2,2,2-trifluoroethyl)-1H-indol-2-yl}prop-2- |
| | yn-1-yl)amino]benzene-1-sulfonamide |
| 366. | 2-{3-[(2,6-difluoro-4-methane-sulfonylphenyl)amino]prop-1-yn-1-yl}-N-(1- |
| | methylpiperidin-4-yl)-1-(2,2,2-trifluoroethyl)-1H-indol-4-amine |
| 367. | N-methyl-4-[(3-{4-[(1-methylpiperidin-4-yl)amino]-1-(2,2,2-trifluoroethyl)-1H-indol-2- |
| | yl}prop-2-yn-1-yl)amino]benzene-1-sulfonamide |
| 368. | 2-{3-[(4-methanesulfonyl-3-methoxyphenyl)amino]prop-1-yn-1-yl}-N-(1- |
| | methylpiperidin-4-yl)-1-(2,2,2-trifluoroethyl)-1H-indol-4-amine |
| 369. | 2-{3-[(4-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 370. | 2-{3-[(5-methanesulfonylpyridin-2-yl)amino]prop-1-yn-1-yl}-N-(1-methylpiperidin-4- |
| | yl)-1-(2,2,2-trifluoroethyl)-1H-indol-4-amine |
| 371. | 2-{3-[(4-methanesulfonyl-2-methylphenyl)amino]prop-1-yn-1-yl}-N-(1-methylpiperidin- |
| | 4-yl)-1-(2,2,2-trifluoroethyl)-1H-indol-4-amine |
| 372. | methyl 3-methoxy-4-[(3-{4-[(1-methylpiperidin-4-yl)amino]-1-(2,2,2-trifluoroethyl)-1H- |
| | indol-2-yl}prop-2-yn-1-yl)amino]benzoate |
| 373. | N-{3-methoxy-4-[(3-{4-[(1-methylpiperidin-4-yl)amino]-1-(2,2,2-trifluoroethyl)-1H- |
| | indol-2-yl}prop-2-yn-1-yl)amino]phenyl}methanesulfonamide |
| 374. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]benzonitrile |
| 375. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]benzoic acid |
| 376. | 2-{3-[(2,4-dimethoxyphenyl)amino]prop-1-yn-1-yl}-N-(1-methylpiperidin-4-yl)-1-(2,2,2- |
| | trifluoroethyl)-1H-indol-4-amine |
| 377. | 2-{3-[(2-methoxypyridin-3-yl)amino]prop-1-yn-1-yl}-N-(1-methylpiperidin-4-yl)-1- |
| | (2,2,2-trifluoroethyl)-1H-indol-4-amine |
| 378. | 2-{3-[(5-fluoro-2-methoxyphenyl)-amino]prop-1-yn-1-yl}-N-(1-methylpiperidin-4-yl)-1- |
| | (2,2,2-trifluoroethyl)-1H-indol-4-amine |
| 379. | 2-{3-[(2-ethoxy-4-methane-sulfonylphenyl)amino]prop-1-yn-1-yl}-N-(1-methylpiperidin- |
| | 4-yl)-1-(2,2,2-trifluoroethyl)-1H-indol-4-amine |
| 380. | 2-{3-[(3-fluoro-2-methoxyphenyl)-amino]prop-1-yn-1-yl}-N-(1-methylpiperidin-4-yl)-1- |
| | (2,2,2-trifluoroethyl)-1H-indol-4-amine |
| 381. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]benzene-1-sulfonamide |
| 382. | 2-{3-[(4-fluoro-2-methoxyphenyl)-amino]prop-1-yn-1-yl}-N-(1-methylpiperidin-4-yl)-1- |
| | (2,2,2-trifluoroethyl)-1H-indol-4-amine |
| 383. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-benzamide |
| 384. | 2-{3-[(2-fluoro-6-methoxyphenyl)-amino]prop-1-yn-1-yl}-N-(1-methylpiperidin-4-yl)-1- |
| | (2,2,2-trifluoroethyl)-1H-indol-4-amine |
| 385. | 2-{3-[(4-tert-butyl-2-methoxyphenyl)-amino]prop-1-yn-1-yl}-N-(1-methylpiperidin-4- |
| | yl)-1-(2,2,2-trifluoroethyl)-1H-indol-4-amine |
| 386. | 4-methoxy-3-[(3-{4-[(1-methylpiperidin-4-yl)amino]-1-(2,2,2-trifluoroethyl)-1H-indol-2- |
| | yl}prop-2-yn-1-yl)amino]-benzonitrile |
| 387. | 2-{3-[(5-tert-butyl-2-methoxyphenyl)-amino]prop-1-yn-1-yl}-N-(1-methylpiperidin-4- |
| | yl)-1-(2,2,2-trifluoroethyl)-1H-indol-4-amine |
| 388. | N-(1-methylpiperidin-4-yl)-2-[3-(phenylamino)prop-1-yn-1-yl]-1-(2,2,2-trifluoroethyl)- |
| | 1H-indol-4-amine |
| 389. | 5-methanesulfonyl-2-[(3-{4-[(1-methylpiperidin-4-yl)amino]-1-(2,2,2-trifluoroethyl)-1H- |
| | indol-2-yl}prop-2-yn-1-yl)amino]benzonitrile |
| 390. | 2-(3-{[2-(2-fluoroethoxy)-4-methanesulfonylphenyl]amino}prop-1-yn-1-yl)-N-(1- |
| | methylpiperidin-4-yl)-1-(2,2,2-trifluoroethyl)-1H-indol-4-amine |
| 391. | 2-{3-[(3-methoxypyridin-4-yl)amino]prop-1-yn-1-yl}-N-(1-methylpiperidin-4-yl)-1- |
| | (2,2,2-trifluoroethyl)-1H-indol-4-amine |
| 392. | 2-{3-[(2-chloro-4-methanesulfonyl-phenyl)amino]prop-1-yn-1-yl}-N-(1-methylpiperidin- |
| | 4-yl)-1-(2,2,2-trifluoroethyl)-1H-indol-4-amine |
| 393. | 2-{3-[(4-methoxyphenyl)amino]prop-1-yn-1-yl}-N-(1-methylpiperidin-4-yl)-1-(2,2,2- |
| | trifluoroethyl)-1H-indol-4-amine |
| 394. | 2-(3-{[4-methanesulfonyl-2-(2-methoxyethoxy)phenyl]amino}prop-1-yn-1-yl)-N-(1- |
| | methylpiperidin-4-yl)-1-(2,2,2-trifluoroethyl)-1H-indol-4-amine |
| 395. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]pyridine-2-carbonitrile |
| 396. | 4-{[2-(3-{[6-(morpholine-4-carbonyl)pyridin-3-yl]amino}prop-1-yn-1-yl)-1-(2,2,2- |
| | trifluoroethyl)-1H-indol-4-yl]amino}-1λ⁶-thiane-1,1-dione |
| 397. | 4-{[2-(3-{[6-(4-methylpiperazine-1-carbonyl)pyridin-3-yl]amino}prop-1-yn-1-yl)-1- |
| | (2,2,2-trifluoroethyl)-1H-indol-4-yl]amino}-1λ⁶-thiane-1,1-dione |
| 398. | 4-[(2-{3-[(quinolin-3-yl)amino]prop-1-yn-1-yl}-1-(2,2,2-trifluoroethyl)-1H-indol-4- |
| | yl)amino]-1λ⁶-thiane-1,1-dione |
| 399. | 4-[(2-{3-[(quinoxalin-6-yl)amino]prop-1-yn-1-yl}-1-(2,2,2-trifluoroethyl)-1H-indol-4- |
| | yl)amino]-1λ⁶-thiane-1,1-dione |
| 400. | 4-[(2-{3-[(4-methanesulfonylphenyl)-amino]prop-1-yn-1-yl}-1-(2,2,2-trifluoroethyl)-1H- |
| | indol-4-yl)amino]-1λ⁶-thiane-1,1-dione |
| 401. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]pyridine-2-carboxamide |
| 402. | 4-[(2-{3-[(6-methoxypyridin-3-yl)amino]prop-1-yn-1-yl}-1-(2,2,2-trifluoroethyl)-1H- |
| | indol-4-yl)amino]-1λ⁶-thiane-1,1-dione |
| 403. | 4-{[2-(3-{[6-(4-hydroxypiperidine-1-carbonyl)pyridin-3-yl]amino}prop-1-yn-1-yl)-1- |
| | (2,2,2-trifluoroethyl)-1H-indol-4-yl]amino}-1λ⁶-thiane-1,1-dione |
| 404. | 4-[(2-{3-[(1-methyl-2-oxo-1,2-dihydropyridin-4-yl)amino]prop-1-yn-1-yl}-1-(2,2,2- |
| | trifluoroethyl)-1H-indol-4-yl)amino]-1λ⁶-thiane-1,1-dione |
| 405. | 4-[(2-{3-[(2-methoxypyridin-4-yl)amino]prop-1-yn-1-yl}-1-(2,2,2-trifluoroethyl)-1H- |
| | indol-4-yl)amino]-1λ⁶-thiane-1,1-dione |
| 406. | 2-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-fluorophenyl}-2-methylpropanenitrile |
| 407. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(1-methylpiperidin-4-yl)pyridine-2-carboxamide |
| 408. | 4-[(2-{3-[(2-fluoro-4-methanesulfonyl-phenyl)amino]prop-1-yn-1-yl}-1-(2,2,2- |
| | trifluoroethyl)-1H-indol-4-yl)amino]-1λ⁶-thiane-1,1-dione |
| 409. | 4-{[2-(3-{[4-(ethanesulfonyl)-phenyl]amino}prop-1-yn-1-yl)-1-(2,2,2-trifluoroethyl)-1H- |
| | indol-4-yl]amino}-1λ⁶-thiane-1,1-dione |
| 410. | 4-[(2-{3-[(2-tert-butylpyrimidin-5-yl)amino]prop-1-yn-1-yl}-1-(2,2,2-trifluoroethyl)-1H- |
| | indol-4-yl)amino]-1λ⁶-thiane-1,1-dione |
| 411. | 3-{4-[(1,1-dioxo-1λ⁶-thian-4-yl)amino]-1-(2,2,2-trifluoroethyl)-1H-indol-2-yl}-N-(4- |
| | methanesulfonyl-phenyl)-prop-2-ynamide |
| 412. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(oxan-4-yl)pyridine-2-carboxamide |
| 413. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(pyridin-3-yl)pyridine-2-carboxamide |
| 414. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-methylpyridine-2-carboxamide |
| 415. | 4-[(2-{3-[(3-chloro-4-methanesulfonylphenyl)amino]prop-1-yn-1-yl}-1-(2,2,2- |
| | trifluoroethyl)-1H-indol-4-yl)amino]-1λ⁶-thiane-1,1-dione |
| 416. | N-(2,3-dihydroxypropyl)-5-[(3-{4-[(1,1-dioxo-1λ⁶-thian-4-yl)amino]-1-(2,2,2- |
| | trifluoroethyl)-1H-indol-2-yl}prop-2-yn-1-yl)amino]pyridine-2-carboxamide |
| 417. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-hydroxypyridine-2-carboxamide |
| 418. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(2-hydroxyethyl)pyridine-2-carboxamide |
| 419. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-hydroxy-N-methylpyridine-2-carboxamide |
| 420. | 4-amino-N-(3-{4-[(1,1-dioxo-1λ⁶-thian-4-yl)amino]-1-(2,2,2-trifluoroethyl)-1H-indol-2- |
| | yl}prop-2-yn-1-yl)benzene-1-sulfonamide |
| 421. | 4-({2-[3-({pyrido[2,3-b]pyrazin-7-yl}amino)prop-1-yn-1-yl]-1-(2,2,2-trifluoroethyl)-1H- |
| | indol-4-yl}amino)-1λ⁶-thiane-1,1-dione |
| 422. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-benzamide |
| 423. | 4-{[2-(3-{[2-(methylsulfanyl)pyrimidin-5-yl]amino}prop-1-yn-1-yl)-1-(2,2,2- |
| | trifluoroethyl)-1H-indol-4-yl]amino}-1λ⁶-thiane-1,1-dione |
| 424. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]benzene-1-sulfonamide |
| 425. | 4-{[2-(3-{[4-(2-methylpropane-2-sulfonyl)phenyl]amino}prop-1-yn-1-yl)-1-(2,2,2- |
| | trifluoroethyl)-1H-indol-4-yl]amino}-1λ⁶-thiane-1,1-dione |
| 426. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N,N-dimethylbenzene-1-sulfonamide |
| 427. | 4-{[1-(2,2,2-trifluoroethyl)-2-[2-(trimethylsilyl)ethynyl]-1H-indol-4-yl]amino}-1λ⁶- |
| | thiane-1,1-dione |
| 428. | 4-[(2-{3-[(5-methanesulfonylpyridin-2-yl)amino]prop-1-yn-1-yl}-1-(2,2,2-trifluoroethyl)- |
| | 1H-indol-4-yl)amino]-1λ⁶-thiane-1,1-dione |
| 429. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]-1λ⁶-thiane-1,1-dione |
| 430. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-methylbenzene-1-sulfonamide |
| 431. | 4-{[2-ethynyl-1-(2,2,2-trifluoroethyl)-1H-indol-4-yl]amino}-1λ⁶-thiane-1,1-dione |
| 432. | N-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}methanesulfonamide |
| 433. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzoic acid |
| 434. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzonitrile |
| 435. | 4-[(2-{3-[(5-fluoro-2-methoxyphenyl)amino]prop-1-yn-1-yl}-1-(2,2,2-trifluoroethyl)-1H- |
| | indol-4-yl)amino]-1λ⁶-thiane-1,1-dione |
| 436. | 4-[(2-{3-[(2-methoxy-6-methylpyridin-3-yl)amino]prop-1-yn-1-yl}-1-(2,2,2- |
| | trifluoroethyl)-1H-indol-4-yl)amino]-1λ⁶-thiane-1,1-dione |
| 437. | 4-[(2-{3-[(2-hydroxy-6-methylpyridin-3-yl)amino]prop-1-yn-1-yl}-1-(2,2,2- |
| | trifluoroethyl)-1H-indol-4-yl)amino]-1λ⁶-thiane-1,1-dione |
| 438. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzamide |
| 439. | 4-[(2-{3-[(2-methoxypyridin-3-yl)amino]prop-1-yn-1-yl}-1-(2,2,2-trifluoroethyl)-1H- |
| | indol-4-yl)amino]-1λ⁶-thiane-1,1-dione |
| 440. | 4-[(2-{3-[(4-fluoro-2-methoxyphenyl)amino]prop-1-yn-1-yl}-1-(2,2,2-trifluoroethyl)-1H- |
| | indol-4-yl)amino]-1λ⁶-thiane-1,1-dione |
| 441. | 4-[(2-{3-[(5-tert-butyl-2-methoxyphenyl)amino]prop-1-yn-1-yl}-1-(2,2,2-trifluoroethyl)- |
| | 1H-indol-4-yl)amino]-1λ⁶-thiane-1,1-dione |
| 442. | 4-[(2-{3-[(2-ethoxy-4-methanesulfonylphenyl)amino]prop-1-yn-1-yl}-1-(2,2,2- |
| | trifluoroethyl)-1H-indol-4-yl)amino]-1λ⁶-thiane-1,1-dione |
| 443. | 4-[(2-{3-[(3-fluoro-2-methoxyphenyl)-amino]prop-1-yn-1-yl}-1-(2,2,2-trifluoroethyl)- |
| | 1H-indol-4-yl)amino]-1λ⁶-thiane-1,1-dione |
| 444. | 4-({2-[3-(methylamino)prop-1-yn-1-yl]-1-(2,2,2-trifluoroethyl)-1H-indol-4-yl}amino)- |
| | 1λ⁶-thiane-1,1-dione |
| 445. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzene-1-sulfonamide |
| 446. | 4-[(2-{3-[(2-fluoro-6-methoxyphenyl)-amino]prop-1-yn-1-yl}-1-(2,2,2-trifluoroethyl)- |
| | 1H-indol-4-yl)amino]-1λ⁶-thiane-1,1-dione |
| 447. | 3-[(3-{4-[(1,1-dioxo-1λ⁶-thian-4-yl)amino]-1-(2,2,2-trifluoroethyl)-1H-indol-2-yl}prop-2- |
| | yn-1-yl)amino]-4-methoxybenzonitrile |
| 448. | 4-[(2-{3-[(4-tert-butyl-2-methoxy-phenyl)amino]prop-1-yn-1-yl}-1-(2,2,2-trifluoroethyl)- |
| | 1H-indol-4-yl)amino]-1λ⁶-thiane-1,1-dione |
| 449. | 4-({2-[3-(phenylamino)prop-1-yn-1-yl]-1-(2,2,2-trifluoroethyl)-1H-indol-4-yl}amino)- |
| | 1λ⁶-thiane-1,1-dione |
| 450. | 4-{[2-(3-{[2-(2-fluoroethoxy)-4-methanesulfonylphenyl]amino}prop-1-yn-1-yl)-1-(2,2,2- |
| | trifluoroethyl)-1H-indol-4-yl]amino}-1λ⁶-thiane-1,1-dione |
| 451. | 2-[(3-{4-[(1,1-dioxo-1λ⁶-thian-4-yl)amino]-1-(2,2,2-trifluoroethyl)-1H-indol-2-yl}prop-2- |
| | yn-1-yl)amino]-5-methanesulfonylbenzonitrile |
| 452. | 4-[(2-{3-[(2-chloro-4-methanesulfonyl-phenyl)amino]prop-1-yn-1-yl}-1-(2,2,2- |
| | trifluoroethyl)-1H-indol-4-yl)amino]-1λ⁶-thiane-1,1-dione |
| 453. | 4-[(2-{3-[(4-methoxyphenyl)amino]prop-1-yn-1-yl}-1-(2,2,2-trifluoroethyl)-1H-indol-4- |
| | yl)amino]-1λ⁶-thiane-1,1-dione |
| 454. | 4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}-1λ⁶-thiane-1,1-dione |
| 455. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-N,N-dimethylacetamide |
| 456. | 2-methyl-2-(5-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}pyridin-2-yl)propanenitrile |
| 457. | 2-(5-((3-(4-(((1S,4S)-4-(dimethylamino)-cyclohexyl)amino)-1-(2,2,2-trifluoroethyl)-1H- |
| | indol-2-yl)prop-2-yn-1-yl)amino)pyridin-2-yl)-2-methylpropanenitrile |
| 458. | N-[1-(2-methanesulfonylethyl)piperidin-4-yl]-2-{3-[(4-methanesulfonylphenyl)- |
| | amino]prop-1-yn-1-yl}-1-(2,2,2-trifluoroethyl)-1H-indol-4-amine |
| 459. | 5-({3-[4-({1-[(dimethylcarbamoyl)methyl]piperidin-4-yl}amino)-1-(2,2,2-trifluoroethyl)- |
| | 1H-indol-2-yl]prop-2-yn-1-yl}amino)pyridine-2-carboxamide |
| 460. | 5-{[3-(4-{[1-(2-methanesulfonylethyl)piperidin-4-yl]amino}-1-(2,2,2-trifluoroethyl)-1H- |
| | indol-2-yl)prop-2-yn-1-yl]amino}pyridine-2-carboxamide |
| 461. | 5-[(3-{4-[(piperidin-4-yl)amino]-1-(2,2,2-trifluoroethyl)-1H-indol-2-yl}prop-2-yn-1- |
| | yl)amino]pyridine-2-carboxamide |
| 462. | 5-{[3-(4-{[1-(carbamoylmethyl)piperidin-4-yl]amino}-1-(2,2,2-trifluoroethyl)-1H-indol- |
| | 2-yl)prop-2-yn-1-yl]amino}pyridine-2-carboxamide |
| 463. | 5-{[3-(4-{[1-(2-hydroxyethyl)piperidin-4-yl]amino}-1-(2,2,2-trifluoroethyl)-1H-indol-2- |
| | yl)prop-2-yn-1-yl]amino}pyridine-2-carboxamide |
| 464. | 5-{[3-(4-{[1-(oxan-4-yl)piperidin-4-yl]amino}-1-(2,2,2-trifluoroethyl)-1H-indol-2- |
| | yl)prop-2-yn-1-yl]amino}pyridine-2-carboxamide |
| 465. | 5-{[3-(4-{[(1R,4R)-4-(dimethylamino)-cyclohexyl]amino}-1-(2,2,2-trifluoroethyl)-1H- |
| | indol-2-yl)prop-2-yn-1-yl]amino}pyridine-2-carboxamide |
| 466. | 4-{[3-(4-{[1-(2-methanesulfonyl-ethyl)piperidin-4-yl]amino}-1-(2,2,2-trifluoroethyl)-1H- |
| | indol-2-yl)prop-2-yn-1-yl]amino}benzene-1-sulfonamide |
| 467. | 4-{[3-(4-{[1-(2-methoxyethyl)piperidin-4-yl]amino}-1-(2,2,2-trifluoroethyl)-1H-indol-2- |
| | yl)prop-2-yn-1-yl]amino}benzene-1-sulfonamide |
| 468. | 5-{[3-(4-{[(1S,4S)-4-(dimethylamino)-cyclohexyl]amino}-1-(2,2,2-trifluoroethyl)-1H- |
| | indol-2-yl)prop-2-yn-1-yl]amino}pyridine-2-carboxamide |
| 469. | N,N-dimethyl-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzene-1-sulfonamide |
| 470. | 4-{[3-(4-{[1-(oxan-4-yl)piperidin-4-yl]amino}-1-(2,2,2-trifluoroethyl)-1H-indol-2- |
| | yl)prop-2-yn-1-yl]amino}benzene-1-sulfonamide |
| 471. | 2-{4-[(2-{3-[(4-sulfamoylphenyl)-amino]prop-1-yn-1-yl}-1-(2,2,2-trifluoroethyl)-1H- |
| | indol-4-yl)amino]-piperidin-1-yl}acetamide |
| 472. | 4-{[3-(4-{[1-(2-methoxyethyl)piperidin-4-yl]amino}-1-(2,2,2-trifluoroethyl)-1H-indol-2- |
| | yl)prop-2-yn-1-yl]amino}-N,N-dimethylbenzene-1-sulfonamide |
| 473. | 4-[(3-{4-[(oxan-4-yl)amino]-1-(2,2,2-trifluoroethyl)-1H-indol-2-yl}prop-2-yn-1- |
| | yl)amino]benzene-1-sulfonamide |
| 474. | 4-{[3-(4-{[1-(2-hydroxypropyl)piperidin-4-yl]amino}-1-(2,2,2-trifluoroethyl)-1H-indol-2- |
| | yl)prop-2-yn-1-yl]amino}benzene-1-sulfonamide |
| 475. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}benzene-1-sulfonamide |
| 476. | 4-({3-[4-({1-[2-(morpholin-4-yl)-2-oxoethyl]piperidin-4-yl}amino)-1-(2,2,2- |
| | trifluoroethyl)-1H-indol-2-yl]prop-2-yn-1-yl}amino)benzene-1-sulfonamide |
| 477. | methyl 2-{4-[(2-{3-[(4-sulfamoylphenyl)-amino]prop-1-yn-1-yl}-1-(2,2,2-trifluoro- |
| | ethyl)-1H-indol-4-yl)amino]piperidin-1-yl}acetate |
| 478. | 4-{[3-(4-{[1-(2-hydroxyethyl)piperidin-4-yl]amino}-1-(2,2,2-trifluoroethyl)-1H-indol-2- |
| | yl)prop-2-yn-1-yl]amino}benzene-1-sulfonamide |
| 479. | 4-({3-[4-({1-[2-(2-hydroxyethoxy)ethyl]piperidin-4-yl}amino)-1-(2,2,2-trifluoroethyl)- |
| | 1H-indol-2-yl]prop-2-yn-1-yl}amino)benzene-1-sulfonamide |
| 480. | N,N-dimethyl-2-{4-[(2-{3-[(4-sulfamoylphenyl)amino]prop-1-yn-1-yl}-1-(2,2,2- |
| | trifluoroethyl)-1H-indol-4-yl)amino]piperidin-1-yl}acetamide |
| 481. | 4-({3-[4-({1-[2-(4-hydroxypiperidin-1-yl)-2-oxoethyl]piperidin-4-yl}amino)-1-(2,2,2- |
| | trifluoroethyl)-1H-indol-2-yl]prop-2-yn-1-yl}amino)benzene-1-sulfonamide |
| 482. | 2-{4-[(2-{3-[(4-sulfamoylphenyl)amino]prop-1-yn-1-yl}-1-(2,2,2-trifluoroethyl)-1H- |
| | indol-4-yl)amino]piperidin-1-yl}acetic acid |
| 483. | 4-({3-[4-({1-[2-(4-methylpiperazin-1-yl)-2-oxoethyl]piperidin-4-yl}amino)-1-(2,2,2- |
| | trifluoroethyl)-1H-indol-2-yl]prop-2-yn-1-yl}amino)benzene-1-sulfonamide |
| 484. | N-methyl-2-{4-[(2-{3-[(4-sulfamoylphenyl)amino]prop-1-yn-1-yl}-1-(2,2,2- |
| | trifluoroethyl)-1H-indol-4-yl)amino]piperidin-1-yl}acetamide |
| 485. | N-methyl-4-[(3-{4-[(oxan-4-yl)amino]-1-(2,2,2-trifluoroethyl)-1H-indol-2-yl}prop-2-yn- |
| | 1-yl)amino]benzene-1-sulfonamide |
| 486. | 4-{[3-(4-{[1-(2-methoxyethyl)piperidin-4-yl]amino}-1-(2,2,2-trifluoroethyl)-1H-indol-2- |
| | yl)prop-2-yn-1-yl]amino}-N-methylbenzene-1-sulfonamide |
| 487. | N-methyl-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzene-1-sulfonamide |
| 488. | 2-(dimethylamino)ethyl 2-{4-[(2-{3-[(4-methanesulfonylphenyl)-amino]prop-1-yn-1-yl}- |
| | 1-(2,2,2-trifluoroethyl)-1H-indol-4-yl)amino]piperidin-1-yl}acetate |
| 489. | 2-methoxy-4-[(3-{4-[(oxan-4-yl)amino]-1-(2,2,2-trifluoroethyl)-1H-indol-2-yl}prop-2- |
| | yn-1-yl)amino]benzene-1-sulfonamide |
| 490. | 2-chloro-4-[(3-{4-[(oxan-4-yl)amino]-1-(2,2,2-trifluoroethyl)-1H-indol-2-yl}prop-2-yn-1- |
| | yl)amino]benzene-1-sulfonamide |
| 491. | 3-methoxy-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzene-1-sulfonamide |
| 492. | 4-{[3-(4-{[1-(2,3-dihydroxypropyl)-piperidin-4-yl]amino}-1-(2,2,2-trifluoro-ethyl)-1H- |
| | indol-2-yl)prop-2-yn-1-yl]amino}-3-methoxybenzene-1-sulfonamide |
| 493. | 3-methoxy-4-[(3-{4-[(piperidin-4-yl)amino]-1-(2,2,2-trifluoroethyl)-1H-indol-2-yl}prop- |
| | 2-yn-1-yl)amino]benzene-1-sulfonamide |
| 494. | 3-methoxy-4-[(3-{4-[(oxan-4-yl)amino]-1-(2,2,2-trifluoroethyl)-1H-indol-2-yl}prop-2- |
| | yn-1-yl)amino]benzene-1-sulfonamide |
| 495. | 3-methoxy-4-[(3-{4-[(oxan-4-yl)amino]-1-(2,2,2-trifluoroethyl)-1H-indol-2-yl}prop-2- |
| | yn-1-yl)amino]benzamide |
| 496. | 3-methoxy-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzamide |
| 497. | 4-{[3-(4-{[1-(2,3-dihydroxypropyl)-piperidin-4-yl]amino}-1-(2,2,2-trifluoroethyl)-1H- |
| | indol-2-yl)prop-2-yn-1-yl]amino}-3-methoxybenzamide |
| 498. | 2-[5-({3-[1-(cyanomethyl)-4-[(1-methylpiperidin-4-yl)amino]-1H-indol-2-yl]prop-2-yn-1- |
| | yl}amino)pyridin-2-yl]-2-methylpropanenitrile |
| 499. | 2-[5-({3-[1-(3-methoxypropyl)-4-[(1-methylpiperidin-4-yl)amino]-1H-indol-2-yl]prop-2- |
| | yn-1-yl}amino)pyridin-2-yl]-2-methylpropanenitrile |
| 500. | 2-[5-({3-[1-(2-chloroethyl)-4-[(1-methylpiperidin-4-yl)amino]-1H-indol-2-yl]prop-2-yn- |
| | 1-yl}amino)pyridin-2-yl]-2-methylpropanenitrile |
| 501. | 2-methyl-2-{5-[(3-{4-[(1-methylpiperidin-4-yl)amino]-1-(propan-2-yl)-1H-indol-2- |
| | yl}prop-2-yn-1-yl)amino]pyridin-2-yl}propanenitrile |
| 502. | 2-{5-[(3-{1-cyclopentyl-4-[(1-methylpiperidin-4-yl)amino]-1H-indol-2-yl}prop-2-yn-1- |
| | yl)amino]pyridin-2-yl}-2-methylpropanenitrile |
| 503. | 2-methyl-2-{5-[(3-{4-[(1-methylpiperidin-4-yl)amino]-1-(3,3,3-trifluoropropyl)-1H- |
| | indol-2-yl}prop-2-yn-1-yl)amino]pyridin-2-yl}propanenitrile |
| 504. | 1-(2-chloroethyl)-N-(1-methylpiperidin-4-yl)-2-{3-[(6-methylpyridin-3-yl)amino]-prop-1- |
| | yn-1-yl}-1H-indol-4-amine |
| 505. | 1-(2-chloroethyl)-N-(1-methylpiperidin-4-yl)-2-{3-[(6-methylpyridin-3-yl)amino]-prop-1- |
| | yn-1-yl}-1H-indol-4-amine |
| 506. | 1-(2-chloroethyl)-2-{3-[(4-chlorophenyl)-amino]prop-1-yn-1-yl}-N-(1-methylpiperidin-4- |
| | yl)-1H-indol-4-amine |
| 507. | 2-[5-({3-[1-(1-cyanoethyl)-4-[(1-methylpiperidin-4-yl)amino]-1H-indol-2-yl]prop-2-yn- |
| | 1-yl}amino)pyridin-2-yl]-2-methylpropanenitrile |
| 508. | 2-[5-({3-[1-(cyanomethyl)-4-[(1,1-dioxo-1λ⁶-thian-4-yl)amino]-1H-indol-2-yl]prop-2-yn- |
| | 1-yl}amino)pyridin-2-yl]-2-methylpropanenitrile |
| 509. | 4-[(2-{3-[(4-methanesulfonylphenyl)amino]prop-1-yn-1-yl}-1-(oxiran-2-ylmethyl)-1H- |
| | indol-4-yl)amino]-1λ⁶-thiane-1,1-dione |
| 510. | 2-{3-[(4-methanesulfonylphenyl)-amino]prop-1-yn-1-yl}-N-[1-(oxan-4-yl)piperidin-4- |
| | yl]-1-(oxiran-2-ylmethyl)-1H-indol-4-amine |
| 511. | 4-[(2-{3-[(4-methanesulfonyl-2-methoxyphenyl)amino]prop-1-yn-1-yl}-1-(oxiran-2- |
| | ylmethyl)-1H-indol-4-yl)amino]-1λ⁶-thiane-1,1-dione |
| 512. | 2-{3-[(4-methanesulfonyl-2-methoxy-phenyl)amino]prop-1-yn-1-yl}-N-[1-(oxan-4- |
| | yl)piperidin-4-yl]-1-(oxiran-2-ylmethyl)-1H-indol-4-amine |
| 513. | 1-[6-(1-cyano-1-methylethyl)pyridin-3-yl]-3-(3-{4-[(1-methylpiperidin-4-yl)amino]-1- |
| | (2,2,2-trifluoroethyl)-1H-indol-2-yl}prop-2-yn-1-yl)urea |
| 514. | 1-(6-methanesulfonylpyridin-3-yl)-3-(3-{4-[(1-methylpiperidin-4-yl)amino]-1-(2,2,2- |
| | trifluoroethyl)-1H-indol-2-yl}prop-2-yn-1-yl)urea |
| 515. | 1-[6-(1-cyano-1-methylethyl)pyridin-3-yl]-3-(3-{4-[(1,1-dioxo-1λ⁶-thian-4-yl)amino]-1- |
| | (2,2,2-trifluoroethyl)-1H-indol-2-yl}prop-2-yn-1-yl)urea |
| 516. | 3-(3-{4-[(1,1-dioxo-1λ⁶-thian-4-yl)amino]-1-(2,2,2-trifluoroethyl)-1H-indol-2-yl}prop-2- |
| | yn-1-yl)-1-(6-methanesulfonylpyridin-3-yl)urea |
| 517. | 1-(6-cyanopyridin-3-yl)-3-(3-{4-[(1-methylpiperidin-4-yl)amino]-1-(2,2,2-trifluoroethyl)- |
| | 1H-indol-2-yl}prop-2-yn-1-yl)urea |
| 518. | 1-(6-cyanopyridin-3-yl)-3-(3-{4-[(1,1-dioxo-1λ⁶-thian-4-yl)amino]-1-(2,2,2- |
| | trifluoroethyl)-1H-indol-2-yl}prop-2-yn-1-yl)urea |
| 519. | 3-(3-{4-[(1,1-dioxo-1λ⁶-thian-4-yl)amino]-1-(2,2,2-trifluoroethyl)-1H-indol-2-yl}prop-2- |
| | yn-1-yl)-1-(quinoxalin-6-yl)urea |
| 520. | N-(3-{4-[(1,1-dioxo-1λ⁶-thian-4-yl)amino]-1-(2,2,2-trifluoroethyl)-1H-indol-2-yl}prop-2- |
| | yn-1-yl)-4-methylpiperazine-1-carboxamide |
| 521. | N-(3-{4-[(1,1-dioxo-1λ⁶-thian-4-yl)amino]-1-(2,2,2-trifluoroethyl)-1H-indol-2-yl}prop-2- |
| | yn-1-yl)morpholine-4-carboxamide |
| 522. | 4-[(2-{3-[(6-methanesulfonylpyridin-3-yl)amino]prop-1-yn-1-yl}-1-(2,2,2-trifluoroethyl)- |
| | 1H-indol-4-yl)amino]-1λ⁶-thiane-1,1-dione |
| 523. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-N,N-dimethylacetamide |
| 524. | N-(1-ethylpiperidin-4-yl)-2-{3-[(6-methanesulfonylpyridin-3-yl)amino]prop-1-yn-1-yl}- |
| | 1-(2,2,2-trifluoroethyl)-1H-indol-4-amine |
| 525. | N-[1-(2-methanesulfonylethyl)piperidin-4-yl]-2-{3-[(6-methanesulfonylpyridin-3- |
| | yl)amino]prop-1-yn-1-yl}-1-(2,2,2-trifluoroethyl)-1H-indol-4-amine |
| 526. | 2-{3-[(6-methanesulfonylpyridin-3-yl)amino]prop-1-yn-1-yl}-N-[1-(1-methylpiperidin-4- |
| | yl)piperidin-4-yl]-1-(2,2,2-trifluoroethyl)-1H-indol-4-amine |
| 527. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}ethan-1-ol |
| 528. | 4-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-1λ⁶-thiane-1,1-dione |
| 529. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-1-(4-methylpiperazin-1-yl)ethan-1- |
| | one |
| 530. | 2-{3-[(4-methanesulfonylphenyl)amino]prop-1-yn-1-yl}-N-(oxan-4-yl)-1-(2,2,2- |
| | trifluoroethyl)-1H-indol-4-amine |
| 531. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}ethan-1-ol |
| 532. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-1-(4-methylpiperazin-1-yl)ethan-1-one |
| 533. | 2-{3-[(4-methanesulfonylphenyl)amino]prop-1-yn-1-yl}-N-[1-(1-methylpiperidin-4- |
| | yl)piperidin-4-yl]-1-(2,2,2-trifluoroethyl)-1H-indol-4-amine |
| 534. | N-(2,3-dihydroxypropyl)-2-{4-[(2-{3-[(4-methanesulfonylphenyl)-amino]prop-1-yn-1- |
| | yl}-1-(2,2,2-trifluoroethyl)-1H-indol-4-yl)amino]piperidin-1-yl}-N-methylacetamide |
| 535. | 4-N-(2-{3-[(4-methanesulfonylphenyl)-amino]prop-1-yn-1-yl}-1-(2,2,2-trifluoroethyl)- |
| | 1H-indol-4-yl)-1-N,1-N-dimethylcyclohexane-1,4-diamine |
| 536. | (1S,4S)-4-N-(2-{3-[(4-methanesulfonyl-phenyl)amino]prop-1-yn-1-yl}-1-(2,2,2- |
| | trifluoroethyl)-1H-indol-4-yl)-1-N,1-N-dimethylcyclohexane-1,4-diamine |
| 537. | 2-{3-[(4-methanesulfonylphenyl)-amino]prop-1-yn-1-yl}-N-[1-(oxan-4-yl)piperidin-4- |
| | yl]-1-(2,2,2-trifluoroethyl)-1H-indol-4-amine |
| 538. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-1-(morpholin-4-yl)ethan-1-one |
| 539. | 1-(4-hydroxypiperidin-1-yl)-2-{4-[(2-{3-[(4-methanesulfonylphenyl)-amino]prop-1-yn-1- |
| | yl}-1-(2,2,2-trifluoroethyl)-1H-indol-4-yl)amino]piperidin-1-yl}ethan-1-one |
| 540. | 2-{3-[(4-methanesulfonylphenyl)-amino]prop-1-yn-1-yl}-N-(piperidin-4-yl)-1-(2,2,2- |
| | trifluoroethyl)-1H-indol-4-amine |
| 541. | N-{1-[1-(2-methanesulfonylethyl)piperidin-4-yl]piperidin-4-yl}-2-{3-[(4- |
| | methanesulfonylphenyl)amino]prop-1-yn-1-yl}-1-(2,2,2-trifluoroethyl)-1H-indol-4-amine |
| 542. | 2-{3-[(4-methanesulfonylphenyl)-amino]prop-1-yn-1-yl}-N-[1-(2- |
| | methoxyethyl)piperidin-4-yl]-1-(2,2,2-trifluoroethyl)-1H-indol-4-amine |
| 543. | 3-{4-[(2-{3-[(4-methane-sulfonylphenyl)-amino]prop-1-yn-1-yl}-1-(2,2,2-trifluoroethyl)- |
| | 1H-indol-4-yl)amino]piperidin-1-yl}propanenitrile |
| 544. | 2-{3-[(4-methanesulfonylphenyl)-amino]prop-1-yn-1-yl}-N-(1-methylpiperidin-4-yl)-1- |
| | (2,2,2-trifluoroethyl)-1H-indol-4-amine |
| 545. | 2-{4-[(2-{3-[(4-methane-sulfonylphenyl)-amino]prop-1-yn-1-yl}-1-(2,2,2-trifluoroethyl)- |
| | 1H-indol-4-yl)amino]piperidin-1-yl}acetamide |
| 546. | 4-{4-[(2-{3-[(4-methane-sulfonylphenyl)-amino]prop-1-yn-1-yl}-1-(2,2,2-trifluoroethyl)- |
| | 1H-indol-4-yl)amino]piperidin-1-yl}-1λ⁶-thiane-1,1-dione |
| 547. | 2-{4-[(2-{3-[(4-methane-sulfonylphenyl)amino]prop-1-yn-1-yl}-1-(2,2,2-trifluoroethyl)- |
| | 1H-indol-4-yl)amino]piperidin-1-yl}-N-methylacetamide |
| 548. | 2-(3-{[4-(ethanesulfonyl)phenyl]amino}prop-1-yn-1-yl)-N-(1-methylpiperidin-4-yl)-1- |
| | (2,2,2-trifluoroethyl)-1H-indol-4-amine |
| 549. | 2-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}ethan-1-ol |
| 550. | 2-{3-[(2-fluoro-4-methane-sulfonylphenyl)-amino]prop-1-yn-1-yl}-N-[1-(2- |
| | methanesulfonylethyl)-piperidin-4-yl]-1-(2,2,2-trifluoroethyl)-1H-indol-4-amine |
| 551. | 2-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}acetamide |
| 552. | 2-{3-[(2-fluoro-4-methane-sulfonylphenyl)-amino]prop-1-yn-1-yl}-N-(1-methylpiperidin- |
| | 4-yl)-1-(2,2,2-trifluoroethyl)-1H-indol-4-amine |
| 553. | 1-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}-2-methoxyethan-1-one |
| 554. | 2-{3-[(4-methanesulfonyl-phenyl)amino]prop-1-yn-1-yl}-N-(1-methylpyrrolidin-3-yl)-1- |
| | (2,2,2-trifluoroethyl)-1H-indol-4-amine |
| 555. | N-hydroxy-2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}acetamide |
| 556. | 3-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}propane-1,2-diol |
| 557. | 2-{3-[(2-fluoro-4-methanesulfonyl-phenyl)amino]prop-1-yn-1-yl}-N-[1-(1- |
| | methylpiperidin-4-yl)piperidin-4-yl]-1-(2,2,2-trifluoroethyl)-1H-indol-4-amine |
| 558. | 2-(3-{[4-(ethanesulfonyl)phenyl]amino}prop-1-yn-1-yl)-N-[1-(1-methylpiperidin-4- |
| | yl)piperidin-4-yl]-1-(2,2,2-trifluoroethyl)-1H-indol-4-amine |
| 559. | 2-{3-[(2-fluoro-4-methanesulfonyl-phenyl)amino]prop-1-yn-1-yl}-N-(oxan-4-yl)-1- |
| | (2,2,2-trifluoroethyl)-1H-indol-4-amine |
| 560. | 2-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)ethan-1-ol |
| 561. | 1-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}propan-2-ol |
| 562. | 2-(3-{[4-(ethanesulfonyl)-phenyl]amino}prop-1-yn-1-yl)-N-[1-(2- |
| | methanesulfonylethyl)piperidin-4-yl]-1-(2,2,2-trifluoroethyl)-1H-indol-4-amine |
| 563. | 4-{[1-(2,2,2-trifluoroethyl)-2-{3-[(4-trifluoromethanesulfonylphenyl)amino]prop-1-yn-1- |
| | yl}-1H-indol-4-yl]amino}-1λ⁶-thiane-1,1-dione |
| 564. | 2-(4-{[1-(2,2,2-trifluoroethyl)-2-{3-[(4-trifluoromethanesulfonylphenyl)amino]prop-1-yn- |
| | 1-yl}-1H-indol-4-yl]amino}piperidin-1-yl)ethan-1-ol |
| 565. | N-(1-methylpiperidin-4-yl)-1-(2,2,2-trifluoroethyl)-2-{3-[(4- |
| | trifluoromethanesulfonylphenyl)amino]prop-1-yn-1-yl}-1H-indol-4-amine |
| 566. | 2-{3-[(4-methanesulfonyl-phenyl)amino]prop-1-yn-1-yl}-N-[1-(1-methylpyrrolidin-3- |
| | yl)piperidin-4-yl]-1-(2,2,2-trifluoroethyl)-1H-indol-4-amine |
| 567. | 2-{3-[(4-methanesulfonyl-phenyl)amino]prop-1-yn-1-yl}-N-[1-(3- |
| | methanesulfonylpropyl)piperidin-4-yl]-1-(2,2,2-trifluoroethyl)-1H-indol-4-amine |
| 568. | 2-(3-{[4-(ethanesulfonyl)-phenyl]amino}prop-1-yn-1-yl)-N-[1-(2- |
| | methoxyethyl)piperidin-4-yl]-1-(2,2,2-trifluoroethyl)-1H-indol-4-amine |
| 569. | 4-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)-1λ⁶-thiane-1,1-dione |
| 570. | 2-(3-{[4-(ethanesulfonyl)-phenyl]amino}prop-1-yn-1-yl)-N-[1-(oxan-4-yl)piperidin-4-yl]- |
| | 1-(2,2,2-trifluoroethyl)-1H-indol-4-amine |
| 571. | N-[1-(2-methanesulfonylethyl)-piperidin-4-yl]-1-(2,2,2-trifluoroethyl)-2-{3-[(4- |
| | trifluoromethane-sulfonylphenyl)amino]prop-1-yn-1-yl}-1H-indol-4-amine |
| 572. | N-[1-(oxan-4-yl)piperidin-4-yl]-1-(2,2,2-trifluoroethyl)-2-{3-[(4- |
| | trifluoromethanesulfonylphenyl)amino]prop-1-yn-1-yl}-1H-indol-4-amine |
| 573. | N-[1-(1-methylpiperidin-4-yl)piperidin-4-yl]-1-(2,2,2-trifluoroethyl)-2-{3-[(4- |
| | trifluoromethanesulfonylphenyl)amino]prop-1-yn-1-yl}-1H-indol-4-amine |
| 574. | 2-{3-[(2-fluoro-4-methanesulfonyl-phenyl)amino]prop-1-yn-1-yl}-N-[1-(oxan-4- |
| | yl)piperidin-4-yl]-1-(2,2,2-trifluoroethyl)-1H-indol-4-amine |
| 575. | 2-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}acetonitrile |
| 576. | 2-{3-[(2-fluoro-4-methanesulfonylphenyl)amino]prop-1-yn-1-yl}-N-[1-(2- |
| | methoxyethyl)piperidin-4-yl]-1-(2,2,2-trifluoroethyl)-1H-indol-4-amine |
| 577. | 2-{3-[(3-chloro-4-methanesulfonylphenyl)amino]prop-1-yn-1-yl}-N-[1-(oxan-4- |
| | yl)piperidin-4-yl]-1-(2,2,2-trifluoroethyl)-1H-indol-4-amine |
| 578. | 2-{3-[(2,6-difluoro-4-methanesulfonyl-phenyl)amino]prop-1-yn-1-yl}-N-[1-(oxan-4- |
| | yl)piperidin-4-yl]-1-(2,2,2-trifluoroethyl)-1H-indol-4-amine |
| 579. | 2-{3-[(3-chloro-4-methanesulfonyl-phenyl)amino]prop-1-yn-1-yl}-N-[1-(2- |
| | methoxyethyl)piperidin-4-yl]-1-(2,2,2-trifluoroethyl)-1H-indol-4-amine |
| 580. | 2-{4-[(2-{3-[(3-chloro-4-methanesulfonylphenyl)amino]prop-1-yn-1-yl}-1-(2,2,2- |
| | trifluoroethyl)-1H-indol-4-yl)amino]piperidin-1-yl}ethan-1-ol |
| 581. | (2S)-3-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}propane-1,2-diol |
| 582. | N-(5-aminopentyl)-2-{4-[(2-{3-[(4-methanesulfonylphenyl)amino]prop-1-yn-1-yl}-1- |
| | (2,2,2-trifluoroethyl)-1H-indol-4-yl)amino]piperidin-1-yl}acetamide |
| 583. | 2-{3-[(2,6-difluoro-4-methanesulfonyl-phenyl)amino]prop-1-yn-1-yl}-N-[1-(2- |
| | methoxyethyl)piperidin-4-yl]-1-(2,2,2-trifluoroethyl)-1H-indol-4-amine |
| 584. | 2-(3-{[4-(ethanesulfonyl)phenyl]-amino}prop-1-yn-1-yl)-N-(oxan-4-yl)-1-(2,2,2- |
| | trifluoroethyl)-1H-indol-4-amine |
| 585. | 2-(4-{[2-(3-{[4-(ethanesulfonyl)phenyl]amino}prop-1-yn-1-yl)-1-(2,2,2-trifluoroethyl)- |
| | 1H-indol-4-yl]amino}piperidin-1-yl)acetonitrile |
| 586. | 2-(3-{[4-(2-methylpropane-2-sulfonyl)phenyl]amino}prop-1-yn-1-yl)-N-[1-(oxan-4- |
| | yl)piperidin-4-yl]-1-(2,2,2-trifluoroethyl)-1H-indol-4-amine |
| 587. | 2-(2-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}ethoxy)ethan-1-ol |
| 588. | 1-{4-[(2-{3-[(2-fluoro-4-methane-sulfonylphenyl)amino]prop-1-yn-1-yl}-1-(2,2,2- |
| | trifluoroethyl)-1H-indol-4-yl)amino]piperidin-1-yl}propan-2-ol |
| 589. | 3-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}propane-1,2-diol |
| 590. | (1S,4S)-4-N-(2-{3-[(2-fluoro-4-methanesulfonylphenyl)amino]prop-1-yn-1-yl}-1-(2,2,2- |
| | trifluoroethyl)-1H-indol-4-yl)-1-N,1-N-dimethylcyclohexane-1,4-diamine |
| 591. | 3-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)propane-1,2-diol |
| 592. | 2-(3-{[4-(ethanesulfonyl)-phenyl]amino}prop-1-yn-1-yl)-N-[1-(3- |
| | methanesulfonylpropyl)-piperidin-4-yl]-1-(2,2,2-trifluoroethyl)-1H-indol-4-amine |
| 593. | 1-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)propan-2-ol |
| 594. | 2-[2-(4-{[2-(3-{[4-(ethanesulfonyl)-phenyl]amino}prop-1-yn-1-yl)-1-(2,2,2- |
| | trifluoroethyl)-1H-indol-4-yl]amino}piperidin-1-yl)ethoxy]ethan-1-ol |
| 595. | (1R,4R)-4-N-(2-{3-[(2-fluoro-4-methanesulfonylphenyl)amino]prop-1-yn-1-yl}-1-(2,2,2- |
| | trifluoroethyl)-1H-indol-4-yl)-1-N,1-N-dimethyl-cyclohexane-1,4-diamine |
| 596. | 2-{3-[(2,6-difluoro-4-methanesulfonylphenyl)amino]prop-1-yn-1-yl}-N-[1-(2- |
| | methanesulfonylethyl)piperidin-4-yl]-1-(2,2,2-trifluoroethyl)-1H-indol-4-amine |
| 597. | 4-{4-[(2-{3-[(2,6-difluoro-4-methanesulfonylphenyl)amino]prop-1-yn-1-yl}-1-(2,2,2- |
| | trifluoroethyl)-1H-indol-4-yl)amino]piperidin-1-yl}-1λ⁶⁻thiane-1,1-dione |
| 598. | 2-{3-[(4-methanesulfonyl-3-methoxyphenyl)amino]prop-1-yn-1-yl}-N-[1-(oxan-4- |
| | yl)piperidin-4-yl]-1-(2,2,2-trifluoroethyl)-1H-indol-4-amine |
| 599. | 2-{3-[(4-methanesulfonyl-3-methoxyphenyl)amino]prop-1-yn-1-yl}-N-[1-(2- |
| | methoxyethyl)piperidin-4-yl]-1-(2,2,2-trifluoroethyl)-1H-indol-4-amine |
| 600. | 4-N-(2-{3-[(2-fluoro-4-methanesulfonylphenyl)amino]prop-1-yn-1-yl}-1-(2,2,2- |
| | trifluoroethyl)-1H-indol-4-yl)-1-N,1-N-dimethylcyclohexane-1,4-diamine |
| 601. | 2-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}acetic acid |
| 602. | 2-hydroxyethyl 2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}acetate |
| 603. | 2-{3-[(4-methanesulfonylphenyl)-amino]prop-1-yn-1-yl}-N-(2-methylpiperidin-4-yl)-1- |
| | (2,2,2-trifluoroethyl)-1H-indol-4-amine |
| 604. | 2-{4-[(2-{3-[(4-methanesulfonyl-3-methoxyphenyl)amino]prop-1-yn-1-yl}-1-(2,2,2- |
| | trifluoroethyl)-1H-indol-4-yl)amino]piperidin-1-yl}ethan-1-ol |
| 605. | (2S)-2-(2-{4-[(2-{3-[(4-methane-sulfonylphenyl)amino]prop-1-yn-1-yl}-1-(2,2,2- |
| | trifluoroethyl)-1H-indol-4-yl)amino]piperidin-1-yl}acetamido)pentanedioic acid |
| 606. | 1,5-dimethyl (2S)-2-(2-{4-[(2-{3-[(4-methanesulfonylphenyl)amino]prop-1-yn-1-yl}-1- |
| | (2,2,2-trifluoroethyl)-1H-indol-4-yl)amino]piperidin-1-yl}acetamido)pentanedioate |
| 607. | N-(4-carbamimidamidobutyl)-2-{4-[(2-{3-[(4-methanesulfonylphenyl)-amino]prop-1-yn- |
| | 1-yl}-1-(2,2,2-trifluoroethyl)-1H-indol-4-yl)amino]piperidin-1-yl}acetamide |
| 608. | 2-{3-[(4-methanesulfonyl-2-methoxyphenyl)amino]prop-1-yn-1-yl}-N-(oxan-4-yl)-1- |
| | (2,2,2-trifluoroethyl)-1H-indol-4-amine |
| 609. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethan-1-ol |
| 610. | 2-{3-[(5-methanesulfonylpyridin-2-yl)amino]prop-1-yn-1-yl}-N-(oxan-4-yl)-1-(2,2,2- |
| | trifluoroethyl)-1H-indol-4-amine |
| 611. | 3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propane-1,2-diol |
| 612. | 4-[(2-{3-[(2,4-dimethoxyphenyl)-amino]prop-1-yn-1-yl}-1-(2,2,2-trifluoroethyl)-1H- |
| | indol-4-yl)amino]-1λ⁶-thiane-1,1-dione |
| 613. | methyl 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxybenzoate |
| 614. | 2-{3-[(4-methanesulfonyl-2-methoxyphenyl)amino]prop-1-yn-1-yl}-N-[1-(2- |
| | methanesulfonylethyl)-piperidin-4-yl]-1-(2,2,2-trifluoroethyl)-1H-indol-4-amine |
| 615. | 2-{3-[(4-methanesulfonyl-2-methoxyphenyl)amino]prop-1-yn-1-yl}-N-[1-(2- |
| | methoxyethyl)piperidin-4-yl]-1-(2,2,2-trifluoroethyl)-1H-indol-4-amine |
| 616. | (1S,4S)-4-N-(2-{3-[(4-methanesulfonyl-2-methoxyphenyl)amino]prop-1-yn-1-yl}-1- |
| | (2,2,2-trifluoroethyl)-1H-indol-4-yl)-1-N,1-N-dimethylcyclohexane-1,4-diamine |
| 617. | (1R,4R)-4-N-(2-{3-[(4-methanesulfonyl-2-methoxyphenyl)amino]prop-1-yn-1-yl}-1- |
| | (2,2,2-trifluoroethyl)-1H-indol-4-yl)-1-N,1-N-dimethylcyclohexane-1,4-diamine |
| 618. | 2-{3-[(4-methanesulfonyl-2-methoxyphenyl)amino]prop-1-yn-1-yl}-N-[1-(oxan-4- |
| | yl)piperidin-4-yl]-1-(2,2,2-trifluoroethyl)-1H-indol-4-amine |
| 619. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-o |
| 620. | 2-{3-[(4-methanesulfonyl-2-methoxyphenyl)amino]prop-1-yn-1-yl}-N-[1-(1- |
| | methylpyrrolidin-3-yl)piperidin-4-yl]-1-(2,2,2-trifluoroethyl)-1H-indol-4-amine |
| 621. | 2-{3-[(4-methanesulfonyl-2-methoxyphenyl)amino]prop-1-yn-1-yl}-N-(2- |
| | methylpiperidin-4-yl)-1-(2,2,2-trifluoroethyl)-1H-indol-4-amine |
| 622. | 2-{3-[(4-methanesulfonyl-2-methoxyphenyl)amino]prop-1-yn-1-yl}-N-(piperidin-4-yl)-1- |
| | (2,2,2-trifluoroethyl)-1H-indol-4-amine |
| 623. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-1-(4-methylpiperazin-1-yl)ethan-1-one |
| 624. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}acetamide |
| 625. | 2-{3-[(4-methanesulfonyl-2-methoxyphenyl)amino]prop-1-yn-1-yl}-N-[1-(1- |
| | methylpiperidin-4-yl)piperidin-4-yl]-1-(2,2,2-trifluoroethyl)-1H-indol-4-amine |
| 626. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexyl}piperidin-4-ol |
| 627. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-1-(morpholin-4-yl)ethan-1-one |
| 628. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-N,N-dimethylacetamide |
| 629. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}acetonitrile |
| 630. | methyl 2-{4-[(2-{3-[(4-methane-sulfonyl-2-methoxyphenyl)amino]prop-1-yn-1-yl}-1- |
| | (2,2,2-trifluoroethyl)-1H-indol-4-yl)amino]piperidin-1-yl}acetate |
| 631. | 1-(4-hydroxypiperidin-1-yl)-2-{4-[(2-{3-[(4-methanesulfonyl-2-methoxy- |
| | phenyl)amino]prop-1-yn-1-yl}-1-(2,2,2-trifluoroethyl)-1H-indol-4-yl)amino]piperidin-1- |
| | yl}ethan-1-one |
| 632. | 2-(2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethoxy)ethan-1-ol |
| 633. | -[(1R,4R)-4-[(2-{3-[(4-methane-sulfonyl-2-methoxyphenyl)amino]prop-1-yn-1-yl}-1- |
| | (2,2,2-trifluoroethyl)-1H-indol-4-yl)amino]cyclohexyl]piperidin-4-ol |
| 634. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}acetic acid |
| 635. | (1R,4R)-4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-ol |
| 636. | (1S,4S)-4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-ol |
| 637. | 2-{3-[(4-methanesulfonyl-2-methoxyphenyl)amino]prop-1-yn-1-yl}-1-(2,2,2- |
| | trifluoroethyl)-1H-indol-4-amine |
| 638. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexyl}-3-methylpyrrolidin-3-ol |
| 639. | (3R,4R)-1-{4-[(2-{3-[(4-methane-sulfonyl-2-methoxyphenyl)amino]prop-1-yn-1-yl}-1- |
| | (2,2,2-trifluoroethyl)-1H-indol-4-yl)amino]cyclohexyl}-pyrrolidine-3,4-diol |
| 640. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carboximidamide |
| 641. | 1-[(1S,4S)-4-[(2-{3-[(4-methane-sulfonyl-2-methoxyphenyl)amino]prop-1-yn-1-yl}-1- |
| | (2,2,2-trifluoroethyl)-1H-indol-4-yl)amino]cyclohexyl]piperidin-4-ol |
| 642. | 4-[(2-{3-[(3-methoxypyridin-4-yl)amino]prop-1-yn-1-yl}-1-(2,2,2-trifluoroethyl)-1H- |
| | indol-4-yl)amino]-1λ⁶-thiane-1,1-dione |
| 643. | 2-{3-[(4-methanesulfonyl-2-methoxyphenyl)amino]prop-1-yn-1-yl}-N-[(1S,4S)-4- |
| | (morpholin-4-yl)cyclohexyl]-1-(2,2,2-trifluoroethyl)-1H-indol-4-amine |
| 644. | 2-{3-[(4-methanesulfonyl-2-methoxyphenyl)amino]prop-1-yn-1-yl}-N-[(1R,4R)-4- |
| | (morpholin-4-yl)cyclohexyl]-1-(2,2,2-trifluoroethyl)-1H-indol-4-amine |
| 645. | 2-(3-{[2-(2-fluoroethoxy)-4-methanesulfonylphenyl]amino}prop-1-yn-1-yl)-N-[1-(oxan- |
| | 4-yl)piperidin-4-yl]-1-(2,2,2-trifluoroethyl)-1H-indol-4-amine |
| 646. | 4-{[2-(3-{[4-methanesulfonyl-2-(trifluoromethyl)phenyl]amino}prop-1-yn-1-yl)-1-(2,2,2- |
| | trifluoroethyl)-1H-indol-4-yl]amino}-1λ⁶-thiane-1,1-dione |
| 647. | 2-(3-{[4-methanesulfonyl-2-(trifluoromethyl)phenyl]amino}prop-1-yn-1-yl)-N-(1- |
| | methylpiperidin-4-yl)-1-(2,2,2-trifluoroethyl)-1H-indol-4-amine |
| 648. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethan-1-one |
| 649. | 3-methoxy-4-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}benzene-1-sulfonamide |
| 650. | 3-methoxy-4-[(3-{4-[(piperidin-4-yl)amino]-1-(2,2,2-trifluoroethyl)-1H-indol-2-yl}prop- |
| | 2-yn-1-yl)amino]-benzamide |
| 651. | 3-methoxy-4-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}benzamide |
| 652. | 3-(4-{[2-(3-{[2-(2-fluoroethoxy)-4-methanesulfonylphenyl]amino}prop-1-yn-1-yl)-1- |
| | (2,2,2-trifluoroethyl)-1H-indol-4-yl]amino}piperidin-1-yl)propane-1,2-diol |
| 653. | 3-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)propane-1,2-diol |
| 654. | 2-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)acetamide |
| 655. | 2-(4-{[2-(3-{[2-(2-fluoroethoxy)-4-methanesulfonylphenyl]amino}prop-1-yn-1-yl)-1- |
| | (2,2,2-trifluoroethyl)-1H-indol-4-yl]amino}piperidin-1-yl)-1-(4-methylpiperazin-1- |
| | yl)ethan-1-one |
| 656. | 2-(3-{[2-(2-fluoroethoxy)-4-methanesulfonylphenyl]amino}prop-1-yn-1-yl)-N-(oxan-4- |
| | yl)-1-(2,2,2-trifluoroethyl)-1H-indol-4-amine |
| 657. | 2-(3-{[2-(2-fluoroethoxy)-4-methanesulfonylphenyl]amino}prop-1-yn-1-yl)-N-[1-(1- |
| | methylpiperidin-4-yl)piperidin-4-yl]-1-(2,2,2-trifluoroethyl)-1H-indol-4-amine |
| 658. | 4-[(2-{3-[(4-methoxypyridin-3-yl)amino]prop-1-yn-1-yl}-1-(2,2,2-trifluoroethyl)-1H- |
| | indol-4-yl)amino]-1λ⁶-thiane-1,1-dione |
| 659. | S-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}-2-hydroxyethane-1-sulfonamido |
| 660. | 2-hydroxy-S-{3-methoxy-4-[(3-{4-[(1-methylpiperidin-4-yl)amino]-1-(2,2,2- |
| | trifluoroethyl)-1H-indol-2-yl}prop-2-yn-1-yl)amino]phenyl}ethane-1-sulfonamido |
| 661. | 2-methyl-2-[5-({3-[4-(morpholin-4-yl)-1-(2,2,2-trifluoroethyl)-1H-indol-2-yl]prop-2-yn- |
| | 1-yl}amino)pyridin-2-yl]propanenitrile |
| 662. | -{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-N-[5-(2-{4-[(2-{3-[(4- |
| | methanesulfonylphenyl)amino]prop-1-yn-1-yl}-1-(2,2,2-trifluoroethyl)-1H-indol-4- |
| | yl)amino]piperidin-1-yl}acetamido)pentyl]acetamide |
| 663. | 6-[(2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}acetyl)oxy]hexyl 2-{4-[(2-{3-[(4- |
| | methanesulfonylphenyl)amino]prop-1-yn-1-yl}-1-(2,2,2-trifluoroethyl)-1H-indol-4- |
| | yl)amino]piperidin-1-yl}acetate |
| 664. | 3-methoxy-4-({3-[4-({1-[2-(4-methylpiperazin-1-yl)-2-oxoethyl]piperidin-4-yl}amino)-1- |
| | (2,2,2-trifluoroethyl)-1H-indol-2-yl]prop-2-yn-1-yl}amino)benzene-1-sulfonamide |
| 665. | 2-{5-methanesulfonyl-2-[(3-{4-[(1-methylpiperidin-4-yl)amino]-1-(2,2,2-trifluoroethyl)- |
| | 1H-indol-2-yl}prop-2-yn-1-yl)amino]phenoxy}acetamide |
| 666. | 2-(3-{[4-methanesulfonyl-2-(2-methoxyethoxy)phenyl]amino}prop-1-yn-1-yl)-N-[l- |
| | (oxan-4-yl)piperidin-4-yl]-1-(2,2,2-trifluoroethyl)-1H-indol-4-amine |
| 667. | 2-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)ethan-1-ol |
| 668. | 3-methoxy-4-[(3-{4-[(oxan-4-yl)amino]-1-(2,2,2-trifluoroethyl)-1H-indol-2-yl}prop-2- |
| | yn-1-yl)amino]benzoic acid |
| 669. | 2-{2-[(3-{4-[(1,1-dioxo-1λ⁶-thian-4-yl)amino]-1-(2,2,2-trifluoroethyl)-1H-indol-2- |
| | yl}prop-2-yn-1-yl)amino]-5-methanesulfonylphenoxy}acetamide |
| 670. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carboxamide |
| 671. | 2-{3-[(4-methoxypyridin-3-yl)amino]prop-1-yn-1-yl}-N-(1-methylpiperidin-4-yl)-1- |
| | (2,2,2-trifluoroethyl)-1H-indol-4-amine |
| 672. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carbothioamide |
| 673. | 4-[(2-{3-[(6-methanesulfonyl-4-methoxypyridin-3-yl)amino]prop-1-yn-1-yl}-1-(2,2,2- |
| | trifluoroethyl)-1H-indol-4-yl)amino]-1λ⁶-thiane-1,1-dione |
| 674. | 3-methoxy-4-{[3-(4-{[1-(2-methoxyethyl)piperidin-4-yl]amino}-1-(2,2,2-trifluoroethyl)- |
| | 1H-indol-2-yl)prop-2-yn-1-yl]amino}benzene-1-sulfonamide |
| 675. | 4-{[2-(3-{[4-(ethanesulfonyl)-2-methoxyphenyl]amino}prop-1-yn-1-yl)-1-(2,2,2- |
| | trifluoroethyl)-1H-indol-4-yl]amino}-1λ⁶-thiane-1,1-dione |
| 676. | 2-(3-{[4-(ethanesulfonyl)-2-methoxyphenyl]amino}prop-1-yn-1-yl)-N-[1-(oxan-4- |
| | yl)piperidin-4-yl]-1-(2,2,2-trifluoroethyl)-1H-indol-4-amine |
| 677. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxybenzoic acid |
| 678. | methyl 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxybenzoate |
| 679. | methyl 3-methoxy-4-[(3-{4-[(oxan-4-yl)amino]-1-(2,2,2-trifluoroethyl)-1H-indol-2- |
| | yl}prop-2-yn-1-yl)amino]benzoate |
| 680. | 3-methoxy-4-({3-[4-({1-[2-(4-methylpiperazin-1-yl)-2-oxoethyl]piperidin-4-yl}amino)-1- |
| | (2,2,2-trifluoroethyl)-1H-indol-2-yl]prop-2-yn-1-yl}amino)benzamide |
| 681. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]-N-methylpiperidine-1-carboximidamide |
| 682. | 2-{3-[(6-methanesulfonyl-4-methoxypyridin-3-yl)amino]prop-1-yn-1-yl}-N-(1- |
| | methylpiperidin-4-yl)-1-(2,2,2-trifluoroethyl)-1H-indol-4-amine |
| 683. | 2-{3-[(4-methanesulfonyl-2-methoxyphenyl)amino]prop-1-yn-1-yl}-N-[1-(pyridin-4- |
| | yl)piperidin-4-yl]-1-(2,2,2-trifluoroethyl)-1H-indol-4-amine |
| 684. | 3-(4-{[2-(3-{[4-(ethanesulfonyl)-2-methoxyphenyl]amino}prop-1-yn-1-yl)-1-(2,2,2- |
| | trifluoroethyl)-1H-indol-4-yl]amino}piperidin-1-yl)propane-1,2-diol |
| 685. | 2-(3-{[4-(ethanesulfonyl)-2-methoxyphenyl]amino}prop-1-yn-1-yl)-N-(1- |
| | methylpiperidin-4-yl)-1-(2,2,2-trifluoroethyl)-1H-indol-4-amine |
| 686. | 2-hydroxy-S-{3-methoxy-4-[(3-{4-[(oxan-4-yl)amino]-1-(2,2,2-trifluoroethyl)-1H-indol- |
| | 2-yl}prop-2-yn-1-yl)amino]phenyl}ethane-1-sulfonamido |
| 687. | 2-(3-{[4-methanesulfonyl-2-(2,2,2-trifluoroethoxy)phenyl]amino}prop-1-yn-1-yl)-N-(1- |
| | methylpiperidin-4-yl)-1-(2,2,2-trifluoroethyl)-1H-indol-4-amine |
| 688. | 4-{[2-(3-{[4-methanesulfonyl-2-(2,2,2-trifluoroethoxy)phenyl]amino}prop-1-yn-1-yl)-1- |
| | (2,2,2-trifluoroethyl)-1H-indol-4-yl]amino}-1λ⁶-thiane-1,1-dione |
| 689. | 2-hydroxy-S-(3-methoxy-4-{[3-(4-{[1-(oxan-4-yl)piperidin-4-yl]amino}-1-(2,2,2- |
| | trifluoroethyl)-1H-indol-2-yl)prop-2-yn-1-yl]amino}phenyl)ethane-1-sulfonamido |
| 690. | S-(4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxyphenyl)-2-hydroxyethane-1-sulfonamido |
| 691. | 2-{3-methoxy-4-[(3-{4-[(1-methylpiperidin-4-yl)amino]-1-(2,2,2-trifluoroethyl)-1H- |
| | indol-2-yl}prop-2-yn-1-yl)amino]phenyl}-2-methylpropanenitrile |
| 692. | 3-methoxy-4-{[3-(4-{[(1S,4S)-4-[(3R,4R)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 693. | 3-methoxy-4-{[3-(4-{[(1R,4R)-4-[(3R,4R)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 694. | 3-methoxy-4-{[3-(4-{[(1S,4S)-4-(4-hydroxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 695. | 3-methoxy-4-{[3-(4-{[(1R,4R)-4-(4-hydroxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 696. | 2-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}-2-methylpropanenitrile |
| 697. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 698. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 699. | (3S,4S)-1-[(1S,4S)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]pyrrolidine-3,4-diol |
| 700. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 701. | 3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 702. | 2-{3-[(4-methanesulfonyl-2-methoxyphenyl)amino]prop-1-yn-1-yl}-N,N-dimethyl-1- |
| | (2,2,2-trifluoroethyl)-1H-indol-4-amine |
| 703. | 3-methoxy-4-[(3-{4-[(2-methoxyethyl)amino]-1-(2,2,2-trifluoroethyl)-1H-indol-2- |
| | yl}prop-2-yn-1-yl)amino]benzene-1-sulfonamide |
| 704. | 2-[(2-{3-[(2-methoxy-4-sulfamoylphenyl)amino]prop-1-yn-1-yl}-1-(2,2,2-trifluoroethyl)- |
| | 1H-indol-4-yl)amino]acetamide |
| 705. | 4-[(3-{4-[(1,5-dihydroxypentan-3-yl)amino]-1-(2,2,2-trifluoroethyl)-1H-indol-2-yl}prop- |
| | 2-yn-1-yl)amino]-3-methoxybenzene-1-sulfonamide |
| 706. | N-(2-{3-[(4-methanesulfonyl-2-methoxyphenyl)amino]prop-1-yn-1-yl}-1-(2,2,2- |
| | trifluoroethyl)-1H-indol-4-yl)acetamide |
| 707. | 3-(2-{3-[(4-methanesulfonyl-2-methoxyphenyl)amino]prop-1-yn-1-yl}-1-(2,2,2- |
| | trifluoroethyl)-1H-indol-4-yl)-1-(1-methylpiperidin-4-yl)urea |
| 708. | 3-methoxy-4-{[3-(4-{[(1-methylpiperidin-4-yl)carbamoyl]amino}-1-(2,2,2- |
| | trifluoroethyl)-1H-indol-2-yl)prop-2-yn-1-yl]amino}benzamide |
| 709. | N-(2-{3-[(4-methanesulfonyl-2-methoxyphenyl)amino]prop-1-yn-1-yl}-1-(2,2,2- |
| | trifluoroethyl)-1H-indol-4-yl)-4-methylpiperazine-1-carboxamide |
| 710. | N-(2-{3-[(4-carbamoyl-2-methoxyphenyl)amino]prop-1-yn-1-yl}-1-(2,2,2-trifluoroethyl)- |
| | 1H-indol-4-yl)-4-methylpiperazine-1-carboxamide |
| 711. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[(1S,4S)-4-(dimethylamino)cyclohexyl]urea |
| 712. | 1-(2-(3-((2-methoxy-4-(methylsulfonyl)phenyl)amino)prop-1-yn-1-yl)-1-(2,2,2- |
| | trifluoroethyl)-1H-indol-4-yl)-3-(pyridin-4-yl)urea |
| 713. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[(1R,4R)-4-(dimethylamino)cyclohexyl]urea |
| 714. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[1-(2-methoxyethyl)piperidin-4-yl]urea |
| 715. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[1-(oxan-4-yl)piperidin-4-yl]urea |
| 716. | 1-[1-(2,3-dihydroxypropyl)piperidin-4-yl]-3-(2-{3-[(4-methanesulfonyl-2- |
| | methoxyphenyl)amino]prop-1-yn-1-yl}-1-(2,2,2-trifluoroethyl)-1H-indol-4-yl)urea |
| 717. | 2-(4-{[(2-{3-[(4-methanesulfonyl-2-methoxyphenyl)amino]prop-1-yn-1-yl}-1-(2,2,2- |
| | trifluoroethyl)-1H-indol-4-yl)carbamoyl]amino}piperidin-1-yl)acetamide |
| 718. | 3-methoxy-4-({3-[4-({2-oxaspiro[3.3]heptan-6-yl}amino)-1-(2,2,2-trifluoroethyl)-1H- |
| | indol-2-yl]prop-2-yn-1-yl}amino)benzene-1-sulfonamide |
| 719. | [1-(chloromethyl)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclobutyl]methanol |
| 720. | 2-{3-[(4-methanesulfonyl-2-methoxyphenyl)amino]prop-1-yn-1-yl}-N-{2- |
| | oxaspiro[3.3]heptan-6-yl}-1-(2,2,2-trifluoroethyl)-1H-indol-4-amine |
| 721. | 4-({3-[4-({2-azaspiro[3.3]heptan-6-yl}amino)-1-(2,2,2-trifluoroethyl)-1H-indol-2- |
| | yl]prop-2-yn-1-yl}amino)-3-methoxybenzene-1-sulfonamide |
| 722. | N-{2-azaspiro[3.3]heptan-6-yl}-2-{3-[(4-methanesulfonyl-2-methoxyphenyl)amino]prop- |
| | 1-yn-1-yl}-1-(2,2,2-trifluoroethyl)-1H-indol-4-amine |
| 723. | 3-methoxy-N-methyl-4-{[3-(4-{[(1R,4R)-4-(methylamino)cyclohexyl]amino}-1-(2,2,2- |
| | trifluoroethyl)-1H-indol-2-yl)prop-2-yn-1-yl]amino}benzamide |
| 724. | 3-methoxy-N-methyl-4-{[3-(4-{[(1R,4R)-4-aminocyclohexyl]amino}-1-(2,2,2- |
| | trifluoroethyl)-1H-indol-2-yl)prop-2-yn-1-yl]amino}benzamide |
| 725. | 3-methoxy-N-methyl-4-{[3-(4-{[(1S,4S)-4-aminocyclohexyl]amino}-1-(2,2,2- |
| | trifluoroethyl)-1H-indol-2-yl)prop-2-yn-1-yl]amino}benzamide |
| 726. | re1-(1R,3R)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 727. | rac-(1R,3S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 728. | (1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamineQ |
| 729. | rac-(1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamine |
| 730. | re1-(1R,3S)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexan-1-ol |
| 731. | 3-methoxy-N-methyl-4-{[3-(4-{[(1R,4R)-4-hydroxy-4-methylcyclohexyl]amino}-1- |
| | (2,2,2-trifluoroethyl)-1H-indol-2-yl)prop-2-yn-1-yl]amino}benzamide |
| 732. | 3-methoxy-N-methyl-4-{[3-(4-{[(1S,4S)-4-hydroxy-4-methylcyclohexyl]amino}-1- |
| | (2,2,2-trifluoroethyl)-1H-indol-2-yl)prop-2-yn-1-yl]amino}benzamide |
| 733. | 4-[(3-{4-[(4-cyanocyclohexyl)amino]-1-(2,2,2-trifluoroethyl)-1H-indol-2-yl}prop-2-yn-1- |
| | yl)amino]-3-methoxy-N-methylbenzamide |
| 734. | 3-methoxy-N-methyl-4-{[3-(4-{[(1R,4R)-4-cyanocyclohexyl]amino}-1-(2,2,2- |
| | trifluoroethyl)-1H-indol-2-yl)prop-2-yn-1-yl]amino}benzamide |
| 735. | 3-methoxy-N-methyl-4-{[3-(4-{[(1S,4S)-4-cyanocyclohexyl]amino}-1-(2,2,2- |
| | trifluoroethyl)-1H-indol-2-yl)prop-2-yn-1-yl]amino}benzamide |
| 736. | 3-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexane-1-carboxylic acid |
| 737. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)cyclohexane-1,4-diamine |
| 738. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)cyclohexane-1,4-diamine |
| 739. | 2-fluoro-N¹-(2-{3-[(4-methanesulfonyl-2-methoxyphenyl)amino]prop-1-yn-1-yl}-1- |
| | (2,2,2-trifluoroethyl)-1H-indol-4-yl)-N⁴-methylcyclohexane-1,4-diamine |
| 740. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴-methylcyclohexane-1,4-diamine |
| 741. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 742. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴,N⁴-dimethylcyclohexane-1,4-diamine |
| 743. | (3S,4R)-3-fluoro-4-[(2-{3-[(4-methanesulfonyl-2-methoxyphenyl)amino]prop-1-yn-1-yl}- |
| | 1-(2,2,2-trifluoroethyl)-1H-indol-4-yl)amino]cyclohexan-1-ol |
| 744. | 3-methoxy-4-{[3-(4-{[(1R,4R)-4-[bis(2-hydroxyethyl)amino]cyclohexyl]amino}-1- |
| | (2,2,2-trifluoroethyl)-1H-indol-2-yl)prop-2-yn-1-yl]amino}benzene-1-sulfonamide |
| 745. | 3-methoxy-4-{[3-(4-{[(1S,4S)-4-[bis(2-hydroxyethyl)amino]cyclohexyl]amino}-1-(2,2,2- |
| | trifluoroethyl)-1H-indol-2-yl)prop-2-yn-1-yl]amino}benzene-1-sulfonamide |
| 746. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 747. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 748. | 3-methoxy-N-(1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 749. | 3-methoxy-N-(oxan-4-yl)-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 750. | 3-methoxy-N-(1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 751. | 3-methoxy-N-(oxan-4-yl)-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 752. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 753. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 754. | 3-methoxy-N-(5-methyl-1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 755. | 3-methoxy-N-(5-methyl-1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 756. | (1S,4S)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 757. | (1R,4R)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 758. | 3-methoxy-4-{[3-(4-{[(1S,4S)-4-[(3S,4S)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 759. | 4-((3-(4-(((1R,4R)-4-((3R,4R)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 760. | 4-((3-(4-(((1R,4S)-4-((3S,4S)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 761. | 3-methoxy-4-{[3-(4-{[(1R,4R)-4-[bis(2-methoxyethyl)amino]cyclohexyl]amino}-1- |
| | (2,2,2-trifluoroethyl)-1H-indol-2-yl)prop-2-yn-1-yl]amino}benzene-1-sulfonamide |
| 762. | 3-methoxy-4-{[3-(4-{[(1S,4S)-4-[bis(2-methoxyethyl)amino]cyclohexyl]amino}-1-(2,2,2- |
| | trifluoroethyl)-1H-indol-2-yl)prop-2-yn-1-yl]amino}benzene-1-sulfonamide |
| 763. | 4-({3-[4-({1,4-dioxaspiro[4.5]decan-8-yl}amino)-1-(2,2,2-trifluoroethyl)-1H-indol-2- |
| | yl]prop-2-yn-1-yl}amino)-3-methoxybenzene-1-sulfonamide |
| 764. | N-{1,4-dioxaspiro[4.5]decan-8-yl}-2-{3-[(4-methanesulfonyl-2- |
| | methoxyphenyl)amino]prop-1-yn-1-yl}-1-(2,2,2-trifluoroethyl)-1H-indol-4-amine |
| 765. | 3-methoxy-4-[(3-{4-[(4-oxocyclohexyl)amino]-1-(2,2,2-trifluoroethyl)-1H-indol-2- |
| | yl}prop-2-yn-1-yl)amino]benzene-1-sulfonamide |
| 766. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-one |
| 767. | (1R,4R)-N⁴-[2-(3-{[2-methoxy-4-(morpholine-4-sulfonyl)phenyl]amino}prop-1-yn-1- |
| | yl)-1-(2,2,2-trifluoroethyl)-1H-indol-4-yl]-N¹,N¹-dimethylcyclohexane-1,4-diamine |
| 768. | (1S,4S)-N⁴-[2-(3-{[2-methoxy-4-(morpholine-4-sulfonyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 769. | N,N-bis(2-hydroxyethyl)-3-methoxy-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 770. | N,N-bis(2-hydroxyethyl)-3-methoxy-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 771. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 772. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 773. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 774. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 775. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 776. | (1R,4R)-N⁴-{2-[3-({2-methoxy-4-[(4-methylpiperazin-1-yl)sulfonyl]phenyl}amino)prop- |
| | 1-yn-1-yl]-1-(2,2,2-trifluoroethyl)-1H-indol-4-yl}-N¹,N¹-dimethylcyclohexane-1,4- |
| | diamine |
| 777. | (1R,4R)-N⁴-(2-{3-[(4-methanesulfonyl-2-methoxyphenyl)amino]prop-1-yn-1-yl}-1- |
| | [(oxiran-2-yl)methyl]-1H-indol-4-yl)-N¹,N¹-dimethylcyclohexane-1,4-diamine |
| 778. | 2-[(2-{3-[(4-methanesulfonyl-2-methoxyphenyl)amino]prop-1-yn-1-yl}-4-{[(1R,4R)-4- |
| | (dimethylamino)cyclohexyl]amino}-1H-indol-1-yl)methyl]prop-2-enenitrile |
| 779. | 5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenol |
| 780. | 5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenol |
| 781. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 782. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 783. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 784. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 785. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 786. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 787. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 788. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 789. | (1R,4R)-N⁴-[2-(3-{[4-methanesulfonyl-2-(2-methoxyethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 790. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 791. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 792. | (1R,4R)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 793. | (1S,4S)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 794. | (1S,4S)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 795. | (1R,4R)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 796. | (1S,4S)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 797. | 3-hydroxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 798. | 3-hydroxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 799. | 3-(2-methoxyethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzamide |
| 800. | (1R,4R)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 801. | N-ethyl-3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 802. | N-ethyl-3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 803. | 3-(2-methoxyethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzamide |
| 804. | 3-(2-fluoroethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 805. | 3-(2-fluoroethoxy)-N-methyl-4-{[3-(4-{[(1S,4S)-4-(dimethylamino)cyclohexyl]amino}- |
| | 1-(2,2,2-trifluoroethyl)-1H-indol-2-yl)prop-2-yn-1-yl]amino}benzamide |
| 806. | 3-(fluoromethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 807. | 3-(fluoromethoxy)-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 808. | (1S,4S)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 809. | (1R,4R)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 810. | 3-methoxy-N-methyl-4-{[3-(4-{[(1S,4S)-4-(methylamino)cyclohexyl]amino}-1-(2,2,2- |
| | trifluoroethyl)-1H-indol-2-yl)prop-2-yn-1-yl]amino}benzamide |
| 811. | (1S,4S)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 812. | (1R,4R)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 813. | 3-(cyanomethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 814. | 3-(2-cyanoethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 815. | N-(5-methanesulfonyl-2-{[3-(4-{[(1R,4R)-4-(dimethylamino)cyclohexyl]amino}-1- |
| | (2,2,2-trifluoroethyl)-1H-indol-2-yl)prop-2-yn-1-yl]oxy}phenyl)acetamide |
| 816. | N-(2-((3-(4-(((1S,4S)-4-(dimethylamino)cyclohexyl)amino)-1-(2,2,2-trifluoroethyl)-1H- |
| | indol-2-yl)prop-2-yn-1-yl)oxy)-5-(methylsulfonyl)phenyl)acetamide |
| 817. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 818. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 819. | (1R,4R)-N⁴-[2-(3-{[4-(ethanesulfonyl)-2-methoxyphenyl]amino}prop-1-yn-1-yl)-1- |
| | (2,2,2-trifluoroethyl)-1H-indol-4-yl]-N¹,N¹-dimethylcyclohexane-1,4-diamine |
| 820. | (1S,4S)-N⁴-[2-(3-{[4-(ethanesulfonyl)-2-methoxyphenyl]amino}prop-1-yn-1-yl)-1-(2,2,2- |
| | trifluoroethyl)-1H-indol-4-yl]-N¹,N¹-dimethylcyclohexane-1,4-diamine |
| 821. | 2-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenyl)-2-methylpropanenitrile |
| 822. | 2-(4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxyphenyl)-2-methylpropanenitrile |
| 823. | 3-(cyanomethoxy)-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 824. | 3-(cyanomethoxy)-4-((3-(4-(((1S,4S)-4-(dimethylamino)cyclohexyl)amino)-1-(2,2,2- |
| | trifluoroethyl)-1H-indol-2-yl)prop-2-yn-1-yl)amino)benzenesulfonamide |
| 825. | 3-(3-(4-(((1R,4R)-4-(dimethylamino)cyclohexyl)amino)-1-(2,2,2-trifluoroethyl)-1H- |
| | indol-2-yl)prop-2-yn-1-yl)-6-(methylsulfonyl)benzo[d]oxazol-2(3H)-one |
| 826. | 3-(3-(4-(((1S,4S)-4-(dimethylamino)cyclohexyl)amino)-1-(2,2,2-trifluoroethyl)-1H-indol- |
| | 2-yl)prop-2-yn-1-yl)-6-(methylsulfonyl)benzo[d]oxazol-2(3H)-one |
| 827. | 3-methoxy-N,N-dimethyl-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 828. | (1R,4R)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 829. | (1S,4S)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 830. | (1R,4R)-N⁴-(2-{3-[(4-chloro-2-methoxyphenyl)amino]prop-1-yn-1-yl}-1-(2,2,2- |
| | trifluoroethyl)-1H-indol-4-yl)-N¹,N¹-dimethylcyclohexane-1,4-diamine |
| 831. | (1S,4S)-N¹-(2-(3-((4-chloro-2-methoxyphenyl)amino)prop-1-yn-1-yl)-1-(2,2,2- |
| | trifluoroethyl)-1H-indol-4-yl)-N⁴,N⁴-dimethylcyclohexane-1,4-diamine |
| 832. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 833. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 834. | (1R,4R)-N⁴-[2-(3-{[2-methoxy-4-(trifluoromethyl)phenyl]amino}prop-1-yn-1-yl)-1- |
| | (2,2,2-trifluoroethyl)-1H-indol-4-yl]-N¹,N¹-dimethylcyclohexane-1,4-diamine |
| 835. | (1S,4S)-N¹-(2-(3-((2-methoxy-4-(trifluoromethyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 836. | 2-fluoro-5-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 837. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxy-N-methylbenzamide |
| 838. | (1S,4S)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 839. | (1R,4R)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 840. | (1R,4R)-N⁴-(2-{3-[(4-methanesulfonyl-2-methylphenyl)amino]prop-1-yn-1-yl}-1-(2,2,2- |
| | trifluoroethyl)-1H-indol-4-yl)-N¹,N¹-dimethylcyclohexane-1,4-diamine |
| 841. | (1S,4S)-N¹,N¹-dimethyl-N⁴-(2-(3-((2-methyl-4-(methylsulfonyl)phenyl)amino)prop-1-yn- |
| | 1-yl)-1-(2,2,2-trifluoroethyl)-1H-indol-4-yl)cyclohexane-1,4-diamine |
| 842. | (1R,4R)-N⁴-[2-(3-{[4-methanesulfonyl-2-(trifluoromethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 843. | (1S,4S)-N¹,N¹-dimethyl-N⁴-(2-(3-((4-(methylsulfonyl)-2- |
| | (trifluoromethoxy)phenyl)amino)prop-1-yn-1-yl)-1-(2,2,2-trifluoroethyl)-1H-indol-4- |
| | yl)cyclohexane-1,4-diamine |
| 844. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzamide |
| 845. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxy-N-methylbenzamide |
| 846. | 3-methoxy-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 847. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxybenzenesulfonamide |
| 848. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 849. | 2-(5-methanesulfonyl-2-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 850. | 2-(5-methanesulfonyl-2-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 851. | 3-methoxy-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 852. | 3-methoxy-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 853. | N-ethyl-3-methoxy-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 854. | N-ethyl-3-methoxy-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)benzamide |
| 855. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 856. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 857. | 3-(fluoromethoxy)-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 858. | 3-(fluoromethoxy)-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 859. | 3-(fluoromethoxy)-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)-N-methylbenzamide |
| 860. | 3-methoxy-4-{[3-(4-{[(1R,4R)-4-[(2-methoxyethyl)(methyl)amino]cyclohexyl]amino}-1- |
| | (2,2,2-trifluoroethyl)-1H-indol-2-yl)prop-2-yn-1-yl]amino}benzene-1-sulfonamide |
| 861. | 3-methoxy-4-((3-(4-(((1S,4S)-4-((2-methoxyethyl)(methyl)amino)cyclohexyl)amino)-1- |
| | (2,2,2-trifluoroethyl)-1H-indol-2-yl)prop-2-yn-1-yl)amino)benzenesulfonamide |
| 862. | (1R,4R)-N¹,N¹-diethyl-N⁴-(2-{3-[(4-methanesulfonyl-2-methoxyphenyl)amino]prop-1- |
| | yn-1-yl}-1-(2,2,2-trifluoroethyl)-1H-indol-4-yl)cyclohexane-1,4-diamine |
| 863. | (1S,4S)-N¹,N¹-diethyl-N⁴-(2-(3-((2-methoxy-4-(methylsulfonyl)phenyl)amino)prop-1-yn- |
| | 1-yl)-1-(2,2,2-trifluoroethyl)-1H-indol-4-yl)cyclohexane-1,4-diamine |
| 864. | 3-(cyanomethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 865. | 3-(cyanomethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 866. | 3-(fluoromethoxy)-4-{[3-(4-{[(1R,4R)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 867. | 3-(fluoromethoxy)-4-{[3-(4-{[(1S,4S)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 868. | 3-(fluoromethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 869. | 3-(fluoromethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 870. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 871. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 872. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 873. | 3-methoxy-N-methyl-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 874. | 2-(2-{3-[(4-methanesulfonyl-2-methoxyphenyl)amino]prop-1-yn-1-yl}-4-{[(1R,4R)-4- |
| | (morpholin-4-yl)cyclohexyl]amino}-1H-indol-1-yl)acetonitrile |
| 875. | 4-({3-[1-(2-fluoroethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 876. | 1-(2-fluoroethyl)-2-{3-[(4-methanesulfonyl-2-methoxyphenyl)amino]prop-1-yn-1-yl}-N- |
| | [(1R,4R)-4-(morpholin-4-yl)cyclohexyl]-1H-indol-4-amine |
| 877. | 4-({3-[1-(cyanomethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 878. | 2-{3-[(4-methanesulfonyl-2-methoxyphenyl)amino]prop-1-yn-1-yl}-1-propyl-N- |
| | [(1R,4R)-4-(morpholin-4-yl)cyclohexyl]-1H-indol-4-amine |
| 879. | 2-{3-[(4-methanesulfonyl-2-methoxyphenyl)amino]prop-1-yn-1-yl}-1-(2-methylpropyl)- |
| | N-[(1R,4R)-4-(morpholin-4-yl)cyclohexyl]-1H-indol-4-amine |
| 880. | 1-(2,2-difluoroethyl)-2-{3-[(4-methanesulfonyl-2-methoxyphenyl)amino]prop-1-yn-1- |
| | yl}-N-[(1R,4R)-4-(morpholin-4-yl)cyclohexyl]-1H-indol-4-amine |
| 881. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 882. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 883. | N-ethyl-3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 884. | N-ethyl-3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzamide |
| 885. | 2-(5-methanesulfonyl-2-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 886. | 2-(5-methanesulfonyl-2-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 887. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 888. | 2-{3-[(4-methanesulfonyl-2-methoxyphenyl)amino]prop-1-yn-1-yl}-N-[(1R,4R)-4-(3- |
| | methanesulfonylazetidin-1-yl)cyclohexyl]-1-(2,2,2-trifluoroethyl)-1H-indol-4-amine |
| 889. | 2-(3-((2-methoxy-4-(methylsulfonyl)phenyl)amino)prop-1-yn-1-yl)-N-((1S,4S)-4-(3- |
| | (methylsulfonyl)azetidin-1-yl)cyclohexyl)-1-(2,2,2-trifluoroethyl)-1H-indol-4-amine |
| 890. | 3-methoxy-4-{[3-(4-{[(1R,4R)-4-(3-methanesulfonylazetidin-1-yl)cyclohexyl]amino}-1- |
| | (2,2,2-trifluoroethyl)-1H-indol-2-yl)prop-2-yn-1-yl]amino}benzene-1-sulfonamide |
| 891. | 3-methoxy-4-((3-(4-(((1S,4S)-4-(3-(methylsulfonyl)azetidin-1-yl)cyclohexyl)amino)-1- |
| | (2,2,2-trifluoroethyl)-1H-indol-2-yl)prop-2-yn-1-yl)amino)benzenesulfonamide |
| 892. | 3-methoxy-N-methyl-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzamide |
| 893. | 3-methoxy-N-methyl-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzamide |
| 894. | 2-{3-[(4-methanesulfonyl-2-methoxyphenyl)amino]prop-1-yn-1-yl}-N-[(1R,4R)-4-{2- |
| | azaspiro[3.3]heptan-2-yl}cyclohexyl]-1-(2,2,2-trifluoroethyl)-1H-indol-4-amine |
| 895. | N-((1S,4S)-4-(2-azaspiro[3.3]heptan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 896. | 3-methoxy-N-methyl-4-{[3-(4-{[(1R,4R)-4-{2-azaspiro[3.3]heptan-2- |
| | yl}cyclohexyl]amino}-1-(2,2,2-trifluoroethyl)-1H-indol-2-yl)prop-2-yn-1- |
| | yl]amino}benzamide |
| 897. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 898. | 3-methoxy-4-{[3-(4-{[(1R,4R)-4-{2-azaspiro[3.3]heptan-2-yl}cyclohexyl]amino}-1- |
| | (2,2,2-trifluoroethyl)-1H-indol-2-yl)prop-2-yn-1-yl]amino}benzene-1-sulfonamide |
| 899. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxybenzenesulfonamide |
| 900. | 2-(3-((2-methoxy-4-(methylsulfonyl)phenyl)amino)prop-1-yn-1-yl)-N-(2- |
| | azaspiro[3.3]heptan-6-yl)-1-(2,2,2-trifluoroethyl)-1H-indol-4-amine |
| 901. | 4-((3-(4-((2-azaspiro[3.3]heptan-6-yl)amino)-1-(2,2,2-trifluoroethyl)-1H-indol-2-yl)prop- |
| | 2-yn-1-yl)amino)-3-methoxybenzenesulfonamide |
| 902. | 2-{3-[(4-methanesulfonyl-2-methoxyphenyl)amino]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 903. | 2-{3-[(4-methanesulfonyl-2-methoxyphenyl)amino]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 904. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 905. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 906. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 907. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 908. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 909. | N-(3-methoxy-4-{[3-(4-{[(1S,4S)-4-}2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 910. | N-((1R,4R)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 911. | N-((1S,4S)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 912. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 913. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 914. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 915. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 916. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 917. | N-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 918. | N-(2-hydroxy-4-methanesulfonylphenyl)-2-methyl-N-[3-(4-{[(1R,4R)-4-{2-oxa-6- |
| | azaspiro[3.3]heptan-6-yl}cyclohexyl]amino}-1-(2,2,2-trifluoroethyl)-1H-indol-2-yl)prop- |
| | 2-yn-1-yl]propanamide |
| 919. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)isobutyramide |
| 920. | 2-(3-{[4-methanesulfonyl-2-(2-methoxyethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 921. | 2-(3-{[4-methanesulfonyl-2-(2-methoxyethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 922. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 923. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 924. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 925. | 2-(5-methanesulfonyl-2-([3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 926. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 927. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)acetamide |
| 928. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 929. | 3-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 930. | 3-methoxy-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 931. | 2-(3-{[4-methanesulfonyl-2-(2,2,2-trifluoroethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 932. | 2-(3-{[4-methanesulfonyl-2-(2,2,2-trifluoroethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 933. | 2-[3-(2-amino-4-methanesulfonylphenoxy)prop-1-yn-1-yl]-N-[(1S,4S)-4-{2-oxa-6- |
| | azaspiro[3.3]heptan-6-yl}cyclohexyl]-1-(2,2,2-trifluoroethyl)-1H-indol-4-amine |
| 934. | 2-{3-[(2-ethoxy-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 935. | 2-{3-[(2-ethoxy-4-methanesulfonylphenyl)amino]prop-1-yn-1-yl}-N-[(1S,4S)-4-{2-oxa- |
| | 6-azaspiro[3.3]heptan-6-yl}cyclohexyl]-1-(2,2,2-trifluoroethyl)-1H-indol-4-amine |
| 936. | 3-(2-methoxyethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 937. | 3-(2-methoxyethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 938. | N-ethyl-3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 939. | N-ethyl-3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 940. | 3-(2-fluoroethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 941. | 3-(2-fluoroethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 942. | 3-(cyanomethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 943. | 3-(cyanomethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 944. | 2-{3-[4-methanesulfonyl-2-(methylamino)phenoxy]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 945. | 2-{3-[4-methanesulfonyl-2-(methylamino)phenoxy]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 946. | 2-[3-(2-amino-4-methanesulfonylphenoxy)prop-1-yn-1-yl]-N-[(1R,4R)-4-{2-oxa-6- |
| | azaspiro[3.3]heptan-6-yl}cyclohexyl]-1-(2,2,2-trifluoroethyl)-1H-indol-4-amine |
| 947. | N-((1R,4R)-4-(2-oxa-6-azaspiro[3.3]heptan-6-yl)cyclohexyl)-2-(3-(2-amino-4- |
| | (methylsulfonyl)phenoxy)prop-1-yn-1-yl)-1-(2,2,2-trifluoroethyl)-1H-indol-4-amine |
| 948. | 3-hydroxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 949. | 3-methoxy-4-[(3-{4-[(4-{2-oxa-6-azaspiro[3.3]heptan-6-yl}cyclohexyl)amino]-1-(2,2,2- |
| | trifluoroethyl)-1H-indol-2-yl}prop-2-yn-1-yl)amino]benzonitrile |
| 950. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzonitrile |
| 951. | 2-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 952. | 2-(4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 953. | 3-(fluoromethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 954. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(fluoromethoxy)-N- |
| | methylbenzamide |
| 955. | 3-(2-cyanoethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 956. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(2-cyanoethoxy)-N- |
| | methylbenzamide |
| 957. | 2-(3-{[2-(fluoromethoxy)-4-methanesulfonylphenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 958. | N-((1S,4S)-4-(2-oxa-6-azaspiro[3.3]heptan-6-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 959. | 3-(cyanomethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 960. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3- |
| | (cyanomethoxy)benzenesulfonamide |
| 961. | 3-(fluoromethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 962. | 3-(fluoromethoxy)-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 963. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 964. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 965. | N-((1R,4R)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 966. | N-((1S,4S)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 967. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 968. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 969. | N-((1R,4R)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 970. | N-((1S,4S)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 971. | N-((1R,4R)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 972. | N-((1S,4S)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 973. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 974. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzamide |
| 975. | 2-fluoro-5-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 976. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxy-N- |
| | methylbenzamide |
| 977. | 2-(3-{[2-methoxy-4-(trifluoromethyl)phenyl]amino}prop-1-yn-1-yl)-N-[(1R,4R)-4-{2- |
| | oxa-6-azaspiro[3.3]heptan-6-yl}cyclohexyl]-1-(2,2,2-trifluoroethyl)-1H-indol-4-amine |
| 978. | N-((1S,4S)-4-(2-oxa-6-azaspiro[3.3]heptan-6-yl)cyclohexyl)-2-(3-((2-methoxy-4- |
| | (trifluoromethyl)phenyl)amino)prop-1-yn-1-yl)-1-(2,2,2-trifluoroethyl)-1H-indol-4-amine |
| 979. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 980. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 981. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzoic acid |
| 982. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzoic acid |
| 983. | 2-{3-[(4-methanesulfonyl-2-methylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 984. | N-((1S,4S)-4-(2-oxa-6-azaspiro[3.3]heptan-6-yl)cyclohexyl)-2-(3-((2-methyl-4- |
| | (methylsulfonyl)phenyl)amino)prop-1-yn-1-yl)-1-(2,2,2-trifluoroethyl)-1H-indol-4-amine |
| 985. | 2-(3-{[4-methanesulfonyl-2-(trifluoromethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 986. | N-((1S,4S)-4-(2-oxa-6-azaspiro[3.3]heptan-6-yl)cyclohexyl)-2-(3-((4-(methylsulfonyl)-2- |
| | (trifluoromethoxy)phenyl)amino)prop-1-yn-1-yl)-1-(2,2,2-trifluoroethyl)-1H-indol-4- |
| | amine |
| 987. | 2-(3-{[4-methanesulfonyl-2-(trifluoromethyl)phenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 988. | N-((1S,4S)-4-(2-oxa-6-azaspiro[3.3]heptan-6-yl)cyclohexyl)-2-(3-((4-(methylsulfonyl)-2- |
| | (trifluoromethyl)phenyl)amino)prop-1-yn-1-yl)-1-(2,2,2-trifluoroethyl)-1H-indol-4-amine |
| 989. | 2-{3-[(2-chloro-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 990. | N-((1S,4S)-4-(2-oxa-6-azaspiro[3.3]heptan-6-yl)cyclohexyl)-2-(3-((2-chloro-4- |
| | (methylsulfonyl)phenyl)amino)prop-1-yn-1-yl)-1-(2,2,2-trifluoroethyl)-1H-indol-4-amine |
| 991. | 3-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 992. | 3-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 993. | 2-{3-[(4-methanesulfonyl-2-methoxyphenyl)amino]prop-1-yn-1-yl}-N-[(1S,4S)-4-{7- |
| | oxa-2-azaspiro[3.5]nonan-2-yl]cyclohexyl]-1-(2,2,2-trifluoroethyl)-1H-indol-4-amine |
| 994. | 2-{3-[(4-methanesulfonyl-2-methoxyphenyl)amino]prop-1-yn-1-yl}-N-[(1R,4R)-4-{7- |
| | oxa-2-azaspiro[3.5]nonan-2-yl]cyclohexyl]-1-(2,2,2-trifluoroethyl)-1H-indol-4-amine |
| 995. | 3-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 996. | 3-methoxy-N-methyl-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 997. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 998. | 3-methoxy-4-{[3-(4-{[(1S,4S)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 999. | 3-(cyanomethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1000. | 3-(cyanomethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1001. | 2-(3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1002. | 2-(4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 1003. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1004. | 2-(5-methanesulfonyl-2-{[3-(4-([(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1005. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1006. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1007. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1008. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1009. | methyl 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetate |
| 1010. | methyl 2-(2-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetate |
| 1011. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetic acid |
| 1012. | 2-(2-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-5-(methylsulfonyl)phenoxy)acetic acid |
| 1013. | 2-{3-[(4-methanesulfonyl-2-methoxyphenyl)amino]prop-1-yn-1-yl}-N-[(1R,4R)-4-{2- |
| | oxa-7-azaspiro[3.5]nonan-7-yl}cyclohexyl]-1-(2,2,2-trifluoroethyl)-1H-indol-4-amine |
| 1014. | 2-(3-{[4-(ethanesulfonyl)-2-methoxyphenyl]amino}prop-1-yn-1-yl)-N-[(1R,4R)-4-{7- |
| | oxa-2-azaspiro[3.5]nonan-2-yl}cyclohexyl]-1-(2,2,2-trifluoroethyl)-1H-indol-4-amine |
| 1015. | 2-(3-{[4-(ethanesulfonyl)-2-methoxyphenyl]amino}prop-1-yn-1-yl)-N-[(1S,4S)-4-{7-oxa- |
| | 2-azaspiro[3.5]nonan-2-yl}cyclohexyl]-1-(2,2,2-trifluoroethyl)-1H-indol-4-amine |
| 1016. | 3-(fluoromethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1017. | 3-(fluoromethoxy)-4-{[3-(4-{[(1S,4S)-4-{7-oxa-2-azaspiro[3,5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1018. | 3-methoxy-N-methyl-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1019. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 1020. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1021. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzenesulfonamide |
| 1022. | 2-{3-[(4-methanesulfonyl-2-methoxyphenyl)amino]prop-1-yn-1-yl}-N-[(1R,4R)-4-{6- |
| | oxa-2-azaspiro[3.5]nonan-2-yl}cyclohexyl]-1-(2,2,2-trifluoroethyl)-1H-indol-4-amine |
| 1023. | N-((1S,4S)-4-(6-oxa-2-azaspiro[3.5]nonan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1024. | N-((1R,4R)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1025. | N-((1S,4S)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1026. | 4-(3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)-7-(methylsulfonyl)-2H- |
| | benzo[b][1,4]oxazin-3(4H)-one |
| 1027. | 4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1028. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1029. | N-((1R,4R)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1030. | N-((1S,4S)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1031. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1032. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1033. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1034. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1035. | 2-fluoro-5-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1036. | 2-(3-((2-methoxy-4-(methylsulfonyl)phenyl)amino)prop-1-yn-1-yl)-N-((1R,4R)-4-(3- |
| | methoxypyrrolidin-1-yl)cyclohexyl)-1-(2,2,2-trifluoroethyl)-1H-indol-4-amine |
| 1037. | 2-(3-((2-methoxy-4-(methylsulfonyl)phenyl)amino)prop-1-yn-1-yl)-N-((1S,4S)-4-(3- |
| | methoxypyrrolidin-1-yl)cyclohexyl)-1-(2,2,2-trifluoroethyl)-1H-indol-4-amine |
| 1038. | 3-methoxy-4-((3-(4-(((1R,4R)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1039. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1040. | 2-(3-{[2-(fluoromethoxy)-4-methanesulfonylphenyl]amino}prop-1-yn-1-yl)-N-[(1R,4R)- |
| | 4-(morpholin-4-yl)cyclohexyl]-1-(2,2,2-trifluoroethyl)-1H-indol-4-amine |
| 1041. | 2-(3-{[2-(fluoromethoxy)-4-methanesulfonylphenyl]amino}prop-1-yn-1-yl)-N-[(1S,4S)- |
| | 4-(morpholin-4-yl)cyclohexyl]-1-(2,2,2-trifluoroethyl)-1H-indol-4-amine |
| 1042. | 3-(cyanomethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1043. | 3-(cyanomethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1044. | 3-(fluoromethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1045. | 3-(fluoromethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1046. | 2-(3-((2-ethoxy-4-(methylsulfonyl)phenyl)amino)prop-1-yn-1-yl)-N-((1R,4R)-4- |
| | morpholinocyclohexyl)-1-(2,2,2-trifluoroethyl)-1H-indol-4-amine |
| 1047. | 2-(3-((2-ethoxy-4-(methylsulfonyl)phenyl)amino)prop-1-yn-1-yl)-N-((1S,4S)-4- |
| | morpholinocyclohexyl)-1-(2,2,2-trifluoroethyl)-1H-indol-4-amine |
| 1048. | 2-{3-[(4-methanesulfonyl-2-methoxyphenyl)amino]prop-1-yn-1-yl}-N-[(1R,4R)-4-(3- |
| | methoxypiperidin-1-yl)cyclohexyl]-1-(2,2,2-trifluoroethyl)-1H-indol-4-amine |
| 1049. | 2-(3-((2-methoxy-4-(methylsulfonyl)phenyl)amino)prop-1-yn-1-yl)-N-((1S,4S)-4-(3- |
| | methoxypiperidin-1-yl)cyclohexyl)-1-(2,2,2-trifluoroethyl)-1H-indol-4-amine |
| 1050. | 2-{3-[(4-methanesulfonyl-2-methoxyphenyl)amino]prop-1-yn-1-yl}-N-[(1R,4R)-4-(4- |
| | methoxypiperidin-1-yl)cyclohexyl]-1-(2,2,2-trifluoroethyl)-1H-indol-4-amine |
| 1051. | 2-(3-((2-methoxy-4-(methylsulfonyl)phenyl)amino)prop-1-yn-1-yl)-N-((1S,4S)-4-(4- |
| | methoxypiperidin-1-yl)cyclohexyl)-1-(2,2,2-trifluoroethyl)-1H-indol-4-amine |
| 1052. | 3-methoxy-N-methyl-4-{[3-(4-{[(1R,4R)-4-(4-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1053. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1054. | 3-methoxy-4-{[3-(4-{[(1R,4R)-4-(4-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1055. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1056. | 3-methoxy-N-methyl-4-{[3-(4-{[(1R,4R)-4-(3-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1057. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1058. | 3-methoxy-4-{[3-(4-{[(1R,4R)-4-(3-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1059. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1060. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 1061. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 1062. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁶-thiomorpholine-1,1-dione |
| 1063. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1,1-dioxide |
| 1064. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1065. | 4-[(1S,4S)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1066. | 4-((3-(4-(((1R,4R)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1067. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1068. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1-oxide |
| 1069. | 3-methoxy-N-methyl-4-{[3-(4-{[(1R,4R)-4-(1-oxo-1λ⁴-thiomorpholin-4- |
| | yl)cyclohexyl]amino}-1-(2,2,2-trifluoroethyl)-1H-indol-2-yl)prop-2-yn-1- |
| | yl]amino}benzamide |
| 1070. | 3-methoxy-4-{[3-(4-{[(1R,4R)-4-(1-oxo-1λ⁴-thiomorpholin-4-yl)cyclohexyl]amino}-1- |
| | (2,2,2-trifluoroethyl)-1H-indol-2-yl)prop-2-yn-1-yl]amino}benzene-1-sulfonamide |
| 1071. | 3-methoxy-4-((3-(4-(((1S,4S)-4-(1-oxidothiomorpholino)cyclohexyl)amino)-1-(2,2,2- |
| | trifluoroethyl)-1H-indol-2-yl)prop-2-yn-1-yl)amino)benzenesulfonamide |
| 1072. | 3-methoxy-4-{[3-(4-{[(1R,4R)-4-(1,1-dioxo-1λ⁶-thiomorpholin-4-yl)cyclohexyl]amino}- |
| | 1-(2,2,2-trifluoroethyl)-1H-indol-2-yl)prop-2-yn-1-yl]amino}benzene-1-sulfonamide |
| 1073. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxybenzenesulfonamide |
| 1074. | 2-{3-[(4-methanesulfonyl-2-methoxyphenyl)amino]prop-1-yn-1-yl}-N-[(1S,4S)-4-{2- |
| | oxa-7-azaspiro[3.5]nonan-7-yl}cyclohexyl]-1-(2,2,2-trifluoroethyl)-1H-indol-4-amine |
| 1075. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1076. | 3-methoxy-N-methyl-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1077. | 3-methoxy-4-{[3-(4-{[(1R,4R)-4-{2-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1078. | 4-((3-(4-(((1S,4S)-4-(2-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1079. | 3-(cyanomethoxy)-4-][3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1080. | 3-(cyanomethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1081. | 3-(fluoromethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1082. | 3-(fluoromethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1083. | 2-{3-[(4-methanesulfonyl-2-methoxyphenyl)amino]prop-1-yn-1-yl}-N-[(1R,4R)-4-{1- |
| | oxa-7-azaspiro[3.5]nonan-7-yl}cyclohexyl]-1-(2,2,2-trifluoroethyl)-1H-indol-4-amine |
| 1084. | N-((1S,4S)-4-(1-oxa-7-azaspiro[3.5]nonan-7-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1085. | 3-methoxy-N-methyl-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1086. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxy-N-methylbenzamide |
| 1087. | 3-methoxy-4-{[3-(4-{[(1R,4R)-4-{1-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1088. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1089. | 2-(5-methanesulfonyl-2-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1090. | 2-(5-methanesulfonyl-2-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1091. | 2-(3-{[2-(fluoromethoxy)-4-methanesulfonylphenyl]amino}prop-1-yn-1-yl)-N-[(1R,4R)- |
| | 4-{2-oxa-7-azaspiro[3.5]nonan-7-yl}cyclohexyl]-1-(2,2,2-trifluoroethyl)-1H-indol-4- |
| | amine |
| 1092. | 2-(3-{[2-(fluoromethoxy)-4-methanesulfonylphenyl]amino}prop-1-yn-1-yl)-N-[(1S,4S)- |
| | 4-{2-oxa-7-azaspiro[3.5]nonan-7-yl}cyclohexyl]-1-(2,2,2-trifluoroethyl)-1H-indol-4- |
| | amine |
| 1093. | N-((1R,4R)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1094. | N-((1S,4S)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1095. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1096. | N-((1R,4R)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1097. | N-((1S,4S)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1098. | 2-{3-[(4-methanesulfonyl-2-methoxyphenyl)amino]prop-1-yn-1-yl}-N-[(1R,4R)-4-{6- |
| | oxa-3-azabicyclo[3.1.1]heptan-3-yl}cyclohexyl]-1-(2,2,2-trifluoroethyl)-1H-indol-4- |
| | amine |
| 1099. | 2-{3-[(4-methanesulfonyl-2-methoxyphenyl)amino]prop-1-yn-1-yl}-N-[(1S,4S)-4-{6- |
| | oxa-3-azabicyclo[3.1.1]heptan-3-yl}cyclohexyl]-1-(2,2,2-trifluoroethyl)-1H-indol-4- |
| | amine |
| 1100. | 3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1101. | 3-methoxy-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1102. | 3-methoxy-N-methyl-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1103. | 3-methoxy-N-methyl-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1104. | 2-{3-[(2-ethoxy-4-methanesulfonylphenyl)amino]prop-1-yn-1-yl}-N-[(1R,4R)-4-{6-oxa- |
| | 3-azabicyclo[3.1.1]heptan-3-yl}cyclohexyl]-1-(2,2,2-trifluoroethyl)-1H-indol-4-amine |
| 1105. | 2-{3-[(2-ethoxy-4-methanesulfonylphenyl)amino]prop-1-yn-1-yl}-N-[(1S,4S)-4-{6-oxa- |
| | 3-azabicyclo[3.1.1]heptan-3-yl}cyclohexyl]-1-(2,2,2-trifluoroethyl)-1H-indol-4-amine |
| 1106. | N-ethyl-3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1107. | 2-(5-methanesulfonyl-2-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}phenoxy)acetonitrile |
| 1108. | 2-(2-((3-(4-(((1S,4S)-4-(6-oxa-3-azabicyclo[3.1.1]heptan-3-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetonitrile |
| 1109. | 3-methoxy-N-methyl-4-{[3-(4-{[(1R,4R)-4-[2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzamide |
| 1110. | 2-{3-[(4-methanesulfonyl-2-methoxyphenyl)amino]prop-1-yn-1-yl}-N-[(1R,4R)-4-{2- |
| | oxa-8-azaspiro[4.5]decan-8-yl}cyclohexyl]-1-(2,2,2-trifluoroethyl)-1H-indol-4-amine |
| 1111. | 3-methoxy-4-{[3-(4-{[(1R,4R)-4-{2-oxa-8-azaspiro[4.5]decan-8-yl}cyclohexyl]amino}- |
| | 1-(2,2,2-trifluoroethyl)-1H-indol-2-yl)prop-2-yn-1-yl]amino}benzene-1-sulfonamide |
| 1112. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1113. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1114. | 2-{3-[(4-methanesulfonyl-2-methoxyphenyl)amino]prop-1-yn-1-yl}-N-[(1R,4R)-4-{3- |
| | oxa-9-azaspiro[5.5]undecan-9-yl}cyclohexyl]-1-(2,2,2-trifluoroethyl)-1H-indol-4-amine |
| 1115. | N-((1S,4S)-4-(3-oxa-9-azaspiro[5.5]undecan-9-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1116. | 3-methoxy-N-methyl-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzamide |
| 1117. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxy-N-methylbenzamide |
| 1118. | 3-methoxy-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzene-1-sulfonamide |
| 1119. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxybenzenesulfonamide |
| 1120. | 3-methoxy-N-methyl-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzamide |
| 1121. | 3-methoxy-N-methyl-4-((3-(4-(((1S,4S)-4-(pyrrolidin-1-yl)cyclohexyl)amino)-1-(2,2,2- |
| | trifluoroethyl)-1H-indol-2-yl)prop-2-yn-1-yl)amino)benzamide |
| 1122. | 3-methoxy-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1123. | 3-methoxy-4-((3-(4-(((1S,4S)-4-(pyrrolidin-1-yl)cyclohexyl)amino)-1-(2,2,2- |
| | trifluoroethyl)-1H-indol-2-yl)prop-2-yn-1-yl)amino)benzenesulfonamide |
| 1124. | 2-{3-[(4-methanesulfonyl-2-methoxyphenyl)amino]prop-1-yn-1-yl}-N-[(1R,4R)-4- |
| | (pyrrolidin-1-yl)cyclohexyl]-1-(2,2,2-trifluoroethyl)-1H-indol-4-amine |
| 1125. | 2-(3-((2-methoxy-4-(methylsulfonyl)phenyl)amino)prop-1-yn-1-yl)-N-((1S,4S)-4- |
| | (pyrrolidin-1-yl)cyclohexyl)-1-(2,2,2-trifluoroethyl)-1H-indol-4-amine |
| 1126. | -{3-[(4-methanesulfonyl-2-methoxyphenyl)amino]prop-1-yn-1-yl}-N-[(1R,4R)-4-(3- |
| | methoxypyrrolidin-1-yl)cyclohexyl]-1-(2,2,2-trifluoroethyl)-1H-indol-4-amine |
| 1127. | 3-methoxy-4-{[3-(4-{[(1R,4R)-4-(3-methoxypyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1128. | 3-methoxy-N-methyl-4-{[3-(4-{[(1R,4R)-4-(3-methoxypyrrolidin-1- |
| | yl)cyclohexyl]amino}-1-(2,2,2-trifluoroethyl)-1H-indol-2-yl)prop-2-yn-1- |
| | yl]amino}benzamide |
| 1129. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)-N-methylbenzamide |
| 1130. | 3-methoxy-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzene-1-sulfonamide |
| 1131. | 3-methoxy-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzenesulfonamide |
| 1132. | 2-{3-[(4-methanesulfonyl-2-methoxyphenyl)amino]prop-1-yn-1-yl}-N-[(1R,4R)-4- |
| | {hexahydro-1H-furo[3,4-c]pyrrol-5-yl}cyclohexyl]-1-(2,2,2-trifluoroethyl)-1H-indol-4- |
| | amine |
| 1133. | 2-(3-((2-methoxy-4-(methylsulfonyl)phenyl)amino)prop-1-yn-1-yl)-N-((1R,4R)-4- |
| | (tetrahydro-1H-furo[3,4-c]pyrrol-5(3H)-yl)cyclohexyl)-1-(2,2,2-trifluoroethyl)-1H-indol- |
| | 4-amine |
| 1134. | 3-methoxy-N-methyl-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzamide |
| 1135. | 3-methoxy-N-methyl-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzamide |
| 1136. | 2-{3-[(4-methanesulfonyl-2-methoxyphenyl)amino]prop-1-yn-1-yl}-N-[(1R,4R)-4-[4- |
| | (trifluoromethyl)piperidin-1-yl]cyclohexyl]-1-(2,2,2-trifluoroethyl)-1H-indol-4-amine |
| 1137. | 2-(3-((2-methoxy-4-(methylsulfonyl)phenyl)amino)prop-1-yn-1-yl)-1-(2,2,2- |
| | trifluoroethyl)-N-((1S,4S)-4-(4-(trifluoromethyl)piperidin-1-yl)cyclohexyl)-1H-indol-4- |
| | amine |
| 1138. | 3-methoxy-N-methyl-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzamide |
| 1139. | 3-methoxy-N-methyl-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzamide |
| 1140. | 3-methoxy-4-{[3-(4-{[(1R,4R)-4-[4-(trifluoromethyl)piperidin-1-yl]cyclohexyl]amino}- |
| | 1-(2,2,2-trifluoroethyl)-1H-indol-2-yl)prop-2-yn-1-yl]amino}benzene-1-sulfonamide |
| 1141. | 3-methoxy-4-((3-(1-(2,2,2-trifluoroethyl)-4-(((1S,4S)-4-(4-(trifluoromethyl)piperidin-1- |
| | yl)cyclohexyl)amino)-1H-indol-2-yl)prop-2-yn-1-yl)amino)benzenesulfonamide |
| 1142. | 2-{3-[(4-methanesulfonyl-2-methoxyphenyl)amino]prop-1-yn-1-yl}-N-[(1R,4R)-4-(4- |
| | methanesulfonylpiperidin-1-yl)cyclohexyl]-1-(2,2,2-trifluoroethyl)-1H-indol-4-amine |
| 1143. | 2-(3-((2-methoxy-4-(methylsulfonyl)phenyl)amino)prop-1-yn-1-yl)-N-((1S,4S)-4-(4- |
| | (methylsulfonyl)piperidin-1-yl)cyclohexyl)-1-(2,2,2-trifluoroethyl)-1H-indol-4-amine |
| 1144. | 3-methoxy-N-methyl-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzamide |
| 1145. | 3-methoxy-N-methyl-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzamide |
| 1146. | 3-methoxy-4-{[3-(4-{[(1R,4R)-4-(4-methanesulfonylpiperidin-1-yl)cyclohexyl]amino}-1- |
| | (2,2,2-trifluoroethyl)-1H-indol-2-yl)prop-2-yn-1-yl]amino}benzene-1-sulfonamide |
| 1147. | 3-methoxy-4-((3-(4-(((1S,4S)-4-(4-(methylsulfonyl)piperidin-1-yl)cyclohexyl)amino)-1- |
| | (2,2,2-trifluoroethyl)-1H-indol-2-yl)prop-2-yn-1-yl)amino)benzenesulfonamide |
| 1148. | 1-[(1S,3R)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-4,5-dihydro-1H-1,2,3,4-tetrazol- |
| | 5-one |
| 1149. | 2-(3-((2-methoxy-4-(methylsulfonyl)phenyl)amino)prop-1-yn-1-yl)-N-((1R,4R)-4- |
| | morpholinocyclohexyl)-1-(oxiran-2-ylmethyl)-1H-indol-4-amine |
| 1150. | 2-((2-(3-((2-methoxy-4-(methylsulfonyl)phenyl)amino)prop-1-yn-1-yl)-4-(((1R,4R)-4- |
| | morpholinocyclohexyl)amino)-1H-indol-1-yl)methyl)acrylonitrile |
| 1151. | N-((1R,4R)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1152. | N-((1S,4S)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1153. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1154. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1155. | 1-[1-(2-hydroxyethyl)piperidin-4-yl]-3-(2-{3-[(4-methanesulfonyl-2- |
| | methoxyphenyl)amino]prop-1-yn-1-yl}-1-(2,2,2-trifluoroethyl)-1H-indol-4-yl)urea |
| 1156. | 3-methoxy-4-({3-[4-({2-oxaspiro[3.3]heptan-6-yl}amino)-1-(2,2,2-trifluoroethyl)-1H- |
| | indol-2-yl]prop-2-yn-1-yl}amino)benzene-1-sulfonamide |
| 1157. | [1-(chloromethyl)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclobutyl]methanol |
| 1158. | 2-{3-[(4-methanesulfonyl-2-methoxyphenyl)amino]prop-1-yn-1-yl}-N-{2- |
| | oxaspiro[3.3]heptan-6-yl}-1-(2,2,2-trifluoroethyl)-1H-indol-4-amine |
| 1159. | 4-({3-[4-({2-azaspiro[3.3]heptan-6-yl}amino)-1-(2,2,2-trifluoroethyl)-1H-indol-2- |
| | yl]prop-2-yn-1-yl}amino)-3-methoxybenzene-1-sulfonamide |
| 1160. | N-{2-azaspiro[3.3]heptan-6-yl}-2-{3-[(4-methanesulfonyl-2-methoxyphenyl)amino]prop- |
| | 1-yn-1-yl}-1-(2,2,2-trifluoroethyl)-1H-indol-4-amine |
| 1161. | 3-methoxy-N-methyl-4-{[3-(4-{[(1R,4R)-4-(methylamino)cyclohexyl]amino}-1-(2,2,2- |
| | trifluoroethyl)-1H-indol-2-yl)prop-2-yn-1-yl]amino}benzamide |
| 1162. | 3-methoxy-N-methyl-4-{[3-(4-{[(1R,4R)-4-aminocyclohexyl]amino}-1-(2,2,2- |
| | trifluoroethyl)-1H-indol-2-yl)prop-2-yn-1-yl]amino}benzamide |
| 1163. | 3-methoxy-N-methyl-4-{[3-(4-{[(1S,4S)-4-aminocyclohexyl]amino}-1-(2,2,2- |
| | trifluoroethyl)-1H-indol-2-yl)prop-2-yn-1-yl]amino}benzamide |
| 1164. | re1-(1R,3R)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 1165. | rac-(1R,3S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 1166. | (1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamine |
| 1167. | rac-(1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamine |
| 1168. | rel-(1R,3S)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexan-1-ol |
| 1169. | 3-methoxy-N-methyl-4-{[3-(4-{[(1R,4R)-4-hydroxy-4-methylcyclohexyl]amino}-1- |
| | (2,2,2-trifluoroethyl)-1H-indol-2-yl)prop-2-yn-1-yl]amino}benzamide |
| 1170. | 3-methoxy-N-methyl-4-{[3-(4-{[(1S,4S)-4-hydroxy-4-methylcyclohexyl]amino}-1- |
| | (2,2,2-trifluoroethyl)-1H-indol-2-yl)prop-2-yn-1-yl]amino}benzamide |
| 1171. | 4-[(3-{4-[(4-cyanocyclohexyl)amino]-1-(2,2,2-trifluoroethyl)-1H-indol-2-yl}prop-2-yn-1- |
| | yl)amino]-3-methoxy-N-methylbenzamide |
| 1172. | 3-methoxy-N-methyl-4-{[3-(4-{[(1R,4R)-4-cyanocyclohexyl]amino}-1-(2,2,2- |
| | trifluoroethyl)-1H-indol-2-yl)prop-2-yn-1-yl]amino}benzamide |
| 1173. | 3-methoxy-N-methyl-4-{[3-(4-{[(1S,4S)-4-cyanocyclohexyl]amino}-1-(2,2,2- |
| | trifluoroethyl)-1H-indol-2-yl)prop-2-yn-1-yl]amino}benzamide |
| 1174. | 3-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexane-1-carboxylic acid |
| 1175. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)cyclohexane-1,4-diamine |
| 1176. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)cyclohexane-1,4-diamine |
| 1177. | 2-fluoro-N¹-(2-{3-[(4-methanesulfonyl-2-methoxyphenyl)amino]prop-1-yn-1-yl}-1- |
| | (2,2,2-trifluoroethyl)-1H-indol-4-yl)-N⁴-methylcyclohexane-1,4-diamine |
| 1178. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴-methylcyclohexane-1,4-diamine |
| 1179. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 1180. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴,N⁴-dimethylcyclohexane-1,4-diamine |
| 1181. | (3S,4R)-3-fluoro-4-[(2-{3-[(4-methanesulfonyl-2-methoxyphenyl)amino]prop-1-yn-1-yl}- |
| | 1-(2,2,2-trifluoroethyl)-1H-indol-4-yl)amino]cyclohexan-1-ol |
| 1182. | 3-methoxy-4-{[3-(4-{[(1R,4R)-4-[bis(2-hydroxyethyl)amino]cyclohexyl]amino}-1- |
| | (2,2,2-trifluoroethyl)-1H-indol-2-yl)prop-2-yn-1-yl]amino}benzene-1-sulfonamide |
| 1183. | 3-methoxy-4-{[3-(4-{[(1S,4S)-4-[bis(2-hydroxyethyl)amino]cyclohexyl]amino}-1-(2,2,2- |
| | trifluoroethyl)-1H-indol-2-yl)prop-2-yn-1-yl]amino}benzene-1-sulfonamide |
| 1184. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 1185. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 1186. | 3-methoxy-N-(1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1187. | 3-methoxy-N-(oxan-4-yl)-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1188. | 3-methoxy-N-(1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1189. | 3-methoxy-N-(oxan-4-yl)-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1190. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 1191. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 1192. | 3-methoxy-N-(5-methyl-1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1193. | 3-methoxy-N-(5-methyl-1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1194. | (1S,4S)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 1195. | (1R,4R)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 1196. | 3-methoxy-4-{[3-(4-{[(1S,4S)-4-[(3S,4S)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 1197. | 4-((3-(4-(((1R,4R)-4-((3R,4R)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 1198. | 4-((3-(4-(((1R,4S)-4-((3S,4S)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 1199. | 3-methoxy-4-{[3-(4-{[(1R,4R)-4-[bis(2-methoxyethyl)amino]cyclohexyl]amino}-1- |
| | (2,2,2-trifluoroethyl)-1H-indol-2-yl)prop-2-yn-1-yl]amino}benzene-1-sulfonamide |
| 1200. | 3-methoxy-4-{[3-(4-{[(1S,4S)-4-[bis(2-methoxyethyl)amino]cyclohexyl]amino}-1-(2,2,2- |
| | trifluoroethyl)-1H-indol-2-yl)prop-2-yn-1-yl]amino}benzene-1-sulfonamide |
| 1201. | 4-({3-[4-({1,4-dioxaspiro[4.5]decan-8-yl}amino)-1-(2,2,2-trifluoroethyl)-1H-indol-2- |
| | yl]prop-2-yn-1-yl}amino)-3-methoxybenzene-1-sulfonamide |
| 1202. | N-{1,4-dioxaspiro[4.5]decan-8-yl}-2-{3-[(4-methanesulfonyl-2- |
| | methoxyphenyl)amino]prop-1-yn-1-yl}-1-(2,2,2-trifluoroethyl)-1H-indol-4-amine |
| 1203. | 3-methoxy-4-[(3-{4-[(4-oxocyclohexyl)amino]-1-(2,2,2-trifluoroethyl)-1H-indol-2- |
| | yl}prop-2-yn-1-yl)amino]benzene-1-sulfonamide |
| 1204. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-one |
| 1205. | (1R,4R)-N⁴-[2-(3-{[2-methoxy-4-(morpholine-4-sulfonyl)phenyl]amino}prop-1-yn-1- |
| | yl)-1-(2,2,2-trifluoroethyl)-1H-indol-4-yl]-N¹,N¹-dimethylcyclohexane-1,4-diamine |
| 1206. | (1S,4S)-N⁴-[2-(3-{[2-methoxy-4-(morpholine-4-sulfonyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 1207. | N,N-bis(2-hydroxyethyl)-3-methoxy-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1208. | N,N-bis(2-hydroxyethyl)-3-methoxy-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1209. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 1210. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 1211. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1212. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1213. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1214. | (1R,4R)-N⁴-{2-[3-({2-methoxy-4-[(4-methylpiperazin-1-yl)sulfonyl]phenyl}amino)prop- |
| | 1-yn-1-yl]-1-(2,2,2-trifluoroethyl)-1H-indol-4-yl}-N¹,N¹-dimethylcyclohexane-1,4- |
| | diamine |
| 1215. | (1R,4R)-N⁴-(2-{3-[(4-methanesulfonyl-2-methoxyphenyl)amino]prop-1-yn-1-yl}-1- |
| | [(oxiran-2-yl)methyl]-1H-indol-4-yl)-N¹,N¹-dimethylcyclohexane-1,4-diamine |
| 1216. | 2-[(2-{3-[(4-methanesulfonyl-2-methoxyphenyl)amino]prop-1-yn-1-yl}-4-{[(1R,4R)-4- |
| | (dimethylamino)cyclohexyl]amino}-1H-indol-1-yl)methyl]prop-2-enenitrile |
| 1217. | 5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenol |
| 1218. | 5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenol |
| 1219. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 1220. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 1221. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 1222. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 1223. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 1224. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 1225. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 1226. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 1227. | (1R,4R)-N⁴-[2-(3-{[4-methanesulfonyl-2-(2-methoxyethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 1228. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 1229. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 1230. | (1R,4R)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 1231. | (1S,4S)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 1232. | (1S,4S)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 1233. | (1R,4R)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 1234. | (1S,4S)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 1235. | 3-hydroxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1236. | 3-hydroxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1237. | 3-(2-methoxyethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzamide |
| 1238. | (1R,4R)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 1239. | N-ethyl-3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1240. | N-ethyl-3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1241. | 3-(2-methoxyethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzamide |
| 1242. | 3-(2-fluoroethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1243. | 3-(2-fluoroethoxy)-N-methyl-4-{[3-(4-{[(1S,4S)-4-(dimethylamino)cyclohexyl]amino}- |
| | 1-(2,2,2-trifluoroethyl)-1H-indol-2-yl)prop-2-yn-1-yl]amino}benzamide |
| 1244. | 3-(fluoromethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1245. | 3-(fluoromethoxy)-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 1246. | (1S,4S)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 1247. | (1R,4R)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 1248. | 3-methoxy-N-methyl-4-{[3-(4-{[(1S,4S)-4-(methylamino)cyclohexyl]amino}-1-(2,2,2- |
| | trifluoroethyl)-1H-indol-2-yl)prop-2-yn-1-yl]amino}benzamide |
| 1249. | (1S,4S)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 1250. | (1R,4R)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 1251. | 3-(cyanomethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1252. | 3-(2-cyanoethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1253. | N-(5-methanesulfonyl-2-{[3-(4-{[(1R,4R)-4-(dimethylamino)cyclohexyl]amino}-1- |
| | (2,2,2-trifluoroethyl)-1H-indol-2-yl)prop-2-yn-1-yl]oxy}phenyl)acetamide |
| 1254. | N-(2-((3-(4-(((1S,4S)-4-(dimethylamino)cyclohexyl)amino)-1-(2,2,2-trifluoroethyl)-1H- |
| | indol-2-yl)prop-2-yn-1-yl)oxy)-5-(methylsulfonyl)phenyl)acetamide |
| 1255. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 1256. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 1257. | (1R,4R)-N⁴-[2-(3-{[4-(ethanesulfonyl)-2-methoxyphenyl]amino}prop-1-yn-1-yl)-1- |
| | (2,2,2-trifluoroethyl)-1H-indol-4-yl]-N¹,N¹-dimethylcyclohexane-1,4-diamine |
| 1258. | (1S,4S)-N⁴-[2-(3-{[4-(ethanesulfonyl)-2-methoxyphenyl]amino}prop-1-yn-1-yl)-1-(2,2,2- |
| | trifluoroethyl)-1H-indol-4-yl]-N¹,N¹-dimethylcyclohexane-1,4-diamine |
| 1259. | 2-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenyl)-2-methylpropanenitrile |
| 1260. | 2-(4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxyphenyl)-2-methylpropanenitrile |
| 1261. | 3-(cyanomethoxy)-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 1262. | 3-(cyanomethoxy)-4-((3-(4-(((1S,4S)-4-(dimethylamino)cyclohexyl)amino)-1-(2,2,2- |
| | trifluoroethyl)-1H-indol-2-yl)prop-2-yn-1-yl)amino)benzenesulfonamide |
| 1263. | 3-(3-(4-(((1R,4R)-4-(dimethylamino)cyclohexyl)amino)-1-(2,2,2-trifluoroethyl)-1H- |
| | indol-2-yl)prop-2-yn-1-yl)-6-(methylsulfonyl)benzo[d]oxazol-2(3H)-one |
| 1264. | 3-(3-(4-(((1S,4S)-4-(dimethylamino)cyclohexyl)amino)-1-(2,2,2-trifluoroethyl)-1H-indol- |
| | 2-yl)prop-2-yn-1-yl)-6-(methylsulfonyl)benzo[d]oxazol-2(3H)-one |
| 1265. | 3-methoxy-N,N-dimethyl-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1266. | (1R,4R)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 1267. | (1S,4S)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 1268. | (1R,4R)-N⁴-(2-{3-[(4-chloro-2-methoxyphenyl)amino]prop-1-yn-1-yl}-1-(2,2,2- |
| | trifluoroethyl)-1H-indol-4-yl)-N¹,N¹-dimethylcyclohexane-1,4-diamine |
| 1269. | (1S,4S)-N¹-(2-(3-((4-chloro-2-methoxyphenyl)amino)prop-1-yn-1-yl)-1-(2,2,2- |
| | trifluoroethyl)-1H-indol-4-yl)-N⁴,N⁴-dimethylcyclohexane-1,4-diamine |
| 1270. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 1271. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 1272. | (1R,4R)-N⁴-[2-(3-{[2-methoxy-4-(trifluoromethyl)phenyl]amino}prop-1-yn-1-yl)-1- |
| | (2,2,2-trifluoroethyl)-1H-indol-4-yl]-N¹,N¹-dimethylcyclohexane-1,4-diamine |
| 1273. | (1S,4S)-N¹-(2-(3-((2-methoxy-4-(trifluoromethyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 1274. | 2-fluoro-5-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1275. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxy-N-methylbenzamide |
| 1276. | (1S,4S)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 1277. | (1R,4R)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 1278. | (1R,4R)-N⁴-(2-{3-[(4-methanesulfonyl-2-methylphenyl)amino]prop-1-yn-1-yl}-1-(2,2,2- |
| | trifluoroethyl)-1H-indol-4-yl)-N¹,N¹-dimethylcyclohexane-1,4-diamine |
| 1279. | (1S,4S)-N¹,N¹-dimethyl-N⁴-(2-(3-((2-methyl-4-(methylsulfonyl)phenyl)amino)prop-1-yn- |
| | 1-yl)-1-(2,2,2-trifluoroethyl)-1H-indol-4-yl)cyclohexane-1,4-diamine |
| 1280. | (1R,4R)-N⁴-[2-(3-{[4-methanesulfonyl-2-(trifluoromethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 1281. | (1S,4S)-N¹,N¹-dimethyl-N⁴-(2-(3-((4-(methylsulfonyl)-2- |
| | (trifluoromethoxy)phenyl)amino)prop-1-yn-1-yl)-1-(2,2,2-trifluoroethyl)-1H-indol-4- |
| | yl)cyclohexane-1,4-diamine |
| 1282. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzamide |
| 1283. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxy-N-methylbenzamide |
| 1284. | 3-methoxy-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 1285. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxybenzenesulfonamide |
| 1286. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 1287. | 2-(5-methanesulfonyl-2-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 1288. | 2-(5-methanesulfonyl-2-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 1289. | 3-methoxy-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1290. | 3-methoxy-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1291. | N-ethyl-3-methoxy-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1292. | N-ethyl-3-methoxy-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)benzamide |
| 1293. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 1294. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 1295. | 3-(fluoromethoxy)-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1296. | 3-(fluoromethoxy)-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1297. | 3-(fluoromethoxy)-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)-N-methylbenzamide |
| 1298. | 3-methoxy-4-{[3-(4-{[(1R,4R)-4-[(2-methoxyethyl)(methyl)amino]cyclohexyl]amino}-1- |
| | (2,2,2-trifluoroethyl)-1H-indol-2-yl)prop-2-yn-1-yl]amino}benzene-1-sulfonamide |
| 1299. | 3-methoxy-4-((3-(4-(((1S,4S)-4-((2-methoxyethyl)(methyl)amino)cyclohexyl)amino)-1- |
| | (2,2,2-trifluoroethyl)-1H-indol-2-yl)prop-2-yn-1-yl)amino)benzenesulfonamide |
| 1300. | (1R,4R)-N¹,N¹-diethyl-N⁴-(2-{3-[(4-methanesulfonyl-2-methoxyphenyl)amino]prop-1- |
| | yn-1-yl}-1-(2,2,2-trifluoroethyl)-1H-indol-4-yl)cyclohexane-1,4-diamine |
| 1301. | (1S,4S)-N¹,N¹-diethyl-N⁴-(2-(3-((2-methoxy-4-(methylsulfonyl)phenyl)amino)prop-1-yn- |
| | 1-yl)-1-(2,2,2-trifluoroethyl)-1H-indol-4-yl)cyclohexane-1,4-diamine |
| 1302. | 3-(cyanomethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1303. | 3-(cyanomethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1304. | 3-(fluoromethoxy)-4-{[3-(4-{[(1R,4R)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 1305. | 3-(fluoromethoxy)-4-{[3-(4-{[(1S,4S)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 1306. | 3-(fluoromethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1307. | 3-(fluoromethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1308. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 1309. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 1310. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 1311. | 3-methoxy-N-methyl-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 1312. | 2-(2-{3-[(4-methanesulfonyl-2-methoxyphenyl)amino]prop-1-yn-1-yl}-4-{[(1R,4R)-4- |
| | (morpholin-4-yl)cyclohexyl]amino}-1H-indol-1-yl)acetonitrile |
| 1313. | 4-({3-[1-(2-fluoroethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 1314. | 1-(2-fluoroethyl)-2-{3-[(4-methanesulfonyl-2-methoxyphenyl)amino]prop-1-yn-1-yl}-N- |
| | [(1R,4R)-4-(morpholin-4-yl)cyclohexyl]-1H-indol-4-amine |
| 1315. | 4-({3-[1-(cyanomethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 1316. | 2-{3-[(4-methanesulfonyl-2-methoxyphenyl)amino]prop-1-yn-1-yl}-1-propyl-N- |
| | [(1R,4R)-4-(morpholin-4-yl)cyclohexyl]-1H-indol-4-amine |
| 1317. | 2-{3-[(4-methanesulfonyl-2-methoxyphenyl)amino]prop-1-yn-1-yl}-1-(2-methylpropyl)- |
| | N-[(1R,4R)-4-(morpholin-4-yl)cyclohexyl]-1H-indol-4-amine |
| 1318. | 1-(2,2-difluoroethyl)-2-{3-[(4-methanesulfonyl-2-methoxyphenyl)amino]prop-1-yn-1- |
| | yl}-N-[(1R,4R)-4-(morpholin-4-yl)cyclohexyl]-1H-indol-4-amine |
| 1319. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 1320. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 1321. | N-ethyl-3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 1322. | N-ethyl-3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzamide |
| 1323. | 2-(5-methanesulfonyl-2-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 1324. | 2-(5-methanesulfonyl-2-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 1325. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1326. | 2-{3-[(4-methanesulfonyl-2-methoxyphenyl)amino]prop-1-yn-1-yl}-N-[(1R,4R)-4-(3- |
| | methanesulfonylazetidin-1-yl)cyclohexyl]-1-(2,2,2-trifluoroethyl)-1H-indol-4-amine |
| 1327. | 2-(3-((2-methoxy-4-(methylsulfonyl)phenyl)amino)prop-1-yn-1-yl)-N-((1S,4S)-4-(3- |
| | (methylsulfonyl)azetidin-1-yl)cyclohexyl)-1-(2,2,2-trifluoroethyl)-1H-indol-4-amine |
| 1328. | 3-methoxy-4-{[3-(4-{[(1R,4R)-4-(3-methanesulfonylazetidin-1-yl)cyclohexyl]amino}-1- |
| | (2,2,2-trifluoroethyl)-1H-indol-2-yl)prop-2-yn-1-yl]amino}benzene-1-sulfonamide |
| 1329. | 3-methoxy-4-((3-(4-(((1S,4S)-4-(3-(methylsulfonyl)azetidin-1-yl)cyclohexyl)amino)-1- |
| | (2,2,2-trifluoroethyl)-1H-indol-2-yl)prop-2-yn-1-yl)amino)benzenesulfonamide |
| 1330. | 3-methoxy-N-methyl-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzamide |
| 1331. | 3-methoxy-N-methyl-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzamide |
| 1332. | 2-{3-[(4-methanesulfonyl-2-methoxyphenyl)amino]prop-1-yn-1-yl}-N-[(1R,4R)-4-{2- |
| | azaspiro[3.3]heptan-2-yl}cyclohexyl]-1-(2,2,2-trifluoroethyl)-1H-indol-4-amine |
| 1333. | N-((1S,4S)-4-(2-azaspiro[3.3]heptan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1334. | 3-methoxy-N-methyl-4-{[3-(4-{[(1R,4R)-4-{2-azaspiro[3.3]heptan-2- |
| | yl}cyclohexyl]amino}-1-(2,2,2-trifluoroethyl)-1H-indol-2-yl)prop-2-yn-1- |
| | yl]amino}benzamide |
| 1335. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 1336. | 3-methoxy-4-{[3-(4-{[(1R,4R)-4-{2-azaspiro[3.3]heptan-2-yl}cyclohexyl]amino}-1- |
| | (2,2,2-trifluoroethyl)-1H-indol-2-yl)prop-2-yn-1-yl]amino}benzene-1-sulfonamide |
| 1337. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxybenzenesulfonamide |
| 1338. | 2-(3-((2-methoxy-4-(methylsulfonyl)phenyl)amino)prop-1-yn-1-yl)-N-(2- |
| | azaspiro[3.3]heptan-6-yl)-1-(2,2,2-trifluoroethyl)-1H-indol-4-amine |
| 1339. | 4-((3-(4-((2-azaspiro[3.3]heptan-6-yl)amino)-1-(2,2,2-trifluoroethyl)-1H-indol-2-yl)prop- |
| | 2-yn-1-yl)amino)-3-methoxybenzenesulfonamide |
| 1340. | 2-{3-[(4-methanesulfonyl-2-methoxyphenyl)amino]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 1341. | 2-{3-[(4-methanesulfonyl-2-methoxyphenyl)amino]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 1342. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 1343. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 1344. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 1345. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 1346. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 1347. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 1348. | N-((1R,4R)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 1349. | N-((1S,4S)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 1350. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 1351. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 1352. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 1353. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 1354. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 1355. | N-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 1356. | N-(2-hydroxy-4-methanesulfonylphenyl)-2-methyl-N-[3-(4-{[(1R,4R)-4-{2-oxa-6- |
| | azaspiro[3.3]heptan-6-yl}cyclohexyl]amino}-1-(2,2,2-trifluoroethyl)-1H-indol-2-yl)prop- |
| | 2-yn-1-yl]propanamide |
| 1357. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)isobutyramide |
| 1358. | 2-(3-{[4-methanesulfonyl-2-(2-methoxyethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 1359. | 2-(3-{[4-methanesulfonyl-2-(2-methoxyethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 1360. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 1361. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 1362. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 1363. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 1364. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 1365. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)acetamide |
| 1366. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 1367. | 3-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1368. | 3-methoxy-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1369. | 2-(3-{[4-methanesulfonyl-2-(2,2,2-trifluoroethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 1370. | 2-(3-{[4-methanesulfonyl-2-(2,2,2-trifluoroethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 1371. | 2-[3-(2-amino-4-methanesulfonylphenoxy)prop-1-yn-1-yl]-N-[(1S,4S)-4-{2-oxa-6- |
| | azaspiro[3.3]heptan-6-yl}cyclohexyl]-1-(2,2,2-trifluoroethyl)-1H-indol-4-amine |
| 1372. | 2-{3-[(2-ethoxy-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 1373. | 2-{3-[(2-ethoxy-4-methanesulfonylphenyl)amino]prop-1-yn-1-yl}-N-[(1S,4S)-4-{2-oxa- |
| | 6-azaspiro[3.3]heptan-6-yl}cyclohexyl]-1-(2,2,2-trifluoroethyl)-1H-indol-4-amine |
| 1374. | 3-(2-methoxyethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1375. | 3-(2-methoxyethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1376. | N-ethyl-3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1377. | N-ethyl-3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1378. | 3-(2-fluoroethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1379. | 3-(2-fluoroethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1380. | 3-(cyanomethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1381. | 3-(cyanomethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1382. | 2-{3-[4-methanesulfonyl-2-(methylamino)phenoxy]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 1383. | 2-{3-[4-methanesulfonyl-2-(methylamino)phenoxy]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 1384. | 2-[3-(2-amino-4-methanesulfonylphenoxy)prop-1-yn-1-yl]-N-[(1R,4R)-4-{2-oxa-6- |
| | azaspiro[3.3]heptan-6-yl}cyclohexyl]-1-(2,2,2-trifluoroethyl)-1H-indol-4-amine |
| 1385. | N-((1R,4R)-4-(2-oxa-6-azaspiro[3.3]heptan-6-yl)cyclohexyl)-2-(3-(2-amino-4- |
| | (methylsulfonyl)phenoxy)prop-1-yn-1-yl)-1-(2,2,2-trifluoroethyl)-1H-indol-4-amine |
| 1386. | 3-hydroxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1387. | 3-methoxy-4-[(3-{4-[(4-{2-oxa-6-azaspiro[3.3]heptan-6-yl}cyclohexyl)amino]-1-(2,2,2- |
| | trifluoroethyl)-1H-indol-2-yl}prop-2-yn-1-yl)amino]benzonitrile |
| 1388. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzonitrile |
| 1389. | 2-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1390. | 2-(4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 1391. | 3-(fluoromethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1392. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(fluoromethoxy)-N- |
| | methylbenzamide |
| 1393. | 3-(2-cyanoethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1394. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(2-cyanoethoxy)-N- |
| | methylbenzamide |
| 1395. | 2-(3-{[2-(fluoromethoxy)-4-methanesulfonylphenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 1396. | N-((1S,4S)-4-(2-oxa-6-azaspiro[3.3]heptan-6-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1397. | 3-(cyanomethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1398. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3- |
| | (cyanomethoxy)benzenesulfonamide |
| 1399. | 3-(fluoromethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1400. | 3-(fluoromethoxy)-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1401. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1402. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1403. | N-((1R,4R)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 1404. | N-((1S,4S)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 1405. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 1406. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 1407. | N-((1R,4R)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1408. | N-((1S,4S)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1409. | N-((1R,4R)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 1410. | N-((1S,4S)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 1411. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1412. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzamide |
| 1413. | 2-fluoro-5-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1414. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxy-N- |
| | methylbenzamide |
| 1415. | 2-(3-{[2-methoxy-4-(trifluoromethyl)phenyl]amino}prop-1-yn-1-yl)-N-[(1R,4R)-4-{2- |
| | oxa-6-azaspiro[3.3]heptan-6-yl}cyclohexyl]-1-(2,2,2-trifluoroethyl)-1H-indol-4-amine |
| 1416. | N-((1S,4S)-4-(2-oxa-6-azaspiro[3.3]heptan-6-yl)cyclohexyl)-2-(3-((2-methoxy-4- |
| | (trifluoromethyl)phenyl)amino)prop-1-yn-1-yl)-1-(2,2,2-trifluoroethyl)-1H-indol-4-amine |
| 1417. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 1418. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 1419. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzoic acid |
| 1420. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzoic acid |
| 1421. | 2-{3-[(4-methanesulfonyl-2-methylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 1422. | N-((1S,4S)-4-(2-oxa-6-azaspiro[3.3]heptan-6-yl)cyclohexyl)-2-(3-((2-methyl-4- |
| | (methylsulfonyl)phenyl)amino)prop-1-yn-1-yl)-1-(2,2,2-trifluoroethyl)-1H-indol-4-amine |
| 1423. | 2-(3-{[4-methanesulfonyl-2-(trifluoromethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 1424. | N-((1S,4S)-4-(2-oxa-6-azaspiro[3.3]heptan-6-yl)cyclohexyl)-2-(3-((4-(methylsulfonyl)-2- |
| | (trifluoromethoxy)phenyl)amino)prop-1-yn-1-yl)-1-(2,2,2-trifluoroethyl)-1H-indol-4- |
| | amine |
| 1425. | 2-(3-{[4-methanesulfonyl-2-(trifluoromethyl)phenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 1426. | N-((1S,4S)-4-(2-oxa-6-azaspiro[3.3]heptan-6-yl)cyclohexyl)-2-(3-((4-(methylsulfonyl)-2- |
| | (trifluoromethyl)phenyl)amino)prop-1-yn-1-yl)-1-(2,2,2-trifluoroethyl)-1H-indol-4-amine |
| 1427. | 2-{3-[(2-chloro-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 1428. | N-((1S,4S)-4-(2-oxa-6-azaspiro[3.3]heptan-6-yl)cyclohexyl)-2-(3-((2-chloro-4- |
| | (methylsulfonyl)phenyl)amino)prop-1-yn-1-yl)-1-(2,2,2-trifluoroethyl)-1H-indol-4-amine |
| 1429. | 3-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 1430. | 3-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 1431. | 2-{3-[(4-methanesulfonyl-2-methoxyphenyl)amino]prop-1-yn-1-yl}-N-[(1S,4S)-4-{7- |
| | oxa-2-azaspiro[3.5]nonan-2-yl}cyclohexyl]-1-(2,2,2-trifluoroethyl)-1H-indol-4-amine |
| 1432. | 2-{3-[(4-methanesulfonyl-2-methoxyphenyl)amino]prop-1-yn-1-yl}-N-[(1R,4R)-4-{7- |
| | oxa-2-azaspiro[3.5]nonan-2-yl}cyclohexyl]-1-(2,2,2-trifluoroethyl)-1H-indol-4-amine |
| 1433. | 3-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1434. | 3-methoxy-N-methyl-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1435. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1436. | 3-methoxy-4-{[3-(4-{[(1S,4S)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1437. | 3-(cyanomethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1438. | 3-(cyanomethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1439. | 2-(3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1440. | 2-(4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 1441. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1442. | 2-(5-methanesulfonyl-2-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1443. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1444. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1445. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1446. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1447. | methyl 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetate |
| 1448. | methyl 2-(2-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetate |
| 1449. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetic acid |
| 1450. | 2-(2-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-5-(methylsulfonyl)phenoxy)acetic acid |
| 1451. | 2-{3-[(4-methanesulfonyl-2-methoxyphenyl)amino]prop-1-yn-1-yl}-N-[(1R,4R)-4-{2- |
| | oxa-7-azaspiro[3.5]nonan-7-yl}cyclohexyl]-1-(2,2,2-trifluoroethyl)-1H-indol-4-amine |
| 1452. | 2-(3-{[4-(ethanesulfonyl)-2-methoxyphenyl]amino}prop-1-yn-1-yl)-N-[(1R,4R)-4-{7- |
| | oxa-2-azaspiro[3.5]nonan-2-yl}cyclohexyl]-1-(2,2,2-trifluoroethyl)-1H-indol-4-amine |
| 1453. | 2-(3-{[4-(ethanesulfonyl)-2-methoxyphenyl]amino}prop-1-yn-1-yl)-N-[(1S,4S)-4-{7-oxa- |
| | 2-azaspiro[3.5]nonan-2-yl}cyclohexyl]-1-(2,2,2-trifluoroethyl)-1H-indol-4-amine |
| 1454. | 3-(fluoromethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1455. | 3-(fluoromethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1456. | 3-methoxy-N-methyl-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1457. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 1458. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1459. | 4-((3-(4-(((1S,4S)-4-(6-oxa-2-azaspiro[3.5]nonan-2-yl)cyclohexyl]amino)-1-(2,2,2- |
| | trifluoroethyl)-1H-indol-2-yl)prop-2-yn-1-yl)amino)-3-methoxybenzenesulfonamide |
| 1460. | 2-{3-[(4-methanesulfonyl-2-methoxyphenyl)amino]prop-1-yn-1-yl}-N-[(1R,4R)-4-{6- |
| | oxa-2-azaspiro[3.5]nonan-2-yl}cyclohexyl]-1-(2,2,2-trifluoroethyl)-1H-indol-4-amine |
| 1461. | N-((1S,4S)-4-(6-oxa-2-azaspiro[3.5]nonan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1462. | N-((1R,4R)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1463. | N-((1S,4S)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1464. | 4-(3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)-7-(methylsulfonyl)-2H- |
| | benzo[b][1,4]oxazin-3(4H)-one |
| 1465. | 4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1466. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1467. | N-((1R,4R)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1468. | N-((1S,4S)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1469. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1470. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1471. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1472. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1473. | 2-fluoro-5-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1474. | 2-(3-((2-methoxy-4-(methylsulfonyl)phenyl)amino)prop-1-yn-1-yl)-N-((1R,4R)-4-(3- |
| | methoxypyrrolidin-1-yl)cyclohexyl)-1-(2,2,2-trifluoroethyl)-1H-indol-4-amine |
| 1475. | 2-(3-((2-methoxy-4-(methylsulfonyl)phenyl)amino)prop-1-yn-1-yl)-N-((1S,4S)-4-(3- |
| | methoxypyrrolidin-1-yl)cyclohexyl)-1-(2,2,2-trifluoroethyl)-1H-indol-4-amine |
| 1476. | 3-methoxy-4-((3-(4-(((1R,4R)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1477. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1478. | 2-(3-{[2-(fluoromethoxy)-4-methanesulfonylphenyl]amino}prop-1-yn-1-yl)-N-[(1R,4R)- |
| | 4-(morpholin-4-yl)cyclohexyl]-1-(2,2,2-trifluoroethyl)-1H-indol-4-amine |
| 1479. | 2-(3-{[2-(fluoromethoxy)-4-methanesulfonylphenyl]amino}prop-1-yn-1-yl)-N-[(1S,4S)- |
| | 4-(morpholin-4-yl)cyclohexyl]-1-(2,2,2-trifluoroethyl)-1H-indol-4-amine |
| 1480. | 3-(cyanomethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1481. | 3-(cyanomethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1482. | 3-(fluoromethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1483. | 3-(fluoromethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1484. | 2-(3-((2-ethoxy-4-(methylsulfonyl)phenyl)amino)prop-1-yn-1-yl)-N-((1R,4R)-4- |
| | morpholinocyclohexyl)-1-(2,2,2-trifluoroethyl)-1H-indol-4-amine |
| 1485. | 2-(3-((2-ethoxy-4-(methylsulfonyl)phenyl)amino)prop-1-yn-1-yl)-N-((1S,4S)-4- |
| | morpholinocyclohexyl)-1-(2,2,2-trifluoroethyl)-1H-indol-4-amine |
| 1486. | 2-{3-[(4-methanesulfonyl-2-methoxyphenyl)amino]prop-1-yn-1-yl}-N-[(1R,4R)-4-(3- |
| | methoxypiperidin-1-yl)cyclohexyl]-1-(2,2,2-trifluoroethyl)-1H-indol-4-amine |
| 1487. | 2-(3-((2-methoxy-4-(methylsulfonyl)phenyl)amino)prop-1-yn-1-yl)-N-((1S,4S)-4-(3- |
| | methoxypiperidin-1-yl)cyclohexyl)-1-(2,2,2-trifluoroethyl)-1H-indol-4-amine |
| 1488. | 2-{3-[(4-methanesulfonyl-2-methoxyphenyl)amino]prop-1-yn-1-yl}-N-[(1R,4R)-4-(4- |
| | methoxypiperidin-1-yl)cyclohexyl]-1-(2,2,2-trifluoroethyl)-1H-indol-4-amine |
| 1489. | 2-(3-((2-methoxy-4-(methylsulfonyl)phenyl)amino)prop-1-yn-1-yl)-N-((1S,4S)-4-(4- |
| | methoxypiperidin-1-yl)cyclohexyl)-1-(2,2,2-trifluoroethyl)-1H-indol-4-amine |
| 1490. | 3-methoxy-N-methyl-4-{[3-(4-{[(1R,4R)-4-(4-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1491. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1492. | 3-methoxy-4-{[3-(4-{[(1R,4R)-4-(4-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1493. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1494. | 3-methoxy-N-methyl-4-{[3-(4-{[(1R,4R)-4-(3-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1495. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1496. | 3-methoxy-4-{[3-(4-{[(1R,4R)-4-(3-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1497. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1498. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 1499. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 1500. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁶-thiomorpholine-1,1-dione |
| 1501. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1,1-dioxide |
| 1502. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1503. | 4-[(1S,4S)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1504. | 4-((3-(4-(((1R,4R)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1505. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1506. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1-oxide |
| 1507. | 3-methoxy-N-methyl-4-{[3-(4-{[(1R,4R)-4-(1-oxo-1λ⁴-thiomorpholin-4- |
| | yl)cyclohexyl]amino}-1-(2,2,2-trifluoroethyl)-1H-indol-2-yl)prop-2-yn-1- |
| | yl]amino}benzamide |
| 1508. | 3-methoxy-4-{[3-(4-{[(1R,4R)-4-(1-oxo-1λ⁴-thiomorpholin-4-yl)cyclohexyl]amino}-1- |
| | (2,2,2-trifluoroethyl)-1H-indol-2-yl)prop-2-yn-1-yl]amino}benzene-1-sulfonamide |
| 1509. | 3-methoxy-4-((3-(4-(((1S,4S)-4-(1-oxidothiomorpholino)cyclohexyl)amino)-1-(2,2,2- |
| | trifluoroethyl)-1H-indol-2-yl)prop-2-yn-1-yl)amino)benzenesulfonamide |
| 1510. | 3-methoxy-4-{[3-(4-{[(1R,4R)-4-(1,1-dioxo-1λ⁶-thiomorpholin-4-yl)cyclohexyl]amino}- |
| | 1-(2,2,2-trifluoroethyl)-1H-indol-2-yl)prop-2-yn-1-yl]amino}benzene-1-sulfonamide |
| 1511. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxybenzenesulfonamide |
| 1512. | 2-{3-[(4-methanesulfonyl-2-methoxyphenyl)amino]prop-1-yn-1-yl}-N-[(1S,4S)-4-{2- |
| | oxa-7-azaspiro[3.5]nonan-7-yl}cyclohexyl]-1-(2,2,2-trifluoroethyl)-1H-indol-4-amine |
| 1513. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1514. | 3-methoxy-N-methyl-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1515. | 3-methoxy-4-{[3-(4-{[(1R,4R)-4-{2-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1516. | 4-((3-(4-(((1S,4S)-4-(2-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1517. | 3-(cyanomethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1518. | 3-(cyanomethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1519. | 3-(fluoromethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1520. | 3-(fluoromethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1521. | 2-{3-[(4-methanesulfonyl-2-methoxyphenyl)amino]prop-1-yn-1-yl}-N-[(1R,4R)-4-{1- |
| | oxa-7-azaspiro[3.5]nonan-7-yl}cyclohexyl]-1-(2,2,2-trifluoroethyl)-1H-indol-4-amine |
| 1522. | N-((1S,4S)-4-(1-oxa-7-azaspiro[3.5]nonan-7-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1523. | 3-methoxy-N-methyl-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1524. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxy-N-methylbenzamide |
| 1525. | 3-methoxy-4-{[3-(4-{[(1R,4R)-4-{1-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1526. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1527. | 2-(5-methanesulfonyl-2-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1528. | 2-(5-methanesulfonyl-2-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1529. | 2-(3-{[2-(fluoromethoxy)-4-methanesulfonylphenyl]amino}prop-1-yn-1-yl)-N-[(1R,4R)- |
| | 4-{2-oxa-7-azaspiro[3.5]nonan-7-yl}cyclohexyl]-1-(2,2,2-trifluoroethyl)-1H-indol-4- |
| | amine |
| 1530. | 2-(3-{[2-(fluoromethoxy)-4-methanesulfonylphenyl]amino}prop-1-yn-1-yl)-N-[(1S,4S)- |
| | 4-{2-oxa-7-azaspiro[3.5]nonan-7-yl}cyclohexyl]-1-(2,2,2-trifluoroethyl)-1H-indol-4- |
| | amine |
| 1531. | N-((1R,4R)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1532. | N-((1S,4S)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1533. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1534. | N-((1R,4R)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1535. | N-((1S,4S)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1536. | 2-{3-[(4-methanesulfonyl-2-methoxyphenyl)amino]prop-1-yn-1-yl}-N-[(1R,4R)-4-{6- |
| | oxa-3-azabicyclo[3.1.1]heptan-3-yl}cyclohexyl]-1-(2,2,2-trifluoroethyl)-1H-indol-4- |
| | amine |
| 1537. | 2-{3-[(4-methanesulfonyl-2-methoxyphenyl)amino]prop-1-yn-1-yl}-N-[(1S,4S)-4-{6- |
| | oxa-3-azabicyclo[3.1.1]heptan-3-yl}cyclohexyl]-1-(2,2,2-trifluoroethyl)-1H-indol-4- |
| | amine |
| 1538. | 3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1539. | 3-methoxy-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1540. | 3-methoxy-N-methyl-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1541. | 3-methoxy-N-methyl-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1542. | 2-{3-[(2-ethoxy-4-methanesulfonylphenyl)amino]prop-1-yn-1-yl}-N-[(1R,4R)-4-{6-oxa- |
| | 3-azabicyclo[3.1.1]heptan-3-yl}cyclohexyl]-1-(2,2,2-trifluoroethyl)-1H-indol-4-amine |
| 1543. | 2-{3-[(2-ethoxy-4-methanesulfonylphenyl)amino]prop-1-yn-1-yl}-N-[(1S,4S)-4-{6-oxa- |
| | 3-azabicyclo[3.1.1]heptan-3-yl}cyclohexyl]-1-(2,2,2-trifluoroethyl)-1H-indol-4-amine |
| 1544. | N-ethyl-3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1545. | 2-(5-methanesulfonyl-2-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}phenoxy)acetonitrile |
| 1546. | 2-(2-((3-(4-(((1S,4S)-4-(6-oxa-3-azabicyclo[3.1.1]heptan-3-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetonitrile |
| 1547. | 3-methoxy-N-methyl-4-{[3-(4-{[(1R,4R)-4-{2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzamide |
| 1548. | 2-{3-[(4-methanesulfonyl-2-methoxyphenyl)amino]prop-1-yn-1-yl}-N-[(1R,4R)-4-{2- |
| | oxa-8-azaspiro[4.5]decan-8-yl}cyclohexyl]-1-(2,2,2-trifluoroethyl)-1H-indol-4-amine |
| 1549. | 3-methoxy-4-{[3-(4-{[(1R,4R)-4-{2-oxa-8-azaspiro[4.5]decan-8-yl}cyclohexyl]amino}- |
| | 1-(2,2,2-trifluoroethyl)-1H-indol-2-yl)prop-2-yn-1-yl]amino}benzene-1-sulfonamide |
| 1550. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1551. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1552. | 2-{3-[(4-methanesulfonyl-2-methoxyphenyl)amino]prop-1-yn-1-yl}-N-[(1R,4R)-4-{3- |
| | oxa-9-azaspiro[5.5]undecan-9-yl}cyclohexyl]-1-(2,2,2-trifluoroethyl)-1H-indol-4-amine |
| 1553. | N-((1S,4S)-4-(3-oxa-9-azaspiro[5.5]undecan-9-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1554. | 3-methoxy-N-methyl-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzamide |
| 1555. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxy-N-methylbenzamide |
| 1556. | 3-methoxy-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzene-1-sulfonamide |
| 1557. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxybenzenesulfonamide |
| 1558. | 3-methoxy-N-methyl-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzamide |
| 1559. | 3-methoxy-N-methyl-4-((3-(4-(((1S,4S)-4-(pyrrolidin-1-yl)cyclohexyl]amino)-1-(2,2,2- |
| | trifluoroethyl)-1H-indol-2-yl)prop-2-yn-1-yl)amino)benzamide |
| 1560. | 3-methoxy-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1561. | 3-methoxy-4-((3-(4-(((1S,4S)-4-(pyrrolidin-1-yl)cyclohexyl]amino)-1-(2,2,2- |
| | trifluoroethyl)-1H-indol-2-yl)prop-2-yn-1-yl)amino)benzenesulfonamide |
| 1562. | 2-{3-[(4-methanesulfonyl-2-methoxyphenyl)amino]prop-1-yn-1-yl}-N-[(1R,4R)-4- |
| | (pyrrolidin-1-yl)cyclohexyl]-1-(2,2,2-trifluoroethyl)-1H-indol-4-amine |
| 1563. | 2-(3-((2-methoxy-4-(methylsulfonyl)phenyl)amino)prop-1-yn-1-yl)-N-((1S,4S)-4- |
| | (pyrrolidin-1-yl)cyclohexyl)-1-(2,2,2-trifluoroethyl)-1H-indol-4-amine |
| 1564. | 2-{3-[(4-methanesulfonyl-2-methoxyphenyl)amino]prop-1-yn-1-yl}-N-[(1R,4R)-4-(3- |
| | methoxypyrrolidin-1-yl)cyclohexyl]-1-(2,2,2-trifluoroethyl)-1H-indol-4-amine |
| 1565. | 3-methoxy-4-{[3-(4-{[(1R,4R)-4-(3-methoxypyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1566. | 3-methoxy-N-methyl-4-{[3-(4-{[(1R,4R)-4-(3-methoxypyrrolidin-1- |
| | yl)cyclohexyl]amino}-1-(2,2,2-trifluoroethyl)-1H-indol-2-yl)prop-2-yn-1- |
| | yl]amino}benzamide |
| 1567. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)-N-methylbenzamide |
| 1568. | 3-methoxy-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzene-1-sulfonamide |
| 1569. | 3-methoxy-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzenesulfonamide |
| 1570. | 2-{3-[(4-methanesulfonyl-2-methoxyphenyl)amino]prop-1-yn-1-yl}-N-[(1R,4R)-4- |
| | {hexahydro-1H-furo[3,4-c]pyrrol-5-yl}cyclohexyl]-1-(2,2,2-trifluoroethyl)-1H-indol-4- |
| | amine |
| 1571. | 2-(3-((2-methoxy-4-(methylsulfonyl)phenyl)amino)prop-1-yn-1-yl)-N-((1R,4R)-4- |
| | (tetrahydro-1H-furo[3,4-c]pyrrol-5(3H)-yl)cyclohexyl)-1-(2,2,2-trifluoroethyl)-1H-indol- |
| | 4-amine |
| 1572. | 3-methoxy-N-methyl-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzamide |
| 1573. | 3-methoxy-N-methyl-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzamide |
| 1574. | 2-{3-[(4-methanesulfonyl-2-methoxyphenyl)amino]prop-1-yn-1-yl}-N-[(1R,4R)-4-[4- |
| | (trifluoromethyl)piperidin-1-yl]cyclohexyl]-1-(2,2,2-trifluoroethyl)-1H-indol-4-amine |
| 1575. | 2-(3-((2-methoxy-4-(methylsulfonyl)phenyl)amino)prop-1-yn-1-yl)-1-(2,2,2- |
| | trifluoroethyl)-N-((1S,4S)-4-(4-(trifluoromethyl)piperidin-1-yl)cyclohexyl)-1H-indol-4- |
| | amine |
| 1576. | 3-methoxy-N-methyl-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzamide |
| 1577. | 3-methoxy-N-methyl-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzamide |
| 1578. | 3-methoxy-4-{[3-(4-{[(1R,4R)-4-[4-(trifluoromethyl)piperidin-1-yl]cyclohexyl]amino}- |
| | 1-(2,2,2-trifluoroethyl)-1H-indol-2-yl)prop-2-yn-1-yl]amino}benzene-1-sulfonamide |
| 1579. | 3-methoxy-4-((3-(1-(2,2,2-trifluoroethyl)-4-(((1S,4S)-4-(4-(trifluoromethyl)piperidin-1- |
| | yl)cyclohexyl)amino)-1H-indol-2-yl)prop-2-yn-1-yl)amino)benzenesulfonamide |
| 1580. | 2-{3-[(4-methanesulfonyl-2-methoxyphenyl)amino]prop-1-yn-1-yl}-N-[(1R,4R)-4-(4- |
| | methanesulfonylpiperidin-1-yl)cyclohexyl]-1-(2,2,2-trifluoroethyl)-1H-indol-4-amine |
| 1581. | 2-(3-((2-methoxy-4-(methylsulfonyl)phenyl)amino)prop-1-yn-1-yl)-N-((1S,4S)-4-(4- |
| | (methylsulfonyl)piperidin-1-yl)cyclohexyl)-1-(2,2,2-trifluoroethyl)-1H-indol-4-amine |
| 1582. | 3-methoxy-N-methyl-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzamide |
| 1583. | 3-methoxy-N-methyl-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzamide |
| 1584. | 3-methoxy-4-{[3-(4-{[(1R,4R)-4-(4-methanesulfonylpiperidin-1-yl)cyclohexyl]amino}-1- |
| | (2,2,2-trifluoroethyl)-1H-indol-2-yl)prop-2-yn-1-yl]amino}benzene-1-sulfonamide |
| 1585. | 3-methoxy-4-((3-(4-(((1S,4S)-4-(4-(methylsulfonyl)piperidin-1-yl)cyclohexyl)amino)-1- |
| | (2,2,2-trifluoroethyl)-1H-indol-2-yl)prop-2-yn-1-yl)amino)benzenesulfonamide |
| 1586. | 1-[(1S,3R)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-4,5-dihydro-1H-1,2,3,4-tetrazol- |
| | 5-one |
| 1587. | 2-((2-(3-((2-methoxy-4-(methylsulfonyl)phenyl)amino)prop-1-yn-1-yl)-4-(((1R,4R)-4- |
| | morpholinocyclohexyl)amino)-1H-indol-1-yl)methyl)acrylonitrile |
| 1588. | N-((1R,4R)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1589. | N-((1S,4S)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1590. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1591. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1592. | 1-{3-[(2-{3-[(4-methanesulfonyl-2-methoxyphenyl)amino]prop-1-yn-1-yl}-1-(2,2,2- |
| | trifluoroethyl)-1H-indol-4-yl)amino]pyrrolidin-1-yl}-3-methoxypropan-2-ol |
| 1593. | N-[3-(4-{[1-(2-hydroxy-3-methoxypropyl)pyrrolidin-3-yl]amino}-1-(2,2,2- |
| | trifluoroethyl)-1H-indol-2-yl)prop-2-yn-1-yl]benzamide |
| 1594. | 1-{3-[(2-{3-[(4-methanesulfonylphenyl)amino]prop-1-yn-1-yl}-1-(2,2,2-trifluoroethyl)- |
| | 1H-indol-4-yl)amino]pyrrolidin-1-yl}-3-methoxypropan-2-ol |
| 1595. | 2-{3-[(4-methanesulfonyl-2-methoxyphenyl)amino]prop-1-yn-1-yl}-N-[1-(oxan-4- |
| | yl)pyrrolidin-3-yl]-1-(2,2,2-trifluoroethyl)-1H-indol-4-amine |
| 1596. | 2-(3-{[2-methoxy-4-(morpholine-4-sulfonyl)phenyl]amino}prop-1-yn-1-yl)-N-(piperidin- |
| | 4-yl)-1-(2,2,2-trifluoroethyl)-1H-indol-4-amine |
| 1597. | 3-methoxy-N,N-dimethyl-4-[(3-{4-[(piperidin-4-yl)amino]-1-(2,2,2-trifluoroethyl)-1H- |
| | indol-2-yl}prop-2-yn-1-yl)amino]benzene-1-sulfonamide |
| 1598. | 2-{5-methanesulfonyl-2-[(3-{4-[(piperidin-4-yl)amino]-1-(2,2,2-trifluoroethyl)-1H-indol- |
| | 2-yl}prop-2-yn-1-yl)amino]phenoxy}acetonitrile |
| 1599. | 3-methoxy-4-((3-(4-(piperidin-4-ylamino)-1-(2,2,2-trifluoroethyl)-1H-indol-2-yl)prop-2- |
| | yn-1-yl)amino)benzamide |
| 1600. | 2-{3-[(4-methanesulfonyl-2-methoxyphenyl)amino]prop-1-yn-1-yl}-N-[(3R)-piperidin-3- |
| | yl]-1-(2,2,2-trifluoroethyl)-1H-indol-4-amine |
| 1601. | 3-methoxy-4-{[3-(4-{[(2S,4S)-2-methylpiperidin-4-yl]amino}-1-(2,2,2-trifluoroethyl)- |
| | 1H-indol-2-yl)prop-2-yn-1-yl]amino}benzene-1-sulfonamide |
| 1602. | 3-methoxy-4-((3-(4-((1-methylpiperidin-4-yl)amino)-1-(2,2,2-trifluoroethyl)-1H-indol-2- |
| | yl)prop-2-yn-1-yl)amino)benzenesulfonamide |
| 1603. | 2-(3-{[4-methanesulfonyl-2-(trifluoromethoxy)phenyl]amino}prop-1-yn-1-yl)-N-(1- |
| | methylpiperidin-4-yl)-1-(2,2,2-trifluoroethyl)-1H-indol-4-amine |
| 1604. | N-(1-methylpiperidin-4-yl)-2-(3-((4-(methylsulfonyl)-2-(2,2,2- |
| | trifluoroethoxy)phenyl)amino)prop-1-yn-1-yl)-1-(2,2,2-trifluoroethyl)-1H-indol-4-amine |
| 1605. | 2-{4-methoxy-5-[(3-{4-[(1-methylpiperidin-4-yl)amino]-1-(2,2,2-trifluoroethyl)-1H- |
| | indol-2-yl}prop-2-yn-1-yl)amino]pyridin-2-yl}-2-methylpropanenitrile |
| 1606. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-N-(1,2-oxazol-3-yl)benzene-1-sulfonamide |
| 1607. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-N-(oxan-4-yl)benzene-1-sulfonamide |
| 1608. | 3-methoxy-N-(5-methyl-1,2-oxazol-3-yl)-4-[(3-{4-[(1-methylpiperidin-4-yl)amino]-1- |
| | (2,2,2-trifluoroethyl)-1H-indol-2-yl}prop-2-yn-1-yl)amino]benzene-1-sulfonamide |
| 1609. | N-(2-hydroxyethyl)-3-methoxy-N-methyl-4-[(3-{4-[(1-methylpiperidin-4-yl)amino]-1- |
| | (2,2,2-trifluoroethyl)-1H-indol-2-yl}prop-2-yn-1-yl)amino]benzene-1-sulfonamide |
| 1610. | 3-methoxy-N,N-dimethyl-4-[(3-{4-[(1-methylpiperidin-4-yl)amino]-1-(2,2,2- |
| | trifluoroethyl)-1H-indol-2-yl}prop-2-yn-1-yl)amino]benzene-1-sulfonamide |
| 1611. | 4-[(3-{6-fluoro-4-[(1-methylpiperidin-4-yl)amino]-1-(2,2,2-trifluoroethyl)-1H-indol-2- |
| | yl}prop-2-yn-1-yl)amino]-3-methoxybenzene-1-sulfonamide |
| 1612. | 6-fluoro-2-{3-[(4-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 1613. | 2-(3-((4-methoxy-6-(methylsulfonyl)pyridin-3-yl)amino)prop-1-yn-1-yl)-N-(1- |
| | methylpiperidin-4-yl)-1-(2,2,2-trifluoroethyl)-1H-indol-4-amine |
| 1614. | 3-methoxy-N-(2-methoxyethyl)-N-methyl-4-[(3-{4-[(1-methylpiperidin-4-yl)amino]-1- |
| | (2,2,2-trifluoroethyl)-1H-indol-2-yl}prop-2-yn-1-yl)amino]benzene-1-sulfonamide |
| 1615. | 2-{3-[(4-methanesulfonyl-2-methoxyphenyl)(methyl)amino]prop-1-yn-1-yl}-N-(1- |
| | methylpiperidin-4-yl)-1-(2,2,2-trifluoroethyl)-1H-indol-4-amine |
| 1616. | 1-(4-{3-methoxy-4-[(3-{4-[(1-methylpiperidin-4-yl)amino]-1-(2,2,2-trifluoroethyl)-1H- |
| | indol-2-yl}prop-2-yn-1-yl)amino]benzenesulfonyl}piperazin-1-yl)ethan-1-one |
| 1617. | 2-(3-{[2-methoxy-4-(morpholine-4-sulfonyl)phenyl]amino}prop-1-yn-1-yl)-N-(1- |
| | methylpiperidin-4-yl)-1-(2,2,2-trifluoroethyl)-1H-indol-4-amine |
| 1618. | 3-methoxy-4-((3-(4-((1-methylpiperidin-4-yl)amino)-1-(2,2,2-trifluoroethyl)-1H-indol-2- |
| | yl)prop-2-yn-1-yl)amino)benzoic acid |
| 1619. | 3-methoxy-N-methyl-4-[(3-{4-[(1-methylpiperidin-4-yl)amino]-1-(2,2,2-trifluoroethyl)- |
| | 1H-indol-2-yl}prop-2-yn-1-yl)amino]benzamide |
| 1620. | N,N-bis(2-hydroxyethyl)-3-methoxy-4-[(3-{4-[(1-methylpiperidin-4-yl)amino]-1-(2,2,2- |
| | trifluoroethyl)-1H-indol-2-yl}prop-2-yn-1-yl)amino]benzene-1-sulfonamide |
| 1621. | 3-methoxy-N-methyl-4-[(3-{4-[(1-methylpiperidin-4-yl)amino]-1-(2,2,2-trifluoroethyl)- |
| | 1H-indol-2-yl}prop-2-yn-1-yl)amino]benzene-1-sulfonamide |
| 1622. | 2-[3-({2-methoxy-4-[(4-methylpiperazin-1-yl)sulfonyl]phenyl}amino)prop-1-yn-1-yl]-N- |
| | (1-methylpiperidin-4-yl)-1-(2,2,2-trifluoroethyl)-1H-indol-4-amine |
| 1623. | 5-methanesulfonyl-2-[(3-{4-[(1-methylpiperidin-4-yl)amino]-1-(2,2,2-trifluoroethyl)-1H- |
| | indol-2-yl}prop-2-yn-1-yl)amino]phenol |
| 1624. | 2-{3-[(4-methanesulfonyl-2-methoxyphenyl)amino]prop-1-yn-1-yl}-6-methoxy-N-(1- |
| | methylpiperidin-4-yl)-1-(2,2,2-trifluoroethyl)-1H-indol-4-amine |
| 1625. | 2-{3-[2-(dimethylamino)-4-methanesulfonylphenoxy]prop-1-yn-1-yl}-N-(1- |
| | methylpiperidin-4-yl)-1-(2,2,2-trifluoroethyl)-1H-indol-4-amine |
| 1626. | 2-{5-methanesulfonyl-2-[(3-{4-[(1-methylpiperidin-4-yl)amino]-1-(2,2,2-trifluoroethyl)- |
| | 1H-indol-2-yl}prop-2-yn-1-yl)amino]phenoxy}acetonitrile |
| 1627. | 2-(3-{[2-(fluoromethoxy)-4-methanesulfonylphenyl]amino}prop-1-yn-1-yl)-N-(1- |
| | methylpiperidin-4-yl)-1-(2,2,2-trifluoroethyl)-1H-indol-4-amine |
| 1628. | 2-(3-{[2-methoxy-4-(morpholine-4-carbonyl)phenyl]amino}prop-1-yn-1-yl)-N-(1- |
| | methylpiperidin-4-yl)-1-(2,2,2-trifluoroethyl)-1H-indol-4-amine |
| 1629. | 1-{3-methoxy-4-[(3-{4-[(1-methylpiperidin-4-yl)amino]-1-(2,2,2-trifluoroethyl)-1H- |
| | indol-2-yl}prop-2-yn-1-yl)amino]benzoyl}piperidin-4-ol |
| 1630. | 3-(3-(4-((1-methylpiperidin-4-yl)amino)-1-(2,2,2-trifluoroethyl)-1H-indol-2-yl)prop-2- |
| | yn-1-yl)-6-(methylsulfonyl)benzo[d]oxazol-2(3H)-one |
| 1631. | 2-(3-{[2-methoxy-4-(5-methoxypyridin-3-yl)phenyl]amino}prop-1-yn-1-yl)-N-(1- |
| | methylpiperidin-4-yl)-1-(2,2,2-trifluoroethyl)-1H-indol-4-amine |
| 1632. | 2-{3-[(5-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 1633. | N-(2-hydroxyethyl)-3-methoxy-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1634. | 3-methoxy-N-(2-methoxyethyl)-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1635. | 3-methoxy-N-(1-methylpiperidin-4-yl)-4-[(3-{4-[(1-methylpiperidin-4-yl)amino]-1- |
| | (2,2,2-trifluoroethyl)-1H-indol-2-yl}prop-2-yn-1-yl)amino]benzamide |
| 1636. | 2-[3-({4-[4-(dimethylamino)piperidine-1-carbonyl]-2-methoxyphenyl}amino)prop-1-yn- |
| | 1-yl]-N-(1-methylpiperidin-4-yl)-1-(2,2,2-trifluoroethyl)-1H-indol-4-amine |
| 1637. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-N-(oxan-4-yl)benzamide |
| 1638. | 2-(3-{[2-methoxy-4-(4-methylpiperazine-1-carbonyl)phenyl]amino}prop-1-yn-1-yl)-N- |
| | (1-methylpiperidin-4-yl)-1-(2,2,2-trifluoroethyl)-1H-indol-4-amine |
| 1639. | 2-{3-[(2-methoxy-4-{2-oxa-6-azaspiro[3.3]heptane-6-carbonyl}phenyl)amino]prop-1-yn- |
| | 1-yl}-N-(1-methylpiperidin-4-yl)-1-(2,2,2-trifluoroethyl)-1H-indol-4-amine |
| 1640. | 2-(3-{[2-methoxy-4-(pyridin-3-yl)phenyl]amino}prop-1-yn-1-yl)-N-(1-methylpiperidin- |
| | 4-yl)-1-(2,2,2-trifluoroethyl)-1H-indol-4-amine |
| 1641. | 2-(3-{[2-methoxy-4-(pyridin-4-yl)phenyl]amino}prop-1-yn-1-yl)-N-(1-methylpiperidin- |
| | 4-yl)-1-(2,2,2-trifluoroethyl)-1H-indol-4-amine |
| 1642. | N-(3-{4-[(1-methylpiperidin-4-yl)amino]-1-(2,2,2-trifluoroethyl)-1H-indol-2-yl}prop-2- |
| | yn-1-yl)pyridine-3-carboxamide |
| 1643. | 2-(3-{[2-methoxy-4-(1,3-oxazol-2-yl)phenyl]amino}prop-1-yn-1-yl)-N-(1- |
| | methylpiperidin-4-yl)-1-(2,2,2-trifluoroethyl)-1H-indol-4-amine |
| 1644. | 2-{3-[(3-methanesulfonylphenyl)amino]prop-1-yn-1-yl}-N-(1-methylpiperidin-4-yl)-1- |
| | (2,2,2-trifluoroethyl)-1H-indol-4-amine |
| 1645. | N-(1-methylpiperidin-4-yl)-2-[3-({4-[(morpholin-4-yl)methyl]phenyl}amino)prop-1-yn- |
| | 1-yl]-1-(2,2,2-trifluoroethyl)-1H-indol-4-amine |
| 1646. | 2-(3-{[2-methoxy-4-(1,3-thiazol-2-yl)phenyl]amino}prop-1-yn-1-yl)-N-(1- |
| | methylpiperidin-4-yl)-1-(2,2,2-trifluoroethyl)-1H-indol-4-amine |
| 1647. | 2-[3-({2-methoxy-4-[1-(2-methoxyethyl)-1H-pyrazol-4-yl]phenyl}amino)prop-1-yn-1- |
| | yl]-N-(1-methylpiperidin-4-yl)-1-(2,2,2-trifluoroethyl)-1H-indol-4-amine |
| 1648. | 2-{3-[(2-methoxy-4-{7-oxa-2-azaspiro[3.5]nonane-2-carbonyl}phenyl)amino]prop-1-yn- |
| | 1-yl}-N-(1-methylpiperidin-4-yl)-1-(2,2,2-trifluoroethyl)-1H-indol-4-amine |
| 1649. | 2-{3-[(4-chloro-2-methoxyphenyl)amino]prop-1-yn-1-yl}-N-(1-methylpiperidin-4-yl)-1- |
| | (2,2,2-trifluoroethyl)-1H-indol-4-amine |
| 1650. | 2-(3-(((3S,4R)-3-methoxytetrahydro-2H-pyran-4-yl)amino)prop-1-yn-1-yl)-N-(1- |
| | methylpiperidin-4-yl)-1-(2,2,2-trifluoroethyl)-1H-indol-4-amine |
| 1651. | 2-fluoro-5-methoxy-4-[(3-{4-[(1-methylpiperidin-4-yl)amino]-1-(2,2,2-trifluoroethyl)- |
| | 1H-indol-2-yl}prop-2-yn-1-yl)amino]benzamide |
| 1652. | 2-(3-{[2-methoxy-4-(trifluoromethyl)phenyl]amino}prop-1-yn-1-yl)-N-(1- |
| | methylpiperidin-4-yl)-1-(2,2,2-trifluoroethyl)-1H-indol-4-amine |
| 1653. | 2-fluoro-5-methoxy-N-methyl-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1654. | 2-{3-[(4-methanesulfonyl-2-methoxyphenyl)amino]prop-1-yn-1-yl}-N-(1- |
| | methylpiperidin-4-yl)-1-[(oxiran-2-yl)methyl]-1H-indol-4-amine |
| 1655. | 2-(3-((2-ethoxy-4-(methylsulfonyl)phenyl)amino)prop-1-yn-1-yl)-N-(1-methylpiperidin- |
| | 4-yl)-1-(2,2,2-trifluoroethyl)-1H-indol-4-amine |
| 1656. | 2-{3-[(2-fluoro-4-methanesulfonyl-6-methoxyphenyl)amino]prop-1-yn-1-yl}-N-(1- |
| | methylpiperidin-4-yl)-1-(2,2,2-trifluoroethyl)-1H-indol-4-amine |
| 1657. | 2-{3-[(4-methanesulfonyl-2-methoxyphenyl)amino]prop-1-yn-1-yl}-3-methyl-N-(1- |
| | methylpiperidin-4-yl)-1-(2,2,2-trifluoroethyl)-1H-indol-4-amine |
| 1658. | 2-(3-(((3R,4R)-3-methoxytetrahydro-2H-pyran-4-yl)amino)prop-1-yn-1-yl)-N-(1- |
| | methylpiperidin-4-yl)-1-(2,2,2-trifluoroethyl)-1H-indol-4-amine |
| 1659. | 2-{3-[(5-fluoro-4-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 1660. | 2-{3-[(5-methanesulfonylthiophen-2-yl)amino]prop-1-yn-1-yl}-N-(1-methylpiperidin-4- |
| | yl)-1-(2,2,2-trifluoroethyl)-1H-indol-4-amine |
| 1661. | N-methyl-5-[(3-{4-[(1-methylpiperidin-4-yl)amino]-1-(2,2,2-trifluoroethyl)-1H-indol-2- |
| | yl}prop-2-yn-1-yl)amino]thiophene-2-carboxamide |
| 1662. | N,N-dimethyl-5-[(3-{4-[(1-methylpiperidin-4-yl)amino]-1-(2,2,2-trifluoroethyl)-1H- |
| | indol-2-yl}prop-2-yn-1-yl)amino]thiophene-2-carboxamide |
| 1663. | 5-[(3-{4-[(1-methylpiperidin-4-yl)amino]-1-(2,2,2-trifluoroethyl)-1H-indol-2-yl}prop-2- |
| | yn-1-yl)amino]thiophene-2-carboxylic acid |
| 1664. | 2-(3-((4-methoxypyridin-3-yl)amino)prop-1-yn-1-yl)-N-(1-methylpiperidin-4-yl)-1- |
| | (2,2,2-trifluoroethyl)-1H-indol-4-amine |
| 1665. | 2-(2-((3-(4-((1-methylpiperidin-4-yl)amino)-1-(2,2,2-trifluoroethyl)-1H-indol-2-yl)prop- |
| | 2-yn-1-yl)amino)-5-(methylsulfonyl)phenoxy)acetamide |
| 1666. | N-(2-hydroxyethyl)-3-methoxy-4-((3-(4-((1-methylpiperidin-4-yl)amino)-1-(2,2,2- |
| | trifluoroethyl)-1H-indol-2-yl)prop-2-yn-1-yl)amino)benzenesulfonamide |
| 1667. | 4-((2-(3-((2-methoxy-4-(methylsulfonyl)phenyl)amino)prop-1-yn-1-yl)-1-(2,2,2- |
| | trifluoroethyl)-1H-indol-4-yl)amino)-1-methylpiperidin-2-one |
| 1668. | 2-{3-[(4-methanesulfonyl-2-methoxyphenyl)amino]prop-1-yn-1-yl}-N-[(3R,4S)-3- |
| | methoxy-1-methylpiperidin-4-yl]-1-(2,2,2-trifluoroethyl)-1H-indol-4-amine |
| 1669. | 2-{3-[(4-methanesulfonyl-2-methoxyphenyl)amino]prop-1-yn-1-yl}-N-[(3S,4R)-3- |
| | methoxy-1-methylpiperidin-4-yl]-1-(2,2,2-trifluoroethyl)-1H-indol-4-amine |
| 1670. | 2-{3-[(4-methanesulfonyl-2-methoxyphenyl)amino]prop-1-yn-1-yl}-N-[(3S,4R)-3- |
| | methoxy-1-methylpiperidin-4-yl]-N-methyl-1-(2,2,2-trifluoroethyl)-1H-indol-4-amine |
| 1671. | N-(1-ethylpiperidin-4-yl)-2-(3-{[2-(fluoromethoxy)-4- |
| | methanesulfonylphenyl]amino}prop-1-yn-1-yl)-1-(2,2,2-trifluoroethyl)-1H-indol-4-amine |
| 1672. | 4-[(3-{4-[(1-ethylpiperidin-4-yl)amino]-1-(2,2,2-trifluoroethyl)-1H-indol-2-yl}prop-2-yn- |
| | 1-yl)amino]-3-methoxy-N-methylbenzamide |
| 1673. | 2-{2-[(3-{4-[(1-ethylpiperidin-4-yl)amino]-1-(2,2,2-trifluoroethyl)-1H-indol-2-yl}prop-2- |
| | yn-1-yl)amino]-5-methanesulfonylphenoxy}acetonitrile |
| 1674. | N-(1-ethylpiperidin-4-yl)-2-{3-[(4-methanesulfonyl-2-methoxyphenyl)amino]prop-1-yn- |
| | 1-yl}-1-(2,2,2-trifluoroethyl)-1H-indol-4-amine |
| 1675. | 4-[(3-{4-[(1-ethylpiperidin-4-yl)amino]-1-(2,2,2-trifluoroethyl)-1H-indol-2-yl}prop-2-yn- |
| | 1-yl)amino]-3-methoxybenzene-1-sulfonamide |
| 1676. | 3-methoxy-N-methyl-4-{[3-(4-{[1-(propan-2-yl)piperidin-4-yl]amino}-1-(2,2,2- |
| | trifluoroethyl)-1H-indol-2-yl)prop-2-yn-1-yl]amino}benzamide |
| 1677. | 2-(5-methanesulfonyl-2-{[3-(4-{[1-(propan-2-yl)piperidin-4-yl]amino}-1-(2,2,2- |
| | trifluoroethyl)-1H-indol-2-yl)prop-2-yn-1-yl]amino}phenoxy)acetonitrile |
| 1678. | 2-(3-{[2-(fluoromethoxy)-4-methanesulfonylphenyl]amino}prop-1-yn-1-yl)-N-[1- |
| | (propan-2-yl)piperidin-4-yl]-1-(2,2,2-trifluoroethyl)-1H-indol-4-amine |
| 1679. | 2-{3-[(4-methanesulfonyl-2-methoxyphenyl)amino]prop-1-yn-1-yl}-N-[1-(propan-2- |
| | yl)piperidin-4-yl]-1-(2,2,2-trifluoroethyl)-1H-indol-4-amine |
| 1680. | 3-methoxy-4-{[3-(4-{[1-(propan-2-yl)piperidin-4-yl]amino}-1-(2,2,2-trifluoroethyl)-1H- |
| | indol-2-yl)prop-2-yn-1-yl]amino}benzene-1-sulfonamide |
| 1681. | 2-[2-(2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethoxy)ethoxy]ethan-1-ol |
| 1682. | 4-({4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}methyl)-1,3-dioxolan-2-one |
| 1683. | 3-methoxy-4-({3-[4-({1-[(2-oxo-1,3-dioxolan-4-yl)methyl]piperidin-4-yl}amino)-1- |
| | (2,2,2-trifluoroethyl)-1H-indol-2-yl]prop-2-yn-1-yl}amino)benzene-1-sulfonamide |
| 1684. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(1,2-oxazol-3-yl)benzene-1-sulfonamide |
| 1685. | 4-((3-(4-((1-(2,3-dihydroxypropyl)piperidin-4-yl)amino)-1-(2,2,2-trifluoroethyl)-1H- |
| | indol-2-yl)prop-2-yn-1-yl)amino)-3-methoxybenzenesulfonamide |
| 1686. | 4-({3-[4-({1-[(2S)-2,3-dihydroxypropyl]piperidin-4-yl}amino)-1-(2,2,2-trifluoroethyl)- |
| | 1H-indol-2-yl]prop-2-yn-1-yl}amino)-3-methoxybenzene-1-sulfonamide |
| 1687. | 4-({3-[4-({1-[(2R)-2,3-dihydroxypropyl]piperidin-4-yl}amino)-1-(2,2,2-trifluoroethyl)- |
| | 1H-indol-2-yl]prop-2-yn-1-yl}amino)-3-methoxybenzene-1-sulfonamide |
| 1688. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(5-methyl-1,2-oxazol-3-yl)benzene-1- |
| | sulfonamide |
| 1689. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(oxan-4-yl)benzene-1-sulfonamide |
| 1690. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-N-(2-hydroxyethyl)-3-methoxy-N-methylbenzene-1- |
| | sulfonamide |
| 1691. | 2-(5-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-4-methoxypyridin-2-yl)-2-methylpropanenitrile |
| 1692. | N-(2-hydroxyethyl)-3-methoxy-N-methyl-4-{[3-(4-{[1-(oxan-4-yl)piperidin-4-yl]amino}- |
| | 1-(2,2,2-trifluoroethyl)-1H-indol-2-yl)prop-2-yn-1-yl]amino}benzene-1-sulfonamide |
| 1693. | 3-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)propane-1,2-diol |
| 1694. | (2R)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propane-1,2-diol |
| 1695. | (2S)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propane-1,2-diol |
| 1696. | 3-[4-({2-[3-({2-methoxy-4-[(4-methylpiperazin-1-yl)sulfonyl]phenyl}amino)prop-1-yn-1- |
| | yl]-1-(2,2,2-trifluoroethyl)-1H-indol-4-yl}amino)piperidin-1-yl]propane-1,2-diol |
| 1697. | 4-((3-(4-((1-(2,3-dihydroxypropyl)piperidin-4-yl)amino)-1-(2,2,2-trifluoroethyl)-1H- |
| | indol-2-yl)prop-2-yn-1-yl)amino)-3-methoxybenzoic acid |
| 1698. | methyl 4-((3-(4-((1-(2,3-dihydroxypropyl)piperidin-4-yl)amino)-1-(2,2,2-trifluoroethyl)- |
| | 1H-indol-2-yl)prop-2-yn-1-yl)amino)-3-methoxybenzoate |
| 1699. | 3-methoxy-4-[(3-{4-[(1-{[(4R)-2-oxo-1,3-dioxolan-4-yl]methyl}piperidin-4-yl)amino]-1- |
| | (2,2,2-trifluoroethyl)-1H-indol-2-yl}prop-2-yn-1-yl)amino]benzene-1-sulfonamide |
| 1700. | (4R)-4-({4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}methyl)-1,3-dioxolan-2-one |
| 1701. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N,N-dimethylbenzene-1-sulfonamide |
| 1702. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}EtOAc |
| 1703. | N-(1-{[(4R)-2,2-dimethyl-1,3-dioxolan-4-yl]methyl}piperidin-4-yl)-2-{3-[(4- |
| | methanesulfonyl-2-methoxyphenyl)amino]prop-1-yn-1-yl}-1-(2,2,2-trifluoroethyl)-1H- |
| | indol-4-amine |
| 1704. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-yl acetate |
| 1705. | 4-[(3-{4-[(1-{[(4R)-2,2-dimethyl-1,3-dioxolan-4-yl]methyl}piperidin-4-yl)amino]-1- |
| | (2,2,2-trifluoroethyl)-1H-indol-2-yl}prop-2-yn-1-yl)amino]-3-methoxybenzene-1- |
| | sulfonamide |
| 1706. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1707. | 1-(4-((2-(3-((4-(ethylsulfonyl)-2-methoxyphenyl)amino)prop-1-yn-1-yl)-1-(2,2,2- |
| | trifluoroethyl)-1H-indol-4-yl)amino)piperidin-1-yl)-3-methoxypropan-2-ol |
| 1708. | 1-ethoxy-3-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)propan-2-ol |
| 1709. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(2-methoxyethyl)-N-methylbenzene-1- |
| | sulfonamide |
| 1710. | 1-(acetyloxy)-3-{4-[(2-{3-[(2-methoxy-4-sulfamoylphenyl)amino]prop-1-yn-1-yl}-1- |
| | (2,2,2-trifluoroethyl)-1H-indol-4-yl)amino]piperidin-1-yl}propan-2-yl acetate |
| 1711. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxybenzene-1-sulfonamide |
| 1712. | 3-methoxy-4-{[3-(4-{[1-(2-methoxyacetyl)piperidin-4-yl]amino}-1-(2,2,2-trifluoroethyl)- |
| | 1H-indol-2-yl)prop-2-yn-1-yl]amino}benzene-1-sulfonamide |
| 1713. | 1-(4-(N-(2-(3-((2-methoxy-4-(methylsulfonyl)phenyl)amino)prop-1-yn-1-yl)-1-(2,2,2- |
| | trifluoroethyl)-1H-indol-4-yl)acetamido)piperidin-1-yl)propan-2-yl acetate |
| 1714. | 1-[4-(4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxybenzenesulfonyl)piperazin-1-yl]ethan-1-one |
| 1715. | (4S)-4-({4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}methyl)-1,3-dioxolan-2-one |
| 1716. | 1-(acetyloxy)-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}propan-2-yl acetate |
| 1717. | N-[1-(2,3-dimethoxypropyl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1718. | 4-{[3-(4-{[1-(2,3-dimethoxypropyl)piperidin-4-yl]amino}-1-(2,2,2-trifluoroethyl)-1H- |
| | indol-2-yl)prop-2-yn-1-yl]amino}-3-methoxybenzene-1-sulfonamide |
| 1719. | 3-(4-{[2-(3-{[2-methoxy-4-(morpholine-4-sulfonyl)phenyl]amino}prop-1-yn-1-yl)-1- |
| | (2,2,2-trifluoroethyl)-1H-indol-4-yl]amino}piperidin-1-yl)propane-1,2-diol |
| 1720. | 4-({3-[4-({1-[(2R)-2,3-dihydroxypropyl]piperidin-4-yl}amino)-1-(2,2,2-trifluoroethyl)- |
| | 1H-indol-2-yl]prop-2-yn-1-yl}amino)-3-methoxybenzamide |
| 1721. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethyl 2-methylpropanoate |
| 1722. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethyl propanoate |
| 1723. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-(propanoyloxy)propan-2-yl |
| | propanoate |
| 1724. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}-3-(propanoyloxy)propan-2-yl |
| | propanoate |
| 1725. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-[(2-methylpropanoyl)oxy]propan- |
| | 2-yl 2-methylpropanoate |
| 1726. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}-3-[(2-methylpropanoyl)oxy]propan- |
| | 2-yl 2-methylpropanoate |
| 1727. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-yl propanoate |
| 1728. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-yl 2-methylpropanoate |
| 1729. | 2-hydroxy-3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propyl 2-methylpropanoate |
| 1730. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl acetate |
| 1731. | 2-hydroxy-3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propyl propanoate |
| 1732. | N,N-bis(2-hydroxyethyl)-4-{[3-(4-{[1-(2-hydroxypropyl)piperidin-4-yl]amino}-1-(2,2,2- |
| | trifluoroethyl)-1H-indol-2-yl)prop-2-yn-1-yl]amino}-3-methoxybenzene-1-sulfonamide |
| 1733. | 4-{[3-(4-{[1-(2-hydroxypropyl)piperidin-4-yl]amino}-1-(2,2,2-trifluoroethyl)-1H-indol-2- |
| | yl)prop-2-yn-1-yl]amino}-3-methoxy-N-methylbenzamide |
| 1734. | (S)-4-((3-(4-((1-(2-hydroxy-3-methoxypropyl)piperidin-4-yl)amino)-1-(2,2,2- |
| | trifluoroethyl)-1H-indol-2-yl)prop-2-yn-1-yl)amino)-3-methoxy-N-methylbenzamide |
| 1735. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl 2-methylpropanoate |
| 1736. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl acetate |
| 1737. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}ethyl 2-methylpropanoate |
| 1738. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N-methylbenzamide |
| 1739. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl propanoate |
| 1740. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl 2- |
| | methylpropanoate |
| 1741. | 1-methoxy-3-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl 2-methylpropanoate |
| 1742. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl propanoate |
| 1743. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3- |
| | methoxybenzenesulfonyl)acetamide |
| 1744. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3- |
| | methoxybenzenesulfonyl)propanamide |
| 1745. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}ethyl propanoate |
| 1746. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N-methylbenzene-1-sulfonamide |
| 1747. | 1-methoxy-3-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl propanoate |
| 1748. | (2R)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1749. | (2S)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1750. | 3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1751. | 3-methoxy-4-((3-(4-((1-(2-methoxyethyl)piperidin-4-yl)amino)-1-(2,2,2-trifluoroethyl)- |
| | 1H-indol-2-yl)prop-2-yn-1-yl)amino)benzenesulfonamide |
| 1752. | 2-(3-((2-methoxy-4-(methylsulfonyl)phenyl)amino)prop-1-yn-1-yl)-N-(1-(2- |
| | methoxyethyl)piperidin-4-yl)-1-(2,2,2-trifluoroethyl)-1H-indol-4-amine |
| 1753. | 3-methoxy-4-{[3-(4-{[1-(2-methoxyethyl)piperidin-4-yl]amino}-1-(2,2,2-trifluoroethyl)- |
| | 1H-indol-2-yl)prop-2-yn-1-yl]amino}-N-methylbenzamide |
| 1754. | 3-methoxy-4-{[3-(4-{[1-(2-methoxyethyl)piperidin-4-yl]amino}-1-(2,2,2-trifluoroethyl)- |
| | 1H-indol-2-yl)prop-2-yn-1-yl]amino}-N-methylbenzene-1-sulfonamide |
| 1755. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}EtOAc |
| 1756. | 1-methoxy-3-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl acetate |
| 1757. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxybenzamide |
| 1758. | 2-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-5-methanesulfonylphenol |
| 1759. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N,N-dimethylbenzene-1-sulfonamide |
| 1760. | 4-({3-[4-({1-[(2R)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxybenzene-1-sulfonamide |
| 1761. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzenesulfonyl)-N- |
| | methylpropanamide |
| 1762. | 1-(4-{[2-(3-{[2-(2-fluoroethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1763. | 1-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)tetradecan-1-one |
| 1764. | 1-methoxy-3-(4-{[2-(3-{[2-methoxy-4-(propanamidosulfonyl)phenyl]amino}prop-1-yn-1- |
| | yl)-1-(2,2,2-trifluoroethyl)-1H-indol-4-yl]amino}piperidin-1-yl)propan-2-yl propanoate |
| 1765. | 1-(4-{[2-(3-{[2-methoxy-4-(propanamidosulfonyl)phenyl]amino}prop-1-yn-1-yl)-1- |
| | (2,2,2-trifluoroethyl)-1H-indol-4-yl]amino}piperidin-1-yl)-3-(propanoyloxy)propan-2-yl |
| | propanoate |
| 1766. | (2R)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methoxypropan-1-ol |
| 1767. | (2S)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methoxypropan-1-ol |
| 1768. | 1-{4-[(2-{3-[(2-ethoxy-4-methanesulfonylphenyl)amino]prop-1-yn-1-yl}-1-(2,2,2- |
| | trifluoroethyl)-1H-indol-4-yl)amino]piperidin-1-yl}-3-methoxypropan-2-ol |
| 1769. | 4-({3-[4-({1-[(2S)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxybenzene-1-sulfonamide |
| 1770. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N-(propan-2-yl)benzamide |
| 1771. | 3-hydroxy-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}benzene-1-sulfonamide |
| 1772. | (2R)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl 2- |
| | methylpropanoate |
| 1773. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(N- |
| | propionylsulfamoyl)phenyl)propionamide |
| 1774. | 1-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)-3-methoxypropan-2-ol |
| 1775. | (2S)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl 2- |
| | methylpropanoate |
| 1776. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(methylsulfonyl)phenyl)acetamide |
| 1777. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(methylsulfonyl)phenyl)propionamide |
| 1778. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(methylsulfonyl)phenyl)isobutyramide |
| 1779. | 2-(2-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5- |
| | methanesulfonylphenoxy)acetonitrile |
| 1780. | 1-{4-[(2-{3-[(4-methanesulfonyl-2-methoxyphenyl)amino]prop-1-yn-1-yl}-5-methoxy-1- |
| | (2,2,2-trifluoroethyl)-1H-indol-4-yl)amino]piperidin-1-yl}-3-methoxypropan-2-ol |
| 1781. | 1-(4-{[2-(3-{[2-(2-hydroxyethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1782. | 1-(4-{[2-(3-{[4-methanesulfonyl-2-(2,2,2-trifluoroethoxy)phenyl]amino}prop-1-yn-1-yl)- |
| | 1-(2,2,2-trifluoroethyl)-1H-indol-4-yl]amino}piperidin-1-yl)-3-methoxypropan-2-ol |
| 1783. | 1-[4-({2-[3-(2-amino-4-methanesulfonylphenoxy)prop-1-yn-1-yl]-1-(2,2,2-trifluoroethyl)- |
| | 1H-indol-4-yl}amino)piperidin-1-yl]-3-methoxypropan-2-ol |
| 1784. | 4-({3-[4-({1-[(2R)-2-hydroxy-3-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1785. | 4-({3-[4-({1-[(2S)-2-hydroxy-3-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1786. | 4-({3-[4-({1-[(2R)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1787. | 4-({3-[4-({1-[(2S)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1788. | 1-{4-[(2-{3-[4-methanesulfonyl-2-(methylamino)phenoxy]prop-1-yn-1-yl}-1-(2,2,2- |
| | trifluoroethyl)-1H-indol-4-yl)amino]piperidin-1-yl}-3-methoxypropan-2-ol |
| 1789. | 3-(2-fluoroethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1790. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-(2-methoxyethoxy)-N-methylbenzamide |
| 1791. | 3-(cyanomethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1792. | N-ethyl-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzamide |
| 1793. | 1-(4-{[2-(3-{[2-methoxy-4-(methylcarbamoyl)phenyl]amino}prop-1-yn-1-yl)-1-(2,2,2- |
| | trifluoroethyl)-1H-indol-4-yl]amino}piperidin-1-yl)-3-(propanoyloxy)propan-2-yl |
| | propanoate |
| 1794. | 1-methoxy-3-(4-{[2-(3-{[2-methoxy-4-(methylcarbamoyl)phenyl]amino}prop-1-yn-1-yl)- |
| | 1-(2,2,2-trifluoroethyl)-1H-indol-4-yl]amino}piperidin-1-yl)propan-2-yl propanoate |
| 1795. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-methylbenzamide |
| 1796. | 3-(2-cyanoethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1797. | 1-ethoxy-3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-2-ol |
| 1798. | 2-(2-{[3-(4-{[1-(2-hydroxypropyl)piperidin-4-yl]amino}-1-(2,2,2-trifluoroethyl)-1H- |
| | indol-2-yl)prop-2-yn-1-yl]amino}-5-methanesulfonylphenoxy)acetonitrile |
| 1799. | 1-(4-{[2-(3-{[4-(ethanesulfonyl)-2-methoxyphenyl]amino}prop-1-yn-1-yl)-1-(2,2,2- |
| | trifluoroethyl)-1H-indol-4-yl]amino}piperidin-1-yl)-3-methoxypropan-2-ol |
| 1800. | 3-(fluoromethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1801. | 1-(4-{[2-(3-{[2-(fluoromethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1802. | 2-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxyphenyl)-2- |
| | methylpropanenitrile |
| 1803. | (2S)-1-(4-{[2-(3-{[2-(fluoromethoxy)-4-methanesulfonylphenyl]amino}prop-1-yn-1-yl)- |
| | 1-(2,2,2-trifluoroethyl)-1H-indol-4-yl]amino}piperidin-1-yl)-3-methoxypropan-2-ol |
| 1804. | (2R)-1-(4-{[2-(3-{[2-(fluoromethoxy)-4-methanesulfonylphenyl]amino}prop-1-yn-1-yl)- |
| | 1-(2,2,2-trifluoroethyl)-1H-indol-4-yl]amino}piperidin-1-yl)-3-methoxypropan-2-ol |
| 1805. | 1-(4-{[2-(3-{[2-(difluoromethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1806. | 3-(2-hydroxy-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}propoxy)propane-1,2-diol |
| 1807. | 1-{4-[(2-{3-[(5-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1808. | 3-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-6-(methylsulfonyl)benzo[d]oxazol-2(3H)-one |
| 1809. | 1-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-3-methoxypropan-2-ol |
| 1810. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-(2,2,2-trifluoroethoxy)propan-2-ol |
| 1811. | 4-hydroxy-9-[(2-{3-[(4-methanesulfonyl-2-methoxyphenyl)amino]prop-1-yn-1-yl}-1- |
| | (2,2,2-trifluoroethyl)-1H-indol-4-yl)amino]-2-oxa-6λ⁵-azaspiro[5.5]undecan-6-ylium |
| 1812. | 2-{3-[(4-methanesulfonyl-2-methoxyphenyl)amino]prop-1-yn-1-yl}-N-[1-(3- |
| | methoxypropyl)piperidin-4-yl]-1-(2,2,2-trifluoroethyl)-1H-indol-4-amine |
| 1813. | 1-{4-[(2-{3-[(4-methanesulfonyl-2-methoxyphenyl)amino]prop-1-yn-1-yl}-3-methyl-1- |
| | (2,2,2-trifluoroethyl)-1H-indol-4-yl)amino]piperidin-1-yl}-3-methoxypropan-2-ol |
| 1814. | 1-{4-[(2-{3-[(5-fluoro-4-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-ol |
| 1815. | 2-{3-[(4-methanesulfonyl-2-methoxyphenyl)amino]prop-1-yn-1-yl}-N-[1-(oxetan-3- |
| | yl)piperidin-4-yl]-1-(2,2,2-trifluoroethyl)-1H-indol-4-amine |
| 1816. | 2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1817. | (R)-2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1818. | (S)-2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1819. | 2-(3-((2-methoxy-4-(methylsulfonyl)phenyl)amino)prop-1-yn-1-yl)-N-(1- |
| | ((tetrahydrofuran-2-yl)methyl)piperidin-4-yl)-1-(2,2,2-trifluoroethyl)-1H-indol-4-amine |
| 1820. | 2-fluoro-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5-methoxybenzamide |
| 1821. | 1-methoxy-3-(4-((2-(3-(((3S,4R)-3-methoxytetrahydro-2H-pyran-4-yl)amino)prop-1-yn- |
| | 1-yl)-1-(2,2,2-trifluoroethyl)-1H-indol-4-yl)amino)piperidin-1-yl)propan-2-ol |
| 1822. | 1-(4-{[2-(3-{[4-(cyclopropanesulfonyl)-2-methoxyphenyl]amino}prop-1-yn-1-yl)-1- |
| | (2,2,2-trifluoroethyl)-1H-indol-4-yl]amino}piperidin-1-yl)-3-methoxypropan-2-ol |
| 1823. | 3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propanenitrile |
| 1824. | 4-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)butanenitrile |
| 1825. | 2-{3-[(4-methanesulfonyl-2-methoxyphenyl)amino]prop-1-yn-1-yl}-N-{1-[(oxolan-2- |
| | yl)methyl]piperidin-4-yl}-1-(2,2,2-trifluoroethyl)-1H-indol-4-amine |
| 1826. | 1-methoxy-3-(4-{[2-(3-{[2-methoxy-4-(trifluoromethyl)phenyl]amino}prop-1-yn-1-yl)-1- |
| | (2,2,2-trifluoroethyl)-1H-indol-4-yl]amino}piperidin-1-yl)propan-2-ol |
| 1827. | 2-fluoro-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5-methoxy-N-methylbenzamide |
| 1828. | 1-(4-{[2-(3-{[4-(benzenesulfonyl)-2-methoxyphenyl]amino}prop-1-yn-1-yl)-1-(2,2,2- |
| | trifluoroethyl)-1H-indol-4-yl]amino}piperidin-1-yl)-3-methoxypropan-2-ol |
| 1829. | 2-{3-[(4-methanesulfonyl-2-methoxyphenyl)amino]prop-1-yn-1-yl}-N-[1-(2-methoxy-2- |
| | methylpropyl)piperidin-4-yl]-1-(2,2,2-trifluoroethyl)-1H-indol-4-amine |
| 1830. | 2-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)ethan-1-ol |
| 1831. | 1-methoxy-3-(4-{[2-(3-{[2-methoxy-4-(propane-2-sulfonyl)phenyl]amino}prop-1-yn-1- |
| | yl)-1-(2,2,2-trifluoroethyl)-1H-indol-4-yl]amino}piperidin-1-yl)propan-2-ol |
| 1832. | 1-{3-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1833. | 1-methoxy-3-(4-((2-(3-(((3R,4R)-3-methoxytetrahydro-2H-pyran-4-yl)amino)prop-1-yn- |
| | 1-yl)-1-(2,2,2-trifluoroethyl)-1H-indol-4-yl)amino)piperidin-1-yl)propan-2-ol |
| 1834. | 1-{4-[(2-{3-[(4-methanesulfonyl-2-methoxyphenyl)amino]prop-1-yn-1-yl}-1-propyl-1H- |
| | indol-4-yl)amino]piperidin-1-yl}-3-methoxypropan-2-ol |
| 1835. | 1-(4-{[1-(2-fluoroethyl)-2-{3-[(4-methanesulfonyl-2-methoxyphenyl)amino]prop-1-yn-1- |
| | yl}-1H-indol-4-yl]amino}piperidin-1-yl)-3-methoxypropan-2-ol |
| 1836. | 2-(4-{[1-(2-hydroxy-3-methoxypropyl)piperidin-4-yl]amino}-2-{3-[(4-methanesulfonyl- |
| | 2-methoxyphenyl)amino]prop-1-yn-1-yl}-1H-indol-1-yl)acetonitrile |
| 1837. | 1-(4-{[1-(2-chloroethyl)-2-{3-[(4-methanesulfonyl-2-methoxyphenyl)amino]prop-1-yn-1- |
| | yl}-1H-indol-4-yl]amino}piperidin-1-yl)-3-methoxypropan-2-ol |
| 1838. | rac-1-[(3R,4S)-4-[(2-{3-[(4-methanesulfonyl-2-methoxyphenyl)amino]prop-1-yn-1-yl}-1- |
| | (2,2,2-trifluoroethyl)-1H-indol-4-yl)amino]-3-methylpiperidin-1-yl]-3-methoxypropan-2-ol |
| 1839. | rac-1-[(3R,4R)-4-[(2-{3-[(4-methanesulfonyl-2-methoxyphenyl)amino]prop-1-yn-1-yl}- |
| | 1-(2,2,2-trifluoroethyl)-1H-indol-4-yl)amino]-3-methylpiperidin-1-yl]-3-methoxypropan- |
| | 2-ol |
| 1840. | 1-{4-[(2-{3-[(4-methanesulfonyl-2-methylphenyl)amino]prop-1-yn-1-yl}-1-(2,2,2- |
| | trifluoroethyl)-1H-indol-4-yl)amino]piperidin-1-yl}-3-methoxypropan-2-ol |
| 1841. | 1-[4-({1-[(2,2-difluorocyclopropyl)methyl]-2-{3-[(4-methanesulfonyl-2- |
| | methoxyphenyl)amino]prop-1-yn-1-yl}-1H-indol-4-yl}amino)piperidin-1-yl]-3- |
| | methoxypropan-2-ol |
| 1842. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxybenzoic acid |
| 1843. | 2-{3-[(4-methanesulfonyl-2-methoxyphenyl)amino]prop-1-yn-1-yl}-N-[1-(3- |
| | methanesulfonylpropyl)piperidin-4-yl]-1-(2,2,2-trifluoroethyl)-1H-indol-4-amine |
| 1844. | 4-{[3-(4-{[1-(3-methanesulfonylpropyl)piperidin-4-yl]amino}-1-(2,2,2-trifluoroethyl)- |
| | 1H-indol-2-yl)prop-2-yn-1-yl]amino}-3-methoxybenzoic acid |
| 1845. | 1-(4-{[2-(3-{[4-methanesulfonyl-2-(trifluoromethyl)phenyl]amino}prop-1-yn-1-yl)-1- |
| | (2,2,2-trifluoroethyl)-1H-indol-4-yl]amino}piperidin-1-yl)-3-methoxypropan-2-ol |
| 1846. | 1-(4-{[1-(2,2-difluoroethyl)-2-{3-[(4-methanesulfonyl-2-methoxyphenyl)ino]prop-1-yn- |
| | 1-yl}-1H-indol-4-yl]amino}piperidin-1-yl)-3-methoxypropan-2-ol |
| 1847. | 1-(4-{[2-(3-{[4-methanesulfonyl-2-(trifluoromethoxy)phenyl]amino}prop-1-yn-1-yl)-1- |
| | (2,2,2-trifluoroethyl)-1H-indol-4-yl]amino}piperidin-1-yl)-3-methoxypropan-2-ol |
| 1848. | 1-{4-[(2-{3-[(4-methanesulfonyl-2-methoxyphenyl)amino]prop-1-yn-1-yl}-1-(3,3,3- |
| | trifluoropropyl)-1H-indol-4-yl)amino]piperidin-1-yl}-3-methoxypropan-2-ol |
| 1849. | 1-(4-{[1-(2,2-difluoropropyl)-2-{3-[(4-methanesulfonyl-2-methoxyphenyl)amino]prop-1- |
| | yn-1-yl}-1H-indol-4-yl]amino}piperidin-1-yl)-3-methoxypropan-2-ol |
| 1850. | 1-{4-[(2-{3-[(2-chloro-4-methanesulfonylphenyl)amino]prop-1-yn-1-yl}-1-(2,2,2- |
| | trifluoroethyl)-1H-indol-4-yl)amino]piperidin-1-yl}-3-methoxypropan-2-ol |
| 1851. | 1-{4-[(2-{3-[(2-fluoro-4-methanesulfonyl-6-methoxyphenyl)amino]prop-1-yn-1-yl}-1- |
| | (2,2,2-trifluoroethyl)-1H-indol-4-yl)amino]piperidin-1-yl}-3-methoxypropan-2-ol |
| 1852. | 1-(4-((2-(3-((4-((6-oxa-3-azabicyclo[3.1.1]heptan-3-yl)sulfonyl)-2- |
| | methoxyphenyl)amino)prop-1-yn-1-yl)-1-(2,2,2-trifluoroethyl)-1H-indol-4- |
| | yl)amino)piperidin-1-yl)-3-methoxypropan-2-ol |
| 1853. | (2S)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl propanoate |
| 1854. | 1-{4-[(2-{3-[(4-chloro-2-methoxyphenyl)amino]prop-1-yn-1-yl}-1-(2,2,2-trifluoroethyl)- |
| | 1H-indol-4-yl)amino]piperidin-1-yl}-3-methoxypropan-2-ol |
| 1855. | 2-(2-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)ethoxy)ethan-1-ol |
| 1856. | 4-((3-(4-((1-(2,3-dihydroxypropyl)piperidin-4-yl)amino)-1-(2,2,2-trifluoroethyl)-1H- |
| | indol-2-yl)prop-2-yn-1-yl)amino)-N-(2-hydroxyethyl)-3-methoxybenzenesulfonamide |
| 1857. | 2-{3-[(4-methanesulfonyl-2-methoxyphenyl)amino]prop-1-yn-1-yl}-N-[1-(4-methyl-1,3- |
| | thiazol-2-yl)piperidin-4-yl]-1-(2,2,2-trifluoroethyl)-1H-indol-4-amine |
| 1858. | N-(1-cyclopropylpiperidin-4-yl)-2-{3-[(4-methanesulfonyl-2- |
| | methoxyphenyl)amino]prop-1-yn-1-yl}-1-(2,2,2-trifluoroethyl)-1H-indol-4-amine |
| 1859. | 4-[(3-{4-[(1-cyclopropylpiperidin-4-yl)amino]-1-(2,2,2-trifluoroethyl)-1H-indol-2- |
| | yl}prop-2-yn-1-yl)amino]-3-methoxybenzoic acid |
| 1860. | 2-{3-[(4-methanesulfonyl-2-methoxyphenyl)amino]prop-1-yn-1-yl}-N-{1-[(3R)-oxolan- |
| | 3-yl]piperidin-4-yl}-1-(2,2,2-trifluoroethyl)-1H-indol-4-amine |
| 1861. | 2-{3-[(4-methanesulfonyl-2-methoxyphenyl)amino]prop-1-yn-1-yl}-N-{1-[(3S)-oxolan- |
| | 3-yl]piperidin-4-yl}-1-(2,2,2-trifluoroethyl)-1H-indol-4-amine |
| 1862. | 3-methoxy-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzoic acid |
| 1863. | 3-methoxy-4-((3-(4-((1-(tetrahydro-2H-pyran-4-yl)piperidin-4-yl)amino)-1-(2,2,2- |
| | trifluoroethyl)-1H-indol-2-yl)prop-2-yn-1-yl)amino)benzamide |
| 1864. | N-(2-hydroxyethyl)-3-methoxy-4-((3-(4-((1-(tetrahydro-2H-pyran-4-yl)piperidin-4- |
| | yl)amino)-1-(2,2,2-trifluoroethyl)-1H-indol-2-yl)prop-2-yn-1- |
| | yl)amino)benzenesulfonamide |
| 1865. | 3-methoxy-4-((3-(4-((1′-methyl-[1,4′-bipiperidin]-4-yl)amino)-1-(2,2,2-trifluoroethyl)- |
| | 1H-indol-2-yl)prop-2-yn-1-yl)amino)benzamide |
| 1866. | 3-methoxy-4-((3-(4-((1-(2-(4-methylpiperazin-1-yl)-2-oxoethyl)piperidin-4-yl)amino)-1- |
| | (2,2,2-trifluoroethyl)-1H-indol-2-yl)prop-2-yn-1-yl)amino)benzamide |
| 1867. | 2-{2-[(3-{4-[(1-acetylpiperidin-4-yl)amino]-1-(2,2,2-trifluoroethyl)-1H-indol-2-yl}prop- |
| | 2-yn-1-yl)amino]-5-methanesulfonylphenoxy}acetonitrile |
| 1868. | 4-{[3-(4-{[1-(2-hydroxyacetyl)piperidin-4-yl]amino}-1-(2,2,2-trifluoroethyl)-1H-indol-2- |
| | yl)prop-2-yn-1-yl]amino}-3-methoxy-N,N-dimethylbenzene-1-sulfonamide |
| 1869. | 2-hydroxy-1-{4-[(2-{3-[(2-hydroxy-4-methanesulfonylphenyl)amino]prop-1-yn-1-yl}-1- |
| | (2,2,2-trifluoroethyl)-1H-indol-4-yl)amino]piperidin-1-yl}ethan-1-one |
| 1870. | 2-hydroxy-1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethan-1-one |
| 1871. | N-((3S,4S)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1872. | N-((3R,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1873. | N-((3R,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1874. | N-((3R,4S)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1875. | N-((3S,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1876. | 1-(4-((2-(3-((2-methoxy-4-(methylsulfonyl)phenyl)amino)prop-1-yn-1-yl)-1-(2,2,2- |
| | trifluoroethyl)-1H-indol-4-yl)amino)-2-methylpiperidin-1-yl)ethan-1-one |
| 1877. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]-2-methylpiperidin-1-yl}ethan-1-one |
| 1878. | 4-{[3-(4-{[(2S,4S)-1-acetyl-2-methylpiperidin-4-yl]amino}-1-(2,2,2-trifluoroethyl)-1H- |
| | indol-2-yl)prop-2-yn-1-yl]amino}-3-methoxybenzene-1-sulfonamide |
| 1879. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methoxyethan-1-one |
| 1880. | 2-hydroxy-1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-1-one |
| 1881. | 2-methoxy-1-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)ethan-1-one |
| 1882. | 3-methoxy-4-((3-(4-((1-(2-methoxyacetyl)piperidin-4-yl)amino)-1-(2,2,2-trifluoroethyl)- |
| | 1H-indol-2-yl)prop-2-yn-1-yl)amino)benzenesulfonamide |
| 1883. | 4-{[3-(4-{[1-(2-hydroxypropanoyl)piperidin-4-yl]amino}-1-(2,2,2-trifluoroethyl)-1H- |
| | indol-2-yl)prop-2-yn-1-yl]amino}-3-methoxybenzene-1-sulfonamide |
| 1884. | 3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-oxopropanenitrile |
| 1885. | 4-{[3-(4-{[1-(2-cyanoacetyl)piperidin-4-yl]amino}-1-(2,2,2-trifluoroethyl)-1H-indol-2- |
| | yl)prop-2-yn-1-yl]amino}-3-methoxybenzene-1-sulfonamide |
| 1886. | 4-{[3-(4-{[1-(2-hydroxyacetyl)piperidin-4-yl]amino}-1-(2,2,2-trifluoroethyl)-1H-indol-2- |
| | yl)prop-2-yn-1-yl]amino}-3-methoxybenzene-1-sulfonamide |
| 1887. | 4-[(3-{4-[(1-acetylpiperidin-4-yl)amino]-1-(2,2,2-trifluoroethyl)-1H-indol-2-yl}prop-2- |
| | yn-1-yl)amino]-3-methoxybenzene-1-sulfonamide |
| 1888. | 2-(dimethylamino)-1-{4-[(2-{3-[(4-methanesulfonyl-2-methoxyphenyl)amino]prop-1-yn- |
| | 1-yl}-1-(2,2,2-trifluoroethyl)-1H-indol-4-yl)amino]piperidin-1-yl}ethan-1-one |
| 1889. | 4-((3-(4-((1-(dimethylglycyl)piperidin-4-yl)amino)-1-(2,2,2-trifluoroethyl)-1H-indol-2- |
| | yl)prop-2-yn-1-yl)amino)-3-methoxybenzenesulfonamide |
| 1890. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methylpropan-1-one |
| 1891. | 3-methoxy-4-{[3-(4-{[1-(2-methylpropanoyl)piperidin-4-yl]amino}-1-(2,2,2- |
| | trifluoroethyl)-1H-indol-2-yl)prop-2-yn-1-yl]amino}benzene-1-sulfonamide |
| 1892. | 4-((2-(3-((2-methoxy-4-(methylsulfonyl)phenyl)amino)prop-1-yn-1-yl)-1-(2,2,2- |
| | trifluoroethyl)-1H-indol-4-yl)amino)-N,N-dimethylpiperidine-1-carboxamide |
| 1893. | 4-[(3-{4-[(1-acetylpiperidin-4-yl)amino]-1-(2,2,2-trifluoroethyl)-1H-indol-2-yl}prop-2- |
| | yn-1-yl)amino]-3-methoxy-N,N-dimethylbenzene-1-sulfonamide |
| 1894. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-1-one |
| 1895. | 3-methoxy-4-[(3-{4-[(1-propanoylpiperidin-4-yl)amino]-1-(2,2,2-trifluoroethyl)-1H- |
| | indol-2-yl}prop-2-yn-1-yl)amino]benzene-1-sulfonamide |
| 1896. | 1-(4-{[2-(3-{[2-methoxy-4-(morpholine-4-sulfonyl)phenyl]amino}prop-1-yn-1-yl)-1- |
| | (2,2,2-trifluoroethyl)-1H-indol-4-yl]amino}piperidin-1-yl)ethan-1-one |
| 1897. | 3-methoxy-4-[(3-{4-[(1-{[(4S)-2-oxo-1,3-dioxolan-4-yl]methyl}piperidin-4-yl)amino]-1- |
| | (2,2,2-trifluoroethyl)-1H-indol-2-yl}prop-2-yn-1-yl)amino]benzene-1-sulfonamide |
| 1898. | N-((3-methoxy-4-((3-(4-((1-((2-oxo-1,3-dioxolan-4-yl)methyl)piperidin-4-yl)amino)-1- |
| | (2,2,2-trifluoroethyl)-1H-indol-2-yl)prop-2-yn-1-yl)amino)phenyl)sulfonyl)propionamide |
| 1899. | N-[3-methoxy-4-({3-[4-({1-[(2-oxo-1,3-dioxolan-4-yl)methyl]piperidin-4-yl}amino)-1- |
| | (2,2,2-trifluoroethyl)-1H-indol-2-yl]prop-2-yn-1-yl}amino)benzenesulfonyl]acetamide |
| 1900. | 3-methoxy-N-methyl-4-({3-[4-({1-[(2-oxo-1,3-dioxolan-4-yl)methyl]piperidin-4- |
| | yl}amino)-1-(2,2,2-trifluoroethyl)-1H-indol-2-yl]prop-2-yn-1-yl}amino)benzamide |
| 1901. | 2-(3-((2-methoxy-4-(methylsulfonyl)phenyl)amino)prop-1-yn-1-yl)-N- |
| | (octahydroindolizin-7-yl)-1-(2,2,2-trifluoroethyl)-1H-indol-4-amine |
| 1902. | N-[(7R,8aS)-octahydroindolizin-7-yl]-2-{3-[(4-methanesulfonyl-2- |
| | methoxyphfenyl)amino]prop-1-yn-1-yl}-1-(2,2,2-trifluoroethyl)-1H-indol-4-amine |
| 1903. | N-[(7R,8aR)-octahydroindolizin-7-yl]-2-{3-[(4-methanesulfonyl-2- |
| | methoxyphenyl)amino]prop-1-yn-1-yl}-1-(2,2,2-trifluoroethyl)-1H-indol-4-amine |
| 1904. | rac-(3R,4S)-3-[(2-{3-[(4-methanesulfonyl-2-methoxyphenyl)amino]prop-1-yn-1-yl}-1- |
| | (2,2,2-trifluoroethyl)-1H-indol-4-yl)amino]-1-methylpiperidin-4-ol |
| 1905. | rac-(3R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]-1-methylpiperidin-3-ol |
| 1906. | 3-methoxy-4-((3-(4-((1-(tetrahydro-2H-pyran-4-yl)piperidin-4-yl)amino)-1-(2,2,2- |
| | trifluoroethyl)-1H-indol-2-yl)prop-2-yn-1-yl)amino)benzenesulfonamide |
| 1907. | rac-N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1908. | N-((3S,4S)-3-fluoro-1-methylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1909. | N-[(3S,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1910. | N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1911. | N-[(3S,4R)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1912. | N-[(3R,4R)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1913. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzene-1-sulfonamide |
| 1914. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzene-1-sulfonamide |
| 1915. | rac-methyl 4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1916. | rac-methyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1917. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzoic acid |
| 1918. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1919. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1920. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzamide |
| 1921. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-methylbenzamide |
| 1922. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-3-methoxy-N-methylbenzamide |
| 1923. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxy-N-methylbenzamide |
| 1924. | 2-fluoro-4-((3-(4-(((3R,4S)-3-fluoro-1-methylpiperidin-4-yl)amino)-1-(2,2,2- |
| | trifluoroethyl)-1H-indol-2-yl)prop-2-yn-1-yl)amino)-5-methoxy-N-methylbenzamide |
| 1925. | 2-fluoro-4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-5-methoxy-N-methylbenzamide |
| 1926. | 4-{[3-(4-{[(3R,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1927. | 4-{[3-(4-{[(3S,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1928. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzene-1-sulfonamide |
| 1929. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzene-1-sulfonamide |
| 1930. | methyl 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1931. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzamide |
| 1932. | methyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1933. | rac-N-[(3R,4S)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1934. | rac-4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1935. | rac-ethyl 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1936. | rac-ethyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1937. | (2R)-1-(acetyloxy)-3-[(3RS,4SR)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 1938. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzamide |
| 1939. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzamide |
| 1940. | N-[(3S,4R)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1941. | N-[(3R,4S)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1942. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-methylbenzamide |
| 1943. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxy-N-methylbenzamide |
| 1944. | rac-2-hydroxypropyl 4-{[3-(4-{[(3R,4S)-3-fluoro-1-methylpiperidin-4-yl]amino}-1- |
| | (2,2,2-trifluoroethyl)-1H-indol-2-yl)prop-2-yn-1-yl]amino}-3-methoxybenzoate |
| 1945. | 4-((3-(4-(((3R,4S)-3-fluoro-1-methylpiperidin-4-yl)amino)-1-(2,2,2-trifluoroethyl)-1H- |
| | indol-2-yl)prop-2-yn-1-yl)amino)-N-isopropyl-3-methoxybenzamide |
| 1946. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(propan-2-yl)benzamide |
| 1947. | 4-{[3-(4-{[(3S,4R)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1948. | 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1949. | rac-2-[(3R,4S)-3-fluoro-4-[(2-{3-[(4-methanesulfonyl-2-methoxyphenyl)amino]prop-1- |
| | yn-1-yl}-1-(2,2,2-trifluoroethyl)-1H-indol-4-yl)amino]piperidin-1-yl]acetamide |
| 1950. | N-[(3S,4R)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1951. | N-[(3R,4S)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1952. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-(2-methoxyethyl)benzamide |
| 1953. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N,N-dimethylbenzamide |
| 1954. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-N-(2-hydroxy-3-methoxypropyl)-3- |
| | methoxybenzamide |
| 1955. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-(propan-2-yl)benzamide |
| 1956. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-(oxan-4-yl)benzamide |
| 1957. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(oxan-4-yl)benzamide |
| 1958. | rac-N-[(3R,4S)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1959. | ethyl 4-{[3-(4-{[(3S,4R)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1960. | ethyl 4-((3-(4-(((3R,4S)-1-ethyl-3-fluoropiperidin-4-yl)amino)-1-(2,2,2-trifluoroethyl)- |
| | 1H-indol-2-yl)prop-2-yn-1-yl)amino)-3-methoxybenzoate |
| 1961. | ethyl 4-{[3-(4-{[(3S,4R)-1-ethyl-3-fluoropiperidin-4-yl]amino}-1-(2,2,2-trifluoroethyl)- |
| | 1H-indol-2-yl)prop-2-yn-1-yl]amino}-3-methoxybenzoate |
| 1962. | ethyl 4-{[3-(4-{[(3R,4S)-1-ethyl-3-fluoropiperidin-4-yl]amino}-1-(2,2,2-trifluoroethyl)- |
| | 1H-indol-2-yl)prop-2-yn-1-yl]amino}-3-methoxybenzoate |
| 1963. | ethyl 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1964. | 2-fluoro-4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-5-methoxy-N-methylbenzamide |
| 1965. | 2-fluoro-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-5-methoxy-N-methylbenzamide |
| 1966. | 4-{[3-(4-{[(3R,4S)-1-ethyl-3-fluoropiperidin-4-yl]amino}-1-(2,2,2-trifluoroethyl)-1H- |
| | indol-2-yl)prop-2-yn-1-yl]amino}-3-methoxybenzoic acid |
| 1967. | 4-{[3-(4-{[(3S,4R)-1-ethyl-3-fluoropiperidin-4-yl]amino}-1-(2,2,2-trifluoroethyl)-1H- |
| | indol-2-yl)prop-2-yn-1-yl]amino}-3-methoxybenzoic acid |
| 1968. | rac-N-(2-{bis[(pyridin-2-yl)methyl]amino}ethyl)-4-{[3-(4-{[(3R,4S)-3-fluoro-1- |
| | methylpiperidin-4-yl]amino}-1-(2,2,2-trifluoroethyl)-1H-indol-2-yl)prop-2-yn-1- |
| | yl]amino}-3-methoxybenzamide |
| 1969. | rac-N-[(3R,4S)-3-fluoro-1-methylpiperidin-4-yl]-2-{3-[(4-methanesulfonyl-2- |
| | methoxyphenyl)amino]prop-1-yn-1-yl}-N-methyl-1-(2,2,2-trifluoroethyl)-1H-indol-4- |
| | amine |
| 1970. | rac-4-((3-(4-(((3R,4S)-3-fluoro-1-methylpiperidin-4-yl)amino)-1-(2,2,2-trifluoroethyl)- |
| | 1H-indol-2-yl)prop-2-yn-1-yl)amino)-3-methoxybenzoic acid |
| 1971. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzoic acid |
| 1972. | 2-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-5-methanesulfonylphenol |
| 1973. | 2-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-5-methanesulfonylphenol |
| 1974. | rac-6-fluoro-N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1975. | N-(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzenesulfonyl)acetamide |
| 1976. | N-(4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzenesulfonyl)acetamide |
| 1977. | ethyl 4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzoate |
| 1978. | ethyl 4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzoate |
| 1979. | N-[(3R,4S)-3-fluoro-1-methylpiperidin-4-yl]-2-(3-{[2-(fluoromethoxy)-4- |
| | methanesulfonylphenyl]amino}prop-1-yn-1-yl)-1-(2,2,2-trifluoroethyl)-1H-indol-4-amine |
| 1980. | N-[(3S,4R)-3-fluoro-1-methylpiperidin-4-yl]-2-(3-{[2-(fluoromethoxy)-4- |
| | methanesulfonylphenyl]amino}prop-1-yn-1-yl)-1-(2,2,2-trifluoroethyl)-1H-indol-4-amine |
| 1981. | 2-{3-[(2-ethoxy-4-methanesulfonylphenyl)amino]prop-1-yn-1-yl}-N-[(3S,4R)-3-fluoro-1- |
| | methylpiperidin-4-yl]-1-(2,2,2-trifluoroethyl)-1H-indol-4-amine |
| 1982. | 2-{3-[(2-ethoxy-4-methanesulfonylphenyl)amino]prop-1-yn-1-yl}-N-[(3R,4S)-3-fluoro-1- |
| | methylpiperidin-4-yl]-1-(2,2,2-trifluoroethyl)-1H-indol-4-amine |
| 1983. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxybenzene-1-sulfonamide |
| 1984. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxybenzene-1-sulfonamide |
| 1985. | N-((3S,4R)-3-fluoropiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1986. | N-((3R,4S)-3-fluoropiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1987. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxybenzamide |
| 1988. | 4-((3-(4-(((3R,4S)-3-fluoropiperidin-4-yl)amino)-1-(2,2,2-trifluoroethyl)-1H-indol-2- |
| | yl)prop-2-yn-1-yl)amino)-3-methoxybenzamide |
| 1989. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxy-N-methylbenzamide |
| 1990. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxybenzoic acid |
| 1991. | 2-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-ol |
| 1992. | 2-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-ol |
| 1993. | 2-(dimethylamino)-1-[(3S,4R)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 1994. | 2-(dimethylamino)-1-[(3R,4S)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 1995. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxy-N-methylbenzamide |
| 1996. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxybenzoic acid |
| 1997. | N-[(3S,4R)-3-fluoro-1-(2-methoxyethyl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1998. | N-[(3R,4S)-3-fluoro-1-(2-methoxyethyl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1999. | 4-{[3-(4-{[(3S,4R)-3-fluoro-1-[(2R)-2-hydroxy-3-methoxypropyl]piperidin-4-yl]amino}- |
| | 1-(2,2,2-trifluoroethyl)-1H-indol-2-yl)prop-2-yn-1-yl]amino}-3-methoxybenzene-1- |
| | sulfonamide |
| 2000. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-one |
| 2001. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-one |
| 2002. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-1-one |
| 2003. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-1-one |
| 2004. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]-2-methoxyethan-1-one |
| 2005. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]-2-methoxyethan-1-one |
| 2006. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxybenzamide |
| 2007. | 4-{[3-(4-{[(3R,4S)-3-fluoro-1-[(2R)-2-hydroxy-3-methoxypropyl]piperidin-4-yl]amino}- |
| | 1-(2,2,2-trifluoroethyl)-1H-indol-2-yl)prop-2-yn-1-yl]amino}-3-methoxybenzene-1- |
| | sulfonamide |
| 2008. | N-[(7S,8R)-7-fluoro-1,4-dioxaspiro[4.5]decan-8-yl]-2-{3-[(4-methanesulfonyl-2- |
| | methoxyphenyl)amino]prop-1-yn-1-yl}-1-(2,2,2-trifluoroethyl)-1H-indol-4-amine |
| 2009. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-2-ol |
| 2010. | 1-((3S,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2011. | 1-((3R,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2012. | 4-((3-(4-(((3S,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2013. | 4-((3-(4-(((3R,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2014. | 4-((3-(4-(((3R,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2015. | 4-((3-(4-(((3S,4R)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2016. | methyl 4-((3-(4-(((3S,4R)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoate |
| 2017. | methyl 4-((3-(4-(((3R,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoate |
| 2018. | (R)-1-((3R,4S)-3-fluoro-4-((2-(3-((2-fluoro-6-methoxy-4- |
| | (methylsulfonyl)phenyl)amino)prop-1-yn-1-yl)-1-propyl-1H-indol-4-yl)amino)piperidin- |
| | 1-yl)-3-methoxypropan-2-ol |
| 2019. | (R)-1-((3R,4S)-4-((1-allyl-2-(3-((2-fluoro-6-methoxy-4- |
| | (methylsulfonyl)phenyl)amino)prop-1-yn-1-yl)-1H-indol-4-yl)amino)-3-fluoropiperidin- |
| | 1-yl)-3-methoxypropan-2-ol |
| 2020. | 4-{[3-(4-{[(3S,4R)-1-[(2R)-2,3-dihydroxypropyl]-3-fluoropiperidin-4-yl]amino}-1-(2,2,2- |
| | trifluoroethyl)-1H-indol-2-yl)prop-2-yn-1-yl]amino}-3-methoxybenzene-1-sulfonamide |
| 2021. | 4-{[3-(4-{[(3R,4S)-1-[(2R)-2,3-dihydroxypropyl]-3-fluoropiperidin-4-yl]amino}-1-(2,2,2- |
| | trifluoroethyl)-1H-indol-2-yl)prop-2-yn-1-yl]amino}-3-methoxybenzene-1-sulfonamide |
| 2022. | (2R)-1-[(3RS,4SR)-3-fluoro-4-[(2-{3-[(4-methanesulfonyl-2-methoxyphenyl)amino]prop- |
| | 1-yn-1-yl}-1-propyl-1H-indol-4-yl)amino]piperidin-1-yl]-3-methoxypropan-2-ol |
| 2023. | 4-((3-(4-(((3S,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2024. | 4-((3-(4-(((3S,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2025. | 4-((3-(4-(((3R,4R)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2026. | (2R)-1-(acetyloxy)-3-[(3R,4S)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 2027. | N-[(2R)-2,3-dihydroxypropyl]-4-{[3-(4-{[(3RS,4SR)-3-fluoro-1-[(2R)-2-hydroxy-3- |
| | methoxypropyl]piperidin-4-yl]amino}-1-(2,2,2-trifluoroethyl)-1H-indol-2-yl)prop-2-yn-1- |
| | yl]amino}-3-methoxybenzene-1-sulfonamide |
| 2028. | (2R)-1-(acetyloxy)-3-[(3S,4R)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 2029. | rac-N-(2-{bis[(pyridin-2-yl)methyl]amino}ethyl)-2-[(3R,4S)-3-fluoro-4-[(2-{3-[(4- |
| | methanesulfonyl-2-methoxyphenyl)amino]prop-1-yn-1-yl}-1-(2,2,2-trifluoroethyl)-1H- |
| | indol-4-yl)amino]piperidin-1-yl]acetamide |
| 2030. | 2-amino-1-[(3R,4S)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 2031. | 2-amino-1-[(3S,4R)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 2032. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-2-ol |
| 2033. | 3-(4-(((3S,4R)-3-fluoro-1-methylpiperidin-4-yl)amino)-1-(2,2,2-trifluoroethyl)-1H-indol- |
| | 2-yl)prop-2-yn-1-ol |
| 2034. | (4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-3-methoxybenzoyl)glycine |
| 2035. | methyl 4-{[3-(4-{[(3S,4R)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoate |
| 2036. | 4-{[3-(4-{[(3S,4R)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoic acid |
| 2037. | methyl 4-{[3-(4-{[(3S,4R)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoate |
| 2038. | methyl 4-{[3-(4-{[(3S,4R)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoate |
| 2039. | methyl 4-{[3-(4-{[(3S,4R)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoate |
| 2040. | methyl 4-{[3-(4-{[(3R,4S)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoate |
| 2041. | methyl 4-{[3-(4-{[(3R,4S)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoate |
| 2042. | 4-{[3-(4-{[(3R,4S)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoic acid |
| 2043. | methyl 4-{[3-(4-{[(3R,4S)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoate |
| 2044. | N-[(3S,4R)-3-fluoro-1-methylpiperidin-4-yl]-2-{3-[(2-methoxy-4-{2-oxa-6- |
| | azaspiro[3.3]heptane-6-carbonyl}phenyl)amino]prop-1-yn-1-yl}-1-(2,2,2-trifluoroethyl)- |
| | 1H-indol-4-amine |
| 2045. | N-[(3S,4R)-3-fluoro-1-methylpiperidin-4-yl]-2-{3-[(2-methoxy-4-{7-oxa-2- |
| | azaspiro[3.5]nonane-2-carbonyl}phenyl)amino]prop-1-yn-1-yl}-1-(2,2,2-trifluoroethyl)- |
| | 1H-indol-4-amine |
| 2046. | 4-{[3-(4-{[(3S,4R)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoic acid |
| 2047. | N-[(3S,4R)-3-fluoro-1-methylpiperidin-4-yl]-2-(3-{[2-methoxy-4-(morpholine-4- |
| | carbonyl)phenyl]amino}prop-1-yn-1-yl)-1-(2,2,2-trifluoroethyl)-1H-indol-4-amine |
| 2048. | N-[(3S,4R)-3-fluoro-1-methylpiperidin-4-yl]-2-(3-{[2-methoxy-4-(4-methylpiperazine-1- |
| | carbonyl)phenyl]amino}prop-1-yn-1-yl)-1-(2,2,2-trifluoroethyl)-1H-indol-4-amine |
| 2049. | 4-{[3-(4-{[(3R,4S)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoic acid |
| 2050. | 4-{[3-(4-{[(3R,4S)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoic acid |
| 2051. | methyl 4-{[3-(4-{[(3S,4R)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoate |
| 2052. | methyl 4-{[3-(4-{[(3R,4S)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoate |
| 2053. | methyl 4-{[3-(4-{[(3R,4S)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoate |
| 2054. | 2-((3R,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)acetamide |
| 2055. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(1,3-thiazol-2-yl)benzamide |
| 2056. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(1-methylpiperidin-4-yl)benzamide |
| 2057. | 1-(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzoyl)piperidin-4-ol |
| 2058. | 4-{[3-(4-{[(3S,4R)-1-[(dimethylcarbamoyl)methyl]-3-fluoropiperidin-4-yl]amino}-1- |
| | (2,2,2-trifluoroethyl)-1H-indol-2-yl)prop-2-yn-1-yl]amino}-3-methoxybenzoic acid |
| 2059. | 4-{[3-(4-{[(3S,4R)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoic acid |
| 2060. | tert-butyl (3S,4R)-4-{[2-(3-{[4-(ethanesulfonyl)-2-methoxyphenyl]amino}prop-1-yn-1- |
| | yl)-1-(2,2,2-trifluoroethyl)-1H-indol-4-yl]amino}-3-fluoropiperidine-1-carboxylate |
| 2061. | 2-(3-((4-(ethylsulfonyl)-2-methoxyphenyl)amino)prop-1-yn-1-yl)-N-((3S,4R)-3- |
| | fluoropiperidin-4-yl)-1-(2,2,2-trifluoroethyl)-1H-indol-4-amine |
| 2062. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-[2-(morpholin-4-yl)ethyl]benzamide |
| 2063. | 4-{[3-(4-{[(3R,4S)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoic acid |
| 2064. | 4-{[3-(4-{[(3S,4R)-1-(carbamoylmethyl)-3-fluoropiperidin-4-yl]amino]-1-(2,2,2- |
| | trifluoroethyl)-1H-indol-2-yl)prop-2-yn-1-yl]amino}-3-methoxybenzoic acid |
| 2065. | 4-{[3-(4-{[(3R,4S)-1-[(dimethylcarbamoyl)methyl]-3-fluoropiperidin-4-yl]amino}-1- |
| | (2,2,2-trifluoroethyl)-1H-indol-2-yl)prop-2-yn-1-yl]amino}-3-methoxybenzoic acid |
| 2066. | tert-butyl (3S,4R)-4-[(2-{3-[(2-ethoxy-4-methanesulfonylphenyl)amino]prop-1-yn-1-yl}- |
| | 1-(2,2,2-trifluoroethyl)-1H-indol-4-yl)amino]-3-fluoropiperidine-1-carboxylate |
| 2067. | (2S)-2-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]pentanedioic acid |
| 2068. | (2S)-4-carbamoyl-2-[(4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3- |
| | methoxyphenyl)formamido]butanoic acid |
| 2069. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzene-1-sulfonic acid |
| 2070. | 1,5-dimethyl (2S)-2-[(4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3- |
| | methoxyphenyl)formamido]pentanedioate |
| 2071. | 2-[3-({4-[4-(dimethylamino)piperidine-1-carbonyl]-2-methoxyphenyl}amino)prop-1-yn- |
| | 1-yl]-N-[(3S,4R)-3-fluoro-1-methylpiperidin-4-yl]-1-(2,2,2-trifluoroethyl)-1H-indol-4- |
| | amine |
| 2072. | 4-{[3-(4-{[(3S,4R)-1-(carboxymethyl)-3-fluoropiperidin-4-yl]amino}-1-(2,2,2- |
| | trifluoroethyl)-1H-indol-2-yl)prop-2-yn-1-yl]amino}-3-methoxybenzoic acid |
| 2073. | 2-(3-{[4-(ethanesulfonyl)-2-methoxyphenyl]amino}prop-1-yn-1-yl)-N-[(3S,4R)-3- |
| | fluoropiperidin-4-yl]-1-(2,2,2-trifluoroethyl)-1H-indol-4-amine |
| 2074. | (1R,2R,4S)-2-fluoro-N¹-(2-{3-[(4-methanesulfonyl-2-methoxyphenyl)amino]prop-1-yn- |
| | 1-yl}-1-(2,2,2-trifluoroethyl)-1H-indol-4-yl)cyclohexane-1,4-diamine |
| 2075. | (1R,2R,4S)-2-fluoro-N¹-(2-{3-[(4-methanesulfonyl-2-methoxyphenyl)amino]prop-1-yn- |
| | 1-yl}-1-(2,2,2-trifluoroethyl)-1H-indol-4-yl)-N⁴-methylcyclohexane-1,4-diamine |
| 2076. | (1S,3R,4R)-3-fluoro-N⁴-(2-{3-[(4-methanesulfonyl-2-methoxyphenyl)amino]prop-1-yn- |
| | 1-yl}-1-(2,2,2-trifluoroethyl)-1H-indol-4-yl)-N¹,N¹-dimethylcyclohexane-1,4-diamine |
| 2077. | 2-(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzenesulfonyl)ethan-1-ol |
| 2078. | N-ethyl-4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzamide |
| 2079. | 2-{3-[(2-ethoxy-4-methanesulfonylphenyl)amino]prop-1-yn-1-yl}-N-[(3S,4R)-3- |
| | fluoropiperidin-4-yl]-1-(2,2,2-trifluoroethyl)-1H-indol-4-amine |
| 2080. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N,N-dimethylbenzamide |
| 2081. | N-ethyl-4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxy-N-methylbenzamide |
| 2082. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-methyl-N-(propan-2-yl)benzamide |
| 2083. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(2-methoxyethyl)benzamide |
| 2084. | N-[2-(diethylamino)ethyl]-4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3-methoxybenzamide |
| 2085. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(2-hydroxyethyl)-3-methoxybenzamide |
| 2086. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-[(2R,3R,4R,5S,6R)-2,4,5-trihydroxy-6- |
| | (hydroxymethyl)oxan-3-yl]benzamide |
| 2087. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-N-(1-hydroxypropan-2-yl)-3-methoxybenzamide |
| 2088. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-[(2R)-1-hydroxypropan-2-yl]-3-methoxybenzamide |
| 2089. | N-[3-(4-{[(3S,4R)-3-fluoro-1-methylpiperidin-4-yl]amino}-1-(2,2,2-trifluoroethyl)-1H- |
| | indol-2-yl)prop-2-yn-1-yl]cyclopropanecarboxamide |
| 2090. | (1R,2R)-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-2-phenylcyclopropane-1-carboxamide |
| 2091. | N-[3-(4-{[(3S,4R)-3-fluoro-1-methylpiperidin-4-yl]amino}-1-(2,2,2-trifluoroethyl)-1H- |
| | indol-2-yl)prop-2-yn-1-yl]-1-methyl-1H-pyrrole-3-carboxamide |
| 2092. | 1-ethyl-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-1H-pyrrole-3-carboxamide |
| 2093. | 1-tert-butyl-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-1H-pyrrole-3-carboxamide |
| 2094. | methyl (2S)-4-carbamoyl-2-[(4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxyphenyl)formamido]butanoate |
| 2095. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-N-((R)-2-hydroxypropyl)-3-methoxybenzamide |
| 2096. | rac-4-{[3-(4-{[(3R,4S)-1-ethyl-3-fluoropiperidin-4-yl]amino}-1-(2,2,2-trifluoroethyl)-1H- |
| | indol-2-yl)prop-2-yn-1-yl]amino}-3-methoxybenzoic acid |
| 2097. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-N-((S)-2-hydroxypropyl)-3-methoxybenzamide |
| 2098. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(2-hydroxy-3-methoxypropyl)-3-methoxybenzamide |
| 2099. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-[(2S)-2-hydroxypropyl]-3-methoxybenzamide |
| 2100. | N-(2,3-dihydroxypropyl)-4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3-methoxybenzamide |
| 2101. | N-[(2R)-2,3-dihydroxypropyl]-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2102. | N-[(2S)-2,3-dihydroxypropyl]-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2103. | N-(1,5-dihydroxypentan-3-yl)-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2104. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(3-hydroxy-2-methoxypropyl)-3-methoxybenzamide |
| 2105. | 1-[(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxyphenyl)formamido]-3-[(2- |
| | methylpropanoyl)oxy]propan-2-yl 2-methylpropanoate |
| 2106. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-[(2-oxo-1,3-dioxolan-4- |
| | yl)methyl]benzamide |
| 2107. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(2-methanesulfonylethyl)-3-methoxybenzamide |
| 2108. | 1-(acetyloxy)-3-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]propan-2-yl acetate |
| 2109. | 1-[(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxyphenyl)formamido]-3- |
| | (propanoyloxy)propan-2-yl propanoate |
| 2110. | 2-[(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxyphenyl)formamido]propyl 2- |
| | methylpropanoate |
| 2111. | (S)-5-ethoxy-2-(4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxybenzamido)-5- |
| | oxopentanoic acid |
| 2112. | (4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-3-methoxybenzoyl)-L-glutamine |
| 2113. | (S)-2-(4-((3-(4-(((3S,4R)-3-fluoro-1-methylpiperidin-4-yl)amino)-1-(2,2,2-trifluoroethyl)- |
| | 1H-indol-2-yl)prop-2-yn-1-yl)amino)-3-methoxybenzamido)-5-methoxy-5-oxopentanoic |
| | acid |
| 2114. | (S)-1-((3S,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2115. | (S)-1-((3R,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2116. | (S)-1-((3R,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2117. | (S)-1-((3S,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2118. | 1-((3S,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2119. | 1-((3R,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2120. | (R)-1-((3S,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2121. | (R)-1-((3R,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2122. | (R)-1-((3R,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2123. | (R)-1-((3S,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2124. | 4-((3-(4-(((3R,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3- |
| | methoxybenzenesulfonamide |
| 2125. | 1-(3,3-difluoro-4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)-3-methoxypropan-2-ol |
| 2126. | 2-{3-[(4-methanesulfonyl-2-methoxyphenyl)amino]prop-1-yn-1-yl}-N-(1-methylazepan- |
| | 4-yl)-1-(2,2,2-trifluoroethyl)-1H-indol-4-amine |
| 2127. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]azepan-1-yl}-3-methoxypropan-2-ol |
| 2128. | 2-{3-[(4-methanesulfonyl-2-methoxyphenyl)amino]prop-1-yn-1-yl}-N-{8-methyl-8- |
| | azabicyclo[3.2.1]octan-3-yl}-1-(2,2,2-trifluoroethyl)-1H-indol-4-amine |
| 2129. | 2-(3-{[4-methanesulfonyl-2-(trifluoromethoxy)phenyl]amino}prop-1-yn-1-yl)-N-(oxan-4- |
| | yl)-1-(2,2,2-trifluoroethyl)-1H-indol-4-amine |
| 2130. | 3-methoxy-4-[(3-{4-[(oxan-4-yl)amino]-1-(2,2,2-trifluoroethyl)-1H-indol-2-yl}prop-2- |
| | yn-1-yl)amino]-N-(1,2-oxazol-3-yl)benzene-1-sulfonamide |
| 2131. | 3-methoxy-N-(oxan-4-yl)-4-[(3-{4-[(oxan-4-yl)amino]-1-(2,2,2-trifluoroethyl)-1H-indol- |
| | 2-yl}prop-2-yn-1-yl)amino]benzene-1-sulfonamide |
| 2132. | 3-methoxy-N-(5-methyl-1,2-oxazol-3-yl)-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2133. | 2-{4-methoxy-5-[(3-{4-[(oxan-4-yl)amino]-1-(2,2,2-trifluoroethyl)-1H-indol-2-yl}prop-2- |
| | yn-1-yl)amino]pyridin-2-yl}-2-methylpropanenitrile |
| 2134. | N-(2-hydroxyethyl)-3-methoxy-N-methyl-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2135. | 6-fluoro-2-{3-[(4-methanesulfonyl-2-methoxyphenyl)amino]prop-1-yn-1-yl}-N-(oxan-4- |
| | yl)-1-(2,2,2-trifluoroethyl)-1H-indol-4-amine |
| 2136. | 4-[(3-{6-fluoro-4-[(oxan-4-yl)amino]-1-(2,2,2-trifluoroethyl)-1H-indol-2-yl}prop-2-yn-1- |
| | yl)amino]-3-methoxybenzene-1-sulfonamide |
| 2137. | 3-methoxy-N,N-dimethyl-4-[(3-{4-[(oxan-4-yl)amino]-1-(2,2,2-trifluoroethyl)-1H-indol- |
| | 2-yl}prop-2-yn-1-yl)amino]benzene-1-sulfonamide |
| 2138. | 3-methoxy-N-(2-methoxyethyl)-N-methyl-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2139. | 1-(4-{3-methoxy-4-[(3-{4-[(oxan-4-yl)amino]-1-(2,2,2-trifluoroethyl)-1H-indol-2- |
| | yl}prop-2-yn-1-yl)amino]benzenesulfonyl}piperazin-1-yl)ethan-1-one |
| 2140. | 2-(3-{[2-methoxy-4-(morpholine-4-sulfonyl)phenyl]amino}prop-1-yn-1-yl)-N-(oxan-4- |
| | yl)-1-(2,2,2-trifluoroethyl)-1H-indol-4-amine |
| 2141. | 3-methoxy-N-methyl-4-[(3-{4-[(oxan-4-yl)amino]-1-(2,2,2-trifluoroethyl)-1H-indol-2- |
| | yl}prop-2-yn-1-yl)amino]benzamide |
| 2142. | 3-methoxy-4-((3-(4-((tetrahydro-2H-pyran-4-yl)amino)-1-(2,2,2-trifluoroethyl)-1H-indol- |
| | 2-yl)prop-2-yn-1-yl)amino)benzoic acid |
| 2143. | methyl 3-methoxy-4-((3-(4-((tetrahydro-2H-pyran-4-yl)amino)-1-(2,2,2-trifluoroethyl)- |
| | 1H-indol-2-yl)prop-2-yn-1-yl)amino)benzoate |
| 2144. | 3-methoxy-4-((3-(4-((tetrahydro-2H-pyran-4-yl)amino)-1-(2,2,2-trifluoroethyl)-1H-indol- |
| | 2-yl)prop-2-yn-1-yl)amino)benzamide |
| 2145. | 3-methoxy-N-(2-methoxyethyl)-N-methyl-4-((3-(4-((tetrahydro-2H-pyran-4-yl)amino)-1- |
| | (2,2,2-trifluoroethyl)-1H-indol-2-yl)prop-2-yn-1-yl)amino)benzenesulfonamide |
| 2146. | N,N-bis(2-hydroxyethyl)-3-methoxy-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2147. | 3-methoxy-N-methyl-4-[(3-{4-[(oxan-4-yl)amino]-1-(2,2,2-trifluoroethyl)-1H-indol-2- |
| | yl}prop-2-yn-1-yl)amino]benzene-1-sulfonamide |
| 2148. | 5-methanesulfonyl-2-[(3-{4-[(oxan-4-yl)amino]-1-(2,2,2-trifluoroethyl)-1H-indol-2- |
| | yl}prop-2-yn-1-yl)amino]phenol |
| 2149. | 3-methoxy-4-[(3-{5-methoxy-4-[(oxan-4-yl)amino]-1-(2,2,2-trifluoroethyl)-1H-indol-2- |
| | yl}prop-2-yn-1-yl)amino]benzene-1-sulfonamide |
| 2150. | 2-[3-({2-methoxy-4-[(4-methylpiperazin-1-yl)sulfonyl]phenyl}amino)prop-1-yn-1-yl]-N- |
| | (oxan-4-yl)-1-(2,2,2-trifluoroethyl)-1H-indol-4-amine |
| 2151. | 2-{3-[(4-methanesulfonyl-2-methoxyphenyl)amino]prop-1-yn-1-yl}-5-methoxy-N-(oxan- |
| | 4-yl)-1-(2,2,2-trifluoroethyl)-1H-indol-4-amine |
| 2152. | 3-methoxy-4-[(3-{6-methoxy-4-[(oxan-4-yl)amino]-1-(2,2,2-trifluoroethyl)-1H-indol-2- |
| | yl}prop-2-yn-1-yl)amino]benzene-1-sulfonamide |
| 2153. | 5-methanesulfonyl-2-[(3-{4-[(oxan-4-yl)amino]-1-(2,2,2-trifluoroethyl)-1H-indol-2- |
| | yl}prop-2-yn-1-yl)amino]phenyl propanoate |
| 2154. | N-(2-hydroxyethyl)-3-methoxy-4-((3-(4-((tetrahydro-2H-pyran-4-yl)amino)-1-(2,2,2- |
| | trifluoroethyl)-1H-indol-2-yl)prop-2-yn-1-yl)amino)benzenesulfonamide |
| 2155. | 2-{3-[(4-methanesulfonyl-2-methoxyphenyl)amino]prop-1-yn-1-yl}-N-[(2S,4R)-2- |
| | methyloxan-4-yl]-1-(2,2,2-trifluoroethyl)-1H-indol-4-amine |
| 2156. | 2-{3-[(4-methanesulfonyl-2-methoxyphenyl)amino]prop-1-yn-1-yl}-N-[(2S,4S)-2- |
| | methyloxan-4-yl]-1-(2,2,2-trifluoroethyl)-1H-indol-4-amine |
| 2157. | 3-methoxy-4-{[3-(4-{[(2S,4R)-2-methyloxan-4-yl]amino}-1-(2,2,2-trifluoroethyl)-1H- |
| | indol-2-yl)prop-2-yn-1-yl]amino}benzene-1-sulfonamide |
| 2158. | 3-methoxy-4-{[3-(4-{[(2S,4S)-2-methyloxan-4-yl]amino}-1-(2,2,2-trifluoroethyl)-1H- |
| | indol-2-yl)prop-2-yn-1-yl]amino}benzene-1-sulfonamide |
| 2159. | 3-methoxy-4-{[3-(4-{[(2S,4R)-2-methyloxan-4-yl]amino}-1-(2,2,2-trifluoroethyl)-1H- |
| | indol-2-yl)prop-2-yn-1-yl]amino}benzamide |
| 2160. | 3-methoxy-4-{[3-(4-{[(2S,4S)-2-methyloxan-4-yl]amino}-1-(2,2,2-trifluoroethyl)-1H- |
| | indol-2-yl)prop-2-yn-1-yl]amino}benzamide |
| 2161. | rac-N-[(3R,4R)-3-fluorooxan-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 2162. | 2-(3-((2-methoxy-4-(methylsulfonyl)phenyl)amino)prop-1-yn-1-yl)-N-((3R,4S)-3- |
| | methoxytetrahydro-2H-pyran-4-yl)-1-(2,2,2-trifluoroethyl)-1H-indol-4-amine |
| 2163. | 2-(3-((2-methoxy-4-(methylsulfonyl)phenyl)amino)prop-1-yn-1-yl)-N-((3S,4S)-3- |
| | methoxytetrahydro-2H-pyran-4-yl)-1-(2,2,2-trifluoroethyl)-1H-indol-4-amine |
| 2164. | N-[(3S,4S)-3-fluorooxan-4-yl]-2-{3-[(4-methanesulfonyl-2-methoxyphenyl)amino]prop- |
| | 1-yn-1-yl}-1-(2,2,2-trifluoroethyl)-1H-indol-4-amine |
| 2165. | N-[(3R,4R)-3-fluorooxan-4-yl]-2-{3-[(4-methanesulfonyl-2-methoxyphenyl)amino]prop- |
| | 1-yn-1-yl}-1-(2,2,2-trifluoroethyl)-1H-indol-4-amine |
| 2166. | 2-{3-[(4-methanesulfonyl-2-methoxyphenyl)amino]prop-1-yn-1-yl}-N-[(2R,4R,6S)-2,6- |
| | dimethyloxan-4-yl]-1-(2,2,2-trifluoroethyl)-1H-indol-4-amine |
| 2167. | 2-{3-[(4-methanesulfonyl-2-methoxyphenyl)amino]prop-1-yn-1-yl}-N-[(2R,4S,6S)-2,6- |
| | dimethyloxan-4-yl]-1-(2,2,2-trifluoroethyl)-1H-indol-4-amine |
| 2168. | 3-methoxy-4-{[3-(4-{[(2R,4R,6S)-2,6-dimethyloxan-4-yl]amino}-1-(2,2,2-trifluoroethyl)- |
| | 1H-indol-2-yl)prop-2-yn-1-yl]amino}benzamide |
| 2169. | 3-methoxy-4-{[3-(4-{[(2R,4S,6S)-2,6-dimethyloxan-4-yl]amino}-1-(2,2,2-trifluoroethyl)- |
| | 1H-indol-2-yl)prop-2-yn-1-yl]amino}benzamide |
| 2170. | 3-methoxy-4-{[3-(4-{[(2R,4S,6S)-2,6-dimethyloxan-4-yl]amino}-1-(2,2,2-trifluoroethyl)- |
| | 1H-indol-2-yl)prop-2-yn-1-yl]amino}benzene-1-sulfonamide |
| 2171. | 3-methoxy-4-{[3-(4-{[(2R,4R,6S)-2,6-dimethyloxan-4-yl]amino}-1-(2,2,2-trifluoroethyl)- |
| | 1H-indol-2-yl)prop-2-yn-1-yl]amino}benzene-1-sulfonamide |
| 2172. | 4-((2-(3-((4-methoxypyridin-3-yl)amino)prop-1-yn-1-yl)-1-(2,2,2-trifluoroethyl)-1H- |
| | indol-4-yl)amino)tetrahydro-2H-thiopyran 1,1-dioxide |
| 2173. | 2-{5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-4-methoxypyridin-2-yl}-2-methylpropanenitrile |
| 2174. | 4-{[2-(3-{[4-methanesulfonyl-2-(trifluoromethoxy)phenyl]amino}prop-1-yn-1-yl)-1- |
| | (2,2,2-trifluoroethyl)-1H-indol-4-yl]amino}-1λ⁶-thiane-1,1-dione |
| 2175. | 4-{[2-(3-{[2-methoxy-4-(morpholine-4-sulfonyl)phenyl]amino}prop-1-yn-1-yl)-1-(2,2,2- |
| | trifluoroethyl)-1H-indol-4-yl]amino}-1λ⁶-thiane-1,1-dione |
| 2176. | 4-({2-[3-({2-[2-(dimethylamino)ethoxy]-4-methanesulfonylphenyl}amino)prop-1-yn-1- |
| | yl]-1-(2,2,2-trifluoroethyl)-1H-indol-4-yl}amino)-1λ⁶-thiane-1,1-dione |
| 2177. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(2-hydroxyethyl)-3-methoxy-N-methylbenzene-1-sulfonamide |
| 2178. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(1,2-oxazol-3-yl)benzene-1-sulfonamide |
| 2179. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(5-methyl-1,2-oxazol-3-yl)benzene-1-sulfonamide |
| 2180. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(oxan-4-yl)benzene-1-sulfonamide |
| 2181. | N-(2,3-dihydroxypropyl)-4-[(3-{4-[(1,1-dioxo-1λ⁶-thian-4-yl)amino]-1-(2,2,2- |
| | trifluoroethyl)-1H-indol-2-yl}prop-2-yn-1-yl)amino]-3-methoxybenzene-1-sulfonamide |
| 2182. | N-[2-(dimethylamino)ethyl]-4-[(3-{4-[(1,1-dioxo-1λ⁶-thian-4-yl)amino]-1-(2,2,2- |
| | trifluoroethyl)-1H-indol-2-yl}prop-2-yn-1-yl)amino]-3-methoxy-N-methylbenzene-1- |
| | sulfonamide |
| 2183. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N,N-dimethylbenzene-1-sulfonamide |
| 2184. | 4-({2-[3-({4-[(4-acetylpiperazin-1-yl)sulfonyl]-2-methoxyphenyl}amino)prop-1-yn-1-yl]- |
| | 1-(2,2,2-trifluoroethyl)-1H-indol-4-yl}amino)-1λ⁶-thiane-1,1-dione |
| 2185. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(2-methoxyethyl)-N-methylbenzene-1-sulfonamide |
| 2186. | 4-[(2-{3-[(2-methoxy-4-{2-oxa-6-azaspiro[3.3]heptane-6-sulfonyl}phenyl)amino]prop-1- |
| | yn-1-yl}-1-(2,2,2-trifluoroethyl)-1H-indol-4-yl)amino]-1λ⁶-thiane-1,1-dione |
| 2187. | 4-({2-[3-({2-methoxy-4-[(4-methylpiperazin-1-yl)sulfonyl]phenyl}amino)prop-1-yn-1- |
| | yl]-1-(2,2,2-trifluoroethyl)-1H-indol-4-yl}amino)-1λ⁶-thiane-1,1-dione |
| 2188. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-methylbenzamide |
| 2189. | 4-((2-(3-((4-methoxy-6-(methylsulfonyl)pyridin-3-yl)amino)prop-1-yn-1-yl)-1-(2,2,2- |
| | trifluoroethyl)-1H-indol-4-yl)amino)tetrahydro-2H-thiopyran 1,1-dioxide |
| 2190. | 4-((2-(3-((4-(methylsulfonyl)-2-(2,2,2-trifluoroethoxy)phenyl)amino)prop-1-yn-1-yl)-1- |
| | (2,2,2-trifluoroethyl)-1H-indol-4-yl)amino)tetrahydro-2H-thiopyran 1,1-dioxide |
| 2191. | 2-(2-((3-(4-((1,1-dioxidotetrahydro-2H-thiopyran-4-yl)amino)-1-(2,2,2-trifluoroethyl)- |
| | 1H-indol-2-yl)prop-2-yn-1-yl)amino)-5-(methylsulfonyl)phenoxy)acetamide |
| 2192. | 2-(2-((3-(4-((1,1-dioxidotetrahydro-2H-thiopyran-4-yl)amino)-1-(2,2,2-trifluoroethyl)- |
| | 1H-indol-2-yl)prop-2-yn-1-yl)amino)-5-(methylsulfonyl)phenoxy)acetamide |

TABLE 2
Aryl-linked indole compounds of the disclosure.

| Mol # | IUPAC name |
|---|---|
| 2193. | 4-{4-[(1,1-dioxo-1λ⁶-thian-4-yl)amino]-1-(2,2,2-trifluoroethyl)-1H-indol-2- |
| | yl}benzamide |
| 2194. | 4-({2-[4-(aminomethyl)phenyl]-1-(2,2,2-trifluoroethyl)-1H-indol-4-yl}amino)-1λ⁶- |
| | thiane-1,1-dione |
| 2195. | 4-[(2-{4-[(methylamino)methyl]phenyl}-1-(2,2,2-trifluoroethyl)-1H-indol-4- |
| | yl)amino]-1λ⁶-thiane-1,1-dione |
| 2196. | tert-butyl N-[(4-{4-[(1,1-dioxo-1λ⁶-thian-4-yl)amino]-1-(2,2,2-trifluoroethyl)-1H- |
| | indol-2-yl}phenyl)methyl]-N-methylcarbamate |
| 2197. | 4-{4-[(1,1-dioxo-1λ⁶-thian-4-yl)amino]-1-(2,2,2-trifluoroethyl)-1H-indol-2-yl}-N- |
| | methylbenzamide |
| 2198. | tert-butyl N-[(4-{4-[(1,1-dioxo-1λ⁶-thian-4-yl)amino]-1-(2,2,2-trifluoroethyl)-1H- |
| | indol-2-yl}phenyl)methyl]carbamate |
| 2199. | 2-(5-{[(4-{4-[(1,1-dioxo-1λ⁶-thian-4-yl)amino]-1-(2,2,2-trifluoroethyl)-1H-indol-2- |
| | yl}phenyl)methyl]amino}pyridin-2-yl)-2-methylpropanenitrile |
| 2200. | 4-{[2-(4-{[(4-methanesulfonylphenyl)amino]methyl}phenyl)-1-(2,2,2-trifluoroethyl)- |
| | 1H-indol-4-yl]amino}-1λ⁶-thiane-1,1-dione |
| 2201. | 4-[(2-{4-[(phenylamino)methyl]phenyl}-1-(2,2,2-trifluoroethyl)-1H-indol-4- |
| | yl)amino]-1λ⁶-thiane-1,1-dione |
| 2202. | 3-{4-[(1,1-dioxo-1λ⁶-thian-4-yl)amino]-1-(2,2,2-trifluoroethyl)-1H-indol-2- |
| | yl}benzonitrile |
| 2203. | 4-{[2-(2-fluoro-4-methylphenyl)-1-(2,2,2-trifluoroethyl)-1H-indol-4-yl]amino}-1λ⁶- |
| | thiane-1,1-dione |
| 2204. | 4-{[2-(3-chlorophenyl)-1-(2,2,2-trifluoroethyl)-1H-indol-4-yl]amino}-1λ⁶-thiane-1,1- |
| | dione |
| 2205. | -{[2-(3-methoxyphenyl)-1-(2,2,2-trifluoroethyl)-1H-indol-4-yl]amino}-1λ⁶-thiane-1,1- |
| | dione |
| 2206. | 4-{[2-(4-chlorophenyl)-1-(2,2,2-trifluoroethyl)-1H-indol-4-yl]amino}-1λ⁶-thiane-1,1- |
| | dione |
| 2207. | 4-tert-butyl-N-[(4-{4-[(1,1-dioxo-1λ⁶-thian-4-yl)amino]-1-(2,2,2-trifluoroethyl)-1H- |
| | indol-2-yl}phenyl)methyl]benzamide |
| 2208. | 4-cyano-N-[(4-{4-[(1,1-dioxo-1λ⁶-thian-4-yl)amino]-1-(2,2,2-trifluoroethyl)-1H- |
| | indol-2-yl}phenyl)methyl]benzamide |
| 2209. | 4-chloro-N-[(4-{4-[(1,1-dioxo-1λ⁶-thian-4-yl)amino]-1-(2,2,2-trifluoroethyl)-1H- |
| | indol-2-yl}phenyl)methyl]benzamide |
| 2210. | 3-[6-(1-cyano-1-methylethyl)pyridin-3-yl]-1-[(4-{4-[(1,1-dioxo-1λ⁶-thian-4- |
| | yl)amino]-1-(2,2,2-trifluoroethyl)-1H-indol-2-yl}phenyl)methyl]urea |
| 2211. | 3-[(4-{4-[(1,1-dioxo-1λ⁶-thian-4-yl)amino]-1-(2,2,2-trifluoroethyl)-1H-indol-2- |
| | yl}phenyl)methyl]-1-phenylurea |
| 2212. | 3-{4-[(1,1-dioxo-1λ⁶-thian-4-yl)amino]-1-(2,2,2-trifluoroethyl)-1H-indol-2- |
| | yl}benzoic acid |
| 2213. | 4-({2-[3-(dimethylamino)phenyl]-1-(2,2,2-trifluoroethyl)-1H-indol-4-yl}amino)-1λ⁶- |
| | thiane-1,1-dione |
| 2214. | 3-{4-[(1,1-dioxo-1λ⁶-thian-4-yl)amino]-1-(2,2,2-trifluoroethyl)-1H-indol-2-yl}-N- |
| | methylbenzamide |
| 2215. | 4-{4-[(11-dioxo-1λ⁶-thian-4-yl)amino]-1-(2,2,2-trifluoroethyl)-1H-indol-2-yl}benzoic |
| | acid |
| 2216. | 4-[(2-{4-[(morpholin-4-yl)methyl]phenyl}-1-(2,2,2-trifluoroethyl)-1H-indol-4- |
| | yl)amino]-1λ⁶-thiane-1,1-dione |
| 2217. | methyl N-(3-{4-[(1,1-dioxo-1λ⁶-thian-4-yl)amino]-1-(2,2,2-trifluoroethyl)-1H-indol-2- |
| | yl}phenyl)carbamate |
| 2218. | 1-(4-{4-[(1,1-dioxo-1λ⁶-thian-4-yl)amino]-1-(2,2,2-trifluoroethyl)-1H-indol-2- |
| | yl}phenyl)cyclopropane-1-carbonitrile |
| 2219. | 4-({2-[4-(hydroxymethyl)phenyl]-1-(2,2,2-trifluoroethyl)-1H-indol-4-yl}amino)-1λ⁶- |
| | thiane-1,1-dione |
| 2220. | 1-[(4-{4-[(1,1-dioxo-1λ⁶-thian-4-yl)amino]-1-(2,2,2-trifluoroethyl)-1H-indol-2- |
| | yl}phenyl)methyl]-3-(4-methanesulfonylphenyl)urea |
| 2221. | 4-{[2-(4-{[(6-methanesulfonylpyridin-3-yl)amino]methyl}phenyl)-1-(2,2,2- |
| | trifluoroethyl)-1H-indol-4-yl]amino}-1λ⁶-thiane-1,1-dione |
| 2222. | 2-(4-{[(4-methanesulfonylphenyl)amino]methyl}phenyl)-N-[1-(oxan-4-yl)piperidin-4- |
| | yl]-1-(2,2,2-trifluoroethyl)-1H-indol-4-amine |
| 2223. | 2-(4-{[(6-methylpyridin-3-yl)amino]methyl}phenyl)-N-[1-(oxan-4-yl)piperidin-4-yl]- |
| | 1-(2,2,2-trifluoroethyl)-1H-indol-4-amine |
| 2224. | 2-(4-{[(4-chlorophenyl)amino]methyl}phenyl)-N-[1-(oxan-4-yl)piperidin-4-yl]-1- |
| | (2,2,2-trifluoroethyl)-1H-indol-4-amine |
| 2225. | 2-(4-{[(4-methoxyphenyl)amino]methyl}phenyl)-N-[1-(oxan-4-yl)piperidin-4-yl]-1- |
| | (2,2,2-trifluoroethyl)-1H-indol-4-amine |
| 2226. | 2-(4-{[(3-chlorophenyl)amino]methyl}phenyl)-N-[1-(oxan-4-yl)piperidin-4-yl]-1- |
| | (2,2,2-trifluoroethyl)-1H-indol-4-amine |
| 2227. | 6-methyl-N-{[4-(5-{[(1-methylpiperidin-4-yl)amino]methyl}-1-(2,2,2-trifluoroethyl)- |
| | 1H-indol-2-yl)phenyl]methyl}pyridin-3-amine |
| 2228. | N-{[2-(4-{[(4-methanesulfonylphenyl)amino]methyl}phenyl)-1-(2,2,2-trifluoroethyl)- |
| | 1H-indol-5-yl]methyl}-1-methylpiperidin-4-amine |
| 2229. | 2-(5-amino-[1,1′-biphenyl]-3-yl)-N-(1-methylpiperidin-4-yl)-1-(2,2,2-trifluoroethyl)- |
| | 1H-indol-4-amine |
| 2230. | 2-{4-[amino(phenyl)methyl]phenyl}-N-(1-methylpiperidin-4-yl)-1-(2,2,2- |
| | trifluoroethyl)-1H-indol-4-amine |
| 2231. | 2-(4-(amino(cyclohexyl)methyl)phenyl)-N-(1-methylpiperidin-4-yl)-1-(2,2,2- |
| | trifluoroethyl)-1H-indol-4-amine |
| 2232. | 2-{4-[(cyclopentylamino)methyl]phenyl}-N-(1-methylpiperidin-4-yl)-1-(2,2,2- |
| | trifluoroethyl)-1H-indol-4-amine |
| 2233. | 2-(4-{1-[(4-methanesulfonylphenyl)amino]ethyl}phenyl)-N-(1-methylpiperidin-4-yl)- |
| | 1-(2,2,2-trifluoroethyl)-1H-indol-4-amine |
| 2234. | (+/−)-2-{4-[(cyclopropylamino)methyl]phenyl}-N-[(3R,4S)-3-fluoro-1- |
| | methylpiperidin-4-yl]-1-(2,2,2-trifluoroethyl)-1H-indol-4-amine |
| 2235. | (+/−)-N-[(3R,4S)-3-fluoro-1-methylpiperidin-4-yl]-2-(4-{[(4-methanesulfonyl-2- |
| | methoxyphenyl)amino]methyl}phenyl)-1-(2,2,2-trifluoroethyl)-1H-indol-4-amine |
| 2236. | N-[(3R,4S)-3-fluoro-1-methylpiperidin-4-yl]-2-(4-{[(4- |
| | methanesulfonylphenyl)amino]methyl}phenyl)-1-(2,2,2-trifluoroethyl)-1H-indol-4- |
| | amine |
| 2237. | (+/−)-N-{[4-(4-{[(3R,4S)-3-fluoro-1-methylpiperidin-4-yl]amino}-1-(2,2,2- |
| | trifluoroethyl)-1H-indol-2-yl)phenyl]methyl}benzamide |
| 2238. | (+/−)-N-{[4-(4-{[(3R,4S)-3-fluoro-1-methylpiperidin-4-yl]amino}-1-(2,2,2- |
| | trifluoroethyl)-1H-indol-2-yl)phenyl]methyl}cyclopropanecarboxamide |
| 2239. | 1-methoxy-3-(4-{[2-(3-methyl-2H-indazol-6-yl)-1-(2,2,2-trifluoroethyl)-1H-indol-4- |
| | yl]amino}piperidin-1-yl)propan-2-ol |
| 2240. | 1-(4-{[2-(2H-indazol-6-yl)-1-(2,2,2-trifluoroethyl)-1H-indol-4-yl]amino}piperidin-1- |
| | yl)-3-methoxypropan-2-ol |
| 2241. | 4-{[1-(2-hydroxy-3-methoxypropyl)piperidin-4-yl]amino}-1-(2,2,2-trifluoroethyl)- |
| | 2′,3′-dihydro-1H,1′H-[2,6′-biindol]-2′-one |
| 2242. | 4-(4-((1-(2-hydroxy-3-methoxypropyl)piperidin-4-yl)amino)-1-(2,2,2-trifluoroethyl)- |
| | 1H-indol-2-yl)isoindolin-1-one |
| 2243. | N-[3-(4-{[1-(2-hydroxy-3-methoxypropyl)piperidin-4-yl]amino}-1-(2,2,2- |
| | trifluoroethyl)-1H-indol-2-yl)phenyl]acetamide |

TABLE 3
Heteroaryl-linked indole compounds of the disclosure

| Mol # | IUPAC name |
|---|---|
| 2244. | 4-((2-(6-methoxypyridin-3-yl)-1-(2,2,2-trifluoroethyl)-1H-indol-4- |
| | yl)amino)tetrahydro-2H-thiopyran 1,1-dioxide-indol-4-amine |
| 2245. | 4-((2-(6-methylpyridin-3-yl)-1-(2,2,2-trifluoroethyl)-1H-indol-4-yl)amino)tetrahydro- |
| | 2H-thiopyran 1,1-dioxide |
| 2246. | 4-((2-(6-(dimethylamino)pyridin-3-yl)-1-(2,2,2-trifluoroethyl)-1H-indol-4- |
| | yl)amino)tetrahydro-2H-thiopyran 1,1-dioxide |
| 2247. | 4-((2-(quinolin-4-yl)-1-(2,2,2-trifluoroethyl)-1H-indol-4-yl)amino)tetrahydro-2H- |
| | thiopyran 1,1-dioxide |
| 2248. | 4-((2-(2-fluoropyridin-3-yl)-1-(2,2,2-trifluoroethyl)-1H-indol-4-yl)amino)tetrahydro- |
| | 2H-thiopyran 1,1-dioxide |
| 2249. | 1-(4-((2-(5-aminopyridin-3-yl)-1-(2,2,2-trifluoroethyl)-1H-indol-4- |
| | yl)amino)piperidin-1-yl)-3-methoxypropan-2-ol |
| 2250. | 2-(2-amino-6-phenylpyridin-4-yl)-N-(1-methylpiperidin-4-yl)-1-(2,2,2-trifluoroethyl)- |
| | 1H-indol-4-amine |
| 2251. | 5-(4-((1,1-dioxidotetrahydro-2H-thiopyran-4-yl)amino)-1-(2,2,2-trifluoroethyl)-1H- |
| | indol-2-yl)-N-methylpicolinamide |
| 2252. | 2-(2-amino-6-phenylpyrimidin-4-yl)-N-(1-methylpiperidin-4-yl)-1-(2,2,2- |
| | trifluoroethyl)-1H-indol-4-amine |
| 2253. | 2-(2-amino-6-(cyclohex-1-en-1-yl)pyrimidin-4-yl)-N-(1-methylpiperidin-4-yl)-1- |
| | (2,2,2-trifluoroethyl)-1H-indol-4-amine |
| 2254. | 2-(2-amino-6-cyclohexylpyrimidin-4-yl)-N-(1-methylpiperidin-4-yl)-1-(2,2,2- |
| | trifluoroethyl)-1H-indol-4-amine |
| 2255. | 2-(2-(methylamino)pyrimidin-4-yl)-N-(1-methylpiperidin-4-yl)-1-(2,2,2- |
| | trifluoroethyl)-1H-indol-4-amine |
| 2256. | 2-(2-aminopyrimidin-4-yl)-N-(1-methylpiperidin-4-yl)-1-(2,2,2-trifluoroethyl)-1H- |
| | indol-4-amine |
| 2257. | 4-{[2-(1-methyl-1H-pyrazol-3-yl)-1-(2,2,2-trifluoroethyl)-1H-indol-4-yl]amino}-1λ⁶- |
| | thiane-1,1-dione |
| 2258. | 4-({2-[1-(2-hydroxyethyl)-1H-pyrazol-4-yl]-1-(2,2,2-trifluoroethyl)-1H-indol-4- |
| | yl}amino)-1λ⁶-thiane-1,1-dione |
| 2259. | 4-[(2-{1-[(pyridin-3-yl)methyl]-1H-pyrazol-4-yl}-1-(2,2,2-trifluoroethyl)-1H-indol-4- |
| | yl)amino]-1λ⁶-thiane-1,1-dione |
| 2260. | 4-((2-(1-(2-methoxyethyl)-1H-pyrazol-4-yl)-1-(2,2,2-trifluoroethyl)-1H-indol-4- |
| | yl)amino)tetrahydro-2H-thiopyran 1,1-dioxide |
| 2261. | 4-[(2-{1-[(pyridin-4-yl)methyl]-1H-pyrazol-4-yl}-1-(2,2,2-trifluoroethyl)-1H-indol-4- |
| | yl)amino]-1λ⁶-thiane-1,1-dione |
| 2262. | 2-(5-amino-1-phenyl-1H-pyrazol-3-yl)-N-(1-methylpiperidin-4-yl)-1-(2,2,2- |
| | trifluoroethyl)-1H-indol-4-amine |
| 2263. | 2-{5-[amino(phenyl)methyl]-1H-pyrazol-3-yl}-N-(1-methylpiperidin-4-yl)-1-(2,2,2- |
| | trifluoroethyl)-1H-indol-4-amine |
| 2264. | 2-(5-amino-1H-pyrazol-3-yl)-N-(1-methylpiperidin-4-yl)-1-(2,2,2-trifluoroethyl)-1H- |
| | indol-4-amine |
| 2265. | 2-(5-{[(4-methanesulfonylphenyl)amino]methyl}-1H-pyrazol-3-yl)-N-(1- |
| | methylpiperidin-4-yl)-1-(2,2,2-trifluoroethyl)-1H-indol-4-amine |
| 2266. | 2-[5-(methylamino)-1H-pyrazol-3-yl]-N-(1-methylpiperidin-4-yl)-1-(2,2,2- |
| | trifluoroethyl)-1H-indol-4-amine |
| 2267. | 2-(5-{[(4-methanesulfonylphenyl)amino]methyl}-1,3,4-thiadiazol-2-yl)-N-(1- |
| | methylpiperidin-4-yl)-1-(2,2,2-trifluoroethyl)-1H-indol-4-amine |
| 2268. | N-{[5-(4-{[(3R,4S)-3-fluoro-1-methylpiperidin-4-yl]amino}-1-(2,2,2-trifluoroethyl)- |
| | 1H-indol-2-yl)-1,3,4-thiadiazol-2-yl]methyl}cyclopropanecarboxamide |
| 2269. | N-{[5-(4-{[(3R,4S)-3-fluoro-1-methylpiperidin-4-yl]amino}-1-(2,2,2-trifluoroethyl)- |
| | 1H-indol-2-yl)-1,3,4-thiadiazol-2-yl]methyl}benzamide |
| 2270. | (+/−)-N-[(3R,4S)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2271. | (+/−)-N-[(3R,4R)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2272. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}cyclopentanecarboxamide |
| 2273. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}thiophene-2-carboxamide |
| 2274. | 1-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]cyclopropane-1-carboxamide |
| 2275. | (+/−)-2,2-difluoro-N-[(5-{4-[(1-methylpiperidin-4-yl)amino]-1-(2,2,2-trifluoroethyl)- |
| | 1H-indol-2-yl}-1,3,4-thiadiazol-2-yl)methyl]cyclopropane-1-carboxamide |
| 2276. | (+/−)-(1R,2S)-2-methyl-N-[(5-{4-[(1-methylpiperidin-4-yl)amino]-1-(2,2,2- |
| | trifluoroethyl)-1H-indol-2-yl}-1,3,4-thiadiazol-2-yl)methyl]cyclopropane-1- |
| | carboxamide |
| 2277. | (+/−)-(1R,2R)-2-methyl-N-[(5-{4-[(1-methylpiperidin-4-yl)amino]-1-(2,2,2- |
| | trifluoroethyl)-1H-indol-2-yl}-1,3,4-thiadiazol-2-yl)methyl]cyclopropane-1- |
| | carboxamide |
| 2278. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]cyclopropanecarboxamide |
| 2279. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}oxetane-3-carboxamide |
| 2280. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}cyclobutanecarboxamide |
| 2281. | (+/−)-methyl N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}carbamate |
| 2282. | methyl 4-{[(5-{4-[(1-methylpiperidin-4-yl)amino]-1-(2,2,2-trifluoroethyl)-1H-indol-2- |
| | yl}-1,3,4-thiadiazol-2-yl)methyl]carbamoyl}benzoate |
| 2283. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}-1-methylpiperidine-4- |
| | carboxamide |
| 2284. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]pyridine-2-carboxamide |
| 2285. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]pyridine-3-carboxamide |
| 2286. | (+/−)-N-[(3R,4S)-3-fluoro-1-methylpiperidin-4-yl]-2-{5-[(methylamino)methyl]-1,3,4- |
| | thiadiazol-2-yl}-1-(2,2,2-trifluoroethyl)-1H-indol-4-amine |
| 2287. | (+/−)-benzyl N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}carbamate |
| 2288. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-4-[(morphohn-4-yl)methyl]benzamide |
| 2289. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-3-[(morpholin-4-yl)methyl]benzamide |
| 2290. | N-{[5-(4-{[(3S,4R)-3-fluoro-1-methylpiperidin-4-yl]amino}-1-(2,2,2-trifluoroethyl)- |
| | 1H-indol-2-yl)-1,3,4-thiadiazol-2-yl]methyl}cyclopropanecarboxamide |
| 2291. | N-{[5-(4-{[(3R,4S)-3-fluoro-1-methylpiperidin-4-yl]amino}-1-(2,2,2-trifluoroethyl)- |
| | 1H-indol-2-yl)-1,3,4-thiadiazol-2-yl]methyl}cyclopropanecarboxamide |
| 2292. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]pyridine-4-carboxamide |
| 2293. | 2-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]benzamide |
| 2294. | 3-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]benzamide |
| 2295. | 4-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]benzamide |
| 2296. | (+/−)-(1S,2S)-N-[(5-{4-[(1-methylpiperidin-4-yl)amino]-1-(2,2,2-trifluoroethyl)-1H- |
| | indol-2-yl}-1,3,4-thiadiazol-2-yl)methyl]-2-phenylcyclopropane-1-carboxamide |
| 2297. | 4-{[(5-{4-[(1-methylpiperidin-4-yl)amino]-1-(2,2,2-trifluoroethyl)-1H-indol-2-yl}- |
| | 1,3,4-thiadiazol-2-yl)methyl]carbamoyl}benzoic acid |
| 2298. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-indazole-5-carboxamide |
| 2299. | 3-methyl-1-[(5-{4-[(1-methylpiperidin-4-yl)amino]-1-(2,2,2-trifluoroethyl)-1H-indol- |
| | 2-yl}-1,3,4-thiadiazol-2-yl)methyl]urea |
| 2300. | 2-methyl-N-[(5-{4-[(1-methylpiperidin-4-yl)amino]-1-(2,2,2-trifluoroethyl)-1H-indol- |
| | 2-yl}-1,3,4-thiadiazol-2-yl)methyl]propanamide |
| 2301. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]acetamide |
| 2302. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-2-phenylacetamide |
| 2303. | 2-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]acetamide |
| 2304. | N-[(3R,4S)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2305. | N-[(3S,4R)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2306. | 4-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2307. | 3-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2308. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]butanamide |
| 2309. | 2-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2310. | 2-[5-(aminomethyl)-1,3,4-thiadiazol-2-yl]-N-(1-methylpiperidin-4-yl)-1-(2,2,2- |
| | trifluoroethyl)-1H-indol-4-amine |
| 2311. | 3,3-dimethyl-1-[(5-{4-[(1-methylpiperidin-4-yl)amino]-1-(2,2,2-trifluoroethyl)-1H- |
| | indol-2-yl}-1,3,4-thiadiazol-2-yl)methyl]urea |
| 2312. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-indazole-6-carboxamide |
| 2313. | benzyl N-{[5-(4-{[(3S,4R)-3-fluoropiperidin-4-yl]amino}-1-(2,2,2-trifluoroethyl)-1H- |
| | indol-2-yl)-1,3,4-thiadiazol-2-yl]methyl}carbamate |
| 2314. | 1-methyl-N-[(5-{4-[(1-methylpiperidin-4-yl)amino]-1-(2,2,2-trifluoroethyl)-1H-indol- |
| | 2-yl}-1,3,4-thiadiazol-2-yl)methyl]cyclopropane-1-carboxamide |
| 2315. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-pyrazole-4-carboxamide |
| 2316. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-pyrazole-5-carboxamide |
| 2317. | 1-ethyl-N-[(5-{4-[(1-methylpiperidin-4-yl)amino]-1-(2,2,2-trifluoroethyl)-1H-indol-2- |
| | yl}-1,3,4-thiadiazol-2-yl)methyl]cyclopropane-1-carboxamide |
| 2318. | (+/−)-methyl (1R,2R)-2-{[(5-{4-[(1-methylpiperidin-4-yl)amino]-1-(2,2,2- |
| | trifluoroethyl)-1H-indol-2-yl}-1,3,4-thiadiazol-2-yl)methyl]carbamoyl}cyclopropane- |
| | 1-carboxylate |
| 2319. | (+/−)-(1R,2R)-2-{[(5-{4-[(1-methylpiperidin-4-yl)amino]-1-(2,2,2-trifluoroethyl)-1H- |
| | indol-2-yl}-1,3,4-thiadiazol-2-yl)methyl]carbamoyl}cyclopropane-1-carboxylic acid |
| 2320. | 1-methyl-N-[(5-{4-[(1-methylpiperidin-4-yl)amino]-1-(2,2,2-trifluoroethyl)-1H-indol- |
| | 2-yl}-1,3,4-thiadiazol-2-yl)methyl]-1H-pyrrole-3-carboxamide |
| 2321. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-pyrrole-3-carboxamide |
| 2322. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-2-(thiophen-2-yl)cyclopropane-1-carboxamide |
| 2323. | N-(1-methylpiperidin-4-yl)-2-(5-{[(pyrrolidin-3-yl)amino]methyl}-1,3,4-thiadiazol-2- |
| | yl)-1-(2,2,2-trifluoroethyl)-1H-indol-4-amine |
| 2324. | (+/−)-(1R,2S)-2-{[(5-{4-[(1-methylpiperidin-4-yl)amino]-1-(2,2,2-trifluoroethyl)-1H- |
| | indol-2-yl}-1,3,4-thiadiazol-2-yl)methyl]carbamoyl}cyclopropane-1-carboxylic acid |
| 2325. | N-(1-methylpiperidin-4-yl)-2-(5-{[(1H-pyrazol-4-yl)amino]methyl}-1,3,4-thiadiazol- |
| | 2-yl)-1-(2,2,2-trifluoroethyl)-1H-indol-4-amine |
| 2326. | N-((5-(4-(((3S,4R)-3-fluoro-1-methylpiperidin-4-yl)amino)-1-(2,2,2-trifluoroethyl)- |
| | 1H-indol-2-yl)-1,3,4-thiadiazol-2-yl)methyl)cyclopentanecarboxamide |
| 2327. | N-((3S,4R)-3-fluoro-1-methylpiperidin-4-yl)-2-(5-(((2-methoxy-4- |
| | (methylsulfonyl)phenyl)amino)methyl)-1,3,4-thiadiazol-2-yl)-1-(2,2,2-trifluoroethyl)- |
| | 1H-indol-4-amine |
| 2328. | N-((5-(4-(((3S,4R)-3-fluoro-1-methylpiperidin-4-yl)amino)-1-(2,2,2-trifluoroethyl)- |
| | 1H-indol-2-yl)-1,3,4-thiadiazol-2-yl)methyl)benzamide |
| 2329. | N-((5-(4-(((3S,4R)-3-fluoro-1-methylpiperidin-4-yl)amino)-1-(2,2,2-trifluoroethyl)- |
| | 1H-indol-2-yl)-1,3,4-thiadiazol-2-yl)methyl)cyclopropanecarboxamide |
| 2330. | 2-(5-((dimethylamino)methyl)-1,3,4-thiadiazol-2-yl)-N-((3S,4R)-3-fluoro-1- |
| | methylpiperidin-4-yl)-1-(2,2,2-trifluoroethyl)-1H-indol-4-amine |
| 2331. | N-((3S,4S)-3-fluoro-1-methylpiperidin-4-yl)-2-(5-(((2-methoxy-4- |
| | (methylsulfonyl)phenyl)amino)methyl)-1,3,4-thiadiazol-2-yl)-1-(2,2,2-trifluoroethyl)- |
| | 1H-indol-4-amine |
| 2332. | N-{[2-(4-{[(3S,4R)-3-fluoro-1-methylpiperidin-4-yl]amino}-1-(2,2,2-trifluoroethyl)- |
| | 1H-indol-2-yl)-1,3-thiazol-4-yl]methyl}cyclopropanecarboxamide |
| 2333. | N-{[2-(4-{[(3S,4R)-3-fluoro-1-methylpiperidin-4-yl]amino}-1-(2,2,2-trifluoroethyl)- |
| | 1H-indol-2-yl)-1,3-thiazol-4-yl]methyl}benzamide |
| 2334. | (+/−)-N-{[2-(4-{[(3R,4S)-3-fluoro-1-methylpiperidin-4-yl] amino}-1-(2,2,2- |
| | trifluoroethyl)-1H-indol-2-yl)-1,3-thiazol-5-yl]methyl}benzamide |
| 2335. | N-[(2-{4-[(1-methylpiperidin-4-yl)amino]-1-(2,2,2-trifluoroethyl)-1H-indol-2-yl}-1,3- |
| | thiazol-5-yl)methyl]benzamide |
| 2336. | (+/−)-N-[(3R,4S)-3-fluoro-1-methylpiperidin-4-yl]-2-(1,3-thiazol-2-yl)-1-(2,2,2- |
| | trifluoroethyl)-1H-indol-4-amine |
| 2337. | N-[(2-{4-[(1-methylpiperidin-4-yl)amino]-1-(2,2,2-trifluoroethyl)-1H-indol-2-yl}-1,3- |
| | thiazol-5-yl)methyl]cyclopropanecarboxamide |
| 2338. | (+/−)-N-{[2-(4-{[(3R,4S)-3-fluoro-1-methylpiperidin-4-yl]amino}-1-(2,2,2- |
| | trifluoroethyl)-1H-indol-2-yl)-1,3-thiazol-5-yl]methyl}cyclopropanecarboxamide |
| 2339. | (+/−)-2-[5-(aminomethyl)-1,3-thiazol-2-yl]-N-[(3R,4S)-3-fluoro-1-methylpiperidin-4- |
| | yl]-1-(2,2,2-trifluoroethyl)-1H-indol-4-amine |
| 2340. | 2-(4-(aminomethyl)thiazol-2-yl)-N-((3S,4R)-3-fluoro-1-methylpiperidin-4-yl)-1- |
| | (2,2,2-trifluoroethyl)-1H-indol-4-amine |
| 2341. | (+/−)-N-[(3R,4S)-3-fluoro-1-methylpiperidin-4-yl]-2-{5-[(phenylamino)methyl]-1,3- |
| | thiazol-2-yl}-1-(2,2,2-trifluoroethyl)-1H-indol-4-amine |
| 2342. | N-[(3S,4R)-3-fluoro-1-methylpiperidin-4-yl]-2-(5-{[(4-methanesulfonyl-2- |
| | methoxyphenyl)amino]methyl}-1,3-thiazol-2-yl)-1-(2,2,2-trifluoroethyl)-1H-indol-4- |
| | amine |
| 2343. | 2-(5-{[(4-methanesulfonylphenyl)amino]methyl}thiophen-2-yl)-N-(1- |
| | methylpiperidin-4-yl)-1-(2,2,2-trifluoroethyl)-1H-indol-4-amine |
| 2344. | (+/−)-N-[(3R,4S)-3-fluoro-1-methylpiperidin-4-yl]-2-(5-{[(4-methanesulfonyl-2- |
| | methoxyphenyl)amino]methyl}thiophen-2-yl)-1-(2,2,2-trifluoroethyl)-1H-indol-4- |
| | amine |
| 2345. | (+/−)-N-{[5-(4-{[(3R,4S)-3-fluoro-1-methylpiperidin-4-yl]amino}-1-(2,2,2- |
| | trifluoroethyl)-1H-indol-2-yl)thiophen-2-yl]methyl}benzamide |
| 2346. | (+/−)-N-{[5-(4-{[(3R,4S)-3-fluoro-1-methylpiperidin-4-yl]amino}-1-(2,2,2- |
| | trifluoroethyl)-1H-indol-2-yl)thiophen-2-yl]methyl}cyclopropanecarboxamide |
| 2347. | N-(1-methylpiperidin-4-yl)-2-(5-(((4-(methylsulfonyl)phenyl)amino)methyl)thiophen- |
| | 2-yl)-1-(2,2,2-trifluoroethyl)-1H-indol-4-amine |
| 2348. | N-((5-(4-(((3S,4R)-3-fluoro-1-methylpiperidin-4-yl)amino)-1-(2,2,2-trifluoroethyl)- |
| | 1H-indol-2-yl)thiophen-2-yl)methyl)benzamide |
| 2349. | N-((3S,4R)-3-fluoro-1-methylpiperidin-4-yl)-2-(5-(((2-methoxy-4- |
| | (methylsulfonyl)phenyl)amino)methyl)thiophen-2-yl)-1-(2,2,2-trifluoroethyl)-1H- |
| | indol-4-amine |
| 2350. | 2-(5-(amino(cyclohexyl)methyl)-1,3,4-oxadiazol-2-yl)-N-(1-methylpiperidin-4-yl)-1- |
| | (2,2,2-trifluoroethyl)-1H-indol-4-amine |
| 2351. | N-(1-methylpiperidin-4-yl)-2-{5-[(phenylamino)methyl]-1,3,4-oxadiazol-2-yl}-1- |
| | (2,2,2-trifluoroethyl)-1H-indol-4-amine |
| 2352. | 2-(5-(amino(tetrahydro-2H-pyran-4-yl)methyl)-1,3,4-oxadiazol-2-yl)-N-(1- |
| | methylpiperidin-4-yl)-1-(2,2,2-trifluoroethyl)-1H-indol-4-amine |
| 2353. | N-[(5-{4-[(1-methylpiperidin-4-yl)amino]-1-(2,2,2-trifluoroethyl)-1H-indol-2-yl}- |
| | 1,3,4-oxadiazol-2-yl)methyl]cyclopropanecarboxamide |
| 2354. | 2-(5-{[(4-methanesulfonylphenyl)amino]methyl}-1,3,4-oxadiazol-2-yl)-N-(1- |
| | methylpiperidin-4-yl)-1-(2,2,2-trifluoroethyl)-1H-indol-4-amine |
| 2355. | N-(1-methylpiperidin-4-yl)-2-(5-{[(1H-pyrazol-4-yl)amino]methyl}-1,3,4-oxadiazol- |
| | 2-yl)-1-(2,2,2-trifluoroethyl)-1H-indol-4-amine |
| 2356. | 2-(5-(aminomethyl)-1,3,4-oxadiazol-2-yl)-N-(1-methylpiperidin-4-yl)-1-(2,2,2- |
| | trifluoroethyl)-1H-indol-4-amine |
| 2357. | (+/−)-N-[(3R,4S)-3-fluoro-1-methylpiperidin-4-yl]-2-(5-{[(4-methanesulfonyl-2- |
| | methoxyphenyl)amino]methyl}-1,3,4-oxadiazol-2-yl)-1-(2,2,2-trifluoroethyl)-1H- |
| | indol-4-amine |
| 2358. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-2-methoxybenzamide |
| 2359. | (+/−)-2-[5-(aminomethyl)-1,3,4-oxadiazol-2-yl]-N-[(3R,4S)-3-fluoro-1- |
| | methylpiperidin-4-yl]-1-(2,2,2-trifluoroethyl)-1H-indol-4-amine |
| 2360. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-4-methoxybenzamide |
| 2361. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}benzamide |
| 2362. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-3-methoxybenzamide |
| 2363. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}thiophene-2-carboxamide |
| 2364. | (+/−)-2-(5-{[(cyclopropylmethyl)amino]methyl}-1,3,4-oxadiazol-2-yl)-N-[(3R,4S)-3- |
| | fluoro-1-methylpiperidin-4-yl]-1-(2,2,2-trifluoroethyl)-1H-indol-4-amine |
| 2365. | (+/−)-N-[(3R,4S)-3-fluoro-1-methylpiperidin-4-yl]-2-(5-{[(3- |
| | methanesulfonylphenyl)amino]methyl}-1,3,4-oxadiazol-2-yl)-1-(2,2,2-trifluoroethyl)- |
| | 1H-indol-4-amine |
| 2366. | (+/−)-N-[(3R,4R)-3-fluoro-1-methylpiperidin-4-yl]-2-(5-{[(4-methanesulfonyl-2- |
| | methoxyphenyl)amino]methyl}-1,3,4-oxadiazol-2-yl)-1-(2,2,2-trifluoroethyl)-1H- |
| | indol-4-amine |
| 2367. | (+/−)-2-(5-((bis(cyclopropylmethyl)amino)methyl)-1,3,4-oxadiazol-2-yl)-N-((3R,4S)- |
| | 3-fluoro-1-methylpiperidin-4-yl)-1-(2,2,2-trifluoroethyl)-1H-indol-4-amine |
| 2368. | (+/−)-N-((3S,4R)-3-fluoro-1-methylpiperidin-4-yl)-2-(5-(((3- |
| | (methylsulfonyl)phenyl)amino)methyl)-1,3,4-oxadiazol-2-yl)-1-(2,2,2-trifluoroethyl)- |
| | 1H-indol-4-amine |
| 2369. | (+/−)-N-((3S,4R)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2-trifluoroethyl)- |
| | 1H-indol-4-amine |
| 2370. | (+/−)-2-(5-(aminomethyl)-1,3,4-oxadiazol-2-yl)-N-((3S,4R)-3-fluoro-1- |
| | methylpiperidin-4-yl)-1-(2,2,2-trifluoroethyl)-1H-indol-4-amine |
| 2371. | (+/−)-N-((3S,4S)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2-trifluoroethyl)- |
| | 1H-indol-4-amine |
| 2372. | N-((5-(4-(((3S,4R)-3-fluoro-1-methylpiperidin-4-yl)amino)-1-(2,2,2-trifluoroethyl)- |
| | 1H-indol-2-yl)-1,3,4-oxadiazol-2-yl)methyl)benzamide |
| 2373. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)thiophene-2-carboxamide |
| 2374. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-2-methoxybenzamide |
| 2375. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-3-methoxybenzamide |
| 2376. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-4-methoxybenzamide |
| 2377. | (+/−)-2-(5-(((cyclopropylmethyl)amino)methyl)-1,3,4-oxadiazol-2-yl)-N-((3S,4R)-3- |
| | fluoro-1-methylpiperidin-4-yl)-1-(2,2,2-trifluoroethyl)-1H-indol-4-amine |
| 2378. | (+/−)-N-((3R,4R)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2-trifluoroethyl)- |
| | 1H-indol-4-amine |
| 2379. | 2-(3-{[(4-methanesulfonylphenyl)amino]methyl}-1,2,4-oxadiazol-5-yl)-N-(1- |
| | methylpiperidin-4-yl)-1-(2,2,2-trifluoroethyl)-1H-indol-4-amine |
| 2380. | N-[(5-{4-[(1-methylpiperidin-4-yl)amino]-1-(2,2,2-trifluoroethyl)-1H-indol-2-yl}- |
| | 1,2,4-oxadiazol-3-yl)methyl]cyclopropanecarboxamide |
| 2381. | N-[(5-{4-[(1-methylpiperidin-4-yl)amino]-1-(2,2,2-trifluoroethyl)-1H-indol-2-yl}- |
| | 1,2,4-oxadiazol-3-yl)methyl]benzamide |
| 2382. | N-[(5-{4-[(1-methylpiperidin-4-yl)amino]-1-(2,2,2-trifluoroethyl)-1H-indol-2-yl}- |
| | 1,2,4-oxadiazol-3-yl)methyl]thiophene-2-carboxamide |
| 2383. | 1-methyl-N-[(5-{4-[(1-methylpiperidin-4-yl)amino]-1-(2,2,2-trifluoroethyl)-1H-indol- |
| | 2-yl}-1,2,4-oxadiazol-3-yl)methyl]-1H-pyrazole-4-carboxamide |
| 2384. | 1-methyl-N-[(5-{4-[(1-methylpiperidin-4-yl)amino]-1-(2,2,2-trifluoroethyl)-1H-indol- |
| | 2-yl}-1,2,4-oxadiazol-3-yl)methyl]-1H-pyrazole-3-carboxamide |
| 2385. | N-(1-methylpiperidin-4-yl)-2-{5-[(phenylamino)methyl]-1,2,4-oxadiazol-3-yl}-1- |
| | (2,2,2-trifluoroethyl)-1H-indol-4-amine |
| 2386. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]thiophene-2-carboxamide |
| 2387. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]benzamide |
| 2388. | (+/−)-(1S,2R)-2-fluoro-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1- |
| | carboxamide |
| 2389. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]cyclopropanecarboxamide |
| 2390. | (+/−)-(1S,2S)-2-fluoro-N-[(3-{4-[(1-methylpiperidin-4-yl)amino]-1-(2,2,2- |
| | trifluoroethyl)-1H-indol-2-yl}-1,2,4-oxadiazol-5-yl]methyl]cyclopropane-1- |
| | carboxamide |
| 2391. | 4-chloro-N-[(3-{4-[(1-methylpiperidin-4-yl)amino]-1-(2,2,2-trifluoroethyl)-1H-indol- |
| | 2-yl}-1,2,4-oxadiazol-5-yl)methyl]benzamide |
| 2392. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]-1,3-thiazole-2-carboxamide |
| 2393. | 4-fluoro-N-[(3-{4-[(1-methylpiperidin-4-yl)amino]-1-(2,2,2-trifluoroethyl)-1H-indol- |
| | 2-yl}-1,2,4-oxadiazol-5-yl)methyl]benzamide |
| 2394. | 4-cyano-N-[(3-{4-[(1-methylpiperidin-4-yl)amino]-1-(2,2,2-trifluoroethyl)-1H-indol- |
| | 2-yl}-1,2,4-oxadiazol-5-yl)methyl]benzamide |
| 2395. | 1-methyl-N-[(3-{4-[(1-methylpiperidin-4-yl)amino]-1-(2,2,2-trifluoroethyl)-1H-indol- |
| | 2-yl}-1,2,4-oxadiazol-5-yl)methyl]-1H-pyrrole-3-carboxamide |
| 2396. | 3-[(3-{4-[(1-methylpiperidin-4-yl)amino]-1-(2,2,2-trifluoroethyl)-1H-indol-2-yl}- |
| | 1,2,4-oxadiazol-5-yl)methyl]-1-phenylurea |
| 2397. | 1-methyl-N-[(3-{4-[(1-methylpiperidin-4-yl)amino]-1-(2,2,2-trifluoroethyl)-1H-indol- |
| | 2-yl}-1,2,4-oxadiazol-5-yl)methyl]-1H-pyrazole-4-carboxamide |
| 2398. | 1-methyl-N-[(3-{4-[(1-methylpiperidin-4-yl)amino]-1-(2,2,2-trifluoroethyl)-1H-indol- |
| | 2-yl}-1,2,4-oxadiazol-5-yl)methyl]-1H-pyrazole-3-carboxamide |
| 2399. | (+/−)-(1R,2R)-N-[(3-{4-[(1-methylpiperidin-4-yl)amino]-1-(2,2,2-trifluoroethyl)-1H- |
| | indol-2-yl}-1,2,4-oxadiazol-5-yl)methyl]-2-phenylcyclopropane-1-carboxamide |
| 2400. | (+/−)-(1R,2R)-2-methyl-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1-carboxamide |
| 2401. | (+/−)-(1R,2S)-2-methyl-N-[(3-{4-[(1-methylpiperidin-4-yl)amino]-1-(2,2,2- |
| | trifluoroethyl)-1H-indol-2-yl}-1,2,4-oxadiazol-5-yljmethyl]cyclopropane-1- |
| | carboxamide |
| 2402. | N-[(3-{4-[(oxan-4-yl)amino]-1-(2,2,2-trifluoroethyl)-1H-indol-2-yl}-1,2,4-oxadiazol- |
| | 5-yl)methyl]cyclopropanecarboxamide |
| 2403. | N-({3-[4-(benzylamino)-1-(2,2,2-trifluoroethyl)-1H-indol-2-yl]-1,2,4-oxadiazol-5- |
| | yl}methyl)cyclopropanecarboxamide |
| 2404. | N-[(3-{4-[(1-acetylpiperidin-4-yl)amino]-1-(2,2,2-trifluoroethyl)-1H-indol-2-yl}- |
| | 1,2,4-oxadiazol-5-yl)methyl]cyclopropanecarboxamide |
| 2405. | N-[(3-{4-[(1,1-dioxo-1λ⁶-thian-4-yl)amino]-1-(2,2,2-trifluoroethyl)-1H-indol-2-yl}- |
| | 1,2,4-oxadiazol-5-yl)methyl]cyclopropanecarboxamide |
| 2406. | N-[(3-{4-[(1-benzylpiperidin-4-yl)amino]-1-(2,2,2-trifluoroethyl)-1H-indol-2-yl}- |
| | 1,2,4-oxadiazol-5-yl)methyl]cyclopropanecarboxamide |
| 2407. | N-[(3-{4-[(1-cyclopropylpiperidin-4-yl)amino]-1-(2,2,2-trifluoroethyl)-1H-indol-2- |
| | yl}-1,2,4-oxadiazol-5-yl)methyl]cyclopropanecarboxamide |
| 2408. | N-[(3-{4-[(cyclopropylmethyl)amino]-1-(2,2,2-trifluoroethyl)-1H-indol-2-yl}-1,2,4- |
| | oxadiazol-5-yl)methyl]cyclopropanecarboxamide |
| 2409. | N-[(3-{4-[(cyclobutylmethyl)amino]-1-(2,2,2-trifluoroethyl)-1H-indol-2-yl}-1,2,4- |
| | oxadiazol-5-yl)methyl]cyclopropanecarboxamide |
| 2410. | (+/−)-N-[(3-{4-[(pyrrolidin-3-yl)amino]-1-(2,2,2-trifluoroethyl)-1H-indol-2-yl}-1,2,4- |
| | oxadiazol-5-yl)methyl]cyclopropanecarboxamide |
| 2411. | N-[(3-{4-[(1-ethylpiperidin-4-yl)amino]-1-(2,2,2-trifluoroethyl)-1H-indol-2-yl}-1,2,4- |
| | oxadiazol-5-yl)methyl]cyclopropanecarboxamide |
| 2412. | (+/−)-N-[(3-{4-[(1-methylpyrrolidin-3-yl)amino]-1-(2,2,2-trifluoroethyl)-1H-indol-2- |
| | yl}-1,2,4-oxadiazol-5-yl)methyl]cyclopropanecarboxamide |
| 2413. | N-{[3-(4-{[(273zetidine-3-yl)methyl]amino}-1-(2,2,2-trifluoroethyl)-1H-indol-2-yl)- |
| | 1,2,4-oxadiazol-5-yl]methyl}cyclopropanecarboxamide |
| 2414. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}cyclopropanecarboxamide |
| 2415. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}benzamide |
| 2416. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}cyclopropanecarboxamide |
| 2417. | N-{[3-(4-{[(3R,4S)-3-fluoro-1-methylpiperidin-4-yl]amino}-1-(2,2,2-trifluoroethyl)- |
| | 1H-indol-2-yl)-1,2,4-oxadiazol-5-yl]methyl}cyclopropanecarboxamide |
| 2418. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-2-carboxamide |
| 2419. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3-carboxamide |
| 2420. | (+/−)-N-[(3R,4S)-3-fluoro-1-methylpiperidin-4-yl]-2-(5-{[(4- |
| | methanesulfonylphenyl)amino]methyl}-1,2,4-oxadiazol-3-yl)-1-(2,2,2-trifluoroethyl)- |
| | 1H-indol-4-amine |
| 2421. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1,3-thiazole-5- |
| | carboxamide |
| 2422. | (+/−)-1-ethyl-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2423. | (+/−)-(1R,2R)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-2-phenylcyclopropane-1- |
| | carboxamide |
| 2424. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-pyrrole-3-carboxamide |
| 2425. | N-{[3-(4-{[(3R,4S)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-pyrrole-3-carboxamide |
| 2426. | (+/−)-N-[(3R,4S)-3-fluoro-1-methylpiperidin-4-yl]-2-(5-{[(4-methanesulfonyl-2- |
| | methoxyphenyl)amino]methyl}-1,2,4-oxadiazol-3-yl)-1-(2,2,2-trifluoroethyl)-1H- |
| | indol-4-amine |
| 2427. | (+/−)-1-tert-butyl-N-{[3-(4-{[(3R,4S)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole-3- |
| | carboxamide |
| 2428. | (+/−)-(1R,2R)-N-{[3-(4-{[(3RS,4SR)-3-fluoro-1-methylpiperidin-4-yl]amino}-1- |
| | (2,2,2-trifluoroethyl)-1H-indol-2-yl)-1,2,4-oxadiazol-5-yl]methyl}-2- |
| | phenylcyclopropane-1-carboxamide |
| 2429. | 1-ethyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2430. | 1-ethyl-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2431. | (+/−)-N-[(3R,4S)-3-fluoropiperidin-4-yl]-2-(5-{[(4-methanesulfonyl-2- |
| | methoxyphenyl)amino]methyl}-1,2,4-oxadiazol-3-yl)-1-(2,2,2-trifluoroethyl)-1H- |
| | indol-4-amine |
| 2432. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}thiophene-3-carboxamide |
| 2433. | N-{[3-(4-{[(3R,4S)-3-fluoro-1-methylpiperidin-4-yl]amino}-1-(2,2,2-trifluoroethyl)- |
| | 1H-indol-2-yl)-1,2,4-oxadiazol-5-yl]methyl}thiophene-3-carboxamide |
| 2434. | (+/−)-N-[(3R,4S)-3-fluoropiperidin-4-yl]-2-(5-{[methyl(1H-pyrazol-4- |
| | yl)amino]methyl}-1,2,4-oxadiazol-3-yl)-1-(2,2,2-trifluoroethyl)-1H-indol-4-amine |
| 2435. | (+/−)-N-[(3R,4S)-3-fluoro-1-methylpiperidin-4-yl]-2-(5-{[methyl(1H-pyrazol-4- |
| | yl)amino]methyl}-1,2,4-oxadiazol-3-yl)-1-(2,2,2-trifluoroethyl)-1H-indol-4-amine |
| 2436. | (1RS,2RS)-2-cyano-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}cyclopropane-1- |
| | carboxamide |
| 2437. | 1-tert-butyl-N-{[3-(4-{[(3 S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2438. | (1RS,2SR)-2-cyano-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}cyclopropane-1- |
| | carboxamide |
| 2439. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}indolizine-2-carboxamide |
| 2440. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-phenyl-1H-imidazole-4-carboxamide |
| 2441. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-pyrrole-3- |
| | carboxamide |
| 2442. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-pyrrole-3- |
| | carboxamide |
| 2443. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyridine-4-carboxamide |
| 2444. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyridine-2-carboxamide |
| 2445. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyridine-3-carboxamide |
| 2446. | N-[(3S,4R)-3-fluoropiperidin-4-yl]-2-(5-{[(1H-pyrazol-4-yl)amino]methyl}-1,2,4- |
| | oxadiazol-3-yl)-1-(2,2,2-trifluoroethyl)-1H-indol-4-amine |
| 2447. | benzyl N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamate |
| 2448. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-fluoroethyl)-1H-pyrrole-3- |
| | carboxamide |
| 2449. | (1S,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-phenylcyclopropane-1- |
| | carboxamide |
| 2450. | (1R,2S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-phenylcyclopropane-1- |
| | carboxamide |
| 2451. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-5-methylthiophene-3-carboxamide |
| 2452. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-4-methylthiophene-3-carboxamide |
| 2453. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(4- |
| | fluorophenyl)cyclopropane-1-carboxamide |
| 2454. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-2-methylthiophene-3-carboxamide |
| 2455. | (1s,3r)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-methylcyclobutane-1- |
| | carboxamide |
| 2456. | 5-chloro-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3-carboxamide |
| 2457. | N-[(3S,4R)-3-fluoropiperidin-4-yl]-2-(5-{[(1H-pyrazol-5-yl)amino]methyl}-1,2,4- |
| | oxadiazol-3-yl)-1-(2,2,2-trifluoroethyl)-1H-indol-4-amine |
| 2458. | 2-chloro-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3-carboxamide |
| 2459. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyrazolo[1,5-a]pyridine-2-carboxamide |
| 2460. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}imidazo[1,2-a]pyridine-2-carboxamide |
| 2461. | 1-cyclopropyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2462. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1,5-dimethyl-1H-pyrrole-3-carboxamide |
| 2463. | 4-(dimethylamino)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}benzamide |
| 2464. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}benzenesulfonamide |
| 2465. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}cyclobutanecarboxamide |
| 2466. | (1r,3s)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-methylcyclobutane-1- |
| | carboxamide |
| 2467. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(2- |
| | fluorophenyl)cyclopropane-1-carboxamide |
| 2468. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(1-methyl-1H-pyrazol- |
| | 4-yl)cyclopropane-1-carboxamide |
| 2469. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(pyridin-2- |
| | yl)cyclopropane-1-carboxamide |
| 2470. | N-[(3S,4R)-3-fluoro-1-methylpiperidin-4-yl]-2-(5-{[(1H-pyrazol-4-yl)amino]methyl}- |
| | 1,2,4-oxadiazol-3-yl)-1-(2,2,2-trifluoroethyl)-1H-indol-4-amine |
| 2471. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}acetamide |
| 2472. | 1-[2-(dimethylamino)ethyl]-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5-yl]methyl}-1H- |
| | pyrrole-3-carboxamide |
| 2473. | N-[(3S,4R)-3-fluoro-1-methylpiperidin-4-yl]-2-(5-{[(1H-pyrazol-5-yl)amino]methyl}- |
| | 1,2,4-oxadiazol-3-yl)-1-(2,2,2-trifluoroethyl)-1H-indol-4-amine |
| 2474. | 1-tert-butyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrazole-4- |
| | carboxamide |
| 2475. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-imidazole-4- |
| | carboxamide |
| 2476. | (1S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2,2- |
| | dimethylcyclopropane-1-carboxamide |
| 2477. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(4-methylpiperazin-1- |
| | yl)methyl]cyclopropane-1-carboxamide |
| 2478. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(3- |
| | fluorophenyl)cyclopropane-1-carboxamide |
| 2479. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-12,3-triazole-4- |
| | carboxamide |
| 2480. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(1-hydroxypropan-2-yl)-1H-pyrrole-3- |
| | carboxamide |
| 2481. | 2-[3-({[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamoyl)-1H-pyrrol-1- |
| | yl]acetic acid |
| 2482. | (1R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2,2- |
| | dimethylcyclopropane-1-carboxamide |
| 2483. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methylpropyl)-1H-pyrrole-3- |
| | carboxamide |
| 2484. | 1-(cyclopropylmethyl)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole-3- |
| | carboxamide |
| 2485. | 3-(dimethylamino)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}benzamide |
| 2486. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-4-(pyrrolidin-1-yl)benzamide |
| 2487. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-hydroxypropyl)-1H-pyrrole-3- |
| | carboxamide |
| 2488. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(morpholin-4- |
| | yl)methyl]cyclopropane-1-carboxamide |
| 2489. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-{[(propan-2- |
| | yl)amino]methyl}cyclopropane-1-carboxamide |
| 2490. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2- |
| | [(propylamino)methyl]cyclopropane-1-carboxamide |
| 2491. | 3-[3-({[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamoyl)-1H-pyrrol-1- |
| | yl]propanoic acid |
| 2492. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-hydroxyethyl)-1H-pyrrole-3- |
| | carboxamide |
| 2493. | 1-(2,2-difluoroethyl)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole-3- |
| | carboxamide |
| 2494. | N-{[3-(4-{[(3S,4R)-3-fluoro-1-methylpiperidin-4-yl] amino}-1-(2,2,2-trifluoroethyl)- |
| | 1H-indol-2-yl)-1,2,4-oxadiazol-5-yl]methyl}-1-(2-methoxy-2-methylpropyl)-1H- |
| | pyrrole-3-carboxamide |
| 2495. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-hydroxy-2-methylpropyl)-1H- |
| | pyrrole-3-carboxamide |
| 2496. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methoxypropyl)-1H-pyrrole-3- |
| | carboxamide |
| 2497. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-indole-6-carboxamide |
| 2498. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-pyrazole-4- |
| | carboxamide |
| 2499. | 4-(4,4-difluoropiperidin-1-yl)-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}benzamide |
| 2500. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-imidazole-5- |
| | carboxamide |
| 2501. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1H-indole-5-carboxamide |
| 2502. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-indole-5-carboxamide |
| 2503. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-4-(morpholin-4-yl)benzamide |
| 2504. | 2-[5-(aminomethyl)-1,2,4-oxadiazol-3-yl]-N-[(3S,4R)-3-fluoro-1-methylpiperidin-4- |
| | yl]-1-(2,2,2-trifluoroethyl)-1H-indol-4-amine |
| 2505. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-(morpholin-4-yl)benzamide |
| 2506. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-(pyrrolidin-1-yl)benzamide |
| 2507. | (1R,2R)-2-[(dimethylamino)methyl]-N-{[3-(4-{[(3S,4R)-3-fluoro-1-methylpiperidin- |
| | 4-yl]amino}-1-(2,2,2-trifluoroethyl)-1H-indol-2-yl)-1,2,4-oxadiazol-5- |
| | yl]methyl}cyclopropane-1-carboxamide |
| 2508. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(pyrrolidin-1- |
| | yl)methyl]cyclopropane-1-carboxamide |
| 2509. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-methyl-1H-indole-5-carboxamide |
| 2510. | N-{[3-(4-{[(3S,4R)-3-fluoro-1-methylpiperidin-4-yl] amino}-1-(2,2,2-trifluoroethyl)- |
| | 1H-indol-2-yl)-1,2,4-oxadiazol-5-yl]methyl}-1H-indole-6-carboxamide |
| 2511. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-imidazole-5- |
| | carboxamide |
| 2512. | (1R,2R)-2-ethyl-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}cyclopropane-1- |
| | carboxamide |
| 2513. | 1-tert-butyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-imidazole-5- |
| | carboxamide |
| 2514. | (1S,2S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-{[(propan-2- |
| | yl)amino]methyl}cyclopropane-1-carboxamide |
| 2515. | N-{[3-(4-{[(3S,4R)-1-ethyl-3-fluoropiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-pyrrole-3- |
| | carboxamide |
| 2516. | N-{[3-(4-{[(3R,4S)-1-ethyl-3-fluoropiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-pyrrole-3- |
| | carboxamide |
| 2517. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-methyl-1H-indole-6-carboxamide |
| 2518. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}cyclopropanecarboxamide |
| 2519. | (+/−)-N-{[5-(4-{[(3R,4S)-3-fluoro-1-methylpiperidin-4-yl] amino}-1-(2,2,2- |
| | trifluoroethyl)-1H-indol-2-yl)-1,2,4-oxadiazol-3-yl]methyl}benzamide |
| 2520. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}thiophene-2-carboxamide |
| 2521. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}thiophene-3-carboxamide |
| 2522. | (+/−)-N-{[5-(4-{[(3R,4S)-3-fluoro-1-methylpiperidin-4-yl] amino}-1-(2,2,2- |
| | trifluoroethyl)-1H-indol-2-yl)-1,2,4-oxadiazol-3-yl]methyl}-1,3-thiazole-5- |
| | carboxamide |
| 2523. | N-((3-(4-(((3S,4R)-3-fluoro-1-methylpiperidin-4-yl)amino)-1-(2,2,2-trifluoroethyl)- |
| | 1H-indol-2-yl)-1,2,4-oxadiazol-5-yl)methyl)-1-(1-fluoropropan-2-yl)-1H-pyrrole-3- |
| | carboxamide |
| 2524. | N-((3-(4-(((3S,4R)-1-ethyl-3-fluoropiperidin-4-yl)amino)-1-(2,2,2-trifluoroethyl)-1H- |
| | indol-2-yl)-1,2,4-oxadiazol-5-yl)methyl)-1-(2-fluoroethyl)-1H-pyrrole-3-carboxamide |
| 2525. | N-((3-(4-(((3S,4R)-3-fluoro-1-methylpiperidin-4-yl)amino)-1-(2,2,2-trifluoroethyl)- |
| | 1H-indol-2-yl)-1,2,4-oxadiazol-5-yl)methyl)-1-(1-methoxypropan-2-yl)-1H-pyrrole-3- |
| | carboxamide |

### Example 2: Effect of Compound 2 on Cellular Proliferation in Human Cell Lines

Compound 2 is an indole compound substituted with a trifluoroethyl group at the 1-position; propynyl amino-methoxy-methylsulfonyl phenyl group at the 2-position; and a heterocycle-substituted amino group at the 4-position.

The effect of Compound 2 on cellular proliferation was evaluated in nineteen human cell lines. Seven of the cell lines contained homozygous Y220C mutant p53, three of the cell lines contained heterozygous Y220C mutant p53 with an additional p53 mutation on a second allele, six of the cell lines contained other mutant p53 including R175H, G245S, R248Q, R273H, R273C and R282W, two of the cell lines contained wild type p53, and one cell line had the p53 gene deleted via CRISPR technology. Additionally, an MTT assay was used to assess the activity of Compound 2 across five mouse cell lines harboring Humanized p53 knock-in (Hupki)-p53 Y220C mutant.

Cell lines and reagents: Source of human cell lines, histological subtypes, TP53 status, growth conditions, and 5 day MTT seeding densities for 96-well plate are listed in TABLE 4. The mouse HUPKI-p53 Y220C primary cells lines were generated from tumors that arose in HUPKI-p53 Y220C mutant mice. Mouse HUPKI-p53 Y220C cell line information is shown in TABLE 5.

TABLE 4

| Cell Line | Cell | Cell Line | p53 | Growth | Seeding |
|---|---|---|---|---|---|
| Name | source | Type | status | medium | density |
| HCC2935 | ATCC | lung adenocarcinoma | Y220C | RPMI1640 + 10% | 3000 |
| | | | | FBS | |
| SNU-NCC-19 | KCLB | intestinal adenocarcinoma | Y220C | RPMI1640 + 10% | 4000 |
| | | | | FBS | |
| BxPC-3 | ATCC | pancreatic | Y220C | RPMI1640 + 10% | 2000 |
| | | adenocarcinoma | | FBS | |
| NUGC-3 | JCRB | gastric adenocarcinoma | Y220C | RPMI1640 + 10% | 1500 |
| | | | | FBS | |
| MFE-296 | DSMZ | endometrial | Y220C′, | MEM + 10% FBS | 1500 |
| | | adenocarcinoma | R306*′ | | |
| HUH-7 | JCRB | hepato cellular carcinoma | Y220C | DMEM + 10% FBS | 3000 |
| KON | JCRB | oral squamous carcinoma | Y220C | DMEM + 10% FBS | 2000 |
| T3M-4 | RIKEN | pancreatic | Y220C | DMEM-F12 HAM + | 750 |
| | | adenocarcinoma | | 10% FBS | |
| HCC1419 | ATCC | breast ductal carcinoma | Y220C′, | RPMI1640 + 10% | 3000 |
| | | | A74fs*47′ | FBS | |
| TE-8 | RIKEN | Esophageal carcinoma | Y220C, M237I | RPMI1640 + 10% | 3000 |
| | | | | FBS | |
| TOV112D | ATCC | ovarian endometroid | R175H | DMEM + 10% FBS | 1500 |
| | | carcinoma | | | |
| NUGC3_KO | PMV | gastric adenocarcinoma | KO | RPMI1640 + 10% | 2000 |
| | | | | FBS | |
| EFE184 | DSMZ | endometrial carcinoma | R282W | RPMI1640 + 10% | 1000 |
| | | | | FBS | |
| HC116 | Sigma | colorectal carcinoma | WT | Mccoy's 5A + 10% | 750 |
| | | | | FBS | |
| SF295 | AddexBio | glioblastoma | R248Q | RPMI1640 + 10% | 1000 |
| | | | | FBS | |
| A431 | ATCC | epidermoid carcinoma | R273H | DMEM + 10% FBS | 1500 |
| C33A | ATCC | retinoblastoma | R273C | EMEM + 10% FBS | 2000 |
| SU.86.86 | ATCC | pancreatic carcinoma | G245S, G360V | RPMI1640 + 10% | 1500 |
| | | | | FBS | |
| SJSA-1 | ATCC | osteosarcoma | WT | RPMI1640 + 10% | 2500 |
| | | | | FBS | |

′indicates heterozygous; otherwise homozygous
fs, frameshift
*stop

TABLE 5

| Cell Line | Cell Line | p53 | Growth | Seeding |
|---|---|---|---|---|
| Name | Type | status | medium | density |
| MT173 | spindle cell sarcoma | Y220C | DMEM + 10% FBS | 3000 |
| MT245 | sarcoma | Y220C | DMEM + 10% FBS | 1000 |
| MT306 | sarcoma | Y220C | DMEM + 10% FBS | 2000 |
| MT373 | sarcoma | Y220C | DMEM + 10% FBS | 1000 |
| MT379 | angiosarcoma | Y220C | DMEM + 10% FBS | 1000 |

Cellular proliferation assays: Antiproliferative activity of Compound 2 was evaluated using the MTT assay in 96-well plate format. Cell viability was determined by measuring the reduction of 3-(4,5-Dimethylthiazol-2-yl)-2,5 diphenyltetrazolium bromide) (MTT) to formazan. Briefly, cells were seeded at a density of 750˜ 4000 cells per well in 96-well microtiter plates in a volume of 180 μL growth medium. 180 μL of cell free medium was added to wells for MTT background. MTT was dissolved in PBS at 5 mg/mL and stored at 4° C. Compound 2 was dissolved in 100% DMSO at 10 mM and stored at −20° C. Plates were incubated at 37° C. with 5% CO₂ for 24 hours before adding Compound 2. For the human cell line treatment, Compound 2 was tested at 20 μM followed by 7 additional 2-fold serial dilutions, while for mouse cell line treatment the top dose of Compound 2 was 10 μM. Compound 2 was prepared at ten times the final assay concentration in growth medium containing 2% dimethyl sulfoxide (DMSO) on drug dilution plate, 20 μL of appropriate dilution was added to cell culture plate in duplicates. 20 μL of medium containing 2% DMSO was added to the wells for control (CTRL) and MTT background (BK).

Antiproliferative activity of Compound 2 was assessed 5 days later by the addition of MTT. Plates were incubated with 50 μL per well of MTT at 5 mg/ml dissolved in PBS buffer for 2 hours at 37° C. with 5% CO₂. Thereafter, the MTT was gently aspirated out and 50 μL of 100% ethanol was added to each well to dissolve the formazan crystals. The conversion of MTT into formazan by viable cells was measured by microplate reader for absorbance with the wavelength of 570 nm and reference wavelength of 650 nm. The results were presented as a percentage of the viability of untreated cells (control), which were regarded as 100% viable using the formula:

$$percent viability = \frac{Mean absorance of experimental wells}{Mean absorance of control wells} \times 100$$

EC₅₀ values were determined from the regression of a plot of the Logarithm of concentration versus percent of viability by XLfit IDBS. TABLE 6 shows the plate set up for the human cell line cellular proliferation assay. TABLE 7 shows the plate set up for the mouse cell line cellular proliferation assay.

TABLE 6

| Map | 1 | 2 | 3 | 4 | 5 | 6 | 7 | 8 | 9 | 10 | 11 | 12 |
|---|---|---|---|---|---|---|---|---|---|---|---|---|
| A | BK | BK | 20 | 20 | 20 | 20 | 20 | 20 | 20 | 20 | 20 | 20 |
| B | BK | BK | 10 | 10 | 10 | 10 | 10 | 10 | 10 | 10 | 10 | 10 |
| C | CTRL | CTRL | 5 | 5 | 5 | 5 | 5 | 5 | 5 | 5 | 5 | 5 |
| D | CTRL | CTRL | 2.5 | 2.5 | 2.5 | 2.5 | 2.5 | 2.5 | 2.5 | 2.5 | 2.5 | 2.5 |
| E | CTRL | CTRL | 1.25 | 1.25 | 1.25 | 1.25 | 1.25 | 1.25 | 1.25 | 1.25 | 1.25 | 1.25 |
| F | CTRL | CTRL | 0.63 | 0.63 | 0.63 | 0.63 | 0.63 | 0.63 | 0.63 | 0.63 | 0.63 | 0.63 |
| G | CTRL | CTRL | 0.31 | 0.31 | 0.31 | 0.31 | 0.31 | 0.31 | 0.31 | 0.31 | 0.31 | 0.31 |
| H | CTRL | CTRL | 0.16 | 0.16 | 0.16 | 0.16 | 0.16 | 0.16 | 0.16 | 0.16 | 0.16 | 0.16 |
| Compound | | | drug 1 | | drug 2 | | drug 3 | | drug 4 | | drug 5 | |

TABLE 7

| Map | 1 | 2 | 3 | 4 | 5 | 6 | 7 | 8 | 9 | 10 | 11 | 12 |
|---|---|---|---|---|---|---|---|---|---|---|---|---|
| A | BK | BK | 10 | 10 | 10 | 10 | 10 | 10 | 10 | 10 | 10 | 10 |
| B | BK | BK | 5 | 5 | 5 | 5 | 5 | 5 | 5 | 5 | 5 | 5 |
| C | CTRL | CTRL | 2.5 | 2.5 | 2.5 | 2.5 | 2.5 | 2.5 | 2.5 | 2.5 | 2.5 | 2.5 |
| D | CTRL | CTRL | 1.25 | 1.25 | 1.25 | 1.25 | 1.25 | 1.25 | 1.25 | 1.25 | 1.25 | 1.25 |
| E | CTRL | CTRL | 0.63 | 0.63 | 0.63 | 0.63 | 0.63 | 0.63 | 0.63 | 0.63 | 0.63 | 0.63 |
| F | CTRL | CTRL | 0.31 | 0.31 | 0.31 | 0.31 | 0.31 | 0.31 | 0.31 | 0.31 | 0.31 | 0.31 |
| G | CTRL | CTRL | 0.16 | 0.16 | 0.16 | 0.16 | 0.16 | 0.16 | 0.16 | 0.16 | 0.16 | 0.16 |
| H | CTRL | CTRL | 0.08 | 0.08 | 0.08 | 0.08 | 0.08 | 0.08 | 0.08 | 0.08 | 0.08 | 0.08 |
| Compound | | | drug 1 | | drug 2 | | drug 3 | | drug 4 | | drug 5 | |

In cell lines expressing homozygous Y220C mutant p53, Compound 2 inhibited cellular proliferation with EC₅₀ values range from 0.231 μM to 1.806 μM. Of the three human cell lines that contained heterozygous Y220C mutant p53 with additional mutation on a second allele, HCC-1419 and TE-8 showed a modest response to this compound with EC₅₀ values of 4.872 μM and 8.657 μM, while MFE-296 was more sensitive and had an EC₅₀ value at 0.497 μM. Cell lines that contained other mutant p53 including TOV112D (R175H), SU.86.86 (G245S), SF295 (R248Q), A-431 (R273H), C-33A (R273C) and EFE-184 (R282W) showed a reduced response to Compound 2 with EC₅₀ values ranging from 11.189 μM to 18.472 μM. Human cell lines with WT p53, SJSA-1 and HCT116, and a cell line lacking p53, NUGC-3-p53 knock out, showed the least response to Compound 2 with EC₅₀ values ranging from 15.503 μM to above 20 μM, respectively. Compound 2 showed potent and selective antiproliferative activity against a broad spectrum of tumor cell lines bearing Y220C mutant p53.

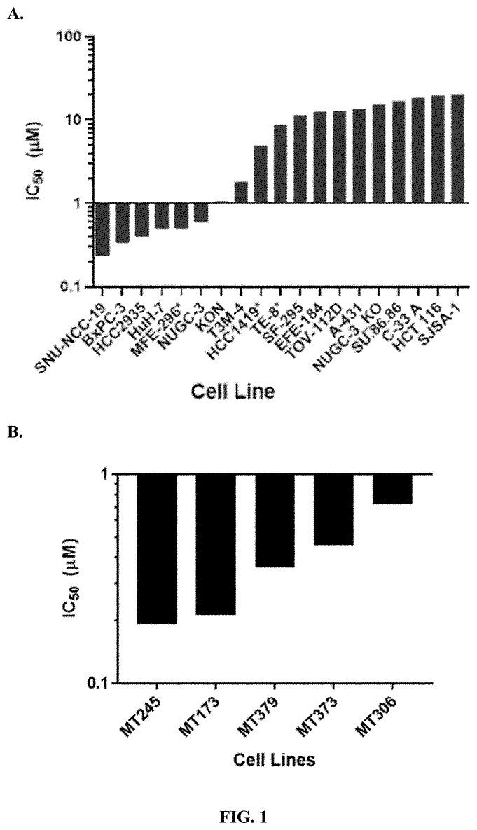

FIG. 1 PANEL A shows IC₅₀ values (μM) of the 5-day MTT assay using Compound 2 in human cell lines. * indicates cell lines with a second, unrelated p53 mutation. TABLE 8 shows activity across cell lines in the MTT assay (IC₅₀, μM) in human cell lines.

TABLE 8

| Cell Line | Cell Line | p53 | MTT IC50 |
|---|---|---|---|
| Name | Type | Status | (μM) |
| SNU-NCC-19 | intestinal adenocarcinoma | Y220C | 0.231 |
| BxPC-3 | pancreatic adenocarcinoma | Y220C | 0.332 |
| HCC2935 | lung adenocarcinoma | Y220C | 0.403 |
| HuH-7 | hepato cellular carcinoma | Y220C | 0.491 |
| MFE-296* | endometrial adenocarcinoma | Y220C, R306* | 0.497 |
| NUGC-3 | gastric adenocarcinoma | Y220C | 0.586 |
| KON | oral squamous carcinoma | Y220C | 1.054 |
| T3M-4 | pancreatic adenocarcinoma | Y220C | 1.806 |
| HCC1419* | breast ductal carcinoma | Y220C, A74fs*47 | 4.872 |
| TE-8* | esophageal carcinoma | Y220C, M237I | 8.657 |
| SF-295 | glioblastoma | R248Q | 11.189 |
| EFE-184 | endometrial carcinoma | R282W | 12.445 |
| TOV-112D | ovarian endometroid carcinoma | R175H | 12.972 |
| A-431 | epidermoid carcinoma | R273H | 13.751 |
| NUGC-3_KO | gastric adenocarcinoma | KO | 15.503 |
| SU.86.86 | pancreatic carcinoma | G245S | 16.798 |
| C-33 A | retinoblastoma | R273C | 18.472 |
| HCT 116 | colorectal carcinoma | WT | 19.269 |
| SJSA-1 | osteosarcoma | WT | >20 |

*Stop;
fs, frameshift

The effect of Compound 2 on cellular proliferation was also evaluated in five mouse Hupki-p53 Y220C mutant cell lines, which are homozygous for the p53 Y220C mutation. Compound 2 treatment showed robust inhibition of cellular proliferation with EC₅₀ values ranging from 0.192 μM to 0.722 μM among the five cell lines. FIG. 1. PANEL B shows IC₅₀ values of the 5-day MTT assay using Compound 2 in mouse cell lines. TABLE 9 shows activity across cell lines in the MTT assay (IC₅₀, μM) in mouse cell lines with humanized p53 Y220C.

TABLE 9

| Cell Line | Cell Line | p53 | MTT IC₅₀ |
|---|---|---|---|
| Name | Type | Status | (μM) |
| MT245 | sarcoma | Y220C | 0.192 |
| MT173 | spindle cell sarcoma | Y220C | 0.212 |
| MT379 | angiosarcoma | Y220C | 0.359 |
| MT373 | sarcoma | Y220C | 0.459 |
| MT306 | sarcoma | Y220C | 0.722 |

### Example 3: Compound 2 Restores the p53 Pathway in Mutant p53 Y220C Cells

Compound 2's ability to restore mutant p53 Y220C protein to p53 wild-type function, e.g. to activate transcription of p53-downstream genes, and Compound 2's potential off-target effects in cells lacking p53 Y220C protein were investigated. The effect of Compound 2 in 12 cell lines carrying the p53 Y220C mutation on mRNA levels of two downstream p53 target genes (CDKN1A (p21) and MDM2) was investigated using RT-qPCR. p21 is the most dynamic p53 responsive gene, and MDM2 is one of the most selective of the p53 responsive genes. The selectivity of Compound 2 was also monitored by assessing activity in cells lacking p53 Y220C, including p53 WT cells, cells harboring p53 mutations other than Y220C, and cells which have had both p53 alleles excised via CRISPR technology. Restoration of the p53-dependent transcription pathway was assessed by profiling the expression of a cassette of 84 p53-related genes following Compound 2 in p53 Y220C containing cells (NUGC-3 and T3M4 cells) compared to NUGC3 knockout cells (KO). Additionally, transcriptional activity of p53 Y220C in Compound 2 treated cells were compared to the transcriptional pattern observed from WT p53 in the same cellular background (NUGC-3 KO with inducible WT p53).

Cells: TABLE 10 shows cell sources, histological subtype, p53 status, and growth medium of the cell lines. NUGC-3KO was generated by knocking out mutant p53 from Intron 1 to Exons 6 using CRISPR technology. RPMI-1640 Medium, DMEM/F-12, 1:1 mixture (D8437), and Heat Inactivated Fetal Bovine Serum (FBS, F8192) were purchased from Sigma-Aldrich. All cells were cultured in the indicated growth medium, supplemented with indicated concentration of FBS in a humidified incubator with 5% CO₂ at 37° C.

TABLE 10

| Cell | p53 | Additional | Cell Line | Growth |
|---|---|---|---|---|
| Line | Mut | p53 Mutation | Type | Medium |
| NUGC-3 | Y220C | | gastric adenocarcinoma | RPMI-1640 + 10% |
| | | | | FBS |
| T3M-4 | Y220C | | pancreatic | DMEM: F12 + 10% |
| | | | adenocarcinoma | FBS |
| HCC1419 | | A74fs*47 | breast ductal carcinoma | RPMI-1640 + 10% |
| | | (Heterozygous) | | FBS |
| SNU-NCC-19 | Y220C | | intestinal | RPMI-1640 + 10% |
| | | | adenocarcinoma | FBS |
| HCC366 | Y220C | | lung adenosquamous | RPMI-1640 + 10% |
| | | | carcinoma | FBS |
| BxPC-3 | Y220C | | pancreatic | RPMI-1640 + 10% |
| | | | adenocarcinoma | FBS |
| HCC2935 | Y220C | | lung adenocarcinoma | RPMI-1640 + 10% |
| | | | | FBS |
| MFE-296 | Y220C | R306* | endometrial | EMEM + 10% FBS |
| | (Heterozygous) | (Heterozygous) | adenocarcinoma | |
| COV362 | Y220C | | ovarian epithelial- | DMEM + 10% FBS |
| | | | endometroid carcinoma | |
| KON | Y220C | | oral squamous carcinoma | DMEM + 10% FBS |
| HuH-7 | Y220C | | hepato cellular carcinoma | DMEM + 10% FBS |
| TE-8 | Y220C | M271I | Esophageal carcinoma | RPMI-1640 + 10% |
| | (Heterozygous) | (Heterozygous) | | FBS |
| NUGC-3_KO | KO | | gastric adenocarcinoma | RPMI-1640 + 10% |
| | | | | FBS |
| SJSA-1 | WT | | osteosarcoma | RPMI-1640 + 10% |
| | | | | FBS |
| HCT 116 | WT | | colorectal carcinoma | Mccoy's 5A + 10% |
| | | | | FBS |
| TOV-112D | R175H | | ovarian endometroid | DMEM + 10% FBS |
| | | | carcinoma | |
| NCI-H2029 | Y220D | | small cell lung carcinoma | DMEM: F12-HITES + |
| | | | | 5% FBS |
| SU.86.86 | G245S | G360V | pancreatic carcinoma | RPMI-1640 + 10% |
| | | | | FBS |
| SF-295 | R248Q | | glioblastoma | RPMI-1640 + 10% |
| | | | | FBS |
| C-33 A | R273C | | retinoblastoma | EMEM + 10% FBS |
| A-431 | R273H | | epidermoid carcinoma | DMEM + 10% FBS |
| EFE184 | R282W | | endometrial carcinoma | RPMI-1640 + 10% |
| | | | | FBS |

# homozygous, except indicated; fs, frameshift;
*Stop.

Cell lysate and RNA Preparation: Cells plated and treated in 96-well plates (or in 384-well plates) were quickly washed with 100 μL (or 30 μL) FCW per well using a Blue Washer and GentleSpin evacuation. The cells were then immediately lysed using FastLane Lysis Buffer along with gDNA Wipeout Buffer 2 from a FastLane Cell Probe Kit using 40 μL (or 15 40 μL for 384-well plates) for each well. Cell lysates were heated to 75° C. for 5 minutes before being diluted and immediately measured by RT-qPCR or stored at −80° C. for later analysis. The total RNA was purified from cells in the 6-well plate. Briefly, the medium was aspirated and then cells were immediately lysed in 350 μL Buffer RLT supplemented with 10 μL/mL of β-mercaptoethanol. RNA was purified ether manually by using an RNeasy Mini Kit with DNase I digestion or automatically using QIAcube with DNase digestion. RNA concentration was measured using a NanoDrop 2000 Spectrophotometer.

RT-qPCR: Cell lysates in FastLane lysis buffer were diluted 10-20 times in RNase-free water before using 4 μL of cell lysate in each RT-qPCR assay in 20 μL reactions using a LightCycler 96 or LightCycler 480. In each assay, a QuantiTect Probe RT-PCR Kit was used along with the specific TaqMan primer/probe sets as indicated in individual channels. The expression of a gene of interest (e.g. p21) relative to the reference gene GAPDH in the ratio to the DMSO control was calculated using the ΔΔCt method.

p53 pathway gene profiling: The p53 signaling pathway profiling was conducted by SYBR Green-based real-time qPCR after reverse transcription. The first strand cDNA was synthesized from 500 ng purified total RNA from each sample of biological triplicates (n=3) using an RT² First Strand Kit before being mixed with RT² SYBR Green qPCR Mastermix. Subsequently, the mixture was applied to the RT² Profiler™ PCR Array Human p53 Signaling Pathway—Plate F and detected by LightCycler 96. Data were analyzed using the average Ct values of 5 housekeeping genes on the plate as the reference control to normalize plate-to-plate variation. Alternatively, a similar result was achieved by the ΔΔCt method using 5 housekeeping genes as the first reference control and the DMSO control group as the second reference control.

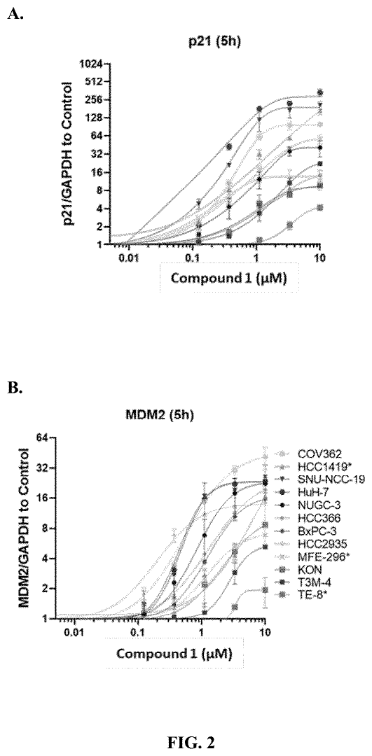

FIG. 2 PANEL A and PANEL B show that Compound 2 activates transcription of p53 target genes p21 and MDM2 in a dose-dependent manner in all Y220C mutant p53 carrying cell lines tested, representing diverse tissue origins. Twelve cell lines harboring p53 Y220C mutation as indicated were treated with compounds Compound 2 for 5 h before p21 and MDM2 mRNA being analyzed by RT-qPCR using LightCycler 480. * Heterozygous Y220C with another p53 allele mutation: MFE-296: Y220C/R306*; HCC-1419: Y220C, A74fs*; TE-8: Y220C, M237I. The data are represented as fold increases compared to DMSO treated cells. The p21 responses ranged from a ˜4-fold increase for TE8 (a heterozygous line carrying Y220C and M271I mutations) to the homozygous line HUH7 reaching a 335-fold increase. MDM2 responses ranged from a 1.9-fold increase for TE8 to the homozygous line COV362 reaching a 41-fold increase. TABLE 11 shows maximum-fold p21 and MDM2 induction levels following Compound 2 treatment across cell lines with Y220C p53 compared to those observed from other lines lacking the Y220C p53 protein target. Cells were treated with up to 10 μM Compound 2 for 5 h before p21 and MDM2 mRNA being analyzed by RT-qPCR. The data support the hypothesis that Compound 2 has activity across a broad array of tumor settings.

TABLE 11

| Cell | p53 | Additional p53 | p21 | MDM2 |
|---|---|---|---|---|
| Line | Status ^{#} | Mutation | Fold | Fold |
| COV362 | Y220C | | 100 | 41 |
| NUGC-3 | Y220C | | 58 | 33 |
| SNU-NCC-19 | Y220C | | 208 | 26 |
| HuH-7 | Y220C | | 335 | 24 |
| HCC366 | Y220C | | 14 | 19 |
| HCC1419 | Y220C (Heterozygous) | A74fs*47 | 167 | 17 |
| | | (Heterozygous) | | |
| BxPC-3 | Y220C | | 9.4 | 16 |
| HCC2935 | Y220C | | 115 | 14 |
| KON | Y220C | | 9.8 | 8.7 |
| MFE-296 | Y220C (Heterozygous) | R306* (Heterozygous) | 57 | 6.8 |
| T3M-4 | Y220C | | 23 | 6.4 |
| TE-8 | Y220C (Heterozygous) | M271I (Heterozygous) | 4.2 | 1.9 |
| TOV-112D | R175H | | 1.0 | 1.1 |
| SU.86.86 | G245S | G360V | 1.2 | 1.1 |
| SJSA-1 | WT | | 1.1 | 1.1 |
| A-431 | R273H | | 1.1 | 1.1 |
| EFE184 | R282W | | 2.0 | 1.1 |
| HCT 116 | WT | | 1.3 | 1.0 |
| NCI-H2029 | Y220D | | 1.2 | 1.0 |
| C-33 A | R273C | | 1.1 | 1.0 |
| SF295 | R248Q | | 1.2 | 1.0 |
| NUGC-3_KO | KO | | 1.0 | 1.0 |

^{#}, Homozygous except indicated.
fs, frameshift;
*Stop.

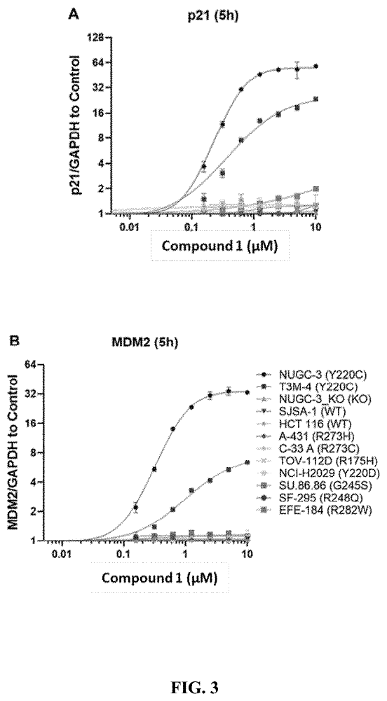

The effects of Compound 2 on cells with different p53 status other than Y220C mutation were investigated. Twelve cell lines with different p53 status as indicated were treated with a dose range of Compound 2 for 5 h at which time p21 and MDM2 mRNA levels were quantified by RT-qPCR using a LightCycler 96. Compound 2 was highly selective for lines harboring the mutant p53 Y220C, with almost no transcriptional activation of the p53 target genes p21 or MDM2 observed in cell lines not carrying the p53 Y220C mutation. Maximal observed increases of p21 following Compound 2 treatment in lines with WT p53, without p53 (KO) or those carrying mutations elsewhere in the p53 gene, ranged from 1.0 (NUGC-3_KO, NUGC-3 with p53 knocked out) to 2-fold (EFE184 harboring p53 R282W). Maximal observed increases of MDM2 ranged from 1.0 (NUGC-3 KO) to 1.1-fold (TOV-112D harboring p53 R175H). FIG. 3 PANEL A and PANEL B show activity and selectivity of Compound 2 in cells harboring Y220C p53 mutation, but not cells without p53 (KO) or cells with either WT or different mutations of p53.

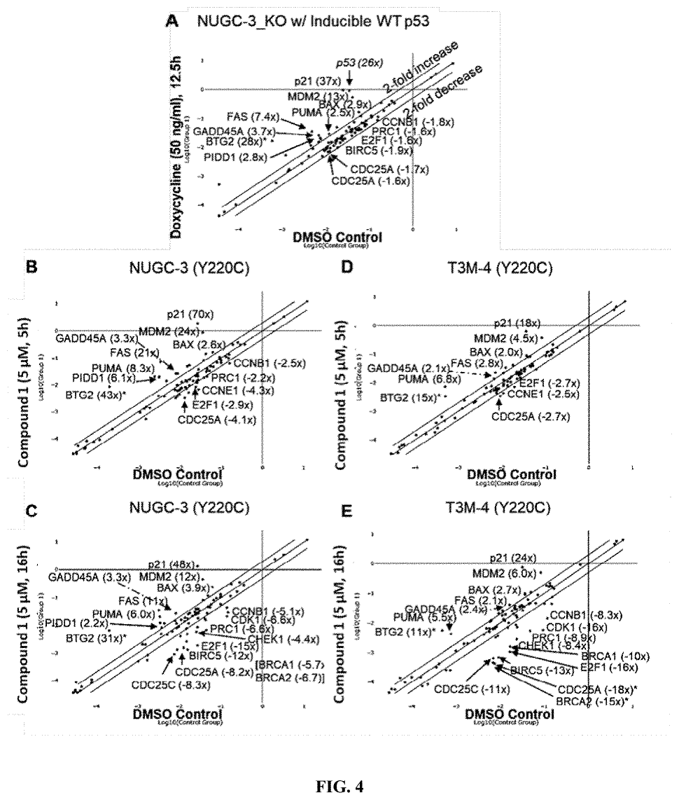

A broader gene expression profile for 84 p53-related pathway genes was investigated. FIG. 4 PANEL A-PANEL E show a visualization of transcriptional changes following treatment with Compound 2. RNA samples from NUGC-3 and T3M-4 cells treated with Compound 2 (5 μM, 5 h and 16 h) and NUGC-3_KO_p53i cells induced with doxycycline (50 ng/ml, 12.5 h) in triplicate were analyzed. p53 pathway gene expression profile patterns in NUCG-3 and T3M-4 cells treated with Compound 2 were similar to that in NUGC-3_KO with inducible WT p53 (NUGC-3 KO_p53/), suggesting that Compound 2 restores Y220C mutant p53 to WT p53 function. A robust response following doxycycline induction of exogenous WT p53 expression required a longer (12.5 h) period compared to Compound 2 treated NUGC-3 cells (5 h).

TABLE 12 shows fold changes of the most changed and profiled p53 pathway genes. The data compare a response in NUGC-3 and T3M-4 cells treated with Compound 2 vs. NUGC-3_KO with inducible WT p53, and NUGC-3_KO and SJSA-1 treated with Compound 2. RNA samples from NUGC-3, T3M-4, NUGC-3_KO, and SJSA-1 cells treated with Compound 2 (5 μM, 5 h and 16 h) and NUGC-3_KO_p53i cells induced with Doxycycline (50 ng/ml, 12.5 h) were analyzed. * indicates the basal expression level is low and thus the fold change may need more biological replicates to validate. Data are the means of fold changes±Standard Deviations (n=3).

TABLE 12

| | NUGC- | NUGC-3 + | | T3M-4 + |
|---|---|---|---|---|
| | 3KO_p53i | Compound | | Compound 2 |
| Gene | 12.5 h | 5 h | 16 h | 5 h |
| TP53 | 25.95 ± 0.84 | 0.86 ± 0.04 | 0.68 ± 0.03 | 0.85 ± 0.03 |
| CDKN1A | 37.37 ± 0.55 | 70.50 ± 2.84 | 48.15 ± 2.12 | 17.67 ± 0.29 |
| BTG2* | 27.82 ± 2.42 | 42.71 ± 3.73 | 31.33 ± 3.24 | 14.58 ± 0.52 |
| MDM2 | 12.97 ± 0.29 | 24.30 ± 1.11 | 12.48 ± 0.78 | 4.51 ± 0.08 |
| FAS | 7.34 ± 0.04 | 21.00 ± 1.28 | 10.91 ± 0.52 | 2.78 ± 0.06 |
| BBC3 | 2.46 ± 0.06 | 8.37 ± 0.62 | 6.04 ± 0.15 | 6.64 ± 0.25 |
| SESN2 | 2.50 ± 0.12 | 6.34 ± 0.12 | 3.58 ± 0.19 | 3.19 ± 0.07 |
| PIDD1 | 2.75 ± 0.05 | 6.10 ± 0.35 | 2.21 ± 0.19 | 1.31 ± 0.18 |
| TNFRSF10B | 1.88 ± 0.04 | 3.35 ± 0.11 | 2.43 ± 0.05 | 2.47 ± 0.15 |
| GADD45A | 3.68 ± 0.20 | 3.30 ± 0.40 | 3.28 ± 0.31 | 2.05 ± 0.09 |
| IL6* | 1.23 ± 0.01 | 3.27 ± 0.14 | 1.55 ± 0.05 | 0.95 ± 0.37 |
| TNFRSF10D | 2.73 ± 0.04 | 3.15 ± 0.13 | 1.54 ± 0.10 | 2.34 ± 0.12 |
| PPM1D | 2.21 ± 0.10 | 2.99 ± 0.04 | 2.68 ± 0.03 | 2.11 ± 0.08 |
| APAF1 | 2.40 ± 0.16 | 2.95 ± 0.16 | 1.35 ± 0.07 | 1.51 ± 0.02 |
| BAX | 2.85 ± 0.08 | 2.59 ± 0.11 | 3.88 ± 0.11 | 2.04 ± 0.04 |
| CCNG 1 | 1.65 ± 0.05 | 2.20 ± 0.06 | 2.18 ± 0.06 | 1.88 ± 0.05 |
| EGR1* | 4.40 ± 0.50 | 0.74 ± 0.03 | 0.50 ± 0.12 | 1.49 ± 0.11 |
| DNMT1 | 0.78 ± 0.03 | 0.70 ± 0.03 | 0.36 ± 0.01 | 0.75 ± 0.02 |
| CHEK2 | 0.74 ± 0.01 | 0.69 ± 0.05 | 0.38 ± 0.04 | 0.79 ± 0.05 |
| CDK1 | 0.71 ± 0.01 | 0.66 ± 0.02 | 0.15 ± 0.00 | 0.87 ± 0.05 |
| BRCA1 | 0.67 ± 0.03 | 0.50 ± 0.02 | 0.17 ± 0.00 | 0.67 ± 0.03 |
| MSH2 | 0.70 ± 0.02 | 0.48 ± 0.05 | 0.42 ± 0.02 | 0.62 ± 0.03 |
| CHEK1 | 0.71 ± 0.02 | 0.46 ± 0.02 | 0.23 ± 0.02 | 0.58 ± 0.01 |
| PRC1 | 0.64 ± 0.01 | 0.45 ± 0.02 | 0.15 ± 0.00 | 0.76 ± 0.01 |
| BRCA2 | 0.74 ± 0.02 | 0.44 ± 0.01 | 0.15 ± 0.01 | 0.60 ± 0.06 |
| CDC25C | 0.58 ± 0.02 | 0.40 ± 0.04 | 0.12 ± 0.01 | 0.80 ± 0.02 |
| CCNB1 | 0.57 ± 0.01 | 0.40 ± 0.01 | 0.20 ± 0.01 | 0.72 ± 0.02 |
| E2F1 | 0.63 ± 0.02 | 0.34 ± 0.01 | 0.07 ± 0.00 | 0.37 ± 0.03 |
| BIRC5 | 0.52 ± 0.03 | 0.32 ± 0.02 | 0.08 ± 0.01 | 0.73 ± 0.06 |
| CDC25A | 0.61 ± 0.04 | 0.25 ± 0.01 | 0.12 ± 0.02 | 0.37 ± 0.05 |
| CCNE 1 | 0.70 ± 0.01 | 0.24 ± 0.01 | 0.31 ± 0.02 | 0.41 ± 0.01 |

| | T3M-4 + | NUGC-3_KO + | | SJSA-1 + | |
|---|---|---|---|---|---|
| | Compound 2 | Compound 2 | | Compound 2 | |
| Gene | 16 h | 5 h | 16 h | 5 h | 16 h |
| TP53 | 0.72 ± 0.04 | 0.79 ± 0.02 | 0.44 ± 0.34 | 1.00 ± 0.03 | 0.95 ± 0.03 |
| CDKN1A | 23.54 ± 1.45 | 1.29 ± 0.13 | 1.34 ± 0.06 | 1.14 ± 0.08 | 1.03 ± 0.07 |
| BTG2* | 11.44 ± 0.81 | 1.04 ± 0.08 | 1.15 ± 0.08 | 1.20 ± 0.03 | 1.04 ± 0.04 |
| MDM2 | 5.98 ± 0.35 | 1.01 ± 0.06 | 0.98 ± 0.04 | 1.01 ± 0.08 | 1.05 ± 0.07 |
| FAS | 2.08 ± 0.09 | 0.98 ± 0.07 | 0.98 ± 0.10 | 1.00 ± 0.05 | 0.93 ± 0.05 |
| BBC3 | 5.50 ± 0.61 | 1.09 ± 0.04 | 1.00 ± 0.03 | 1.12 ± 0.09 | 1.08 ± 0.05 |
| SESN2 | 2.23 ± 0.02 | 1.11 ± 0.11 | 1.06 ± 0.15 | 1.20 ± 0.12 | 1.19 ± 0.09 |
| PIDD1 | 1.89 ± 0.44 | 1.03 ± 0.15 | 0.92 ± 0.17 | 1.04 ± 0.24 | 1.03 ± 0.37 |
| TNFRSF10B | 2.52 ± 0.12 | 1.07 ± 0.02 | 1.05 ± 0.02 | 1.11 ± 0.09 | 1.10 ± 0.07 |
| GADD45A | 2.35 ± 0.24 | 1.00 ± 0.07 | 1.04 ± 0.05 | 1.06 ± 0.05 | 0.99 ± 0.02 |
| IL6* | 0.89 ± 0.18 | 1.47 ± 0.18 | 1.27 ± 0.19 | 1.41 ± 0.49 | 1.15 ± 0.13 |
| TNFRSF10D | 2.51 ± 0.13 | 0.98 ± 0.10 | 1.02 ± 0.04 | 0.99 ± 0.08 | 1.02 ± 0.05 |
| PPM1D | 1.94 ± 0.05 | 1.06 ± 0.02 | 1.00 ± 0.03 | 1.05 ± 0.04 | 1.06 ± 0.06 |
| APAF1 | 0.94 ± 0.05 | 0.95 ± 0.03 | 0.99 ± 0.05 | 1.06 ± 0.15 | 0.95 ± 0.16 |
| BAX | 2.71 ± 0.20 | 0.98 ± 0.07 | 1.03 ± 0.05 | 1.04 ± 0.06 | 1.04 ± 0.07 |
| CCNG 1 | 2.19 ± 0.05 | 1.03 ± 0.04 | 1.07 ± 0.02 | 1.03 ± 0.01 | 1.08 ± 0.07 |
| EGR1* | 0.89 ± 0.27 | 1.15 ± 0.09 | 1.09 ± 0.08 | 0.98 ± 0.27 | 1.11 ± 0.32 |
| DNMT1 | 0.30 ± 0.03 | 0.94 ± 0.04 | 0.95 ± 0.06 | 1.05 ± 0.08 | 0.98 ± 0.07 |
| CHEK2 | 0.41 ± 0.04 | 1.03 ± 0.10 | 0.99 ± 0.06 | 1.14 ± 0.12 | 1.08 ± 0.11 |
| CDK1 | 0.06 ± 0.01 | 0.96 ± 0.06 | 0.93 ± 0.03 | 1.01 ± 0.04 | 1.00 ± 0.04 |
| BRCA1 | 0.10 ± 0.01 | 0.97 ± 0.01 | 0.97 ± 0.03 | 0.99 ± 0.06 | 0.99 ± 0.02 |
| MSH2 | 0.37 ± 0.02 | 1.00 ± 0.03 | 1.01 ± 0.03 | 1.00 ± 0.04 | 0.97 ± 0.05 |
| CHEK1 | 0.12 ± 0.01 | 0.93 ± 0.06 | 0.96 ± 0.06 | 1.00 ± 0.07 | 0.99 ± 0.07 |
| PRC1 | 0.11 ± 0.01 | 0.97 ± 0.03 | 0.95 ± 0.03 | 1.03 ± 0.03 | 0.94 ± 0.05 |
| BRCA2 | 0.07 ± 0.02 | 0.90 ± 0.04 | 0.92 ± 0.01 | 0.90 ± 0.12 | 0.92 ± 0.04 |
| CDC25C | 0.09 ± 0.02 | 0.98 ± 0.06 | 0.96 ± 0.09 | 0.98 ± 0.07 | 0.98 ± 0.14 |
| CCNB1 | 0.12 ± 0.00 | 0.98 ± 0.05 | 0.96 ± 0.05 | 1.00 ± 0.04 | 1.00 ± 0.10 |
| E2F1 | 0.06 ± 0.01 | 0.94 ± 0.07 | 0.95 ± 0.08 | 1.06 ± 0.02 | 0.98 ± 0.04 |
| BIRC5 | 0.08 ± 0.02 | 0.93 ± 0.08 | 0.94 ± 0.10 | 1.03 ± 0.06 | 0.96 ± 0.06 |
| CDC25A | 0.06 ± 0.01 | 0.96 ± 0.04 | 0.91 ± 0.03 | 1.09 ± 0.05 | 0.99 ± 0.08 |
| CCNE 1 | 0.41 ± 0.04 | 0.94 ± 0.04 | 0.95 ± 0.03 | 0.94 ± 0.01 | 0.91 ± 0.03 |

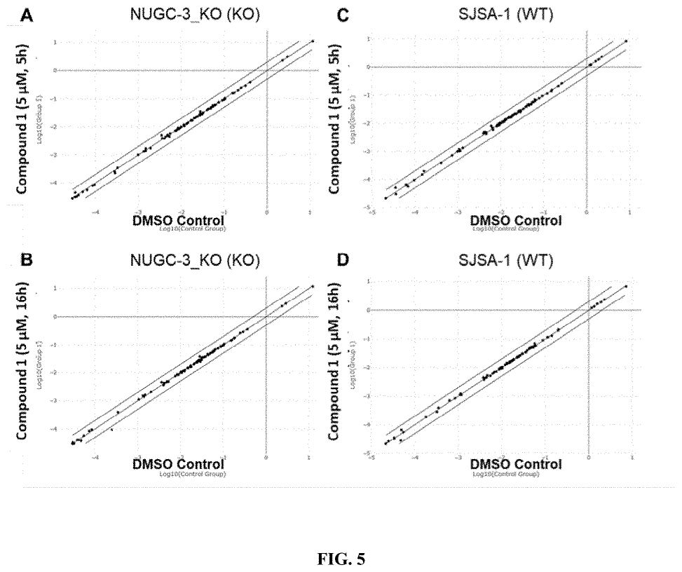

FIG. 5 PANEL A-PANEL D show selectivity of Compound 2 by the 84 p53-related gene panel. Compound 2 exposure resulted in no significant changes in p53 pathway gene expression patterns in either p53 WT or p53 KO cells. The result suggests that Compound 2 activity is specific to restoration of the Y220C mutant p53 protein. No Compound 2-dependent changes in transcription of 84 p53-related genes were observed when Y220C p53 was not present. The p53 regulated gene expression patterns show that Compound 2 effectively restored WT function to Y220C mutant p53 in cells harboring a p53 Y220C mutation. The reactivation of p53 Y220C by Compound 2 was selective as no effect was observed in p53 WT cells, KO cells, or cells with other p53 mutations.

### Example 4: Restoration of p53 Y220C Form and Function by Compound 2

The conformation shift caused by Compound 2 was characterized across several p53-Y220C cell lines, all of which had varying amounts of mutant p53. All cell lines harboring the Y220C p53 mutation were sensitive to treatment with Compound 2. A time course with Compound 2 revealed 2 distinct profiles, one which had a sharp peak of WT p53 and MDM2 protein and the other which had a slow increase in WT p53 and MDM2 protein. NUGC3 has a large pool of mutant p53 and showed regression following Compound 2 in xenograft studies. T3M4 has a smaller pool of mutant p53 and showed tumor growth inhibition following Compound 2 treatment in xenograft studies.

Cell lysate preparation: All cell lines were maintained in proper media and grown in incubators at 37° C. with 5% CO₂. To harvest cells for analysis, media was aspirated, and the cells washed with PBS. Lysis buffer was added to plates, and the cell lysate was harvested using a cell scraper. Homogenized samples were spun by centrifuge for 15 minutes at max speed (14K rpm), and the supernatant was transferred to a clean Eppendorf tube. Protein samples were quantified using BCA. Protein samples were aliquoted and stored at −80° C.

Western blot: For each cell line, 10 μg of total protein were run on a 4-12% Bis-Tris precast gel. The gel was transferred to a nitrocellulose membrane. The membrane was blocked for 1 hour using the Odyssey TBS blocking solution. Membranes were incubated overnight in p53 (p53, DO-1) and actin (beta-actin) primary antibodies. The next day, blots were washed and incubated in either goat anti-mouse for p53 or goat-anti-rabbit for actin secondaries antibodies. Blots were then washed and imaged using an Odyssey CLx imaging system. Bands were quantified using Image Studio software and normalized to the actin control. The p53 or actin levels were then normalized to the NUGC3 band and graphed using Prism.

P53 and MDM2 ELISA: For the p53 conformation ELISA on day 1, ELISA plates were coated with either WT p53 (150 ng/well), Mutant p53 (100 ng/well), or Total p53 (31.3 ng/well) the night before the assay and stored at 4° C. On day 2, plates were washed 3 times with PBS+0.05% Tween-20 (wash buffer) and blocked in PBS+1% BSA+0.05% Tween-20 (blocking buffer) for a minimum of 1 hour, after which the plates were washed 3 times. Cell lysates were diluted in blocking buffer to the appropriate concentration for each ELISA, and 100 μL/well was added to the ELISA plates. Protein concentrations for NUGC3 cell lysates were: WT, 5 μg; mutant, 2.5 μg; total, 1.25 μg. Protein concentrations for T3M4 cell lysates were: WT and mutant, 5 μg; total, 2.5 μg. Plates were incubated overnight at 4° C. with shaking. On day 3, plates were washed 3 times with PBS+0.05% Tween-20 and incubated in detection antibody diluted in blocking buffer (0.025 mg/mL; biotinylated p53) for 1 hour. Plates were washed 3 times and incubated in streptavidin-HRP (1:10000) diluted in blocking buffer for 30 minutes. Plates were washed and developed with TMB for approximately 5 minutes and the reaction quenched with 0.16 M sulfuric acid. Plates were read on a plate reader at 450 nm. The signal from treated samples was normalized to their respective vehicle control.

For the MDM2 ELISA, polystyrene 96 well plates were coated with a capture antibody and incubated overnight at 4° C. Plates were then washed in wash buffer and blocked for 1 h. Cell lysates (10 μg) were diluted to the appropriate concentration and added to a volume of 100 μL. Additionally, a 7-point standard curve was also added to the plates. Plates were incubated at 4° C. overnight with shaking. The plates were then washed and incubated in detection antibody for 2 hours. Plates were washed and incubated in streptavidin-HRP for 30 min. Finally, plates were washed, and the reaction was developed using a TMB substrate for 10 min. The reaction was quenched with a stop solution (0.16 M H₂SO₄), and the plates were read at 450 and 570 nm. Protein levels for both analytes were quantified using the provided standard curve.

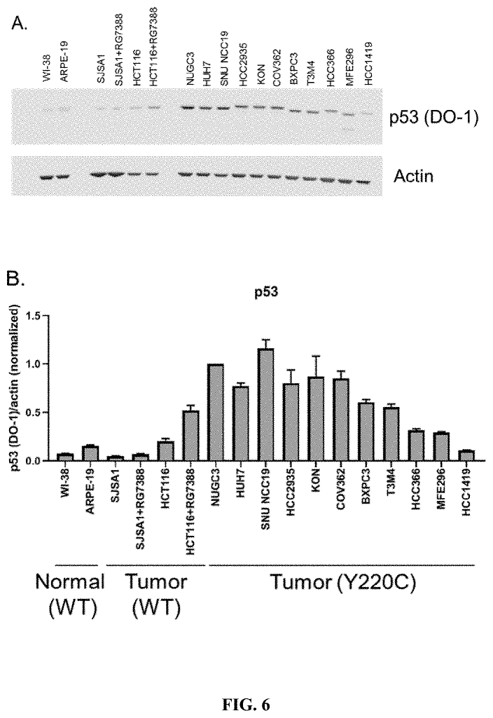

p53 Y220C levels in cells: The levels of p53 are highly regulated in normal tissues, with most cells demonstrating extremely low abundance except following cellular insult such as DNA damage. Since p53 protein pools are strongly autoregulated by degradation by the transcriptional target MDM2, pools of p53 accumulate to high levels in cells with mutant p53. FIG. 6 PANEL A and PANEL B demonstrate the elevated levels within a panel of tumor cell lines harboring p53 Y220C compared to levels found within normal and tumor lines containing WT p53, both untreated and treated with 50 nM RG7388 for 24 hours. The values from quantification of the western blot are shown in TABLE 13.

TABLE 13
Band Density measurement from Image Studio Lite

| | | | | normalized |
|---|---|---|---|---|
| | DO1 | Actin | DO1/Actin | DO1/Actin |
| HCC1419 | 442 | 31700 | 0.014 | 0.112 |
| MFE296 | 1420 | 38900 | 0.037 | 0.293 |
| HCC366 | 1545 | 39500 | 0.039 | 0.315 |
| T3M4 | 1960 | 28100 | 0.070 | 0.561 |
| BXPC3 | 2125 | 28100 | 0.076 | 0.608 |
| COV362 | 2930 | 27600 | 0.106 | 0.855 |
| KON | 2810 | 26100 | 0.108 | 0.873 |
| HCC2935 | 3140 | 31600 | 0.099 | 0.803 |
| SNUNCC19 | 3375 | 23400 | 0.144 | 1.161 |
| HUH7 | 2895 | 30100 | 0.096 | 0.773 |
| NUGC3 | 4200 | 33700 | 0.125 | 1.000 |
| HCT116 + RG7388 | 1300 | 20000 | 0.065 | 0.524 |
| (50 nM, 24 hr) | | | | |
| HCT116 | 560 | 21900 | 0.026 | 0.206 |
| SJSA1 + RG7388 | 94.5 | 46600 | 0.008 | 0.068 |
| (50 nM, 24 hr) 3 | | | | |
| SJSA1 | 303 | 45800 | 0.007 | 0.053 |
| ARPE-19 | 622.5 | 31600 | 0.020 | 0.158 |
| WI-38 | 351.5 | 37700 | 0.009 | 0.075 |

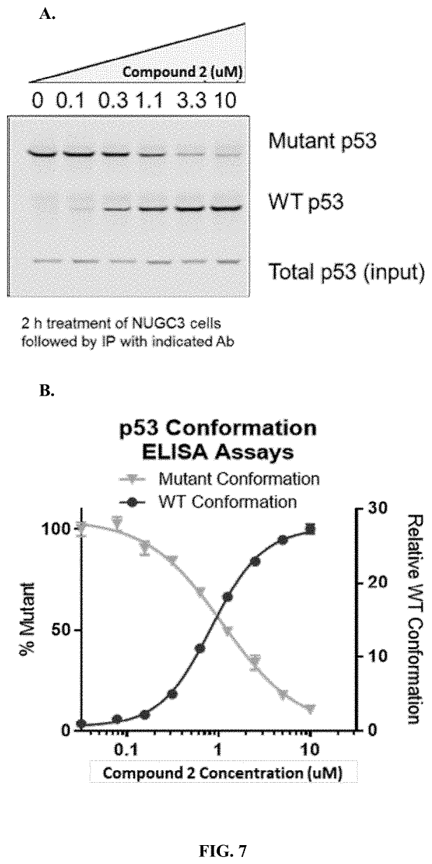

The shift from mutant conformation to wild type conformation when cells are exposed to Compound 2 was evaluated. FIG. 7 PANEL A depicts a p53 Western blot following an immunoprecipitation using mutant specific or wild type (WT) specific antibodies from lysates treated with varying concentrations of Compound 2 for 2 hours. A Compound 2 dose-dependent depletion of mutant conformation concurrent with increased WT conformation was observed, though total levels of p53 within the lysate remains unchanged. FIG. 7 PANEL B shows analysis of the same lysate using ELISAs developed to quantitate mutant or wild type conformation.

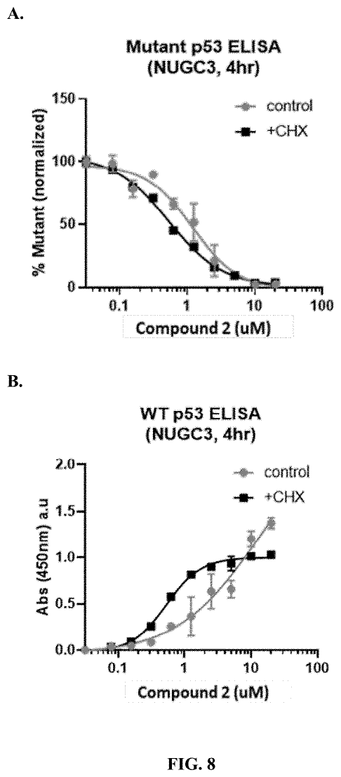

Based on the rapid conversion from mutant p53 to WT conformation p53, further studies were conducted to determine whether the existing pool of mutant p53 could be converted from mutant to WT. Cycloheximide was added to the cell culture media 1 h prior to a 4 h treatment with Compound 2 in NUGC3 cells. The cell lysates were then analyzed using the p53 conformation ELISAs. The addition of cycloheximide did not significantly alter the ability of Compound 2 to convert mutant p53 to WT conformation p53 demonstrating that Compound 2 can shift the conformation of the existing pool of p53 Y220C protein. FIG. 8 PANEL A and PANEL B show that addition of cycloheximide did not affect the ability of Compound 2 to induce mutant to wild type conformation change in NUGC3 cells. Quantification of mutant and WT ELISAs of FIG. 8 are shown in TABLE 14.

TABLE 14

| | | Raw Mutant Values | | Normalized Mutant Values | |
|---|---|---|---|---|---|
| | | (O.D) | | (% DMSO) | |
| | | control | +CHX | control | +CHX |
| 20 | uM | 0.020 | 0.019 | 2.976 | 3.779 |
| 10 | uM | 0.018 | 0.017 | 2.738 | 3.292 |
| 5 | uM | 0.278 | 0.047 | 41.368 | 9.377 |
| 2.5 | uM | 0.142 | 0.079 | 21.060 | 15.781 |
| 1.25 | uM | 0.346 | 0.161 | 51.514 | 32.028 |
| 0.625 | uM | 0.445 | 0.227 | 66.206 | 45.074 |
| 0.3125 | uM | 0.652 | 0.356 | 97.017 | 70.886 |
| 0.15625 | uM | 0.527 | 0.401 | 78.472 | 79.825 |
| 0.078125 | uM | 0.661 | 0.470 | 98.386 | 93.557 |

| | | | | |
|---|---|---|---|---|
| DMSO | 0.672 | 0.503 | 100.000 | 100.000 |

Raw WT Values
(O.D)

| | | control | +CHX |
|---|---|---|---|
| 20 | uM | 1.450 | 1.075 |
| 10 | uM | 1.278 | 1.058 |

| | | |
|---|---|---|
| uM | 0.733 | 0.979 |

| | | | |
|---|---|---|---|
| 2.5 | uM | 0.709 | 0.942 |
| 1.25 | uM | 0.439 | 0.860 |
| 0.625 | uM | 0.333 | 0.621 |
| 0.3125 | uM | 0.161 | 0.302 |
| 0.15625 | uM | 0.127 | 0.137 |
| 0.078125 | uM | 0.118 | 0.080 |

| | | |
|---|---|---|
| DMSO | 0.076 | 0.033 |

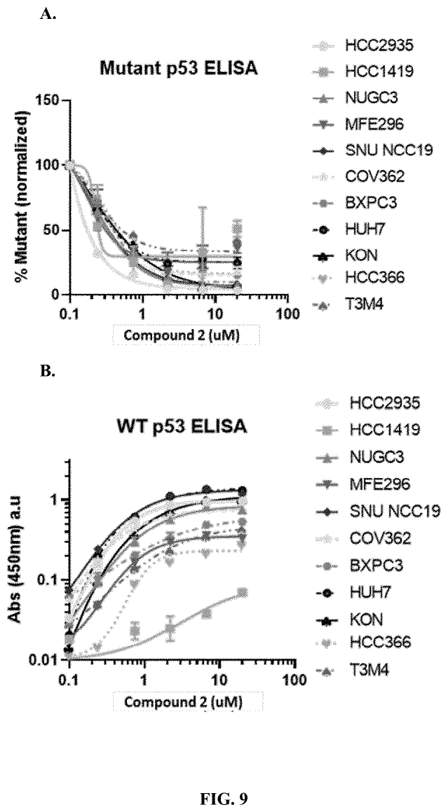

To determine whether Compound 2 acts across tissue and tumor types, 11 cell lines containing the Y220C mutation were treated with a dose range of compound and assessed for gain of WT and loss of mutant conformation at 4 hours. FIG. 9 PANEL A and PANEL B show the conversion from mutant p53 to WT conformation p53 after a 4-hour treatment with Compound 2 in 11 Y220C mutant p53 cell lines. All lines showed robust loss of mutant conformation with a similar dose responsiveness. TABLE 15 shows the EC₅₀ values and quantification of p53 ELISA values of FIG. 9 PANEL A and PANEL B, respectively.

TABLE 15

| Cell line | Mutant p53 EC₅₀ (μM) |
|---|---|
| HCC2935 | 0.063 |
| HCC1419 | 0.258 |
| NUGC3 | 0.348 |
| MFE296 | 0.349 |
| SNU NCC19 | 0.364 |
| COV362 | 0.392 |
| BXPC3 | 0.422 |
| HUH7 | 0.485 |
| KON | 0.52 |
| HCC366 | 0.542 |
| T3M4 | 0.662 |

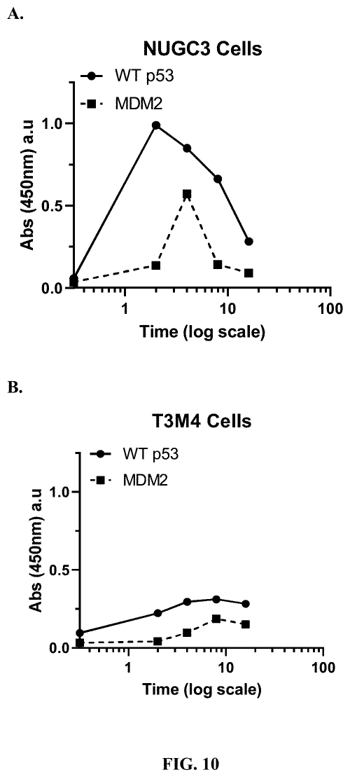

A time course was also performed with 10 μM Compound 2 and timepoints taken between 1 and 12 hours. Two distinct patterns of WT conformation p53 following Compound 2 treatment were observed. FIG. 10 PANEL A and PANEL B show that NUGC3 cells demonstrated rapid onset peaking around 1-2 h after treatment with Compound 2, followed by a sharp drop corresponding with high levels of MDM2; T3M4 exhibited a slow rise in WT conformation to a modest peak around 10 h followed by a decline concurrent with the appearance of modest MDM2 levels. NUGC3 is a very sensitive cell line in cell-based assays, whereas T3M4 is among the least sensitive cell lines.

The basal levels of mutant p53 Y220C were significantly higher than the basal levels of WT p53 in both normal cell lines and WT tumor lines. Using the NUGC3 cell line as a model, the loss of mutant p53 and gain of WT p53 conformation upon addition of Compound 2 was examined. By 2 hours, an increase in WT conformation p53 signal and decrease in mutant p53 signal in a dose dependent manner was observed by both immunoprecipitation and ELISA. The addition of cycloheximide prior to treatment with Compound 2 did not prevent the conversion from mutant to WT conformation p53. The results indicate that the existing pool of mutant p53 protein can be converted to a functional WT protein without the need for newly synthesized p53 protein.

### Example 5: PD/PK Relationship of Compound 1 in a Mouse Syngeneic Model of Sarcoma

The pharmacodynamic and pharmacokinetic (PD/PK) relationship of Compound 1 was measured in a mouse syngeneic model of sarcoma (MT373). C57Bl/6 mice were implanted with MT373 cells, and tumors were grown to a range of ˜200-400 mm³ prior to being randomized into one of three study groups. Mice were dosed orally (PO) with either vehicle control (0.2% HPC, 0.5% Tween 80) or Compound 1 at 75 mg/kg or 150 mg/kg twice per day separated by 8 h (BID×1). Mice (n=4/timepoint) were euthanized and plasma and tumor samples were harvested 8, 24, 48, 72, 96, 144 hours (h) post the first dose for PK and PD analysis.

Study design: The PD response of the test article Compound 1 was evaluated at two dose levels in the syngeneic mouse xenograft model of sarcoma (MT373). Group 1 mice were dosed PO with vehicle control (0.2% HPC, 0.5% Tween 80) BID×1 (7 h). TABLE 16 shows efficacy study groups and dosing regimen. Groups 2, and 3 mice were dosed PO with Compound 1 BID×1 (7 h) at 75 mg/kg or 150 mg/kg. Mice in Groups 1, 2, and 3 (n=4/timepoint) had tumors and plasma harvested for PD/PK analysis at 8, 24, 48, 72, 96, and 144 h post-dose.

TABLE 16

| | | | | Dosing | | |
|---|---|---|---|---|---|---|
| | | | | Frequency & | Dose | Harvest timepoints |
| Group | Treatment | N | Route | Duration | (mg/kg) | (h post last dose) |
| 1 | Vehicle Control | 24 (4/tp) | PO | BIDx1 | N/A | 8, 24, 48, 72, 96, 144 |
| 2 | Compound 1 | 24 (4/tp) | PO | BIDx1 | 75 | 8, 24, 48, 72, 96, 144 |
| 3 | Compound 1 | 24 (4/tp) | PO | BIDx1 | 150 | 8, 24, 48, 72, 96, 144 |

Animals: C57Bl/6 mice (100 total) were purchased from Charles River Labs. Animals were acclimatized for 4 weeks and were 8-10 weeks old at initiation of study. Animals were group housed (N=5) in ventilated cages. Fluorescent lighting was provided on a 12-hour cycle (6:30 am-6:30 pm). Temperature and humidity were monitored and recorded daily and maintained between 68-72° F. (20-22.2° C.) and 30-70% humidity, respectively. 18% soy irradiated rodent feed and autoclaved acidified water (pH 2.5-3) was provided ad libitum.

Tumor Cell Culture: MT373 cells were cultured in DMEM media with 10% fetal bovine serum. The cells were washed with PBS and counted at a total of 8.3×10⁸ cells with 96.5% viability. Cells were spun by centrifuge and resuspended in 50% PBS:50% Matrigel Matrix at a concentration of 5×10⁶ viable cells/200 μL.

Implantation of Mice: Cells were prepared for injections by drawing the cell suspension into a 1 mL tuberculin syringe fitted with a 25G ⅝″ needle. Individual mice were manually restrained, the site of injection (right flank) was disinfected with a 70% ethanol swab, and 200 μL of cell suspension was injected subcutaneously.

Randomization and Study Setup: Implanted animals were monitored for palpable tumors. Eighteen days post implant the animals with palpable tumors had their tumor sizes determined via digital caliper. Mice were selected and randomized into three treatment groups according to tumor size. Average tumor volume (mm³) and body weight (g) are described in TABLE 17. Treatment began on the eighteenth day post-implant to facilitate twice daily dosing.

TABLE 17

| | | Tumor Volume (mm³) | | | | Body Weight (g) | | | |
|---|---|---|---|---|---|---|---|---|---|
| Group | N | Mean | St Dev | Min | Max | Mean | St Dev | Min | Max |
| 1 - Vehicle Control | 24 | 280.75 | ±96.16 | 195.27 | 458.50 | 19.68 | ±1.65 | 17.0 | 22.7 |
| 2 - Compound 1 at | 24 | 309.27 | ±61.77 | 231.23 | 423.20 | 20.47 | ±1.62 | 17.0 | 23.8 |
| 75 mg/kg BIDx1 | | | | | | | | | |
| 3 - Compound 1 at | 24 | 320.34 | ±70.63 | 252.49 | 421.51 | 20.14 | ±1.57 | 16.8 | 22.8 |
| 150 mg/kg BIDx1 | | | | | | | | | |

Measurements and Calculation of Tumor Volume: Tumor volume was calculated using the following equation: (longest diameter×shortest diameter²)/2. Individual tumor volumes and body weight measurements were taken at the final timepoint. The calculation for percent tumor growth inhibition (TGI) is as follows: [1−((Tₜ−T₀/Cₜ−C₀))]×100, where Cₜ is the mean tumor volume of the vehicle control group at time t, C₀ is the mean tumor volume of the vehicle control group at time 0, and T is the mean tumor volume of the treatment group. Tumor regression was determined with the equation [(T₀−Tₜ)/T₀]×100 using the same definitions.

Tumor Lysate Preparation: Lysis buffer was added to tumor samples and homogenized using a TissueLyser LT. Homogenized samples were spun by centrifuge for 30 minutes at 20817×g, and the supernatant transferred to a 1.5-mL tube. Protein samples were quantified, aliquoted into 96-well plates, and stored at −80° C.

Purification of RNA: Frozen tumor samples with the required weight were lysed in Buffer RLT with 10 μL/mL of β-mercaptoethanol using a TissueLyser LT. Total RNA was further purified from the lysate using QIAcube with DNase digestion, and RNA concentration was measured with a NanoDrop 2000 Spectrophotometer.

Mutant, WT, and total p53 ELISA: 96-well ELISA plates were coated with either WT p53 (150 ng/well), mutant p53 (250 ng/well), or total p53 (62.5 ng/well) antibodies and incubated overnight at 4° C. Plates were washed with was buffer (PBS+0.05% Tween 20) and treated with blocking buffer (PBS+1% BSA+0.05% Tween 20) for 1 h and then washed. Tumor lysates were diluted in blocking buffer such that the required protein amount is added to the plate in a 100 μL volume (WT p53 7.5 μg; mutant p53 2.5 μg; Total p53 2.5 μg). Lysates were incubated overnight at 4° C. with shaking. Plates were again washed and treated with detection antibody diluted in blocking buffer (0.625 μg/mL for mutant p53; 0.156 μg/ml for total p53 and 0.3 μg/ml for WT p53) for 1 h, washed, then incubated in Rabbit-HRP (1:100) diluted in blocking buffer for 1 h. Plates were washed, and the reaction was developed using TMB for approximately 5 minutes and quenched with 0.16 M sulfuric acid. Plates were read on a plate reader at 450 nm. A background measurement was subtracted from the treated samples' signals. The signals were normalized to the respective vehicle controls.

p21, MDM2, GDF15, and GAPDH Gene Expression: Individual gene expression was analyzed by one-step TaqMan-based real-time RT-qPCR. Purified total RNA was diluted to 2.5 ng/μL in DNase- and RNase-free water, and 10 ng was used for each 20 μL RT-qPCR assay using a LightCycler 96. A QuantiTect Probe RT-PCR Kit was used along with a specific primer/probe set as indicated. LAM-MGB labels were used for genes GAPDH, CDKN1a, MDM2, GDL15, and TRP53. The expression of the reference gene (human GAPDH and/or mouse GAPDH as indicated) in the ratio to the vehicle control was calculated using the ΔCₜ method with normalization to the total RNA input. The expression of a gene of interest relative to the reference gene was calculated using the ΔCₜ method, and the expression of a gene of interest relative to the reference gene in the ratio to the vehicle control was calculated using the ΔΔCₜ method.

p53 Signaling pathway and NF-κB signaling pathway profiling panels: The signaling pathways were profiled by SYBR Green-based real-time qPCR after reverse transcription. The first strand cDNA was synthesized from 500 ng purified total RNA of each tumor sample with an RT2 First Strand Kit before being mixed with RT2 SYBR Green qPCR Mastermix. Subsequently, the mixture was applied to a RT² Profiler™ PCR Array plate as indicated. RT² Profiler™ PCR Array Mouse p53 Signaling Pathway—Plate F and/or RT² Profiler™ PCR Array Mouse NFkB Signaling Pathway—Plate F and detected by a LightCycler 96. At least 3 samples in each group were used for the profiling. Data were analyzed using Cₜ values of profiled genes resulted from all groups of samples and the average Cₜ values of 5 housekeeping genes on the plate as the reference control to normalize plate-to-plate variation. Alternatively, a similar result was achieved with the ΔΔCₜ method using 5 housekeeping genes as the first reference control and the vehicle group as the second reference control. The cutoff of fold change=2 and p-value=0.05 was applied to curate the data and to eliminate some low expression genes (Cₜ<30).

Mutant, WT and total p53 ELISA PD/PK Results: Mice in Groups 1, 2, and 3 were harvested for PD/PK analysis at 8, 24, 48, 72, 96, and 144 h post first dose. Tumors from mice treated with the highest dose of 150 mg/kg Compound 1 showed a 82.30% decrease in mutant levels of p53 8 h post-dose. The level further reduced to 93.74% by 24 h post-dosing. Plasma concentrations were highest between 11435 ng/mL and 5986 ng/mL. As plasma concentrations started to decrease, the levels of p53 began to rise. At 48 and 72 h post-dose, a 81.64% and 58.64% decrease in mutant level was observed, respectively, and levels of mutant p53 returned to vehicle control levels by 96 h post-dose. In these same tumors, levels of WT conformation p53 increased by 1.45-fold 8 h post dose and returned to baseline by 24 h.

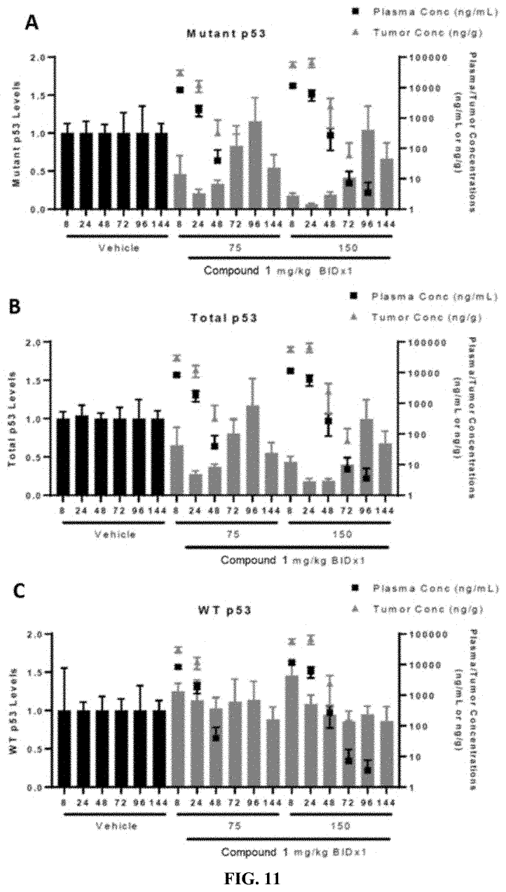

At the lower dose of 75 mg/kg BID×1, mutant levels of p53 decreased by 53.91% and 79.50% at 8 and 24 h, respectively. The observed decrease correlated with plasma concentrations of Compound 1 being at 8375 ng/mL and 1843 ng/mL at these timepoints. After the 24 h timepoint, levels of mutant p53 increased as plasma concentrations decreased, returning to control levels by 72 h. Analysis of these samples for WT conformation p53 showed a 1.25-fold induction of WT conformation p53 8 h post dose, which returned to baseline by 24 h post-dose. FIG. 11 PANEL A-PANEL C show changes in mutant p53, total p53, and WTp53 plasma concentration (ng/mL) and tumor concentration (ng/g) overtime for mice treated with vehicle, 75 mg/kg of Compound 1 BID×1, and 150 mg/kg of Compound 1 BID×1. Levels of total p53 mirrored changes seen in mutant p53 at both dose levels. Compound 1 was well tolerated during the PD study with no effect on tumor control in the 4 day study.

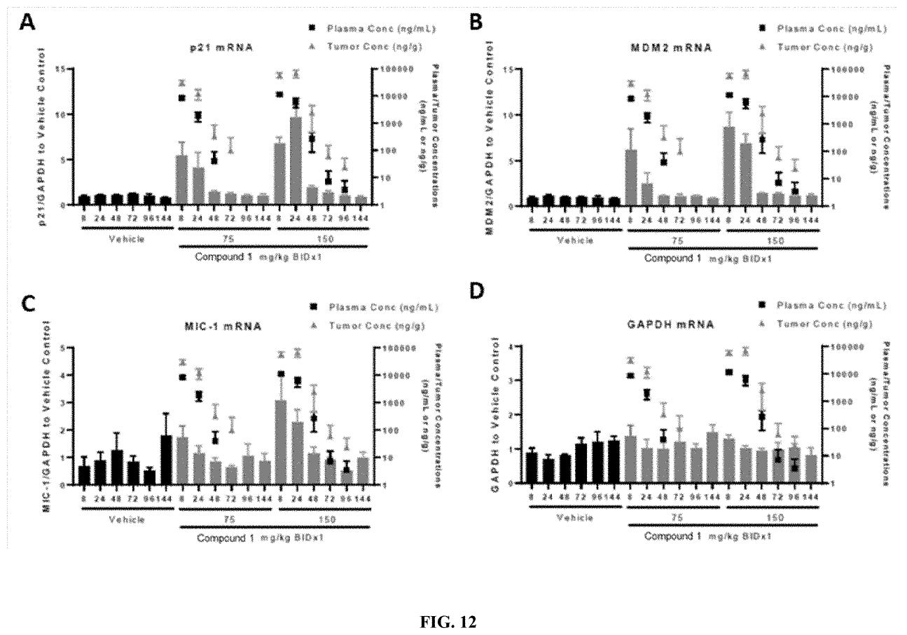

Measurement of target gene mRNAs downstream of WT p53 resulted in a 5.4-fold and 4.0-fold increase in p21 and a 6.2-fold and 2.4-fold increase in MDM2 in the tumors of mice treated with 75 mg/kg BID×1 at 8 and 24 h, respectively. Following the 24 h timepoint levels, the mRNA levels returned to baseline. Tumors from mice dosed with 150 mg/kg BID×1 showed a 6.7 and 9.6-fold increase in p21 mRNA and 8.7 and 6.9-fold increase in MDM2, 8 and 24 h post-dose, respectively. MIC-1 levels increased in the 150 mg/kg BID×1 groups at 8 and 24 h post-dose with a 3.07-fold and 2.29-fold increase, respectively. GAPDH mRNA levels remained consistent between the groups and were used to normalize the gene expression data. FIG. 12 PANEL A-PANEL D shows changes in target gene mRNAs downstream of WTp53 upon treatment with vehicle, 75 mg/kg Compound 1 BID×1, and 150 mg/kg Compound 1 BID×1 over time. TABLE 18 shows the fold change of mutant p53 protein, WT conformation p53 protein, total p53 protein, p21 mRNA, MDM2 mRNA, and MIC-1 mRNA over vehicle control or percent reduction relative to vehicle control in %.

TABLE 18

| | | | | Fold Change over Vehicle Control or Percent Reduction | | | | | |
|---|---|---|---|---|---|---|---|---|---|
| Group - | | | | Relative to Vehicle Control (%) | | | | | |
| Comp 1 | | Plasma | Tumor | Mutant | WT | Total | | | |
| (mg/kg) | Timepoint | Conc. | Conc. | p53 | p53 | p53 | p21 | MDM2 | MIC-1 |
| BIDx1 | (h) | (ng/mL) | (ng/g) | Protein | Protein | Protein | mRNA | mRNA | mRNA |
| Group 2 - | 8 | 8375 | 30853 | 53.91% | 1.25 | 35.06% | 5.43 | 6.16 | 1.74 |
| 75 | 24 | 1843 | 12035 | 79.50% | 1.13 | 72.38% | 4.04 | 2.44 | 1.14 |
| | 48 | 40 | 318 | 67.23% | 1.02 | 63.03% | 1.43 | 1.12 | 0.85 |
| | 72 | 0 | 97 | 16.87% | 1.11 | 19.65% | 1.18 | 1.08 | 0.65 |
| | 98 | 0 | 0 | 0.00% | 1.13 | 0.00% | 0.99 | 1.11 | 1.05 |
| | 144 | 0 | 0 | 45.58% | 0.88 | 44.67% | 0.98 | 0.85 | 0.88 |
| Group 3 - | 8 | 11435 | 57645 | 82.30% | 1.45 | 56.64% | 6.73 | 8.70 | 3.07 |
| 150 | 24 | 5986 | 67778 | 93.74% | 1.08 | 81.75% | 9.61 | 6.88 | 2.29 |
| | 48 | 268 | 2436 | 81.64% | 0.94 | 81.46% | 1.87 | 1.43 | 1.14 |
| | 72 | 7 | 59 | 58.64% | 0.86 | 60.43% | 1.33 | 1.33 | 0.93 |
| | 98 | 3 | 23 | 0.00% | 0.95 | 0.60% | 1.01 | 1.12 | 0.53 |
| | 144 | 0 | 0 | 34.14% | 0.86 | 32.40% | 0.85 | 1.16 | 1.00 |

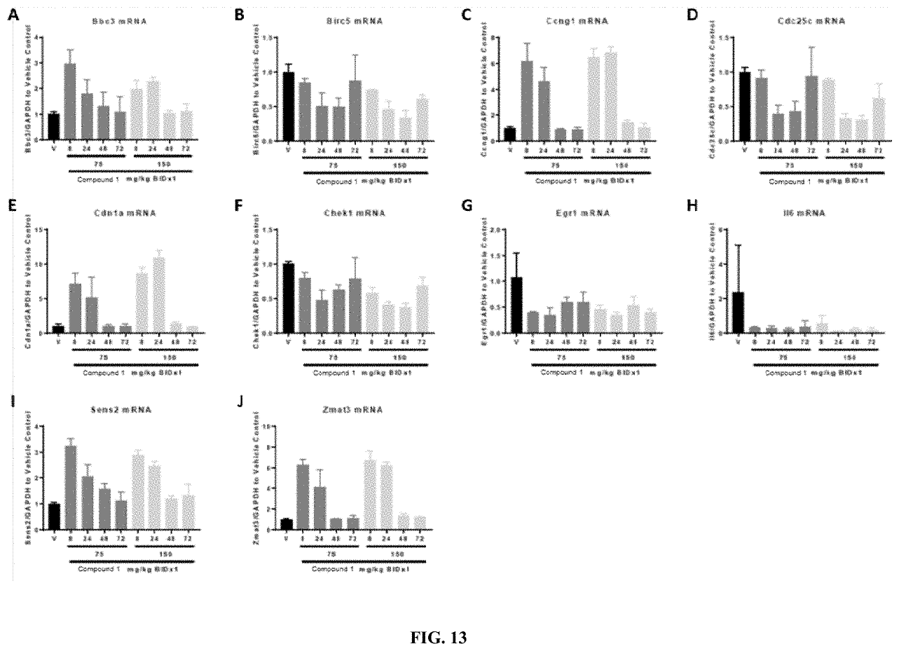

p53 Signaling Pathway and NF-κB Signaling Pathway Profiling PD Results: Measurement of p53 target protein transcripts downstream of WT p53 using a mouse p53 gene expression profiling panel showed changes in genes related to apoptosis in tumors treated with Compound 1 at 75 mg/kg (i.e., Bbc3 1.80-fold increase and Birc5 45.92% decrease at 24 h) and 150 mg/kg (i.e., Bbc3 2.27-fold increase and Birc5 53.57% decrease at 24 h) BID×1 at several timepoints. FIG. 13 show changes in Bbc3 (PANEL A), Birc5 (PANEL B), Ccng1 (PANEL C), Cdc25c (PANEL D), Cdn1a (PANEL E), Chek1 (PANEL F), Egr1 (PANEL G), 116 (PANEL H), and Zmat3 (PANEL I) mRNA levels upon treatment with vehicle, 75 mg/kg Compound 1 BID×1, and 150 mg/kg Compound 1 BID×1 over time. TABLE 19 shows fold increase of genes over vehicle control or percent reduction compared to vehicle control in % upon treatment with vehicle, 75 mg/kg Compound 1 BID×1, and 150 mg/kg Compound 1 BID×1 over time.

Fold increase and percent decrease changes were observed in genes related to cell cycle control in tumors treated with Compound 1 at 75 mg/kg BID×1 (i.e., Ccng1 4.63-fold, Cdc25c 60.54%, Cdk1 59.92%, Cdkn1a 5.16-fold, Chek1 52.36%, Zmat3 4.14-fold at 24 h) and 150 mg/kg BID×1 (i.e., Ccng1 6.84-fold, Cdc25c 41.75%, Cdk1 64.58%, Cdkn1a 10.93-fold, Chek1 58.77%, Zmat3 6.17-fold at 24 h) at several timepoints. Other genes were significantly upregulated or downregulated in tumors treated with Compound 1 at 75 and 150 mg/kg BID×1 related to growth and proliferation (i.e., Egfr 28.18% and 47.37% at 24 h, respectively), inflammation and immune response (Il6 70.78% and 85.31% at 24 h, respectively), ubiquitination (Mdm2 2.76-fold and 5.96-fold at 24 h, respectively) and cell growth (Sesn2 2.05-fold and 2.45-fold at 24 h, respectively). Six control housekeeping genes were included in the panel.

TABLE 19
Fold Increase Over Vehicle Control or Percent Reduction
Compared to Vehicle Control (%) ± St. Dev.

| | | | Compound 1 at 75 mg/kg | | | Compound 1 at 150 mg/kg | | |
|---|---|---|---|---|---|---|---|---|
| | | | BIDx1 | | | BIDx1 | | |
| Gene | 8 h | 24 h | 48 h | 72 h | 8 h | 24 h | 48 h | 72 h |
| Apaf1 | 1.30 ± | 1.03 ± | 15.51 ± | 19.88 ± | 1.28 ± | 1.10 ± | 19.34 ± | 10.88 ± |
| | 0.11 | 0.12 | 3.53 | 15.81 | 0.11 | 0.10 | 10.20 | 15.94 |
| Apex1 | 19.97 ± | 30.49 ± | 21.30 ± | 24.49 ± | 27.35 ± | 29.90 ± | 31.42 ± | 15.68 ± |
| | 7.95 | 9.54 | 4.68 | 16.70 | 7.35 | 3.84 | 2.41 | 13.49 |
| Atm | 13.42 ± | 23.68 ± | 18.31 ± | 12.27 ± | 22.22 ± | 24.13 ± | 26.33 ± | 18.10 ± |
| | 11.44 | 6.37 | 8.67 | 11.48 | 1.06 | 8.24 | 14.06 | 15.98 |
| Atr | 8.05 ± | 10.08 ± | 14.07 ± | 1.01 ± | 27.07 ± | 14.58 ± | 16.47 ± | 1.15 ± |
| | 9.32 | 10.30 | 25.46 | 0.42 | 2.73 | 12.32 | 8.36 | 0.31 |
| Bag1 | 24.87 ± | 25.05 ± | 42.29 ± | 25.72 ± | 23.32 ± | 32.76 ± | 42.01 ± | 21.87 ± |
| | 1.31 | 6.93 | 2.57 | 18.16 | 5.99 | 3.53 | 3.63 | 19.52 |
| Bax | 1.97 ± | 1.60 ± | 23.39 ± | 20.93 ± | 1.68 ± | 1.61 ± | 30.76 ± | 21.38 ± |
| | 0.35 | 0.24 | 8.55 | 16.37 | 0.11 | 0.04 | 6.53 | 19.63 |
| Bbc3 | 2.97 ± | 1.80 ± | 1.32 ± | 1.08 ± | 1.97 ± | 2.27 ± | 1.04 ± | 1.10 ± |
| | 0.54 | 0.53 | 0.05 | 0.61 | 0.34 | 0.18 | 0.10 | 0.31 |
| Bcl2 | 7.84 ± | 30.58 ± | — | 2.76 ± | 28.71 ± | 20.93 ± | 23.30 ± | 6.74 ± |
| | 5.30 | 5.82 | | 37.32 | 6.93 | 8.21 | 4.87 | 10.99 |
| Bid | 1.19 ± | 1.04 ± | 1.21 ± | 1.12 ± | 12.20 ± | 1.08 ± | 1.01 ± | 2.75 ± |
| | 0.19 | 0.04 | 0.20 | 0.21 | 11.26 | 0.03 | 0.08 | 5.89 |
| Birc5 | 15.16 ± | 45.92 ± | 50.48 ± | 12.81 ± | 26.07 ± | 53.57 ± | 66.42 ± | 24.85 ± |
| | 5.76 | 18.70 | 11.71 | 38.47 | 1.26 | 11.72 | 10.53 | 21.69 |
| Bnip3 | 53.30 ± | 39.22 ± | 67.81 ± | 55.29 ± | 38.13 ± | 64.56 ± | 74.63 ± | 49.94 ± |
| | 11.57 | 16.04 | 8.08 | 17.29 | 36.79 | 1.47 | 1.87 | 43.00 |
| Brca1 | 3.14 ± | 42.43 ± | 31.46 ± | 10.57 ± | 34.27 ± | 44.99 ± | 60.55 ± | 17.44 ± |
| | 8.06 | 27.56 | 13.27 | 45.07 | 7.93 | 5.75 | 6.93 | 14.79 |
| Brca2 | 5.75 ± | 36.40 ± | 25.33 ± | 12.51 ± | 36.90 ± | 34.71 ± | 41.49 ± | 19.53 ± |
| | 16.05 | 19.57 | 8.21 | 36.21 | 3.84 | 4.42 | 15.58 | 19.06 |
| Btg2 | 14.84 ± | 31.06 ± | 11.23 ± | 16.69 ± | 13.04 ± | 1.06 ± | 1.05 ± | 23.10 ± |
| | 17.41 | 19.84 | 15.94 | 7.58 | 20.28 | 0.17 | 0.06 | 24.36 |
| Casp2 | 17.74 ± | 16.13 ± | 9.89 ± | 5.88 ± | 34.08 ± | 28.04 ± | 15.74 ± | 8.98 ± |
| | 7.91 | 9.72 | 2.48 | 25.27 | 3.52 | 5.31 | 7.85 | 14.90 |
| Casp9 | 1.02 ± | 1.22 ± | 1.04 ± | 9.97 ± | 13.31 ± | 1.07 ± | 1.80 ± | 5.61 ± |
| | 0.06 | 0.16 | 0.03 | 9.73 | 10.34 | 0.03 | 0.05 | 26.33 |
| Ccnb1 | 4.81 ± | 65.51 ± | 59.73 ± | 10.10 ± | 15.72 ± | 69.21 ± | 73.80 ± | 18.77 ± |
| | 9.78 | 9.70 | 10.38 | 35.96 | 6.24 | 3.86 | 7.74 | 20.83 |
| Ccne1 | 12.14 ± | 29.87 ± | 5.97 ± | 1.09 ± | 40.62 ± | 38.46 ± | 38.25 ± | 1.04 ± |
| | 6.49 | 23.17 | 5.91 | 0.33 | 5.29 | 5.17 | 15.82 | 0.04 |
| Ccng1 | 6.19 ± | 4.63 ± | 9.36 ± | 9.69 ± | 6.49 ± | 6.84 ± | 1.45 ± | 11.26 ± |
| | 1.38 | 1.08 | 6.15 | 16.33 | 0.68 | 0.44 | 0.17 | 12.22 |
| Ccnh | 15.28 ± | 3.31 ± | 12.98 ± | 11.39 ± | 29.12 ± | 16.52 ± | 16.38 ± | 5.78 ± |
| | 0.96 | 5.29 | 1.61 | 25.64 | 5.40 | 1.33 | 9.93 | 14.37 |
| Cdc25a | 15.20 ± | 34.15 ± | 16.12 ± | 7.34 ± | 34.26 ± | 41.75 ± | 42.22 ± | 1.65 ± |
| | 4.70 | 9.98 | 2.28 | 39.79 | 6.15 | 2.67 | 6.74 | 8.12 |
| Cdc25c | 9.12 ± | 60.54 ± | 57.26 ± | 6.34 ± | 11.85 ± | 67.11 ± | 69.05 ± | 20.29 ± |
| | 12.00 | 12.78 | 15.14 | 42.44 | 3.34 | 6.76 | 6.41 | 23.25 |
| Cdk1 | 32.00 ± | 59.92 ± | 55.02 ± | 23.09 ± | 39.81 ± | 64.58 ± | 69.40 ± | 22.76 ± |
| | 5.41 | 8.56 | 7.10 | 28.93 | 1.40 | 5.05 | 5.78 | 20.85 |
| Cdk4 | 37.50 ± | 40.50 ± | 48.77 ± | 22.60 ± | 47.61 ± | 55.89 ± | 54.23 ± | 19.73 ± |
| | 3.26 | 2.81 | 3.57 | 30.13 | 0.51 | 3.72 | 4.12 | 18.39 |
| Cdkn1a | 7.14 ± | 5.16 ± | 1.03 ± | 1.42 ± | 8.66 ± | 10.93 ± | 1.38 ± | 13.49 ± |
| | 1.49 | 2.96 | 0.22 | 35.39 | 0.84 | 1.00 | 0.19 | 13.30 |
| Cdkn2a | 31.09 ± | 16.83 ± | 23.77 ± | 39.53 ± | 42.49 ± | 36.93 ± | 38.18 ± | 20.75 ± |
| | 4.58 | 3.24 | 12.57 | 16.20 | 7.28 | 4.50 | 7.70 | 28.14 |
| Chek1 | 19.58 ± | 52.36 ± | 37.45 ± | 20.93 ± | 41.91 ± | 58.77 ± | 63.16 ± | 17.70 ± |
| | 8.04 | 13.84 | 7.40 | 30.32 | 7.72 | 4.40 | 6.44 | 17.91 |
| Chek2 | 1.23 ± | 10.21 ± | 1.03 ± | 15.97 ± | 29.57 ± | 25.29 ± | 44.35 ± | 8.78 ± |
| | 0.06 | 27.74 | 0.01 | 41.39 | 6.12 | 0.45 | 3.90 | 34.70 |
| Cradd | 21.01 ± | 5.90 ± | 13.67 ± | 31.95 ± | 36.95 ± | 18.77 ± | 24.24 ± | 16.72 ± |
| | 8.59 | 17.03 | 15.86 | 7.29 | 2.29 | 7.60 | 3.25 | 17.68 |
| Cul9 | 1.73 ± | 1.58 ± | 2.20 ± | 1.28 ± | 26.00 ± | 1.05 ± | 1.03 ± | 1.54 ± |
| | 0.42 | 0.67 | 0.28 | 0.68 | 9.80 | 15.19 | 0.21 | 0.68 |
| Dapk1 | 1.37 ± | 1.19 ± | 1.46 ± | 1.11 ± | 2.02 ± | 1.14 ± | 1.17 ± | 1.39 ± |
| | 0.20 | 0.27 | 0.24 | 0.14 | 11.17 | 0.06 | 0.06 | 0.41 |
| Dnmt1 | 1.16 ± | 17.60 ± | 1.11 ± | 1.02 ± | 17.37 ± | 22.85 ± | 34.04 ± | 1.03 ± |
| | 0.12 | 29.99 | 0.19 | 0.41 | 3.29 | 6.04 | 12.32 | 0.25 |
| E2f1 | 1.05 ± | 10.67 ± | 1.24 ± | 1.14 ± | 27.89 ± | 26.25 ± | 30.48 ± | 1.10 ± |
| | 0.06 | 33.43 | 0.04 | 0.62 | 3.11 | 3.95 | 13.00 | 0.33 |
| E2f3 | 21.77 ± | 27.77 ± | 30.44 ± | 15.87 ± | 27.45 ± | 39.40 ± | 33.97 ± | 17.09 ± |
| | 4.84 | 2.51 | 6.19 | 23.51 | 5.76 | 7.97 | 4.75 | 14.65 |
| Egfr | 42.31 ± | 28.18 ± | 34.80 ± | 24.18 ± | 39.84 ± | 47.37 ± | 41.85 ± | 13.15 ± |
| | 3.97 | 11.85 | 1.23 | 30.20 | 8.34 | 5.31 | 4.23 | 11.31 |
| Egr1 | 59.72 ± | 64.73 ± | 39.68 ± | 40.49 ± | 53.86 ± | 66.33 ± | 46.38 ± | 38.21 ± |
| | 1.22 | 14.38 | 9.02 | 19.43 | 8.25 | 5.76 | 16.27 | 32.91 |
| Ep300 | 15.26 ± | 3.09 ± | 1.01 ± | 9.15 ± | 11.02 ± | 1.15 ± | 1.10 ± | 2.74 ± |
| | 2.44 | 22.84 | 0.13 | 6.30 | 3.82 | 0.14 | 0.09 | 12.08 |
| Ercc1 | 20.74 ± | 18.66 ± | 43.22 ± | 29.53 ± | 16.08 ± | 34.86 ± | 44.10 ± | 20.27 ± |
| | 3.05 | 5.43 | 2.16 | 11.01 | 6.73 | 4.52 | 7.53 | 18.48 |
| Esr1 | 1.22 ± | 1.26 ± | 1.78 ± | 1.39 ± | 14.42 ± | 1.24 ± | 1.67 ± | 1.82 ± |
| | 0.16 | 0.03 | 0.14 | 0.28 | 4.49 | 0.10 | 0.03 | 0.56 |
| Fadd | 2.66 ± | 5.60 ± | 1.13 ± | 1.43 ± | 31.64 ± | 8.43 ± | 9.11 ± | 1.18 ± |
| | 8.65 | 11.01 | 0.02 | 28.59 | 6.05 | 1.87 | 1.89 | 0.20 |
| Fas | 1.75 ± | 1.45 ± | 10.67 ± | 12.05 ± | 2.12 ± | 1.94 ± | 10.02 ± | 12.10 ± |
| | 0.45 | 0.21 | 13.71 | 5.81 | 0.50 | 0.04 | 22.86 | 16.64 |
| Fasl | 1.24 ± | 10.36 ± | 1.48 ± | 1.42 ± | 3.98 ± | 6.77 ± | 1.18 ± | 1.29 ± |
| | 0.57 | 16.61 | 0.65 | 0.22 | 32.57 | 10.33 | 0.30 | 0.47 |
| Foxo3 | 1.04 ± | 3.58 ± | 3.18 ± | 14.18 ± | 1.15 ± | 1.20 ± | 7.35 ± | 1.08 ± |
| | 0.14 | 15.94 | 16.97 | 10.26 | 0.05 | 0.10 | 4.65 | 0.18 |
| Gadd45a | 25.34 ± | 19.41 ± | 49.07 ± | 25.11 ± | 23.94 ± | 38.66 ± | 42.12 ± | 12.31 ± |
| | 3.40 | 8.50 | 4.58 | 34.35 | 3.97 | 11.13 | 13.70 | 10.55 |
| Hif1a | 1.26 ± | 13.43 ± | 1.22 ± | 1.19 ± | 1.07 ± | 10.42 ± | 2.87 ± | 1.18 ± |
| | 0.08 | 9.04 | 0.15 | 0.17 | 0.14 | 9.43 | 7.20 | 0.18 |
| Il6 | 68.47 ± | 70.78 ± | 80.18 ± | 65.67 ± | 42.72 ± | 85.31 ± | 79.27 ± | 61.14 ± |
| | 3.89 | 12.10 | 11.73 | 38.69 | 43.60 | 0.49 | 7.77 | 52.49 |
| Jun | 22.75 ± | 38.98 ± | 29.72 ± | 25.77 ± | 32.68 ± | 46.70 ± | 47.43 ± | 20.41 ± |
| | 1.69 | 2.29 | 7.11 | 20.53 | 4.14 | 6.13 | 6.36 | 17.90 |
| Kras | 1.03 ± | 13.87 ± | 1.01 ± | 1.09 ± | 8.77 ± | 9.19 ± | 1.18 ± | 1.08 ± |
| | 0.04 | 8.57 | 0.02 | 0.28 | 1.73 | 5.22 | 4.33 | 0.09 |
| Lig4 | 1.13 ± | 1.17. ± | 1.05 ± | 1.12 ± | 9.59 ± | 1.02 ± | 1.12 ± | 1.27 ± |
| | 0.03 | 0.23 | 0.18 | 0.34 | 8.06 | 0.11 | 0.06 | 0.28 |
| Mel1 | 24.40 ± | 15.14 ± | 31.09 ± | 21.11 ± | 3.87 ± | 9.59 ± | 6.76 ± | 17.68 ± |
| | 5.43 | 17.92 | 1.21 | 4.95 | 17.85 | 5.71 | 2.77 | 15.40 |
| Mdm2 | 5.35 ± | 2.76 ± | 12.55 ± | 2.80 ± | 7.29 ± | 5.96 ± | 1.03 ± | 1.10 ± |
| | 1.95 | 1.41 | 10.47 | 24.46 | 1.71 | 0.91 | 0.08 | 0.24 |
| Mdm4 | 1.03 ± | 1.07 ± | 19.35 ± | 14.78 ± | 0.59 ± | 1.22 ± | 1.28 ± | 13.89 ± |
| | 0.09 | 0.15 | 7.02 | 24.14 | 17.53 | 0.14 | 20.15 | 15.08 |
| Mlh1 | 12.48 ± | 9.12 ± | 7.78 ± | 4.80 ± | 28.64 ± | 10.59 ± | 19.02 ± | 1.52 ± |
| | 2.32 | 5.84 | 2.99 | 37.43 | 5.13 | 6.44 | 11.92 | 17.73 |
| Msh2 | 31.49 ± | 29.40 ± | 11.63 ± | 16.07 ± | 35.13 ± | 26.97 ± | 29.32 ± | 4.98 ± |
| | 6.10 | 6.77 | 6.08 | 21.53 | 3.83 | 2.63 | 2.53 | 8.69 |
| Myc | 24.92 ± | 38.12 ± | 26.10 ± | 20.11 ± | 27.70 ± | 46.55 ± | 36.54 ± | 13.27 ± |
| | 5.48 | 2.59 | 2.97 | 21.11 | 3.91 | 4.76 | 3.92 | 11.73 |
| Myod1 | 4.56 ± | 3.54 ± | 4.06 ± | 2.73 ± | 14.90 ± | 3.09 ± | 1.51 ± | 3.03 ± |
| | 1.84 | 2.40 | 1.51 | 2.78 | 16.04 | 0.00 | 0.11 | 3.34 |
| Nf1 | 31.85 ± | 16.46 ± | 29.60 ± | 21.43 ± | 17.13 ± | 12.02 ± | 9.92 ± | 12.40 ± |
| | 7.75 | 12.06 | 3.19 | 5.97 | 13.17 | 6.51 | 3.24 | 11.61 |
| Nfkb1 | 11.36 ± | 15.21 ± | 7.16 ± | 1.73 ± | 6.02 ± | 5.42 ± | 4.37 ± | 1.15 ± |
| | 6.56 | 5.42 | 1.74 | 17.58 | 7.61 | 11.26 | 1.69 | 0.10 |
| Nfkb1 | 11.36 ± | 15.21 ± | 7.16 ± | 1.73 ± | 6.02 ± | 5.42 ± | 4.37 ± | 1.15 ± |
| | 6.56 | 5.42 | 1.74 | 17.58 | 7.61 | 11.26 | 1.69 | 0.10 |
| Pcna | 32.47 ± | 43.21 ± | 38.45 ± | 23.22 ± | 44.75 ± | 42.63 ± | 58.04 ± | 21.44 ± |
| | 9.53 | 13.45 | 2.10 | 27.14 | 7.78 | 10.49 | 2.38 | 18.24 |
| Pmaip1 | 1.60 ± | 3.81 ± | 1.15 ± | 1.03 ± | 2.33 ± | 1.45 ± | 1.17 ± | 1.41 ± |
| | 0.38 | 3.48 | 0.26 | 0.13 | 0.67 | 0.12 | 0.3 | 0.53 |
| Ppm1d | 1.48 ± | 1.26 ± | 4.68 ± | 5.34 ± | 1.44 ± | 1.49 ± | 2.87 ± | 0.20 ± |
| | 0.22 | 0.21 | 9.55 | 29.53 | 0.21 | 0.14 | 13.63 | 15.01 |
| Nfkb1 | 11.36 ± | 15.21 ± | 7.16 ± | 1.73 ± | 6.02 ± | 5.42 ± | 4.37 ± | 1.15 ± |
| | 6.56 | 5.42 | 1.74 | 17.58 | 7.61 | 11.26 | 1.69 | 0.10 |
| Pcna | 32.47 ± | 43.21 ± | 38.45 ± | 23.22 ± | 44.75 ± | 42.63 ± | 58.04 ± | 21.44 ± |
| | 9.53 | 13.45 | 2.10 | 27.14 | 7.78 | 10.49 | 2.38 | 18.24 |
| Pmaip1 | 1.60 ± | 3.81 ± | 1.15 ± | 1.03 ± | 2.33 ± | 1.45 ± | 1.17 ± | 1.41 ± |
| | 0.38 | 3.48 | 0.26 | 0.13 | 0.67 | 0.12 | 0.3 | 0.53 |
| Ppm1d | 1.48 ± | 1.26 ± | 4.68 ± | 5.34 ± | 1.44 ± | 1.49 ± | 2.87 ± | 0.20 ± |
| | 0.22 | 0.21 | 9.55 | 29.53 | 0.21 | 0.14 | 13.63 | 15.01 |
| Prc1 | 1.28 ± | 26.03 ± | 16.43 ± | 6.02 ± | 17.14 ± | 54.87 ± | 59.70 ± | — |
| | 0.03 | 29.24 | 11.23 | 41.02 | 8.35 | 3.51 | 5.90 | |
| Prkca | 5.77 ± | 16.45 ± | 13.73 ± | 6.98 ± | 27.03 ± | 28.12 ± | 22.18 ± | 4.15 ± |
| | 13.57 | 7.60 | 10.67 | 34.36 | 3.75 | 3.39 | 6.00 | 10.25 |
| Pten | 0.35 ± | 1.18 ± | 1.01 ± | 3.49 ± | 14.47 ± | 1.08 ± | 1.08 ± | 1.14 ± |
| | 4.49 | 0.12 | 0.11 | 19.03 | 7.64 | 0.03 | 0.09 | 0.27 |
| Pttg1 | 1.04 ± | 1.07 ± | 1.08 ± | 1.03 ± | 3.91 ± | 1.11 ± | 0.69 ± | 1.19 ± |
| | 0.01 | 0.19 | 0.07 | 0.26 | 5.89 | 0.14 | 5.22 | 0.19 |
| Rb1 | 16.87 ± | 1.14 ± | 2.55 ± | 3.16 ± | 1.08 ± | 1.48 ± | 1.43 ± | 3.47 ± |
| | 6.35 | 0.22 | 0.61 | 4.36 | 0.20 | 0.16 | 0.19 | 9.76 |
| Rela | 1.12 ± | 32.18 ± | 1.07 ± | 4.69 ± | 5.08 ± | 14.73 ± | 9.46 ± | 2.50 ± |
| | 0.16 | 5.35 | 0.15 | 14.39 | 8.67 | 14.51 | 11.39 | 3.01 |
| Rprm | 3.08 ± | 1.77 ± | 2.09 ± | 1.34 ± | 1.81 ± | 2.12 ± | 1.00 ± | 1.51 ± |
| | 0.80 | 0.30 | 0.40 | 0.69 | 0.18 | 0.23 | 0.20 | 0.45 |
| Serpinb5 | 3.96 ± | 1.20 ± | 1.90 ± | 15.52 ± | 2.43 ± | 2.23 ± | 3.17 ± | 1.00 ± |
| | 2.23 | 0.23 | 0.80 | 13.95 | 1.35 | 1.14 | 5.48 | 0.00 |
| Sesn2 | 3.24 ± | 2.05 ± | 1.57 ± | 1.11 ± | 2.88 ± | 2.45 ± | 1.21 ± | 1.31 ± |
| | 0.28 | 0.48 | 0.21 | 0.36 | 0.18 | 0.19 | 0.10 | 0.46 |
| Sfn | 1.12 ± | 15.25 ± | 1.00 ± | 8.30 ± | 15.09 ± | 31.21 ± | 13.65 ± | 7.33 ± |
| | 0.21 | 15.05 | 0.07 | 14.44 | 7.17 | 8.79 | 6.82 | 16.47 |
| Sirt1 | 1.09 ± | 6.13 ± | 1.01 ± | 5.18 ± | 24.95 ± | 15.06 ± | 13.36 ± | 1.08 ± |
| | 0.07 | 12.57 | 0.11 | 39.81 | 11.58 | 5.19 | 12.83 | 0.19 |
| Stat1 | 1.35 ± | 1.08 ± | 1.73 ± | 1.58 ± | 1.23 ± | 1.51 ± | 1.72 ± | 1.78 ± |
| | 0.35 | 0.10 | 0.34 | 0.38 | 0.19 | 0.18 | 0.19 | 0.45 |
| Prkca | 5.77 ± | 16.45 ± | 13.73 ± | 6.98 ± | 27.03 ± | 28.12 ± | 22.18 ± | 4.15 ± |
| | 13.57 | 7.60 | 10.67 | 34.36 | 3.75 | 3.39 | 6.00 | 10.25 |
| Pten | 0.35 ± | 1.18 ± | 1.01 ± | 3.49 ± | 14.47 ± | 1.08 ± | 1.08 ± | 1.14 ± |
| | 4.49 | 0.12 | 0.11 | 19.03 | 7.64 | 0.03 | 0.09 | 0.27 |
| Pttg1 | 1.04 ± | 1.07 ± | 1.08 ± | 1.03 ± | 3.91 ± | 1.11 ± | 0.69 ± | 1.19 ± |
| | 0.01 | 0.19 | 0.07 | 0.26 | 5.89 | 0.14 | 5.22 | 0.19 |
| Rb1 | 16.87 ± | 1.14 ± | 2.55 ± | 3.16 ± | 1.08 ± | 1.48 ± | 1.43 ± | 3.47 ± |
| | 6.35 | 0.22 | 0.61 | 4.36 | 0.20 | 0.16 | 0.19 | 9.76 |
| Rela | 1.12 ± | 32.18 ± | 1.07 ± | 4.69 ± | 5.08 ± | 14.73 ± | 9.46 ± | 2.50 ± |
| | 0.16 | 5.35 | 0.15 | 14.39 | 8.67 | 14.51 | 11.39 | 3.01 |
| Rprm | 3.08 ± | 1.77 ± | 2.09 ± | 1.34 ± | 1.81 ± | 2.12 ± | 1.00 ± | 1.51 ± |
| | 0.80 | 0.30 | 0.40 | 0.69 | 0.18 | 0.23 | 0.20 | 0.45 |
| Serpinb5 | 3.96 ± | 1.20 ± | 1.90 ± | 15.52 ± | 2.43 ± | 2.23 ± | 3.17 ± | 1.00 ± |
| | 2.23 | 0.23 | 0.80 | 13.95 | 1.35 | 1.14 | 5.48 | 0.00 |
| Sesn2 | 3.24 ± | 2.05 ± | 1.57 ± | 1.11 ± | 2.88 ± | 2.45 ± | 1.21 ± | 1.31 ± |
| | 0.28 | 0.48 | 0.21 | 0.36 | 0.18 | 0.19 | 0.10 | 0.46 |
| Sfn | 1.12 ± | 15.25 ± | 1.00 ± | 8.30 ± | 15.09 ± | 31.21 ± | 13.65 ± | 7.33 ± |
| | 0.21 | 15.05 | 0.07 | 14.44 | 7.17 | 8.79 | 6.82 | 16.47 |
| Sirt1 | 1.09 ± | 6.13 ± | 1.01 ± | 5.18 ± | 24.95 ± | 15.06 ± | 13.36 ± | 1.08 ± |
| | 0.07 | 12.57 | 0.11 | 39.81 | 11.58 | 5.19 | 12.83 | 0.19 |
| Stat1 | 1.35 ± | 1.08 ± | 1.73 ± | 1.58 ± | 1.23 ± | 1.51 ± | 1.72 ± | 1.78 ± |
| | 0.35 | 0.10 | 0.34 | 0.38 | 0.19 | 0.18 | 0.19 | 0.45 |
| Tnf | 1.85 ± | 24.76 ± | 1.36 ± | 1.28 ± | 1.57 ± | 1.23 ± | 27.80 ± | 1.15 ± |
| | 0.78 | 19.32 | 0.75 | 0.26 | 0.60 | 0.50 | 3.95 | 0.25 |
| Tnfrsf10b | 2.12 ± | 1.31 ± | 13.46 ± | 0.90 ± | 2.96 ± | 2.29 ± | 3.55 ± | 1.09 ± |
| | 0.41 | 0.73 | 8.98 | 12.32 | 0.24 | 0.21 | 7.07 | 0.19 |
| Traf1 | 1.23 ± | 1.06 ± | 1.38 ± | 1.16 ± | 1.4 ± | 1.20 ± | 1.04 ± | 1.41 ± |
| | 0.03 | 0.15 | 0.22 | 0.26 | 0.17 | 0.18 | 0.10 | 0.31 |
| Trp53 | 1.13 ± | 18.16 ± | 1.16 ± | 1.11 ± | 16.49 ± | 27.25 ± | 22.40 ± | 1.21 ± |
| | 0.06 | 14.32 | 0.10 | 0.38 | 9.17 | 4.41 | 5.18 | 0.26 |
| Trp53bp2 | 12.87 ± | 5.61 ± | 1.06 ± | 7.94 ± | 29.54 ± | 12.94 ± | 8.14 ± | 1.07 ± |
| | 7.67 | 7.82 | 0.13 | 31.51 | 6.76 | 3.32 | 6.09 | 0.16 |
| Trp63 | 3.58 ± | 1.58 ± | 2.65 ± | 1.62 ± | 1.02 ± | 1.00 ± | 1.00 ± | 1.67 ± |
| | 2.53 | 1.00 | 1.06 | 1.22 | 0.04 | 0.00 | 0.00 | 1.16 |
| Trp73 | 16.09 ± | 1.01 ± | 3.40 ± | 1.00 ± | 1.00 ± | 1.00 ± | 1.00 ± | 1.00 ± |
| | 12.16 | 0.01 | 16.48 | 0.00 | 0.00 | 0.00 | 0.00 | 0.00 |
| Wt1 | 28.78 ± | 21.25 ± | 21.51 ± | 23.33 ± | 30.29 ± | 21.51 ± | 21.51 ± | 21.51 ± |
| | 12.60 | 0.44 | 0.00 | 3.15 | 15.21 | 0 | 0.00 | 0.00 |
| Xrcc4 | 24.65 ± | 1.06 ± | 26.47 ± | 21.24 ± | 33.04 ± | 24.37 ± | 24.99 ± | 16.19 ± |
| | 3.55 | 0.12 | 6.66 | 25.82 | 11.74 | 3.37 | 10.99 | 19.05 |
| Xrcc5 | 39.69 ± | 21.81 ± | 35.55 ± | 22.62 ± | 49.00 ± | 35.69 ± | 32.10 ± | 16.13 ± |
| | 9.75 | 8.03 | 12.95 | 25.44 | 2.47 | 2.04 | 5.16 | 15.69 |
| Zmat3 | 6.25 ± | 4.14 ± | 1.02 ± | 1.10 ± | 6.69 ± | 6.17 ± | 1.36 ± | 1.21 ± |
| | 0.56 | 1.64 | 0.06 | 0.28 | 0.94 | 0.36 | 0.22 | 0.03 |
| Actb* | 1.60 ± | 1.08 ± | 1.07 ± | 1.06 ± | 1.00 ± | 1.11 ± | 1.04 ± | 1.04 ± |
| | 7.32 | 9.33 | 0.09 | 0.03 | 0.11 | 0.02 | 0.11 | 0.08 |
| B2m* | 1.16 ± | 1.14 ± | 1.27 ± | 1.11 ± | 1.05 ± | 1.11 ± | 1.20 ± | 1.18 ± |
| | 0.20 | 0.09 | 0.19 | 0.03 | 0.09 | 0.02 | 0.16 | 0.17 |
| Gapdh* | 11.24 ± | 13.68 ± | 34.54 ± | 14.41 ± | 1.02 ± | 14.16 ± | 27.30 ± | 19.69 ± |
| | 15.54 | 4.09 | 4.85 | 20.26 | 0.03 | 2.50 | 3.83 | 21.78 |
| Gusb* | 1.12 ± | 1.25 ± | 1.31 ± | 1.11 ± | 1.02 ± | 1.25 ± | 1.39 ± | 1.27 ± |
| | 0.02 | 0.09 | 0.08 | 0.12 | 0.05 | 0.01 | 0.05 | 0.20 |
| Hsp90ab1* | 10.06 ± | 17.39 ± | 12.45 ± | 7.83 ± | 9.13 ± | 24.42 ± | 19.96 ± | 1.00 ± |
| | 7.15 | 8.09 | 5.82 | 13.19 | 4.50 | 0.60 | 0.91 | 1.09 |
| MGDC* | 1.79 ± | 1.27 ± | 1.78 ± | 1.36 ± | 1.00 ± | 1.00 ± | 1.00 ± | 1.53 ± |
| | 0.23 | 0.47 | 0.18 | 0.62 | 0.00 | 0.00 | 0.00 | 0.91 |

*Housekeeping or reference gene

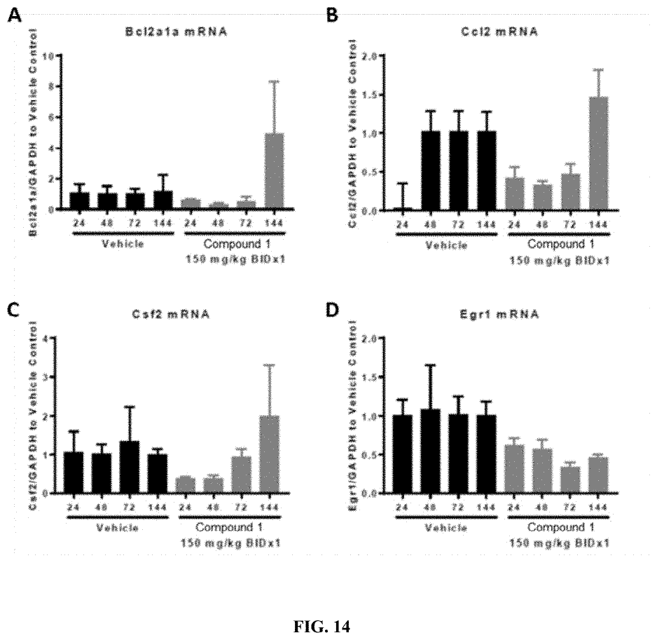

p53 plays important roles in the NF-κB pathway. Measurement of NF-κB pathway transcripts both downstream and upstream of NF-κB were investigated at only the higher dose level (Compound 1 150 mg/kg BID×1) and at selected timepoints (24, 48, 72, and 144 h) to understand changes in the NF-κB pathway with reactivation of WT p53. TABLE 20 and FIG. 14 PANEL A-PANEL D show the fold change increase and percent reduction of various genes relative to the vehicle control at different timepoints. Several genes were downregulated at various timepoints that were related to an immune response: Ccl2 (57.69% at 24 h, 67.28% at 48 h, 52.53% at 72 h), Csf2 (61.29% at 24 h, 62.24% at 48 h), Ifnγ (35.82% at 24 h, 43.46% at 48 h, 22.04% at 72 h), Il1α (13.50% at 24 h, 28.88% at 48 h, 63.60% at 72 h), Il1β (63.25% at 24 h, 18.73% at 48 h, 84.09% at 72 h). Egfr, a gene involved in proliferation, was downregulated at various time points (28.45% at 24 h, 24.42% at 48 h, 41.90% at 144 h). Fas1, a gene involved in apoptosis, was also downregulated at several timepoints (31.85% at 24 h, 42.85% at 72 h).

TABLE 20
Compound 1 at 150 mg/kg BIDx1
Fold Increase Over Vehicle Control or Percent
Reduction Compared to Vehicle Control (%) ± StDev

| Gene | 24 h | 48 h | 72 h | 144 h |
|---|---|---|---|---|
| Agt | 8.18 ± 30.61 | 1.60 ± 0.41 | 2.05 ± 0.66 | 50.51 ± 41.92 |
| Akt1 | 17.84 ± 6.63 | 4.50 ± 1.92 | 1.06 ± 0.07 | 8.16 ± 7.88 |
| Atf1 | 42.27 ± 2.47 | 42.46 ± 1.99 | 34.00 ± 7.86 | 1.13 ± 0.30 |
| Atf2 | 24.19 ± 2.49 | 30.02 ± 3.86 | 8.53 ± 13.61 | 1.00 ± 0.20 |
| Bcl10 | 38.94 ± 3.05 | 36.06 ± 3.54 | 23.25 ± 7.71 | 1.18 ± 0.09 |
| Bcl2a1a | 39.52 ± 5.46 | 67.64 ± 10.73 | 48.45 ± 28.65 | 4.90 ± 3.38 |
| Bcl2l1 | 1.43 ± 0.11 | 7.49 ± 1.18 | 1.06 ± 0.18 | 1.00 ± 0.07 |
| Bcl3 | 1.29 ± 0.31 | 1.35 ± 0.16 | 1.22 ± 0.24 | 14.36 ± 16.67 |
| Birc3 | 1.03 ± 0.07 | 1.20 ± 0.28 | 1.13 ± 0.28 | 1.10 ± 0.26 |
| Card10 | 1.32 ± 0.33 | 1.20 ± 0.11 | 1.39 ± 0.49 | 40.14 ± 8.96 |
| Card11 | 1.14 ± 0.14 | 1.67 ± 0.42 | 1.52 ± 0.54 | 1.85 ± 0.30 |
| Casp1 | 14.78 ± 4.65 | 10.29 ± 10.66 | 9.73 ± 31.75 | 1.82 ± 0.71 |
| Casp8 | 9.24 ± 3.65 | 1.02 ± 0.01 | 1.04 ± 0.13 | 1.14 ± 0.07 |
| Ccl2 | 57.69 ± 14.13 | 67.28 ± 5.26 | 52.53 ± 13.14 | 1.47 ± 0.35 |
| Ccl5 | 21.82 ± 7.86 | 8.18 ± 19.38 | 2.54 ± 37.88 | 2.85 ± 0.20 |
| Cd27 | 1.11 ± 0.11 | 1.46 ± 0.26 | 1.53 ± 0.39 | 1.48 ± 0.16 |
| Cd40 | 30.05 ± 5.34 | 39.20 ± 14.39 | 20.33 ± 32.50 | 1.78 ± 0.80 |
| Cflar | 1.01 ± 0.08 | 7.92 ± 8.21 | 1.10 ± 0.20 | 6.22 ± 11.55 |
| Chuk | 1.03 ± 0.07 | 4.04 ± 12.31 | 5.62 ± 25.53 | 1.16 ± 0.33 |
| Crebbp | 1.07 ± 0.06 | 1.12 ± 0.05 | 1.12 ± 0.15 | 16.61 ± 5.43 |
| Csf1 | 14.27 ± 12.28 | 11.36 ± 7.30 | 3.08 ± 4.37 | 1.10 ± 0.10 |
| Csf2 | 61.29 ± 2.87 | 62.24 ± 8.70 | 5.23 ± 19.46 | 2.00 ± 1.31 |
| Csf3 | 27.25 ± 12.00 | 0.37 ± 11.46 | 46.68 ± 13.40 | 52.98 ± 22.53 |
| Egfr | 28.45 ± 5.44 | 24.42 ± 4.76 | 1.01 ± 0.23 | 41.90 ± 8.16 |
| Egr1 | 37.93 ± 8.71 | 42.64 ± 12.12 | 66.09 ± 6.29 | 54.35 ± 3.91 |
| Eif2ak2 | 1.05 ± 0.10 | 1.01 ± 0.03 | 5.36 ± 11.08 | 18.72 ± 7.21 |
| Elk1 | 12.50 ± 9.71 | 11.87 ± 8.21 | 19.15 ± 11.29 | 7.14 ± 8.40 |
| F2r | 40.30 ± 2.19 | 19.79 ± 4.23 | 14.84 ± 13.77 | 1.01 ± 0.12 |
| Fadd | 5.03 ± 3.48 | 7.23 ± 4.56 | 1.25 ± 0.13 | 5.68 ± 5.62 |
| Fasl | 31.85 ± 3.66 | 5.96 ± 19.38 | 42.85 ± 19.54 | 3.17 ± 0.74 |
| Fos | 12.09 ± 14.97 | 47.30 ± 7.08 | 86.19 ± 1.00 | 44.44 ± 21.93 |
| Hmox1 | 13.99 ± 4.70 | 27.03 ± 7.50 | 1.03 ± 0.18 | 40.88 ± 33.56 |
| Icam1 | 1.20 ± 0.24 | 3.83 ± 17.26 | 1.07 ± 0.18 | 1.41 ± 0.25 |
| Ifng | 35.82 ± 11.11 | 43.46 ± 15.02 | 22.04 ± 33.20 | 3.22 ± 0.99 |
| Ikbkb | 1.13 ± 0.20 | 1.24 ± 0.09 | 1.24 ± 0.27 | 6.97 ± 8.47 |
| Ikbke | 1.48 ± 0.37 | 1.71 ± 28.42 | 2.05 ± 40.34 | 0.36 ± 10.26 |
| Ikbkg | 1.07 ± 0.09 | 3.73 ± 5.88 | 1.06 ± 0.32 | 9.50 ± 15.53 |
| Il10 | 1.52 ± 0.15 | 1.15 ± 0.09 | 12.42 ± 41.31 | 34.39 ± 10.47 |
| Il1a | 13.50 ± 23.10 | 28.88 ± 7.08 | 63.60 ± 11.55 | 1.02 ± 0.18 |
| Il1b | 63.25 ± 18.78 | 18.73 ± 5.15 | 84.09 ± 6.24 | 1.39 ± 0.34 |
| Il1r1 | 31.14 ± 6.16 | 22.37 ± 5.01 | 9.26 ± 29.81 | 28.14 ± 8.59 |
| Irak1 | 1.93 ± 9.47 | 1.00 ± 2.36 | 4.37 ± 14.84 | 5.30 ± 10.81 |
| Irak2 | 24.86 ± 5.53 | 17.08 ± 1.54 | 1.07 ± 0.14 | 1.05 ± 0.12 |
| Irf1 | 4.44 ± 20.95 | 7.15 ± 7.58 | 1.06 ± 0.20 | 1.11 ± 0.14 |
| Jun | 29.31 ± 10.13 | 44.92 ± 3.26 | 26.30 ± 10.41 | 26.73 ± 6.02 |
| Lta | 12.59 ± 29.28 | 57.16 ± 12.86 | 26.12 ± 35.38 | 1.87 ± 0.82 |
| Ltbr | 11.61 ± 7.41 | 13.21 ± 2.85 | 1.05 ± 0.08 | 1.13 ± 0.10 |
| Map3k1 | 1.40 ± 0.14 | 1.74 ± 0.24 | 1.25 ± 0.28 | 16.75 ± 8.45 |
| Mapk3 | 10.05 ± 3.02 | 3.13 ± 7.94 | 1.22 ± 0.17 | 19.93 ± 6.52 |
| Myd88 | 1.33 ± 0.11 | 1.17 ± 0.06 | 37.76 ± 10.69 | 9.52 ± 8.80 |
| Nfkb1 | 1.39 ± 12.08 | 1.04 ± 0.04 | 1.06 ± 0.05 | 5.72 ± 2.88 |
| Nfkb2 | 1.01 ± 0.29 | 1.17 ± 0.11 | 1.30 ± 0.23 | 1.01 ± 0.10 |
| Nfkbia | 3.07 ± 2.68 | 1.14 ± 0.15 | 6.09 ± 32.11 | 8.21 ± 16.85 |
| Nod1 | 1.24 ± 0.24 | 1.49 ± 0.08 | 1.26 ± 0.26 | 0.36 ± 14.65 |
| Raf1 | 11.54 ± 3.56 | 0.48 ± 5.31 | 4.12 ± 9.21 | 7.66 ± 4.64 |
| Rel | 1.09 ± 0.06 | 1.30 ± 0.16 | 1.09 ± 0.23 | 4.74 ± 2.26 |
| Rela | 1.02 ± 0.07 | 1.07 ± 0.01 | 1.01 ± 0.08 | 20.75 ± 9.37 |
| Relb | 1.10 ± 0.14 | 1.09 ± 0.07 | 3.68 ± 22.32 | 14.05 ± 6.91 |
| Ripk1 | 1.25 ± 0.04 | 1.19 ± 0.07 | 1.11 ± 0.09 | 2.92 ± 15.10 |
| Ripk2 | 27.80 ± 7.83 | 24.29 ± 6.23 | 29.79 ± 5.69 | 1.11 ± 0.29 |
| Slc20a1 | 29.02 ± 4.69 | 40.28 ± 6.82 | 36.31 ± 7.52 | 20.53 ± 3.61 |
| Smad3 | 4.92 ± 19.78 | 1.04 ± 0.08 | 1.09 ± 0.18 | 0.01 ± 11.30 |
| Stat1 | 1.17 ± 0.19 | 1.36 ± 0.15 | 1.20 ± 0.20 | 1.20 ± 0.33 |
| Tbk1 | 6.71 ± 4.73 | 1.01 ± 6.05 | 1.17 ± 0.15 | 1.03 ± 0.18 |
| Tlr1 | 1.17 ± 0.16 | 1.63 ± 0.28 | 1.52 ± 0.56 | 1.33 ± 0.37 |
| Tlr2 | 4.13 ± 15.48 | 1.24 ± 0.15 | 1.05 ± 0.16 | 1.05 ± 0.08 |
| Tlr3 | 1.08 ± 0.12 | 1.00 ± 1.44 | 5.46 ± 14.97 | 1.02 ± 0.15 |
| Tlr4 | 1.11 ± 0.05 | 6.31 ± 9.66 | 1.13 ± 0.27 | 1.04 ± 0.09 |
| Tlr6 | 1.04 ± 0.10 | 1.33 ± 0.08 | 1.25 ± 0.31 | 0.43 ± 6.189 |
| Tlr9 | 1.33 ± 0.19 | 1.92 ± 0.29 | 1.78 ± 0.32 | 1.29 ± 0.19 |
| Tnf | 1.84 ± 47.89 | 56.86 ± 13.49 | 3.41 ± 24.15 | 1.12 ± 0.27 |
| Tnfaip3 | 3.01 ± 14.38 | 1.26 ± 0.33 | 10.12 ± 40.91 | 11.08 ± 10.07 |
| Tnfrsf10b | 2.61 ± 0.31 | 11.34 ± 5.36 | 1.17 ± 0.22 | 1.19 ± 15.33 |
| Tnfrsf1a | 1.21 ± 0.13 | 1.35 ± 0.08 | 1.18 ± 0.13 | 17.69 ± 12.43 |
| Tnfrsf1b | 1.23 ± 0.12 | 1.66 ± 0.08 | 1.52 ± 0.29 | 1.08 ± 0.19 |
| Tnfsf10 | 18.51 ± 14.05 | 16.58 ± 3.82 | 1.09 ± 0.21 | 1.18 ± 0.41 |
| Tnfsf14 | 1.11 ± 0.17 | 1.53 ± 0.40 | 1.34 ± 0.30 | 1.09 ± 0.34 |
| Tollip | 1.01 ± 0.06 | 2.45 ± 1.53 | 1.11 ± 0.10 | 13.73 ± 7.04 |
| Tradd | 1.17 ± 0.08 | 1.01 ± 0.06 | 1.10 ± 0.24 | 1.02 ± 0.22 |
| Traf2 | 8.21 ± 7.59 | 7.23 ± 7.66 | 1.17 ± 0.13 | 1.01 ± 0.11 |
| Traf3 | 13.02 ± 10.57 | 13.20 ± 10.35 | 3.24 ± 6.50 | 17.66 ± 4.06 |
| Traf5 | 21.79 ± 2.60 | 2.99 ± 3.31 | 1.24 ± 0.14 | 1.10 ± 0.12 |
| Traf6 | 1.03 ± 0.16 | 1.07 ± 0.12 | 1.17 ± 0.24 | 12.35 ± 5.34 |
| Zap70 | 1.01 ± 0.07 | 1.45 ± 0.41 | 1.52 ± 0.48 | 2.40 ± 0.39 |

Conclusions: The PK and PD relationship of Compound 1 was tested in a mouse syngeneic model of sarcoma (MT373). Compound 1 was dosed PO at 75 and 150 mg/kg BID×1. At termination of the study tumor and plasma were collected for PD/PK analysis. Plasma concentrations were in the expected range for the dose levels at the various timepoints. Mice treated with 75 and 150 mg/kg BID×1 were harvested 8, 24, 48, 72, 96, and 144 h post the first dose. The tumors were measured to have a dose responsive decrease in mutant p53 (53.91% and 82.30% at 8 h, respectively) and a dose responsive increase in WT conformation p53 levels (1.25 and 1.45-fold over vehicle control at 8 h, respectively). The observed changes in p53 conformation were consistent with high plasma concentrations at Cₘₐₓ (8375 and 11435 ng/mL). The PD between the two doses was dose-responsive with the lower dose having a decreased and more transitory PD effect than at the higher dose.

Analysis of downstream p53 transcriptional targets p21, MDM2, and MIC-1 showed a dose responsive increase in p21 (5.4 and 6.7-fold) and MDM2 (6.2 and 8.7-fold) mRNA at 8 h post-first dose for the 75 and 150 mg/kg dose groups, respectively. MIC1 mRNA levels only increased in the 150 mg/kg group with a 3.07-fold over vehicle change at 8 h. To understand further changes associated with reactivation of p53, 84 genes that are upstream or downstream of the p53 pathway were analyzed. For genes with at least a 2-fold increase or 50% decrease in expression, a minimal dose-response between 75 and 150 mg/kg was observed. Maximal changes at the various timepoints were gene specific, for instance, 116 downregulation at both dose levels was maximal at 24, 48, and 72 h, but maximal changes for Cdkn1a were at 8 and 24 h. These p53 related genes have functions in regulation of apoptosis (Bax), cell cycle control (Ccng1, Cdc25c, Cdk1, Cdkn1a, Chek1, Zmat1), cell growth and proliferation (Egfr, Sesn2), ubiquitination (Mdm2), and inflammation and immune response (116). A smaller subset of the PD samples, tumors treated with Compound 1 150 mg/kg BID×1 at 24, 48, 72, and 144 h, were analyzed for NF-κB pathway gene expression. The genes with at least a 2-fold increase or 50% decrease in expression compared to vehicle had functions in apoptosis (Bcl2a1a), immune system regulation (Ccl2 and Csf2), and cell proliferation (Egr1).

Overall, administration of Compound 1 at 75 mg/kg and 150 mg/kg BID×1 resulted in a modest PD effect at the protein and mRNA levels in the MT373 syngeneic model. The results show that treatment with Compound 1 led to a p53 conformation change from mutant to WT that functionally activates downstream signaling changes in the p53 pathway and related genes in the NF-κB pathway.

### Example 6: PKs and Brain Distribution of Compound 2 in Female CD-1 Mice Following a Single Oral Administration

The PK and brain distribution of Compound 2 in female CD-1 mice were determined following a single oral (PO) administration at 100 mg/kg.

Study Design: Twenty-one female CD-1 mice were treated with 100 mg/kg Compound 2 by oral gavage. The compound was formulated one day prior to dosing in 2% hydroxypropylcellulose (HPC) in water (w/v) at 10 mg/mL and administered at a concentration of 10 mL/kg. Three mice were sacrificed at 0.5, 1, 2, 4, 7, 10, and 24 hours post-dosing to collect blood and brain samples. The blood samples and brain homogenate were processed to determine concentrations of Compound 2 using liquid chromatography tandem mass spectrometry (LC-MS/MS). A bioanalytical assay for Compound 2 provided a lower limit of quantification (LLOQ) of 1 ng/mL for plasma and 7 ng/g for brain tissue with a linear range up to 3000 ng/mL for plasma and 21000 ng/g wet tissue for the brain. A non-compartmental PK model was employed to calculate PK parameters.

Formulation Analysis: Two aliquots of the dose solution, 20-50 μL, were sampled prior to dosing. The concentrations of Compound 2 in the dose solutions were measured by LC-MS/MS to determine the accuracy of the dose concentration. The formulation samples were quantified against a calibration curve consisting of six concentrations of Compound 2.

Animal Husbandry: The mice were group housed during acclimation and throughout the study under controlled temperature (20-26° C.), humidity (30-70%), and lights (12 h dark/light cycle). The animals were fed certified pellet diet. Water (reverse osmosis) was provided to the animals ad libitum. All mice were confirmed healthy prior to being assigned to the study. Each mouse was given a unique identification number, which was marked on the tail and written on the cage card as well. The animals were not fasted prior to compound administration, and food and water were present the entire time during the study.

Test Article Administration: The animals were weighed immediately prior to dosing. The body weight ranged from 21.4 g to 25.1 g. The dose volume was calculated individually for each mouse by multiplying the body weight and the nominal dose volume of 10 mL/kg. The compound was administered via oral gavage and the administered volume was verified by weighing the loaded and unloaded syringe before and after dosing. The weight difference (g) served as the confirmation of amount (mL) of dose solution dispensed. Cage side observations were performed before and after dosing as well as at each scheduled sample collection to look for signs of any adverse effects.

Sample Collection and Preparation: Blood sample: At each pre-defined time point, 3 mice were euthanized by CO₂ inhalation. After confirmation of death, blood samples were collected via cardiac puncture and placed in pre-chilled micro-tubes containing K₂EDTA as anti-coagulant. The collected blood samples were kept on ice until centrifugation. The blood samples were spun by centrifuge at 4° C., 3000 g for 15 min within half an hour of collection. Plasma was collected and placed in 96-well plates, quickly frozen on dry ice and stored at −70±10° C. until LC-MS/MS analysis. Brain: After blood collection, the brain was harvested, rinsed with cold distilled water, blotted dried, weighed, and quickly frozen on dry ice and stored under −70±10° C. until analysis. To prepare for bioanalytical assay, the brains were thawed at room temperature and homogenized using pre-chilled deionized water at the ratio of 1:6 (w/v: 1 g brain/6 mL water). The brain homogenates were then submitted for LC-MS/MS analysis.

Sample storage and processing: Study samples were stored in a freezer at a nominal temperature of −70° C. Brain tissues were homogenized with water at 1:6 weight to volume ratio using Omni bead rupture homogenizer. An aliquot of 20 μL study sample (plasma or tissue homogenate) was protein precipitated with 200 μL IS solution. Plasma samples were diluted as needed with blank mouse plasma. The IS used for Compound 2 was Labetalol. The IS solutions were made by dissolving the material in ACN at 100 ng/mL. The sample and IS solution mixture was stirred by vortex well and spun by centrifuge at 4000 rpm for 15 min, 4° C. An aliquot of 100 μL supernatant was transferred to sample plate and mixed with 100 μL water. The plate was shaken at 800 rpm for 10 min and then subject to LC/MS analysis.

Data analysis: The calibration curves of Compound 2 in CD-1 mouse plasma and CD-1 mouse brain tissue homogenate were constructed using 8 standards ranging from 1 to 3000 ng/mL. The regression analysis was performed by plotting the peak area ratio of test material over corresponding IS (y) against their concentration (x) in ng/mL, respectively. The fit equation for the calibration curve is linear with 1/x2 as weighting factor.

PK Analysis: The concentrations of Compound 2 in the plasma and brain samples were determined using a qualified LC-MS/MS method. The plasma and brain concentration-time data of Compound 2 from each animal were analyzed using Phoenix WinNonlin 6.3 to determine the PK properties of the compound in both matrices. A non-compartmental PK model and linear/log trapezoidal method were applied to PK calculations. The plasma or brain concentrations below the LLOQ before Tₘₐₓ were set to zero, and those after Tₘₐₓ were excluded from the PK calculation. The nominal dose level and nominal sample collection times were employed for the PK calculation. The values of plasma and brain concentrations as well as the PK parameters are reported in three significant figures. The average values of each dose group are presented as mean±SD.

Compound 2 at the administered dosage were well tolerated by all the animals. No overt adverse effects were observed throughout the study. The concentrations of Compound 2 in the dose suspensions were determined by LC-MS/MS to verify the dose accuracy. The measured concentration of Compound 2 in the formulation was 11.5 mg/mL (TABLE 21), within the acceptable range of ±20% of its nominal value 10 mg/mL.

TABLE 21

| | | Measured Conc. | | | | |
|---|---|---|---|---|---|---|
| | Dose | (mg/mL) | | Mean Conc. | Nominal Conc. | Accuracy^{a} |
| Test Article | (mg/kg) | Sample 1 | Sample 2 | (mg/mL) | (mg/mL) | (%) |
| Compound 2 | 100 | 11.7 | 11.4 | 11.5 | 10 | 115 |

^{a}Accuracy (%) = Mean Concentration (mg/mL)/Nominal Concentration (mg/mL) × 100.

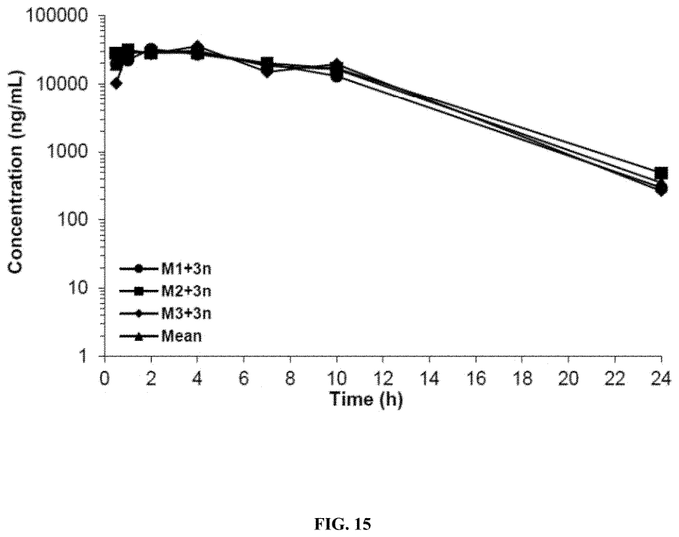

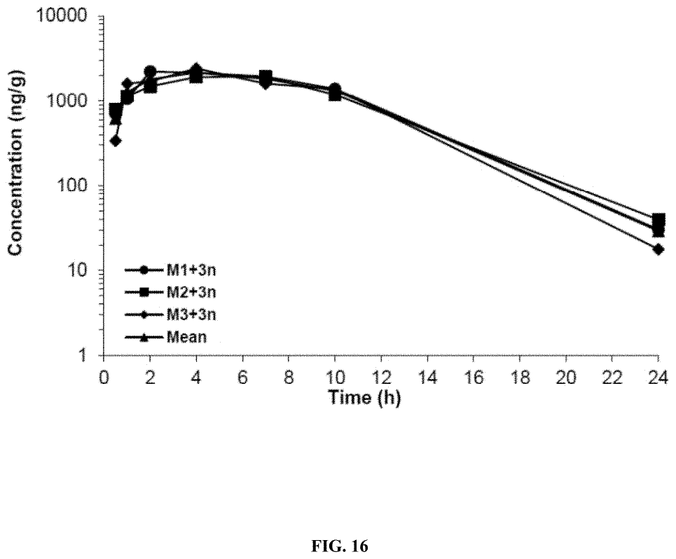

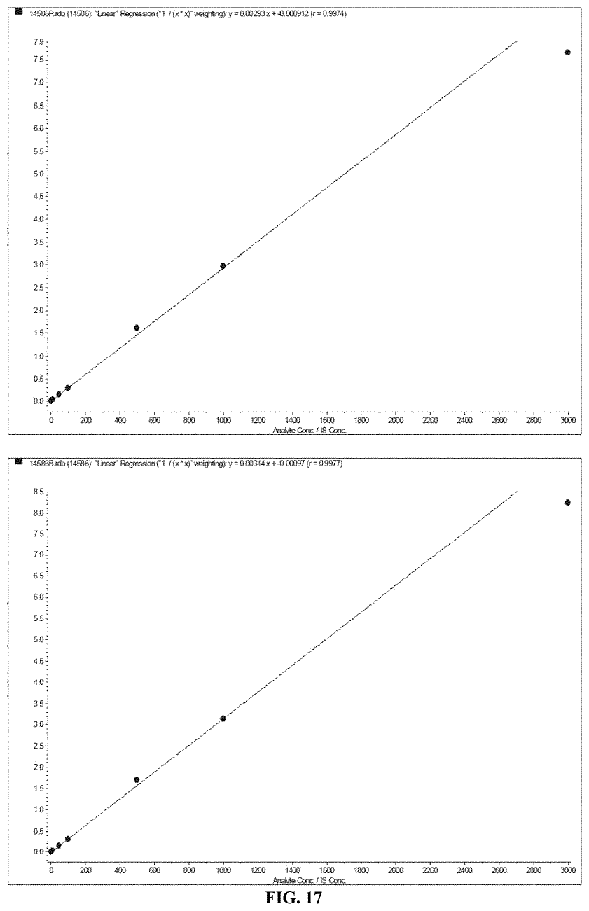

PK of Compound 2: The plasma and brain concentrations of Compound 2 are tabulated in TABLE 22 and TABLE 23. The plasma and brain PK parameters are summarized in TABLE 24, and the brain/plasma concentration ratios and the AUC ratios are shown in TABLE 25. The individual and mean plasma and brain concentration-time profiles of the test article were illustrated in FIG. 15 and FIG. 16. M #+3n: animal ID, 3 mice per time point identified by incremental numbers. FIG. 15 shows the individual and mean plasma concentration-time profile of Compound 2 in female CD-1 mice following a single PO administration at 100 mg/kg. FIG. 16 shows the individual and mean brain concentration-time profile of Compound 2 in female CD-1 mice following a single PO administration at 100 mg/kg. FIG. 17 shows calibration curves obtained for the test material in the sample run.

An oral administration of Compound 2 yielded a Cₘₐₓ of 30367 ng/mL in the plasma and of 2161 ng/g wet tissue in the brain, respectively. The Tₘₐₓ was 4.00 hours post dosing in both the plasma and brain. The T_{1/2} in the plasma and brain were 2.70 and 2.63 hours, respectively. The corresponding MRT₀₋ₗₐₛₜ were 6.42 and 7.01 hours. The AUC₀₋ₗₐₛₜ was 293660 ng·h/mL in the plasma and 21946 ng·h/g wet tissue in the brain, respectively. The brain (ng/g) to plasma (ng/mL) concentration ratio ranged between 0.0325-0.101 depending on sampling time. The brain/plasma AUC ratio for Compound 2 was 0.0747.

TABLE 22

| Sampling | Animal ID | | | | | | CV |
|---|---|---|---|---|---|---|---|
| Time (h) | n | M₁₊₃ₙ | M₂₊₃ₙ | M₃₊₃ₙ | Mean | SD | (%) |
| 0.500 | 0 | 19700 | 28100 | 10200 | 19333 | 8956 | 46.3 |
| 1.00 | 1 | 22400 | 30900 | 31200 | 28167 | 4996 | 17.7 |
| 2.00 | 2 | 32200 | 28300 | 27600 | 29367 | 3253 | 11.1 |
| 4.00 | 3 | 27000 | 28600 | 35500 | 30367 | 4517 | 14.9 |
| 7.00 | 4 | 19700 | 20100 | 14900 | 18233 | 2894 | 15.9 |
| 10.0 | 5 | 13000 | 16900 | 19400 | 16433 | 3225 | 19.6 |
| 24.0 | 6 | 301 | 493 | 269 | 354 | 121 | 34.2 |

M# + 3n: animal ID, 3 mice per time point identified by incremental numbers.

TABLE 23

| Sampling | Animal ID | | | | | | CV |
|---|---|---|---|---|---|---|---|
| Time (h) | n | M₁₊₃ₙ | M₂₊₃ₙ | M₃₊₃ₙ | Mean | SD | (%) |
| 0.500 | 0 | 707 | 798 | 338 | 614 | 244 | 39.6 |
| 1.00 | 1 | 1057 | 1141 | 1603 | 1267 | 294 | 23.2 |
| 2.00 | 2 | 2219 | 1484 | 1708 | 1804 | 361 | 20.0 |
| 4.00 | 3 | 2156 | 1911 | 2415 | 2161 | 252 | 11.7 |
| 7.00 | 4 | 1946 | 1932 | 1603 | 1827 | 194 | 10.6 |
| 10.0 | 5 | 1386 | 1183 | 1393 | 1321 | 119 | 9.03 |
| 24.0 | 6 | 30.3 | 39.6 | 17.9 | 29.3 | 10.9 | 37.3 |

M# + 3n: animal ID, 3 mice per time point identified by incremental numbers.

TABLE 24

| Parameter | Plasma | Brain |
|---|---|---|
| Rsq_adj | 0.983 | 0.996 |
| Cₘₐₓ (ng/mL) | 30367 | 2161 |
| Tₘₐₓ (h) | 4.00 | 4.00 |
| T_{1/2} (h) | 2.70 | 2.63 |
| Tₗₐₛₜ (h) | 24.0 | 24.0 |
| AUC₀₋ₗₐₛₜ (ng · h/mL) | 293660 | 21946 |
| AUC_{0-inf} (ng · h/mL) | 295041 | 22057 |
| MRT₀₋ₗₐₛₜ (h) | 6.42 | 7.01 |
| MRT_{0-inf} (h) | 6.52 | 7.11 |

TABLE 25

| Time (h) | Ratio* |
|---|---|
| 0.5 | 0.0325 |
| 1 | 0.0452 |
| 2 | 0.0611 |
| 4 | 0.0716 |
| 7 | 0.101 |
| 10 | 0.0828 |
| 24 | 0.0825 |
| AUC₀₋ₗₐₛₜ | 0.0747 |
| AUC_{0-inf} | 0.0748 |

*Values are mean of individual brain/plasma concentration ratios

Reference standard (RS) and Internal Standard (IS) are shown in TABLE 26.

TABLE 26

| Test Article | Purity (%) | Lot No. | Salt factor |
|---|---|---|---|
| Compound 2 | 96.9 | 5 | 1.00 |
| Labetalol (IS) | NA | L1011 | 1.00 |

The blank matrix was Matrix CD-1 mouse plasma (K₂EDTA). The concentrations of calibration standards and QC samples are shown in TABLE 27.

TABLE 27

| | Calibration Standard | Quality Control |
|---|---|---|
| | Samples Conc. | Samples Conc. |
| Matrix | (ng/mL) | (ng/mL) |
| CD-1 mouse plasma | 1, 2, 10, 50, 100, | 3, 40, 800, 2400 |
| (K2EDTA); | 500, 1000, 3000 | |
| CD-1 mouse brain | | |
| homogenate | | |

Results: Oral administration of Compound 2 yielded a peak concentration (Cₘₐₓ) of 30367 ng/mL in the plasma and 2161 ng/g wet tissue in the brain. The Cₘₐₓ was achieved at 4.00 hours (Tₘₐₓ) post-dosing in both the plasma and brain. The terminal elimination half-lives (T_{1/2}) in the plasma and brain were 2.70 and 2.63 hours, respectively. The corresponding mean residence times (MRT₀₋ₗₐₛₜ) in the plasma and brain were 6.42 and 7.01 hours, respectively. The areas under the concentration-time curve (AUC₀₋ₗₐₛₜ) was 293660 ng·h/mL in the plasma and 21946 ng·h/g wet tissue in the brain. The test articles at the administered dosage were well tolerated by the animals. No overt adverse effects were evident throughout the study. A summary of the key PK parameters based on individual concentration-time data as well as the brain/plasma concentration ratios are shown in TABLE 28.

TABLE 28

| PK parameters | | | Brain/plasma concentration ratio | |
|---|---|---|---|---|
| | Plasma | Brain | Time (h) | Ratio |
| Cₘₐₓ (ng/mL) | 30367 | 2161 | 0.5 | 0.0325 |
| Tₘₐₓ (h) | 4.00 | 4.00 | 1 | 0.0452 |
| T_{1/2} (h) | 2.70 | 2.63 | 2 | 0.0611 |
| AUC₀₋ₗₐₛₜ (ng · h/mL) | 293660 | 21946 | 4 | 0.0716 |
| AUC_{0-inf} (ng · h/mL) | 295041 | 22057 | 7 | 0.101 |
| MRT₀₋ₗₐₛₜ (h) | 6.42 | 7.01 | 10 | 0.0828 |
| MRT_{0-inf} (h) | 6.52 | 7.11 | 24 | 0.0825 |

### Example 7: Efficacy and Tolerability of Compound 2 in a Mouse Xenograft Model of Gastric Cancer (NUGC3) when Administered 150 mg/kg (2Q7D×4) and 300 mg/kg (2Q7D×5)

The efficacy of Compound 2 was tested at two dose levels in a mouse xenograft model of gastric cancer (NUGC3). Female nude mice were implanted with NUGC3 cells and tumors were grown to ˜240 mm³ prior to being randomized into one of three study groups. TABLE 29 shows efficacy study groups and dosing regimen. Group 1 mice were dosed PO with vehicle control (0.2% HPC, 0.5% Tween 80) once daily for 21 days (QD×21). Group 11 was dosed PO with Compound 2 twice daily (BID) once per week for five doses (2Q7D×5) at 300 mg/kg while group 12 received Compound 2 at 150 mg/kg 2Q7D×4. All mice across the study had tumors and plasma harvested for PD analysis 24 h post final dose.

TABLE 29

| | | | | Dosing | | Dose |
|---|---|---|---|---|---|---|
| | | | | Frequency & | Dose | Volume |
| Group | Treatment | N | Route | Duration | (mg/kg) | (mL/kg) |
| 1 | Vehicle Control | 10 | PO | QDx1 | N/A | 10 |
| 11 | Compound 2 | 10 | PO | 2Q7Dx5 | 300 | 10 |
| 12 | Compound 2 | 10 | PO | 2Q7Dx4 | 150 | 10 |

Animals: Female Balb/c nude mice (300 total) were acclimatized for 1 week and were 8-10 weeks old at initiation of study. Animals were group housed (N=5) in ventilated cages. Fluorescent lighting was provided on a 12-hour cycle (6:30 am-6:30 pm). Temperature and humidity were monitored and recorded daily and maintained at 68-72° F. (20-22.2° C.) and 30-70% humidity. 18% soy irradiated rodent feed and autoclaved acidified water (pH 2.5-3) was provided ad libitum.

Tumor Cell Culture: NUGC3 cells were cultured in RPMI 1640 medium with 10% fetal bovine serum. The cells were washed with PBS and counted at a total of 6.54×10⁸ cells with 93.7% viability. Cells were centrifuged and resuspended in 50% PBS:50% Matrigel Matrix at a concentration of 1×10⁸ viable cells/100 μL.

Implantation of Mice: Cells were prepared for injections by drawing the cell suspension into a 1-mL tuberculin syringe fitted with a 25G ⅝″ needle. Individual mice were manually restrained, the site of injection (right flank) was disinfected with a 70% ethanol swab, and 100 μL of cell suspension was injected subcutaneously.

Randomization and Study Setup: Implanted animals were monitored for palpable tumors. Twelve days post implant the animals with palpable tumors had their tumor sizes determined via digital caliper. Mice were selected and randomized into three treatment groups according to tumor size. Treatment began on the thirteenth day post-implant to facilitate BID dosing. Average tumor volume (mm³) and body weight (g) are described in TABLE 30.

TABLE 30

| | | Tumor Volume (mm³) | | | | Body Weight (g) | | | |
|---|---|---|---|---|---|---|---|---|---|
| Group | N | Mean | St Dev | Min | Max | Mean | St Dev | Min | Max |
| 1 - Vehicle Control | 10 | 244.21 | ±39.77 | 166.28 | 295.25 | 22.83 | ±2.70 | 19.90 | 29.20 |
| 11 - Compound 2 at | 10 | 246.53 | ±36.52 | 191.88 | 297.60 | 22.64 | ±1.71 | 21.00 | 26.10 |
| 300 mg/kg 2Q7Dx5 | | | | | | | | | |
| 12 - Compound 2 at | 10 | 246.69 | ±31.86 | 194.21 | 292.96 | 22.63 | ±1.42 | 19.90 | 24.10 |
| 150 mg/kg 2Q7Dx4 | | | | | | | | | |

Measurements and Calculation of Tumor Volume; and tumor lysate preparation were performed as described in EXAMPLE 5.

Mutant, WT, and total p53 ELISA: 96-well ELISA plates were coated with either WT p53 (150 ng/well; PAb1620), mutant p53 (100 ng/well; PAb240), or total p53 (31.3 ng/well; PAb1801) antibodies and incubated overnight at 4° C. Plates were washed with wash buffer (PBS±0.05% Tween 20) and treated with blocking buffer (PBS±1% BSA±0.05% Tween 20) for 1 h and then washed. Tumor lysates were diluted in blocking buffer such that the desired protein amount is added to the plate in a 100 μL volume (WT p53 50 μg; mutant p53 12.5 μg; Total p53 5 μg). Lysates were incubated overnight at 4° C. with shaking. Plates were again washed and treated with detection antibody diluted in blocking buffer (0.025 mg/mL; biotinylated p53) for 1 h, plates were washed and finally incubated in streptavidin-HRP (1:10000) diluted in blocking buffer for 30 minutes. Plates were washed, and the reaction developed using TMB for approximately 5 minutes and the reaction quenched with 0.16 M sulfuric acid (H₂SO₄). Plates were read on a plate reader at 450 nm. A background measurement was subtracted from the treated samples signal and they were normalized to their respective vehicle controls.

p21, MDM2, and MIC1 ELISA: Polystyrene 96-well plates were coated with the respective capture antibody and incubated overnight at 4° C. Plates were then washed in wash buffer and blocked for 1 h. Tumor lysates (12.5 μg p21, 75 μg MDM2, or 25 μg MIC-1) or plasma (MIC-1) were diluted to the appropriate concentration and added in a volume of 100 μL. Additionally, a 7-point standard curve was also added to the plates. Plates were incubated at either 2 h at room temperature (p21, MIC-1 plasma) or 4° C. overnight (MDM2, MIC-1 protein), shaking. The plates were then washed and incubated in detection antibody for 2 h. Plates were washed and incubated in streptavidin-HRP for 30 min. Finally, plates were washed, and the reaction developed using TMB substrate for 10 min. The reaction was quenched with stop solution (0.16 M H₂SO₄) and the plates read at 450 and 570 nm. Protein levels for both analytes were quantified using the provided standard curve.

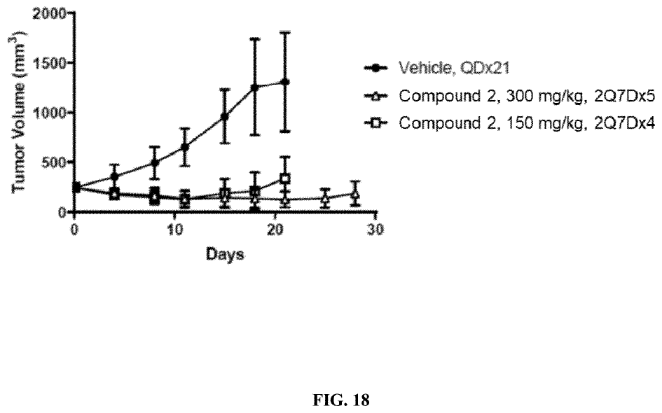

Efficacy: NUGC3 human gastric tumors grown in the female nude mice grew from an average of 244 mm³ to 1308 mm³ in 21 days. Mice treated with Compound 2 at 150 mg/kg and 300 mg/kg Q7D×4-5 resulted in 92% TGI and 48% regression, respectively, by day 21 of the study (FIG. 18). TABLE 31 shows average percent tumor growth inhibition in %. TABLE 32 shows average percent tumor regression inhibition in %.

TABLE 31

| Day of Study | 4 | 8 | 11 | 15 | 18 | 21 |
|---|---|---|---|---|---|---|
| Group 1 - | 0 | 0 | 0 | 0 | 0 | 0 |
| Vehicle Control | | | | | | |
| Group 11 - | >100 | >100 | >100 | >100 | >100 | >100 |
| Compound 2 | | | | | | |
| 300 mg/kg | | | | | | |
| 2Q7Dx5 | | | | | | |
| Group 12 - | >100 | >100 | >100 | >100 | >100 | 91.5 |
| Compound 2 | | | | | | |
| 150 mg/kg | | | | | | |
| 2Q7Dx4 | | | | | | |

TABLE 32

| Day of Study | 4 | 8 | 11 | 15 | 18 | 21 |
|---|---|---|---|---|---|---|
| Group 1 - | 0 | 0 | 0 | 0 | 0 | 0 |
| Vehicle Control | | | | | | |
| Group 11 - | 23.4 | 32.1 | 45.4 | 41.7 | 44.1 | 48.2 |
| Compound 2 | | | | | | |
| 300 mg/kg | | | | | | |
| 2Q7Dx5 | | | | | | |
| Group 12 - | 29.9 | 39.7 | 47.4 | 23.6 | N/A | N/A |
| Compound 2 | | | | | | |
| 150 mg/kg | | | | | | |
| 2Q7Dx4 | | | | | | |

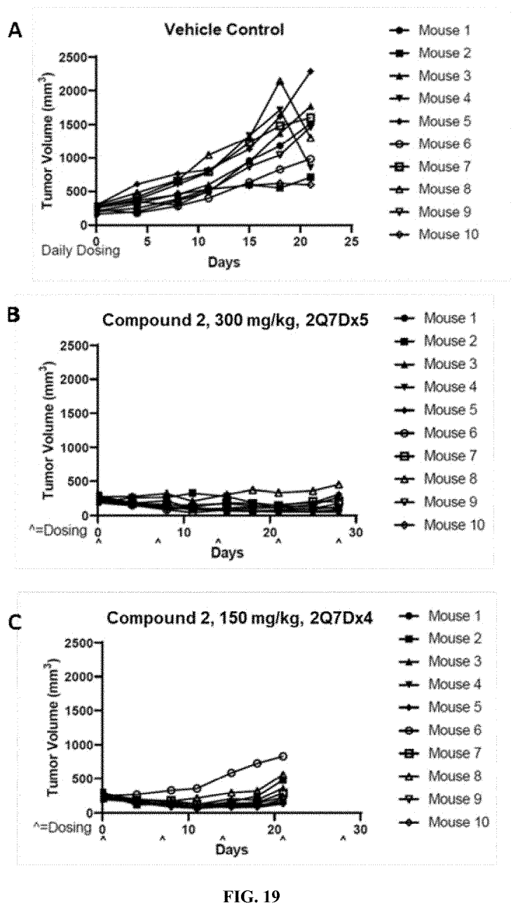

FIG. 19 shows individual tumor volumes across a study of 10 mice treated with control, Compound 2 300 mg/kg 2Q7D×5, or 150 mg/kg 2Q7D×4. NUGC3 tumors treated with vehicle control (0.2% HPC, 0.5% Tween 80) displayed consistent growth across 21 days of study with the exception of mouse 4 and 8 where the tumor collapsed due to necrosis at the day 21 measurement. Nine out of ten mice administered Compound 2 at 300 mg/kg (2Q7D×5) showed tumor regression out to day 28 of study, while mouse #8 experienced stasis. Mice receiving Compound 2 at 150 mg/kg (2Q7D×4) demonstrated consistent regression across most animals through 18 days except for mouse 6 where tumor control was measured and then lost after day 11.

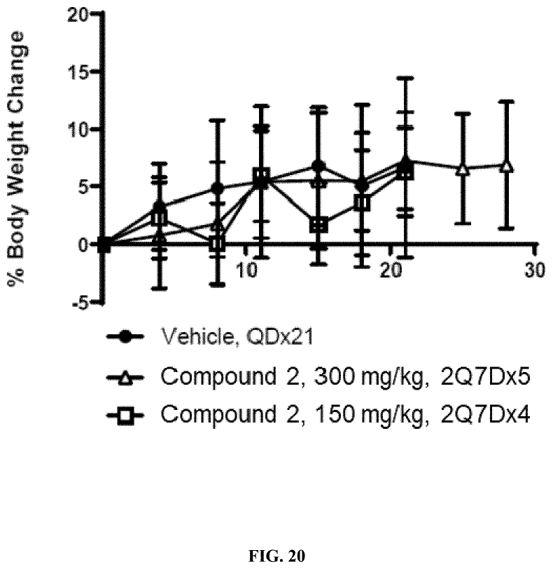

Body Weights: FIG. 20 shows average percent change in body weight across a study (%, average±SD) of mice treated with vehicle QD×21, Compound 2 300 mg/kg 2Q7D×5, or 150 mg/kg 2Q7D×4. Average mouse body weights were well maintained over the course of the study. TABLE 33 shows average percent change in body weight across study (n=8 unless otherwise noted). Average mouse body weights were well maintained over the course of the study. Mice dosed with vehicle control remained on average around 23.7 g throughout the study with the percentage change varying between 3.28% and 6.79%. Dosing with Compound 2 did not result in any body weight loss on average at any point along the study. By day 21, weight gain of 7.26% and 6.31% was observed in the 300 mg/kg and the 150 mg/kg groups, respectively.

TABLE 33

| | | Group 11 | Group 12 |
|---|---|---|---|
| | Group 1 | Compound 2 | Compound 2 |
| Day of | Vehicle | 300 mg/kg | 150 mg/kg |
| Study | Control | 2Q7Dx5 | 2Q7Dx4 |
| 4 | 3.28 ± 3.74 | 0.79 ± 4.56 | 2.30 ± 3.52 |
| 8 | 4.83 ± 5.94 | 1.84 ± 5.30 | 0.10 ± 3.49 |
| 11 | 5.40 ± 6.58 | 5.40 ± 4.85 | 5.97 ± 3.91 |
| 15 | 6.79 ± 5.07 | 5.53 ± 5.88 | 1.77 ± 3.51 |
| 18 | 5.06 ± 7.05 | 5.47 ± 4.25 | 3.63 ± 4.60 |
| 21 | 6.65 ± 7.81 | 7.26 ± 4.20 | 6.31 ± 3.84 |
| 25 | — | 6.58 ± 4.76 | — |
| 28 | — | 6.88 ± 5.46 | — |

Compound 2 was well tolerated throughout the study with no clinical observations to report. TABLE 34 summarizes clinical observations from the study.

TABLE 34

| | Mouse | Date of Death | |
|---|---|---|---|
| Group | Number | (Study Day) | Clinical Observations |
| Group 1 | 1 | 21 | None; euthanized for end of study 8 h post-dose |
| Vehicle | 2 | 21 | None; euthanized for end of study 8 h post-dose |
| Control | 3 | 21 | None; euthanized for end of study 8 h post-dose |
| QDx21 | 4 | 21 | Tumor noted grossly necrotic on day 21; euthanized for |
| | | | end of study 8 h post-dose |
| | 5 | 21 | None; euthanized for end of study 8 h post-dose |
| | 6 | 21 | None; euthanized for end of study 24 h post-dose |
| | 7 | 21 | None; euthanized for end of study 24 h post-dose |
| | 8 | 21 | Tumor noted grossly necrotic on day 21; euthanized for |
| | | | end of study 24 h post-dose |
| | 9 | 21 | None; euthanized for end of study 24 h post-dose |
| | 10 | 21 | None; euthanized for end of study 24 h post-dose |
| Group 11 | 1 | 29 | None; euthanized for end of study 24 h post-dose |
| Compound 2 | 2 | 29 | None; euthanized for end of study 24 h post-dose |
| 300 mg/kg | 3 | 29 | None; euthanized for end of study 24 h post-dose |
| 2Q7Dx5 | 4 | 29 | None; euthanized for end of study 24 h post-dose |
| | 5 | 29 | None; euthanized for end of study 24 h post-dose |
| | 6 | 29 | None; euthanized for end of study 24 h post-dose |
| | 7 | 29 | None; euthanized for end of study 24 h post-dose |
| | 8 | 29 | Tumor noted as fluid filled on Day 11; euthanized for |
| | | | end of study 24 h post-dose |
| | 9 | 29 | None; euthanized for end of study 24 h post-dose |
| | 10 | 29 | None; euthanized for end of study 24 h post-dose |
| Group 12 | 1 | 22 | None; euthanized for end of study 24 h post-dose |
| Compound 2 | 2 | 22 | None; euthanized for end of study 24 h post-dose |
| 150 mg/kg | 3 | 22 | None; euthanized for end of study 24 h post-dose |
| 2Q7Dx4 | 4 | 22 | None; euthanized for end of study 24 h post-dose |
| | 5 | 22 | None; euthanized for end of study 24 h post-dose |
| | 6 | 22 | None; euthanized for end of study 24 h post-dose |
| | 7 | 22 | None; euthanized for end of study 24 h post-dose |
| | 8 | 22 | None; euthanized for end of study 24 h post-dose |
| | 9 | 22 | None; euthanized for end of study 24 h post-dose |
| | 10 | 22 | None; euthanized for end of study 24 h post-dose |

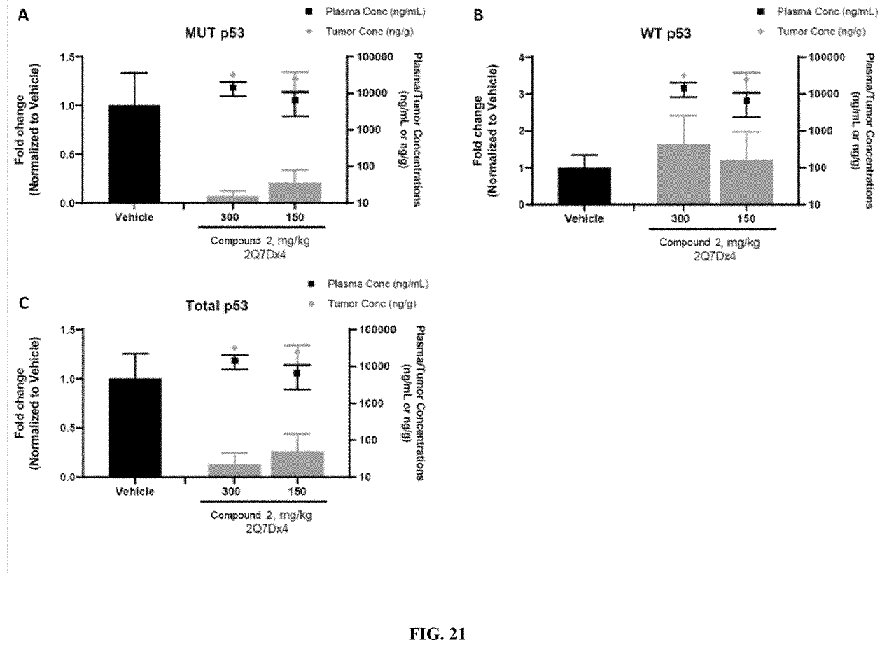

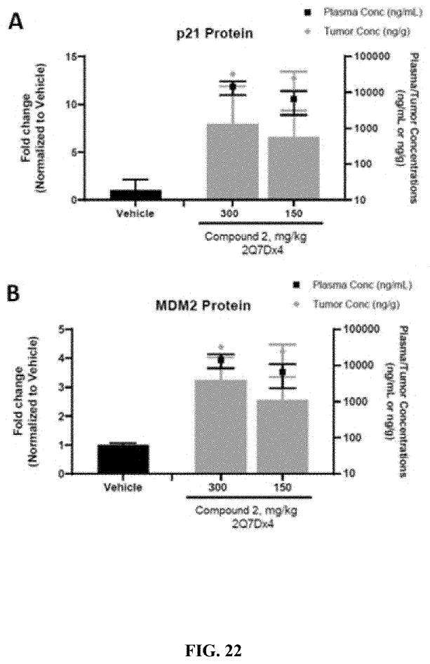

End of Efficacy PK/PD Results: All mice had tumors and plasma harvested for PD/PK analysis 24 h post the final dose. TABLE 35 summarizes results of the PD/PK analysis. Doses of 150 mg/kg and 300 mg/kg 2Q7D resulted in dose-proportional changes in plasma concentrations of 14320 ng/mL and 6537 ng/mL 24 h post-dose, respectively. Modulation of targets were dose responsive. Tumors from mice dosed with Compound 2 at 150 mg/kg resulted in a 1.2-fold increase in WT conformation p53, a 79.2% decrease in mutant p53 and a 73.6% decrease in total levels of p53. Tumors from mice that received Compound 2 at 300 mg/kg showed a 1.6-fold increase in WT conformation p53, a 92.7% decrease in mutant p53 and an 87.1% decrease in total levels of p53. FIG. 21 PANEL A-PANEL C show conversion of mutant p53 to wild-type p53 conformation in mice treated with vehicle, Compound 2 300 mg/kg 2Q7D×4, or 150 mg/kg 2Q7D×4. Measurement of p53 target proteins downstream of WT conformation p53 revealed a 6.59-fold increase in p21 and a 2.56-fold increase in MDM2 in tumors of mice dosed with 150 mg/kg 2Q7D×4 at 24 h. Tumors from mice dosed at 300 mg/kg 2Q7D×5 resulted in a 7.96-fold increase in p21 protein and a 3.26-fold increase in MDM2 24 h post dose. FIG. 22 PANEL A and PANEL B show that conversion of mutant to WT conformation p53 results in downstream increases in p53 target proteins: p21 and MDM2 in mice treated with Compound 2 300 mg/kg 2Q7D×4 or 150 mg/kg 2Q7D×4.

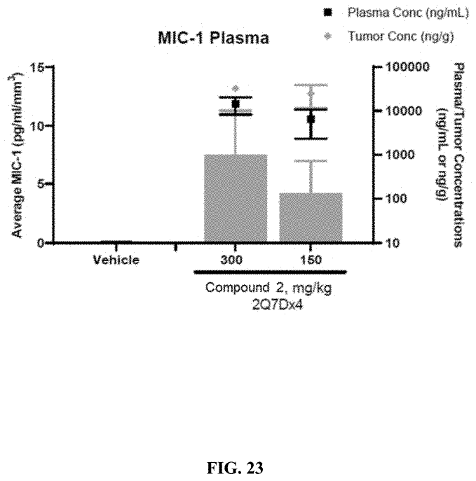

Dose responsive increases in MIC-1 cytokine levels can be measured in the plasma of the mice and normalized to individual tumor volume as MIC-1 cytokine is not expressed in vehicle control treated tumors. Mice treated with 150 mg/kg Compound 2 2Q7D×4 plasma MIC-1 levels measured 4.21 μg/mL/mm³ 24 h post-doing while those receiving 300 mg/kg 2Q7D×5 measured 7.52 μg/mL/mm³ 24 h post-dosing. FIG. 23 shows average MIC-1 plasma levels (pg/mL/mm³) and plasma (ng/mL) and tumor (ng/g) concentrations of MIC-1 plasma in mice treated with vehicle, 150 mg/kg Compound 2, and 300 mg/kg Compound 2.

TABLE 35

| | | | Fold Change over Vehicle Control or Percent Reduction | | | | | |
|---|---|---|---|---|---|---|---|---|
| | | | Relative to Vehicle Control (%) | | | | | |
| Group - | Plasma | Tumor | Mutant | WT | Total | | | |
| Compound 2 | Conc. | Conc. | p53 | p53 | p53 | p21 | MDM2 | MIC-1 |
| (mg/kg) | (ng/mL) | (ng/g) | Protein | Protein | Protein | Protein | Protein | Protein |
| Group 11 - | 14320 | 32270 | 92.7% | 1.63 | 87.1% | 7.96 | 3.26 | 7.52* |
| 300 mg/kg | | | | | | | | |
| 2Q7Dx5 | | | | | | | | |
| Group 12 - | 6537 | 24663 | 79.2% | 1.21 | 73.6% | 6.59 | 2.56 | 4.21* |
| 150 mg/kg | | | | | | | | |
| 2Q7Dx4 | | | | | | | | |

*= Absolute induction in pg/mL/mm³.

The anti-tumor effect of Compound 2 was tested in a mouse xenograft model of gastric cancer (NUGC3) at two dose levels. Compound 2 was administered PO at 300 mg/kg 2Q7D×5 or 150 mg/kg 2Q7D×4 resulting in 48.2% regression and 91.5% TGI, respectively, at day 21 of study. Animals receiving Compound 2 at 300 mg/kg were allowed to stay on study for an additional week, but the regression did not increase. During this study Compound 2 was well tolerated throughout the dosing period with mice showing overall body weight gains and no clinical observations.

Tumor and plasma for were collected for PK/PD analysis 24 h post the final dose. Plasma concentrations were in the expected range for the dose levels and resulted in dose proportional plasma exposure between the two dose levels. Tumors harvested from Compound 2 treated mice showed increases in WT conformation p53 protein (1.2- to 1.6-fold) and dose dependent reductions in both mutant p53 (79.2-92.7%) and total p53 (73.6-87.1%) when compared to vehicle control. Analysis of the downstream p53 transcriptional targets revealed dose responsive increases in both p21 protein (6.5- to 7.9-fold) and MDM2 (2.5- to 3.2-fold) correlating with dose proportional plasma exposure. Absolute MIC-1 levels (4.2-7.5 μg/mL/mm³) were measured in the plasma and likewise demonstrated an increase with increasing dose. Overall, oral once-weekly administration of Compound 2 was well tolerated and resulted in a dose responsive anti-tumor effect of strong tumor growth delay to robust tumor regression in a dose proportional manner. These strong anti-tumor effects correlated with a dose responsive PD effect.

### Example 8: Efficacy and Tolerability of Compound 2 in a Mouse Xenograft Model of Gastric Cancer (NUGC3) when Administered 100 mg/kg (QD×44) and 300 mg/kg (Q3D×11)

The efficacy of Compound 2 was tested at two dose levels in a mouse xenograft model of gastric cancer (NUGC3). Nude mice were implanted with NUGC3 cells and tumors were grown to ˜225 mm³ prior to being randomized into one of three study groups. Mice were dosed orally (PO) with either vehicle control (0.2% HPC, 0.5% Tween 80) twice daily for 3 weeks (BID×21), matching the most rigorous regimen of other compounds not included in this report, or with Compound 2 at 100 mg/kg daily for 6 weeks (QD×44) and 300 mg/kg twice weekly for 5 weeks (Q3D×11).

Study design: TABLE 36 shows efficacy study groups and dosing regimen. Group 1 mice were dosed PO with vehicle control (0.2% HPC, 0.5% Tween 80) twice daily for 21 days (BID×21). Group 8 was dosed PO with Compound 2 daily for 44 days (QD×44) at 100 mg/kg while group 10 received Compound 2 PO twice weekly (Q3D×11) at 300 mg/kg. At the conclusion of the study mice with sufficiently large tumors in the Compound 2 300 mg/kg Q3D×11 group had tumor and plasma harvested for PD analysis 8 h post final dose, while animals in the 100 mg/kg group had only plasma collected.

TABLE 36

| | | | | Dosing | | Dose |
|---|---|---|---|---|---|---|
| | | | | Frequency & | Dose | Volume |
| Group | Treatment | N | Route | Duration | (mg/kg) | (mL/kg) |
| 1 | Vehicle Control | 9 | PO | BIDx21 | N/A | 10 |
| 8 | Compound 2 | 10 | PO | QDx44 | 100 | 10 |
| 10 | Compound 2 | 10 | PO | Q3Dx11 | 300 | 10 |

Animals, Tumor cell culture, implantation of mice, randomization and study set up procedures were used as described in EXAMPLE 7. Average tumor volume (mm³) and body weight (g) is described m TABLE 37.

TABLE 37

| | | Tumor Volume (mm³) | | | | Body Weight (g) | | | |
|---|---|---|---|---|---|---|---|---|---|
| Group | N | Mean | St Dev | Min | Max | Mean | St Dev | Min | Max |
| 1 - Vehicle Control | 8 | 225.32 | ±39.93 | 141.89 | 273.76 | 23.45 | ±1.30 | 21.20 | 24.80 |
| 8 - Compound 2 at | 8 | 229.31 | ±32.10 | 172.82 | 268.14 | 23.69 | ±1.08 | 22.20 | 25.10 |
| 100 mg/kg QDx44 | | | | | | | | | |
| 10 - Compound 2 at | 8 | 227.50 | ±32.47 | 176.63 | 265.87 | 23.59 | ±1.16 | 21.80 | 25.00 |
| 300 mg/kg Q3Dx11 | | | | | | | | | |

Measurements and Calculation of Tumor Volume; tumor lysate preparation were performed as described in EXAMPLE 5. The MDM2 ELISA assay was performed as descried in EXAMPLE 5.

Mutant, WT, and total p53 ELISA: p53 (5 μg/mL), WT p53 (10 μg/mL), total p53 (5 μg/mL), and p21 Waf1/Cip1 (0.5 μg/mL) antibodies were coupled with U-Plex linkers by combining optimized concentrations for each antibody with the assigned linker, agitated by vortex, and incubated for 30 minutes at RT before adding a Stop Solution and incubating for another 30 minutes. The coupled antibody-linkers were combined into the same tube and the total volume adjusted with Stop Solution to 12 mL final volume. 96-well MSD U-Plex plates were coated with 50 μL/well of combined antibody-linker solution and incubated overnight at 4° C. on a shaker. Plates were washed 3× with wash buffer (1×TBS±0.1% Tween 20) and blocked with 1× blocking buffer (1×TBS±0.1% Tween 20±3% BSA). Tumor lysates were diluted in 1× lysis buffer to 0.4 μg/μL, blocking buffer was aspirated from the MSD plate, and 50 μL of tumor lysate was added to each well. The plate was sealed and incubated overnight at 4° C. on a shaker. Plates were washed 3× and treated with 50 μL/well detection antibody diluted in antibody diluent (1×TBS±0.1% Tween 20±1% BSA) (0.05 μg/mL; p53 7F5 Rabbit mAb, 0.05 μg/mL; p21 12D1 Rabbit mAb) for 1 h at RT. The plate was washed 3×, and the secondary antibody (Goat anti-Rabbit SULFO-TAG at 1 μg/mL) was added at 50 μL/well and incubated for 1 h at RT on a shaker. The plate was finally washed 3×, 2×MSD Read Buffer was added at 150 μL/well and the plate was read immediately on the MESO QuickPlex SQ 120.

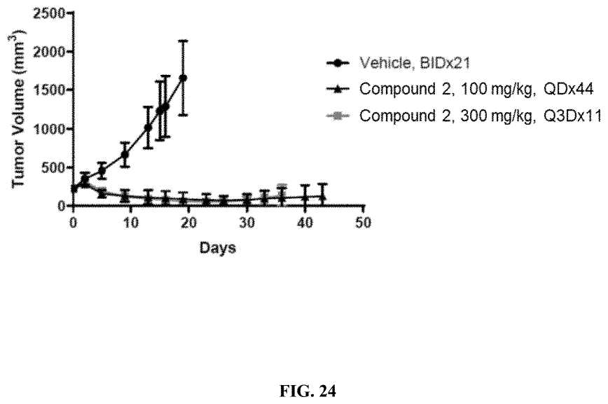

Efficacy: NUGC3 human gastric tumors grown in the female nude mice grew from an average of 225 mm³ to 1658 mm³ in 19 days. Tumors on mice administered Compound 2 at 100 mg/kg QD×44 resulted in 60% regression by day 19 and remained on study for 44 days reaching a maximum 69% regression on day 26. Tumors on mice receiving Compound 2 at 300 mg/kg Q3D×11 resulted in 74% regression by day 19 and a maximum 75% regression on day 23. TABLE 38 shows average percent tumor growth inhibition in %. TABLE 39 shows average percent tumor regression inhibition in %. FIG. 24 shows changes in tumor volume (mm³) in NUGC3 human gastric tumors grown in female nude mice upon treatment with vehicle (BID×21), 100 mg/kg Compound 2 (QD×44), and 300 mg/kg Compound 2 (Q3D×11).

TABLE 38

| Day of Study | 4 | 8 | 11 | 15 | 18 | 21 |
|---|---|---|---|---|---|---|
| Group 1 - | 0 | 0 | 0 | 0 | 0 | 0 |
| Vehicle Control | | | | | | |
| Group 8 - | >100 | >100 | >100 | >100 | >100 | >100 |
| Compound 2 | | | | | | |
| 100 mg/kg QDx44 | | | | | | |
| Group 10 - | >100 | >100 | >100 | >100 | >100 | 91.5 |
| Compound 2 | | | | | | |
| 300 mg/kg | | | | | | |
| Q3Dx11 | | | | | | |

TABLE 39

| Day of Study | 2 | 5 | 9 | 13 | 16 | 19 | 23 | 26 | 30 | 33 | 36 | 40 | 43 |
|---|---|---|---|---|---|---|---|---|---|---|---|---|---|
| Group 1 - Vehicle Control | 0 | 0 | 0 | 0 | 0 | 0 | N/A | N/A | N/A | N/A | N/A | N/A | N/A |
| Group 8 - Compound 2 | 0 | 29.9 | 42.7 | 51.1 | 56.3 | 60.8 | 66.6 | 69.6 | 66.3 | 58.0 | 54.0 | 47.5 | 44.3 |
| 100 mg/kgQDx44 | | | | | | | | | | | | | |
| Group 10 - Compound 2 | 0 | 14.2 | 45.8 | 59.8 | 65.7 | 74.2 | 75.6 | 75.2 | 67.6 | 53.3 | 39.7 | N/A | N/A |
| 300 mg/kgQ3Dx11 | | | | | | | | | | | | | |

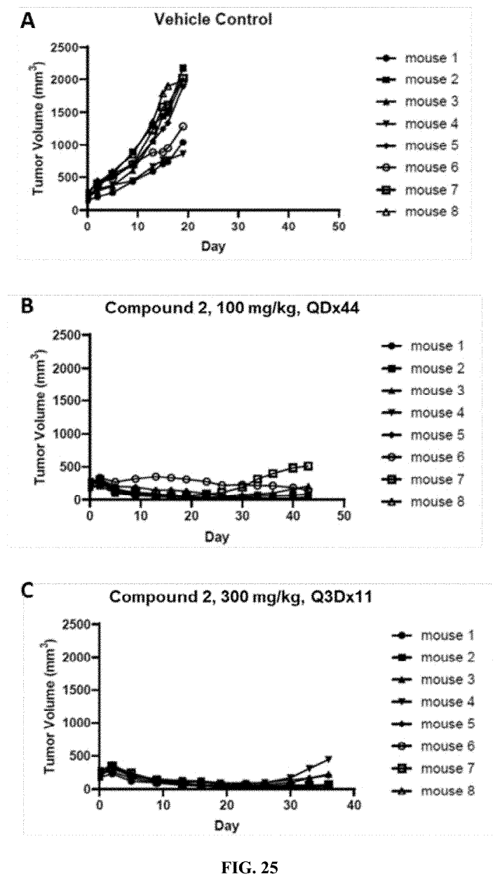

FIG. 25 PANEL A-PANEL C show individual tumor volumes across the study in mice treated with vehicle, Compound 2 100 mg/kg QD×44, or 300 mg/kg Q3D×11. NUGC3 tumors treated with vehicle control (0.2% HPC, 0.5% Tween 80) displayed consistent growth across 19 days of study. All mice (n=8) administered Compound 2 at 100 mg/kg (QD×44) showed tumor regression out to day 19 of study with the exception of mouse 6 which did not reach regression until day 26. Mice receiving Compound 2 at 300 mg/kg (Q3D×11) demonstrated consistent regression across all animals through 26 days.

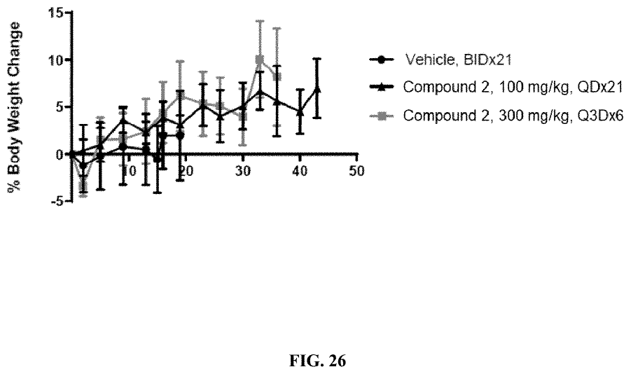

Body Weights: FIG. 26 shows average percent change in body weight across study (%, average±SD) in mice treated with vehicle BID×21, Compound 2 100 mg/kg QD×21, or 300 mg/kg Q3D×6. Average mouse body weights were well maintained over the course of the study. Mice dosed with vehicle control remained on average around 23.5 g throughout the study with the percentage change varying between −1.33% and 1.87%. Dosing with Compound 2 at 100 mg/kg QD×44 did not result in any body weight loss on average throughout the study, though administration of 300 mg/kg Q3D×11 did result in −3.39% weight loss on day 2 that recovered to 1.48% on day 5. By day 36, weight gain of 5.60% and 8.15% was observed in the 100 mg/kg and the 300 mg/kg groups, respectively. TABLE 40 shows average percent change in body weight across study (n=8 unless otherwise noted).

TABLE 40

| | | Group 8 | Group 10 |
|---|---|---|---|
| | Group 1 | Compound 2 | Compound 2 |
| Day of | Vehicle | 100 mg/kg | 300 mg/kg |
| Study | Control | QDx44 | Q3Dx11 |
| 2 | −1.23 ± 2.80 | 0.40 ± 2.70 | −3.39 ± 1.08 |
| 5 | −0.19 ± 3.57 | 0.98 ± 1.79 | 1.55 ± 2.36 |
| 9 | 0.81 ± 4.06 | 3.64 ± 1.38 | 1.56 ± 2.80 |
| 13 | 0.51 ± 3.77 | 2.28 ± 1.19 | 2.40 ± 3.44 |
| 15 | −0.57 ± 3.55 | * | * |
| 16 | 1.95 ± 3.58 | 3.87 ± 1.68 | 4.39 ± 3.23 |
| 19 | 1.95 ± 4.74 | 3.12 ± 1.00 | 6.16 ± 3.70 |
| 23 | — | 5.18 ± 2.20 | 5.30 ± 3.39 |
| 26 | — | 4.02 ± 2.76 | 5.08 ± 3.00 |
| 30 | — | 5.08 ± 2.47 | 3.94 ± 3.01 |
| 33 | — | 6.69 ± 1.98 | 10.06 ± 4.08 |
| 36 | — | 5.60 ± 3.73 | 8.15 ± 5.15 |
| 40 | — | 4.50 ± 2.37 | — |
| 43 | — | 6.98 ± 3.14 | — |

* = Measurement not recorded.

Clinical Observations: Compound 2 was well tolerated throughout the study with no clinical observations to report. TABLE 41 summarizes clinical observations from the study.

TABLE 41

| | Mouse | Date of Death | |
|---|---|---|---|
| Group | Number | (Study Day) | Clinical Observations |
| Group 1 | 1 | 20 | None; euthanized for end of study 4 h post-dose |
| Vehicle | 2 | 20 | None; euthanized for end of study 4 h post-dose |
| Control | 3 | 20 | None; euthanized for end of study 4 h post-dose |
| 2QDx21 | 4 | 20 | None; euthanized for end of study 4 h post-dose |
| | 5 | 20 | None; euthanized for end of study 4 h post-dose |
| | 6 | 20 | None; euthanized for end of study 4 h post-dose |
| | 7 | 20 | None; euthanized for end of study 4 h post-dose |
| | 8 | 20 | None; euthanized for end of study 4 h post-dose |
| Group 8 | 1 | 43 | None; euthanized for end of study 8 h plasma only |
| Compound 2 | | | collection post-dose |
| 100 mg/kg | 2 | 44 | None; euthanized for end of study |
| QDx44 | 3 | 44 | None; euthanized for end of study |
| | 4 | 43 | None; euthanized for end of study 8 h plasma only |
| | | | collection post-dose |
| | 5 | 43 | None; euthanized for end of study 8 h plasma only |
| | | | collection post-dose |
| | 6 | 44 | None; euthanized for end of study |
| | 7 | 44 | None; euthanized for end of study |
| | 8 | 43 | None; euthanized for end of study 8 h plasma only |
| | | | collection post-dose |
| Group 10 | 1 | 36 | None; euthanized for end of study |
| Compound 2 | 2 | 36 | None; euthanized for end of study |
| 300 mg/kg | 3 | 36 | None; euthanized for end of study 8 h plasma only |
| Q3Dx11 | | | collection post-dose |
| | 4 | 36 | None; euthanized for end of study 8 h plasma only |
| | | | collection post-dose |
| | 5 | 36 | None; euthanized for end of study 8 h plasma only |
| | | | collection post-dose |
| | 6 | 36 | None; euthanized for end of study |
| | 7 | 36 | None; euthanized for end of study |
| | 8 | 36 | None; euthanized for end of study |

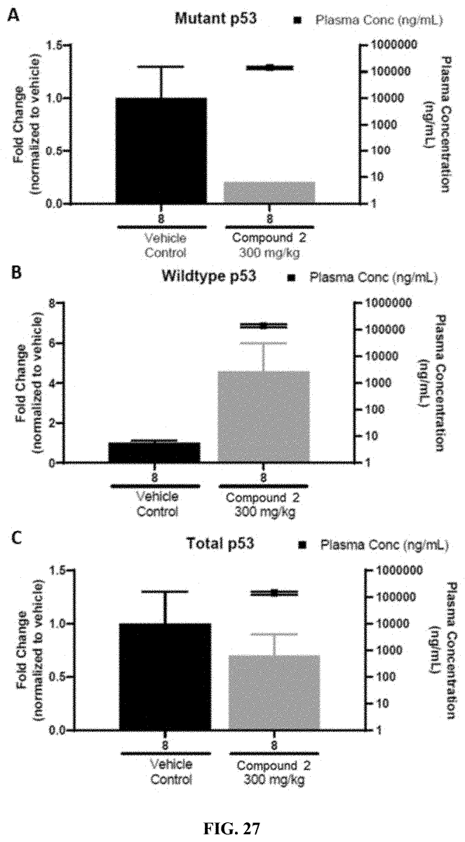

End of Efficacy PK/PD Results: Four mice, treated with Compound 2 at 300 mg/kg Q3D×11, with sufficiently large tumors to isolate protein had tumor and plasma harvested for PD/PK analysis at 8 h post the final dose. Four animals treated with Compound 2 at 100 mg/kg QD×44 had plasma only harvested 8 h post final dose. Tumors from mice dosed with Compound 2 at 300 mg/kg resulted in a 4.6-fold increase in WT conformation p53, an 80% decrease in mutant p53 and a 30% decrease in total levels of p53. Plasma concentrations of Compound 2 from the 300 mg/kg and 100 mg/kg groups were 142,133 ng/mL and 113,275 ng/mL 8 h post-dose, respectively. FIG. 27 PANEL A-PANEL C show plasma concentration (ng/mL) and fold changes normalized to vehicle of mutant p53, WT conformation p53, and p53 for mice treated with the vehicle control and 300 mg/kg of Compound 2. TABLE 42 shows numerical values of plasma concentration (ng/mL) of mice treated with 100 mg/kg Compound 2 QD×44 and 300 mg/kg Compound 2 Q3D×11; and fold changes over vehicle control or percent reduction relating to vehicle control of mutant p53, WT conformation p53, total p53, p21, MDM2, and MIC-1.

TABLE 42

| | | Fold Change over Vehicle Control or Percent Reduction | | | | | |
|---|---|---|---|---|---|---|---|
| | | Relative to Vehicle Control (%) | | | | | |
| Group - | Plasma | Mutant | WT | Total | | | |
| Compound 2 | Conc. | p53 | p53 | p53 | p21 | MDM2 | MIC-1 |
| (mg/kg) | (ng/mL) | Protein | Protein | Protein | Protein | Protein | Protein |
| Group 8 - | 113,275 | N/A | N/A | N/A | N/A | N/A | 1.39* |
| 100 QDx44 | | | | | | | |
| Group 10 - | 142,133 | 80.0% | 4.6 | 30.0% | 9.2 | 46.0 | 22.03* |
| 300 Q3Dx11 | | | | | | | |

*= Absolute induction in pg/mL/mm³.

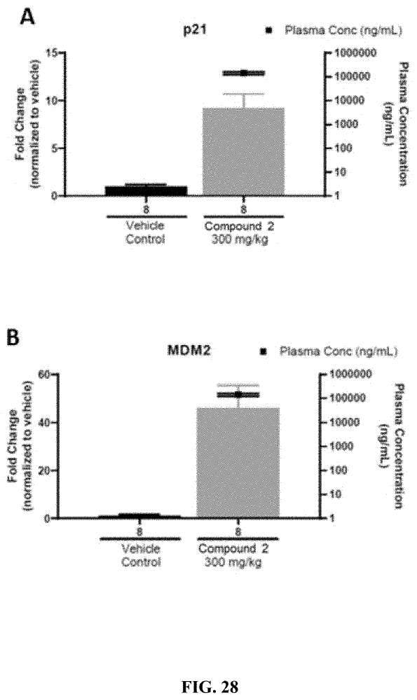

FIG. 28 PANEL A and PANEL B show conversion of mutant to WT conformation p53 results in downstream increases in p53 target proteins: p21 and MDM2 in mice treated with vehicle control or Compound 2 300 mg/kg. Measurement of p53 target proteins downstream of WT p53 demonstrated a 9.2-fold increase in p21 and a 46-fold increase in MDM2 in tumors of mice dosed with 300 mg/kg Q3D×11 at 8 h.

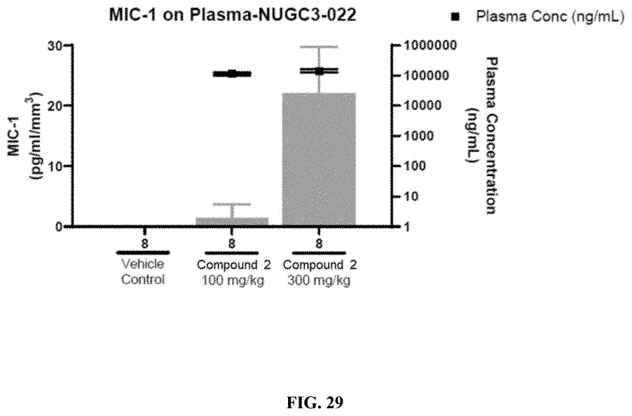

Increases in MIC-1 cytokine levels can be measured in the plasma of the mice and normalized to individual tumor volume. In mice receiving vehicle control MIC-1 levels were undetectable while those administered Compound 2 at 100 mg/kg QD×44 and 300 mg/kg Q3D×11 had 1.39 μg/mL/mm³ and 22.03 μg/mL/mm³ plasma MIC-1 levels at 8 h post-dose, respectively. FIG. 29 shows plasma concentration (ng/mL) and MIC-1 levels (pg/mL/mm³) in mice treated with the vehicle control; 100 mg/kg Compound 2; or 300 mg/kg Compound 2.

On days 23-26 of the study, mice from both Compound 2 dosed groups exhibited tumor regression of 70% (D26) and 76% (D23) for the mice administered 100 mg/kg QD×44 and 300 mg/kg Q3D×11, respectively. Compound 2 was well tolerated with no significant body weight loss across the course of the study. On the day following the final dose, tumors of sufficient size and plasma from mice treated with Compound 2 at 300 mg/kg Q3D×11 or vehicle were collected for PD and PK analysis at 8 h post-dose. The robust tumor regression correlated with a reduction in mutant p53 (80%) and an increase in WT conformation p53 (4.6-fold) when compared to vehicle treated tumors. Protein levels of p53 target genes in the tumor (p21 and MDM2) and plasma (MIC-1) were also increased compared to control.

### Example 9: Measurement of the Pharmacodynamic and PK Response to Compound 2 in a Mouse Xenograft Model of Gastric Cancer (NUGC3) when Treated with 300 mg/kg BID×1 or 100 mg/kg QD×6

The PD and PK relationship of Compound 2 administered at two dose levels and regimens were tested in a mouse xenograft model of gastric cancer (NUGC3). Mice bearing p53 Y220C mutant NUGC3 tumor xenografts were administered with either vehicle (0.2% hydroxypropyl cellulose (HPC), 0.5% Tween 80) or Compound 2 orally (PO) at 300 mg/kg twice in a single day (BID×1) 8 h apart, or 100 mg/kg daily for six days (QD×6). Tumors and plasma samples were harvested at 7, 12, 24, 48, 72, 96, 120, and 144 h for the 300 mg/kg BID×1 group and 8, 16, 24, 32, 48, 80, 96, 128, and 144 h for the 100 mg/kg QD×6 group. Plasma samples were analyzed for Compound 2 levels by LC/MS-MS. Tumors were analyzed for mutant (MUT), wild-type (WT), and total p53 protein levels and downstream induction of p53 target gene transcription and protein levels as evidence of target engagement. TABLE 43 shows the treatment groups, dosing regimens, and harvest timepoints of the study.

TABLE 43

| | | | | Dosing | | |
|---|---|---|---|---|---|---|
| | | | | Frequency & | Dose | Harvest timepoints |
| Group | Treatment | N | Route | Duration | (mg/kg) | (h post last dose) |
| 1 | Vehicle Control | 12 | PO | BIDx1 | N/A | 7, 24, 48 |
| | | (4/tp) | | | | |
| 2 | Compound 2 | 32 | PO | BIDx1 | 300 | 7, 12, 24, 48, 72, 96, |
| | | (4/tp) | | | | 120, 144 |
| 3 | Compound 2 | 36 | PO | QDx6 | 100 | 8, 16, 24, 32, 48, 80, |
| | | (4/tp) | | | | 96, 128, 144 |

Animals: Female Balb/c nude mice (200 total) were purchased from Envigo acclimatized for 1 week. The animals were 8-10 weeks old at initiation of study. Animals were group housed (N=5) in ventilated cages and cared for as descried in EXAMPLE 5. Implantation of tumor cells into animals was performed as described in EXAMPLE 7.

Tumor Cell Culture: NUGC3 cells were cultured in RPMI medium with 10% fetal bovine serum. The cells were washed with PBS and counted at a total of 2.25×10⁸ cells with 96.7% viability. Cells were centrifuged and resuspended in 50% PBS:50% Matrigel Matrix at a concentration of 1×10⁸ viable cells/100 μL.

Randomization and Study Setup: Implanted animals were monitored for palpable tumors. Fifteen days post implant the animals with palpable tumors had their tumor sizes determined via digital caliper. Mice were selected and randomized into three treatment groups, according to tumor size. Treatment began on the 16^{th} day to facilitate the collection schedule. TABLE 44 shows the average tumor volume (mm³) and body weight (g) of the treatment groups.

TABLE 44

| | | Tumor Volume (mm³) | | | | Body Weight (g) | | | |
|---|---|---|---|---|---|---|---|---|---|
| Group | N | Mean | St Dev | Min | Max | Mean | St Dev | Min | Max |
| 1 - Vehicle Control | 12 | 328.86 | ±107.77 | 176.82 | 447.53 | 23.81 | ±2.04 | 19.90 | 26.70 |
| 2 - Compound 2 at | 32 | 345.22 | ±84.76 | 201.39 | 522.38 | 24.21 | ±2.28 | 19.30 | 28.20 |
| 300 mg/kg BIDx1 | | | | | | | | | |
| 3 - Compound 2 at | 36 | 371.74 | ±81.78 | 207.92 | 534.26 | 24.31 | ±1.75 | 20.70 | 27.90 |
| 100 mg/kg QDx6 | | | | | | | | | |

Measurements and Calculation of Tumor Volume; and tumor lysate preparation were performed as described in EXAMPLE 5. The mutant p53, WT p53, and total p53 ELISA; and the p21 and MDM2 ELISA were performed as described in EXAMPLE 7.

Plasma MIC-1 ELISA: On day 1, a 96-well plate was coated with 100 μL of capture antibody diluted in PBS at working concentration. The plate was sealed and incubated overnight at room temperature. On the second day, each well was washed with wash buffer 3×. The plate was blocked with blocking buffer for 1 h at room temperature, after which the plate was washed 3× with wash buffer. 100 μL of plasma samples (at appropriate dilutions depending on the tumor model and treatments) or standards were added to the plate, and the plate was covered and incubated for 2 h at room temperature. The plate was washed 3×, and 100 μL of detection antibody was added and incubated for 2 h at room temperature. The plate was washed again, and 100 μL of Streptavidin-HRP was added. The plate was incubated further for 20 minutes at room temperature. The plate was again washed, and 100 μL of Substrate Solution added for 10 minutes at room temperature. 50 μL of stop solution was added, and the plate was gently shaken to ensure thorough mixing prior to reading the plate at 450/540 nm. Plasma MIC-1 levels were calculated using the standard curve and dilution factors.

p21, MDM2, BRIC5, and GAPDH Gene Expression: Frozen tumor samples were lysed in Buffer RLT with 10 μL/mL β-mercaptoethanol in a TissueLyser. Total RNA was purified from the lysate by QIAcube with DNase digestion. RNA concentrations were measured using a NanoDrop 2000 Spectrophotometer. Purified total RNA was diluted to 2.5 ng/μL in DNase-free and RNase-free water, and 10 ng was used for each TaqMan-based one-step RT-qPCR assay in 20 μL reaction using LightCycler 96. For each assay, QuantiTect Probe RT-PCR Kit was used along with p21, MDM2, BIRC5, or GAPDH primer/probe sets. Gene expression of p21, MDM2, or BIRC5 relative to GAPDH was calculated by the ΔCₜ method, and then the gene expression of p21/GAPDH, MDM2/GAPDH, or BIRC5/GAPDH was normalized to vehicle control by calculating the ΔΔCₜ.

Human p53 Signaling Pathway Profiling: RNA was extracted and quantified as described above. Human p53 signaling pathway profiling was performed using SYBR Green-based real-time qPCR after the reverse transcription. In brief, the first strand cDNA was synthesized from 500 ng purified total RNA of each tumor sample by the RT2 First Strand Kit before being mixed with RT2 SYBR Green qPCR Mastermix. The mixture was then applied to an RT² Profiler™ PCR Array Human p53 Signaling Pathway plate and detected by LightCycler 96. At least 3 samples in each group were used for the profiling. Data were analyzed after uploading Cₜ values of profiled genes resulted from all groups of samples, using the average Cₜ values of 5 housekeeping genes on the plate as the reference control to normalize inter-plate variation. Alternatively, a similar result was achieved by the ΔΔCₜ method using 5 housekeeping genes as the first reference control and the vehicle group as the second reference control. Finally, the cut-off of fold change=2 and p-value=0.05 was applied to curate the data, with a consideration of eliminating some low expression genes (Cₜ<30).

PDs and PK of Compound 2: Administration of Compound 2 at 300 mg/kg BID×1 resulted in a maximum 96% reduction in mutant p53, compared to vehicle control tumors, 12 h post-dose when plasma concentration reached 69,550 ng/mL and remained reduced (83-95%) until 120 h when plasma concentration reduced to 103 ng/mL. Administration of Compound 2 at 100 mg/kg QD×6 resulted in an 87% reduction in mutant p53 at 8 h post dose initial dose. Continued daily administration resulted in reductions of mutant p53 of 93%, 96%, and 94% at 8 h post dose on days 2, 4, and 6, respectively, correlating with peak plasma concentrations of approximately 37,000 ng/mL. Administration of 100 mg/kg QD×6 resulted in an immediate reduction (73%) of total p53 levels that was sustained (65-95%) through 24 h on day 6

Administration of Compound 2 at 300 mg/kg BID×1 resulted in 3.2 and 1.8-fold increases in WT conformation p53 at 7 and 12 h, respectively, when plasma concentrations were approximately 61,000 ng/mL and returned to baseline thereafter. Daily administration of Compound 2 at 100 mg/kg resulted in WT conformation p53 increases of 4.5-, 3.3-, and 3.1-fold at 8 h post-dose on days 1, 2, and 6, respectively, that decreased to baseline levels when plasma concentrations fell below approximately 10,000 ng/mL. Levels of total p53 were immediately reduced 87% by 7 h in the Compound 2 300 mg/kg BID×1 group and this was sustained (81-93%) through 96 h, after plasma concentrations lowered to 103 ng/mL.

Measurement of p53 target protein levels in tumors from mice administered 300 mg/kg Compound 2 BID×1 resulted in maximum increases of 7.3- and 99.0-fold in MDM2 and p21, respectively, at 7 h post dose on day 1 that steadily returned to baseline by 120 h which correlated with plasma concentrations of 53,375 ng/mL at 7 h that reduced to approximately 103 ng/mL by 120 h. Tumors from mice administered 100 mg/kg Compound 2 QD×6 resulted in increases of 4.7- and 27.9-fold in MDM2 and p21, respectively, at 8 h post dose on day 1. With daily dosing, Compound 2 plasma concentrations peaked daily (approximately 37,000 ng/mL by 8 h), and likewise MDM2 levels increased 7.3-, 3.4-, and 4.8-fold and p21 levels increased 76.4-, 19.7-, and 16.7-fold 8 h post-dose on days 2, 4, and 6, respectively.

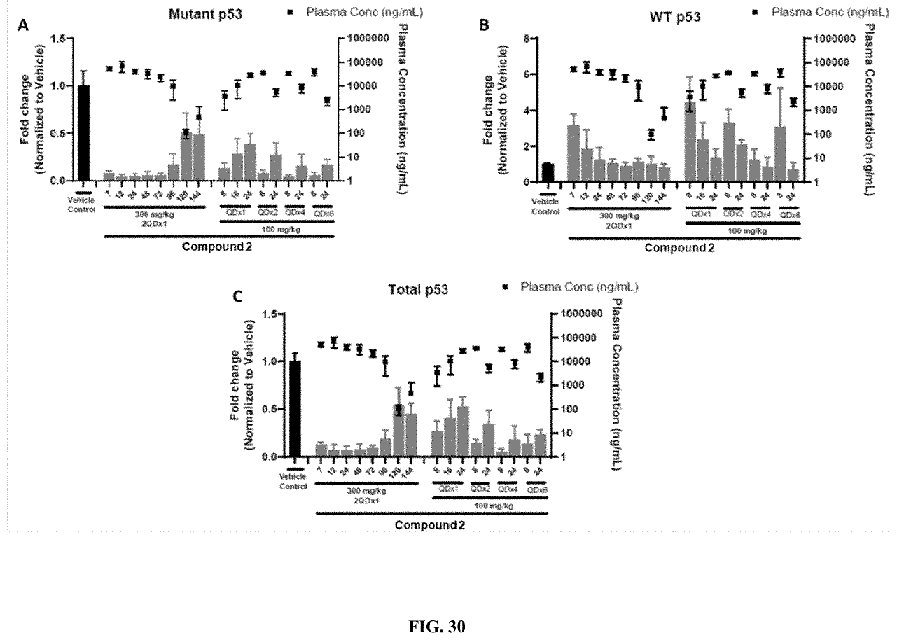

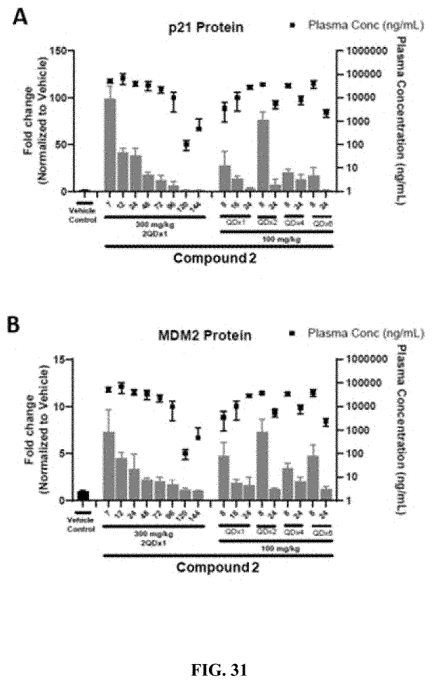

FIG. 30 PANEL A-PANEL C show conversion of mutant p53 to wild-type conformation in mice treated with vehicle control. Compound 2 300 mg/kg 2QD×1, 100 mg/kg QD×1, QD×2, QD×4, or QD×6. FIG. 31 PANEL A and PANEL B show fold changes normalized to vehicle and plasma concentration (ng/mL) of p21 protein and MDM2 protein in mice treated with vehicle control; Compound 2 300 mg/kg (2QD×1); and Compound 2 100 mg/kg (QD×1, QD×3, QD×4, and QD×6). TABLE 45 shows time points of sample collection, plasma concentration (ng/mL), and fold changes over vehicle control or percent reduction relative to vehicle control in mutant p53 protein, WT conformation p53 protein, total p53 protein, p21 protein, MDM2 protein, p21 mRNA, MDM2 mRNA, BIRC5 mRNA, and MIC-1 plasma in mice treated with Compound 2 300 mg/kg BID×1 and Compound 2 100 mg/kg QD×6.

TABLE 45

| | | | Fold Change over Vehicle Control or Percent Reduction Relative to | | | | | | | | |
|---|---|---|---|---|---|---|---|---|---|---|---|
| | | | Vehicle Control (%) | | | | | | | | |
| Group - | | Plasma | Mutant | WT | Total | | | | | | |
| Compound 2 | Timepoint | Conc. | p53 | p53 | p53 | p21 | MDM2 | p21 | MDM2 | BIRC5 | MIC-1 |
| (mg/kg) | (h) | (ng/mL) | Protein | Protein | Protein | Protein | Protein | mRNA | mRNA | mRNA | Plasma* |
| Group 2 - | 7 | 53375 | 92.3% | 3.17 | 87.3% | 98.95 | 7.32 | 11.44 | 12.90 | 69.4% | 11.08 |
| 300 BIDx1 | 12 | 69550 | 96.2% | 1.84 | 93.0% | 41.43 | 4.49 | 13.52 | 12.61 | 79.8% | 6.45 |
| | 24 | 40700 | 95.4% | 1.23 | 92.7% | 38.51 | 3.40 | 9.66 | 7.77 | 96.3% | 2.55 |
| | 48 | 34625 | 94.6% | 1.04 | 92.1% | 17.32 | 2.20 | 15.86 | 12.08 | 98.8% | 2.41 |
| | 72 | 22725 | 94.3% | 11.4% | 90.8% | 12.14 | 2.07 | 13.77 | 9.15 | 98.3% | 1.05 |
| | 96 | 9818 | 83.1% | 1.11 | 81.0% | 5.84 | 1.70 | 7.07 | 5.55 | 95.5% | 0.70 |
| | 120 | 103 | 49.7% | 1.01 | 46.2% | 0.97 | 1.13 | 1.60 | 1.79 | 49.2% | 0.03\hat{ }\hat{ } |
| | 144 | 474 | 51.6% | 21.1% | 54.6% | 0.95 | 1.05 | 1.66 | 1.40 | 55.2% | 0.02\hat{ }\hat{ } |
| Group 3 - | 8 | 3550 | 86.6% | 4.46 | 72.6% | 27.89 | 4.73 | 10.22 | 11.09 | 63.5% | 6.93 |
| 100 QDx6 | 16 | 10010 | 71.5% | 2.37 | 59.6% | 13.29 | 1.89 | 3.77 | 2.86 | 73.2% | 1.46 |
| | 24 | 28550 | 61.5% | 1.38 | 47.2% | 2.51 | 1.59 | 2.11 | 1.45 | 85.6% | 0.43 |
| | 32 | 37100 | 92.5% | 3.32 | 85.8% | 76.43 | 7.29 | 12.71 | 14.40 | 90.4% | 7.22 |
| | 48 | 5560 | 72.9% | 2.08 | 65.2% | 6.48 | 1.20 | 3.40 | 2.98 | 93.3% | 0.99 |
| | 80 | 33750 | 95.9% | 1.26 | 94.9% | 19.68 | 3.41 | 16.25 | 17.11 | 93.6% | 1.62 |
| | 96 | 8325 | 84.7% | 14.4% | 82.2% | 12.77 | 2.04 | 6.34 | 6.04 | 94.0% | 1.03 |
| | 128 | 39375 | 94.3% | 3.08 | 85.9% | 16.68 | 4.75 | 14.22 | 14.42 | 92.7% | 2.29 |
| | 144 | 2318 | 83.0% | 29.8% | 76.9% | 1.32 | 1.21 | 1.88 | 2.20 | 93.3% | 0.26 |

*Absolute MIC-1 values normalized to tumor volume (pg/mL/mm³).
\hat{ }\hat{ }negative values resulting from background subtraction.

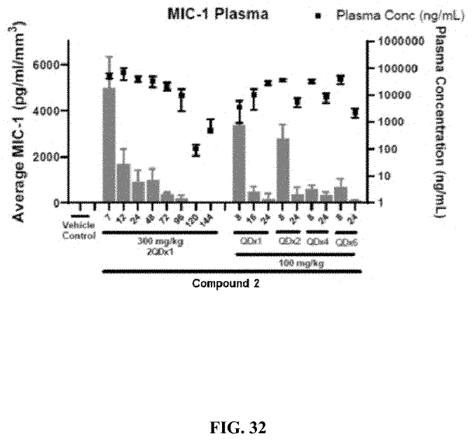

MIC-1 is a p53 target gene, a protein made in the tumor and secreted into the blood that can be measured in the plasma of mice (normalized to tumor volume) as a circulating biomarker. Mice administered with Compound 2 at 300 mg/kg BID×1 had peak elevated MIC-1 levels in the plasma of 11.1 μg/mL/mm³ 7 h post-dose, which levels reduced to undetectable levels by 120 h. The MIC-1 modulation correlated with plasma exposure being approximately 53,375 ng/mL by 7 h post-dose and reducing to 103 ng/mL by 120 h. Administration of 100 mg/kg Compound 2 QD×6 resulted in increases of plasma MIC-1 levels to 6.9, 7.2, 46, and 2.3 μg/mL/mm³ at 8 h post dose on days 1, 2, 4, and 6, respectively, consistent with peak plasma exposures of Compound 2 (approximately 37,000 ng/mL) at the corresponding time points. FIG. 32 shows average MIC-1 (pg/mL/mm³) and plasma concentration (ng/mL) of mice treated with vehicle control, 300 mg/kg Compound 2 (2QD×1), and 100 mg/kg Compound 2 (QD×1, QD×2, QD×4, and QD×6).

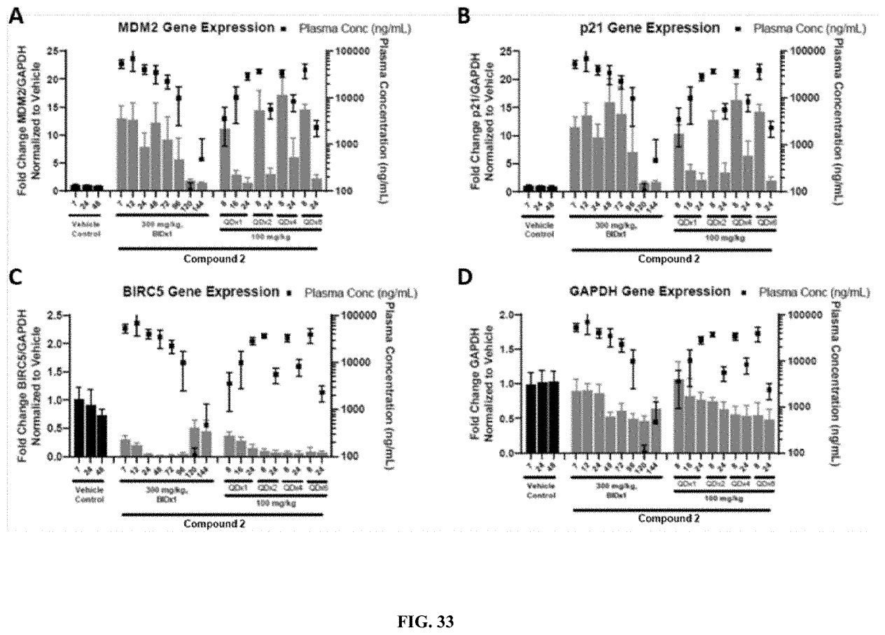

p53 dependent gene expression changes were analyzed initially for three p53 target gene mRNAs; p21, MDM2 and BIRC (Survivin). Administration of Compound 2 at 300 mg/kg BID×1 resulted in increases of 11.4 and 12.9-fold for p21 and MDM2, respectively, at 7 h. The levels were sustained (7.0-15.9-fold and 5.6-12.6-fold, respectively) through 120 h and thereafter returned to baseline as plasma exposure fell to 103 ng/mL. A reduction in BIRC5 (96-99%) was recorded between 24 and 96 h as Compound 2 plasma exposure averaged 27,000 ng/mL. Administration of Compound 2 at 100 mg/kg resulted in increases of 10.2 and 11.1-fold in p21 and MDM2, respectively at 8 h post-dose falling to near baseline levels (2.1-1.5-fold) by 24 h. Repeat dosing with Compound 2 at 100 mg/kg resulted in increases of 12.7, 16.3, and 14.2-fold for p21 and 14.4, 17.1, and 14.4-fold for MDM2 at 8 h on day 2, day 4, and day 6, respectively, as plasma concentration reached 36,600 ng/mL. An 85.6% reduction in BIRC5 mRNA expression was observed at 24 h following initial dose and sustained this decrease (90.4-94.0%) through to day 6. FIG. 33 PANEL A-PANEL D show fold change normalized to vehicle and plasma concentration (ng/mL) of MDM2, p21, BIRC5, and GAPDH gene expression in mice treated with vehicle control, 300 mg/kg Compound 2 (BID×1), and 100 mg/kg Compound 2 (QD×1, QD×2, QD×4, and QD×6).

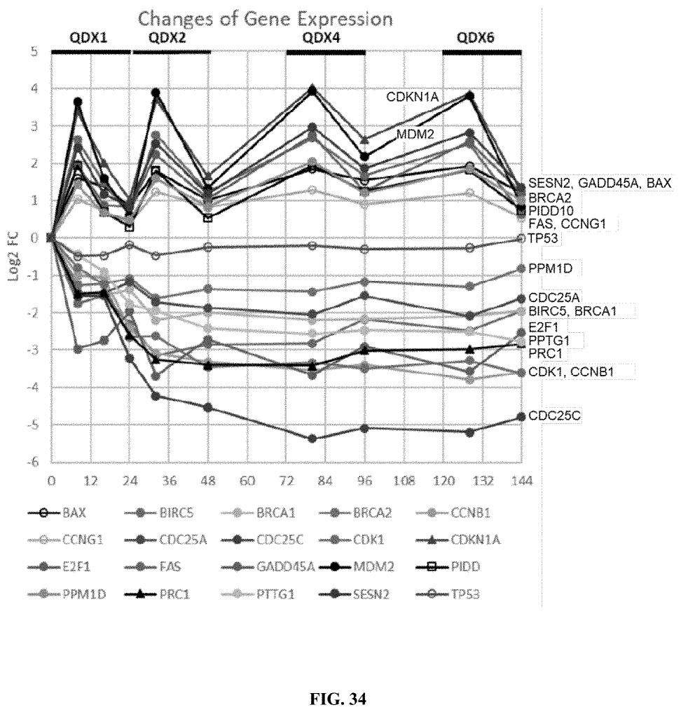

A larger panel of p53 target genes were assessed to understand gene expression changes across time in the tumors from mice dosed with 100 mg/kg Compound 2 QD×6. Following administration of Compound 2 at 100 mg/kg QD×6, the largest fold mRNA expression change occurred 8 h post-dose with the changes returning toward baseline by 24 h post-dose. A representative 20 out of 84 genes are shown in FIG. 34 and TABLE 46 to illustrate the p53-related pathway gene expression changes. Levels of p53 mRNAs did not change across the course of the study. Gene expression increases were measured immediately following administration of 100 mg/kg with peak expression changes occurring approximately 8 h dose at all days measured. Two mRNAs exhibiting the largest fold increases were p21 (CDKN1A; 10.6-16.4-fold) and MDM2 (12.6-15.2-fold). Decreases in selected genes mRNA expression were observed at 24 h post the first dose of 100 mg/kg Compound 2. Some of the largest fold gene expression decreases measured included CDC25C, PRC1, CDK1, and CCNB1 that were reduced by 88%, 83%, 83%, and 81%, respectively. Negatively regulated genes expression levels did not return to baseline 24 h post-dose, unlike positively regulated gene expression, and remained reduced throughout the course of dosing.

In conclusion, administration of Compound 2 at 300 mg/kg BID×1 and 100 mg/kg QD×6 to mice bearing NUGC3 xenografts resulted in dose proportional exposures of Compound 2 leading to quick restoration of WT conformation p53 and activation of downstream transcriptional targets and subsequently proteins. Repeat administration of Compound 2 resulted in daily waves of these WT conformation p53 restoration patterns that correlated well with repeat exposure of Compound 2 in the blood.

TABLE 46

| | | Compound 2 100 mg/kg QDx6 Fold Change Compared to Vehicle Control | | | | | | | | |
|---|---|---|---|---|---|---|---|---|---|---|
| | Vehicle | QDx6 | QDx6 | QDx6 | QDx6 | QDx6 | QDx6 | QDx6 | QDx6 | QDx6 |
| Gene Name | Control | 8 h | 16 h | 24 h | 32 h | 48 h | 80 h | 96 h | 128 h | 144 h |
| ARAF1 | 1.00 ± | 1.23 ± | 1.11 ± | 1.21 ± | 1.43 ± | 1.17 ± | 1.51 ± | 1.43 ± | 1.37 ± | 1.51 ± |
| | 0.07 | 0.12 | 0.08 | 0.12 | 0.09 | 0.07 | 0.07 | 0.15 | 0.16 | 0.10 |
| ATM | 1.00 ± | 0.81 ± | 1.11 ± | 1.51 ± | 1.45 ± | 1.34 ± | 1.51 ± | 1.87 ± | 1.62 ± | 1.96 ± |
| | 0.08 | 0.08 | 0.07 | 0.11 | 0.10 | 0.08 | 0.13 | 0.31 | 0.07 | 0.08 |
| ATR | 1.00 ± | 0.81 ± | 1.02 ± | 1.15 ± | 1.05 ± | 1.09 ± | 1.14 ± | 1.33 ± | 1.13 ± | 1.50 ± |
| | 0.05 | 0.08 | 0.06 | 0.06 | 0.05 | 0.06 | 0.07 | 0.14 | 0.06 | 0.05 |
| BAL1 | 1.00 ± | 1.31 ± | 1.47 ± | 2.29 ± | 2.03 ± | 2.41 ± | 3.16 ± | 3.72 ± | 3.63 ± | 4.13 ± |
| | 0.27 | 0.26 | 0.26 | 0.49 | 0.32 | 0.40 | 0.46 | 0.72 | 0.54 | 0.48 |
| BAX | 1.00 ± | 3.04 ± | 2.63 ± | 1.84 ± | 3.47 ± | 2.03 ± | 3.58 ± | 2.94 ± | 3.78 ± | 2.36 ± |
| | 0.05 | 0.16 | 0.19 | 0.29 | 0.19 | 0.28 | 0.18 | 0.26 | 0.04 | 0.40 |
| BBC3 | 1.00 ± | 3.85 ± | 1.89 ± | 1.44 ± | 3.75 ± | 2.51 ± | 5.80 ± | 2.39 ± | 5.16 ± | 2.50 ± |
| | 0.16 | 0.29 | 0.23 | 0.29 | 0.20 | 0.56 | 0.74 | 0.33 | 0.33 | 0.47 |
| BCL2 | 1.00 ± | 0.74 ± | 0.98 ± | 0.99 ± | 0.86 ± | 1.04 ± | 1.64 ± | 1.77 ± | 1.76 ± | 2.16 ± |
| | 0.14 | 0.12 | 0.15 | 0.13 | 0.11 | 0.16 | 0.49 | 0.32 | 0.45 | 0.29 |
| BCL2A1 | 1.00 ± | 2.08 ± | 1.04 ± | 0.84 ± | 0.78 ± | 1.30 ± | 0.64 ± | 1.19 ± | 1.83 ± | 1.20 ± |
| | 0.40 | 1.13 | 0.39 | 0.47 | 0.35 | 0.53 | 0.34 | 0.57 | 0.99 | 0.59 |
| BID | 1.00 ± | 0.87 ± | 0.92 ± | 1.03 ± | 0.87 ± | 1.10 ± | 1.06 ± | 1.11 ± | 1.16 ± | 1.25 ± |
| | 0.08 | 0.09 | 0.06 | 0.08 | 0.08 | 0.09 | 0.13 | 0.11 | 0.07 | 0.08 |
| BIRC5 | 1.00 ± | 0.30 ± | 0.35 ± | 0.21 ± | 0.11 ± | 0.15 ± | 0.14 ± | 0.22 ± | 0.19 ± | 0.26 ± |
| | 0.17 | 0.14 | 0.14 | 0.14 | 0.13 | 0.14 | 0.14 | 0.14 | 0.14 | 0.14 |
| BRCA1 | 1.00 ± | 0.37 ± | 0.35 ± | 0.42 ± | 0.22 ± | 0.25 ± | 0.22 ± | 0.23 ± | 0.24 ± | 0.26 ± |
| | 0.13 | 0.11 | 0.10 | 0.16 | 0.10 | 0.11 | 0.10 | 0.11 | 0.11 | 0.11 |
| BRCA2 | 1.00 ± | 0.42 ± | 0.43 ± | 0.49 ± | 0.33 ± | 0.39 ± | 0.37 ± | 0.45 ± | 0.41 ± | 0.57 ± |
| | 0.09 | 0.08 | 0.07 | 0.12 | 0.08 | 0.08 | 0.07 | 0.09 | 0.08 | 0.09 |
| BTG2 | 1.00 ± | 14.50 ± | 2.70 ± | 1.51 ± | 13.57 ± | 3.59 ± | 19.08 ± | 3.40 ± | 16.31 ± | 2.10 ± |
| | 0.25 | 2.93 | 0.77 | 0.46 | 0.70 | 0.85 | 3.45 | 0.64 | 3.34 | 0.33 |
| ARAF1 | 1.00 ± | 1.23 ± | 1.11 ± | 1.21 ± | 1.43 ± | 1.17 ± | 1.51 ± | 1.43 ± | 1.37 ± | 1.51 ± |
| | 0.07 | 0.12 | 0.08 | 0.12 | 0.09 | 0.07 | 0.07 | 0.15 | 0.16 | 0.10 |
| ATM | 1.00 ± | 0.81 ± | 1.11 ± | 1.51 ± | 1.45 ± | 1.34 ± | 1.51 ± | 1.87 ± | 1.62 ± | 1.96 ± |
| | 0.08 | 0.08 | 0.07 | 0.11 | 0.10 | 0.08 | 0.13 | 0.31 | 0.07 | 0.08 |
| ATR | 1.00 ± | 0.81 ± | 1.02 ± | 1.15 ± | 1.05 ± | 1.09 ± | 1.14 ± | 1.33 ± | 1.13 ± | 1.50 ± |
| | 0.05 | 0.08 | 0.06 | 0.06 | 0.05 | 0.06 | 0.07 | 0.14 | 0.06 | 0.05 |
| BAL1 | 1.00 ± | 1.31 ± | 1.47 ± | 2.29 ± | 2.03 ± | 2.41 ± | 3.16 ± | 3.72 ± | 3.63 ± | 4.13 ± |
| | 0.27 | 0.26 | 0.26 | 0.49 | 0.32 | 0.40 | 0.46 | 0.72 | 0.54 | 0.48 |
| BAX | 1.00 ± | 3.04 ± | 2.63 ± | 1.84 ± | 3.47 ± | 2.03 ± | 3.58 ± | 2.94 ± | 3.78 ± | 2.36 ± |
| | 0.05 | 0.16 | 0.19 | 0.29 | 0.19 | 0.28 | 0.18 | 0.26 | 0.04 | 0.40 |
| BBC3 | 1.00 ± | 3.85 ± | 1.89 ± | 1.44 ± | 3.75 ± | 2.51 ± | 5.80 ± | 2.39 ± | 5.16 ± | 2.50 ± |
| | 0.16 | 0.29 | 0.23 | 0.29 | 0.20 | 0.56 | 0.74 | 0.33 | 0.33 | 0.47 |
| BCL2 | 1.00 ± | 0.74 ± | 0.98 ± | 0.99 ± | 0.86 ± | 1.04 ± | 1.64 ± | 1.77 ± | 1.76 ± | 2.16 ± |
| | 0.14 | 0.12 | 0.15 | 0.13 | 0.11 | 0.16 | 0.49 | 0.32 | 0.45 | 0.29 |
| BCL2A1 | 1.00 ± | 2.08 ± | 1.04 ± | 0.84 ± | 0.78 ± | 1.30 ± | 0.64 ± | 1.19 ± | 1.83 ± | 1.20 ± |
| | 0.40 | 1.13 | 0.39 | 0.47 | 0.35 | 0.53 | 0.34 | 0.57 | 0.99 | 0.59 |
| BID | 1.00 ± | 0.87 ± | 0.92 ± | 1.03 ± | 0.87 ± | 1.10 ± | 1.06 ± | 1.11 ± | 1.16 ± | 1.25 ± |
| | 0.08 | 0.09 | 0.06 | 0.08 | 0.08 | 0.09 | 0.13 | 0.11 | 0.07 | 0.08 |
| BIRC5 | 1.00 ± | 0.30 ± | 0.35 ± | 0.21 ± | 0.11 ± | 0.15 ± | 0.14 ± | 0.22 ± | 0.19 ± | 0.26 ± |
| | 0.17 | 0.14 | 0.14 | 0.14 | 0.13 | 0.14 | 0.14 | 0.14 | 0.14 | 0.14 |
| BRCA1 | 1.00 ± | 0.37 ± | 0.35 ± | 0.42 ± | 0.22 ± | 0.25 ± | 0.22 ± | 0.23 ± | 0.24 ± | 0.26 ± |
| | 0.13 | 0.11 | 0.10 | 0.16 | 0.10 | 0.11 | 0.10 | 0.11 | 0.11 | 0.11 |
| BRCA2 | 1.00 ± | 0.42 ± | 0.43 ± | 0.49 ± | 0.33 ± | 0.39 ± | 0.37 ± | 0.45 ± | 0.41 ± | 0.57 ± |
| | 0.09 | 0.08 | 0.07 | 0.12 | 0.08 | 0.08 | 0.07 | 0.09 | 0.08 | 0.09 |
| BTG2 | 1.00 ± | 14.50 ± | 2.70 ± | 1.51 ± | 13.57 ± | 3.59 ± | 19.08 ± | 3.40 ± | 16.31 ± | 2.10 ± |
| | 0.25 | 2.93 | 0.77 | 0.46 | 0.70 | 0.85 | 3.45 | 0.64 | 3.34 | 0.33 |
| CASP2 | 1.00 ± | 0.62 ± | 0.77 ± | 1.06 ± | 0.81 ± | 0.93 ± | 0.99 ± | 1.18 ± | 1.02 ± | 1.48 ± |
| | 0.09 | 0.08 | 0.07 | 0.10 | 0.07 | 0.08 | 0.09 | 0.12 | 0.07 | 0.10 |
| CASP9 | 1.00 ± | 0.83 ± | 1.07 ± | 1.32 ± | 1.20 ± | 1.42 ± | 1.57 ± | 1.76 ± | 1.72 ± | 2.10 ± |
| | 0.09 | 0.07 | 0.07 | 0.08 | 0.10 | 0.08 | 0.15 | 0.13 | 0.11 | 0.11 |
| CCNB1 | 1.00 ± | 0.50 ± | 0.49 ± | 0.19 ± | 0.12 ± | 0.10 ± | 0.09 ± | 0.10 ± | 0.08 ± | 0.08 ± |
| | 0.17 | 0.15 | 0.16 | 0.14 | 0.13 | 0.13 | 0.13 | 0.14 | 0.14 | 0.13 |
| CCNE1 | 1.00 ± | 0.40 ± | 0.56 ± | 0.79 ± | 0.52 ± | 0.63 ± | 0.58 ± | 0.72 ± | 0.61 ± | 0.77 ± |
| | 0.04 | 0.05 | 0.03 | 0.16 | 0.04 | 0.05 | 0.06 | 0.06 | 0.05 | 0.05 |
| CCNG1 | 1.00 ± | 2.06 ± | 1.65 ± | 1.34 ± | 2.35 ± | 1.76 ± | 2.44 ± | 1.86 ± | 2.30 ± | 1.47 ± |
| | 0.05 | 0.11 | 0.08 | 0.17 | 0.10 | 0.09 | 0.09 | 0.07 | 0.09 | 0.08 |
| CCNH | 1.00 ± | 0.98 ± | 1.09 ± | 1.16 ± | 1.28 ± | 1.15 ± | 1.27 ± | 1.39 ± | 1.38 ± | 1.43 ± |
| | 0.05 | 0.10 | 0.06 | 0.09 | 0.06 | 0.05 | 0.06 | 0.13 | 0.06 | 0.09 |
| CDC25A | 1.00 ± | 0.36 ± | 0.36 ± | 0.48 ± | 0.30 ± | 0.28 ± | 0.24 ± | 0.34 ± | 0.24 ± | 0.33 ± |
| | 0.12 | 0.10 | 0.10 | 0.19 | 0.10 | 0.11 | 0.10 | 0.10 | 0.10 | 0.10 |
| CDC25C | 1.00 ± | 0.35 ± | 0.35 ± | 0.12 ± | 0.06 ± | 0.05 ± | 0.03 ± | 0.04 ± | 0.04 ± | 0.04 ± |
| | 0.16 | 0.14 | 0.15 | 0.14 | 0.13 | 0.13 | 0.13 | 0.13 | 0.13 | 0.13 |
| CDK1 | 1.00 ± | 0.58 ± | 0.43 ± | 0.17 ± | 0.17 ± | 0.10 ± | 0.10 ± | 0.09 ± | 0.12 ± | 0.08 ± |
| | 0.14 | 0.13 | 0.14 | 0.13 | 0.12 | 0.12 | 0.12 | 0.12 | 0.12 | 0.12 |
| CDK4 | 1.00 ± | 0.69 ± | 0.77 ± | 0.80 ± | 0.65 ± | 0.67 ± | 0.69 ± | 0.70 ± | 0.69 ± | 0.77 ± |
| | 0.09 | 0.08 | 0.07 | 0.13 | 0.07 | 0.08 | 0.08 | 0.11 | 0.08 | 0.07 |
| CDKN1A | 1.00 ± | 10.58 ± | 4.12 ± | 2.23 ± | 13.07 ± | 3.45 ± | 16.42 ± | 6.30 ± | 14.42 ± | 2.42 ± |
| | 0.07 | 0.92 | 0.58 | 0.80 | 0.71 | 1.04 | 1.10 | 0.78 | 0.36 | 0.52 |
| CDKN2A | 1.00 ± | 0.71 ± | 0.79 ± | 1.26 ± | 1.03 ± | 1.23 ± | 1.37 ± | 1.44 ± | 1.30 ± | 1.78 ± |
| | 0.11 | 0.11 | 0.11 | 0.15 | 0.09 | 0.14 | 0.10 | 0.18 | 0.12 | 0.20 |
| CHEK1 | 1.00 ± | 0.60 ± | 0.65 ± | 0.69 ± | 0.53 ± | 0.52 ± | 0.44 ± | 0.44 ± | 0.34 ± | 0.40 ± |
| | 0.13 | 0.11 | 0.12 | 0.14 | 0.11 | 0.13 | 0.11 | 0.13 | 0.11 | 0.12 |
| CHEK2 | 1.00 ± | 0.71 ± | 0.66 ± | 0.58 ± | 0.61 ± | 0.60 ± | 0.57 ± | 0.60 ± | 0.46 ± | 0.57 ± |
| | 0.14 | 0.12 | 0.12 | 0.12 | 0.11 | 0.13 | 0.12 | 0.12 | 0.12 | 0.12 |
| CRADD | 1.00 ± | 0.97 ± | 1.05 ± | 1.13 ± | 1.15 ± | 1.14 ± | 1.20 ± | 1.41 ± | 1.30 ± | 1.42 ± |
| | 0.07 | 0.10 | 0.07 | 0.10 | 0.12 | 0.06 | 0.08 | 0.08 | 0.08 | 0.09 |
| DNMT1 | 1.00 ± | 0.60 ± | 0.60 ± | 0.66 ± | 0.53 ± | 0.53 ± | 0.55 ± | 0.61 ± | 0.53 ± | 0.68 ± |
| | 0.06 | 0.06 | 0.05 | 0.11 | 0.06 | 0.06 | 0.05 | 0.07 | 0.08 | 0.05 |
| E2F1 | 1.00 ± | 0.13 ± | 0.15 ± | 0.38 ± | 0.08 ± | 0.19 ± | 0.08 ± | 0.15 ± | 0.09 ± | 0.18 ± |
| | 0.16 | 0.13 | 0.13 | 0.26 | 0.13 | 0.16 | 0.13 | 0.14 | 0.13 | 0.13 |
| E2F3 | 1.00 ± | 1.00 ± | 0.97 ± | 1.08 ± | 1.16 ± | 1.02 ± | 1.32 ± | 1.29 ± | 1.28 ± | 1.27 ± |
| | 0.07 | 0.06 | 0.06 | 0.09 | 0.08 | 0.08 | 0.09 | 0.09 | 0.10 | 0.08 |
| EGFR | 1.00 ± | 1.05 ± | 1.14 ± | 1.24 ± | 1.13 ± | 1.06 ± | 1.21 ± | 1.21 ± | 1.21 ± | 1.29 ± |
| | 0.08 | 0.12 | 0.09 | 0.12 | 0.07 | 0.07 | 0.10 | 0.13 | 0.11 | 0.08 |
| EGR1 | 1.00 ± | 1.51 ± | 1.28 ± | 1.26 ± | 1.49 ± | 0.99 ± | 1.78 ± | 1.08 ± | 1.58 ± | 1.30 ± |
| | 0.17 | 0.25 | 0.23 | 0.24 | 0.32 | 0.17 | 0.18 | 0.15 | 0.20 | 0.18 |
| EI24 | 1.00 ± | 1.74 ± | 1.56 ± | 1.30 ± | 1.81 ± | 1.25 ± | 1.67 ± | 1.47 ± | 1.83 ± | 1.38 ± |
| | 0.08 | 0.12 | 0.10 | 0.10 | 0.13 | 0.12 | 0.10 | 0.10 | 0.08 | 0.09 |
| ESR1 | 1.00 ± | 0.98 ± | 1.25 ± | 1.61 ± | 1.28 ± | 1.61 ± | 1.79 ± | 2.20 ± | 1.93 ± | 2.72 ± |
| | 0.10 | 0.09 | 0.14 | 0.32 | 0.09 | 0.17 | 0.20 | 0.16 | 0.27 | 0.13 |
| FADD | 1.00 ± | 0.89 ± | 1.06 ± | 1.15 ± | 0.94 ± | 1.10 ± | 1.20 ± | 1.28 ± | 1.24 ± | 1.37 ± |
| | 0.08 | 0.07 | 0.07 | 0.09 | 0.10 | 0.09 | 0.14 | 0.14 | 0.08 | 0.12 |
| FAS | 1.00 ± | 6.23 ± | 2.81 ± | 1.64 ± | 6.81 ± | 2.39 ± | 6.39 ± | 3.18 ± | 5.77 ± | 1.55 ± |
| | 0.10 | 0.35 | 0.26 | 0.45 | 0.62 | 0.36 | 0.52 | 0.30 | 0.42 | 0.11 |
| FASLG | 1.00 ± | 0.85 ± | 1.39 ± | 1.78 ± | 1.37 ± | 1.65 ± | 2.14 ± | 3.15 ± | 2.63 ± | 3.77 ± |
| | 0.19 | 0.20 | 0.24 | 0.47 | 0.22 | 0.32 | 0.24 | 0.52 | 0.58 | 0.33 |
| FOXO3 | 1.00 ± | 0.86 ± | 1.03 ± | 1.32 ± | 1.20 ± | 1.34 ± | 1.47 ± | 1.52 ± | 1.52 ± | 1.86 ± |
| | 0.09 | 0.11 | 0.11 | 0.10 | 0.08 | 0.08 | 0.11 | 0.14 | 0.09 | 0.11 |
| GADD45A | 1.00 ± | 4.00 ± | 1.86 ± | 1.78 ± | 4.71 ± | 2.10 ± | 6.77 ± | 2.38 ± | 5.97 ± | 2.35 ± |
| | 0.11 | 0.58 | 0.21 | 0.14 | 0.30 | 0.26 | 0.69 | 0.22 | 0.32 | 0.40 |
| GML | 1.00 ± | 1.05 ± | 1.00 ± | 1.00 ± | 1.74 ± | 1.00 ± | 1.25 ± | 1.90 ± | 1.00 ± | 1.69 ± |
| | 0.00 | 0.07 | 0.00 | 0.00 | 0.54 | 0.00 | 0.31 | 0.67 | 0.00 | 0.85 |
| HDAC1 | 1.00 ± | 0.90 ± | 0.91 ± | 0.99 ± | 0.95 ± | 0.97 ± | 0.98 ± | 1.03 ± | 0.95 ± | 1.07 ± |
| | 0.07 | 0.06 | 0.06 | 0.10 | 0.08 | 0.06 | 0.08 | 0.09 | 0.06 | 0.07 |
| HK2 | 1.00 ± | 1.90 ± | 2.09 ± | 0.99 ± | 0.63 ± | 0.89 ± | 0.88 ± | 1.03 ± | 1.07 ± | 1.34 ± |
| | 0.20 | 1.13 | 0.69 | 0.35 | 0.19 | 0.24 | 0.18 | 0.17 | 0.22 | 0.21 |
| IGF1R | 1.00 ± | 0.92 ± | 1.17 ± | 1.40 ± | 1.24 ± | 1.34 ± | 1.68 ± | 1.73 ± | 1.69 ± | 1.91 ± |
| | 0.11 | 0.14 | 0.16 | 0.15 | 0.09 | 0.11 | 0.13 | 0.20 | 0.09 | 0.17 |
| IL6 | 1.00 ± | 1.28 ± | 1.11 ± | 1.13 ± | 0.88 ± | 0.81 ± | 1.10 ± | 0.88 ± | 0.67 ± | 0.94 ± |
| | 0.13 | 0.23 | 0.29 | 0.19 | 0.18 | 0.20 | 0.24 | 0.18 | 0.14 | 0.20 |
| JUN | 1.00 ± | 1.08 ± | 1.16 ± | 1.06 ± | 0.81 ± | 0.93 ± | 1.46 ± | 1.18 ± | 1.40 ± | 1.77 ± |
| | 0.14 | 0.15 | 0.19 | 0.14 | 0.12 | 0.12 | 0.22 | 0.23 | 0.18 | 0.21 |
| KAT2B | 1.00 ± | 1.19 ± | 1.15 ± | 1.37 ± | 1.75 ± | 1.49 ± | 1.98 ± | 1.87 ± | 2.03 ± | 2.10 ± |
| | 0.07 | 0.10 | 0.08 | 0.06 | 0.10 | 0.10 | 0.20 | 0.16 | 0.06 | 0.16 |
| KRAS | 1.00 ± | 0.92 ± | 0.92 ± | 1.09 ± | 1.08 ± | 1.10 ± | 1.28 ± | 1.27 ± | 1.33 ± | 1.41 ± |
| | 0.06 | 0.06 | 0.06 | 0.07 | 0.09 | 0.07 | 0.08 | 0.07 | 0.06 | 0.10 |
| MCL1 | 1.00 ± | 0.94 ± | 1.04 ± | 1.34 ± | 1.17 ± | 1.31 ± | 1.50 ± | 1.66 ± | 1.62 ± | 2.01 ± |
| | 0.04 | 0.06 | 0.05 | 0.06 | 0.04 | 0.07 | 0.11 | 0.12 | 0.06 | 0.08 |
| MDM2 | 1.00 ± | 12.58 ± | 3.08 ± | 1.72 ± | 15.05 ± | 2.72 ± | 15.23 ± | 4.71 ± | 14.00 ± | 1.85 ± |
| | 0.04 | 0.42 | 0.37 | 0.68 | 1.59 | 0.74 | 0.82 | 0.91 | 0.55 | 0.30 |
| MDM4 | 1.00 ± | 1.43 ± | 1.15 ± | 1.24 ± | 2.22 ± | 1.20 ± | 2.03 ± | 2.08 ± | 2.06 ± | 1.94 ± |
| | 0.15 | 0.16 | 0.14 | 0.21 | 0.18 | 0.13 | 0.19 | 0.42 | 0.15 | 0.32 |
| MLH1 | 1.00 ± | 1.22 ± | 1.13 ± | 1.09 ± | 1.65 ± | 1.10 ± | 1.32 ± | 1.42 ± | 1.31 ± | 1.43 ± |
| | 0.34 | 0.28 | 0.39 | 0.43 | 0.37 | 0.28 | 0.42 | 0.59 | 0.37 | 0.32 |
| MSH2 | 1.00 ± | 0.58 ± | 0.75 ± | 0.96 ± | 0.76 ± | 0.85 ± | 0.82 ± | 0.98 ± | 0.88 ± | 1.24 ± |
| | 0.04 | 0.04 | 0.04 | 0.07 | 0.05 | 0.05 | 0.06 | 0.12 | 0.05 | 0.05 |
| MYC | 1.00 ± | 1.17 ± | 1.19 ± | 1.06 ± | 1.21 ± | 0.73 ± | 0.84 ± | 0.94 ± | 0.82 ± | 0.95 ± |
| | 0.09 | 0.24 | 0.09 | 0.09 | 0.10 | 0.11 | 0.09 | 0.15 | 0.13 | 0.07 |
| MYOD1 | 1.00 ± | 1.04 ± | 1.44 ± | 2.14 ± | 1.53 ± | 1.62 ± | 1.96 ± | 3.13 ± | 2.78 ± | 3.23 ± |
| | 0.34 | 0.29 | 0.35 | 0.80 | 0.34 | 0.33 | 0.30 | 0.59 | 0.48 | 0.43 |
| NF1 | 1.00 ± | 1.00 ± | 1.08 ± | 1.23 ± | 1.28 ± | 1.17 ± | 1.32 ± | 1.39 ± | 1.39 ± | 1.51 ± |
| | 0.05 | 0.08 | 0.07 | 0.09 | 0.08 | 0.06 | 0.09 | 0.16 | 0.08 | 0.09 |
| NFKB1 | 1.00 ± | 1.08 ± | 0.87 ± | 1.03 ± | 1.00 ± | 0.92 ± | 1.04 ± | 0.98 ± | 1.05 ± | 1.14 ± |
| | 0.09 | 0.10 | 0.09 | 0.08 | 0.09 | 0.10 | 0.10 | 0.11 | 0.09 | 0.07 |
| PCNA | 1.00 ± | 1.47 ± | 0.78 ± | 0.74 ± | 1.36 ± | 0.72 ± | 1.26 ± | 0.85 ± | 1.38 ± | 0.77 ± |
| | 0.08 | 0.13 | 0.07 | 0.09 | 0.11 | 0.08 | 0.08 | 0.08 | 0.08 | 0.10 |
| PIDD | 1.00 ± | 3.79 ± | 1.61 ± | 1.25 ± | 3.47 ± | 1.44 ± | 3.84 ± | 2.42 ± | 3.53 ± | 1.71 ± |
| | 0.15 | 0.24 | 0.16 | 0.22 | 0.30 | 0.15 | 0.34 | 0.45 | 0.28 | 0.34 |
| PPM1D | 1.00 ± | 2.70 ± | 1.60 ± | 1.41 ± | 3.05 ± | 1.83 ± | 4.14 ± | 2.31 ± | 3.59 ± | 2.01 ± |
| | 0.05 | 0.17 | 0.16 | 0.13 | 0.31 | 0.29 | 0.24 | 0.15 | 0.17 | 0.19 |
| PRC1 | 1.00 ± | 0.36 ± | 0.37 ± | 0.17 ± | 0.11 ± | 0.10 ± | 0.09 ± | 0.13 ± | 0.13 ± | 0.14 ± |
| | 0.14 | 0.12 | 0.12 | 0.12 | 0.11 | 0.11 | 0.11 | 0.11 | 0.11 | 0.11 |
| PRKCA | 1.00 ± | 0.86 ± | 1.12 ± | 1.41 ± | 1.21 ± | 1.22 ± | 1.36 ± | 1.82 ± | 1.46 ± | 2.17 ± |
| | 0.16 | 0.13 | 0.14 | 0.16 | 0.14 | 0.18 | 0.14 | 0.28 | 0.18 | 0.19 |
| PTEN | 1.00 ± | 0.91 ± | 1.37 ± | 1.41 ± | 1.47 ± | 1.45 ± | 1.81 ± | 2.04 ± | 1.96 ± | 2.10 ± |
| | 0.21 | 0.18 | 0.28 | 0.19 | 0.18 | 0.26 | 0.21 | 0.27 | 0.27 | 0.21 |
| PTTG1 | 1.00 ± | 0.74 ± | 0.54 ± | 0.29 ± | 0.26 ± | 0.19 ± | 0.17 ± | 0.19 ± | 0.18 ± | 0.15 ± |
| | 0.16 | 0.13 | 0.15 | 0.13 | 0.13 | 0.13 | 0.13 | 0.13 | 0.13 | 0.13 |
| RB1 | 1.00 ± | 1.10 ± | 0.92 ± | 1.07 ± | 1.21 ± | 1.05 ± | 1.23 ± | 1.10 ± | 1.31 ± | 1.32 ± |
| | 0.07 | 0.11 | 0.08 | 0.08 | 0.07 | 0.08 | 0.11 | 0.10 | 0.07 | 0.13 |
| RELA | 1.00 ± | 0.93 ± | 1.04 ± | 1.16 ± | 1.04 ± | 1.18 ± | 1.34 ± | 1.29 ± | 1.38 ± | 1.53 ± |
| | 0.08 | 0.07 | 0.09 | 0.08 | 0.08 | 0.08 | 0.11 | 0.10 | 0.07 | 0.10 |
| RPRM | 1.00 ± | 0.53 ± | 0.82 ± | 1.30 ± | 1.36 ± | 1.38 ± | 1.42 ± | 1.80 ± | 1.36 ± | 1.55 ± |
| | 0.26 | 0.22 | 0.26 | 0.30 | 0.32 | 0.35 | 0.31 | 0.55 | 0.30 | 0.31 |
| SESN2 | 1.00 ± | 5.33 ± | 2.24 ± | 1.77 ± | 5.73 ± | 2.26 ± | 7.80 ± | 3.62 ± | 7.03 ± | 2.55 ± |
| | 0.20 | 0.74 | 0.30 | 0.21 | 0.47 | 0.31 | 0.90 | 0.40 | 0.59 | 0.34 |
| SIAH1 | 1.00 ± | 0.88 ± | 1.17 ± | 1.26 ± | 1.23 ± | 1.33 ± | 1.49 ± | 1.64 ± | 1.47 ± | 1.85 ± |
| | 0.05 | 0.07 | 0.12 | 0.07 | 0.06 | 0.11 | 0.09 | 0.18 | 0.12 | 0.14 |
| SIRT1 | 1.00 ± | 0.85 ± | 1.01 ± | 1.22 ± | 1.04 ± | 1.08 ± | 1.10 ± | 1.37 ± | 1.24 ± | 1.49 ± |
| | 0.05 | 0.09 | 0.05 | 0.06 | 0.05 | 0.05 | 0.07 | 0.10 | 0.08 | 0.09 |
| STAT1 | 1.00 ± | 0.82 ± | 0.89 ± | 1.00 ± | 0.80 ± | 1.01 ± | 1.16 ± | 1.03 ± | 1.13 ± | 1.08 ± |
| | 0.18 | 0.19 | 0.26 | 0.17 | 0.16 | 0.20 | 0.15 | 0.16 | 0.21 | 0.18 |
| TADA3 | 1.00 ± | 0.89 ± | 0.98 ± | 1.20 ± | 1.13 ± | 1.22 ± | 1.32 ± | 1.40 ± | 1.36 ± | 1.58 ± |
| | 0.07 | 0.06 | 0.06 | 0.06 | 0.06 | 0.06 | 0.10 | 0.10 | 0.08 | 0.07 |
| TNF | 1.00 ± | 1.41 ± | 0.92 ± | 0.90 ± | 0.85 ± | 0.63 ± | 0.75 ± | 0.49 ± | 0.69 ± | 0.45 ± |
| | 0.20 | 0.22 | 0.22 | 0.24 | 0.20 | 0.17 | 0.16 | 0.25 | 0.18 | 0.23 |
| TNFRSF10B | 1.00 ± | 2.78 ± | 1.83 ± | 1.18 ± | 2.78 ± | 1.36 ± | 2.88 ± | 1.84 ± | 2.72 ± | 1.11 ± |
| | 0.07 | 0.11 | 0.11 | 0.22 | 0.14 | 0.20 | 0.11 | 0.14 | 0.09 | 0.11 |
| TNFRSF10D | 1.00 ± | 2.15 ± | 1.51 ± | 1.31 ± | 2.53 ± | 1.35 ± | 2.67 ± | 2.00 ± | 3.15 ± | 1.76 ± |
| | 0.07 | 0.26 | 0.07 | 0.13 | 0.18 | 0.13 | 0.18 | 0.12 | 0.13 | 0.17 |
| TP53 | 1.00 ± | 0.72 ± | 0.72 ± | 0.88 ± | 0.72 ± | 0.84 ± | 0.87 ± | 0.82 ± | 0.83 ± | 0.99 ± |
| | 0.12 | 0.11 | 0.10 | 0.13 | 0.10 | 0.10 | 0.12 | 0.14 | 0.10 | 0.12 |
| TP53AIP1 | 1.00 ± | 1.02 ± | 1.21 ± | 1.71 ± | 1.99 ± | 1.45 ± | 1.72 ± | 2.72 ± | 2.10 ± | 2.92 ± |
| | 0.26 | 0.24 | 0.25 | 0.52 | 0.38 | 0.28 | 0.38 | 0.65 | 0.30 | 0.30 |
| TP53BP2 | 1.00 ± | 0.99 ± | 1.12 ± | 1.23 ± | 1.17 ± | 1.19 ± | 1.40 ± | 1.53 ± | 1.45 ± | 1.74 ± |
| | 0.05 | 0.07 | 0.09 | 0.07 | 0.05 | 0.09 | 0.07 | 0.10 | 0.07 | 0.09 |
| TP63 | 1.00 ± | 0.67 ± | 1.01 ± | 1.11 ± | 0.73 ± | 0.92 ± | 0.84 ± | 0.80 ± | 0.65 ± | 0.80 ± |
| | 0.20 | 0.20 | 0.18 | 0.19 | 0.17 | 0.17 | 0.20 | 0.19 | 0.20 | 0.19 |
| TP73 | 1.00 ± | 0.84 ± | 1.18 ± | 1.69 ± | 1.30 ± | 1.49 ± | 1.85 ± | 2.52 ± | 2.14 ± | 2.90 ± |
| | 0.16 | 0.15 | 0.20 | 0.39 | 0.17 | 0.23 | 0.23 | 0.53 | 0.38 | 0.31 |
| TRAF2 | 1.00 ± | 0.71 ± | 0.92 ± | 1.10 ± | 0.93 ± | 1.06 ± | 1.23 ± | 1.32 ± | 1.15 ± | 1.41 ± |
| | 0.06 | 0.06 | 0.07 | 0.16 | 0.05 | 0.06 | 0.07 | 0.12 | 0.07 | 0.15 |
| TSC1 | 1.00 ± | 0.88 ± | 1.18 ± | 1.41 ± | 1.33 ± | 1.41 ± | 1.62 ± | 1.93 ± | 1.58 ± | 1.95 ± |
| | 0.09 | 0.08 | 0.07 | 0.11 | 0.08 | 0.08 | 0.09 | 0.17 | 0.10 | 0.14 |
| WT1 | 1.00 ± | 1.03 ± | 1.40 ± | 2.24 ± | 1.35 ± | 1.92 ± | 2.17 ± | 3.19 ± | 2.97 ± | 4.05 ± |
| | 0.27 | 0.23 | 0.30 | 0.96 | 0.25 | 0.31 | 0.38 | 0.32 | 0.53 | 0.50 |
| XRCC5 | 1.00 ± | 0.87 ± | 0.92 ± | 1.12 ± | 0.94 ± | 1.04 ± | 1.15 ± | 1.14 ± | 1.17 ± | 1.36 ± |
| | 0.11 | 0.11 | 0.10 | 0.12 | 0.09 | 0.11 | 0.13 | 0.13 | 0.10 | 0.15 |
| ACTB | 1.00 ± | 0.95 ± | 1.01 ± | 0.96 ± | 0.85 ± | 0.85 ± | 0.90 ± | 0.89 ± | 0.90 ± | 0.94 ± |
| | 0.14 | 0.13 | 0.14 | 0.12 | 0.11 | 0.13 | 0.13 | 0.13 | 0.12 | 0.13 |
| B2M | 1.00 ± | 1.03 ± | 1.00 ± | 0.89 ± | 1.08 ± | 1.23 ± | 1.20 ± | 1.09 ± | 1.15 ± | 1.07 ± |
| | 0.18 | 0.21 | 0.16 | 0.14 | 0.15 | 0.16 | 0.18 | 0.18 | 0.17 | 0.19 |
| GAPDH | 1.00 ± | 1.07 ± | 1.15 ± | 1.25 ± | 0.98 ± | 1.00 ± | 1.08 ± | 1.07 ± | 1.10 ± | 1.08 ± |
| | 0.13 | 0.17 | 0.16 | 0.12 | 0.13 | 0.13 | 0.14 | 0.12 | 0.13 | 0.13 |
| HPRT1 | 1.00 ± | 0.88 ± | 0.77 ± | 0.84 ± | 0.82 ± | 0.74 ± | 0.69 ± | 0.80 ± | 0.72 ± | 0.79 ± |
| | 0.06 | 0.05 | 0.06 | 0.06 | 0.06 | 0.06 | 0.05 | 0.06 | 0.07 | 0.07 |
| RPLP0 | 1.00 ± | 1.11 ± | 1.11 ± | 1.10 ± | 1.32 ± | 1.29 ± | 1.23 ± | 1.20 ± | 1.21 ± | 1.15 ± |
| | 0.11 | 0.14 | 0.14 | 0.14 | 0.10 | 0.13 | 0.13 | 0.11 | 0.12 | 0.09 |

### Example 10: Efficacy and Tolerability of Compound 2 in a Mouse Xenograft Model of Gastric Cancer (NUGC3) when Treated with 25 mg/kg, 50 mg/kg, 100 mg/kg QD×21

The efficacy of Compound 2 was tested in a mouse xenograft model of gastric cancer (NUGC3). Nude mice bearing NUGC3 human gastric tumors were grown to ˜200 mm³ prior to being randomized into one of four study groups. Mice were dosed orally (PO) with either vehicle control (0.2% hydroxypropyl cellulose (HPC), 0.5% Tween 80) or Compound 2 at 25, 50, and 100 mg/kg daily for three weeks (QD×21). Following the final dose on day 20 all animals were bled for PK analysis at 8 h (N=4) and 24 h (N=4). Tumors from mice in groups 1, 5, and 6 were collected for PD analysis at the same time points. Tumors from mice in group 7 were too small for collection. TABLE 47 shows the dosing regimen for each treatment group of the study.

TABLE 47

| | | | | Dosing | | Dose |
|---|---|---|---|---|---|---|
| | | | | Frequency & | Dose | Volume |
| Group | Treatment | N | Route | Duration | (mg/kg) | (mL/kg) |
| 1 | Vehicle Control | 8 | PO | QDx21 | N/A | 10 |
| 5 | Compound 2 | 8 | PO | QDx21 | 25 | 10 |
| 6 | Compound 2 | 8 | PO | QDx21 | 50 | 10 |
| 7 | Compound 2 | 8 | PO | QDx21 | 100 | 10 |

Animals: Female Balb/c nude mice (150 total) were acclimatized and fed as described in EXAMPLE 7. Tumor cell culture; implantation of mice; and randomization and study group set up were carried out as described in EXAMPLE 7. Measurements and Calculation of Tumor Volume; and tumor lysate preparation were performed as described in EXAMPLE 5. TABLE 48 describes the tumor volumes and body weights of animals treated with vehicle control, 25 mg/kg, 50 mg/kg, and Compound 2 (QD×21).

TABLE 48

| | | Tumor Volume (mm³) | | | | Body Weight (g) | | | |
|---|---|---|---|---|---|---|---|---|---|
| Group | N | Mean | St Dev | Min | Max | Mean | St Dev | Min | Max |
| 1 - Vehicle Control | 8 | 229.88 | ±39.70 | 160.16 | 294.08 | 22.91 | ±1.42 | 20.20 | 24.60 |
| 5 - Compound 2 at | 8 | 229.74 | ±38.12 | 160.61 | 279.65 | 23.55 | ±1.09 | 22.20 | 25.00 |
| 25 mg/kg QDx21 | | | | | | | | | |
| 6 - Compound 2 at | 8 | 233.04 | ±34.73 | 177.87 | 279.35 | 22.94 | ±1.48 | 21.20 | 25.90 |
| 50 mg/kg QDx21 | | | | | | | | | |
| 7 - Compound 2 at | 8 | 233.08 | ±34.72 | 180.57 | 277.70 | 22.35 | ±1.18 | 20.60 | 24.1 |
| 100 mg/kg QDx21 | | | | | | | | | |

Mutant, WT, total p53, and p21 MSD: Mutant p53 (5 μg/mL), WT p53 (10 μg/mL), total p53 (5 μg/mL), and p21 Waf1/Cip1 (0.5 μg/mL) antibodies were coupled with U-Plex linkers by combining optimized concentrations for each antibody with the assigned linker. The samples were spun by vortex, and incubated for 30 minutes at RT before adding Stop Solution and incubating for another 30 minutes. The coupled antibody-linkers were combined into the same tube, and the total volume was adjusted with Stop Solution to 12 mL final volume. 96-well MSD U-Plex plates were coated with 50 μL/well of combined antibody-linker solution and incubated overnight at 4° C. on a shaker. Plates were washed 3× with wash buffer (1×TBS±0.1% Tween 20) and blocked with 1× blocking buffer (1×TBS±0.1% Tween 20±3% BSA). Tumor lysates were diluted in 1× lysis buffer to 0.4 μg/μL, blocking buffer was aspirated from the MSD plate, and 50 μL of tumor lysate was added to each well. The plate was sealed and incubated overnight at 4° C. on a shaker. Plates were washed 3× and treated with 50 μL/well detection antibody diluted in antibody diluent (1×TBS+0.1% Tween 20+1% BSA) (0.05 μg/mL; p53 7F5 Rabbit mAb, 0.05 μg/mL; p21 12D1 Rabbit mAb) for 1 h at RT. The plate was washed 3×, and the secondary antibody (Goat anti-Rabbit SULFO-TAG at 1 μg/mL) was added at 50 μL/well. The plates were then incubated for 1 h at RT on a shaker. The plate was finally washed 3×, 2×MSD Read Buffer was added at 150 μL/well, and the plate was read immediately on the MESO QuickPlex SQ 120. The MDM2 ELISA was run as described above in EXAMPLE 4.

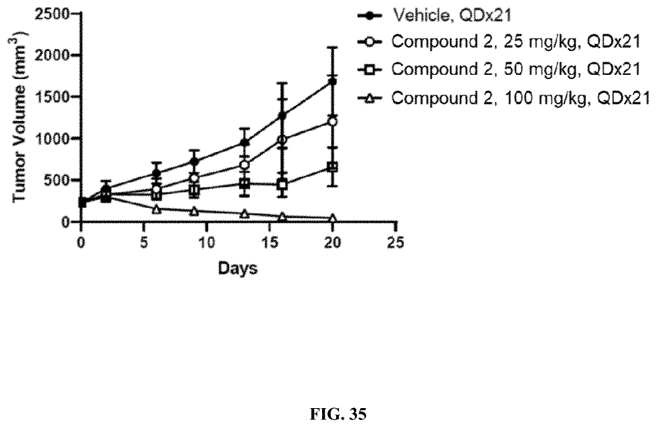

Efficacy: NUGC3 human gastric tumors grown in the female nude mice grew from an average of 229 mm³ to 1684 mm³ in 20 days. TABLE 49 shows the average percent tumor growth inhibition (%) of mice treated with the vehicle control, 50 mg/kg Compound 2 (QD×21), or 100 mg/kg Compound 2 (QD×21). TABLE 50 shows average percent tumor regression inhibition (%) of mice treated with the vehicle control, 50 mg/kg Compound 2 (QD×21), or 100 mg/kg Compound 2 (QD×21). Administration of Compound 2 at 25 and 50 mg/kg QD×21 resulted in 33.1% and 70.7% TGI, respectively, by day 20 of study, while administration of 100 mg/kg QD×21 resulted in 80.1% regression by day 20 of study. FIG. 35 shows changes in tumor volume (mm³) in mice treated with the vehicle control, 25 mg/kg Compound 2 (QD×21), 50 mg/kg Compound 2 (QD×21), and 100 mg/kg Compound 2 (QD×21).

TABLE 49

| Day of Study | 2 | 6 | 9 | 13 | 16 | 20 |
|---|---|---|---|---|---|---|
| Group 1 - | 0 | 0 | 0 | 0 | 0 | 0 |
| Vehicle Control | | | | | | |
| Group 5 - | 45.4 | 54.2 | 39.8 | 36.8 | 27.4 | 33.1 |
| Compound 2 | | | | | | |
| 25 mg/kg QDx21 | | | | | | |
| Group 6 - | 42.2 | 73.8 | 69.2 | 68.8 | 79.7 | 70.7 |
| Compound 2 | | | | | | |
| 50 mg/kg QDx21 | | | | | | |
| Group 7 - | 60.7 | >100 | >100 | >100 | >100 | >100 |
| Compound 2 | | | | | | |
| 100 mg/kg | | | | | | |
| QDx21 | | | | | | |

TABLE 50

| Day of Study | 2 | 6 | 9 | 13 | 16 | 20 |
|---|---|---|---|---|---|---|
| Group 1 - | 0 | 0 | 0 | 0 | 0 | 0 |
| Vehicle Control | | | | | | |
| Group 5 - | 0 | 0 | 0 | 0 | 0 | 0 |
| Compound 2 | | | | | | |
| 25 mg/kg QDx21 | | | | | | |
| Group 6 - | 0 | 0 | 0 | 0 | 0 | 0 |
| Compound 2 | | | | | | |
| 50 mg/kg QDx21 | | | | | | |
| Group 7 - | 0 | 33.3 | 43.4 | 56.5 | 71.7 | 80.1 |
| Compound 2 | | | | | | |
| 100 mg/kg QDx21 | | | | | | |

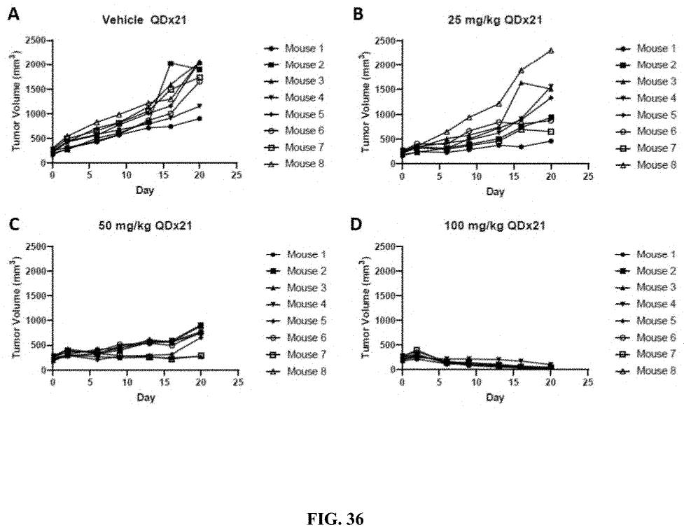

FIG. 36 PANEL A-PANEL D shows individual mouse tumor growth rates for mice treated with vehicle (QD×21), 25 mg/kg Compound 2 (QD×21), 50 mg/kg Compound 2 (QD×21), and 100 mg/kg Compound 2 (QD×21). NUGC3 tumors treated with vehicle control (0.2% HPC, 0.5% Tween 80) displayed consistent growth across the 20 days of study with the exception of mouse 2 where necrosis resulted in the tumor collapsing on the final measurement. All mice (N=8) administered Compound 2 at 100 mg/kg QD×21 resulted in a uniformed regression by day 6. Mice receiving Compound 2 at 50 mg/kg QD×21 showed consistent growth inhibition throughout the study while tumors on mice receiving 25 mg/kg QD×21 grew more variably beyond day 13 as tumor control was lost.

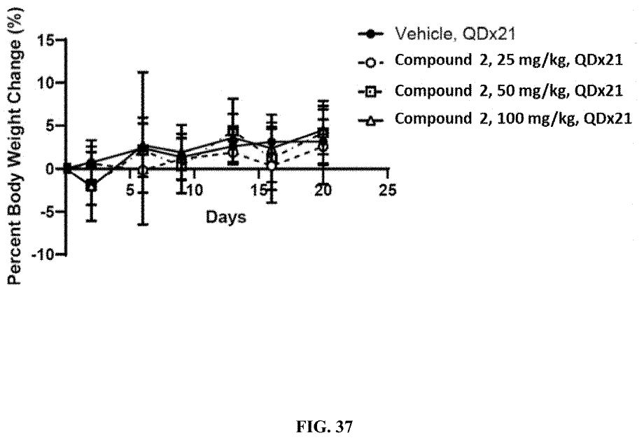

Body Weights: FIG. 37 shows average percent change in body weight (%, average±SD) for mice treated with the vehicle control (QD×21), 25 mg/kg Compound 2 (QD×21), 50 mg/kg Compound 2 (QD×21), and 100 mg/kg Compound 2 (QD×21). Average mouse body weights were well maintained over the course of the study. TABLE 51 shows average percent change in body weight across study (n=8 unless otherwise noted). Average mouse body weights were well maintained over the course of the study. Mice dosed with vehicle control remained on average around 23.3 g throughout the study with the percentage change varying between +0.65% early in the study to +3.11% by study completion. Mice administered with Compound 2 also maintained body weight throughout the study; mice dosed with 25 mg/kg QD×21 lost −0.21% body weight by day 6 and this recovered by day 9 of study to +1.17% while mice receiving 50 mg/kg QD×21 lost −2.07% body weight by day 2 that recovered to +2.07% by day 6. Group 7 mice administered 100 mg/kg QD×21 lost −2.13% body weight by day 2 of the study after which body weights increased to +2.63% by day 6. TABLE 52 summarizes clinical observations from the study.

TABLE 51

| | | Group 5 | Group 6 | Group 7 |
|---|---|---|---|---|
| | Group 1 | Compound 2 | Compound 2 | Compound 2 |
| Day of | Vehicle | 25 mg/kg | 50 mg/kg | 100 mg/kg |
| Study | Control | QDx21 | QDx21 | QDx21 |
| 2 | 0.73 ± 2.56 | 0.60 ± 1.97 | −2.02 ± 2.21 | −2.07 ± 4.00 |
| 6 | 2.37 ± 2.89 | −0.17 ± 2.64 | 2.13 ± 3.10 | 2.69 ± 3.23 |
| 9 | 1.37 ± 2.70 | 1.13 ± 2.43 | 0.32 ± 3.19 | 1.89 ± 3.20 |
| 13 | 2.57 ± 2.07 | 1.89 ± 1.41 | 4.40 ± 3.76 | 3.57 ± 2.78 |
| 16 | 3.10 ± 2.25 | 0.33 ± 4.30 | 1.17 ± 3.68 | 2.34 ± 3.92 |
| 20 | 3.14 ± 2.57 | 2.56 ± 4.36 | 4.17 ± 3.73 | 4.49 ± 2.81 |

TABLE 52

| | Mouse | Date of Death | |
|---|---|---|---|
| Group | Number | (Study Day) | Clinical Observations |
| Group 1 | 1 | 22 | None; euthanized for end of study 8 h post-dose |
| Vehicle | 2 | 22 | None; euthanized for end of study 8 h post-dose |
| Control | 3 | 22 | None; euthanized for end of study 8 h post-dose |
| | 4 | 22 | None; euthanized for end of study 8 h post-dose |
| | 5 | 22 | None; euthanized for end of study 24 h post- |
| | | | dose |
| | 6 | 22 | None; euthanized for end of study 24 h post- |
| | | | dose |
| | 7 | 22 | None; euthanized for end of study 24 h post- |
| | | | dose |
| | 8 | 22 | None; euthanized for end of study 24 h post- |
| | | | dose |
| Group 5 | 1 | 22 | None; euthanized for end of study 8 h post-dose |
| Compound 2 | 2 | 22 | None; euthanized for end of study 8 h post-dose |
| 25 mg/kg | 3 | 22 | None; euthanized for end of study 8 h post-dose |
| QDx21 | 4 | 22 | None; euthanized for end of study 8 h post-dose |
| | 5 | 22 | None; euthanized for end of study 24 h post- |
| | | | dose |
| | 6 | 22 | None; euthanized for end of study 24 h post- |
| | | | dose |
| | 7 | 22 | None; euthanized for end of study 24 h post- |
| | | | dose |
| | 8 | 22 | None; euthanized for end of study 24 h post- |
| | | | dose |
| Group 6 | 1 | 22 | None; euthanized for end of study 8 h post-dose |
| Compound 2 | 2 | 22 | None; euthanized for end of study 8 h post-dose |
| 50 mg/kg | 3 | 22 | None; euthanized for end of study 8 h post-dose |
| QDx21 | 4 | 22 | None; euthanized for end of study 8 h post-dose |
| | 5 | 22 | None; euthanized for end of study 24 h post- |
| | | | dose |
| | 6 | 22 | None; euthanized for end of study 24 h post- |
| | | | dose |
| | 7 | 22 | None; euthanized for end of study 24 h post- |
| | | | dose |
| | 8 | 22 | None; euthanized for end of study 24 h post- |
| | | | dose |
| Group 7 | 1 | 22 | None; euthanized for end of study 8 h post-dose |
| Compound 2 | 2 | 22 | None; euthanized for end of study 8 h post-dose |
| 100 mg/kg | 3 | 22 | None; euthanized for end of study 8 h post-dose |
| QDx21 | 4 | 22 | None; euthanized for end of study 8 h post-dose |
| | 5 | 22 | None; euthanized for end of study 24 h post- |
| | | | dose |
| | 6 | 22 | None; euthanized for end of study 24 h post- |
| | | | dose |
| | 7 | 22 | None; euthanized for end of study 24 h post- |
| | | | dose |
| | 8 | 22 | None; euthanized for end of study 24 h post- |
| | | | dose |

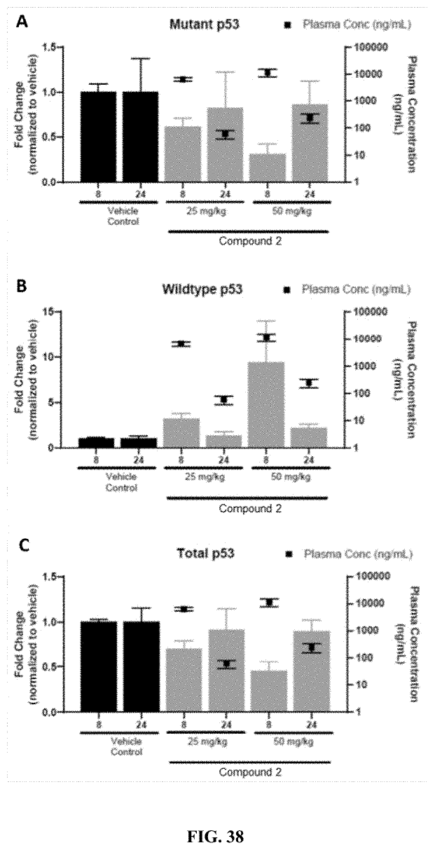

End of Efficacy PK/PD Results: Tumor and plasma from all mice were harvested for PD/PK analysis at 8 and 24 h post the final dose, with the exception of the 100 mg/kg group. The 100 mg/kg group had tumors that were too small for analysis, so only plasma samples were collected. All results shown are normalized to vehicle control. Tumors from mice administered with 25 mg/kg Compound 2 QD×21 showed a 3.2-fold increase in WT conformation p53 at 8 h, that returned to baseline by 24 h, a 39% and 18% decrease in mutant p53 and a 30% and 9% decrease in total levels of p53, at 8 and 24 h, respectively. Tumors from mice administered 50 mg/kg Compound 2 QD×21 and harvested 8 and 24 h post-dose showed a 9.4 and 2.2-fold increase in WT conformation p53, a 69% and 14% decrease in mutant p53 and a 55% and 11% decrease in total levels of p53, respectively. Peak plasma concentrations of Compound 2 were measured at 8 h post-dose and were 6310 ng/mL, 11,332 ng/mL, and 27,525 ng/mL at the 25 mg/kg, 50 mg/kg, and 100 mg/kg level, respectively. TABLE 53 shows the conversion of mutant p53 to wild type p53 configuration. FIG. 38 PANEL A-PANEL C show conversion of mutant p53 to wild-type p53 conformation in mice treated with vehicle control, Compound 2 25 mg/kg or 50 mg/kg.

TABLE 53

| | | | Fold Change over Vehicle Control or Percent Reduction | | | | | |
|---|---|---|---|---|---|---|---|---|
| | | | Relative to Vehicle Control (%) | | | | | |
| Group - | | Plasma | Mutant | WT | Total | | | |
| Compound 2 | Timepoint | Conc. | p53 | p53 | p53 | p21 | MDM2 | MIC-1 |
| (mg/kg) | (h) | (ng/mL) | Protein | Protein | Protein | Protein | Protein | Protein |
| Group 5 - | 8 | 6310 | 39% | 3.22 | 30% | 2.38 | 2.89 | 0.75* |
| 25 QDx21 | 24 | 60 | 18% | 1.31 | 9% | 0.89 | 1.09 | 0.06* |
| Group 6 - | 8 | 11332 | 69% | 9.38 | 55% | 4.21 | 15.75 | 4.77* |
| 50 QDx21 | 24 | 245 | 14% | 2.16 | 11% | 0.88 | 1.18 | 0.43* |
| Group 7 - | 8 | 27525 | N/A | N/A | N/A | N/A | N/A | 0.98* |
| 100 QDx21 | 24 | 33500 | N/A | N/A | N/A | N/A | N/A | 2.38* |

*= Absolute induction in pg/mL/mm³.

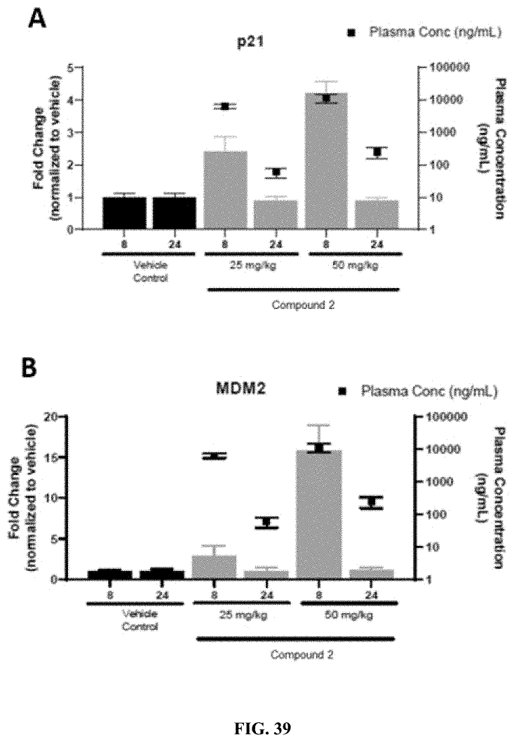

Measurement of p53 target proteins downstream of WT p53 showed a 2.4-fold increase in p21 and a 2.9-fold increase in MDM2 at 8 h post-dose in tumors from the 25 mg/kg QD×21 group; both targets returned to baseline by 24 h. Tumors from mice administered 50 mg/kg QD×21 demonstrated a 4.2-fold increase in p21 protein and a 15.8-fold increase in MDM2 at 8 h post-dose, returning to baseline by 24 h FIG. 39 PANEL A and PANEL B show fold changes normalized to vehicle and plasma concentration (ng/mL) of p21 and MDM2 for mice treated with the vehicle control, 25 mg/kg Compound 2, or 50 mg/kg Compound 2. The data show that conversion of mutant to WT conformation p53 resulted in downstream increases in the p53 target proteins p21 and MDM2.

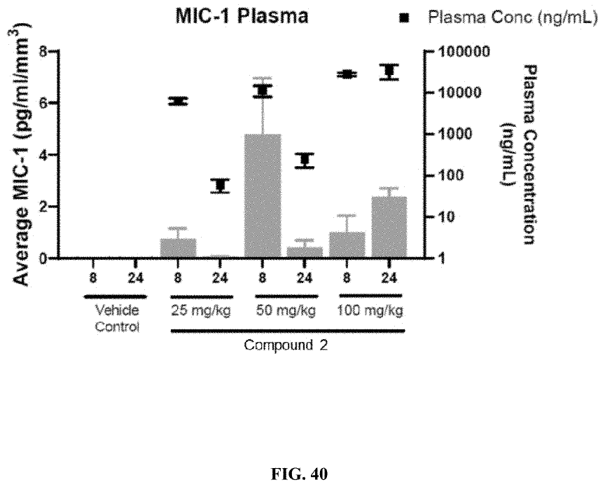

Increases in MIC-1 levels were measured in the plasma of the mice. Mice administered with 25 mg/kg Compound 2 QD×21 showed an induction of 0.75 μg/mL/mm³ at 8 h (normalized to tumor volume). This level returned to baseline levels by 24 h. Mice administered with 50 mg/kg Compound 2 QD×21 and harvested at 8 and 24 h post-dose resulted in induction of 4.77 and 0.43 μg/mL/mm³, respectively. Mice dosed with 100 mg/kg Compound 2 QD×21 demonstrated an induction of 0.98 and 2.38 μg/mL/mm³ at 8 and 24 h, respectively. FIG. 40 shows average plasma MIC-1 (pg/mL/mm³) and plasma concentration (ng/mL) for mice treated with the vehicle control, 25 mg/kg Compound 2, 50 mg/kg Compound 2, and 100 mg/kg Compound 2. The data show that conversion of mutant p53 to WT conformation p53 increased expression of MIC-1.

This study was designed to test the efficacy of Compound 2 in a mouse xenograft model of gastric cancer (NUGC3). Mice were dosed PO with either vehicle control (0.2% HPC, 0.5% Tween 80) or Compound 2 at 25, 50, and 100 mg/kg daily for three weeks (QD×21). By day 20 of study, tumors on mice receiving 25 and 50 mg/kg daily exhibited 33.1% and 70.7% tumor growth inhibition (TGI), respectively, while the 100 mg/kg daily regimen resulted in 80.1% regression.

The tumors showed dose responsive decreases in mutant p53 (39-69%) and increases in WT conformation p53 levels (3.2-9.4× vehicle control). Analysis of downstream p53 transcriptional targets p21 and MDM2 showed dose responsive increases in p21 (2.4-4.2×) and MDM2 (2.9-15.8×) proteins 8 h post-dose that returned to near-baseline levels by 24 h. Measurement of MIC-1 in the plasma showed non-dose responsive increases (0.8-4.8 pg/mL/mm³) at 8 h that returned to near-baseline by 24 h with the exception of the 100 mg/kg QD×21 group. The 100 mg/kg QD×21 group showed greater MIC-1 plasma exposure at 24 h (2.38 pg/mL/mm³), which corresponded with increased Compound 2 plasma exposure. Overall, daily administration of Compound 2 resulted in a dose responsive anti-tumor effect that correlated with a dose responsive target engagement and was well tolerated by the mice.

### Example 11: Efficacy and Tolerability of Compound 2 in a Mouse Xenograft Model of Pancreatic

Cancer (T3M-4) when administered orally 25 mg/kg (QD×18), 50 mg/kg (QD×18), 100 mg/kg (QD×18), 150 mg/kg (2Q7D×4), or 300 mg/kg (2Q7D×4).

The efficacy of Compound 2 was tested in a subcutaneous mouse xenograft model of pancreatic cancer (T3M-4). Mice bearing p53 Y220C mutant T3M-4 human pancreatic tumors were dosed orally (PO) once per day for 18 days (QD×18) with either vehicle control (0.2% HPC); 25 mg/kg Compound 2 (QD×18); 50 mg/kg Compound 2 (QD×18); 100 mg/kg Compound 2 (QD×18); 150 mg/kg Compound 2 twice daily, once per week for 4 weeks (2Q7D×4); or 300 mg/kg Compound 2 (2Q7D×4). All tumors and plasma were harvested for PD/PK analysis at 8 h and 24 h post the final dose (day 17 for groups 1-5 and day 21 for groups 6 and 7). TABLE 54 shows the treatment groups and dosing regimens for the study.

TABLE 54

| | | | | Dosing | | Dose |
|---|---|---|---|---|---|---|
| | | | | Frequency & | Dose | Volume |
| Group | Treatment | N | Route | Duration | (mg/kg) | (mL/kg) |
| 1 | Vehicle Control | 10 | PO | QDx18 | N/A | 10 |
| 2 | Compound 2 | 10 | PO | QDx18 | 25 | 10 |
| 3 | Compound 2 | 10 | PO | QDx18 | 50 | 10 |
| 4 | Compound 2 | 10 | PO | QDx18 | 100 | 10 |
| 5 | Compound 2 | 10 | PO | 2Q7Dx4 | 150 | 10 |
| 6 | Compound 2 | 10 | PO | 2Q7Dx4 | 300 | 10 |

Female Balb/c nude mice (150 total) were acclimatized as described in EXAMPLE 7. Implantation of mice and randomization and study set up procedures were also used as described in EXAMPLE 7. Average tumor volume (mm³) and body weight (g) is reported in TABLE 55.

Tumor Cell Culture: T3M-4 cells were cultured in DMEM-F12 medium with 10% fetal bovine serum. The cells were washed with PBS and counted at a total of 2.54×10⁹ cells with 94.5% viability. Cells were spun by centrifuge and resuspended in 75% PBS:25% Matrigel Matrix at a concentration of 1×10⁶ viable cells/100 μL.

TABLE 55

| | | Tumor Volume (mm³) | | | | Body Weight (g) | | | |
|---|---|---|---|---|---|---|---|---|---|
| Group | N | Mean | St Dev | Min | Max | Mean | St Dev | Min | Max |
| 1 - Vehicle Control | 10 | 171.20 | ±36.23 | 109.41 | 242.10 | 21.78 | 1.63 | 19.60 | 25.17 |
| 2 - Compound 2 at | 10 | 170.78 | ±34.81 | 114.76 | 239.40 | 22.33 | 1.06 | 20.50 | 23.85 |
| 25 mg/kg QDx18 | | | | | | | | | |
| 3 - Compound 2 at | 10 | 170.00 | ±35.11 | 116.13 | 233.15 | 22.85 | 1.82 | 20.00 | 25.37 |
| 50 mg/kg QDx18 | | | | | | | | | |
| 4 - Compound 2 at | 10 | 171.46 | ±36.49 | 115.25 | 237.22 | 23.44 | 2.38 | 19.60 | 28.50 |
| 100 mg/kg QDx18 | | | | | | | | | |
| 5 - Compound 2 at | 10 | 170.93 | ±36.36 | 118.41 | 229.53 | 22.51 | 1.19 | 21.10 | 24.50 |
| 150 mg/kg Q7Dx4 | | | | | | | | | |
| 6 - Compound 2 at | 10 | 168.87 | ±35.88 | 117.54 | 223.36 | 21.95 | 0.76 | 20.80 | 23.20 |
| 300 mg/kg Q7Dx4 | | | | | | | | | |

Measurements and Calculation of Tumor Volume; and tumor lysate preparation were performed as described in EXAMPLE 5. Mutant p53, WT conformation p53, total p53, and p21 MSD data were collected using the procedure described in EXAMPLE 10.

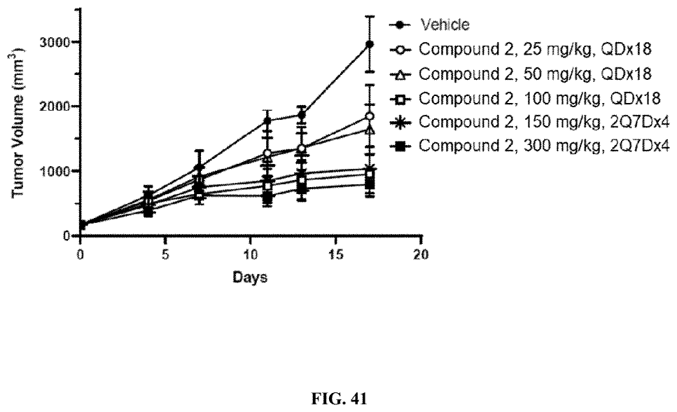

Efficacy: T3M-4 human pancreatic tumors grown in the female nude mice grew from an average of 171 mm³ to 2966 mm³ in 17 days. Daily PO administration of Compound 2 at 25 mg/kg, 50 mg/kg, and 100 mg/kg QD×18 resulted in 40%, 47%, and 72% Tumor Growth Inhibition (TGI), respectively, by day 17 of study. Weekly PO administration of 150 mg/kg and 300 mg/kg 2Q7D×4 resulted in 69% and 78% TGI, respectively, by day 17 of study. FIG. 41 shows changes in tumor volume (mm³) in mice treated with the vehicle control and Compound 2 at 25 mg/kg (QD×18), 50 mg/kg (QD×18), 100 mg/kg (QD×18), 150 mg/kg (2Q7D×4), or 300 mg/kg (2Q7D×4). TABLE 56 show tumor volumes across 17 days of the study.

TABLE 56

| Day of Study | 4 | 7 | 11 | 13 | 17 |
|---|---|---|---|---|---|
| Group 1 - Vehicle Control | 0 | 0 | 0 | 0 | 0 |
| Group 2 - Compound 2 | 18.2 | 21.2 | 31.4 | 30.6 | 39.9 |
| 25 mg/kgQDx18 | | | | | |
| Group 3 - Compound 2 | 14.8 | 15.8 | 35.0 | 30.1 | 47.2 |
| 50 mg/kgQDx18 | | | | | |
| Group 4 - Compound 2 | 27.9 | 46.1 | 62.5 | 59.1 | 72.0 |
| 100 mg/kgQDx18 | | | | | |
| Group 5 - Compound 2 | 32.2 | 34.3 | 57.7 | 53.0 | 69.0 |
| 150 mg/kgQ7Dx4 | | | | | |
| Group 6 - Compound 2 | 50.2 | 48.5 | 72.1 | 66.8 | 77.5 |
| 200 mg/kgQ7Dx4 | | | | | |

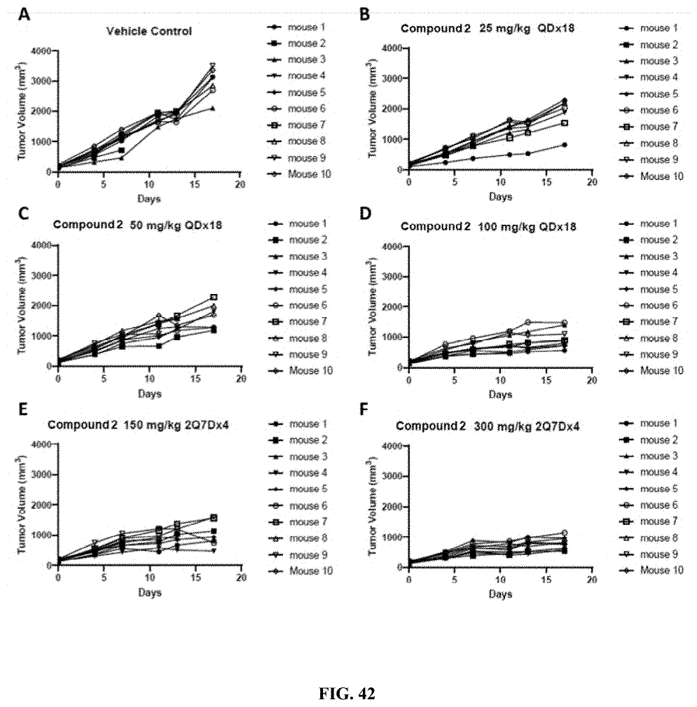

FIG. 42 PANEL A-PANEL F show changes in tumor volume for individual mice treated with vehicle control and Compound 2 at 25 mg/kg (QD×18), 50 mg/kg (QD×18), 100 mg/kg (QD×18), 150 mg/kg (2Q7D×4), or 300 mg/kg (2Q7D×4). TABLE 57 shows tumor volumes of individual mice treated with vehicle control and Compound 2 at 25 mg/kg (QD×18), 50 mg/kg (QD×18), 100 mg/kg (QD×18), 150 mg/kg (2Q7D×4), or 300 mg/kg (2Q7D×4). T3M-4 tumors treated with vehicle control (0.2% HPC) displayed consistent growth through 11 days of study. Mice 4 and 6 were slightly smaller due to tumor necrosis at day 13, thereafter returning to consistent growth rate. Administration of Compound 2 at 100 mg/kg resulted in consistent tumor growth control out through day 17 of study for all animals except for mouse 3 and 6. The mice in the 25 mg/kg and 50 mg/kg groups showed less control. Weekly administration of Compound 2 at 300 mg/kg 2Q7D×4 resulted in consistent tumor growth control of all animals through day 17 of study.

TABLE 57

| Day of Study | Mouse | 0 | 4 | 7 | 11 | 13 | 17 |
|---|---|---|---|---|---|---|---|
| Group 1 | 1 | 109 | 566 | 1028 | 1849 | 1959 | 3135 |
| Vehicle | 2 | 140 | 481 | 730 | — | — | — |
| Control | 3 | 145 | 336 | 472 | 1496 | 1754 | 2109 |
| | 4 | 163 | 706 | 1261 | 1960 | 1732 | 3109 |
| | 5 | 168 | 742 | 1088 | — | — | — |
| | 6 | 178 | 586 | 1170 | 1669 | 1646 | 2694 |
| | 7 | 177 | 646 | 1189 | 1941 | 2012 | — |
| | 8 | 193 | 634 | 1105 | 1654 | 1964 | 2848 |
| | 9 | 197 | 725 | 1133 | 1716 | 1894 | 3504 |
| | 10 | 242 | 855 | 1409 | 1958 | 1976 | 3361 |
| | Avg | 171 | 628 | 1059 | 1780 | 1867 | 2966 |
| | SD | 34 | 139 | 256 | 161 | 128 | 434 |
| Group 2 | 1 | 115 | 244 | 374 | 499 | 536 | 823 |
| 25 mg/kg | 2 | 140 | 554 | 776 | — | — | — |
| Compound2 | 3 | 143 | 481 | 788 | 1218 | 1337 | — |
| QDx18 | 4 | 161 | 553 | 939 | 1369 | 1416 | 1879 |
| | 5 | 169 | 523 | 933 | 1433 | 1644 | 2298 |
| | 6 | 173 | 723 | 1030 | 1635 | 1569 | 2025 |
| | 7 | 177 | 475 | 783 | 1045 | 1211 | 1543 |
| | 8 | 193 | 524 | 858 | 1411 | 1584 | 2190 |
| | 9 | 198 | 683 | 1099 | 1583 | 1490 | 2194 |
| | 10 | 239 | 681 | 1116 | — | — | — |
| | Avg | 171 | 544 | 870 | 1274 | 1348 | 1850 |
| | SD | 33 | 130 | 204 | 342 | 335 | 480 |
| Group 3 | 1 | 1 | 116 | 568 | 1017 | 1420 | 1560 |
| 50 mg/kg | 2 | 130 | 393 | 658 | 669 | 965 | 1185 |
| Compound 2 | 3 | 146 | 521 | 858 | — | — | — |
| QDx18 | 4 | 156 | 379 | 760 | 949 | 1224 | 1805 |
| | 5 | 167 | 489 | 859 | 1245 | 1307 | 1292 |
| | 6 | 176 | 499 | 866 | 1015 | 1175 | 1277 |
| | 7 | 178 | 606 | 958 | 1403 | 1675 | 2281 |
| | 8 | 195 | 715 | 1171 | 1490 | 1580 | 1995 |
| | 9 | 203 | 763 | 1049 | 1075 | — | — |
| | 10 | 233 | 655 | 974 | 1672 | 1358 | 1690 |
| | Avg | 170 | 559 | 917 | 1215 | 1355 | 1646 |
| | SD | 33 | 121 | 141 | 296 | 223 | 383 |
| Group 4 | 1 | 115 | 387 | 440 | 460 | 526 | 561 |
| 100 mg/kg | 2 | 130 | 369 | 442 | 498 | — | — |
| Compound 2 | 3 | 149 | 584 | 838 | 1067 | 1179 | 1409 |
| QDx18 | 4 | 157 | 456 | 617 | 682 | 818 | 885 |
| | 5 | 170 | 373 | 558 | 518 | 593 | 725 |
| | 6 | 174 | 770 | 965 | 1196 | 1492 | 1479 |
| | 7 | 179 | 471 | 587 | 758 | 820 | 901 |
| | 8 | 191 | 491 | 626 | 722 | 660 | 801 |
| | 9 | 212 | 640 | 789 | 1147 | 1053 | 1105 |
| | 10 | 237 | 464 | 633 | 707 | 647 | 727 |
| | Avg | 171 | 501 | 650 | 776 | 865 | 955 |
| | SD | 35 | 122 | 160 | 257 | 300 | 297 |
| Group 5 | 1 | 118 | 363 | 565 | 460 | 674 | 812 |
| 150 mg/kg | 2 | 127 | 377 | 670 | 785 | 1026 | 1132 |
| Compound 2 | 3 | 149 | 481 | 777 | — | — | — |
| 2Q7Dx4 | 4 | 151 | 321 | 440 | 586 | 510 | 478 |
| | 5 | 171 | 486 | 671 | 729 | 855 | 937 |
| | 6 | 173 | 525 | 887 | 974 | 1200 | 747 |
| | 7 | 179 | 556 | 907 | 1158 | 1378 | 1560 |
| | 8 | 192 | 447 | 755 | 921 | 904 | — |
| | 9 | 219 | 748 | 1056 | 1205 | 1202 | 1594 |
| | 10 | 230 | 498 | 811 | 848 | — | — |
| | Avg | 171 | 480 | 754 | 852 | 969 | 1037 |
| | SD | 35 | 115 | 169 | 232 | 272 | 387 |
| Group 6 | 1 | 118 | 289 | 533 | 503 | 529 | 633 |
| 300 mg/kg | 2 | 121 | 311 | 403 | 455 | 521 | 536 |
| Compound 2 | 3 | 149 | 410 | 662 | 786 | 691 | 822 |
| 2Q7Dx4 | 4 | 148 | 312 | 543 | 412 | 445 | 584 |
| | 5 | 171 | 526 | 883 | 824 | 987 | 958 |
| | 6 | 172 | 491 | 775 | 863 | 970 | 1138 |
| | 7 | 182 | 323 | 478 | 427 | — | — |
| | 8 | 189 | 460 | 715 | 690 | 837 | 941 |
| | 9 | 216 | 493 | 645 | 574 | 818 | 791 |
| | 10 | 223 | 348 | 622 | 638 | 793 | 764 |
| | Avg | 169 | 396 | 626 | 617 | 732 | 796 |
| | SD | 34 | 85 | 136 | 160 | 187 | 184 |

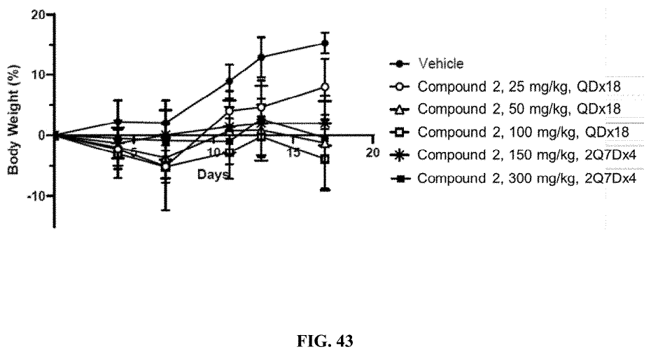

Body Weights: Mice administered Compound 2 experienced slight body weight loss proportional to total dose load across the study. The group dosed with vehicle control grew from 21.8 g to 24.9 g by day 17 with the percentage change increasing to +15.3% by day 17. Mice administered with Compound 2 at 25 mg/kg QD×18 experienced weight loss early, −2.3% by day 4, that continued and reached a maximum of −5.1% on day 7 before recovering to 8.0% on day 17. Mice that received 50 mg/kg Compound 2 QD×18 resulted in a maximum −3.8% weight loss on day 7 before recovering to −1.2% on day 17. Mice administered with 100 mg/kg Compound 2 QD×18 experienced maximum weight change of −5.2% on day 7, which recovered to −3.9% on day 17. Mice treated with Compound 2 at 150 mg/kg and 300 mg/kg 2Q7D×4 experienced immediate weight loss of −1.3% and −0.5%, respectively, on day 3, that recovered to 2.0% on day 17 for the 150 mg/kg group and −0.4% for the 300 mg/kg group. FIG. 43 shows change in body weight (%) proportional to total dose across the study for mice treated with the vehicle control and Compound 2 at 25 mg/kg (QD×18), 50 mg/kg (QD×18), 100 mg/kg (QD×18), 150 mg/kg (2Q7D×4), or 300 mg/kg (2Q7D×4). TABLE 58 shows change in body weight (%) proportional to total dose across the study. TABLE 59 shows the body weight (g) of individual mice in treatment groups across the duration of the study.

TABLE 58

| | | Group 2 | Group 3 | Group 4 | Group 5 | Group 6 |
|---|---|---|---|---|---|---|
| | Group 1 | Compound 2 | Compound 2 | Compound 2 | Compound 2 | Compound 2 |
| | Vehicle | 25 mg/kg | 50 mg/kg | 100 mg/kg | 150 mg/kg | 300 mg/kg |
| Day of Study | QDx18 | QDx18 | QDx18 | QDx18 | 2Q7Dx4 | 2Q7Dx4 |
| 4 | 2.25 ± 3.51 | −2.29 ± 4.76 | −2.02 ± 2.98 | −3.08 ± 2.46 | −1.30 ± 2.53 | −0.48 ± 2.94 |
| 7 | 2.08 ± 3.68 | −5.14 ± 7.31 | −3.75 ± 3.39 | −5.20 ± 2.66 | 0.04 ± 4.10 | −0.86 ± 2.70 |
| 11 | 8.92 ± 2.78 | 4.01 ± 3.34 | 0.74 ± 3.88 | −2.84 ± 4.36 | 1.45 ± 4.32 | −1.00 ± 3.82 |
| 13 | 12.89 ± 3.33 | 4 60 ± 4.47 | 0.86 ± 4.21 | −0.21 ± 3.36 | 2.00 ± 6.20 | 2.67 ± 3.36 |
| 17 | 15.25 ± 1.73 | 8.02 ± 4.63 | −1.24 ± 7.82 | −3.93 ± 4.93 | 1.99 ± 3.65 | −0.43 ± 3.09 |

TABLE 59

| | | Day of Study | | | | | |
|---|---|---|---|---|---|---|---|
| | Mouse | 0 | 4 | 7 | 11 | 13 | 17 |
| Group 1 | 1 | 21.2 | 22.9 | 21.7 | 23.1 | 23.8 | 24.2 |
| Vehicle | 2 | 22.4 | 23.6 | 23.7 | — | — | — |
| Control | 3 | 20.4 | 20.2 | 20.5 | 22.5 | 22.7 | 23.8 |
| | 4 | 22.7 | 23.5 | 23.4 | 24.3 | 24.9 | 26.6 |
| | 5 | 21.0 | 21.2 | 20.5 | — | — | — |
| | 6 | 20.3 | 20.6 | 20.2 | 22.6 | 23.3 | 23.7 |
| | 7 | 23.0 | 24.3 | 25.0 | 26.0 | 26.7 | — |
| | 8 | 19.6 | 19.7 | 20.5 | 21.4 | 21.9 | 22.2 |
| | 9 | 25.2 | 25.9 | 25.5 | 27.0 | 27.7 | 29.2 |
| | 10 | 22.0 | 21.1 | 21.4 | 22.9 | 23.9 | 24.9 |
| | Avg | 21.8 | 22.3 | 22.2 | 23.7 | 24.4 | 24.9 |
| | SD | 1.6 | 2.0 | 2.0 | 1.9 | 2.0 | 2.3 |
| Group 2 | 1 | 23.9 | 23.6 | 22.9 | 24.6 | 23.9 | 24.9 |
| 25 mg/kg | 2 | 22.0 | 20.1 | 20.1 | — | — | — |
| Compound 2 | 3 | 22.5 | 22.5 | 22.9 | 23.0 | 23.4 | — |
| QDx18 | 4 | 23.1 | 22.2 | 22.2 | 23.4 | 23.7 | 24.1 |
| | 5 | 23.2 | 23.8 | 21.1 | 24.5 | 24.2 | 25.1 |
| | 6 | 20.9 | 21.1 | 21.2 | 22.3 | 23.3 | 24.0 |
| | 7 | 23.0 | 22.4 | 22.3 | 23.1 | 23.2 | 23.8 |
| | 8 | 22.5 | 23.3 | 22.8 | 24.8 | 25.1 | 25.5 |
| | 9 | 20.5 | 20.1 | 19.6 | 21.0 | 20.8 | 22.1 |
| | 10 | 21.8 | 19.2 | 16.8 | — | — | — |
| | Avg | 22.3 | 21.8 | 21.2 | 23.3 | 23.5 | 24.2 |
| | SD | 1.1 | 1.6 | 1.9 | 1.3 | 1.2 | 1.1 |
| Group 3 | 1 | 24.5 | 23.5 | 23.6 | 26.0 | 25.6 | — |
| 50 mg/kg | 2 | 20.0 | 20.1 | 19.7 | 20.7 | 20.6 | 21.3 |
| Compound 2 | 3 | 22.2 | 22.1 | 21.0 | — | — | — |
| QDx18 | 4 | 25.4 | 24.6 | 24.2 | 25.3 | 26.1 | 25.8 |
| | 5 | 22.2 | 20.5 | 19.6 | 20.4 | 20.2 | 18.9 |
| | 6 | 20.7 | 21.1 | 20.9 | 21.2 | 20.8 | 18.7 |
| | 7 | 23.8 | 23.4 | 23.1 | 23.8 | 24.4 | 24.4 |
| | 8 | 24.6 | 23.5 | 23.8 | 25.1 | 25.2 | 25.3 |
| | 9 | 21.4 | 21.6 | 21.3 | 21.5 | — | — |
| | 10 | 23.8 | 23.1 | 22.6 | 23.8 | 23.7 | 24.3 |
| | Avg | 22.8 | 22.4 | 22.0 | 23.1 | 23.3 | 22.7 |
| | SD | 1.8 | 1.5 | 1.7 | 2.2 | 2.4 | 3.0 |
| Group 4 | 1 | 25.0 | 23.7 | 22.9 | 23.3 | 24.4 | 24.5 |
| 100 mg/kg | 2 | 23.9 | 23.7 | 22.3 | 21.4 | — | — |
| Compound 2 | 3 | 21.5 | 21.5 | 21.4 | 21.6 | 22.3 | 19.3 |
| QDx18 | 4 | 24.4 | 23.2 | 22.7 | 23.7 | 24.1 | 23.9 |
| | 5 | 21.8 | 20.5 | 20.4 | 20.8 | 21.6 | 21.1 |
| | 6 | 23.8 | 24.0 | 23.2 | 24.8 | 24.6 | 24.6 |
| | 7 | 19.6 | 18.6 | 19.0 | 19.3 | 19.5 | 18.1 |
| | 8 | 22.9 | 22.5 | 21.9 | 22.0 | 22.5 | 21.0 |
| | 9 | 28.5 | 27.1 | 26.1 | 26.9 | 26.8 | 26.3 |
| | 10 | 23.1 | 22.5 | 22.2 | 23.7 | 24.0 | 23.8 |
| | Avg | 23.4 | 22.7 | 22.2 | 22.8 | 23.3 | 22.5 |
| | SD | 2.4 | 2.3 | 1.9 | 2.2 | 2.1 | 2.7 |
| Group 5 | 1 | 21.2 | 21.0 | 21.4 | 21.5 | 22.4 | 22.2 |
| 150 mg/kg | 2 | 22.5 | 21.6 | 22.0 | 22.1 | 22.6 | 23.0 |
| Compound 2 | 3 | 24.5 | 24.4 | 23.8 | — | — | — |
| 2Q7Dx4 | 4 | 22.5 | 22.7 | 22.9 | 23.3 | 24.6 | 24.5 |
| | 5 | 23.3 | 22.8 | 23.5 | 23.7 | 24.4 | 23.6 |
| | 6 | 21.1 | 21.8 | 23.0 | 23.5 | 23.3 | 20.7 |
| | 7 | 24.2 | 22.8 | 22.5 | 23.5 | 24.1 | 24.0 |
| | 8 | 21.6 | 21.5 | 21.8 | 22.1 | 20.7 | — |
| | 9 | 21.6 | 21.1 | 21.7 | 21.3 | 21.9 | 21.7 |
| | 10 | 22.5 | 22.4 | 22.3 | 22.5 | — | — |
| | Avg | 22.5 | 22.2 | 22.5 | 22.6 | 23.0 | 22.8 |
| | SD | 1.2 | 1.0 | 0.8 | 0.9 | 1.3 | 1.3 |
| Group 6 | 1 | 20.8 | 21.5 | 21.5 | 21.6 | 22.3 | 21.1 |
| 300 mg/kg | 2 | 22.1 | 22.1 | 21.8 | 21.8 | 22.3 | 22.0 |
| Compound 2 | 3 | 21.1 | 21.8 | 21.5 | 21.9 | 22.0 | 21.3 |
| 2Q7Dx4 | 4 | 21.4 | 21.2 | 21.1 | 20.9 | 22.4 | 21.3 |
| | 5 | 23.2 | 23.5 | 23.0 | 23.4 | 23.6 | 22.7 |
| | 6 | 22.0 | 21.8 | 21.6 | 21.3 | 22.0 | 21.0 |
| | 7 | 22.8 | 21.4 | 22.3 | 21.0 | — | — |
| | 8 | 22.1 | 22.1 | 22.0 | 22.3 | 23.1 | 22.8 |
| | 9 | 21.5 | 21.5 | 21.7 | 21.8 | 22.6 | 22.2 |
| | 10 | 22.5 | 21.5 | 21.0 | 21.3 | 21.6 | 21.3 |
| | Avg | 21.9 | 21.8 | 21.7 | 21.7 | 22.4 | 21.8 |
| | SD | 0.8 | 0.7 | 0.6 | 0.7 | 0.6 | 0.7 |

Clinical Observations: Despite minimal weight loss, Compound 2 was tolerated throughout the study with no significant clinical observations across treatment groups. Mouse 10 in the 25 mg/kg QD×18 group experienced −11.7% and −22.9% body weight loss on days 4 and 7, respectively. The result was likely caused by a dosing accident, and the mouse was euthanized as a humane endpoint on day 7. All groups experienced one to four tumors that became openly necrotic between days 7 and 13 and required euthanasia prior to study termination. TABLE 60 shows clinical observations of individual mice in each treatment group.

TABLE 60

| | Mouse | Date of Death | |
|---|---|---|---|
| Group | Number | (Study Day) | Clinical Observations |
| Group 1 | 1 | 17 | None; euthanized for end of study 8 h post dose |
| Vehicle | 2 | 7 | Necrotic tumor requiring euthanasia |
| Control | 3 | 17 | None; euthanized for end of study 8 h post dose |
| | 4 | 17 | Tumor noted necrotic; euthanized for end of study 8 h |
| | | | post dose |
| | 5 | 7 | Necrotic tumor requiring euthanasia |
| | 6 | 18 | Tumor noted necrotic; euthanized for end of study 24 |
| | | | h post dose |
| | 7 | 13 | Necrotic tumor requiring euthanasia |
| | 8 | 18 | None; euthanized for end of study 24 h post dose |
| | 9 | 18 | None; euthanized for end of study 24 h post dose |
| | 10 | 18 | None; euthanized for end of study 24 h post dose |
| Group 2 | 1 | 17 | None; euthanized for end of study 8 h post dose |
| Compound 2 | 2 | 7 | Necrotic tumor requiring euthanasia |
| 25 mg/kg | 3 | 13 | Necrotic tumor requiring euthanasia |
| QDx18 | 4 | 17 | None; euthanized for end of study 8 h post dose |
| | 5 | 17 | None; euthanized for end of study 8 h post dose |
| | 6 | 18 | None; euthanized for end of study 24 h post dose |
| | 7 | 18 | None; euthanized for end of study 24 h post dose |
| | 8 | 18 | None; euthanized for end of study 24 h post dose |
| | 9 | 18 | None; euthanized for end of study 24 h post dose |
| | 10 | 7 | Body weight loss 22.9% requiring euthanasia |
| Group 3 | 1 | 13 | Necrotic tumor requiring euthanasia |
| Compound 2 | 2 | 17 | None; euthanized for end of study 8 h post dose |
| 50 mg/kg | 3 | 7 | Necrotic tumor requiring euthanasia |
| QDx18 | 4 | 17 | None; euthanized for end of study 8 h post dose |
| | 5 | 17 | None; euthanized for end of study 8 h post dose |
| | 6 | 7 | Necrotic tumor requiring euthanasia |
| | 7 | 18 | None; euthanized for end of study 24 h post dose |
| | 8 | 18 | None; euthanized for end of study 24 h post dose |
| | 9 | 11 | Necrotic tumor requiring euthanasia |
| | 10 | 18 | None; euthanized for end of study 24 h post dose |
| Group 4 | 1 | 17 | None; euthanized for end of study 8 h post dose |
| Compound 2 | 2 | 11 | Necrotic tumor requiring euthanasia |
| 100 mg/kg | 3 | 17 | Tumor too necrotic for collection, euthanized |
| QDx18 | 4 | 17 | None; euthanized for end of study 8 h post dose |
| | 5 | 17 | None; euthanized for end of study 8 h post dose |
| | 6 | 18 | None; euthanized for end of study 24 h post dose |
| | 7 | 18 | None; euthanized for end of study 24 h post dose |
| | 8 | 18 | None; euthanized for end of study 24 h post dose |
| | 9 | 18 | None; euthanized for end of study 24 h post dose |
| | 10 | 18 | None; euthanized for end of study 24 h post dose |
| Group 5 | 1 | 21 | None; euthanized for end of study 8 h post dose |
| Compound 2 | 2 | 21 | None; euthanized for end of study 8 h post dose |
| 150 mg/kg | 3 | 7 | Necrotic tumor requiring euthanasia |
| 2Q7Dx4 | 4 | 21 | None; euthanized for end of study 8 h post dose |
| | 5 | 22 | None; euthanized for end of study 24 h post dose |
| | 6 | 17 | Necrotic tumor requiring euthanasia |
| | 7 | 22 | None; euthanized for end of study 24 h post dose |
| | 8 | 13 | Necrotic tumor requiring euthanasia |
| | 9 | 22 | None; euthanized for end of study 24 h post dose |
| | 10 | 11 | Necrotic tumor requiring euthanasia |
| Group 6 | 1 | 21 | None; euthanized for end of study 8 h post dose |
| Compound 2 | 2 | 21 | None; euthanized for end of study 8 h post dose |
| 300 mg/kg | 3 | 21 | None; euthanized for end of study 8 h post dose |
| 2Q7Dx4 | 4 | 21 | None; euthanized for end of study 8 h post dose |
| | 5 | 21 | None; euthanized for end of study 8 h post dose |
| | 6 | 22 | None; euthanized for end of study 24 h post dose |
| | 7 | 11 | Necrotic tumor requiring euthanasia |
| | 8 | 22 | None; euthanized for end of study 24 h post dose |
| | 9 | 22 | None; euthanized for end of study 24 h post dose |
| | 10 | 22 | None; euthanized for end of study 24 h post dose |

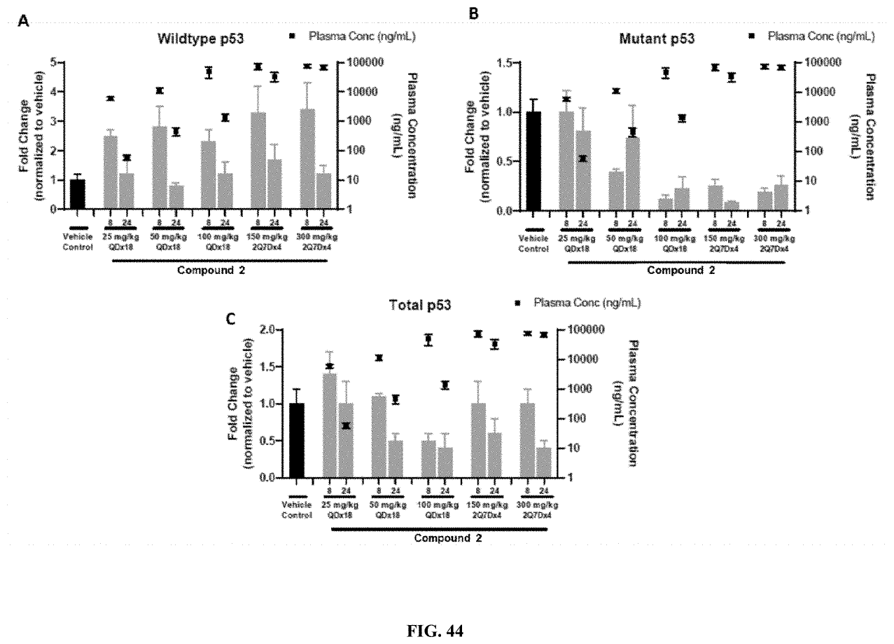

End of Efficacy PK/PD Results: Mice in groups 1-6 were harvested for PD/PK analysis at 8 h and 24 h post their final dose; day 17 for vehicle and daily Compound 2 treatment groups (1-4); and day 21 for the weekly Compound 2 treatment groups (groups 5-6). Tumors from mice treated with 25 mg/kg Compound 2 QD×18 showed a 2.50- and 1.20-fold increase in WT conformation p53 at 8 h and 24 h, respectively; and a 19.2% and 0.0% decrease in mutant and total p53, respectively, at 24 h. Mice administered 50 mg/kg QD×18 resulted in a 2.8-fold increase in WT conformation p53 at 8 h; and a 61.1% decrease in mutant p53 at 8 h; while reductions of 20.0%, 26.2%, and 50.0% in WT, mutant and total p53 were observed at 24 h, respectively. Daily administration of 100 mg/kg QD×18 resulted in increases in WT conformation p53 of 2.3- and 1.2-fold at 8 h and 24 h, respectively; decreases in mutant p53 of 88.2% and 77.8% at 8 h and 24 h, respectively; and reductions in total p53 levels of 50.0% at 8 h and 60% at 24 h when compared to control tumors. Tumors from mice administered 150 mg/kg 2Q7D×4 resulted in a 3.3- and 1.7-fold increase in WT conformation p53 at 8 and 24 h, respectively; a 74.6 and 91.7% decrease in mutant p53 at 8 and 24 h, respectively; and a 40.0% decrease in total p53 at 24 h. Administration of Compound 2 at 300 mg/kg 2Q7D×4 resulted in a 3.4- and 1.2-fold increase in WT conformation p53 at 8 and 24 h, respectively; a 81.1 and 73.9% decrease in mutant p53 at 8 and 24 h, respectively; and a 60.0% reduction in total p53 by 24 h. Plasma concentrations were approximately at the expected levels for the given doses. FIG. 44 PANEL A-PANEL C show fold change normalized to vehicle and plasma concentration (ng/mL) of mice treated with the vehicle control and Compound 2 at 25 mg/kg (QD×18), 50 mg/kg (QD×18), 100 mg/kg (QD×18), 150 mg/kg (2Q7D×4), or 300 mg/kg (2Q7D×4). TABLE 61 shows conversion of mutant p53 to wild-type p53 conformation.

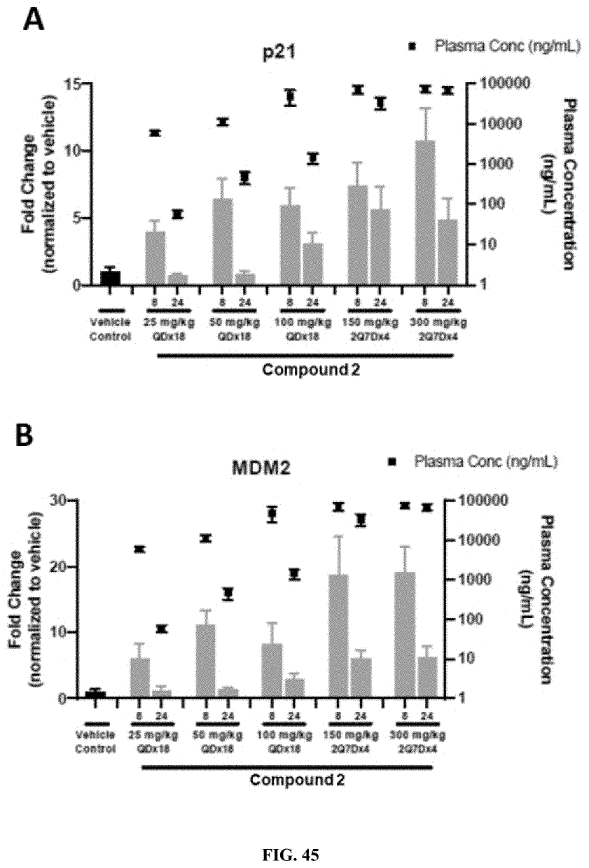

Measurement of p53 target proteins p21 and MDM2 showed increases of 3.9- and 6.4-fold in p21 for mice administered 25 mg/kg and 50 mg/kg Compound 2 QD×18, respectively; and increases of 6.1- and 11.1-fold in MDM2 for mice administered 25 mg/kg and 50 mg/kg Compound 2 QD×18, respectively at 8 h post dose. The levels of p21 and MDM2 returned to baseline levels at 24 h. Administration of 100 mg/kg QD×18 resulted in increases of 5.9- and 3.0-fold in p21 at 8 and 24 h, respectively; and increases of 8.2- and 2.9-fold in MDM2 at 8 and 24 h, respectively. Tumors from mice dosed with 150 mg/kg 2Q7D×4 resulted in 7.4- and 5.6-fold increases of p21 levels at 8 and 24 h, respectively; and 18.6- and 6.1-fold increases in MDM2 at 8 and 24 h, respectively. Administration of 300 mg/kg 2Q7D×4 showed a 10.7- and 4.8-fold increase in p21 at 8 and 24 h, respectively; and increases in MDM2 levels of 19.1- and 6.3-fold at 8 and 24 h, respectively. FIG. 45 PANEL A and PANEL B show fold changes normalized to vehicle and plasma concentration (ng/mL) of p21 and MDM2 in mice treated with the vehicle control and Compound 2 at 25 mg/kg (QD×18), 50 mg/kg (QD×18), 100 mg/kg (QD×18), 150 mg/kg (2Q7D×4), or 300 mg/kg (2Q7D×4). TABLE 61 shows conversion of mutant to WT conformation p53 results in downstream increases in p53 target proteins: p21 and MDM2.

TABLE 61

| | | | Fold Change over Vehicle Control or Percent Reduction | | | | |
|---|---|---|---|---|---|---|---|
| | | | Relative to Vehicle Control (%) | | | | |
| Group - | | Plasma | Mutant | WT | Total | | |
| Compound 2 | Timepoint | Conc. | p53 | p53 | p53 | p21 | MDM2 |
| (mg/kg) | (h) | (ng/mL) | Protein | Protein | Protein | Protein | Protein |
| Group 2 - | 8 | 5897 | 1.01 | 2.50 | 1.40 | 3.90 | 6.10 |
| 25 QDx18 | 24 | 58 | 19.2% | 1.20 | 0.0% | 30.0% | 1.20 |
| Group 3 - | 8 | 11390 | 61.1% | 2.80 | 1.10 | 6.40 | 11.10 |
| 50 QDx18 | 24 | 461 | 26.2% | 20.0% | 50.0% | 20.0% | 1.30 |
| Group 4 - | 8 | 48833 | 88.2% | 2.30 | 50.0% | 5.90 | 8.20 |
| 100 QDx18 | 24 | 1382 | 77.8% | 1.20 | 60.0% | 3.00 | 2.90 |
| Group 5 - | 8 | 73333 | 74.6% | 3.30 | 0.0% | 7.40 | 18.60 |
| 150 2Q7Dx4 | 24 | 34000 | 91.7% | 1.70 | 40.0% | 5.60 | 6.10 |
| Group 6 - | 8 | 75800 | 81.1% | 3.40 | 0.0% | 10.70 | 19.10 |
| 300 2Q7Dx4 | 24 | 69625 | 73.9% | 1.20 | 60.0% | 4.80 | 6.30 |

The anti-tumor effect of Compound 2 was tested at multiple doses and schedules in a mouse xenograft model of pancreatic cancer (T3M-4). Daily PO administration of Compound 2 at 25 mg/kg, 50 mg/kg, and 100 mg/kg QD×18 resulted in TGI values of 40%, 47%, and 72.0%, respectively by day 17 of study. Weekly PO administration of Compound 2 at 150 mg/kg and 300 mg/kg 2Q7D×4 resulted in 69% and 78% TGI by day 17, respectively. Treatment with all regimens of Compound 2 resulted in a dose-dependent minor weight loss of <5%.

At termination of the study, tumor and plasma were collected for PD/PK analysis. Plasma concentrations for all doses were in the expected range for the dose level and timepoint. Compound 2 treatment resulted in increases of WT conformation p53 (2.3- to 3.4-fold) at 8 h which was reduced by 24 h to 1.2- to 1.7-fold over vehicle control-treated tumors. Reduction in mutant p53 was robust at 8 h (61-88%) for all regimens at 8 h except the 25 mg/kg QD×18 group. An increased PD response was not observed in the 300 mg/kg 2Q7D×4 group over the 150 mg/kg 2Q7D×4 group, consistent with similar efficacy and PK exposure between the two groups. Increases in the WT conformation p53 target proteins p21 and MDM2 were also measured 8 h post dose.

### Example 12: Measurement of the Pharmacodynamic and PK Response to Compound 2 in a Mouse Xenograft Model of Pancreatic Cancer (T3M-4) when Administered 50 mg/kg (QD×6) or 100 mg/kg (QD×6)

The PD and PK relationship of Compound 2 was studied following daily dosing in a mouse xenograft model of pancreatic cancer (T3M-4). Mice bearing p53 Y220C mutant T3M-4 human pancreatic tumors were administered either vehicle or Compound 2. Group 1 animals were dosed orally (PO) for 6 days (QD×6) with vehicle control (0.2% HPC, 0.5% Tween 80). Tumor and plasma were harvested at 8 and 24 h post the first single dose and also 8 h post dose on day 2, 4, and 6. Group 2 and 3 mice were dosed with Compound 2 at 50 mg/kg and 100 mg/kg QD×6, respectively, and harvested at 4, 8, 16, and 24 h post the first single dose and 8 h and 24 h post dose on day 2, 4, and 6. Plasma was analyzed for Compound 2 levels by LC/MS-MS while tumors were analyzed for mutant p53, WT p53, and total p53 protein levels. Downstream induction of p53 target gene transcription and protein levels were studied as evidence of target engagement. TABLE 62 shows the treatment groups and dosing regimens.

TABLE 62

| | | | | Dosing | | |
|---|---|---|---|---|---|---|
| | | | | Frequency & | Dose | Harvest timepoints |
| Group | Treatment | N | Route | Duration | (mg/kg) | (h post last dose) |
| 1 | Vehicle Control | 15 | PO | QDx6 | N/A | 8, 24, 32, 80, 128 |
| | | (3/tp) | | | | |
| 2 | Compound 2 | 40 | PO | QDx6 | 50 | 4, 8, 16, 24, 32, 48, |
| | | (4/tp) | | | | 80, 96, 128, 144 |
| 3 | Compound 2 | 40 | PO | QDx6 | 100 | 4, 8, 16, 24, 32, 48, |
| | | (4/tp) | | | | 80, 96, 128, 144 |

Female Balb/c nude mice (250 total) were acclimatized as described in EXAMPLE 7. T3M-4 cells were cultured as described in EXAMPLE 11. Implantation of mice and randomization and study set up procedures were also used as described in EXAMPLE 7. Treatment began on the 14^{th} day to facilitate the collection schedule. TABLE 63 shows average tumor volume (mm³) and body weight (g) of mice according to treatment groups.

TABLE 63

| | | Tumor Volume (mm³) | | | | Body Weight (g) | | | |
|---|---|---|---|---|---|---|---|---|---|
| Group | N | Mean | St Dev | Min | Max | Mean | St Dev | Min | Max |
| 1 - Vehicle Control | 15 | 510.47 | ±133.80 | 370.95 | 823.95 | 22.94 | ±1.90 | 19.72 | 26.84 |
| 2 - Compound 2 at | 40 | 340.32 | ±108.78 | 106.95 | 707.17 | 23.47 | ±1.51 | 20.27 | 28.00 |
| 50 mg/kg QDx6 | | | | | | | | | |
| 3 - Compound 2 at | 40 | 354.42 | ±112.28 | 167.29 | 689.15 | 22.90 | ±1.64 | 19.74 | 26.71 |
| 100 mg/kg QDx6 | | | | | | | | | |

Measurements and Calculation of Tumor Volume; and tumor lysate preparation were performed as described in EXAMPLE 5. Mutant p53, WT p53, total p53, and p21 MSD data were collected using the procedure described in EXAMPLE 10. The plasma MIC-1 ELISA was performed as described in EXAMPLE 9. p21, MDM2, BIRC5, and GAPDH gene expression experiments were performed as described in EXAMPLE 9. Human p53 signaling pathway profiling experiment were performed as described in EXAMPLE 3.

PK/PD of Compound 2: Administration of Compound 2 at 50 mg/kg QD×1 resulted in a 26% and 39% reduction in mutant p53 16 and 24 h post dose, respectively, when compared to averaged vehicle control samples. Continued daily administration resulted in reductions of mutant p53 of 75%, 47%, and 47% 8 h post dose on days 2, 4, and 6, respectively, correlating with peak plasma concentrations of approximately 11,000 ng/mL. Levels of mutant p53 returned to baseline levels 24 h post each daily dose when plasma exposure fell below approximately 1000 ng/mL. At the higher dose of 100 mg/kg QD×1 a 26% and 42% reduction was observed in mutant p53 16 and 24 h post-dose, respectively. Repeat dosing of 100 mg/kg daily increased the reduction of mutant p53 to 86%, 80%, and 70% at 8 h post-dose on days 2, 4, and 6, respectively, which correlated with peak plasma concentrations of approximately 18,600 ng/mL at 8 h.

Administration of Compound 2 at 50 mg/kg QD×6 resulted in 1.8 and 2.3-fold increases in WT conformation p53 at 4 and 8 h, respectively, when plasma concentrations were approximately 14,000 ng/mL. Daily administration of Compound 2 at 50 mg/kg resulted in WT conformation p53 increases of 2.2-, 2.7-, and 2.0-fold at 8 h post-dose on days 2, 4, and 6, respectively, that decreased to baseline levels by 24 h post-dose. Tumors from mice administered 100 mg/kg QD×6 resulted in 2.6- and 3.5-fold increases in WT conformation p53 at 4 and 8 h on day 1, respectively, followed by increases of 3.1-, 3.7-, and 2.6-fold in WT conformation p53 at 8 h post-dose on days 2, 4, and 6, respectively, which correlated with peak plasma concentrations of Compound 2 of 18,600 ng/mL.

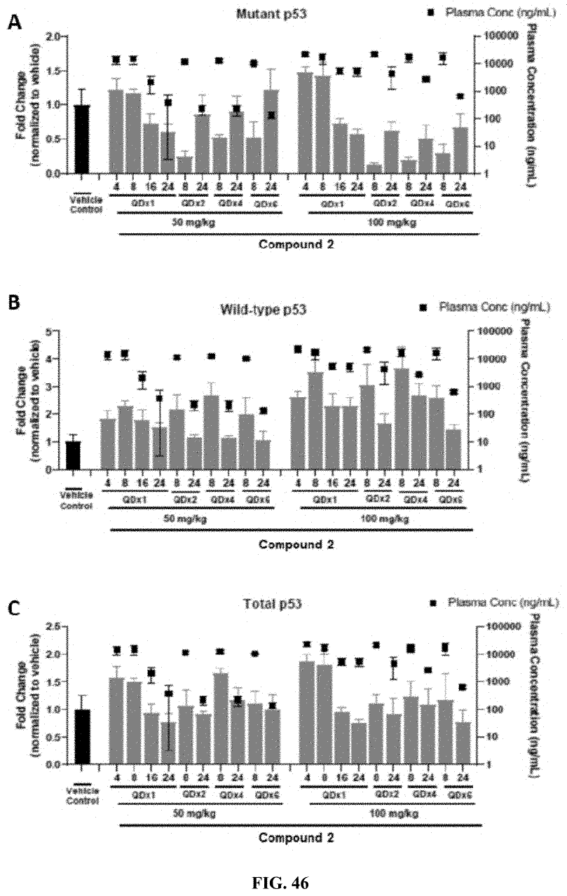

Levels of total p53 were slightly elevated (1.6 to 1.9-fold vehicle control) in both the 50 mg/kg and 100 mg/kg QD×1 dosing groups 4 and 8 h post the first dose, after which levels in both groups return to being comparable to the vehicle control levels across the course of the study. FIG. 46 PANEL A-PANEL C shows fold changes normalized to vehicle and plasma concentration (ng/mL) of mutant p53, WT conformation p53, and total p53 in mice treated with vehicle control, 50 mg/kg Compound 2 (QD×1, QD×2, QD×4, and QD×6), and 100 mg/kg Compound 2 (QD×1, QD×2, QD×4, and QD×6). TABLE 64 shows conversion of mutant p53 to wild-type conformation.

TABLE 64

| | | | | Fold Change over Vehicle Control or Percent Reduction | | | | | | | | |
|---|---|---|---|---|---|---|---|---|---|---|---|---|
| | | | | Relative to Vehicle Control (%) | | | | | | | | |
| Group - | | | Plasma | Mutant | WT | Total | | | | | | |
| Compound 2 | | Timepoint | Conc. | p53 | p53 | p53 | p21 | MDM2 | p21 | MDM2 | BIRC5 | MIC-1 |
| (mg/kg) | | (h) | (ng/mL) | Protein | Protein | Protein | Protein | Protein | mRNA | mRNA | mRNA | Plasma* |
| Group 2 - | QDx1 | 4 | 14425 | 1.22 | 1.83 | 1.57 | 2.07 | 5.53 | 2.88 | 2.62 | 1.68 | 0.94 |
| 50 QDx6 | | 8 | 14750 | 1.17 | 2.32 | 1.50 | 4.24 | 15.96 | 4.51 | 3.78 | 1.09 | 2.43 |
| | | 16 | 2101 | 26.0% | 1.81 | 6.0% | 3.58 | 8.52 | 2.43 | 2.40 | 31.1% | 2.58 |
| | | 24 | 368 | 39.0% | 1.52 | 22.0% | 1.58 | 2.43 | 1.40 | 1.47 | 32.7% | 1.01 |
| | QDx2 | 8 | 11553 | 75.0% | 2.18 | 1.07 | 4.29 | 14.04 | 5.06 | 3.06 | 44.4% | 3.19 |
| | | 24 | 218 | 14.0% | 1.18 | 9.0% | 1.14 | 1.83 | 1.28 | 1.55 | 1.01 | 1.02 |
| | QDx3 | 8 | 12725 | 47.0% | 2.69 | 1.65 | 5.04 | 14.31 | 5.28 | 3.40 | 1.58 | 3.51 |
| | | 24 | 212 | 9.0% | 1.14 | 1.16 | 1.21 | 1.94 | 1.07 | 1.35 | 9.3% | 1.62 |
| | QDx6 | 8 | 10270 | 47.0% | 1.99 | 1.11 | 2.39 | 7.56 | 6.07 | 2.86 | 13.6% | 3.68 |
| | | 24 | 137 | 1.22 | 1.04 | 0.0% | 19% | 21% | 1.10 | 1.07 | 2.6% | 1.18 |
| Group 3 - | QDx1 | 4 | 22875 | 1.47 | 2.61 | 1.88 | 3.78 | 14.64 | 5.82 | 4.29 | 1.34 | 1.14 |
| 100 QDx6 | | 8 | 16318 | 1.42 | 3.53 | 1.81 | 6.54 | 32.63 | 7.06 | 5.32 | 1.19 | 3.27 |
| | | 16 | 5578 | 26.0% | 2.30 | 5.0% | 3.64 | 10.32 | 2.66 | 2.72 | 42.0% | 2.43 |
| | | 24 | 5410 | 42.0% | 2.32 | 25.0% | 3.84 | 10.82 | 2.92 | 2.35 | 76.5% | 2.28 |
| | QDx2 | 8 | 21900 | 86.0% | 3.05 | 1.11 | 5.80 | 26.51 | 8.17 | 5.39 | 49.5% | 4.77 |
| | | 24 | 4470 | 38.0% | 1.70 | 8.0% | 2.18 | 4.19 | 3.25 | 2.18 | 34.3% | 2.10 |
| | QDx4 | 8 | 16878 | 80.0% | 3.68 | 1.23 | 5.49 | 21.79 | 11.89 | 5.03 | 11.4% | 5.26 |
| | | 24 | 2658 | 49.0% | 2.70 | 1.09 | 3.09 | 4.76 | 4.31 | 1.81 | 53.6% | 4.21 |
| | QDx6 | 8 | 17025 | 70.0% | 2.58 | 1.17 | 3.91 | 10.62 | 9.40 | 4.24 | 2.9% | 6.90 |
| | | 24 | 652 | 33.0% | 1.43 | 23.0% | 1.45 | 1.47 | 2.65 | 1.65 | 33.6% | 2.77 |

*= Absolute induction in pg/mL/mm³.

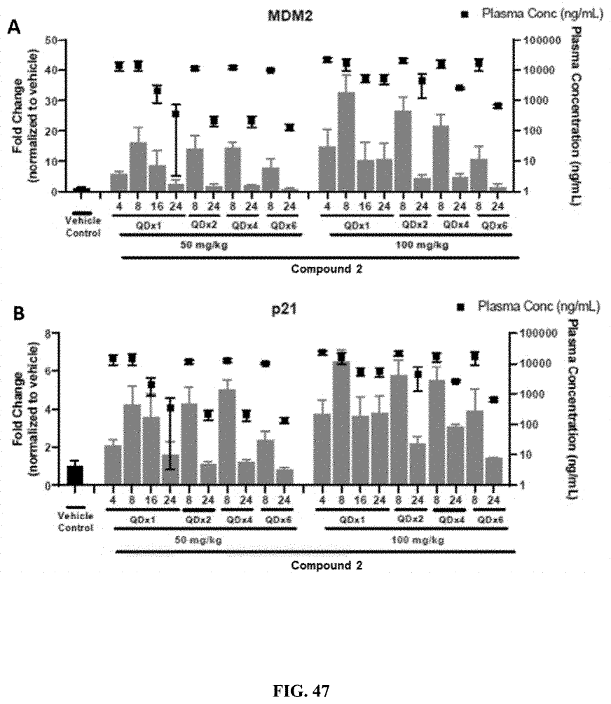

Measurement of p53 target proteins showed a 15.9-fold increase in MDM2 and a 4.2-fold increase in p21, both at 8 h post dose on day 1 in tumors from mice administered 50 mg/kg QD×6. The observation correlated with plasma concentrations of 14,000 ng/mL. As the Compound 2 plasma concentration peaked daily at 8 h post-dose (˜11,000 ng/mL), 14.0-, 14.3-, and 7.6-fold increases in MDM2 and 4.3-, 5.0-, and 2.4-fold increases in p21 were observed on days 2, 4, and 6, respectively. Tumors from mice administered with 100 mg/kg QD×6 resulted in increases of 32.6- and 6.5-fold in MDM2 and p21, respectively, at 8 h post dose on day 1. With daily dosing, as Compound 2 plasma concentrations increased to approximately 18,600 ng/mL, MDM2 levels increased 26.5-, 21.8-, and 10.6-fold, and p21 levels increased 5.8-, 5.5-, and 3.9-fold 8 h post-dose on days 2, 4, and 6, respectively. FIG. 47 PANEL A and PANEL B show fold changes normalized to vehicle and plasma concentration (ng/mL) of MDM2 and p21 in mice treated with vehicle control, 50 mg/kg Compound 2 (QD×1, QD×2, QD×4, and QD×6), and 100 mg/kg Compound 2 (QD×1, QD×2, QD×4, and QD×6). TABLE 64 shows wild-type p53 conversion resulted in downstream increases in p53 target proteins MDM2 and p21.

MIC-1 was also measured in the plasma of mice, normalized to tumor volume, as a circulating biomarker. Plasma from mice treated with the vehicle control averaged 0.44 pg/mL/mm³ levels of circulating MIC-1. Mice treated with Compound 2 at 50 mg/kg QD×6 had increased MIC-1 levels in the plasma of 2.58 pg/mL/mm³ 16 h following the first dose and 3.19, 3.51, and 3.68 pg/mL/mm³ 8 h post-dosing on days 2, 4, and 6, respectively. The values correlated with peak plasma concentrations of approximately 11,000 ng/mL of Compound 2. Administration of 100 mg/kg of Compound 2 QD×6 resulted in increases of 3.27, 4.77, 5.26, and 6.90 pg/mL/mm³ at 8 h on days 1, 2, 4, and 6, respectively, consistent with peak plasma exposures of Compound 2 (approximately 18,600 ng/mL) at the time points.

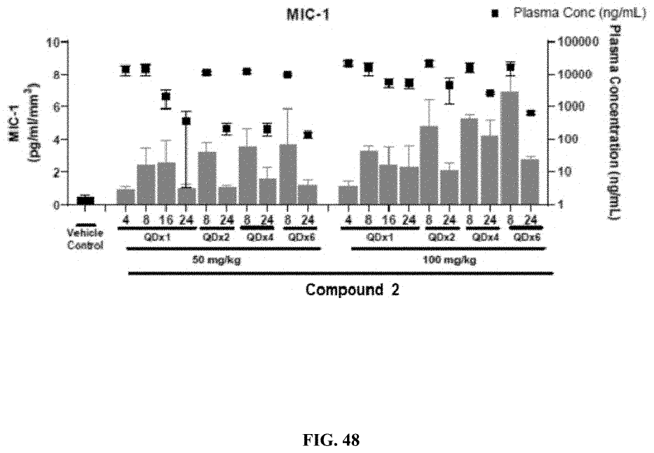

FIG. 48 shows MIC-1 (pg/mL/mm³) and plasma concentration (ng/mL) levels of mice treated with vehicle control, 50 mg/kg Compound 2 (QD×1, QD×2, QD×4, and QD×6), and 100 mg/kg Compound 2 (QD×1, QD×2, QD×4, and QD×6).

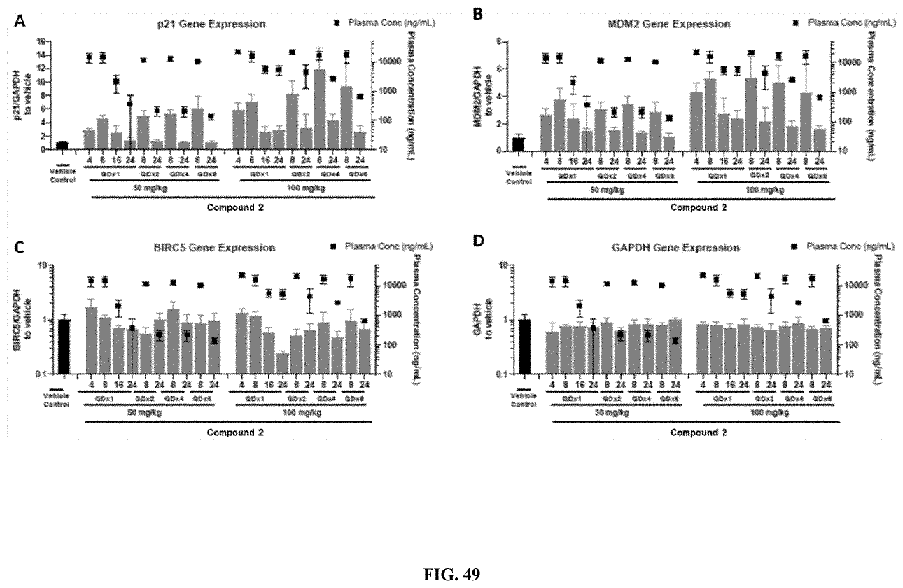

p53 dependent gene expression changes were analyzed initially for three p53 target gene mRNAs; p21, MDM2, and BIRC5 (Survivin). Following the first dose of Compound 2 at 50 mg/kg, p21 increased by 4.51-fold, and MDM2 increased by 3.78-fold, each at 8 h. The values returned to baseline by 24 h. A 32.7% reduction in BIRC5 was recorded at 24 h. Subsequent daily dosing resulted in increases of 5.06-, 5.28-, and 6.07-fold for p21, and increases of 3.06-, 3.40-, and 2.86-fold for MDM2 at 8 h on days 2, 4, and 6, respectively. Both markers returned to baseline levels by 24 h following dosing. A maximum 44.4% reduction in BIRC5 levels was observed at 8 h post-dose on day 2 with all other recorded values close to baseline. Administration of Compound 2 at 100 mg/kg resulted in increases of 7.06- and 5.32-fold in p21 and MDM2, respectively, at 8 h post-dose. Both levels remained elevated through 24 h at 2.92- and 2.35-fold, respectively. Compound 2 plasma concentration reached ˜16,000 ng/mL at 8 h and lowered to ˜5000 ng/mL by 24 h post-dose. Repeat dosing with Compound 2 at 100 mg/kg resulted in increases of 8.17-, 11.89-, and 9.40-fold for p21 and 5.39-, 5.03-, and 4.24-fold for MDM2 at 8 h on day 2, day 4, and day 6, respectively. A 76.5% reduction in BIRC5 mRNA expression was observed at 24 h following initial dose, along with 34.3, 53.6, and 33.6% reductions recorded at 24 h post-dose on days 2, 4, and 6, respectively. FIG. 49 PANEL A-PANEL D show p21, MDM2, BIRC5, and GAPDH gene expression changes relative to vehicle and plasma concentrations (ng/mL) in mice treated with vehicle control, 50 mg/kg Compound 2 (QD×1, QD×2, QD×4, and QD×6), and 100 mg/kg Compound 2 (QD×1, QD×2, QD×4, and QD×6). TABLE 64 shows changes in p21, MDM2, and BIRC5 (survivin) gene expression relative to GAPDH following daily dosing of Compound 2.

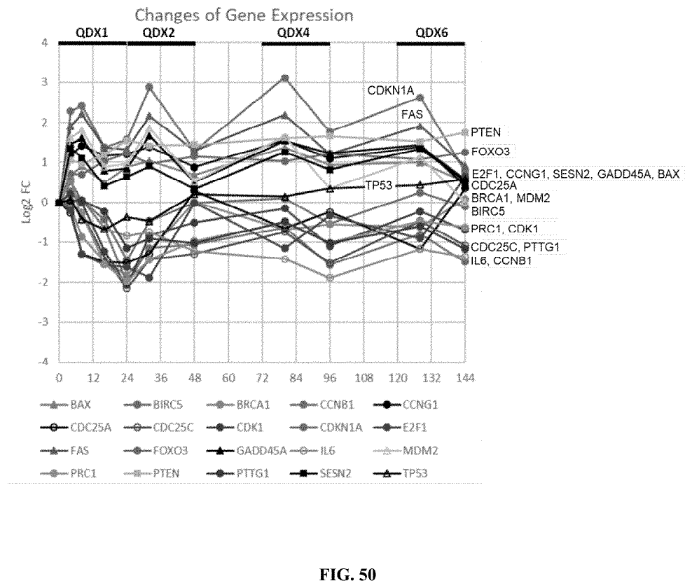

A larger panel of p53 target genes was assessed to understand gene expression changes across time in the tumors from mice dosed with 100 mg/kg QD×6 only. Following administration of Compound 2 at 100 mg/kg QD×6 the largest fold change following each dose administration occurred around 8 h post-dose with changes returning toward baseline by 24 h post-dose. A representative 20 out of 84 genes are shown in FIG. 50 and TABLE 65 to demonstrate the p53-related pathway upregulated or downregulated gene expression changes. Levels of p53 mRNAs did not change across the course of the study. Gene expression increases appeared immediately following administration of 100 mg/kg with peak changes occurring approximately 8 h post-the first dose and 8 h post subsequent doses. Two mRNAs exhibiting the largest fold increases 8 h post dose were p21 (CDKN1A; 5.8- and 9.3-fold, respectively) and MDM2 (4.0- and 4.3-fold, respectively). Decreases in selected genes mRNA expression were observed at 24 h post the first dose of 100 mg/kg Compound 2 where CCNB1, CDC25C, CDK1, and BIRC5 were reduced by 77%, 76%, 76%, and 72%, respectively. Negatively regulated gene expression did not return to baseline by 24 h post-dose and remained suppressed.

TABLE 65

| | | Compound 2 100 mg/kg QDx6 Fold Change Compared to Vehicle Control | | | | | | | | | |
|---|---|---|---|---|---|---|---|---|---|---|---|
| | Vehicle | QDx1 | QDx1 | QDx1 | QDx1 | QDx2 | QDx2 | QDx4 | QDx4 | QDx6 | QDx6 |
| Gene Name | Control | 4 h | 8 h | 16 h | 24 h | 8 h | 24 h | 8 h | 24 h | 8 h | 24 h |
| ARAF1 | 1.00 ± | 1.39 ± | 1.14 ± | 1.07 ± | 1.04 ± | 1.12 ± | 1.01 ± | 1.22 ± | 1.01 ± | 1.35 ± | 1.27 ± |
| | 0.22 | 0.23 | 0.21 | 0.24 | 0.21 | 0.25 | 0.21 | 0.21 | 0.21 | 0.21 | 0.22 |
| ATM | 1.00 ± | 1.08 ± | 0.87 ± | 1.22 ± | 1.23 ± | 1.23 ± | 1.52 ± | 1.17 ± | 0.99 ± | 1.35 ± | 1.32 ± |
| | 0.24 | 0.23 | 0.22 | 0.26 | 0.24 | 0.27 | 0.27 | 0.23 | 0.23 | 0.22 | 0.24 |
| ATR | 1.00 ± | 1.20 ± | 1.03 ± | 1.39 ± | 1.29 ± | 1.33 ± | 1.66 ± | 1.40 ± | 1.21 ± | 1.22 ± | 1.39 ± |
| | 0.29 | 0.27 | 0.27 | 0.30 | 0.31 | 0.30 | 0.32 | 0.27 | 0.27 | 0.27 | 0.27 |
| BAL1 | 1.00 ± | 1.15 ± | 1.23 ± | 2.25 ± | 2.50 ± | 1.48 ± | 1.95 ± | 1.80 ± | 1.61 ± | 1.59 ± | 1.24 ± |
| | 0.52 | 0.49 | 0.48 | 0.50 | 0.63 | 0.56 | 0.52 | 0.51 | 0.49 | 0.52 | 0.49 |
| BAX | 1.00 ± | 1.75 ± | 2.05 ± | 1.55 ± | 2.04 ± | 2.23 ± | 1.81 ± | 2.82 ± | 2.07 ± | 2.16 ± | 1.55 ± |
| | 0.30 | 0.28 | 0.30 | 0.30 | 0.32 | 0.28 | 0.34 | 0.29 | 0.28 | 0.28 | 0.28 |
| BBC3 | 1.00 ± | 2.53 ± | 2.10 ± | 1.08 ± | 1.42 ± | 2.06 ± | 1.32 ± | 2.63 ± | 2.01 ± | 2.70 ± | 1.54 ± |
| | 0.23 | 0.30 | 0.31 | 0.26 | 0.26 | 0.28 | 0.28 | 0.22 | 0.26 | 0.29 | 0.24 |
| BCL2 | 1.00 ± | 0.95 ± | 0.74 ± | 1.15 ± | 1.08 ± | 0.87 ± | 1.23 ± | 1.01 ± | 0.95 ± | 1.45 ± | 1.17 ± |
| | 0.20 | 0.20 | 0.19 | 0.25 | 0.20 | 0.18 | 0.21 | 0.22 | 0.19 | 0.47 | 0.19 |
| BCL2A1 | 1.00 ± | 1.27 ± | 2.11 ± | 1.78 ± | 1.23 ± | 2.10 ± | 0.57 ± | 0.81 ± | 0.39 ± | 0.51 ± | 0.72 ± |
| | 0.49 | 0.56 | 0.49 | 0.55 | 0.51 | 0.58 | 0.47 | 0.47 | 0.46 | 0.45 | 0.46 |
| BID | 1.00 ± | 1.03 ± | 0.94 ± | 0.86 ± | 1.05 ± | 0.99 ± | 1.32 ± | 1.28 ± | 1.34 ± | 1.25 ± | 1.70 ± |
| | 0.15 | 0.14 | 0.16 | 0.14 | 0.14 | 0.16 | 0.14 | 0.14 | 0.14 | 0.15 | 0.15 |
| BIRC5 | 1.00 ± | 1.08 ± | 0.77 ± | 0.55 ± | 0.28 ± | 0.56 ± | 0.74 ± | 0.82 ± | 0.53 ± | 0.96 ± | 0.72 ± |
| | 0.31 | 0.30 | 0.29 | 0.29 | 0.29 | 0.29 | 0.29 | 0.31 | 0.29 | 0.34 | 0.31 |
| BRCA1 | 1.00 ± | 1.02 ± | 0.61 ± | 0.40 ± | 0.32 ± | 0.49 ± | 1.15 ± | 0.70 ± | 0.79 ± | 0.78 ± | 1.22 ± |
| | 0.38 | 0.35 | 0.34 | 0.34 | 0.34 | 0.35 | 0.37 | 0.35 | 0.36 | 0.37 | 0.38 |
| BRCA2 | 1.00 ± | 1.04 ± | 0.75 ± | 0.54 ± | 0.37 ± | 0.62 ± | 1.07 ± | 0.81 ± | 0.76 ± | 0.87 ± | 1.42 ± |
| | 0.32 | 0.29 | 0.29 | 0.29 | 0.30 | 0.30 | 0.30 | 0.31 | 0.30 | 0.33 | 0.35 |
| BTG2 | 1.00 ± | 8.70 ± | 6.73 ± | 2.57 ± | 3.19 ± | 5.56 ± | 2.29 ± | 7.88 ± | 4.04 ± | 8.37 ± | 2.02 ± |
| | 0.44 | 0.78 | 1.22 | 0.49 | 0.61 | 0.91 | 0.54 | 0.68 | 0.59 | 1.31 | 0.45 |
| CASP2 | 1.00 ± | 0.86 ± | 0.58 ± | 0.72 ± | 0.85 ± | 0.70 ± | 1.19 ± | 0.97 ± | 1.19 ± | 1.13 ± | 1.70 ± |
| | 0.34 | 0.31 | 0.31 | 0.31 | 0.31 | 0.31 | 0.32 | 0.31 | 0.32 | 0.31 | 0.32 |
| CASP9 | 1.00 ± | 1.13 ± | 0.97 ± | 1.26 ± | 1.19 ± | 1.07 ± | 1.42 ± | 1.08 ± | 1.09 ± | 1.19 ± | 1.24 ± |
| | 0.20 | 0.20 | 0.19 | 0.21 | 0.22 | 0.19 | 0.25 | 0.20 | 0.19 | 0.19 | 0.19 |
| CCNB1 | 1.00 ± | 1.13 ± | 0.98 ± | 0.59 ± | 0.23 ± | 0.45 ± | 0.45 ± | 0.62 ± | 0.33 ± | 0.57 ± | 0.33 ± |
| | 0.24 | 0.24 | 0.23 | 0.22 | 0.22 | 0.23 | 0.22 | 0.23 | 0.22 | 0.24 | 0.22 |
| CCNE1 | 1.00 ± | 0.87 ± | 0.58 ± | 1.01 ± | 0.98 ± | 0.85 ± | 1.67 ± | 0.97 ± | 1.37 ± | 0.69 ± | 1.58 ± |
| | 0.26 | 0.25 | 0.24 | 0.25 | 0.26 | 0.24 | 0.30 | 0.25 | 0.28 | 0.26 | 0.32 |
| CCNG1 | 1.00 ± | 2.16 ± | 2.56 ± | 2.17 ± | 2.20 ± | 2.48 ± | 1.78 ± | 2.79 ± | 2.04 ± | 2.52 ± | 1.41 ± |
| | 0.17 | 0.18 | 0.22 | 0.28 | 0.20 | 0.17 | 0.23 | 0.16 | 0.19 | 0.24 | 0.17 |
| CCNH | 1.00 ± | 0.96 ± | 1.00 ± | 1.26 ± | 1.23 ± | 1.15 ± | 1.24 ± | 1.06 ± | 0.99 ± | 0.95 ± | 0.98 ± |
| | 0.32 | 0.29 | 0.30 | 0.31 | 0.31 | 0.30 | 0.32 | 0.29 | 0.29 | 0.29 | 0.29 |
| CDC25A | 1.00 ± | 0.94 ± | 0.46 ± | 0.40 ± | 0.42 ± | 0.46 ± | 1.39 ± | 0.74 ± | 1.01 ± | 0.60 ± | 1.53 ± |
| | 0.33 | 0.31 | 0.30 | 0.30 | 0.31 | 0.30 | 0.33 | 0.31 | 0.35 | 0.33 | 0.40 |
| CDC25C | 1.00 ± | 1.03 ± | 0.87 ± | 0.41 ± | 0.24 ± | 0.42 ± | 0.43 ± | 0.65 ± | 0.37 ± | 0.81 ± | 0.50 ± |
| | 0.31 | 0.30 | 0.29 | 0.28 | 0.28 | 0.30 | 0.28 | 0.29 | 0.29 | 0.31 | 0.29 |
| CDK1 | 1.00 ± | 1.18 ± | 1.01 ± | 0.43 ± | 0.24 ± | 0.61 ± | 0.72 ± | 0.94 ± | 0.50 ± | 0.95 ± | 0.67 ± |
| | 0.13 | 0.14 | 0.13 | 0.12 | 0.12 | 0.16 | 0.14 | 0.18 | 0.14 | 0.22 | 0.18 |
| CDK4 | 1.00 ± | 0.96 ± | 0.77 ± | 0.63 ± | 0.75 ± | 0.76 ± | 1.02 ± | 0.93 ± | 1.01 ± | 0.88 ± | 1.01 ± |
| | 0.26 | 0.24 | 0.24 | 0.23 | 0.24 | 0.24 | 0.24 | 0.24 | 0.25 | 0.24 | 0.24 |
| CDKN1A | 1.00 ± | 5.21 ± | 5.75 ± | 2.95 ± | 3.32 ± | 7.88 ± | 3.03 ± | 9.28 ± | 3.67 ± | 6.72 ± | 1.94 ± |
| | 0.23 | 0.36 | 0.40 | 0.53 | 0.48 | 0.36 | 0.87 | 0.41 | 0.33 | 0.65 | 0.39 |
| CDKN2A | 1.00 ± | 0.91 ± | 0.91 ± | 1.09 ± | 1.11 ± | 1.11 ± | 1.29 ± | 1.14 ± | 1.10 ± | 1.27 ± | 1.38 ± |
| | 0.15 | 0.14 | 0.15 | 0.16 | 0.15 | 0.15 | 0.15 | 0.15 | 0.15 | 0.15 | 0.15 |
| CHEK1 | 1.00 ± | 1.05 ± | 0.75 ± | 0.54 ± | 0.49 ± | 0.60 ± | 1.01 ± | 0.69 ± | 0.69 ± | 0.65 ± | 1.01 ± |
| | 0.19 | 0.18 | 0.18 | 0.17 | 0.18 | 0.18 | 0.19 | 0.19 | 0.20 | 0.21 | 0.22 |
| CHEK2 | 1.00 ± | 1.06 ± | 0.93 ± | 0.83 ± | 0.68 ± | 0.77 ± | 0.97 ± | 0.81 ± | 0.55 ± | 0.83 ± | 0.77 ± |
| | 0.26 | 0.24 | 0.24 | 0.24 | 0.25 | 0.25 | 0.24 | 0.25 | 0.24 | 0.26 | 0.25 |
| CRADD | 1.00 ± | 1.11 ± | 1.07 ± | 1.26 ± | 1.23 ± | 1.11 ± | 1.16 ± | 1.15 ± | 1.14 ± | 1.18 ± | 1.09 ± |
| | 0.11 | 0.10 | 0.11 | 0.12 | 0.11 | 0.11 | 0.10 | 0.11 | 0.12 | 0.11 | 0.11 |
| DNMT1 | 1.00 ± | 1.03 ± | 0.74 ± | 0.61 ± | 0.57 ± | 0.70 ± | 1.06 ± | 0.82 ± | 0.79 ± | 0.72 ± | 0.86 ± |
| | 0.33 | 0.31 | 0.30 | 0.30 | 0.30 | 0.31 | 0.32 | 0.31 | 0.31 | 0.32 | 0.31 |
| E2F1 | 1.00 ± | 0.95 ± | 0.44 ± | 0.37 ± | 0.36 ± | 0.29 ± | 1.10 ± | 0.52 ± | 0.89 ± | 0.69 ± | 1.78 ± |
| | 0.41 | 0.38 | 0.37 | 0.37 | 0.37 | 0.37 | 0.40 | 0.38 | 0.39 | 0.41 | 0.48 |
| E2F3 | 1.00 ± | 1.17 ± | 1.03 ± | 0.96 ± | 0.86 ± | 1.01 ± | 1.29 ± | 1.26 ± | 1.06 ± | 1.02 ± | 1.24 ± |
| | 0.32 | 0.29 | 0.30 | 0.30 | 0.30 | 0.31 | 0.32 | 0.30 | 0.30 | 0.30 | 0.31 |
| EGFR | 1.00 ± | 0.86 ± | 0.82 ± | 0.98 ± | 1.13 ± | 1.01 ± | 0.92 ± | 0.89 ± | 0.91 ± | 0.86 ± | 0.81 ± |
| | 0.27 | 0.25 | 0.25 | 0.27 | 0.26 | 0.26 | 0.25 | 0.25 | 0.27 | 0.25 | 0.26 |
| EGR1 | 1.00 ± | 0.91 ± | 0.59 ± | 0.62 ± | 0.49 ± | 0.51 ± | 0.39 ± | 0.24 ± | 0.33 ± | 0.37 ± | 0.35 ± |
| | 0.70 | 0.65 | 0.64 | 0.64 | 0.64 | 0.64 | 0.63 | 0.63 | 0.64 | 0.64 | 0.64 |
| EI24 | 1.00 ± | 1.24 ± | 1.34 ± | 1.17 ± | 1.42 ± | 1.43 ± | 1.30 ± | 1.72 ± | 1.56 ± | 1.40 ± | 1.26 ± |
| | 0.17 | 0.16 | 0.18 | 0.16 | 0.19 | 0.21 | 0.17 | 0.17 | 0.16 | 0.15 | 0.15 |
| ESR1 | 1.00 ± | 0.70 ± | 0.70 ± | 1.18 ± | 1.51 ± | 1.03 ± | 1.18 ± | 1.30 ± | 1.55 ± | 1.77 ± | 1.63 ± |
| | 0.34 | 0.31 | 0.33 | 0.33 | 0.33 | 0.34 | 0.31 | 0.33 | 0.32 | 0.35 | 0.33 |
| FADD | 1.00 ± | 1.03 ± | 0.89 ± | 0.85 ± | 1.19 ± | 0.99 ± | 1.28 ± | 1.20 ± | 1.41 ± | 1.31 ± | 1.25 ± |
| | 0.28 | 0.27 | 0.27 | 0.26 | 0.27 | 0.28 | 0.26 | 0.26 | 0.26 | 0.26 | 0.27 |
| FAS | 1.00 ± | 2.67 ± | 3.29 ± | 1.90 ± | 1.79 ± | 3.19 ± | 1.78 ± | 3.23 ± | 1.64 ± | 2.71 ± | 1.33 ± |
| | 0.34 | 0.33 | 0.35 | 0.40 | 0.34 | 0.37 | 0.37 | 0.36 | 0.31 | 0.42 | 0.31 |
| FASLG | 1.00 ± | 0.66 ± | 0.74 ± | 1.26 ± | 1.44 ± | 0.98 ± | 0.97 ± | 1.10 ± | 0.97 ± | 1.20 ± | 1.38 ± |
| | 0.34 | 0.31 | 0.32 | 0.36 | 0.35 | 0.36 | 0.32 | 0.35 | 0.32 | 0.32 | 0.33 |
| FOXO3 | 1.00 ± | 0.90 ± | 0.89 ± | 1.12 ± | 1.30 ± | 1.09 ± | 1.22 ± | 1.13 ± | 1.27 ± | 1.17 ± | 1.30 ± |
| | 0.32 | 0.29 | 0.29 | 0.29 | 0.30 | 0.31 | 0.29 | 0.30 | 0.29 | 0.30 | 0.29 |
| GADD45A | 1.00 ± | 2.20 ± | 2.45 ± | 1.38 ± | 1.47 ± | 2.52 ± | 1.15 ± | 2.31 ± | 1.87 ± | 2.19 ± | 1.06 ± |
| | 0.29 | 0.28 | 0.32 | 0.28 | 0.30 | 0.28 | 0.29 | 0.28 | 0.29 | 0.34 | 0.27 |
| GML | 1.00 ± | 0.99 ± | 1.01 ± | 0.99 ± | 0.99 ± | 1.14 ± | 1.01 ± | 0.99 ± | 0.99 ± | 1.00 ± | 1.07 ± |
| | 0.04 | 0.03 | 0.04 | 0.03 | 0.03 | 0.13 | 0.04 | 0.03 | 0.03 | 0.04 | 0.08 |
| HDAC1 | 1.00 ± | 0.97 ± | 0.87 ± | 0.88 ± | 0.99 ± | 0.87 ± | 1.05 ± | 0.97 ± | 1.08 ± | 0.99 ± | 1.07 ± |
| | 0.28 | 0.26 | 0.26 | 0.26 | 0.26 | 0.26 | 0.26 | 0.26 | 0.26 | 0.26 | 0.26 |
| HK2 | 1.00 ± | 0.72 ± | 0.78 ± | 0.75 ± | 0.79 ± | 0.73 ± | 0.73 ± | 0.59 ± | 0.66 ± | 0.64 ± | 0.77 ± |
| | 0.27 | 0.25 | 0.27 | 0.26 | 0.28 | 0.26 | 0.26 | 0.25 | 0.25 | 0.25 | 0.25 |
| IGF1R | 1.00 ± | 1.05 ± | 0.87 ± | 0.84 ± | 1.01 ± | 0.96 ± | 1.07 ± | 1.09 ± | 1.14 ± | 1.24 ± | 1.29 ± |
| | 0.28 | 0.26 | 0.26 | 0.27 | 0.26 | 0.27 | 0.26 | 0.26 | 0.26 | 0.27 | 0.26 |
| IL6 | 1.00 ± | 1.79 ± | 0.92 ± | 0.78 ± | 0.68 ± | 0.73 ± | 0.52 ± | 0.44 ± | 0.32 ± | 0.56 ± | 0.47 ± |
| | 0.41 | 0.44 | 0.42 | 0.40 | 0.39 | 0.39 | 0.38 | 0.38 | 0.38 | 0.39 | 0.38 |
| JUN | 1.00 ± | 0.68 ± | 0.67 ± | 0.44 ± | 0.47 ± | 0.41 ± | 0.40 ± | 0.33 ± | 0.40 ± | 0.40 ± | 0.46 ± |
| | 0.59 | 0.53 | 0.55 | 0.54 | 0.54 | 0.53 | 0.53 | 0.53 | 0.53 | 0.53 | 0.53 |
| KAT2B | 1.00 ± | 0.97 ± | 0.88 ± | 1.14 ± | 1.26 ± | 1.07 ± | 1.21 ± | 1.11 ± | 1.34 ± | 1.49 ± | 1.70 ± |
| | 0.31 | 0.28 | 0.28 | 0.29 | 0.32 | 0.29 | 0.28 | 0.29 | 0.29 | 0.31 | 0.32 |
| KRAS | 1.00 ± | 1.02 ± | 0.87 ± | 1.05 ± | 1.03 ± | 1.04 ± | 1.25 ± | 1.11 ± | 1.14 ± | 1.07 ± | 1.30 ± |
| | 0.24 | 0.23 | 0.23 | 0.23 | 0.23 | 0.23 | 0.23 | 0.22 | 0.23 | 0.22 | 0.23 |
| MCL1 | 1.00 ± | 1.12 ± | 1.19 ± | 1.19 ± | 1.16 ± | 1.31 ± | 1.14 ± | 1.04 ± | 0.90 ± | 0.88 ± | 0.86 ± |
| | 0.26 | 0.24 | 0.25 | 0.27 | 0.27 | 0.27 | 0.26 | 0.24 | 0.24 | 0.24 | 0.24 |
| MDM2 | 1.00 ± | 3.52 ± | 3.99 ± | 2.23 ± | 2.27 ± | 4.32 ± | 1.73 ± | 3.59 ± | 1.46 ± | 2.59 ± | 1.16 ± |
| | 0.41 | 0.43 | 0.40 | 0.50 | 0.42 | 0.75 | 0.48 | 0.40 | 0.38 | 0.51 | 0.38 |
| MDM4 | 1.00 ± | 1.11 ± | 1.22 ± | 1.26 ± | 1.31 ± | 1.34 ± | 1.13 ± | 1.38 ± | 1.02 ± | 1.55 ± | 1.25 ± |
| | 0.28 | 0.26 | 0.26 | 0.29 | 0.26 | 0.29 | 0.26 | 0.26 | 0.27 | 0.29 | 0.26 |
| MLH1 | 1.00 ± | 1.12 ± | 0.97 ± | 0.92 ± | 0.94 ± | 0.98 ± | 1.18 ± | 1.29 ± | 1.12 ± | 1.21 ± | 1.30 ± |
| | 0.22 | 0.22 | 0.21 | 0.21 | 0.21 | 0.21 | 0.22 | 0.22 | 0.21 | 0.21 | 0.21 |
| MSH2 | 1.00 ± | 0.99 ± | 0.71 ± | 0.94 ± | 0.85 ± | 0.83 ± | 1.36 ± | 0.86 ± | 0.93 ± | 0.89 ± | 1.26 ± |
| | 0.35 | 0.32 | 0.32 | 0.32 | 0.33 | 0.32 | 0.34 | 0.32 | 0.32 | 0.33 | 0.32 |
| MYC | 1.00 ± | 0.87 ± | 0.84 ± | 1.07 ± | 1.17 ± | 1.09 ± | 1.19 ± | 1.11 ± | 1.03 ± | 0.80 ± | 0.78 ± |
| | 0.39 | 0.36 | 0.36 | 0.36 | 0.40 | 0.36 | 0.38 | 0.36 | 0.36 | 0.36 | 0.36 |
| MYOD1 | 1.00 ± | 1.02 ± | 1.02 ± | 1.02 ± | 1.02 ± | 1.02 ± | 1.02 ± | 1.02 ± | 1.02 ± | 1.02 ± | 0.99 ± |
| | 0.08 | 0.08 | 0.08 | 0.08 | 0.08 | 0.08 | 0.08 | 0.08 | 0.08 | 0.08 | 0.08 |
| NF1 | 1.00 ± | 1.15 ± | 1.01 ± | 1.20 ± | 1.17 ± | 1.17 ± | 1.27 ± | 1.08 ± | 0.92 ± | 1.02 ± | 1.10 ± |
| | 0.25 | 0.23 | 0.24 | 0.27 | 0.26 | 0.27 | 0.27 | 0.23 | 0.23 | 0.23 | 0.23 |
| NFKB1 | 1.00 ± | 1.04 ± | 0.89 ± | 0.96 ± | 1.02 ± | 1.00 ± | 1.15 ± | 1.05 ± | 1.09 ± | 1.07 ± | 1.18 ± |
| | 0.23 | 0.21 | 0.21 | 0.22 | 0.21 | 0.22 | 0.22 | 0.21 | 0.21 | 0.21 | 0.21 |
| PCNA | 1.00 ± | 1.17 ± | 1.02 ± | 0.62 ± | 0.60 ± | 0.87 ± | 1.28 ± | 1.23 ± | 1.07 ± | 1.22 ± | 1.51 ± |
| | 0.19 | 0.18 | 0.18 | 0.17 | 0.17 | 0.18 | 0.19 | 0.22 | 0.21 | 0.26 | 0.27 |
| PIDD | 1.00 ± | 1.79 ± | 1.69 ± | 1.34 ± | 1.56 ± | 1.35 ± | 1.61 ± | 2.14 ± | 1.56 ± | 1.65 ± | 1.17 ± |
| | 0.29 | 0.31 | 0.29 | 0.32 | 0.26 | 0.27 | 0.38 | 0.29 | 0.26 | 0.30 | 0.28 |
| PPM1D | 1.00 ± | 1.94 ± | 1.79 ± | 1.37 ± | 1.40 ± | 1.73 ± | 1.44 ± | 2.02 ± | 1.44 ± | 1.96 ± | 1.62 ± |
| | 0.25 | 0.25 | 0.31 | 0.28 | 0.24 | 0.23 | 0.27 | 0.26 | 0.23 | 0.25 | 0.23 |
| PRC1 | 1.00 ± | 1.02 ± | 0.84 ± | 0.34 ± | 0.26 ± | 0.38 ± | 0.52 ± | 0.73 ± | 0.52 ± | 0.78 ± | 0.68 ± |
| | 0.23 | 0.24 | 0.23 | 0.21 | 0.21 | 0.22 | 0.22 | 0.23 | 0.23 | 0.25 | 0.24 |
| PRKCA | 1.00 ± | 1.02 ± | 0.89 ± | 0.97 ± | 1.16 ± | 1.13 ± | 1.10 ± | 0.98 ± | 1.25 ± | 1.09 ± | 1.27 ± |
| | 0.25 | 0.23 | 0.24 | 0.25 | 0.26 | 0.23 | 0.24 | 0.23 | 0.23 | 0.23 | 0.27 |
| PTEN | 1.00 ± | 1.12 ± | 1.05 ± | 1.23 ± | 1.56 ± | 1.43 ± | 1.46 ± | 1.63 ± | 1.70 ± | 1.54 ± | 1.80 ± |
| | 0.32 | 0.29 | 0.30 | 0.30 | 0.29 | 0.29 | 0.30 | 0.29 | 0.32 | 0.29 | 0.29 |
| PTTG1 | 1.00 ± | 1.02 ± | 1.03 ± | 0.85 ± | 0.45 ± | 0.54 ± | 0.49 ± | 0.72 ± | 0.49 ± | 0.68 ± | 0.45 ± |
| | 0.18 | 0.17 | 0.17 | 0.17 | 0.16 | 0.17 | 0.16 | 0.17 | 0.16 | 0.18 | 0.17 |
| RB1 | 1.00 ± | 1.13 ± | 0.96 ± | 0.91 ± | 0.85 ± | 1.02 ± | 1.29 ± | 1.10 ± | 0.94 ± | 0.96 ± | 1.11 ± |
| | 0.30 | 0.28 | 0.28 | 0.28 | 0.27 | 0.28 | 0.30 | 0.28 | 0.28 | 0.27 | 0.27 |
| RELA | 1.00 ± | 0.93 ± | 0.88 ± | 0.99 ± | 1.08 ± | 0.94 ± | 1.02 ± | 0.88 ± | 0.97 ± | 0.90 ± | 1.05 ± |
| | 0.21 | 0.20 | 0.20 | 0.23 | 0.21 | 0.20 | 0.20 | 0.20 | 0.20 | 0.20 | 0.20 |
| RPRM | 1.00 ± | 1.39 ± | 1.30 ± | 1.71 ± | 1.41 ± | 1.57 ± | 1.93 ± | 1.52 ± | 1.19 ± | 1.45 ± | 1.45 ± |
| | 0.48 | 0.46 | 0.44 | 0.52 | 0.49 | 0.54 | 0.60 | 0.46 | 0.46 | 0.45 | 0.48 |
| SESN2 | 1.00 ± | 2.58 ± | 2.18 ± | 1.35 ± | 1.59 ± | 1.91 ± | 1.28 ± | 2.40 ± | 1.78 ± | 2.57 ± | 1.45 ± |
| | 0.25 | 0.25 | 0.26 | 0.26 | 0.26 | 0.28 | 0.27 | 0.24 | 0.25 | 0.30 | 0.24 |
| SIAH1 | 1.00 ± | 0.94 ± | 0.95 ± | 1.09 ± | 1.02 ± | 1.03 ± | 1.05 ± | 0.87 ± | 0.86 ± | 0.85 ± | 0.96 ± |
| | 0.23 | 0.21 | 0.21 | 0.22 | 0.21 | 0.23 | 0.21 | 0.21 | 0.21 | 0.21 | 0.21 |
| SIRT1 | 1.00 ± | 0.98 ± | 0.75 ± | 1.05 ± | 1.23 ± | 0.90 ± | 1.09 ± | 0.97 ± | 0.97 ± | 0.98 ± | 1.08 ± |
| | 0.16 | 0.15 | 0.15 | 0.16 | 0.17 | 0.16 | 0.16 | 0.15 | 0.15 | 0.16 | 0.15 |
| STAT1 | 1.00 ± | 1.10 ± | 0.85 ± | 0.76 ± | 0.69 ± | 0.74 ± | 0.99 ± | 1.22 ± | 1.33 ± | 1.43 ± | 1.87 ± |
| | 0.25 | 0.25 | 0.24 | 0.24 | 0.23 | 0.23 | 0.24 | 0.25 | 0.24 | 0.25 | 0.25 |
| TADA3 | 1.00 ± | 0.96 ± | 0.83 ± | 0.95 ± | 1.08 ± | 0.96 ± | 1.11 ± | 0.98 ± | 1.11 ± | 1.07 ± | 1.18 ± |
| | 0.20 | 0.19 | 0.19 | 0.18 | 0.19 | 0.19 | 0.18 | 0.18 | 0.18 | 0.19 | 0.19 |
| TNF | 1.00 ± | 0.99 ± | 0.57 ± | 0.87 ± | 1.04 ± | 0.84 ± | 0.86 ± | 0.51 ± | 0.57 ± | 0.70 ± | 0.68 ± |
| | 0.30 | 0.36 | 0.30 | 0.30 | 0.31 | 0.29 | 0.30 | 0.30 | 0.28 | 0.28 | 0.29 |
| TNFRSF10B | 1.00 ± | 1.79 ± | 1.98 ± | 1.32 ± | 1.35 ± | 1.81 ± | 1.27 ± | 1.84 ± | 1.19 ± | 1.47 ± | 0.91 ± |
| | 0.16 | 0.15 | 0.14 | 0.20 | 0.16 | 0.19 | 0.18 | 0.15 | 0.15 | 0.18 | 0.15 |
| TNFRSF10D | 1.00 ± | 1.34 ± | 1.50 ± | 1.21 ± | 1.37 ± | 1.55 ± | 1.20 ± | 1.33 ± | 0.94 ± | 1.08 ± | 0.81 ± |
| | 0.14 | 0.14 | 0.13 | 0.17 | 0.16 | 0.14 | 0.14 | 0.13 | 0.13 | 0.14 | 0.15 |
| TP53 | 1.00 ± | 0.95 ± | 0.68 ± | 0.57 ± | 0.70 ± | 0.65 ± | 1.04 ± | 1.00 ± | 1.15 ± | 1.22 ± | 1.35 ± |
| | 0.22 | 0.21 | 0.20 | 0.20 | 0.20 | 0.20 | 0.20 | 0.21 | 0.22 | 0.20 | 0.20 |
| TP53AIP1 | 1.00 ± | 1.65 ± | 1.70 ± | 2.38 ± | 2.07 ± | 2.22 ± | 2.29 ± | 2.62 ± | 2.06 ± | 2.12 ± | 2.37 ± |
| | 0.44 | 0.40 | 0.45 | 0.48 | 0.43 | 0.46 | 0.53 | 0.41 | 0.43 | 0.44 | 0.48 |
| TP53BP2 | 1.00 ± | 0.93 ± | 0.85 ± | 1.05 ± | 1.05 ± | 0.94 ± | 1.05 ± | 0.85 ± | 0.85 ± | 0.84 ± | 0.92 ± |
| | 0.15 | 0.14 | 0.14 | 0.16 | 0.14 | 0.15 | 0.16 | 0.14 | 0.14 | 0.14 | 0.14 |
| TP63 | 1.00 ± | 0.83 ± | 0.60 ± | 0.78 ± | 0.89 ± | 0.75 ± | 0.93 ± | 0.76 ± | 1.10 ± | 0.88 ± | 0.83 ± |
| | 0.14 | 0.13 | 0.13 | 0.14 | 0.13 | 0.14 | 0.13 | 0.13 | 0.17 | 0.13 | 0.13 |
| TP73 | 1.00 ± | 0.86 ± | 0.76 ± | 1.04 ± | 1.08 ± | 0.94 ± | 1.18 ± | 1.10 ± | 1.43 ± | 1.44 ± | 1.62 ± |
| | 0.34 | 0.31 | 0.33 | 0.35 | 0.33 | 0.33 | 0.32 | 0.32 | 0.32 | 0.32 | 0.33 |
| TRAF2 | 1.00 ± | 0.99 ± | 0.77 ± | 0.76 ± | 0.94 ± | 0.80 ± | 1.27 ± | 0.95 ± | 0.93 ± | 1.10 ± | 1.13 ± |
| | 0.29 | 0.27 | 0.27 | 0.28 | 0.27 | 0.29 | 0.29 | 0.27 | 0.26 | 0.27 | 0.26 |
| TSC1 | 1.00 ± | 0.98 ± | 0.97 ± | 1.20 ± | 1.39 ± | 1.08 ± | 1.10 ± | 1.12 ± | 1.12 ± | 1.18 ± | 1.18 ± |
| | 0.18 | 0.17 | 0.17 | 0.19 | 0.17 | 0.19 | 0.19 | 0.17 | 0.17 | 0.17 | 0.17 |
| WT1 | 1.00 ± | 0.78 ± | 0.76 ± | 1.34 ± | 1.26 ± | 0.98 ± | 1.05 ± | 0.91 ± | 0.80 ± | 0.92 ± | 1.04 ± |
| | 0.32 | 0.30 | 0.29 | 0.38 | 0.32 | 0.30 | 0.30 | 0.30 | 0.30 | 0.29 | 0.30 |
| XRCC5 | 1.00 ± | 1.03 ± | 0.91 ± | 0.92 ± | 1.00 ± | 0.98 ± | 1.19 ± | 1.11 ± | 1.12 ± | 0.97 ± | 1.10 ± |
| | 0.15 | 0.15 | 0.14 | 0.15 | 0.14 | 0.15 | 0.17 | 0.15 | 0.15 | 0.14 | 0.14 |
| ACTB | 1.00 ± | 0.97 ± | 0.96 ± | 0.69 ± | 0.88 ± | 0.86 ± | 0.83 ± | 1.00 ± | 1.03 ± | 0.83 ± | 0.87 ± |
| | 0.22 | 0.20 | 0.20 | 0.20 | 0.23 | 0.22 | 0.20 | 0.20 | 0.21 | 0.20 | 0.20 |
| B2M | 1.00 ± | 1.10 ± | 1.09 ± | 1.22 ± | 0.99 ± | 1.09 ± | 1.04 ± | 0.97 ± | 0.99 ± | 1.32 ± | 1.40 ± |
| | 0.20 | 0.18 | 0.19 | 0.19 | 0.20 | 0.22 | 0.19 | 0.19 | 0.21 | 0.19 | 0.19 |
| GAPDH | 1.00 ± | 0.89 ± | 0.87 ± | 0.95 ± | 1.12 ± | 0.93 ± | 0.91 ± | 0.90 ± | 1.02 ± | 0.84 ± | 0.81 ± |
| | 0.18 | 0.17 | 0.17 | 0.17 | 0.18 | 0.19 | 0.16 | 0.18 | 0.18 | 0.17 | 0.17 |
| HPRT1 | 1.00 ± | 1.03 ± | 0.96 ± | 0.85 ± | 0.77 ± | 0.89 ± | 0.98 ± | 0.89 ± | 0.79 ± | 0.83 ± | 0.85 ± |
| | 0.20 | 0.18 | 0.19 | 0.18 | 0.19 | 0.19 | 0.19 | 0.19 | 0.19 | 0.19 | 0.19 |
| RPLP0 | 1.00 ± | 0.96 ± | 1.08 ± | 1.38 ± | 1.36 ± | 1.28 ± | 1.23 ± | 1.24 ± | 1.19 ± | 1.24 ± | 1.12 ± |
| | 0.12 | 0.11 | 0.12 | 0.12 | 0.15 | 0.11 | 0.11 | 0.12 | 0.13 | 0.12 | 0.12 |

Plasma concentrations were in the expected range for both dose levels based on previous PK studies. Tumors harvested from mice treated with Compound 2 at 50 mg/kg QD×6 exhibited reductions in mutant p53 (39%) by 24 h post initial dose and at 8 h (47-75%) following each subsequent dose when compared to vehicle treated tumors. The reduction was accompanied by increases in WT conformation p53 (2.3 to 2.7-fold) 8 h after daily administrations when plasma concentrations of Compound 2 were highest (˜11,000 ng/mL). Tumors from mice administered 100 mg/kg Compound 2 QD×6 had a similar reduction at 24 h (42%) in mutant p53 as the 50 mg/kg group but greater increases in WT conformation p53 (2.3 to 3.5-fold) through the first 24 h of treatment. Enhanced reduction of mutant p53 (70-86%) and restoration of WT conformation p53 (2.6 to 3.7-fold) was seen 8 h post dose on days 2, 4, and 6 when Compound 2 exposure was approximately 18,600 ng/mL following repeat dosing at 100 mg/kg.

Analysis of the target proteins downstream of WT conformation p53 revealed increases in MDM2 (7.6 to 15.9-fold) and p21 (2.4 to 5.0-fold) that are consistent with Compound 2 plasma exposures ˜11,000 ng/mL measured at 8 h post-dose in tumors from mice treated with 50 mg/kg QD×6. The higher dose of 100 mg/kg QD×6 resulted in increased exposure (˜18,600 ng/mL) and as such increased MDM2 (10.6 to 32.6-fold) and p21 (3.9 to 6.5-fold) levels 8 h post dose. An increase in MIC-1 levels measured in the plasma was observed at 8 h (2.4-3.7 pg/mL/mm³) post-dose for mice that received 50 mg/kg Compound 2 QD×6. Mice that received 100 mg/kg Compound 2 QD×6 showed a proportional increase in MIC-1 (3.3-6.9 pg/mL/mm³) observed daily at 8 h post-dose and correlated with peak plasma exposure of Compound 2.

p53 target gene expression was also assessed with tumors on mice that received 50 mg/kg QD×6 dose of Compound 2 demonstrating increases in p21 (4.5 to 6.1-fold) and MDM2 (2.9 to 3.8-fold) at 8 h daily and a maximum 44% decrease in BIRC5, the gene that encodes the anti-apoptotic protein Survivin, at 8 h following the second dose. At the 100 mg/kg QD×6 dose of Compound 2 increases in p21 (7.1 to 11.9-fold) and MDM2 (4.2 to 5.4-fold) were measured 8 h post-dose daily and a maximum 77% decrease in BIRC5 at 24 h post the first dose. A larger panel of genes were assessed for the group of mice administered Compound 2 at 100 mg/kg QD×6. Robust increases in positively regulated genes were measured immediately post-dosing with p21 and MDM2 being the most highly increased p53-dependent mRNAs. Peak increases in p21 and MDM2 occurred daily at approximately 8 h post dose and ranged from a 5.8- to 9.3-fold and a 4.0- to 4.3-fold increase for p21 and MDM2, respectively. Modulations in gene expression changes correlated with peak plasma concentrations of Compound 2 at approximately 18,000 ng/mL 8 h post-dose. Positively regulated genes decreased in expression (although often not back to baseline) by 24 h as plasma compound levels decreased to ˜3,300 ng/mL. Negatively regulated genes showed decreases in expression that were greatest at 24 h post the first dose, the negatively regulated genes in general did not show a pattern of daily modulation rather they tended to stay slightly deceased across the course of study.

### Example 13: Measurement of the PK Response to Compound 2 in Naïve Balb/c Nude Mice Treated with 100 mg/kg (QD×1), 300 mg/kg (QD×1), or 300 mg/kg (BID×1)

The PK parameters of Compound 2 in naïve Balb/c nude female mice were measured. Mice were dosed orally (PO) with Compound 2 at 100 mg/kg or 300 mg/kg single dose (QD×1) or 300 mg/kg twice per day (BID×1, separated by 8 hours). Plasma was harvested at 1, 2, 4, 7, 24, 48, 72, 96, 120, and 144 hours (h) post dose for the 100 mg/kg and 300 mg/kg QD groups and 1, 2, 4, 7, 9, 12, 24, 48, 72, 96, 120, and 144 h post initial dose for the 300 mg/kg BID group. Animals were rotated between timepoints to form a composite PK curve. Plasma was isolated from blood by centrifugation at 18.8×g and frozen at −80° C. prior to analysis. Plasma was analyzed for Compound 2 levels by LC-MS/MS. TABLE 66 shows treatment groups and dosing regimens.

TABLE 66

| | | | | Dosing | | |
|---|---|---|---|---|---|---|
| | | | | Frequency & | Dose | Harvest timepoints |
| Group | Treatment | N | Route | Duration | (mg/kg) | (h post last dose) |
| 1 | Compound 2 | 12 | PO | QDx1 | 100 | 1, 2, 4, 7, 24, 48, 72, |
| | | (3/tp) | | | | 96, 120, 144 |
| 2 | Compound 2 | 12 | PO | QDx1 | 300 | 1, 2, 4, 7, 24, 48, 72, |
| | | (3/tp) | | | | 96, 120, 144 |
| 3 | Compound 2 | 12 | PO | BIDx1 (7.5 h) | 300 | 1, 2, 4, 7, 9, 12 24, 48, |
| | | (3/tp) | | | | 72, 96, 120, 144 |

Female Balb/c nude mice (150 total) were acclimatized as described in EXAMPLE 7. Naïve, female, 7-8-week Balb/c nude mice were randomly distributed into three groups (N=3 per cage). Bioanalysis: An aliquot of 20 μL sample was protein precipitated with 200 μL IS solution (100 ng/mL Labetalol, 100 ng/mL Tolbutamide, and 100 ng/mL Diclofenac in ACN), the mixture was vortexed and centrifuged at 4000 rpm for 15 min at 4° C. An 80 μL aliquot of the supernatant was transferred to the sample plate and mixed with 80 μL of water, then the plate was shaken at 800 rpm for 10 min. 0.5-4 μL supernatant was then injected into a Triple Quad 6500+ for LC-MS/MS analysis. The calibration curve was generated at 1-3000 ng/mL for the compounds in Balb/c nude Mouse Plasma (EDTA-K2) All values deemed below the level of quantification (BQL) were excluded from the PK parameters calculations. Following oral administration of Compound 2 at 100 mg/kg in female Balb/c nude mice, the average maximum plasma concentration (Cₘₐₓ) was 61,267 ng/mL, with a time to reach Cₘₐₓ (Tₘₐₓ) value of 1 h. The area under the plasma concentration-time curve from time zero to the last quantifiable concentration (AUC₀₋ₗₐₛₜ) was 763,547 ng·h/mL. The area under the plasma concentration-time curve from time zero to 24 hours (AUC₀₋₂₄) was 763,547 ng·h/mL. The average maximum plasma concentration at the last quantifiable concentration (Cₗₐₛₜ) was 2913 ng/mL, with a time to reach Cₗₐₛₜ (Tₗₐₛₜ) value of 24 hours. The area under the plasma concentration-time curve from time zero to infinity (AUC_{0-inf}) was calculated to be 782,504 ng·h/mL.

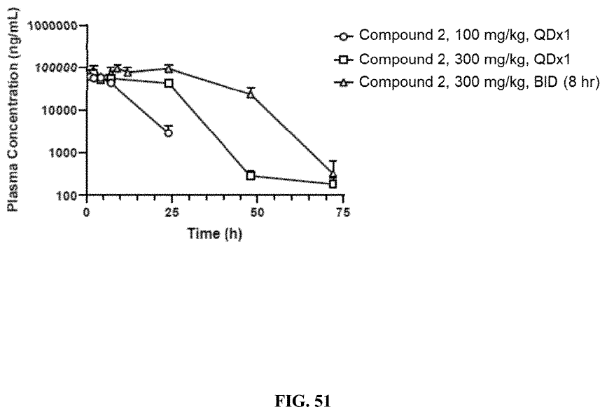

Following oral administration of Compound 2 at 300 mg/kg in female Balb/c nude mice, the average Cₘₐₓ was 72,767 ng/mL, with a Tₘₐₓ value of 1 h. The AUC₀₋ₗₐₛₜ was 1,773,056 ng·h/mL and AUC₀₋₂₄ was 1,244,100 ng·h/mL. The average Cₗₐₛₜ was 181 ng/mL, with a Tₗₐₛₜ value of 72 h. The average AUC_{0-inf} was calculated to be 1,774,963 ng·h/mL. Following oral administration of Compound 2 at 300 mg/kg, BID (8 h) in female Balb/c nude mice, the average Cₘₐₓ was 95,833 ng/mL, with a Tₘₐₓ value of 9 h. The AUC₀₋ₗₐₛₜ was 3,600,513 ng·h/mL with AUC₀₋₂₄ was 1,913,017 ng·h/mL. The average Cₗₐₛₜ was 325 ng/mL, with a Tₗₐₛₜ value of 72 h and the average AUC_{0-inf} was 3,603,269 ng·h/mL. TABLE 67 shows the PK response to Compound 2. FIG. 51 shows changes in plasma concentration (ng/mL) over time for mice treated with 100 mg/kg (QD×1), 300 mg/kg (QD×1), and 300 mg/kg (BID 8 hr) of Compound 2.

TABLE 67

| | 100 mg/kg | 300 mg/kg | 300 mg/kg |
|---|---|---|---|
| | QDx1 | QDx1 | BIDx1 (8 h) |
| Tₘₐₓ (h) | 1 | 1 | 9 |
| Cₘₐₓ (ng/mL) | 61267 | 72767 | 95833 |
| AUC₀₋ₗₐₛₜ (ng · h/mL) | 763547 | 1773056 | 3600513 |
| AUC₀₋₂₄ (ng · h/mL) | 763547 | 1244100 | 1913017 |
| Tₗₐₛₜ (h) | 24 | 72 | 72 |
| Cₗₐₛₜ (ng/mL) | 2913 | 181 | 325 |
| AUC_{0-inf} (ng · h/mL) | 782504 | 1774963 | 3603269 |

A single value is used for the calculation in Phoenix therefore standard deviation cannot be calculated.

Conclusion: Treatment with 100 mg/kg and 300 mg/kg Compound 2 QD resulted in non-dose proportional increases in Cₘₐₓ of 61,267 and 72,767 ng/mL, respectively. Mice treated with 300 mg/kg BID (8 h) Compound 2 displayed a Cₘₐₓ of 95,833 ng/mL at 9 h post-dose. The Tₘₐₓ for the single dosed groups of 100 and 300 mg/kg were both reached quickly by 1 h, while the Tₘₐₓ for the 300 mg/kg BID group was reached at 9 h (1 h post the second dose). The AUC₀₋ₗₐₛₜ for all groups showed a dose proportional increase in plasma exposure with increasing dose. Mice treated with Compound 2 at 100 mg/kg and 300 mg/kg QD had an average AUC₀₋ₗₐₛₜ of 763,547 and 1,773,056 ng·h/mL, respectively. The dosing regimen of 300 mg/kg BID (8 h) also resulted in a two-fold increase in AUC₀₋ₗₐₛₜ (3,600,513 ng·h/mL) compared to the 300 mg/kg single dose group (1,773,056 ng·h/mL). Mice treated with 100 mg/kg and 300 mg/kg Compound 2 exhibited dose dependent increases in exposure (Cₘₐₓ and AUC₀₋ₗₐₛₜ). The 300 mg/kg BID×1 group demonstrated proportionally improved exposure over the 300 mg/kg QD×1 group. All animals tolerated the compound well during the entire course of the study, and no adverse effects were observed.

### Example 14: Measurement of the PK Response to Compound 2 in Naïve Balb/c Nude Mice

The PK parameters of Compound 2 were tested in female naïve Balb/c nude mice. Mice were given a single oral dose (PO) of Compound 2 at 25 mg/kg, 50 mg/kg, or 100 mg/kg (QD×1). Plasma was harvested at 1, 2, 4, 7, and 24 hours (h) post dose via retro orbital sinus or cardiac puncture. Animals were rotated between timepoints to form a composite PK curve. Blood was collected into EDTA 1.5 mL blood collection tubes and centrifuged at 18.8×g to isolate plasma that was subsequently stored at −80° C. The samples were analyzed for Compound 2 levels by LC-MS/MS. TABLE 68 shows the treatment groups and corresponding dosing regimens for the study.

TABLE 68

| | | | | Dosing | | |
|---|---|---|---|---|---|---|
| | | | | Frequency & | Dose | Harvest timepoints |
| Group | Treatment | N | Route | Duration | (mg/kg) | (h post last dose) |
| 1 | Compound 2 | 6 | PO | QDx1 | 25 | 1, 2, 4, 7, 24 |
| | | (3/tp) | | | | |
| 2 | Compound 2 | 6 | PO | QDx1 | 50 | 1, 2, 4, 7, 24 |
| | | (3/tp) | | | | |
| 3 | Compound 2 | 6 | PO | QDx1 | 100 | 1, 2, 4, 7, 24 |
| | | (3/tp) | | | | |

Female Balb/c nude mice (180 total) were 8-10 weeks old at initiation of study and acclimatized according to EXAMPLE 7. The naïve, female, 7-8-week Balb/c nude mice were randomly distributed into three groups (N=3 per cage). Bioanalysis: An 20 μL aliquot of a sample was protein precipitated with 200 μL IS solution (100 ng/mL Labetalol, 100 ng/mL Tolbutamide, and 100 ng/mL Diclofenac in ACN), vortex-mixed, and spun by centrifuge at 4000 rpm for 15 min at 4° C. An 80 μL aliquot of the supernatant was transferred to the sample plate and mixed with 80 μL of water, then the plate was shaken at 800 rpm for 10 min. 0.5-4 μL of supernatant was then injected into a Triple Quad 6500+ for LC-MS/MS analysis. The calibration curve was generated at 1-3000 ng/mL for the compounds in Balb/c nude Mouse Plasma (EDTA-K2). PK parameters were calculated using Phoenix 64 software. All values deemed below the level of quantification (BQL) were excluded from the PK parameters calculations.

Following oral administration of Compound 2 at 25 mg/kg in female Balb/c nude mice, the average maximum plasma concentration (Cₘₐₓ) was 4,713 ng/mL, with a time to reach Cₘₐₓ (Tₘₐₓ) value of 2 h. The area under the plasma concentration-time curve from time zero to 24 hours (AUC₀₋₂₄) was 38,923 ng·h/mL. The average maximum plasma concentration at the last quantifiable concentration (Cₗₐₛₜ) was 16 ng/mL, with a time to reach Cₗₐₛₜ (Tₗₐₛₜ) value of 24 hours. The area under the plasma concentration-time curve from time zero to infinity (AUC_{0-inf}) was 38,984 ng·h/mL.

Oral administration of Compound 2 at 50 mg/kg in female Balb/c nude mice resulted in an average Cₘₐₓ of 9,683 ng/mL, with a Tₘₐₓ value of 2 h and an AUC₀₋₂₄ of 116,551 ng·h/mL. The average Cₗₐₛₜ was 746 ng/mL, with a Tₗₐₛₜ value of 24 h. The average AUC_{0-inf} was 122,764 ng·h/mL.

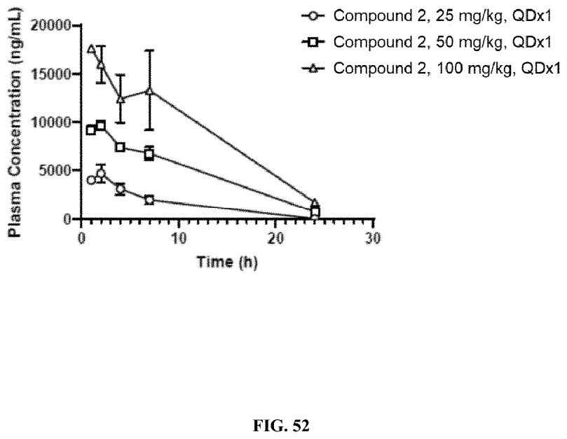

At the higher dose of 100 mg/kg Compound 2, QD×1 in female Balb/c nude mice, the average Cₘₐₓ was 17,633 ng/mL, with a Tₘₐₓ value of 1 h. The AUC₀₋₂₄ was 220,270 ng·h/mL while the average Cₗₐₛₜ was 1,720 ng/mL, with a Tₗₐₛₜ value of 24 h. AUC_{0-inf} was 236,987 ng·h/mL. TABLE 69 shows plasma concentrations over time for Compound 2 at three dose levels. FIG. 52 shows changes in plasma concentration (ng/mL) over time in mice treated with 25 mg/kg (QD×1), 50 mg/kg (QD×1), or 100 mg/kg (QD×1) of Compound 2.

TABLE 69

| | Compound 2 | Compound 2 | Compound 2 |
|---|---|---|---|
| | 25 mg/kg | 50 mg/kg | 100 mg/kg |
| | QDx1 | QDx1 | QDx1 |
| Tₘₐₓ (h) | 2 | 2 | 1 |
| Cₘₐₓ (ng/mL) | 4713 | 9683 | 17633 |
| AUC₀₋₂₄ (ng · h/mL) | 38923 | 116551 | 220270 |
| Tₗₐₛₜ (h) | 24 | 24 | 24 |
| Cₗₐₛₜ (ng/mL) | 16 | 746 | 1720 |
| AUC_{0-inf} (ng · h/mL) | 38984 | 122764 | 236987 |

The PK parameters of Compound 2 were determined at three dose levels. All animals tolerated the compound well during the entire course of the study, and no adverse effects were observed during the in-life phase of the study. Female nude mice administered Compound 2 QD×1 PO at 25 mg/kg, 50 mg/kg, or 100 mg/kg demonstrated a dose proportional increase in Cₘₐₓ of 4713, 9683, and 17633 ng/mL, respectively. The Tₘₐₓ was short for all dose levels between 1-2 h. The AUC₀₋₂₄ for across the dose levels also showed dose proportional increases in plasma exposure, mice administered with Compound 2 at 25 mg/kg, 50 mg/kg, and 100 mg/kg QD×1 had an average AUC₀₋₂₄ of 38,923, 116,551, and 220,270 ng·h/mL, respectively.

### Example 15: Measurement of the PK Response to Compound 2 in Sprague Dawley Female Rats

The PK parameters of Compound 2 were determined in female naïve Sprague Dawley Rats. Rats were given a single oral dose (PO) of Compound 2 at 25 mg/kg, 100 mg/kg, or 300 mg/kg (QD×1). Plasma was harvested at 1, 2, 4, 8, 24, 48, 72, and 96 hours (h) post dose. Blood was collected into EDTA 1.5-mL blood collection tubes and spun by centrifuge at 18.8*g for 2 min at 4° C. to isolate plasma that was subsequently stored at −80° C. The samples were analyzed for Compound 2 levels by LC-MS/MS. TABLE 70 shows the treatment groups and dosing regimens of the study.

TABLE 70

| | | | | Dosing | | |
|---|---|---|---|---|---|---|
| | | | | Frequency & | Dose | Harvest timepoints |
| Group | Treatment | N | Route | Duration | (mg/kg) | (h post last dose) |
| 1 | Compound 2 | 3 | PO | QDx1 | 25 | 1, 2, 4, 8, 24, 48, |
| | | (3/tp) | | | | 72 |
| 2 | Compound 2 | 3 | PO | QDx1 | 100 | 1, 2, 4, 8, 24, 48, |
| | | (3/tp) | | | | 72, 96* |
| 3 | Compound 2 | 3 | PO | QDx1 | 300 | 1, 2, 4, 8, 24, 48, |
| | | (3/tp) | | | | 72, 96* |

*96 h collected only for 100 and 300 mg/kg groups.

Female Sprague Dawley Rats (10 total) were 7-8 weeks old at initiation of study and acclimatized as described in EXAMPLE 7. The naïve, female, 7-8-week Sprague Dawley Rats were randomly distributed into three groups (N=1 or 2 per cage). The bioanalysis and PK analyses were performed as described in EXAMPLE 14. Following oral administration of Compound 2 at 25 mg/kg in female Sprague Dawley rats, the average maximum plasma concentration (Cₘₐₓ) was 6,647 ng/mL with a time to reach Cₘₐₓ (Tₘₐₓ) value of 6.7 h. The area under the plasma concentration-time curve from time zero to 24 hours (AUC₀₋₂₄) was 104,965 ng·h/mL with a half-life of 9.35 h. The area under the plasma concentration-time curve from time zero to Tₗₐₛₜ (AUC₀₋ₗₐₛₜ) and the area under plasma concentration-time curve from time zero extrapolated to infinity (AUC_{0-inf}) were 128,419 and 129,143 ng·h/mL, respectively.

Oral administration of Compound 2 at 100 mg/kg in female Sprague Dawley rats resulted in an average Cₘₐₓ of 17,567 ng/mL with a Tₘₐₓ value of 13.3 h. The AUC₀₋₂₄ was 344,887 ng·h/mL with a half-life of 7.56 h. The average AUC₀₋ₗₐₛₜ and the AUC_{0-inf} were 643,017 and 643,560 ng·h/mL, respectively.

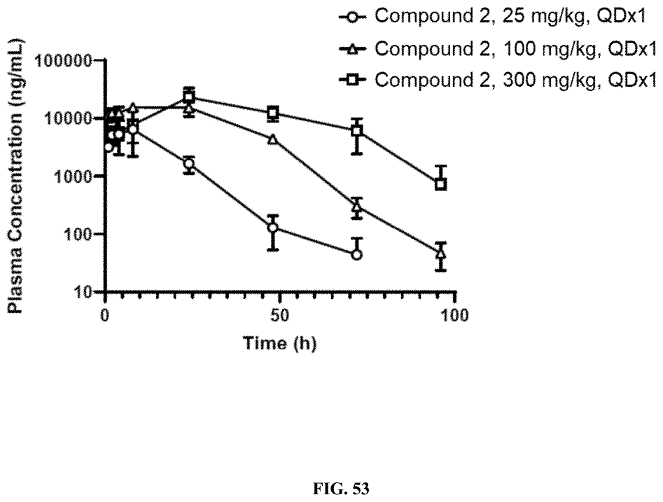

At the higher dose of 300 mg/kg Compound 2 in female Sprague Dawley rats, the average Cₘₐₓ was 23,033 ng/mL with a Tₘₐₓ value of 24 h. The AUC₀₋₂₄ was 301,758 ng·h/mL with a half-life of 11.43 h while the AUC₀₋ₗₐₛₜ and (AUC_{0-inf}) were 1,031,826 and 1,046,901 ng·h/mL, respectively. TABLE 71 shows plasma concentrations over time for Compound 2 at three dose levels. FIG. 53 shows changes in plasma concentration (ng/mL) for mice treated with 25 mg/kg (QD×1), 100 mg/kg (QD×1), or 300 mg/kg (QD×1).

TABLE 71

| | Compound 2 | Compound 2 | Compound 2 |
|---|---|---|---|
| | 25 mg/kg QDx1 | 100 mg/kg QDx1 | 300 mg/kg QDx1 |
| Tₘₐₓ (h) | 6.67 ± 2.31 | 13.33 ± 9.24 | 24.00 ± 0.00 |
| Cₘₐₓ (ng/mL) | 6,647 ± 2,424 | 17,567 ± 2969 | 23,033 ± 10,027 |
| AUC₀₋₂₄ (ng · h/mL) | 104,965 ± 34,757 | 344,887 ± 51,248 | 301,758 ± 139,546 |
| Half-life (h) | 9.35 ± 3.34 | 7.56 ± 1.41 | 11.43 ± 4.30 |
| AUC₀₋ₗₐₛₜ (ng · h/mL) | 128,419 ± 38,306 | 643,017 ± 105,298 | 1,031,826 ± 134,153 |
| AUC_{0-inf} (ng · h/mL) | 129,143 ± 38,151 | 643,560 ± 104,937 | 1,046,901 ± 139,695 |

The PK parameters of Compound 2 were determined at three dose levels. All animals tolerated the compound well during the course of study, and no adverse effects were observed during the in-life phase of the study. Female Sprague Dawley rats administered Compound 2 PO at 25 mg/kg, 100 mg/kg, or 300 mg/kg resulted in increases in Cₘₐₓ of 6647, 17566, and 23033 ng/mL, respectively, with increasing doses. The Tₘₐₓ values increased with increased doses and was between 6-24 h. The oral half-lives of Compound 2 were similar at the 25 mg/kg and 100 mg/kg dose levels (8-9 h), while the half-lives increased at the higher doses of 300 mg/kg to ˜ 11 h. The AUC₀₋ₗₐₛₜ and AUC_{0-inf} values both increased across dose levels and appeared dose-proportional between the 25 mg/kg and 100 mg/kg levels. However, the values were less than dose proportional between 100 mg/kg and 300 mg/kg. Rats administered with Compound 2 at 25 mg/kg, 100 mg/kg, and 300 mg/kg QD×1 had average AUC₀₋ₗₐₛₜ values of 128,419, 643,017, and 1,031,826 ng·h/mL, respectively, and AUC_{0-inf} values of 129,143, 643,560, and 1,046,901 ng·h/mL, respectively.

### Example 16: PK of Compound 2 Following IV and Oral Administration in Female Sprague-Dawley Rats

The PK of Compound 2 was determined following a single IV and oral (PO) administration at different dose levels in female Sprague-Dawley rats. Nine female Sprague-Dawley rats were randomly assigned to three equal groups; one group was administered Compound 2 by tail vein injection at 2.5 mg/kg while the other two were given the compound by oral gavage at 50 mg/kg and 300 mg/kg. For the IV route, Compound 2 was dissolved in 40% hydroxypropyl-beta-cyclodextrin (HPβCD) in water at 1 mg/mL and administered at 2.5 mL/kg. For the PO route, Compound 2 was formulated in 2% hydroxypropyl cellulose (HPC) and 0.5% Tween 80 in water at 10 and 60 mg/mL and administered at 5 mL/kg for the 50 mg/kg and 300 mg/kg dose, respectively. Blood samples were collected serially at 0.083, 0.25, 0.5, 1, 2, 4, 8, and 24 hours following IV administration and at 0.5, 1, 2, 4, 8, 24, 48, 72, 96, 120, and 144 hours following PO dosing. The blood samples were processed for plasma by centrifugation and were stored at −70° C. until bioanalytical assay. The concentrations of Compound 2 in the plasma were determined by LC-MS/MS. The bioanalytical assay for Compound 2 had a LLOQ of 1 ng/mL and a linear range up to 3000 ng/mL. A non-compartmental analysis model was employed for the calculation of PK parameters. TABLE 72 shows formulation and dosing information. TABLE 73 shows blood sample collection information.

TABLE 72

| | | Dose | Dose | Dose | | |
|---|---|---|---|---|---|---|
| | | Level | Conc. | Volume | | Dosing |
| Group | n | (mg/kg) | (mg/mL) | (mL/kg) | Vehicle | Route |
| 4 | 3 | 2.5 | 1 | 2.5 | 40% HPβCD in | IV |
| | | | | | H₂O | |
| 5 | 3 | 50 | 10 | 5 | 2% HPC, 0.5% | PO |
| | | | | | Tween 80 in H₂O | |
| 6 | 3 | 300 | 60 | 5 | 2% HPC, 0.5% | PO |
| | | | | | Tween 80 in H₂O | |

TABLE 73

| Group | Matric | Sampling schedule post dosing (h) |
|---|---|---|
| 4 | Plasma | 0.083, 0.25, 0.5, 1, 2, 4, 8, 24 |
| 5 | Plasma | 0.5, 1, 2, 4, 8, 24, 48, 72, 96, 120, 144 |
| 6 | Plasma | 0.5, 1, 2, 4, 8, 24, 48, 72, 96, 120, 144 |

Anticoagulant for blood: K₂EDTA.

Animals: The nine female Sprague-Dawley rats were group housed during acclimation and throughout the study under controlled temperature (20-26° C.), humidity (30-70%), and lights (12 h dark/light cycle). The animals were fed certified pellet diet and had access to water (reverse osmosis) ad libitum. All mice were confirmed healthy prior to being assigned to the study. Each mouse was given a unique identification number, which was marked on the tail and written on the cage card as well. The animals were not fasted prior to compound administration, and food and water were present the entire time during the study. Rats were weighed immediately prior to dosing. The administered volume was verified by weighing the loaded and unloaded syringe before and after dosing, respectively. The weight difference (g) served as confirmation of the amount (mL) of dose solution dispensed.

Blood Collection and Plasma Preparation: Approximately 100 μL of blood was collected at each scheduled time point from jugular veins. The allowed deviations of collection time from the nominal time were less than 1 min for collections taking place prior to or at 1 hour, or less than 5% of the nominal values for collections taking place beyond 1 hour. The blood samples were placed in labeled micro-centrifuge tubes pre-treated with K₂EDTA as anticoagulant. Plasma samples were prepared by centrifuging the blood samples at approximately 4° C., 3000 g for 15 minutes. The plasma samples were then quickly frozen on dry ice and stored at −70±10° C. until LC-MS/MS analysis.

Dose Concentration Verification: The concentrations of Compound 2 in the dose solutions were determined by LC-MS/MS to verify the dose accuracy. The measured concentrations of Compound 2 were 0.987 mg/mL in the IV formulation and 9.81 and 56.3 mg/mL in the low and high dose PO formulations, respectively (TABLE 74). Compared to the corresponding nominal concentrations of 1, 10, and 60 mg/mL, the dose accuracy for IV route and the two PO routes was 98.7, 98.1, and 93.8%, respectively.

TABLE 74

| | | Measured Conc. | | | | |
|---|---|---|---|---|---|---|
| | | (mg/mL) | | Mean | Nominal | Accu- |
| | Dose | Sample | Sample | Conc. | Conc. | racy^{a} |
| Group | (mg/kg) | 1 | 2 | (mg/mL) | (mg/mL) | (%) |
| 4 | 2.5 | 1.02 | 0.954 | 0.987 | 1 | 98.7 |
| 5 | 50 | 9.78 | 9.84 | 9.81 | 10 | 98.1 |
| 6 | 300 | 61.8 | 50.7 | 56.3 | 60 | 93.8 |

^{a}Accuracy (%) = Mean Concentration (mg/mL)/Nominal Concentration (mg/mL) × 100.

PK analysis: A non-compartmental PK model and linear/log trapezoidal method were employed for PK calculation. The plasma concentrations below the LLOQ prior to Tₘₐₓ were set to zero, and those after Tₘₐₓ were excluded from the PK calculation. Nominal dose levels and nominal sample collection times were applied to the PK calculation. The values of plasma concentrations and PK parameters were reported in three significant figures. The average values of each dose group were presented as mean±SD.

TABLE 75 shows plasma concentrations (ng/mL) of Compound 2 following an IV administration at 2.5 mg/kg in female Sprague-Dawley rats. TABLE 76 shows plasma concentrations (ng/mL) of Compound 2 following an oral administration at 50 mg/kg in female Sprague-Dawley rats. TABLE 77 shows plasma concentrations (ng/mL) of Compound 2 following an oral administration at 300 mg/kg in female Sprague-Dawley rats. TABLE 78 shows PK parameters of Compound 2 following an IV administration at 2.5 mg/kg in female Sprague-Dawley rats. TABLE 79 shows PK parameters of Compound 2 following an oral administration at 50 mg/kg in female Sprague-Dawley rats. TABLE 80 shows PK parameters of Compound 2 following an oral administration at 300 mg/kg in female Sprague-Dawley rats.

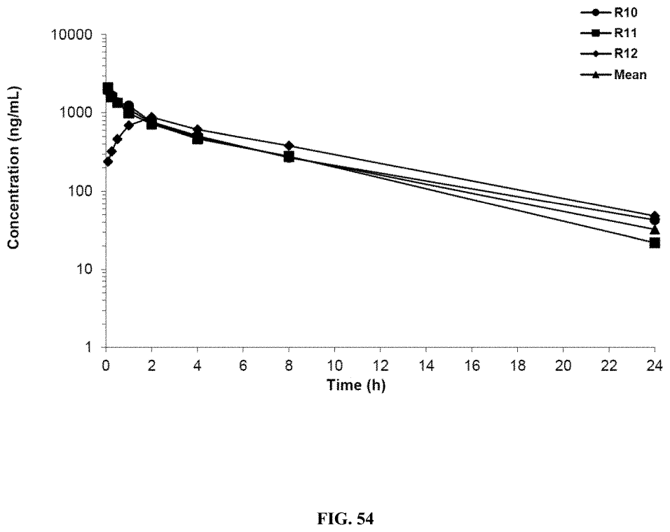

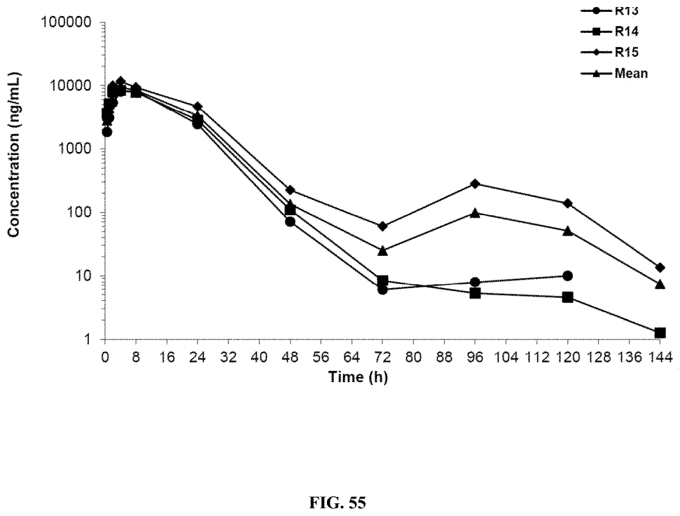

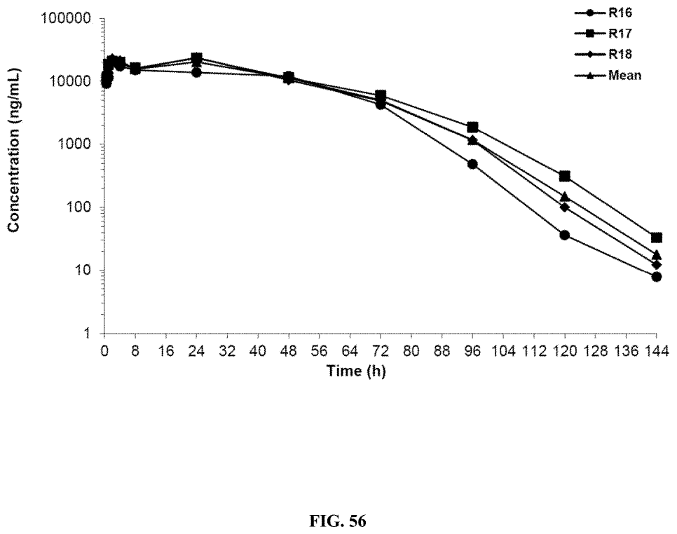

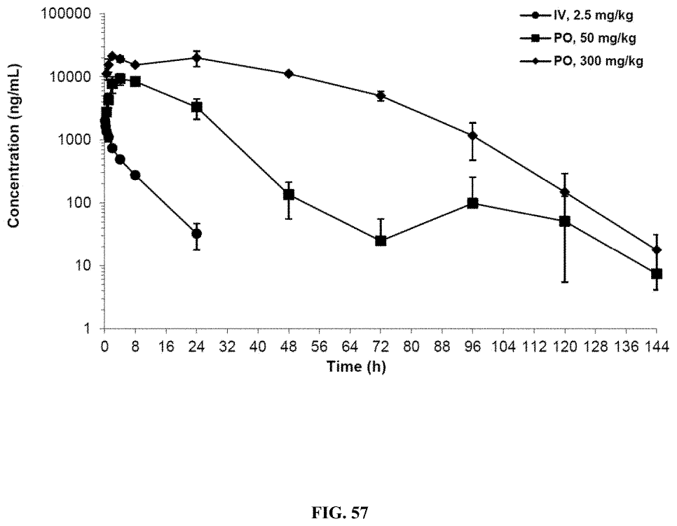

The plasma concentration-time profiles of the test article are illustrated in FIG. 54-FIG. 57, respectively. FIG. 54 shows individual and mean plasma concentration-time profiles of Compound 2 following an IV administration at 2.5 mg/kg in female Sprague-Dawley rats. FIG. 55 shows individual and mean plasma concentration-time profiles of Compound 2 following an oral administration at 50 mg/kg in female Sprague-Dawley rats. FIG. 56 shows individual and mean plasma concentration-time profiles of Compound 2 following an oral administration at 300 mg/kg in female Sprague-Dawley rats. FIG. 57 shows a comparison of plasma concentration of Compound 2 following IV and PO administration in female Sprague-Dawley rats.

IV administration of Compound 2 at 2.5 mg/mL resulted in an apparent volume of distribution (Vdₛₛ) of 2.24±0.0916 L/kg and an area under the concentration-time curve (AUC₀₋ₗₐₛₜ) of 6888±467 ng·h/mL. The apparent total plasma clearance (CL) was 5.86±0.508 mL/min/kg. The terminal elimination half-life (Tin) and the mean residence time (MRT₀₋ₗₐₛₜ) were 5.07±0.917 hours and 5.54±0.310 hours, respectively.

Oral administration of Compound 2 at 50 mg/kg and 300 mg/kg yielded peak plasma concentrations (Cₘₐₓ) of 9410±1898 ng/mL and 22567±1305 ng/mL, respectively. The time at which the Cₘₐₓ were obtained (Tₘₐₓ) were 5.33±2.31 hours and 16.7±12.7 hours, respectively. The corresponding AUC₀₋ₗₐₛₜ were 177623±44918 ng·h/mL and 1048230±139767 ng·h/mL, the Tin values were 9.20±2.85 hours and 7.77±0.475 hours, and the MRT₀₋ₗₐₛₜ values were 14.9±2.95 and 33.7±1.41 hours, respectively. The apparent absolute oral bioavailability was greater than 100% for both the 50 mg/kg and 300 mg/kg doses.

Compound 2 exhibited an IV T_{1/2} of 5 h, a low Vdₛₛ of 2.24 L/kg and a low CL of 5.86 mL/min/kg. The oral doses of 50 mg/kg and 300 mg/kg resulted in less than dose proportional Cₘₐₓ values of 9410 ng/mL and 22567 ng/mL, respectively, but dose proportional AUC₀₋ₗₐₛₜ of 177623 ng·h/mL and 1048230 ng·h/mL, respectively. Both oral doses resulted in greater than 100% bioavailability, which is likely due to the significant difference in dose levels between IV and PO administrations. Compound 2 was well tolerated by all study animals at the administered doses. No adverse effects were observed throughout the study.

TABLE 75

| | Compound 2, 2.5 mg/kg (IV) | | | | | |
|---|---|---|---|---|---|---|
| | Plasma concentration (ng/mL) | | | | | |
| Time (h) | R10 | R11 | R12* | Mean | SD | CV (%) |
| 0.0830 | 1930 | 2100 | 240 | 2015 | ND | ND |
| 0.250 | 1690 | 1590 | 324 | 1640 | ND | ND |
| 0.500 | 1370 | 1350 | 464 | 1360 | ND | ND |
| 1.00 | 1230 | 991 | 690 | 1111 | ND | ND |
| 2.00 | 764 | 720 | 879 | 742 | ND | ND |
| 4.00 | 510 | 471 | 614 | 491 | ND | ND |
| 8.00 | 270 | 281 | 382 | 276 | ND | ND |
| 24.0 | 42.9 | 21.9 | 48.3 | 32.4 | ND | ND |

R#: rat ID.
*Data of Rat #12 was excluded from calculation of group means due to a failure of IV dosing.
ND: not determined due to insufficient number of values.

TABLE 76

| | Compound 2, 50 mg/kg (PO) | | | | | |
|---|---|---|---|---|---|---|
| | Plasma concentration (ng/mL) | | | | | |
| Time (h) | R13 | R14 | R15 | Mean | SD | CV (%) |
| 0.500 | 1830 | 3630 | 2840 | 2767 | 902 | 32.6 |
| 1.00 | 3060 | 5110 | 4820 | 4330 | 1109 | 25.6 |
| 2.00 | 5370 | 8130 | 9970 | 7823 | 2315 | 29.6 |
| 4.00 | 8050 | 8390 | 11600 | 9347 | 1959 | 21.0 |
| 8.00 | 8240 | 7840 | 9350 | 8477 | 782 | 9.23 |
| 24.0 | 2440 | 2830 | 4670 | 3313 | 1191 | 35.9 |
| 48.0 | 71.0 | 110 | 224 | 135 | 79.5 | 58.9 |
| 72.0 | 5.99 | 8.40 | 60.1 | 24.8 | 30.6 | 123 |
| 96.0 | 7.87 | 5.25 | 282 | 98.4 | 159 | 162 |
| 120 | 10.0 | 4.51 | 139 | 51.2 | 76.1 | 149 |
| 144 | BQL | 1.26 | 13.5 | 7.38 | ND | ND |

R#: rat ID.
ND: not determined due to insufficient number of values.

TABLE 77

| | Compound 2, 300 mg/kg (PO) | | | | | |
|---|---|---|---|---|---|---|
| | Plasma concentration (ng/mL) | | | | | |
| Time (h) | R16 | R17 | R18 | Mean | SD | CV (%) |
| 0.500 | 9170 | 10800 | 13700 | 11223 | 2294 | 20.4 |
| 1.00 | 11400 | 18400 | 17000 | 15600 | 3704 | 23.7 |
| 2.00 | 21100 | 20500 | 22600 | 21400 | 1082 | 5.05 |
| 4.00 | 17200 | 19800 | 21100 | 19367 | 1986 | 10.3 |
| 8.00 | 15000 | 16100 | 15500 | 15533 | 551 | 3.55 |
| 24.0 | 13800 | 23000 | 23600 | 20133 | 5493 | 27.3 |
| 48.0 | 12000 | 11300 | 10400 | 11233 | 802 | 7.14 |
| 72.0 | 4250 | 5940 | 4890 | 5027 | 853 | 17.0 |
| 96.0 | 482 | 1870 | 1160 | 1171 | 694 | 59.3 |
| 120 | 36.2 | 310 | 100 | 149 | 143 | 96.3 |
| 144 | 7.79 | 33.2 | 12.2 | 17.7 | 13.6 | 76.6 |

R#: rat ID.

TABLE 78

| Parameter | R10 | R11 | R12* | Mean | SD | CV (%) |
|---|---|---|---|---|---|---|
| Rsq_adj | 0.993 | 0.998 | 1.000 | 0.996 | ND | ND |
| C₀ (ng/mL) | 2062 | 2411 | 240 | 2237 | ND | ND |
| T_{1/2} (h) | 5.72 | 4.42 | 5.43 | 5.07 | ND | ND |
| Vdₛₛ (L/kg) | 2.31 | 2.18 | 2.75 | 2.24 | ND | ND |
| C_{L} (mL/min/kg) | 5.50 | 6.22 | 5.46 | 5.86 | ND | ND |
| Tₗₐₛₜ (h) | 24.0 | 24.0 | 24.0 | 24.0 | ND | ND |
| AUC₀₋ₗₐₛₜ (ng · h/mL) | 7218 | 6558 | 7253 | 6888 | ND | ND |
| AUC_{0-inf} (ng · h/mL) | 7572 | 6698 | 7631 | 7135 | ND | ND |
| MRT₀₋ₗₐₛₜ (h) | 5.76 | 5.32 | 7.16 | 5.54 | ND | ND |
| MRT_{0-inf} (h) | 6.99 | 5.84 | 8.38 | 6.42 | ND | ND |

R#: rat ID.
*Data of Rat #12 was excluded from calculation of group means due to a failure of IV dosing.
ND: not determined due to insufficient number of values.

TABLE 79

| Parameter | R13 | R14 | R15 | Mean | SD | CV (%) |
|---|---|---|---|---|---|---|
| Rsq_adj | 0.985 | 0.873 | 0.826 | 0.895 | 0.0817 | 9.12 |
| Cₘₐₓ (ng/mL) | 8240 | 8390 | 11600 | 9410 | 1898 | 20.2 |
| Tₘₐₓ (h) | 8.00 | 4.00 | 4.00 | 5.33 | 2.31 | 43.3 |
| T_{1/2} (h) | 5.90 | 10.7 | 10.9 | 9.20 | 2.85 | 31.0 |
| Tₗₐₛₜ (h) | 120 | 144 | 144 | 136 | 13.9 | 10.2 |
| AUC₀₋ₗₐₛₜ (ng · h/mL) | 145232 | 158737 | 228900 | 177623 | 44918 | 25.3 |
| AUC_{0-inf} (ng · h/mL) | 145317 | 158756 | 229114 | 177729 | 45005 | 25.3 |
| MRT₀₋ₗₐₛₜ (h) | 13.1 | 13.3 | 18.3 | 14.9 | 2.95 | 19.8 |
| MRT_{0-inf} (h) | 13.2 | 13.3 | 18.4 | 15.0 | 3.00 | 20.0 |
| Bioavailability (%) | — | — | — | 125 | — | — |

R#: rat ID.

TABLE 80

| Parameter | R16 | R17 | R18 | Mean | SD | CV (%) |
|---|---|---|---|---|---|---|
| Rsq_adj | 0.986 | 0.992 | 0.996 | 0.992 | 0.00484 | 0.489 |
| Cₘₐₓ (ng/mL) | 21100 | 23000 | 23600 | 22567 | 1305 | 5.78 |
| Tₘₐₓ (h) | 2.00 | 24.0 | 24.0 | 16.7 | 12.7 | 76.2 |
| T_{1/2} (h) | 7.74 | 8.25 | 7.30 | 7.77 | 0.475 | 6.12 |
| Tₗₐₛₜ (h) | 144 | 144 | 144 | 144 | 0.00 | 0.0 |
| AUC₀₋ₗₐₛₜ (ng · h/mL) | 890830 | 1157813 | 1096048 | 1048230 | 139767 | 13.3 |
| AUC_{0-inf} (ng · h/mL) | 890917 | 1158209 | 1096176 | 1048434 | 139895 | 13.3 |
| MRT₀₋ₗₐₛₜ (h) | 33.0 | 35.3 | 32.8 | 33.7 | 1.41 | 4.18 |
| MRT_{0-inf} (h) | 33.0 | 35.4 | 32.8 | 33.7 | 1.43 | 4.23 |
| Bioavailability (%) | — | — | — | 122 | — | — |

R#: rat ID.

Conclusion: Compound 2 exhibited a T_{1/2} and MRT₀₋ₗₐₛₜ of approximately 5 h and a relatively low Vdₛₛ and CL following IV administration. PO administration of Compound 2 exhibited longer Tin and MRT₀₋ₗₐₛₜ compared to IV dosing. The T_{1/2} following oral administration at two dose levels were comparable. However, the MRT₀₋ₗₐₛₜ for the 300 mg/kg dose was nearly doubled relatively to the 50 mg/kg dose. The oral doses of 50 and 300 mg/kg resulted in less than dose proportional Cₘₐₓ values but dose proportional AUC₀₋ₗₐₛₜ. The total oral bioavailability were nearly the same given a 6-fold difference in dose levels. The greater than 100% bioavailability is likely due to the significant difference in dose levels between IV and PO administrations. TABLE 81 shows a summary of PK parameters based on individual plasma concentration-time curves.

TABLE 81

| IV dose | | Oral dose | | |
|---|---|---|---|---|
| | 2.5 mg/kg | | 50 mg/kg | 300 mg/kg |
| C₀ (ng/mL) | 2237 ± 247 | C₀ (ng/mL) | 9410 ± 1898 | 22567 ± 1305 |
| T_{1/2} (h) | 5.07 ± 0.917 | Tₘₐₓ (h) | 5.33 ± 2.31 | 16.7 ± 12.7 |
| Vdₛₛ (L/kg) | 2.24 ± 0.0916 | T_{1/2} (h) | 9.20 ± 2.85 | 7.77 ± 0.475 |
| C_{L} (ng/mL/kg) | 5.86 ± 0.508 | AUC₀₋ₗₐₛₜ (ng · h/mL) | 177623 ± 44918 | 1048230 ± 139767 |
| AUC₀₋ₗₐₛₜ (ng · h/mL) | 6888 ± 467 | MRT₀₋ₗₐₛₜ (h) | 14.9 ± 2.95 | 33.7 ± 1.41 |
| MRT₀₋ₗₐₛₜ (h) | 5.54 ± 0.310 | Bioavailability (%) | 125 | 122 |

### Example 17: Plasma PK Study of Compound 2 Following Single IV Bolus and Oral Administrations to Non-Naïve Male Beagle Dogs

The PK properties of Compound 2 following single IV bolus and PO administrations of Compound 2 in male beagle dogs were studied. Three non-naïve beagle dogs were assigned to the study with a three-phase crossover design. In Phase 1, animals were administered Compound 2 by single IV bolus administration at 2.5 mg/kg. In Phases 2 and 3, animals were administered Compound 2 by single PO administration at 25 mg/kg and 100 mg/kg, respectively. Blood samples were collected at 0.083 hours (IV only), 0.25 h, 0.5 h, 1 h, 2 h, 4 h, 8 h, 12 h, 24 h, 48 h (PO only), and 72 h post-dose for Phases 1, 2, and 3. Clinical chemistry and hematology samples were collected at pre-dose (0 h) and 24 h post-dose. Concentrations of Compound 2 in plasma samples were determined by LC/MS/MS. TABLE 82 shows dosing and sampling regimens. TABLE 83 shows sampling time points of the three treatment groups.

TABLE 82

| | | | Dose | Target | | |
|---|---|---|---|---|---|---|
| | N | Dose | Volume | Dose Conc. | | Dosing |
| Group¹ | (gender) | (mg/kg) | (mL/kg) | (mg/mL) | Vehicle | Route |
| 1 | 3 Male | 2.5 | 2.5 | 1 | 2% Dimethylacetamide | IV |
| | | | | | (DMA), 20% PEG400, and | bolus |
| | | | | | 23% HPβCD w/v in H₂O | |
| 2 | 3 Male | 25 | 5 | 5 | 2% Hydroxypropyl cellulose, | PO² |
| | | | | | 0.5% Polysorbate 80 v/v in H₂O | |
| 3 | 3 Male | 100 | 5 | 20 | 2% Hydroxypropyl cellulose, | PO² |
| | | | | | 0.5% Polysorbate 80 v/v in H₂O | |

¹Cross-over design, 3 days washout between dosing.
²For oral dosing, the animals were fasted overnight, food was returned at 4 hours post-dose.

TABLE 83

| | Dose | Animal | Sampling time point (h) ¹ | | | | | | | | | | |
|---|---|---|---|---|---|---|---|---|---|---|---|---|---|
| Group | (mg/kg) | No. | 0.083 | 0.25 | 0.5 | 1 | 2 | 4 | 8 | 12 | 24 | 48 | 72 |
| 1 | 2.5 | D1001, | P² | P | P | P | P | P | P | P | P | —³ | P |
| | | D1002, | | | | | | | | | | | |
| | | D1003 | | | | | | | | | | | |
| 2 | 25 | D1001, | — | P | P | P | P | P | P | P | P | P | P |
| | | D1002, | | | | | | | | | | | |
| | | D1003 | | | | | | | | | | | |
| 3 | 100 | D1001, | — | P | P | P | P | P | P | P | P | P | P |
| | | D1002, | | | | | | | | | | | |
| | | D1003 | | | | | | | | | | | |

¹ Blood collection for clinical chemistry and hematology was sampled at pre-dose (0 h) and 24 hours post-dose.
²P = plasma.
³“—” means no sample collection.

Formulations: The IV vehicle for Phase I was 2% DMA, 20% PEG400, and 23% HPβCD w/v in water at 2.5 mg/mL. The PO vehicle for Phase 2 and 3 was 2% Hydroxypropyl cellulose, 0.5% Polysorbate 80 v/v in water at 5 mg/mL and 20 mg/mL. All dosing solutions were analyzed for Compound 2 concentration by a LC/UV method. Two aliquots were taken from the middle region of the IV dosing solution. Two aliquots were taken from the bottom, middle, and top regions of the PO dosing formulation. All formulation samples were stored at 2-8° C. until analysis by LC/UV. The measured concentrations of test article in each dose formulation were within 80% to 120% of the nominal concentrations.

Animals: The animal room environment was controlled and monitored for temperature (18-26° C.) and relative humidity (40-70%) with 10 to 20 air changes/hour. The room was on a 12-hour light/dark cycle except when interruptions are necessitated by study activities. Any temperature excursion from the targeted mean range of 18-26° C., was documented as a deviation. Any relative humidity excursion from the targeted mean range of 40-70% for more than 3 hours was documented as a deviation. Fresh drinking water was available to all animals ad libitum. Animals were fed twice daily. Stock dogs were fed approximately 220 grams of Certified Dog Diet daily. These amounts were adjusted as necessary based on food consumption of the group or an individual body weight change. For PO dose phase, animals were fed the afternoon (at 3:30 to 4:00 pm) prior to the day of oral dosing and the remaining food was removed at 7:00 pm. Food was withheld until 4-hour post-dose unless specified in this protocol. Animals were fed approximately 220 grams once on the day of dosing. For IV dose phase, animals were fed the same as daily diet. Cage-side animal observations for appearance and general health condition were performed before and after dosing as well as at each time point of sample collection.

Dose administration: Animals were weighed prior to dose administration on each day of dosing to calculate the actual dose volume. The body weights were in the range from 7.03 to 8.86 kg for males on the first dosing day. All animals in Phase 1 received a single IV bolus administration of Compound 2. The animals in Phases 2 and 3 received a single oral gavage administration of Compound 2. Actual dosing concentrations of all formulations were within ±20% of the nominal target dosing levels by LC/UV, as shown in TABLE 84.

TABLE 84

| | | | | | Measured | Calculated | Mean | Nominal | |
|---|---|---|---|---|---|---|---|---|---|
| Phase | Dose | Sample | | Dilution | Conc. | Conc. | Conc. | Conc. | Accuracy ^{a} |
| No. | Route | No. | Determination | Factor | (μg/mL) | (mg/mL) | (mg/mL) | (mg/mL) | (%) |
| 1 | [Pre] | Middle 1 | 1.00 | 20.0 | 52.1 | 1.04 | 1.03 | 1.00 | 103 |
| | IV | | 2.00 | | 51.8 | 1.04 | | | |
| | bolus | Middle 2 | 1.00 | | 50.5 | 1.01 | | | |
| | | | 2.00 | | 51.6 | 1.03 | | | |
| 2 | [Pre] | Top | 1.00 | 50.0 | 109 | 5.45 | 5.32 | 5.00 | 106 |
| | PO | | 2.00 | | 109 | 5.45 | | | |
| | | Middle | 1.00 | | 106 | 5.32 | | | |
| | | | 2.00 | | 107 | 5.33 | | | |
| | | Bottom | 1.00 | | 104 | 5.21 | | | |
| | | | 2.00 | | 104 | 5.20 | | | |
| 3 | [Pre] | Top | 1.00 | 100 | 51.8 | 5.18 | 5.25 | 5.00 | 105 |
| | PO | | 2.00 | | 51.9 | 5.19 | | | |
| | | Middle | 1.00 | | 55.1 | 5.51 | | | |
| | | | 2.00 | | 55.1 | 5.51 | | | |
| | | Bottom | 1.00 | | 50.5 | 5.05 | | | |
| | | | 2.00 | | 50.5 | 5.05 | | | |

^{a} Accuracy (%) = Mean Concentration (mg/mL)/Nominal Concentration (mg/mL) × 100.

Collection and preparation of plasma samples for PK analysis: Approximately 0.5 mL blood was collected at each time point via peripheral vessel from each study animal. The actual time for each sample collection was recorded. Deviations on sampling time were less than 1 minute for the time points pre-dose through 1 hour post-dose, and less than 5% of the nominal time for time points after 1 hour post-dose. All blood samples were transferred into commercial collection tubes containing K₂EDTA (0.85-1.15 mg). Plasma samples were then prepared by centrifuging the blood samples at approximately 2 to 8° C., 3000× g for 10 minutes. All plasma samples were then quickly frozen over dry ice and kept at −60° C. or lower until LC/MS/MS analysis.

Serum and blood samples for Clinical Chemistry analysis: For serum samples, whole blood samples (approximately 1.0 mL) without anticoagulant were collected and held at room temperature (RT) and up-right for at least 30 minutes. For blood samples, whole blood (at least 1.0 mL) was collected from the experimental animals into commercially available tubes with Potassium (K₂) EDTA at RT.

Data analysis: The concentrations of Compound 2 in plasma were determined by using a LC/MS/MS method. The plasma concentration of Compound 2 in study animals was subjected to a non-compartmental PK analysis. The linear/log trapezoidal rule was applied in obtaining the PK parameters. Individual plasma concentration values that were below the LLOQ were excluded from the PK parameter calculation. All plasma concentrations and PK parameters were reported with three significant figures. The nominal dose levels and nominal sampling times were used in the calculation of all PK parameters.

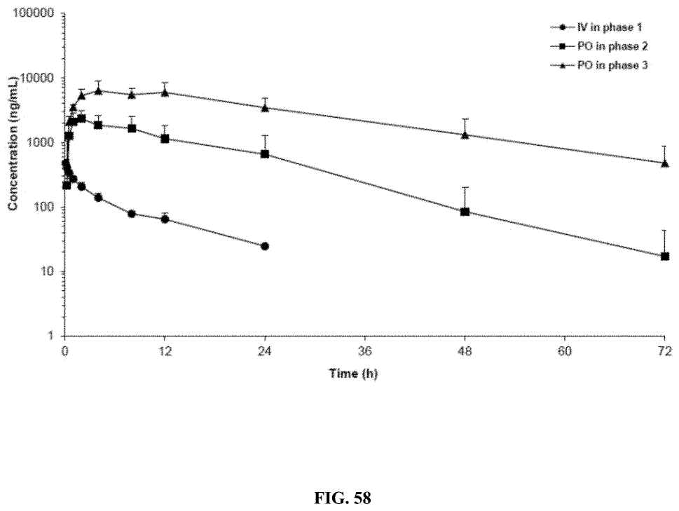

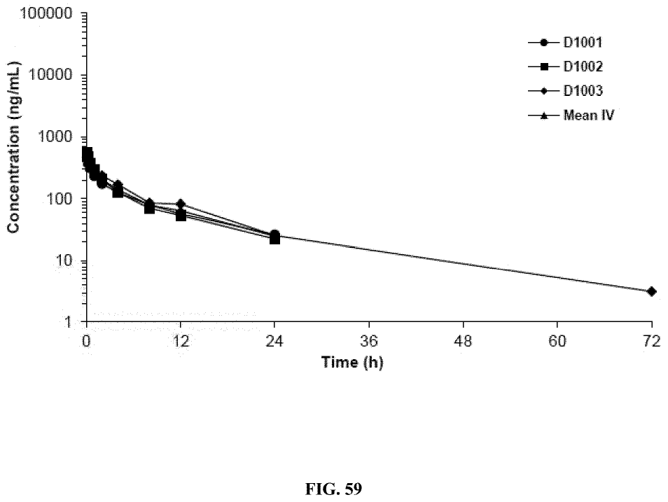

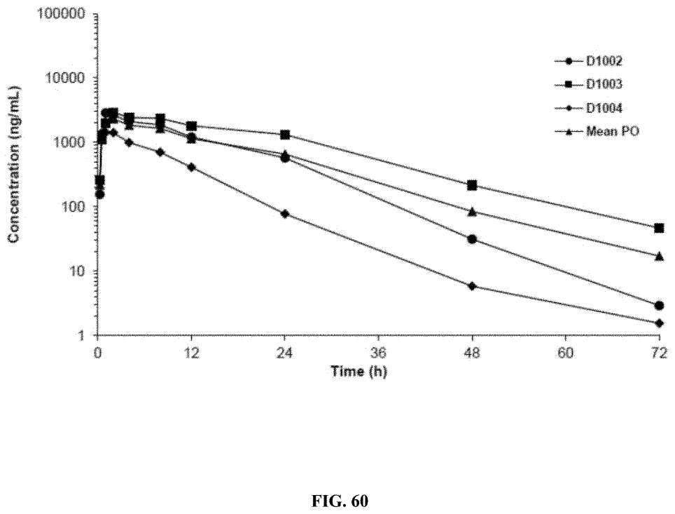

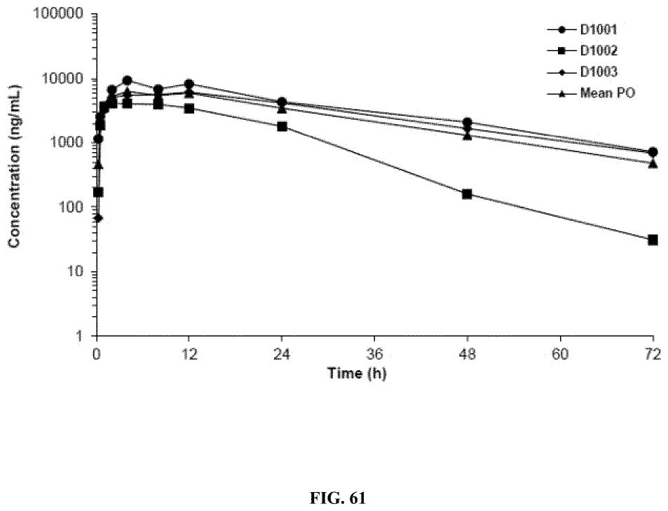

PK of Compound 2: FIG. 58 shows mean plasma concentration profiles of Compound 2 in male beagle dogs following single intravenous bolus and oral administrations of Compound 2 at 2.5, 25, and 100 mg/kg in phases 1, 2, and 3. TABLE 85 and FIG. 59 show individual and mean plasma concentrations of Compound 2 following IV bolus administration of 2.5 mg/kg Compound in male beagle dogs. TABLE 867 and FIG. 60 show individual and mean plasma concentrations of Compound 2 following PO administration of 25 mg/kg Compound in male beagle dogs. TABLE 87 and FIG. 61 show individual and mean plasma concentrations of Compound 2 following PO administration of 100 mg/kg Compound in male beagle dogs. The individual and mean plasma PK profiles are shown in TABLE 88, TABLE 89, and TABLE 90. The clinical chemistry and hematology test results are shown in TABLE 91 and TABLE 92.

TABLE 85

| IV Time | | | | | | CV |
|---|---|---|---|---|---|---|
| (h) | D1001¹ | D1002 | D1003 | Mean IV | SD | (%) |
| 0.0830 | 467 | 556 | 418 | 480 | 70.0 | 14.6 |
| 0.250 | 374 | 469 | 361 | 401 | 59.0 | 14.7 |
| 0.500 | 313 | 377 | 318 | 336 | 35.6 | 10.6 |
| 1.00 | 233 | 301 | 280 | 271 | 34.8 | 12.8 |
| 2.00 | 174 | 206 | 238 | 206 | 32.0 | 15.5 |
| 4.00 | 124 | 125 | 169 | 139 | 25.7 | 18.4 |
| 8.00 | 80.0 | 70.9 | 85.1 | 78.7 | 7.19 | 9.14 |
| 12.0 | 57.5 | 53.8 | 82.4 | 64.6 | 15.6 | 24.1 |
| 24.0 | 26.2 | 22.4 | 25.5 | 24.7 | 2.02 | 8.19 |
| 72.0 | BQL² | BQL | 3.13 | ND³ | ND | ND |

¹“D#” means animal number.
²“BQL” means below the quantifiable limit.
³“ND” means not determined due to less than 2 quantifiable values.

TABLE 86

| PO Time | | | | | | CV |
|---|---|---|---|---|---|---|
| (h) | D1002¹ | D1003 | D1004 | Mean PO | SD | (%) |
| 0.250 | 156 | 264 | 236 | 219 | 56.0 | 25.6 |
| 0.500 | 1350 | 1120 | 1380 | 1283 | 142 | 11.1 |
| 1.00 | 2880 | 1980 | 1430 | 2097 | 732 | 34.9 |
| 2.00 | 2660 | 2930 | 1420 | 2337 | 805 | 34.5 |
| 4.00 | 2110 | 2440 | 996 | 1849 | 757 | 40.9 |
| 8.00 | 1870 | 2370 | 703 | 1648 | 855 | 51.9 |
| 12.0 | 1210 | 1810 | 413 | 1144 | 701 | 61.2 |
| 24.0 | 574 | 1320 | 77.3 | 657 | 626 | 95.2 |
| 48.0 | 31.4 | 217 | 5.83 | 84.7 | 115 | 136 |
| 72.0 | 2.91 | 46.9 | 1.55 | 17.1 | 25.8 | 151 |

¹“D#” means animal number.

TABLE 87

| PO Time | | | | | | CV |
|---|---|---|---|---|---|---|
| (h) | D1001¹ | D1002 | D1003 | Mean PO | SD | (%) |
| 0.250 | 1150 | 173 | 68.5 | 464 | 597 | 129 |
| 0.500 | 2570 | 1870 | 1870 | 2103 | 404 | 19.2 |
| 1.00 | 3740 | 3640 | 3220 | 3533 | 276 | 7.81 |
| 2.00 | 6700 | 4140 | 5120 | 5320 | 1292 | 24.3 |
| 4.00 | 9380 | 4060 | 5520 | 6320 | 2749 | 43.5 |
| 8.00 | 6860 | 3980 | 5650 | 5497 | 1446 | 26.3 |
| 12.0 | 8280 | 3490 | 6150 | 5973 | 2400 | 40.2 |
| 24.0 | 4370 | 1810 | 4200 | 3460 | 1431 | 41.4 |
| 48.0 | 2100 | 160 | 1680 | 1313 | 1021 | 77.7 |
| 72.0 | 721 | 31.4 | 685 | 479 | 388 | 81.0 |

¹“D#” means animal number.

TABLE 88

| Parameter | D1001¹ | D1002 | D1003 | Mean IV | SD | CV (%) |
|---|---|---|---|---|---|---|
| Rsq_adj | 0.995 | 1.00 | 0.966 | —² | — | — |
| No. points used for T_{1/2} | 3.00 | 3.00 | 4.00 | ND³ | — | — |
| C₀ (ng/mL) | 521 | 605 | 450 | 525 | 77.8 | 14.8 |
| T_{1/2} (h) | 10.1 | 9.59 | 13.3 | 11.0 | 2.03 | 18.5 |
| Vdₛₛ (L/kg) | 13.1 | 11.4 | 12.4 | 12.3 | 0.848 | 6.90 |
| C_{L} (mL/min/kg) | 17.6 | 17.7 | 14.0 | 16.5 | 2.12 | 12.9 |
| Tₗₐₛₜ (h) | 24.0 | 24.0 | 72.0 | ND | — | — |
| AUC₀₋ₗₐₛₜ (ng · h/mL) | 1981 | 2042 | 2915 | 2313 | 522 | 22.6 |
| AUC_{0-inf} (ng · h/mL) | 2362 | 2352 | 2975 | 2563 | 357 | 13.9 |
| MRT₀₋ₗₐₛₜ (h) | 7.36 | 6.63 | 13.1 | 9.04 | 3.56 | 39.4 |
| MRT_{0-inf} (h) | 12.4 | 10.7 | 14.7 | 12.6 | 1.99 | 15.8 |
| AUC_{Extra} (%) | 16.1 | 13.2 | 2.02 | 10.4 | 7.44 | 71.2 |
| AUMC_{Extra} (%) | 50.2 | 46.4 | 12.5 | 36.4 | 20.7 | 57.0 |

¹“D#” means animal number.
²“—” means not applicable.
³“ND” means not determined.

TABLE 89

| Parameter | D1002¹ | D1003 | D1004 | Mean PO | SD | CV (%) |
|---|---|---|---|---|---|---|
| Rsq_adj | 0.993 | 0.996 | 0.975 | —² | — | — |
| No. points used for T_{1/2} | 3.00 | 3.00 | 7.00 | ND³ | — | — |
| Cₘₐₓ (ng/mL) | 2880 | 2930 | 1430 | 2413 | 852 | 35.3 |
| Tₘₐₓ (h) | 1.00 | 2.00 | 1.00 | 1.33 | 0.577 | 43.3 |
| T_{1/2} (h) | 6.30 | 9.97 | 6.85 | 7.71 | 1.98 | 25.7 |
| Tₗₐₛₜ (h) | 72.0 | 72.0 | 72.0 | 72.0 | — | — |
| AUC₀₋ₗₐₛₜ (ng · h/mL) | 37800 | 62673 | 13440 | 37971 | 24617 | 64.8 |
| AUC_{0-inf} (ng · h/mL) | 37827 | 63347 | 13455 | 38210 | 24948 | 65.3 |
| MRT₀₋ₗₐₛₜ (h) | 12.1 | 17.9 | 8.61 | 12.9 | 4.67 | 36.4 |
| MRT_{0-inf} (h) | 12.1 | 18.6 | 8.69 | 13.1 | 5.03 | 38.2 |
| AUC_{Extra} (%) | 0.0699 | 1.06 | 0.114 | 0.416 | 0.562 | 135 |
| AUMC_{Extra} (%) | 0.466 | 4.95 | 1.07 | 2.16 | 2.43 | 112 |
| Bioavailability (%)⁴ | 161 | 213 | 52.5 | 142 | 81.8 | 57.6 |

¹“D#” means animal number.
²“—” means not applicable.
³“ND” means not determined.
⁴Bioavailability (%) was calculated using AUC_{0-inf} (% AUC_{Extra} < 20%) or AUC0-last (% AUC_{Extra} > 20%) with nominal dose.

TABLE 90

| Parameter | D1001¹ | D1002 | D1003 | Mean PO | SD | CV (%) |
|---|---|---|---|---|---|---|
| Rsq_adj | 0.988 | 0.987 | 1.000 | —² | — | — |
| No. points used for T_{1/2} | 4.00 | 5.00 | 3.00 | ND³ | — | — |
| Cₘₐₓ (ng/mL) | 9380 | 4140 | 6150 | 6557 | 2644 | 40.3 |
| Tₘₐₓ (h) | 4.00 | 2.00 | 12.0 | 6.00 | 5.29 | 88.2 |
| T_{1/2} (h) | 17.7 | 8.76 | 18.3 | 14.9 | 5.35 | 35.9 |
| Tₗₐₛₜ (h) | 72.0 | 72.0 | 72.0 | 72.0 | — | — |
| AUC₀₋ₗₐₛₜ (ng · h/mL) | 264614 | 93666 | 216255 | 191512 | 88119 | 46.0 |
| AUC_{0-inf} (ng · h/mL) | 282995 | 94063 | 234386 | 203815 | 98106 | 48.1 |
| MRT₀₋ₗₐₛₜ (h) | 23.4 | 15.3 | 24.5 | 21.1 | 5.03 | 23.9 |
| MRT_{0-inf} (h) | 28.2 | 15.6 | 30.2 | 24.7 | 7.94 | 32.2 |
| AUC_{Extra} (%) | 6.50 | 0.422 | 7.74 | 4.88 | 3.91 | 80.1 |
| AUMC_{Extra} (%) | 22.4 | 2.29 | 25.2 | 16.6 | 12.5 | 75.1 |
| Bioavailability (%)⁴ | 300 | 100 | 197 | 199 | 99.8 | 50.2 |

¹“D#” means animal number.
²“—” means not applicable.
³“ND” means not determined.
⁴Bioavailability (%) was calculated using AUC_{0-inf} (% AUC_{Extra} < 20%) or AUC0-last (% AUC_{Extra} > 20%) with nominal dose.

TABLE 91

| | | | | Repeat | | | | | TP | | | Na | | sGLU | UREA | CRE |
|---|---|---|---|---|---|---|---|---|---|---|---|---|---|---|---|---|
| Time | Phase | Dose level | | Numbe | Sex/QC | Sample | ALT | AST | g/L | ALB | ALP | mmol/ | GGT | mmol/ | mmol/ | μmol/ |
| pre- | P1 | 2.5 | mg/kg | D1001 | Male | serum | 33 | 27 | 56.5 | 27.2 | 51 | 145 | 5 | 4.52 | 3.85 | 49 |
| dose | | | | D1002 | Male | serum | 25 | 28 | 51.7 | 25.6 | 130 | 147 | 2 | 5.56 | 4.27 | 44 |
| | | | | D1003 | Male | serum | 401 | 44 | 59.7 | 25.6 | 659 | 148 | 6 | 5.23 | 8.2 | 67 |
| 24 h | P1 | 2.5 | mg/kg | D1001 | Male | serum | 33 | 32 | 60.1 | 29 | 54 | 145 | 5 | 4.45 | 3.9 | 50 |
| | | | | D1002 | Male | serum | 26 | 30 | 53.6 | 27 | 128 | 148 | 3 | 5.75 | 4.88 | 44 |
| | | | | D1003 | Male | serum | 315 | 35 | 58 | 25.6 | 610 | 149 | 6 | 5.6 | 9.06 | 74 |
| pre- | P2 | 25 | mg/kg | D1001 | Male | serum | 31 | 33 | 60.5 | 29 | 53 | 146 | 6 | 4.49 | 3.74 | 52 |
| close | | | | D1002 | Male | serum | 25 | 31 | 53 | 27 | 127 | 148 | 4 | 5.54 | 3.96 | 45 |
| | | | | D1003 | Male | serum | 185 | 28 | 56.5 | 25.4 | 531 | 149 | 7 | 4.86 | 5.41 | 67 |
| | | | | D1004 | Male | Serum | 25 | 34 | 58.9 | 28.5 | 124 | 148 | 3 | 6.34 | 4.49 | 47 |
| 24 h | P2 | 25 | mg/kg | D1001 | Male | Serum | 28 | 28 | 60.2 | 29.2 | 51 | 145 | 6 | 5.58 | 4.67 | 46 |
| | | | | D1002 | Male | Serum | 25 | 25 | 53 | 26.8 | 126 | 147 | 3 | 5.99 | 3.13 | 39 |
| | | | | D1003 | Male | Serum | 160 | 29 | 60.4 | 27.8 | 518 | 151 | 7 | 5.18 | 6.83 | 68 |
| | | | | D1004 | Male | Serum | 35 | 34 | 59.4 | 29.3 | 126 | 147 | 3 | 5.85 | 4.05 | 43 |
| pre- | P3 | 100 | mg/kg | D1001 | Male | Serum | 26 | 32 | 60.2 | 28.7 | 52 | 148 | 5 | 4.82 | 5.26 | 49 |
| dose | | | | D1002 | Male | Serum | 26 | 32 | 54.8 | 27.7 | 133 | 148 | 3 | 5.81 | 3.81 | 45 |
| | | | | D1003 | Male | Serum | 103 | 27 | 54.8 | 25.8 | 408 | 147 | 6 | 5.28 | 5.84 | 60 |
| 24 h | P3 | 100 | mg/kg | D1001 | Male | Serum | 32 | 41 | 62.1 | 29.9 | 53 | 147 | 5 | 4.7 | 4 | 53 |
| | | | | D1002 | Male | Serum | 27 | 33 | 52.9 | 27.3 | 125 | 149 | 4 | 5.87 | 3.2 | 44 |
| | | | | D1003 | Male | Serum | 82 | 26 | 55.8 | 26.2 | 359 | 147 | 5 | 5.88 | 4.72 | 61 |

| | | | | | | | Ca | P | | TG | TBIL | K | Cl | | |
|---|---|---|---|---|---|---|---|---|---|---|---|---|---|---|---|
| Time | Phase | Dose level | | Repeat | Sex/QC | Sample | mm | mmol/ | TCHO | mmol/ | μmol/ | mmol/ | mmol/ | GLB | A/G |
| pre- | P1 | 2.5 | mg/kg | D1001 | Male | serum | 2.45 | 1.57 | 3.5 | 0.53 | 1.18 | 4.5 | 111 | 29.3 | 0.93 |
| dose | | | | D1002 | Male | serum | 2.48 | 2.27 | 5.43 | 0.59 | 1.13 | 4.7 | 112 | 26.1 | 0.98 |
| | | | | D1003 | Male | serum | 2.7 | 1.95 | 4.59 | 0.54 | 2.65 | 4.1 | 111 | 34.1 | 0.75 |
| 24 h | P1 | 2.5 | mg/kg | D1001 | Male | serum | 2.47 | 1.48 | 3.69 | 0.57 | 1.42 | 4.4 | 111 | 31.1 | 0.93 |
| | | | | D1002 | Male | serum | 2.56 | 2.64 | 5.47 | 0.51 | 1.22 | 4.6 | 111 | 26.6 | 1.02 |
| | | | | D1003 | Male | serum | 2.74 | 1.88 | 4.28 | 0.44 | 2.28 | 4.4 | 112 | 32.4 | 0.79 |
| pre- | P2 | 25 | mg/kg | D1001 | Male | serum | 2.49 | 1.58 | 3.66 | 0.57 | 1.28 | 4.6 | 112 | 31.5 | 0.92 |
| dose | | | | D1002 | Male | serum | 2.59 | 2.17 | 5.6 | 0.55 | 1.01 | 4.3 | 114 | 26 | 1.04 |
| | | | | D1003 | Male | serum | 2.69 | 2.14 | 3.91 | 0.42 | 1.81 | 4.4 | 113 | 31.1 | 0.82 |
| | | | | D1004 | Male | Seru | 2.78 | 2.26 | 4.93 | 0.63 | 1.28 | 4.5 | 110 | 30.4 | 0.94 |
| 24 h | P2 | 25 | mg/kg | D1001 | Male | Seru | 2.42 | 1.65 | 3.6 | 0.74 | 0.95 | 4.5 | 112 | 31 | 0.94 |
| | | | | D1002 | Male | Seru | 2.55 | 2.66 | 5.76 | 0.75 | 1.36 | 5 | 115 | 26.2 | 1.02 |
| | | | | D1003 | Male | Seru | 2.68 | 2.24 | 4.55 | 0.48 | 3.01 | 4.1 | 109 | 32.6 | 0.85 |
| | | | | D1004 | Male | Seru | 2.64 | 2.15 | 5.09 | 0.88 | 1.41 | 4.7 | 112 | 30.1 | 0.97 |
| pre- | P3 | 100 | mg/kg | D1001 | Male | Seru | 2.47 | 1.87 | 3.58 | 0.72 | 0.99 | 4.8 | 113 | 31.5 | 0.91 |
| dose | | | | D1002 | Male | Seru | 2.56 | 2.12 | 5.74 | 0.56 | 1.2 | 4.6 | 110 | 27.1 | 1.02 |
| | | | | D1003 | Male | Seru | 2.65 | 2.18 | 4.18 | 0.57 | 1.98 | 4.4 | 111 | 29 | 0.89 |
| 24 h | P3 | 100 | mg/kg | D1001 | Male | Seru | 2.5 | 1.59 | 3.93 | 0.63 | 5.64 | 4.6 | 113 | 32.2 | 0.93 |
| | | | | D1002 | Male | Seru | 2.48 | 2.17 | 6.05 | 0.64 | 2.42 | 4.7 | 116 | 25.6 | 1.07 |
| | | | | D1003 | Male | Seru | 2.61 | 2.16 | 4.78 | 0.6 | 4.74 | 4.4 | 112 | 29.6 | 0.89 |

TABLE 92

| | | | | Re- | Sex/ | Sam- | | RBC\hat{ }6/ | | | | | | | PLT\hat{ }3/ | | % NEUT |
|---|---|---|---|---|---|---|---|---|---|---|---|---|---|---|---|---|---|
| Time | Phase | Dose | | peat | QC | ple | WBC | μ | HGB | HCT | MCV | MCH | MCHC | RDW | μ | MPV | % |
| pre- | PI | 2.5 | mg/ [symbol] | D100 | Male | Whol | 9.93 | 5.69 | 13.1 | 39.9 | 70.2 | 23 | 32.7 | 13.4 | 306 | 11.6 | 50.8 |
| dose | | | | D100 | Male | Whol | 12.9 | 5.28 | 11.8 | 37.2 | 70.5 | 22.4 | 31.8 | 13.6 | 296 | 13.1 | 50.5 |
| | | | | D100 | Male | Whol | 13.5 | 5.87 | 13.1 | 40.5 | 69 | 22.3 | 32.4 | 13.9 | 368 | 11.4 | 61.5 |
| 24 h | PI | 2.5 | mg/ [symbol] | D100 | Male | Whol | 8.83 | 5.7 | 13.3 | 40.2 | 70.5 | 23.2 | 33 | 13.4 | 285 | 12.9 | 53.6 |
| | | | | D100 | Male | Whol | 10.4 | 5.42 | 12.2 | 38.3 | 70.5 | 22.6 | 32 | 13.9 | 317 | 15.5 | 46.3 |
| | | | | D100 | Male | Whol | 8.15 | 5.53 | 12.4 | 38.4 | 69.5 | 22.4 | 32.2 | 14.1 | 321 | 12.6 | 56.5 |
| pre- | P2 | 25 | mg/kg | D100 | Male | Whol | 10.1 | 5.94 | 13.8 | 41.6 | 69.9 | 23.2 | 33.1 | 13.8 | 291 | 11.9 | 52.6 |
| dose | | | | D100 | Male | Whol | 11.4 | 5.42 | 12.1 | 38.4 | 70.8 | 22.4 | 31.6 | 13.7 | 286 | 12.7 | 51.3 |
| | | | | D100 | Male | Whol | 11.8 | 5.68 | 12.5 | 39.1 | 69 | 22.1 | 32 | 14 | 296 | 11.6 | 62.9 |
| | | | | D100 | Male | Whol | 8.33 | 5.93 | 13.1 | 40.8 | 68.9 | 22.2 | 32.2 | 15 | 346 | 11.6 | 54.5 |
| 24 h | P2 | 25 | mg/kg | D100 | Male | Whol | 10.6 | 6.14 | 14.2 | 43.4 | 70.6 | 23.1 | 32.7 | 13.5 | 284 | 12.2 | 53.4 |
| | | | | D100 | Male | Whol | 15.3 | 5.49 | 12.8 | 39.4 | 71.6 | 23.2 | 32.4 | 13.6 | 285 | 12.1 | 53.9 |
| | | | | D100 | Male | Whol | 12.3 | 6.41 | 14 | 43.5 | 67.8 | 21.8 | 32.2 | 14 | 301 | 10.4 | 64.9 |
| | | | | D100 | Male | Whol | 8.92 | 6.05 | 13.4 | 41.5 | 68.6 | 22.2 | 32.4 | 15.3 | 346 | 10.9 | 55.6 |
| pre- | P3 | 100 | mg/ [symbol] | D100 | Male | Whol | 9.94 | 6.06 | 14 | 43 | 71 | 23 | 32.4 | 13.4 | 353 | 12.6 | 48.3 |
| dose | | | | D100 | Male | Whol | 11.3 | 5.53 | 12.5 | 38.9 | 70.4 | 22.6 | 32.1 | 13.9 | 308 | 14 | 49.8 |
| | | | | D100 | Male | Whol | 9.3 | 5.8 | 12.8 | 40.4 | 69.7 | 22.2 | 31.8 | 14.1 | 252 | 11.6 | 55.7 |
| 24 h | P3 | 100 | mg/ [symbol] | D100 | Male | Whol | 8.65 | 6.05 | 14 | 42.2 | 69.7 | 23.1 | 33.2 | 13.5 | 338 | 11.6 | 59 |
| | | | | D100 | Male | Whol | 13.4 | 5.05 | 11.6 | 35.5 | 70.3 | 23 | 32.7 | 13.7 | 311 | 13.9 | 54 |
| | | | | D100 | Male | Whol | 8.29 | 5.64 | 12.5 | 38.4 | 68.1 | 22.2 | 32.6 | 14.1 | 292 | 11.5 | 58 |

| | | | | Re- | Sex/ | Sam- | % | % | % | % | | | | | | % | |
|---|---|---|---|---|---|---|---|---|---|---|---|---|---|---|---|---|---|
| Tim | Phase | Dose | | peat | QC | ple | LYMP | MONO | EOS | BASO | #NEUT | #LYMP | #MONO | #EOS | #BASO | RET | #RET |
| pre- | P1 | 2.5 | mg/ [symbol] | D10 | Male | Who | 34 | 8.9 | 5.5 | 0.4 | 5.04 | 3.37 | 0.88 | 0.55 | 0.04 | 0.83 | 46.9 |
| dose | | | | D10 | Male | Who | 37.1 | 9.1 | 2.8 | 0.2 | 6.53 | 4.8 | 1.18 | 0.36 | 0.02 | 1.98 | 104.6 |
| | | | | D10 | Male | Who | 27.9 | 7.7 | 1.3 | 0.7 | 8.31 | 3.77 | 1.04 | 0.18 | 0.09 | 0.84 | 49.2 |
| 24 h | P1 | 2.5 | mg/ [symbol] | D10 | Male | Who | 32 | 9.2 | 4.5 | 0.4 | 4.73 | 2.82 | 0.81 | 0.39 | 0.03 | 0.76 | 43.1 |
| | | | | D10 | Male | Who | 42.1 | 7.8 | 3.3 | 0.2 | 4.83 | 4.38 | 0.81 | 0.35 | 0.02 | 1.92 | 104.1 |
| | | | | D10 | Male | Who | 34.2 | 4.3 | 3.1 | 0.8 | 4.6 | 2.79 | 0.35 | 0.25 | 0.07 | 0.63 | 35 |
| pre- | P2 | 25 | mg/kg | D10 | Male | Who | 32.6 | 8.4 | 4.8 | 0.8 | 5.35 | 3.32 | 0.85 | 0.49 | 0.08 | 0.74 | 43.7 |
| dose | | | | D10 | Male | Who | 36 | 7.8 | 4.4 | 0.1 | 5.85 | 4.11 | 0.89 | 0.51 | 0.02 | 1.9 | 103.2 |
| | | | | D10 | Male | Who | 24.9 | 8.8 | 2 | 0.5 | 7.43 | 2.94 | 1.04 | 0.24 | 0.06 | 0.98 | 55.6 |
| | | | | D10 | Male | Who | 34.2 | 7.4 | 2.9 | 0.5 | 4.54 | 2.85 | 0.62 | 0.24 | 0.04 | 0.95 | 56.6 |
| 24 h | P2 | 25 | mg/kg | D10 | Male | Who | 31.8 | 9.6 | 3.9 | 0.5 | 5.68 | 3.38 | 1.03 | 0.41 | 0.05 | 0.88 | 54 |
| | | | | D10 | Male | Who | 34.1 | 8.7 | 2.7 | 0.1 | 8.28 | 5.23 | 1.33 | 0.42 | 0.02 | 2.18 | 119.9 |
| | | | | D10 | Male | Who | 24.2 | 7.7 | 1.7 | 0.8 | 7.99 | 2.98 | 0.95 | 0.21 | 0.1 | 0.96 | 61.4 |
| | | | | D10 | Male | Who | 34.2 | 6.6 | 2.7 | 0.2 | 4.96 | 3.05 | 0.59 | 0.24 | 0.02 | 0.98 | 59.4 |
| pre- | P3 | 100 | mg/ [symbol] | D10 | Male | Who | 34.5 | 11.5 | 4.6 | 0.4 | 4.8 | 3.42 | 1.15 | 0.45 | 0.04 | 0.92 | 55.5 |
| dose | | | | D10 | Male | Who | 39.4 | 7.6 | 2.6 | 0.3 | 5.63 | 4.46 | 0.86 | 0.29 | 0.03 | 2.62 | 144.7 |
| | | | | D10 | Male | Who | 32.5 | 7.6 | 2.6 | 0.8 | 5.18 | 3.02 | 0.71 | 0.24 | 0.07 | 1.39 | 80.4 |
| 24 h | P3 | 100 | mg./ [symbol] | D10 | Male | Who | 27 | 8.6 | 4.1 | 0.5 | 5.1 | 2.33 | 0.74 | 0.36 | 0.04 | 0.79 | 47.8 |
| | | | | D10 | Male | Who | 35.6 | 7.3 | 2.5 | 0.2 | 7.28 | 4.79 | 0.98 | 0.34 | 0.03 | 2.18 | 110.2 |
| | | | | D10 | Male | Who | 32.5 | 6 | 1.5 | 0.8 | 4.81 | 2.7 | 0.5 | 0.13 | 0.07 | 1.02 | 57.6 |

[symbol]

indicates data missing or illegible when filed

After IV bolus administration at 2.5 mg/kg, concentrations of Compound 2 declined with a mean half-life at 11.0±2.03 h and a plasma clearance (CL) of 16.5±2.12 mL/min/kg. The volume of distribution (Vdₛₛ) was 12.3±0.848 L/kg and the area under the plasma concentration-time curve from time zero to the last quantifiable concentration (AUC₀₋ₗₐₛₜ) value was 2313±522 ng·h/mL.

After oral administration at 25 mg/kg or 100 mg/kg, Compound 2 was absorbed with a mean Cₘₐₓ value of 2413±852 ng/mL at a Tₘₐₓ of 1.33±0.577 for 25 mg/kg and a mean Cₘₐₓ value of 6557±2644 ng/mL at a Tₘₐₓ of 6.00±5.29 h for 100 mg/kg. The AUC₀₋ₗₐₛₜ for the 25 mg/kg and 100 mg/kg dose show a dose responsive increase of 37971 ng/mL and 191512 ng/mL, respectively. The mean percent oral bioavailability was greater than 100% at an oral dose of 25 mg/kg, which is likely due to the significant difference in dose levels between IV and PO administrations. TABLE 93 shows plasma PK data of Compound 2 following single IV bolus and oral administrations to non-naïve male beagle dogs.

TABLE 93

| | Phase | | | | | |
|---|---|---|---|---|---|---|
| | 1 | | 2 | | 3 | |
| | Dose Route | | | | | |
| | IV bolus | | PO | | | |
| | Dose Level (mg/kg) | | | | | |
| | 2.5 | | 25 | | 100 | |
| Parameter | Mean | SD | Mean | SD | Mean | SD |
| C₀ or Cₘₐₓ (ng/mL) | 525 | 77.8 | 2413 | 852 | 6557 | 2644 |
| Tₘₐₓ (h) | —¹ | — | 1.33 | 0.577 | 6.00 | 5.29 |
| T_{1/2} (h) | 11.0 | 2.03 | 7.71 | 1.98 | 14.9 | 5.35 |
| Vdₛₛ (L/kg) | 12.3 | 0.848 | — | — | — | — |
| C_{L} (mL/min/kg) | 16.5 | 2.12 | — | — | — | — |
| AUC₀₋ₗₐₛₜ (ng · h/mL) | 2313 | 522 | 37971 | 24617 | 191512 | 88119 |
| AUC_{0-inf} (ng · h/mL) | 2563 | 357 | 38210 | 24948 | 203815 | 98106 |
| Bioavailability (%)² | — | — | 142 | 81.8 | 199 | 99.8 |

¹“—” means not calculated.
²Bioavailability (%) was calculated using AUC_{0-inf} (% AUC_{Extra} < 20%) or AUC₀₋ₗₐₛₜ (% AUC_{Extra} > 20%) with nominal dose.

### Example 18: Plasma PK Study of Compound 2 Following Single IV Bolus and Oral Administrations to Non-Naïve Male Cynomolgus Monkeys

The PK properties of Compound 2 following single IV bolus or PO administration were investigated in male cynomolgus monkeys. In Phase 1, animals were administered with Compound 2 by single IV bolus administration at 2.5 mg/kg. In Phases 2 and 3, animals were administered with Compound 2 by single PO administration at 25 mg/kg and 100 mg/kg, respectively. Blood samples were collected at 0.083 hours (IV only), 0.25 h, 0.5 h, 1 h, 2 h, 4 h, 8 h, 12 h, 24 h, 48 h (PO only), and 72 h post-dose for Phases 1, 2, and 3. Clinical chemistry and hematology samples were collected at pre-dose (0 h) and 24 h post-dose. Concentrations of Compound 2 in plasma samples were determined by LC/MS/MS. Dosing and sampling regimens are described in TABLE 94 and TABLE 95.

TABLE 94

| | | | Dose | Target | | |
|---|---|---|---|---|---|---|
| | N | Dose | Volume | Dose Conc. | | Dosing |
| Group¹ | (gender) | (mg/kg) | (mL/kg) | (mg/mL) | Vehicle | Route |
| 1 | 3 Male | 2.5 | 2.5 | 1 | 2% DMA, 20% PEG400, and | IV |
| | | | | | 23% HPβCD w/v in H₂O | bolus |
| 2 | 3 Male | 25 | 5 | 5 | 2% Hydroxypropyl cellulose, | PO² |
| | | | | | 0.5% Polysorbate 80 v/v in H₂O | |
| 3 | 3 Male | 100 | 5 | 20 | 2% Hydroxypropyl cellulose, | PO² |
| | | | | | 0.5% Polysorbate 80 v/v in H₂O | |

¹Cross-over design, 3 days washout between dosing.
²For oral dosing, the animals were fasted overnight, food was returned at 4 hours post-dose.

TABLE 95

| | Dose | Animal | Sampling time point (h) ¹ | | | | | | | | | | |
|---|---|---|---|---|---|---|---|---|---|---|---|---|---|
| Group | (mg/kg) | No. | 0.083 | 0.25 | 0.5 | 1 | 2 | 4 | 8 | 12 | 24 | 48 | 72 |
| 1 | 2.5 | C1001, | P² | P | P | P | P | P | P | P | P | —³ | P |
| | | C1002, | | | | | | | | | | | |
| | | C1003 | | | | | | | | | | | |
| 2 | 25 | C1001, | — | P | P | P | P | P | P | P | P | P | P |
| | | C1002, | | | | | | | | | | | |
| | | C1003 | | | | | | | | | | | |
| 3 | 100 | C1001, | — | P | P | P | P | P | P | P | P | P | P |
| | | C1002, | | | | | | | | | | | |
| | | C1003 | | | | | | | | | | | |

¹ Blood collection for clinical chemistry and hematology was sampled at pre-dose (0 h) and 24 hours post-dose.
²P = plasma.
³“—” means no sample collection.

Formulations: The IV vehicle for Phase I was 2% DMA, 20% PEG400, and 23% HPβCD w/v in water at 2.5 mg/kg. The PO vehicle for Phase 2 and 3 was 2% Hydroxypropyl cellulose, 0.5% Polysorbate 80 v/v in water at 5 mg/mL and 20 mg/mL. All dosing solutions were analyzed for Compound 2 concentration by a LC/UV method. Two aliquots were taken from the middle region of the IV dosing solution. Two aliquots were taken from the bottom, middle, and top regions of the PO dosing formulation. All formulation samples were stored at 2-8° C. until analysis by LC/UV. Acceptance criteria: The measured concentrations of test article in each dose formulation were within 80% to 120% of the nominal concentrations.

Animals: Three non-naïve Cynomolgus monkeys were supplied by Hainan Jingang Laboratory Animal Co., Ltd. (n=3, see Section 2.2). The animals were confirmed healthy before assignment to this study. A unique identification number was marked on the chest and cage card of each study animal. The animal room environment was controlled and monitored for temperature (18-26° C.) and relative humidity (40-70%) with 10 to 20 air changes/hour. The room was on a 12-hour light/dark cycle except when interruptions are necessitated by study activities. Any temperature excursion from the targeted mean range of 18-26° C., was documented as a deviation. Any relative humidity excursion from the targeted mean range of 40-70% for more than 3 hours was documented as a deviation. Fresh drinking water was available to all animals, ad libitum. Animals were fed twice daily. Stock monkeys were fed approximately 120 grams of Certified Monkey Diet daily. These amounts were adjusted as necessary based on food consumption of the group or an individual body weight change. In addition, animals received fruit daily as nutritional enrichment. For PO dose phase, animals were fed the afternoon (at 3:30 to 4:00 pm) prior to the day of oral dosing and the remaining food were removed at 7:00 pm. Food was withheld until 4-hour post-dose unless specified in this protocol. Animals were fed approximately 120 grams certified diet once on the day of dosing. Animals were weighed prior to dose administration on each day of dosing to calculate the actual dose volume. The body weights were in the range from 2.96 to 3.55 kg for males on the first dosing day. Cage-side animal observations for appearance and general health condition were performed before and after dosing as well as at each time point of sample collection.

Plasma sample for PK analysis: Approximately 0.5 mL blood was collected at each time point via peripheral vessel from each study animal. The actual time for each sample collection was recorded. Deviations on sampling time were less than 1 minute for the time points pre-dose through 1 hour post-dose, and less than 5% of the nominal time for time points after 1 hour post-dose. All blood samples were transferred into commercial collection tubes containing K₂EDTA (0.85-1.15 mg). Plasma samples were then prepared by spinning by centrifuge the blood samples at approximately 2 to 8° C., 3000× g for 10 minutes. All plasma samples were then quickly frozen over dry ice and kept at −60° C. or lower until LC/MS/MS analysis.

For serum samples for clinical chemistry analysis, whole blood samples (approximately 1.0 mL) without anticoagulant were collected and held at room temperature and up-right for at least 30 minutes and sent to clinical pathology lab for analysis. For blood samples for hematology analysis, whole blood (at least 1.0 mL) was collected from the experimental animals into tubes with K₂EDTA at room temperature. The concentrations of Compound 2 in plasma were determined by using a LC/MS/MS method

PK Data Analysis: The plasma concentration of Compound 2 in study animals was subjected to a non-compartmental PK analysis. The linear/log trapezoidal rule was applied in obtaining the PK parameters. Individual plasma concentration values that were below the LLOQ were excluded from the PK parameter calculation. All plasma concentrations and PK parameters were reported with three significant figures. The nominal dose levels and nominal sampling times were used in the calculation of all PK parameters. TABLE 96 shows PK data analysis following single IV Bolus and PO administrations to non-naïve male cynomolgus monkeys.

TABLE 96

| | | | | | Measured | Calculated | Mean | Nominal | |
|---|---|---|---|---|---|---|---|---|---|
| Phase | Dose | Sample | | Dilution | Conc. | Conc. | Conc. | Conc. | Accuracy ^{a} |
| No. | Route | No. | Determination | Factor | (μg/mL) | (mg/mL) | (mg/mL) | (mg/mL) | (%) |
| 1 | [Pre] | Middle 1 | 1.00 | 20.0 | 52.1 | 1.04 | 1.03 | 1.00 | 103 |
| | IV | | 2.00 | | 51.8 | 1.04 | | | |
| | bolus | Middle 2 | 1.00 | | 50.5 | 1.01 | | | |
| | | | 2.00 | | 51.6 | 1.03 | | | |
| 2 | [Pre] | Top | 1.00 | 50.0 | 109 | 5.45 | 5.32 | 5.00 | 106 |
| | PO | | 2.00 | | 109 | 5.45 | | | |
| | | Middle | 1.00 | | 106 | 5.32 | | | |
| | | | 2.00 | | 107 | 5.33 | | | |
| | | Bottom | 1.00 | | 104 | 5.21 | | | |
| | | | 2.00 | | 104 | 5.20 | | | |
| 3 | [Pre] | Top | 1.00 | 400 | 50.0 | 20.0 | 19.4 | 20.0 | 96.8 |
| | PO | | 2.00 | | 50.2 | 20.1 | | | |
| | | Middle | 1.00 | | 48.1 | 19.2 | | | |
| | | | 2.00 | | 47.9 | 19.2 | | | |
| | | Bottom | 1.00 | | 47.1 | 18.8 | | | |
| | | | 2.00 | | 47.2 | 18.9 | | | |

^{a} Accuracy (%) = Mean Concentration (mg/mL)/Nominal Concentration (mg/mL) × 100.

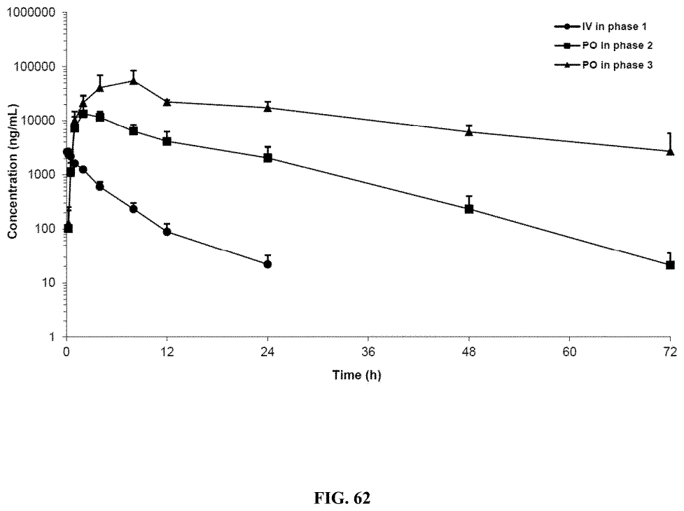

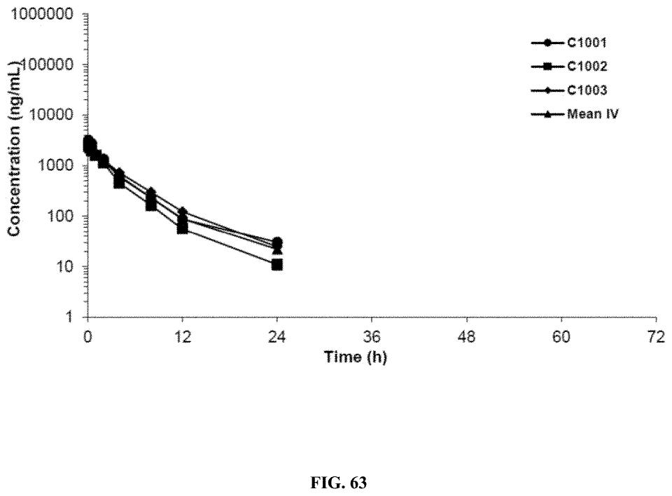

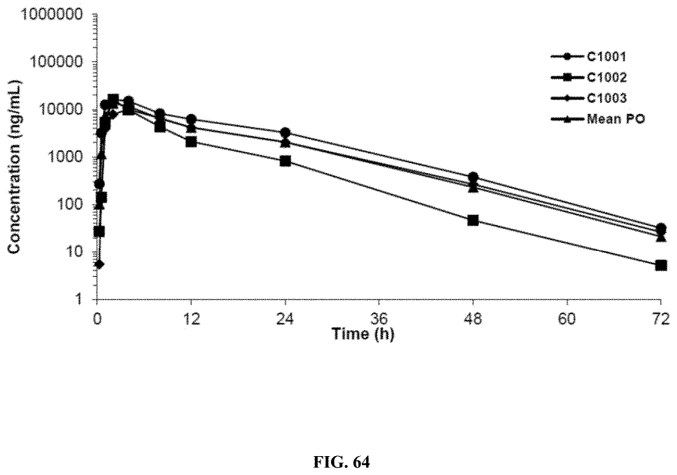

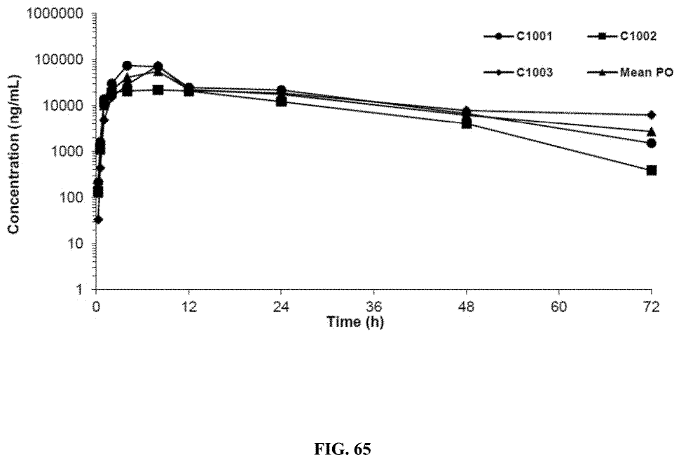

PK of Compound 2 in Cynomolgus Monkeys: TABLE 97 and FIG. 63 show individual and mean plasma concentrations of Compound 2 in male cynomolgus monkeys following IV bolus administration at 2.5 mg/kg. TABLE 98, TABLE 99, FIG. 64, and FIG. 65 show individual and mean plasma concentrations of Compound 2 in male cynomolgus monkeys following a single PO administration at 25 mg/kg and 100 mg/kg. The mean plasma PK parameters of Compound 2 both IV and PO are shown in TABLE 100 and FIG. 62. The individual and mean plasma PK profiles are shown in TABLE 101, TABLE 102, and TABLE 103. The clinical chemistry and hematology test results are shown m TABLE 104 and TABLE 105.

TABLE 97
Plasma concentration of Compound 2
Male cynomolgus monkeys; IV bolus administration 2.5 mg/kg

| IV Time | | | | | | CV |
|---|---|---|---|---|---|---|
| (h) | C1001¹ | C1002 | C1003 | Mean IV | SD | (%) |
| 0.0830 | 3180 | 2390 | 2420 | 2663 | 448 | 16.8 |
| 0.250 | 2650 | 2240 | 2230 | 2373 | 240 | 10.1 |
| 0.500 | 2720 | 1930 | 1950 | 2200 | 450 | 20.5 |
| 1.00 | 1640 | 1590 | 1650 | 1627 | 32.1 | 1.98 |
| 2.00 | 1340 | 1140 | 1280 | 1253 | 103 | 8.19 |
| 4.00 | 624 | 452 | 718 | 598 | 135 | 22.6 |
| 8.00 | 233 | 165 | 298 | 232 | 66.5 | 28.7 |
| 12.0 | 87.7 | 55.8 | 123 | 88.8 | 33.6 | 37.8 |
| 24.0 | 30.2 | 10.9 | 24.6 | 21.9 | 9.93 | 45.3 |
| 72.0 | BQL² | BQL | BQL | ND³ | ND | ND |

¹“C#” means animal number.
²“BQL” means below the quantifiable limit.
³“ND” means not determined due to less than 2 quantifiable values.

TABLE 98
Plasma concentration of Compound 2
Male cynomolgus monkeys; PO administration 25 mg/kg

| PO Time | | | | | | CV |
|---|---|---|---|---|---|---|
| (h) | C1001¹ | C1002 | C1003 | Mean PO | SD | (%) |
| 0.250 | 273 | 27.5 | 5.49 | 102 | 149 | 146 |
| 0.500 | 3210 | 144 | 127 | 1160 | 1775 | 153 |
| 1.00 | 12600 | 5330 | 3970 | 7300 | 4640 | 63.6 |
| 2.00 | 16300 | 16300 | 7900 | 13500 | 4850 | 35.9 |
| 4.00 | 15100 | 9810 | 10000 | 11637 | 3001 | 25.8 |
| 8.00 | 8200 | 4360 | 6620 | 6393 | 1930 | 30.2 |
| 12.0 | 6220 | 2120 | 4170 | 4170 | 2050 | 49.2 |
| 24.0 | 3260 | 829 | 2060 | 2050 | 1216 | 59.3 |
| 48.0 | 381 | 46.8 | 267 | 232 | 170 | 73.4 |
| 72.0 | 31.9 | 5.15 | 26.4 | 21.2 | 14.1 | 66.8 |

¹“C#” means animal number.

TABLE 99
Plasma concentration of Compound 2
Male cynomolgus monkeys; PO administration 100 mg/kg

| PO Time | | | | | | CV |
|---|---|---|---|---|---|---|
| (h) | C1001¹ | C1002 | C1003 | Mean PO | SD | (%) |
| 0.250 | 213 | 135 | 33.5 | 127 | 90.0 | 70.8 |
| 0.500 | 1610 | 1190 | 436 | 1079 | 595 | 55.1 |
| 1.00 | 13800 | 11800 | 4850 | 10150 | 4698 | 46.3 |
| 2.00 | 29900 | 19400 | 15100 | 21467 | 7613 | 35.5 |
| 4.00 | 73000 | 20700 | 28200 | 40633 | 28280 | 69.6 |
| 8.00 | 69500 | 21900 | 73200 | 54867 | 28610 | 52.1 |
| 12.0 | 24600 | 20800 | 21300 | 22233 | 2065 | 9.29 |
| 24.0 | 21700 | 12100 | 18600 | 17467 | 4899 | 28.0 |
| 48.0 | 6720 | 4030 | 7800 | 6183 | 1941 | 31.4 |
| 72.0 | 1510 | 385 | 6230 | 2708 | 3101 | 115 |

¹“C#” means animal number.

TABLE 100

| | Phase | | | | | |
|---|---|---|---|---|---|---|
| | 1 | | 2 | | 3 | |
| | Dose Route | | | | | |
| | IV bolus | | PO | | PO | |
| | Dose level (mg/kg) | | | | | |
| | 2.5 | | 25 | | 100 | |
| PK parameters | Mean | SD | Mean | SD | Mean | SD |
| C₀ or Cₘₐₓ (ng/mL) | 2823 | 571 | 14200 | 3637 | 56033 | 29561 |
| Tₘₐₓ (h) | —¹ | — | 2.67 | 1.15 | 6..67 | 2.31 |
| T_{1/2} (h) | 3.95 | 0.588 | 7.14 | 0.517 | 18.0 | 11.2 |
| V_{dss} (L/kg) | 1.32 | 0.0682 | — | — | — | — |
| C1 (mL/min/kg) | 5.17 | 0.866 | — | — | — | — |
| AUC₀₋ₗₐₛₜ (ng · h/mL) | 8078 | 1199 | 151182 | 58128 | 1007009 | 327373 |
| AUC_{0-inf} (ng · h/mL) | 8203 | 1255 | 151406 | 58256 | 1110757 | 411099 |
| ²Bioavailability (%) | — | — | 182 | 53.1 | 330 | 81.6 |

¹“—” means not calculated.
²Bioavailability (%) was calculated using AUC_{0-inf} (% AUC_{Extra<20%}) or AUC₀₋ₗₐₛₜ (% AUC_{Extra>20%}) with minimal dose.

TABLE 101
Plasma PK profiles
Male cynomolgus monkeys; IV bolus administration 2.5 mg/kg

| PK Parameters | C1001¹ | C1002 | C1003 | Mean IV | SD | CV (%) |
|---|---|---|---|---|---|---|
| Rsq_adj | 0.906 | 0.953 | 0.974 | —² | — | — |
| No. points used for T_{1/2} | 9.00 | 4.00 | 3.09 | ND³ | — | — |
| C₀ (ng/mL) | 3482 | 2468 | 2520 | 2823 | 571 | 20.2 |
| T_{1/2} (h) | 344 | 3.82 | 4.59 | 3.95 | 0.588 | 14.9 |
| V_{dss} (L/kg) | 124 | 1.35 | 1.37 | 1.32 | 0.0682 | 5.17 |
| C₁ (mL/min/kg) | 4.71 | 6.17 | 4.62 | 5.17 | 0.866 | 16.7 |
| Tₗₐₛₜ (h) | 24.0 | 24.0 | 24.0 | 24.0 | — | — |
| AUC₀₋ₗₐₛₜ (ng · h/mL) | 8689 | 6698 | 8849 | 8078 | 1199 | 14.8 |
| AUC_{0-inf} (ng · h/mL) | 8838 | 6758 | 9012 | 8203 | 1255 | 15.3 |
| MRT₀₋ₗₐₛₜ (h) | 3.97 | 3.42 | 4.45 | 3.95 | 0.516 | 13.1 |
| MRT_{0-inf} (h) | 439 | 3.65 | 4.93 | 4.32 | 0.639 | 14.8 |
| AUC_{Extra} (%) | 1.70 | 0888 | 1.81 | 1.46 | 0.502 | 3.43 |
| AUMC_{Extra} (%) | 11.2 | 7.18 | 11.2 | 9.87 | 2.33 | 23.6 |

¹“—” means animal number.
²“—” means not applicable.
³“ND” means not determined.

TABLE 102
Plasma PK Profiles
Male cynomolgus monkeys; PO administration 25 mg/kg

| PK Parameters | C1001¹ | C1002 | C1003 | Mean PO | SD | CV (%) |
|---|---|---|---|---|---|---|
| Rsq_adj | 0.997 | 0.995 | 0.997 | —² | — | — |
| No. points used for T_{1/2} | 3.00 | 5.00 | 3.00 | ND³ | — | — |
| Cₘₐₓ (ng/mL) | 16300 | 16300 | 10000 | 14200 | 3637 | 25.6 |
| Tₘₐₓ (h) | 2.00 | 2.00 | 4.00 | 2.67 | 1.15 | 43.3 |
| T_{1/2} (h) | 7.19 | 6.61 | 7.64 | 7.14 | 0.517 | 7.24 |
| Tₗₐₛₜ (h) | 72.0 | 72.0 | 72.0 | 71.0 | — | — |
| AUC₀₋ₗₐₛₜ (ng · h/mL) | 214665 | 100564 | 138318 | 151182 | 58128 | 38.4 |
| AUC_{0-inf} (ng · h/mL) | 214996 | 100613 | 138609 | 151406 | 58256 | 38.5 |
| MRT₀₋ₗₐₛₜ (h) | 13.2 | 8.74 | 13.8 | 11.9 | 2.76 | 23.2 |
| MRT_{0-inf} (h) | 13.3 | 8.78 | 13.9 | 12.0 | 2.81 | 23.4 |
| AUC_{Extra} (%) | 0.154 | 0.0488 | 0.210 | 0.138 | 0.0818 | 59.5 |
| AUMC_{Extra} (%) | 0.951 | 0.453 | 1.25 | 0.885 | 0.403 | 45.5 |
| Bioavailability (%)⁴ | 243 | 149 | I54 | 182 | 53.1 | 29.2 |

¹“C#” means animal number.
²“—” means not applicable.
³“ND” means not determined.
⁴Bioavailability (%) was calculated using AUC_{0-inf} (% AUC_{Extra<20%}) or AUC₀₋ₗₐₛₜ (% AUC_{Extra>20%}) with minimal dose

TABLE 103
Plasma PK Profiles
Male cynomolgus monkeys; PO administration 100 mg/kg

| PK Parameters | C1001¹ | C1002 | C1003 | Mean PO | SD | CV (%) |
|---|---|---|---|---|---|---|
| Rsq_adj | 0.990 | 0.940 | 0.908 | —² | — | — |
| No. points used for T_{1/2} | 3.00 | 400 | 4.00 | ND³ | — | — |
| Cₘₐₓ (ng/mL) | 73000 | 21900 | 73200 | 56033 | 29561 | 52.8 |
| Tₘₐₓ (h) | 4.00 | 8.00 | 8.00 | 6.67 | 2.31 | 34.6 |
| T_{1/2} (h) | 12.5 | 10.6 | 30.9 | 18.0 | 11.2 | 62.3 |
| Tₗₐₛₜ (h) | 72.0 | 72.0 | 72.0 | 723 | — | — |
| AUC₀₋ₗₐₛₜ (ng · h/mL) | 1254615 | 635836 | 1130576 | 1007009 | 327373 | 32.5 |
| AUC_{0-inf} (ng · h/mL) | 1281810 | 641749 | I4057I1 | I110757 | 411099 | 37.0 |
| MRT₀₋ₗₐₛₜ (h) | 19.0 | 20.3 | 24.5 | 213 | 2.84 | 13.4 |
| MRT_{0-inf} (h) | 20.5 | 20.9 | 42.7 | 28.0 | 12.7 | 45.2 |
| AUC_{Extra} (%) | 2.12 | 0.921 | 19.7 | 7.60 | 10.5 | 139 |
| AUMC_{Extra} (%) | 9.31 | 3.84 | 34.0 | 22.4 | 27.5 | 123 |
| Bioavailability (%)⁴ | 363 | 237 | 391 | 330 | 81.6 | 24.7 |

¹“C#” means animal number.
²“—” means not applicable.
³“ND” means not determined.
⁴Bioavailability (%) was calculated using AUC_{0-inf} (% AUC_{Extra<20%}) or AUC₀₋ₗₐₛₜ (% AUC_{Extra>20%}) with minimal dose

TABLE 104
Chemistry and hematology test results

| | | | | Repeat | Sex QC | Sample | ALT | AST | TP | ALB | ALB | Na | GGT | sGLU | UREA | CRE |
|---|---|---|---|---|---|---|---|---|---|---|---|---|---|---|---|---|
| Time | Phase | Dose level | | Number | Identifier | Type | U/L | U/L | g/L | g L | U/L | mmol/L | U/L | mmol/L | mmol/L | μmol/L |
| pre- | P1 | 2.5 | mg/kg | C1001 | Male | serum | 68 | 40 | 74.6 | 41.9 | . | 148 | 89 | 2.96 | 4.97 | 44 |
| dose | | | | C1002 | Male | serum | 58 | 58 | 79.9 | 41.1 | 509 | 147 | 58 | 3.39 | 5.66 | 55 |
| | | | | C1003 | Male | serum | 42 | 43 | 75.1 | 40 | 541 | 145 | 60 | 2.9 | 5.82 | 53 |
| 24 h | P1 | 2.5 | mg/kg | C1001 | Male | serum | 90 | 76 | 77 | 44.3 | 645 | 155 | 92 | 3.15 | 5.04 | 55 |
| | | | | C1002 | Male | serum | 70 | 85 | 77.1 | 40.2 | 454 | 150 | 55 | 2.45 | 6.21 | 60 |
| | | | | C1003 | Male | serum | 41 | 50 | 76.8 | 41.6 | 517 | 149 | 62 | 2.81 | 5.48 | 55 |
| pre- | P2 | 25 | mg/kg | C1001 | Male | serum | 73 | 43 | 76.1 | 44 | 597 | 149 | 91 | 2.7 | 5.02 | 49 |
| dose | | | | C1002 | Male | serum | 62 | 62 | 82.2 | 42 | 435 | 148 | 59 | 2.79 | 6.58 | 57 |
| | | | | C1003 | Male | serum | 41 | 50 | 78.9 | 42.8 | 506 | 148 | 67 | 2.93 | 5.46 | 52 |
| 24 h | P2 | 25 | mg/kg | C1001 | Male | Serum | 71 | 38 | 70.4 | 41.4 | 550 | 151 | 83 | 3.58 | 4.74 | 57 |
| | | | | C1002 | Male | Serum | 54 | 39 | 82.4 | 42.9 | 421 | 153 | 59 | 3.99 | 5.58 | 64 |
| | | | | C1003 | Male | Serum | 38 | 35 | 79 | 42.6 | 488 | 148 | 66 | 3.51 | 4.14 | 57 |
| pre- | P3 | 100 | mg/kg | C1001 | Male | Serum | 59 | 31 | 72.9 | 41.9 | 516 | 149 | 84 | 4.17 | 4.54 | 50 |
| dose | | | | C1002 | Male | Serum | 45 | 52 | 79 | 40.1 | 377 | 150 | 55 | 3.5 | 6.21 | 57 |
| | | | | C1003 | Male | Serum | 31 | 36 | 80.3 | 43.5 | 460 | 146 | 67 | 3.41 | 5.97 | 51 |
| 24 h | P3 | 100 | mg/kg | C1001 | Male | Serum | 68 | 45 | 74.1 | 42.9 | 514 | 148 | 85 | 3.11 | 4.4 | 60 |
| | | | | C1002 | Male | Serum | 47 | 51 | 76.6 | 40.4 | 370 | 151 | 55 | 3.44 | 5.13 | 72 |
| | | | | C1003 | Male | Serum | 32 | 36 | 76.5 | 41.4 | 431 | 147 | 63 | 3.05 | 4.55 | 66 |

| | | | | Repeat | Sex/QC | Sample | Ca | P | TCHO | TG | TBIL | K | Cl | GLB | |
|---|---|---|---|---|---|---|---|---|---|---|---|---|---|---|---|
| Time | Phase | Dose level | | Number | Identifier | Type | mmol/L | mmol/L | mmol/L | mmol/L | μmol/L | mmol/L | mmol/LL | g/ | A/G |
| pre- | P1 | 2.5 | mg/kg | C1001 | Male | serum | 2.37 | 2.43 | 3.41 | 0.59 | 2.82 | 5 | 105 | 32.7 | 1.28 |
| dose | | | | C1002 | Male | serum | 2.48 | 2.5 | 3.91 | 0.42 | 2.4 | 4.5 | 107 | 38.8 | 1.06 |
| | | | | C1003 | Male | serum | 2.43 | 1.79 | 3.19 | 0.76 | 1.61 | 4.4 | 107 | 35.1 | 1.14 |
| 24 h | P1 | 2.5 | mg/kg | C1001 | Male | serum | 2.36 | 2.34 | 3.24 | 0.4 | 4.06 | 5.2 | 110 | 32.7 | 1.35 |
| | | | | C1002 | Male | serum | 2.42 | 2.28 | 3.78 | 0.3 | 3.01 | 4.3 | 110 | 36.9 | 1.09 |
| | | | | C1003 | Male | serum | 2.51 | 1.78 | 3.28 | 0.4 | 2.04 | 4.4 | 109 | 35.2 | 1.18 |
| pre- | P2 | 25 | mg/kg | C1001 | Male | serum | 2.38 | 2.38 | 3.31 | 0.35 | 3.03 | 4.6 | 106 | 32.1 | 1.37 |
| dose | | | | C1002 | Male | serum | 2.55 | 2.69 | 4.52 | 0.41 | 2.37 | 5.5 | 108 | 40.2 | 1.04 |
| | | | | C1003 | Male | serum | 2.57 | 2.33 | 3.57 | 0.44 | 2.4 | 4.7 | 107 | 36.1 | 1.19 |
| 24 h | P2 | 25 | mg/kg | C1001 | Male | Serum | 2.35 | 2.23 | 3.01 | 0.3 | 7.66 | 4.9 | 110 | 29 | 1.43 |
| | | | | C1002 | Male | Serum | 2.55 | 2.55 | 4.6 | 0.26 | 6.86 | 5.1 | 110 | 39.5 | 1.09 |
| | | | | C1003 | Male | Serum | 2.56 | 2.02 | 3.59 | 0.29 | 7.45 | 5.1 | 108 | 36.4 | 1.17 |
| pre- | P3 | 100 | mg/kg | C1001 | Male | Serum | 2.38 | 2.04 | 3.24 | 0.34 | 2.55 | 4.7 | 105 | 31 | 1.35 |
| dose | | | | C1002 | Male | Serum | 2.52 | 2.42 | 4.37 | 0.4 | 2.6 | 5.1 | 107 | 38.9 | 1.03 |
| | | | | C1003 | Male | Serum | 2.56 | 1.93 | 3.55 | 0.74 | 3.46 | 5 | 106 | 36.8 | 1.18 |
| 24 h | P3 | 100 | mg/kg | C1001 | Male | Serum | 2.32 | 1.75 | 3.57 | 0.58 | 17.36 | 4 | 107 | 31.2 | 1.38 |
| | | | | C1002 | Male | Serum | 2.56 | 2.3 | 4.37 | 0.45 | 23.82 | 5.1 | 109 | 36.2 | 1.12 |
| | | | | C1003 | Male | Serum | 2.49 | 1.57 | 3.76 | 0.7 | 23.39 | 4.5 | 108 | 35.1 | 1.18 |

TABLE 105
Chemistry and hematology test results

| | | | | | Sex/QC | | | | | | | | | | | | % |
|---|---|---|---|---|---|---|---|---|---|---|---|---|---|---|---|---|---|
| | | | | Repeat | Iden- | Sample | WBC\hat{ }3/ | RBC\hat{ }6/ | HGB | HCT | MCV | MCH | MCHC | RDW | PLT\hat{ }3/ | MPV | NEUT |
| Time | Phase | Dose level | | No. | tifier | Type | μL | μL | g/dL | % | fL | pg | g/dL | % | μL | fL | % |
| pre- | P1 | 2.5 | mg/kg | C1001 | Male | Whole | 8.42 | 5.55 | 13.1 | 43.8 | 78.8 | 23.6 | 30 | 13.7 | 524 | 8.5 | 17.9 |
| dose | | | | | | Blood | | | | | | | | | | | |
| | | | | C1002 | Male | Whole | 7.72 | 4.7 | 11.9 | 38.5 | 81.9 | 25.2 | 30.8 | 13.6 | 576 | 9.1 | 10.2 |
| | | | | | | Blood | | | | | | | | | | | |
| | | | | C1003 | Male | Whole | 14.41 | 5.63 | 11.9 | 39.8 | 70.7 | 21.1 | 29.8 | 14.8 | 377 | 9.1 | 16.1 |
| | | | | | | Blood | | | | | | | | | | | |
| 24 h | P1 | 2.5 | mg/kg | C1001 | Male | Whole | 9.53 | 5.15 | 12.1 | 40.8 | 79.2 | 23.5 | 29.6 | 13.8 | 497 | 9.5 | 57.3 |
| | | | | | | Blood | | | | | | | | | | | |
| | | | | C1002 | Male | Whole | 6.34 | 4.3 | 10.6 | 34.9 | 81.1 | 24.7 | 30.5 | 13.7 | 578 | 9.4 | 17.1 |
| | | | | | | Blood | | | | | | | | | | | |
| | | | | C1003 | Male | Whole | 11.78 | 5.39 | 11.5 | 38.1 | 70.8 | 21.4 | 30.2 | 15 | 369 | 9.9 | 35.1 |
| | | | | | | Blood | | | | | | | | | | | |
| pre- | P2 | 25 | mg/kg | C1001 | Male | Whole | 7.15 | 5.34 | 12.6 | 41.6 | 77.9 | 23.6 | 30.2 | 13.9 | 472 | 8.7 | 25.7 |
| dose | | | | | | Blood | | | | | | | | | | | |
| | | | | C1002 | Male | Whole | 9.48 | 4.43 | 11.1 | 35.4 | 79.8 | 25.1 | 31.4 | 13.9 | 648 | 9 | 19.5 |
| | | | | | | Blood | | | | | | | | | | | |
| | | | | C1003 | Male | Whole | 14.69 | 5.69 | 12.1 | 39.6 | 69.6 | 21.2 | 30.4 | 14.9 | 405 | 9.2 | 28.4 |
| | | | | | | Blood | | | | | | | | | | | |
| 24 h | P2 | 25 | mg/kg | C1001 | Male | Whole | 5.89 | 5.02 | 11.8 | 39.3 | 78.3 | 23.6 | 30.1 | 14 | 451 | 8.8 | 23.9 |
| | | | | | | Blood | | | | | | | | | | | |
| | | | | C1002 | Male | Whole | 7.37 | 4.46 | 11 | 36.9 | 82.7 | 24.7 | 29.9 | 13.9 | 669 | 9.2 | 7.1 |
| | | | | | | Blood | | | | | | | | | | | |
| | | | | C1003 | Male | Whole | 10.21 | 5.72 | 12.1 | 40.2 | 70.2 | 21.1 | 30 | 15.2 | 384 | 8.8 | 21.3 |
| | | | | | | Blood | | | | | | | | | | | |
| pre- | P3 | 100 | mg/kg | C1001 | Male | Whole | 9.39 | 4.88 | 11.8 | 38.8 | 79.6 | 24.1 | 30.3 | 13.7 | 522 | 8.6 | 33.4 |
| dose | | | | | | Blood | | | | | | | | | | | |
| | | | | C1002 | Male | Whole | 10.53 | 4.35 | 10.7 | 35.1 | 80.7 | 24.5 | 30.4 | 13.6 | 819 | 9.2 | 19.4 |
| | | | | | | Blood | | | | | | | | | | | |
| | | | | C1003 | Male | Whole | 16.4 | 5.91 | 12.4 | 42.1 | 71.2 | 21 | 29.5 | 14.8 | 421 | 9.2 | 43.5 |
| | | | | | | Blood | | | | | | | | | | | |
| 24 h | P3 | 100 | mg/kg | C1001 | Male | Whole | 6.74 | 4.55 | 10.9 | 35.7 | 78.4 | 24.1 | 30.7 | 13.7 | 503 | 8.5 | 39.9 |
| | | | | | | Blood | | | | | | | | | | | |
| | | | | C1002 | Male | Whole | 7.75 | 4.22 | 10.2 | 33.9 | 80.5 | 24.2 | 30.1 | 13.5 | 806 | 8.9 | 15.7 |
| | | | | | | Blood | | | | | | | | | | | |
| | | | | C1003 | Male | Whole | 12.14 | 5.49 | 11.6 | 38.8 | 70.6 | 21.1 | 29.9 | 14.9 | 401 | 9 | 34.1 |
| | | | | | | Blood | | | | | | | | | | | |

| | | | | Repeat | Sex/QC | Sample | % | % MONO | % EOS | % BASO | #NEUT\hat{ }3/ |
|---|---|---|---|---|---|---|---|---|---|---|---|
| Time | Phase | Dose level | | No. | Identifier | Type | LYMP | % | % | % | μL |
| pre- | P1 | 2.5 | mg/kg | C1001 | Male | Whole | 72.2 | 4.2 | 4.8 | 0.3 | 1.5 |
| dose | | | | | | Blood | | | | | |
| | | | | C1002 | Male | Whole | 80 | 5.5 | 1.6 | 0.6 | 0.79 |
| | | | | | | Blood | | | | | |
| | | | | C1003 | Male | Whole | 73.4 | 5.2 | 3.4 | 0.4 | 2.33 |
| | | | | | | Blood | | | | | |
| 24 h | P1 | 2.5 | mg/kg | C1001 | Male | Whole | 38.2 | 2.1 | 1.5 | 0.2 | 5.46 |
| | | | | | | Blood | | | | | |
| | | | | C1002 | Male | Whole | 74.1 | 4.9 | 0.9 | 0.5 | 1.08 |
| | | | | | | Blood | | | | | |
| | | | | C1003 | Male | Whole | 58.8 | 2.9 | 2 | 0.2 | 4.13 |
| | | | | | | Blood | | | | | |
| pre- | P2 | 25 | mg/kg | C1001 | Male | Whole | 67.7 | 3.4 | 2.2 | 0.3 | 1.84 |
| dose | | | | | | Blood | | | | | |
| | | | | C1002 | Male | Whole | 74.1 | 3.3 | 1.1 | 0.3 | 1.85 |
| | | | | | | Blood | | | | | |
| | | | | C1003 | Male | Whole | 63.9 | 3.3 | 3.2 | 0.2 | 4.17 |
| | | | | | | Blood | | | | | |
| 24 h | P2 | 25 | mg/kg | C1001 | Male | Whole | 69 | 4.6 | 1.3 | 0.2 | 1.41 |
| | | | | | | Blood | | | | | |
| | | | | C1002 | Male | Whole | 87.4 | 2.8 | 0.6 | 0.4 | 0.52 |
| | | | | | | Blood | | | | | |
| | | | | C1003 | Male | Whole | 70.6 | 3.3 | 3.2 | 0.3 | 2.17 |
| | | | | | | Blood | | | | | |
| pre- | P3 | 100 | mg/kg | C1001 | Male | Whole | 59.1 | 4.4 | 2.3 | 0.1 | 3.13 |
| dose | | | | | | Blood | | | | | |
| | | | | C1002 | Male | Whole | 72 | 5 | 1.4 | 0.3 | 2.05 |
| | | | | | | Blood | | | | | |
| | | | | C1003 | Male | Whole | 48.4 | 3.7 | 3.4 | 0.2 | 7.13 |
| | | | | | | Blood | | | | | |
| 24 h | P3 | 100 | mg/kg | C1001 | Male | Whole | 53.5 | 4.1 | 1.3 | 0.2 | 2.69 |
| | | | | | | Blood | | | | | |
| | | | | C1002 | Male | Whole | 76.6 | 4.7 | 0.9 | 0.3 | 1.22 |
| | | | | | | Blood | | | | | |
| | | | | C1003 | Male | Whole | 57.7 | 57.7 | 3.8 | 0.3 | 4.14 |
| | | | | | | Blood | | | | | |

| | | | | Repeat | #LYMP\hat{ }3/ | #MONO\hat{ }3/ | #EOS\hat{ }3/ | #BASO\hat{ }3/ | % RET | #RET10\hat{ }9/ |
|---|---|---|---|---|---|---|---|---|---|---|
| Time | Phase | Dose level | | No. | μL | μL | μL | μL | % | L |
| pre- | P1 | 2.5 | mg/kg | C1001 | 6.08 | 0.35 | 0.4 | 0.03 | 4.34 | 240.7 |
| dose | | | | C1002 | 6.18 | 0.43 | 0.12 | 0.04 | 4.03 | 189.4 |
| | | | | C1003 | 10.58 | 0.75 | 0.48 | 0.05 | 4.3 | 242.2 |
| 24 h | P1 | 2.5 | mg/kg | C1001 | 3.64 | 0.2 | 0.15 | 0.02 | 4.07 | 209.6 |
| | | | | C1002 | 4.69 | 0.31 | 0.06 | 0.03 | 3.4 | 146.3 |
| | | | | C1003 | 6.93 | 0.35 | 0.24 | 0.03 | 3.96 | 213.1 |
| pre- | P2 | 25 | mg/kg | C1001 | 4.84 | 0.25 | 0.16 | 0.02 | 3.83 | 204.2 |
| dose | | | | C1002 | 7.03 | 0.32 | 0.1 | 0.03 | 3.91 | 173.3 |
| | | | | C1003 | 9.38 | 0.49 | 0.47 | 0.03 | 3.63 | 206.6 |
| 24 h | P2 | 25 | mg/kg | C1001 | 4.07 | 0.27 | 0.08 | 0.01 | 3.7 | 185.9 |
| | | | | C1002 | 6.45 | 0.21 | 0.04 | 0.03 | 5.11 | 227.8 |
| | | | | C1003 | 7.21 | 0.34 | 0.33 | 0.03 | 3.89 | 222.5 |
| pre- | P3 | 100 | mg/kg | C1001 | 5.55 | 0.42 | 0.21 | 0.01 | 3.72 | 181.4 |
| dose | | | | C1002 | 7.58 | 0.53 | 0.15 | 0.03 | 4.95 | 215.3 |
| | | | | C1003 | 7.94 | 0.6 | 0.56 | 0.04 | 3.64 | 215.4 |
| 24 h | P3 | 100 | mg/kg | C1001 | 3.61 | 0.28 | .09 | 0.01 | 3.72 | 169 |
| | | | | C1002 | 5.94 | 0.36 | .07 | 0.02 | 3.98 | 167.8 |
| | | | | C1003 | 7 | 0.38 | 0.46 | 0.03 | 3.37 | 184.9 |

After IV bolus administration at 2.5 mg/kg, concentrations of Compound 2 declined with a mean half-life at 3.95±0.588 hours and a plasma clearance (CL) of 5.17±0.866 mL/min/kg. The volume of distribution (Vdₛₛ) was 1.32±0.0682 L/kg and the area under the plasma concentration time curve from time zero to the last quantifiable concentration (AUC₀₋ₗₐₛₜ) values was 8078±1199 ng·h/mL. Oral administration of 25 mg/kg of Compound 2 was absorbed with a Cₘₐₓ value of 14200±3637 ng/mL at a Tₘₐₓ of 2.67±1.15 hours. The AUC₀₋ₗₐₛₜ was 151182±58128 ng·h/mL after oral administration of 25 mg/kg of Compound 2. Oral administration of 100 mg/kg of Compound 2 was absorbed with a Cₘₐₓ value of 56033±29561 ng/mL at a Tₘₐₓ of 6.67±2.31 hours. The AUC₀₋ₗₐₛₜ was 1007009±327373 ng·h/mL after oral administration of 100 mg/kg of Compound 2. The mean percent oral bioavailability was greater than 100% at both oral doses in male Cynomolgus monkeys. Both oral doses resulted in greater than 100% bioavailability, likely due to the significant difference in dose levels between IV and PO administrations.

After IV bolus administration at 2.5 mg/kg, concentrations of Compound 2 declined with a mean half-life at 3.95±0.588 h and a plasma clearance (CL) of 5.17±0.866 mL/min/kg. The volume of distribution (Vdₛₛ) was 1.32±0.0682 L/kg, and the area under the plasma concentration-time curve from time zero to the last quantifiable concentration (AUC₀₋ₗₐₛₜ) values was 8078±1199 ng·h/mL. TABLE 106 shows plasma PK data of Compound 2 following single IV bolus and oral administrations to non-naïve male cynomolgus monkeys.

TABLE 106

| | Phase | | | | | |
|---|---|---|---|---|---|---|
| | 1 | | 2 | | 3 | |
| | Dose Route | | | | | |
| | IV bolus | | PO | | | |
| | Dose Level (mg/kg) | | | | | |
| | 2.5 | | 25 | | 100 | |
| Parameter | Mean | SD | Mean | SD | Mean | SD |
| C₀ or Cₘₐₓ (ng/mL) | 2823 | 571 | 14200 | 3637 | 56033 | 29561 |
| Tₘₐₓ (h) | —¹ | — | 2.67 | 1.15 | 6.67 | 2.31 |
| T_{1/2} (h) | 3.95 | 0.588 | 7.14 | 0.517 | 18.0 | 11.2 |
| Vdₛₛ (L/kg) | 1.32 | 0.0682 | — | — | — | — |
| C_{L} (mL/min/kg) | 5.17 | 0.866 | — | — | — | — |
| AUC₀₋ₗₐₛₜ (ng · h/mL) | 8078 | 1199 | 151182 | 58128 | 1007009 | 327373 |
| AUC_{0-inf} (ng · h/mL) | 8203 | 1255 | 151406 | 58256 | 1110757 | 411099 |
| Bioavailability (%)² | — | — | 182 | 53.1 | 330 | 81.6 |

¹“—” means not calculated.
²Bioavailability (%) was calculated using AUC_{0-inf} (% AUC_{Extra} < 20%) or AUC₀₋ₗₐₛₜ (% AUC_{Extra} > 20%) with nominal dose.

### Example 19: BSEP Inhibition Assessment Using BSEP-Expressing Vesicles

The BSEP inhibition potential of Compound 1 and Compound 2 were determined using BSEP-expressing vesicles. An assay to measure BSEP-mediated TCA uptake into inside-out BSEP-expressing vesicles was used. BSEP vesicles (50 μg) and TCA (1 μM) were incubated in the absence and presence of the test article (10 μM) or the positive control inhibitor CsA (10 μM). The reaction was initiated by adding 5 mM ATP (adenosine 5′-triphosphate) as the energy source for BSEP. Negative controls were run in parallel, using 5 mM AMP (adenosine 5′-monophosphate) in place of ATP. The reactions were carried out in 96-well plates incubated in a humidified incubator (37° C. 5% CO2) for 30 minutes. After incubation, the vesicle-associated TCA and free TCA were separated by rapid filtration through a glass-fiber filter plate under vacuum.

All samples were assayed by liquid chromatography-tandem mass spectrometry (LC-MS/MS) using electrospray ionization. Liquid Chromatography was performed using a Thermo BDS Hypersil C18 30×2.1 mm, 3 μm, with guard column. The M.P Buffer was 25 mM ammonium formate buffer, pH 3.5. The Aqueous Reservoir (A) was 90% water, 10% buffer. The Organic Reservoir (B) was 90% acetonitrile, 10% buffer. The Flow Rate was 700 μL/minute. The Total Run Time for the liquid chromatography was 2.5 minutes. The Autosampler was 20 μL injection volume. The Autosampler Wash was water/methanol/2-propanol: 1/1/1; with 0.2% formic acid. Mass Spectrometry was performed on the PE SCIEX API 4000 instrument. The interface was Turbo Ionspray. The mode was Multiple reaction monitoring. The method was performed over 2.5 minute duration.

BSEP Inhibition Classification for compound 1 and compound 2 was negative. TABLE 107 summarizes the results of the BSEP inhibition assay.

TABLE 107

| | | | | | | | | BSEP |
|---|---|---|---|---|---|---|---|---|
| | TCA Concentration (nM) | | | | | | | Inhibition |
| | Cofactor ATP | | | Cofactor AMP | | | Percent | Potential |
| Treatment | R1 | R2 | AVG | R1 | R2 | AVG | Inhibition^{a} | Classification |
| TCA | 75. | 82.3 | 78.9 | 1.53 | 1.03 | 1.28 | — | — |
| TCA + 10 μM | 86. | 68.6 | 77.5 | 2.56 | 2.46 | 2.51 | 3.39 | Negative |
| Compound 2 | | | | | | | | |
| TCA + 10 μM | 87. | 83.2 | 85.5 | 1.50 | 2.44 | 1.97 | 0 | Negative |
| Compound 1 | | | | | | | | |
| TCA + 10 μM | 14. | 10.9 | 12.5 | 1.96 | 1.15 | 1.56 | 86.0 | Positive |
| CsA | | | | | | | | |

^{a}Percent inhibition values calculated as negative are reported as 0.
Inhibition Potential Classification:
Percent inhibition ≥50%: Positive
Percent inhibition <50%: Negative

### Example 20: Evaluation of the Potential for Induction of Cytochrome P450 Enzymes in Human Hepatocytes by Compound 2

The cytotoxicity and induction of mRNA expression and enzyme activity of cytochrome P450 (CYP) 1A2, 2B6, and 3A in human hepatocytes by Compound 2 was evaluated in human hepatocytes. Stock solutions of up to 100 mM were prepared in dimethyl sulfoxide (DMSO) and diluted into cell culture medium for the induction treatment. The chemicals and reagents used in the assay, including CYP probe substrates, metabolites, and positive and negative inducers are shown in TABLE 108. All other chemicals and reagents were of analytical grade or higher.

TABLE 108

| | Probe | | Positive | Negative |
|---|---|---|---|---|
| CYP | Substrate | Metabolite | Inducer | Inducer |
| CYP1A2 | Phenacetin | Acetaminophen | Omeprazole (OME) | Flumazenil |
| | 100 μM | | 50 μM | 10 μM |
| CYP2B6 | Bupropion | Hydroxybupropion | Phenobarbital (PB) | Flumazenil |
| | 250 μM | | 1000 μM | 10 μM |
| CYP3A | Midazolam | 1′- | Rifampicin (RIF) | Flumazenil |
| | 20 μM | Hydroxymidazolam | 50 μM | 10 μM |

Human hepatocytes: Plateable and inducible cryopreserved human hepatocytes were purchased. The human hepatocytes for cytotoxicity were obtained from a 28 year old Asian male. The human hepatocytes for CYP induction were obtained from three donors: one 60 year old Caucasian female; one 57 year old Caucasian male; and one 52 year old Caucasian female.

Cytotoxicity of Compound 2 in Human Hepatocytes: Plateable and inducible cryopreserved human hepatocytes were thawed and isolated in human hepatocyte thawing medium. The cells were suspended in human hepatocyte plating medium, counted (cell viability assessed by Trypan blue exclusion), seeded (Day 0) onto collagen-coated 48-well plates at 0.75 million cells/mL (0.15 million cells/well in a 48-well plate), and incubated in a 95% air/5% CO₂ incubator at 37° C. After attachment (4 hours), the medium was changed to fresh hepatocyte culture medium for overnight cell recovery. Hepatocytes were then treated with hepatocyte culture medium fortified with the Compound 2 at five concentrations (1, 5, 10, 50, and 100 μM). A positive control (100 μM chlorpromazine) was treated in parallel. Vehicle controls were treated with hepatocyte culture medium containing the same content of organic solvent (0.1% DMSO). The hepatocyte incubation was conducted in a 95% air/5% CO₂ incubator at 37° C. for three days (72 hours) with daily replacement of the hepatocyte culture medium containing Compound 2, positive control, or vehicle. The viability of cells was measured by analyzing the cellular conversion of tetrazolium compound [3-(4,5-dimethylthiazol-2-yl)-5-(3-carboxymethoxyphenyl)-2-(4-sulfophenyl)-2H tetrazolium, inner salt; MTS] into a formazan product by dehydrogenases, which are active only in viable cells. The absorbance of formazan, which is proportional to the number of viable cells, was measured spectrophotometrically using the CellTiter 96® AQueous One Solution Cell Proliferation Assay (MTS). The wells were rinsed with DPBS, and then hepatocyte culture medium (200 μL) and the CellTiter 96® AQueous One Solution Cell Proliferation Assay reagent (40 μL) were added to each well. The cells were then incubated for 1 hour at 37° C. in a 95% air/5% CO₂ incubator. The absorbance of formazan in each well was measured at 492 nm using a FLUOstar® OPTIMA Microplate Reader.

CYP Induction of Compound 2: Plateable and inducible cryopreserved human hepatocytes were thawed and isolated in human hepatocyte thawing medium. The cells were suspended in human hepatocyte plating medium, counted (cell viability assessed by Trypan blue exclusion), seeded (Day 0) onto collagen-coated 48-well plates at 0.75 million cells/mL (0.15 million cells/well in a 48-well plate), and incubated in a 95% air/5% CO₂ incubator at 37° C. After attachment (4 hours), the medium was changed to fresh hepatocyte culture medium for overnight cell recovery. Hepatocytes were then treated with hepatocyte culture medium fortified with the Compound 2 at three concentrations (5 μM, 10 μM, and 20 μM, based on the cytotoxicity test results). Positive controls were treated in parallel with hepatocyte culture medium fortified with a known inducer of each CYP of interest: 50 μM OME for CYP1A2, 1,000 μM PB for CYP2B6, or 50 μM RIF for CYP3A. Negative controls were treated with 10 μM flumazenil, and vehicle controls were treated with hepatocyte culture medium containing 0.1% DMSO. All experiments were performed in triplicate. The hepatocyte incubation was conducted in a 95% air/5% CO₂ incubator at 37° C. for three days (72 hours) with daily replacement of the hepatocyte culture medium containing Compound 2, positive or negative inducer, or vehicle. CYP Enzyme Activity Assay: CYP enzyme activity was determined by measuring the formation of a CYP probe substrate metabolite. The wells were washed with DPBS and incubated with 200 μL of WME containing an individual CYP probe substrate at 37° C. for 1 hour in a 95% air/5% CO₂ incubator. After the incubation, 150 μL of the CYP incubation mixture from each well was transferred into a 96-well plate containing 300 μL of ice-cold acetonitrile and an internal standard (IS, stable isotope-labeled CYP probe metabolite) per well. The solutions were mixed and centrifuged at 1,640 g for 10 minutes. The supernatants were transferred to an HPLC autosampler plate, and the concentrations of CYP probe metabolite were analyzed by LC-MS/MS.

Cell Viability Assay: After the CYP induction treatment and enzyme activity assay, the cells were used for a cell viability assay. The viability of cells, expressed as the percentage of MTS absorbance relative to vehicle control, was measured as described above.

qPCR-mRNA Assay: Total RNA was isolated from the treated cells using the RNeasy® mini kit and treated with RNase-free DNase. The concentration of RNA was determined using a Qubit® Fluorometer with a Qubit® RNA HS assay kit. cDNA was synthesized from up to 1 μg of the total RNA harvested from the cells using a QuantiTect® RT kit. Analysis of CYP gene expression by qPCR was performed using the LightCycler® 480 II System. Primer and Probe sequences used for the assay are shown in TABLE 109.

TABLE 109

| | Forward | Reverse | |
|---|---|---|---|
| Gene | Primer | Primer | Probe |
| CYP1A2 | ccagctgcc | gtgtccct | cctggaga |
| | ctacttgga | tgttgtgc | (SEQ ID |
| | (SEQ ID | tgtg | NO: 4) |
| | NO: 2) | (SEQ ID | |
| | | NO: 3) | |
| CYP2B6 | acttcggga | gaggaag | tggaggag |
| | tgggaaagc | gtggggt | (SEQ ID |
| | (SEQ ID | ccat | NO: 7) |
| | NO: 5) | (SEQ ID | |
| | | NO: 6) | |
| CYP3A4 | gatggctct | agtccat | ttctcctg |
| | catcccaga | gtgaatg | (SEQ ID |
| | ctt | ggttcc | NO: 10) |
| | (SEQ ID | (SEQ ID | |
| | NO: 8) | NO: 9) | |

Disappearance of Compound 2 After Induction Treatment: On Day 3 of treatment of human hepatocytes, the disappearance of Compound 2 in the hepatocyte culture medium, at 0 (dosing solution), 4, 8, and 24 hours post-dose, was measured by LC-MS/MS using the method described below.

Determination of Compound 2: Liquid Chromatography was performed using a Waters ACQUITY UPLC BEH Phenyl 30×2.1 mm, 1.7 μm column; 25 mM ammonium formate (pH 3.5) buffer; 90% water/10% buffer Mobile Phase A; 90% ACN/10% buffer Mobile Phase B; and a 0.7 mL/minute flow rate. The gradient program is shown in TABLE 110.

TABLE 110

| Time (min) | % A | % B |
|---|---|---|
| 0.00 | 99 | 1 |
| 0.65 | 1 | 99 |
| 0.75 | 1 | 99 |
| 0.80 | 99 | 1 |
| 1.00 | 99 | 1 |

Run time: 1.0 minute
Injection volume: 5 μL
Autosampler Wash: water/methanol/2-propanol: 1/1/1 (v/v/v); with 0.2% formic acid

Mass Spectrometry was performed using a PE SCIEX API 4000; Turbo Ionspray interface; and Multiple Reaction Monitoring (MRM) quantification. TABLE 111 shows the MS parameter settings to detect Compound 2. Abbreviations—DP: declustering potential; EP: entrance potential; CE: collision energy; CXP: Collision cell exit potential; ISV: ion spray voltage; TEM: ion source temperature; CAD: collisionally activated dissociation; CUR: curtain gas.

TABLE 111

| Analyte | Q1/Q3 | DP | EP | CE | CXP | ISV | TEM | CAD | CUR | GS1 | GS2 |
|---|---|---|---|---|---|---|---|---|---|---|---|
| Compound 2 | +546/114 | 100 | 10 | 35 | 11 | 5500 | 500 | 7 | 30 | 50 | 50 |
| Metoprolol (IS) | +268/116 | 70 | 10 | 26 | 10 | 5500 | 500 | 7 | 30 | 50 | 50 |

Calibration Curve: A calibration curve for the quantification of Compound 2 was prepared by fortifying a standard solution of Compound 2 into blank hepatocyte culture medium at six to eight concentrations. The fortified standards were treated by the addition of protein precipitation solvent. After centrifugation at 1,640 g (3,000 rpm) for 10 minutes, the supernatants were analyzed by LC-MS/MS. The acceptance criterion for the calibration curve was at least 75% of standards within 85.0% to 115% accuracy except at the LLOQ, where 80.0% to 120% accuracy was acceptable.

Determination of CYP Probe Metabolites: Liquid Chromatography was performed using a Thermo Hypersil BDS C18 30×2.1 mm i.d., 3 μm, column with guard cartridge; 25 mM ammonium formate (pH 3.5) buffer; 90% water/10% buffer Mobile Phase A; 90% ACN/10% buffer Mobile Phase B; and a 0.3-0.35 mL/minute flow rate. TABLE 112 shows the gradient program used to determine acetaminophen (CYP1A2). TABLE 113 shows the gradient program used to determine OH bupropioin and 1′-OH Midazolam (CYP2B6 and CYP3A).

TABLE 112

| Time (min) | % A | % B |
|---|---|---|
| 0.0 | 100 | 0 |
| 0.6 | 70 | 30 |
| 2.0 | 0 | 100 |
| 2.1 | 100 | 0 |
| 3.0 | 100 | 0 |

TABLE 113

| Time (min) | % A | % B |
|---|---|---|
| 0.0 | 100 | 0 |
| 0.8 | 20 | 80 |
| 1.0 | 0 | 100 |
| 1.5 | 0 | 100 |
| 1.6 | 100 | 0 |
| 2.5 | 100 | 0 |

Mass Spectrometry was performed using a PE SCIEX API 4000; a Turbo Ionspray interface; Multiple Reaction Monitoring (MRM) quantification. The MS parameter settings are shown in TABLE 114. Abbreviations—DP: declustering potential; EP: entrance potential; CE: collision energy; CXP: Collision cell exit potential; ISV: ion spray voltage; TEM: ion source temperature; CAD: collisionally activated dissociation; CUR: curtain gas.

TABLE 114

| Analyte | Q1/Q3 | DP | EP | CE | CXP | ISV | TEM | CAD | CUR | GS1 | GS2 |
|---|---|---|---|---|---|---|---|---|---|---|---|
| Acetaminophen | +152/110 | 78 | 10 | 23 | 6 | 5500 | 500 | 7 | 20 | 20 | 30 |
| Acetaminophen-²H₄ | +156/114 | 63 | 10 | 23 | 11 | 5500 | 500 | 7 | 20 | 20 | 30 |
| OH bupropion | +256/139 | 43 | 10 | 30 | 9 | 5500 | 500 | 7 | 20 | 20 | 30 |
| OH Buproprion-²H₆ | +262/139 | 53 | 10 | 33 | 9 | 5500 | 500 | 7 | 20 | 20 | 30 |
| 1′-OH midazolam | +342/203 | 44 | 10 | 30 | 17 | 5500 | 500 | 7 | 20 | 20 | 30 |
| 1′-OH midazolam-¹³C₃ | +345/206 | 96 | 10 | 38 | 12 | 5500 | 500 | 7 | 20 | 20 | 30 |

Calibration Curves: Calibration curves for the quantification of the CYP probe metabolites were prepared by fortifying standard solutions of each metabolite into blank hepatocyte culture medium at six to eight concentrations. The fortified standards were treated by the addition of protein precipitation solvent. After centrifugation at 1,640 g (3,000 rpm) for 10 minutes, the supernatants were analyzed by LC MS/MS. The acceptance criterion for the calibration curves was at least 75% of standards within 85.0% to 115% accuracy except at the LLOQ, where 80.0% to 120% accuracy was acceptable.

Data Analysis: CYP enzyme activity was expressed as the formation rate of CYP probe metabolite, normalized to the cell viability (MTS absorbance): Normalized Activity=Formation rate of CYP probe metabolite/MTS absorbance. The CYP enzyme activity in cells treated with Compound 2 was compared to the activity in cells treated with vehicle and positive controls using the following equations: Fold-change relative to vehicle control=Activity_{TA}/Activity_{vehicle} % Induction relative to positive control=100×(Activity_{TA}−Activity_{vehicle})/(Activity_{positive control}−Activity_{vehicle}). Relative mRNA was expressed as the fold-change calculated from the normalized mRNA level (2−ΔΔCt) relative to the vehicle control. The percentage of mRNA fold-change relative to the positive control was calculated using the following equation: % Induction relative to positive control=100×(mRNA_{TA}−mRNA_{vehicle})/(mRNA_{positive control}−mRNA_{vehicle}).

Cytotoxicity: The cytotoxicity, expressed as cell viability, the percentage of MTS absorbance relative to the vehicle control, of Compound 2 and the positive control (100 μM chlorpromazine) in human hepatocytes is summarized in TABLE 115.

TABLE 115

| | | | | % MTS |
|---|---|---|---|---|
| | | Formazan | | Absorbance |
| | | Absorbance | | Relative |
| | | (n = 6) | | to Vehicle |
| Test Article | Treatment | Mean | SD | Control^{a} |
| Control | Vehicle | 0.293 | 0.00909 | 100 |
| | Chlorpromazine | 0.174 | 0.00207 | 59.4 |
| Compound 2 | 100 μM | 0.172 | 0.00337 | 58.7 |
| | 50 μM | 0.168 | 0.00387 | 57.3 |
| | 10 μM | 0.269 | 0.0109 | 91.7 |
| | 5 μM | 0.299 | 0.0197 | 102 |
| | 1 μM | 0.288 | 0.0115 | 98.2 |

^{a}MTS absorbance <75% of the vehicle control is considered a positive cytotoxic result.

CYP Induction: TABLE 116 shows induction of CYP1A2 mRNA in human hepatocytes by Compound 2, positive controls, and negative controls. TABLE 117 shows induction of CYP1A2 enzyme activity in human hepatocytes by Compound 2, positive controls, and negative controls. TABLE 118 shows cell viability results after CYP1A2 induction treatment in human hepatocytes with Compound 2, positive controls, and negative controls. TABLE 119 shows induction of CYP2B6 mRNA in human hepatocytes by Compound 2, positive controls, and negative controls. TABLE 120 shows induction of CYP2B6 enzyme activity in human hepatocytes by Compound 2, positive controls, and negative controls. TABLE 121 shows cell viability results after CYP2B6 induction treatment in human hepatocytes with Compound 2, positive controls, and negative controls. TABLE 122 shows induction of CYP3A mRNA in human hepatocytes by Compound 2, positive controls, and negative controls. TABLE 123 shows induction of CYP3A enzyme activity in human hepatocytes by Compound 2, positive controls, and negative controls. TABLE 124 shows cell viability results after CYP3A induction treatment in human hepatocytes with Compound 2, positive controls, and negative controls.

TABLE 125 shows measured concentrations of Compound 2 in culture medium on Day 3 of treatment of human Hepatocytes (Donor 1). TABLE 126 shows measured concentrations of Compound 2 in culture medium of Day 3 of treatment of human hepatocytes (Donor 2). TABLE 127 shows measured concentrations of Compound 2 in culture medium on Day 3 of treatment of human Hepatocytes (Donor 3). The data show the disappearance of Compound 2 in the hepatocyte culture medium on Day 3 of treatment of human hepatocytes.

TABLE 116

| | | | Fold | | % Induction |
|---|---|---|---|---|---|
| | | | Change^{a} | | Relative |
| | Test | | (n = 3) | | to Positive |
| Donor | Article | Treatment | Mean | SD | Control^{b} |
| 1 | Control | Vehicle | 1.00 | 0.0733 | 0 |
| | | OME | 126 | 18.6 | 100 |
| | | Flumazenil | 0.760 | 0.158 | 0 (−0.194) |
| | Compound 2 | 20 μM | 0.569 | 0.106 | 0 (−0.346) |
| | | 10 μM | 1.29 | 0.203 | 0.232 |
| | | 5 μM | 0.881 | 0.0772 | 1 (−0.0967) |
| 2 | Control | Vehicle | 1.01 | 0.176 | 0 |
| | | OME | 12.0 | 3.38 | 100 |
| | | Flumazenil | 0.909 | 0.201 | 0 (−0.920) |
| | Compound 2 | 20 μM | 4.13 | 0.909 | 28.3 |
| | | 10 μM | 2.81 | 0.235 | 16.3 |
| | | 5 μM | 2.34 | 1.18 | 12.0 |
| 3 | Control | Vehicle | 1.20 | 0.795 | 0 |
| | | OME | 43.0 | 3.27 | 100 |
| | | Flumazenil | 0.218 | 0.0218 | 0 (−2.35) |
| | Compound 2 | 20 μM | 0.527 | 0.134 | 0 (−1.61) |
| | | 10 μM | 0.819 | 0.908 | 0 (−0.913) |
| | | 5 μM | 0.237 | 0.0497 | 0 (−2.30) |

^{a}Fold-increase was calculated from the normalized mRNA level (2^{-ΔΔCt}) of TA-, positive inducer-, or negative inducer-treated cells relative to that of vehicle-treated cells.
^{b}Percentage of mRNA fold-increase relative to positive inducer-treated cells. Negative values are treated as zero.

TABLE 117

| | | | | Acetaminophen | | | % Induction |
|---|---|---|---|---|---|---|---|
| | | | | Formation | | | Relative to |
| | | | | (pmol/well/min. n = 3 | | Fold- | Positive |
| Donor | Test Article | Treatment | | Mean | SD | Change^{a} | Control^{b} |
| 1 | Control | Vehicle | | 1.78 | 0.308 | 1.00 | 0 |
| | | OME | | 87.5 | 20.4 | 49.1 | 100 |
| | | Flumazenil | | 1.85 | 0.207 | 1.04 | 0.0732 |
| | Compound 2 | 20 | μM | 2.15 | 0.255 | 1.21 | 0.428 |
| | | 10 | μM | 2.90 | 0.0262 | 1.62 | 1.30 |
| | | 5 | μM | 2.24 | 0.172 | 1.26 | 0.535 |
| 2 | Control | Vehicle | | 0.224 | 0.0219 | 1.00 | 0 |
| | | OME | | 1.41 | 0.165 | 6.31 | 100 |
| | | Flumazenil | | 0.197 | 0.0124 | 0.880 | 0 (−2.26) |
| | Compound 2 | 20 | μM | 0.179 | 0.0397 | 0.803 | 0 (−3.72) |
| | | 10 | μM | 1.30 | 0.272 | 5.81 | 90.7 |
| | | 5 | μM | 0.998 | 0.127 | 4.46 | 65.2 |
| 3 | Control | Vehicle | | 0.411 | 0.0442 | 1.00 | 0 |
| | | OME | | 8.41 | 0.480 | 20.5 | 100 |
| | | Flumazenil | | 0.330 | 0.0522 | 0.804 | 0 (−1.00) |
| | Compound 2 | 20 | μM | 0.264 | 0.0855 | 0.642 | 0 (−1.84) |
| | | 10 | μM | 1.33 | 0.444 | 3.24 | 11.5 |
| | | 5 | μM | 0.679 | 0.114 | 1.65 | 3.35 |

TABLE 118

| | | | | | % MTS |
|---|---|---|---|---|---|
| | | | Formazan | | Absorbance |
| | | | Absorbance | | Relative |
| | Test | | (n = 3) | | to Vehicle |
| Donor | Article | Treatment | Mean | SD | Control |
| 1 | Control | Vehicle | 0.488 | 0.0361 | 100 |
| | | OME | 0.494 | 0.0178 | 101 |
| | | Flumazenil | 0.489 | 0.0146 | 100 |
| | Compound 2 | 20 μM | 0.665 | 0.0164 | 136 |
| | | 10 μM | 0.640 | 0.0172 | 131 |
| | | 5 μM | 0.491 | 0.00400 | 101 |
| 2 | Control | Vehicle | 0.654 | 0.0521 | 100 |
| | | OME | 0.800 | 0.0291 | 122 |
| | | Flumazenil | 0.642 | 0.0202 | 98.2 |
| | Compound 2 | 20 μM | 0.382 | 0.104 | 58.4^{a} |
| | | 10 μM | 0.728 | 0.106 | 111 |
| | | 5 μM | 0.678 | 0.0616 | 104 |
| 3 | Control | Vehicle | 0.414 | 0.0119 | 100 |
| | | OME | 0.433 | 0.0300 | 104 |
| | | Flumazenil | 0.396 | 0.00681 | 95.5 |
| | Compound 2 | 20 μM | 0.243 | 0.00306 | 58.7^{a} |
| | | 10 μM | 0.473 | 0.0502 | 114 |
| | | 5 μM | 0.434 | 0.0351 | 105 |

^{a}MTS absorbance <75% of the vehicle control is considered a positive cytotoxic result.

TABLE 119

| | | | Fold | | % Induction |
|---|---|---|---|---|---|
| | | | Change^{a} | | Relative |
| | Test | | (n = 3) | | to Positive |
| Donor | Article | Treatment | Mean | SD | Control^{b} |
| 1 | Control | Vehicle | 1.03 | 0.336 | 0 |
| | | PB | 24.4 | 5.43 | 100 |
| | | Flumazenil | 0.852 | 0.0911 | 0 (0.776) |
| | Compound 2 | 20 μM | 0.916 | 0.743 | 0 (−0.503) |
| | | 10 μM | 0.786 | 0.163 | 0 (−1.06) |
| | | 5 μM | 0.977 | 0.305 | 0 (−0.241) |
| 2 | Control | Vehicle | 1.00 | 0.0801 | 0 |
| | | PB | 793 | 278 | 100 |
| | | Flumazenil | 1.01 | 0.301 | 0.00144 |
| | Compound 2 | 20 μM | 0.597 | 0.737 | 0 (−0.0512) |
| | | 10 μM | 5.68 | 0.482 | 0.591 |
| | | 5 μM | 5.50 | 1.11 | 0.568 |
| 3 | Control | Vehicle | 1.00 | 0.0325 | 0 |
| | | PB | 6.85 | 1.71 | 100 |
| | | Flumazenil | 0.530 | 0.564 | 0 (−8.05) |
| | Compound 2 | 20 μM | 0.0958 | NA | 0 (−15.5) |
| | | 10 μM | 0.930 | 0.244 | 0 (−10.4) |
| | | 5 μM | 0.664 | 0.447 | 0 (−5.76) |

^{a}Fold-increase was calculated from the normalized mRNA level (2^{-ΔΔCr}) of TA-positive inducer-, or negative inducer-treated cells relative to that of vehicle-treated cells.
^{b}Percentage of mRNA fold-increase relative to that of vehicle-treated cells.
Negative values are treated as zero.
NA not applicable, n = 2.

TABLE 120

| | | | | OH Bupropion | | | % Induction |
|---|---|---|---|---|---|---|---|
| | | | | Formation | | | Relative to |
| | | | | (pmol/well/min; n = 3) | | Fold- | Positive |
| ΔDonor | Test Article | Treatment | | Mean | SD | Change^{a} | Control^{b} |
| 1 | Control | Vehicle | | 5.79 | 1.11 | 1.00 | 0 |
| | | PB | | 39.6 | 5.09 | 6.84 | 100 |
| | | Flumazenil | | 5.31 | 0.730 | 0.917 | 0 (−1.42) |
| | Compound 2 | 20 | μM | 0.861 | 0.0785 | 0.149 | 0 (−14.6) |
| | | 10 | μM | 2.07 | 0.378 | 0.358 | 0 (−11.0) |
| | | 5 | μM | 4.01 | 0.164 | 0.692 | 0 (−5.27) |
| 2 | Control | Vehicle | | 0.0833 | 0.00534 | 1.00 | 0 |
| | | PB | | 0.712 | 0.0321 | 8.56 | 100 |
| | | Flumazenil | | 0.0734 | 0.00916 | 0.881 | 0 (−1.58) |
| | Compound 2 | 20 | μM | 0 | NA | NA | NA |
| | | 10 | μM | 0.130 | 0.00976 | 1.56 | 7.35 |
| | | 5 | μM | 0.216 | 0.0294 | 2.59 | 21.1 |
| 3 | Control | Vehicle | | 0.507 | 0.112 | 1.00 | 0 |
| | | PB | | 1.70 | 0.223 | 3.36 | 100 |
| | | Flumazenil | | 0.443 | 0.0436 | 0.874 | 0 (−5.35) |
| | Compound 2 | 20 | μM | 0.130 | 0.0268 | 0.256 | 0 (−31.6) |
| | | 10 | μM | 0.276 | 0.0476 | 0.544 | 0 (−19.3) |
| | | 5 | μM | 0.465 | 0.0508 | 0.917 | 0 (−3.52) |

^{a}Fold-increase was calculated from the ratio of normalized enzyme activity (formation rate of probe substrate metabolite) of TA-, positive inducer-, or negative inducer-treated cells to that of vehicle-treated cells.
^{b}Percentage of enzyme activity fold-increase relative to positive inducer-treated cells. Negative values are treated as zero.
NA: not applicable

TABLE 121

| | | | | | % MTS |
|---|---|---|---|---|---|
| | | | Formazan | | Absorbance |
| | | | Absorbance | | Relative |
| | Test | | (n = 3) | | to Vehicle |
| Donor | Article | Treatment | Mean | SD | Control |
| 1 | Control | Vehicle | 0.504 | 0.0620 | 100 |
| | | PB | 0.451 | 0.0598 | 89.5 |
| | | Flumazenil | 0.484 | 0.0635 | 96.0 |
| | Compound 2 | 20 μM | 0.673 | 0.0433 | 133 |
| | | 10 μM | 0.604 | 0.0411 | 120 |
| | | 5 μM | 0.540 | 0.00529 | 107 |
| 2 | Control | Vehicle | 0.696 | 0.0473 | 100 |
| | | PB | 0.672 | 0.0410 | 96.5 |
| | | Flumazenil | 0.677 | 0.0275 | 97.3 |
| | Compound 2 | 20 μM | 0.296 | 0.0340 | 42.6^{a} |
| | | 10 μM | 0.728 | 0.0445 | 104 |
| | | 5 μM | 0.691 | 0.0190 | 99.2 |
| 3 | Control | Vehicle | 0.432 | 0.0189 | 100 |
| | | PB | 0.407 | 0.0286 | 94.2 |
| | | Flumazenil | 0.431 | 0.0542 | 99.8 |
| | Compound 2 | 20 μM | 0.251 | 0.0189 | 58.2^{a} |
| | | 10 μM | 0.506 | 0.0286 | 117 |
| | | 5 μM | 0.454 | 0.0542 | 105 |

^{a}MTS absorbance <75% of the vehicle control is considered a positive cytotoxic result

TABLE 122

| | | | Fold- | | % Induction |
|---|---|---|---|---|---|
| | | | change^{a} | | Relative |
| | Test | | (n = 3) | | to Positive |
| Donor | Article | Treatment | Mean | SD | Control^{b} |
| 1 | Control | Vehicle | 1.03 | 0.292 | 0 |
| | | RIF | 8.16 | 1.32 | 100 |
| | | Flumazenil | 0.543 | 0.0738 | 0 (−6.76) |
| | Compound 2 | 20 μM | 0.442 | 0.111 | 0 (−8.19) |
| | | 10 μM | 1.44 | 0.0904 | 5.79 |
| | | 5 μM | 1.25 | 0.135 | 3.10 |
| 2 | Control | Vehicle | 1.00 | 0.105 | 0 |
| | | RIF | 95.7 | 18.6 | 100 |
| | | Flumazenil | 1.31 | 0.457 | 0.328 |
| | Compound 2 | 20 μM | 11.1 | 4.28 | 10.6 |
| | | 10 μM | 20.3 | 1.21 | 20.4 |
| | | 5 μM | 15.0 | 1.19 | 14.8 |
| 3 | Control | Vehicle | 1.02 | 0.251 | 0 |
| | | RIF | 7.65 | 2.45 | 100 |
| | | Flumazenil | 0.589 | 0.599 | 0 (−6.53) |
| | Compound 2 | 20 μM | 1.15 | 0.142 | 1.93 |
| | | 10 μM | 1.93 | 0.342 | 13.6 |
| | | 5 μM | 1.30 | 0.255 | 4.14 |

^{a}Fold-increase was calculated from the normalized mRNA level (2^{-ΔΔCt}) of TA-, positive inducer- or negative inducer-treated cells relative to that of vehicle-treated cells.
^{b}Percentage of mRNA fold-increase relative to positive inducer-treated cells. Negative values are treated as zero.

TABLE 123

| | | | | 1′-OH Midazolam | | | % Induction |
|---|---|---|---|---|---|---|---|
| | | | | Formation | | | Relative to |
| | | | | (pmol/well/min, n = 3) | | Fold- | Positive |
| Donor | Test Article | Treatment | | Mean | SD | change^{a} | Control^{b} |
| 1 | Control | Vehicle | | 1.55 | 0.368 | 1.00 | 0 |
| | | RIF | | 18.1 | 1.39 | 11.7 | 100 |
| | | Flumazenil | | 1.91 | 0.289 | 1.23 | 2.16 |
| | Compound 2 | 20 | μM | 0.239 | 0.123 | 0.154 | 0 (−7.94) |
| | | 10 | μM | 0.576 | 0.0411 | 0.371 | 0 (−5.90) |
| | | 5 | μM | 0.709 | 0.0862 | 0.456 | 0 (−5.10) |
| 2 | Control | Vehicle | | 0.282 | 0.0316 | 1.00 | 0 |
| | | RIF | | 1.89 | 0.0598 | 6.72 | 100 |
| | | Flumazenil | | 0.261 | 0.00945 | 0.926 | 0 (−1.30) |
| | Compound 2 | 20 | μM | 0.0547 | 0.0142 | 0.194 | 0 (−14.1) |
| | | 10 | μM | 0.144 | 0.00462 | 0.511 | 0 (−8.55) |
| | | 5 | μM | 0.189 | 0.0131 | 0.670 | 0 (−5.77) |
| 3 | Control | Vehicle | | 0.634 | 0.123 | 1.00 | 0 |
| | | RIF | | 4.51 | 1.29 | 7.12 | 100 |
| | | Flumazenil | | 0.667 | 0.0825 | 1.05 | 0.863 |
| | Compound 2 | 20 | μM | 0.0426 | 0.0125 | 0.0673 | 0 (−15.2) |
| | | 10 | μM | 0.185 | 0.0268 | 0.293 | 0 (−11.6) |
| | | 5 | μM | 0.222 | 0.0163 | 0.351 | 0 (−10.6) |

^{a}Fold-increase was calculated from the ratio of normalized enzyme activity (formation rate of probe substrate metabolite) of TA-. positive inducer-, or negative inducer-treated cells to that of vehicle-treated cells.
^{b}Percentage of enzyme activity fold-increase relative positive inducer-treated cells. Negative values are treated as zero.

TABLE 124

| | | | | | % MTS |
|---|---|---|---|---|---|
| | | | Formazan | | Absorbance |
| | | | Absorbance | | Relative |
| | | | (n = 3) | | to Vehicle |
| Donor | Test Article | Treatment | Mean | SD | Control |
| 1 | Control | Vehicle | 0.620 | 0.223 | 100 |
| | | RIF | 0.539 | 0.0521 | 86.9 |
| | | Flumazenil | 0.585 | 0.191 | 94.4 |
| | Compound 2 | 20 μM | 0.515 | 0.159 | 83.1 |
| | | 10 μM | 0.622 | 0.101 | 100 |
| | | 5 μM | 0.624 | 0.143 | 101 |
| 2 | Control | Vehicle | 0.608 | 0.0268 | 100 |
| | | RIF | 0.793 | 0.0442 | 130 |
| | | Flumazenil | 0.664 | 0.00603 | 109 |
| | Compound 2 | 20 μM | 0.349 | 0.0885 | 57.4 |
| | | 10 μM | 0.637 | 0.0270 | 105 |
| | | 5 μM | 0.602 | 0.0267 | 99.1 |
| 3 | Control | Vehicle | 0.413 | 0.00794 | 100 |
| | | RIF | 0.461 | 0.0396 | 112 |
| | | Flumazenil | 0.400 | 0.0148 | 96.9 |
| | Compound 2 | 20 μM | 0.206 | 0.0122 | 49.9′ |
| | | 10 μM | 0.473 | 0.00751 | 115 |
| | | 5 μM | 0.442 | 0.0190 | 107 |

^{a} MTS absorbance <75% of the vehicle control is considered a positive cytotoxic result.

TABLE 125

| Nominal | | 0 hr | 4 hr | 8 hr | 24 hr |
|---|---|---|---|---|---|
| Concentration | Parameter | (dosing | (post- | (post- | (post- |
| (μM) | (n = 1) | solution) | dose) | dose) | dose) |
| 20 | Compound 2 (μM) | 13.20 | 9.03 | 8.76 | 7.47 |
| | % of Nominal | 66.0 | 45.2 | 43.8 | 37.4 |
| | % of 0 hr | 100 | 68.4 | 66.4 | 56.6 |
| 10 | Compound 2 (μM) | 6.71 | 3.78 | 3.36 | 2.71 |
| | % of Nominal | 67.1 | 37.8 | 33.6 | 27.1 |
| | % of 0 hr | 100 | 56.3 | 50.1 | 40.4 |
| 5 | Compound 2 (μM) | 3.18 | 2.08 | 2.12 | 1.47 |
| | % of Nominal | 63.6 | 41.6 | 42.4 | 29.4 |
| | % of 0 hr | 100 | 65.4 | 66.7 | 46.2 |

TABLE 126

| Nominal | | 0 hr | 4 hr | 8 hr | 24 hr |
|---|---|---|---|---|---|
| Concentration | Parameter | (dosing | (post- | (post- | (post- |
| (μM) | (n = 1) | solution) | dose) | dose) | dose) |
| 20 | Compound 2 (μM) | 14.3 | 11.9 | 12.4 | 10.8 |
| | % of Nominal | 71.5 | 59.5 | 62.0 | 54.0 |
| | % of 0 hr | 100 | 83.2 | 86.7 | 75.5 |
| 10 | Compound 2 (μM) | 6.64 | 4.37 | 4.30 | 3.69 |
| | % of Nominal | 66.4 | 43.7 | 43.0 | 36.9 |
| | % of 0 hr | 100 | 65.8 | 64.8 | 55.6 |
| 5 | Compound 2 (μM) | 3.12 | 2.46 | 2.11 | 1.81 |
| | % of Nominal | 62.4 | 49.2 | 42.2 | 36.2 |
| | % of 0 hr | 100 | 78.8 | 67.6 | 58.0 |

TABLE 127

| Nominal | | 0 hr | 4 hr | 8 hr | 24 hr |
|---|---|---|---|---|---|
| Concentration | Parameter | (dosing | (post- | (post- | (post- |
| (μM) | (n = 1) | solution) | dose) | dose) | dose) |
| 20 | Compound 2 (μM) | 14.3 | 14.5 | 13.7 | 10.8 |
| | % of Nominal | 71.5 | 72.5 | 68.5 | 54.0 |
| | % of 0 hr | 100 | 101 | 95.8 | 75.5 |
| 10 | Compound 2 (μM) | 6.49 | 4.76 | 4.00 | 3.68 |
| | % of Nominal | 64.9 | 47.6 | 40.0 | 36.8 |
| | % of 0 hr | 100 | 73.3 | 61.6 | 56.7 |
| 5 | Compound 2 (μM) | 4.33 | 2.92 | 2.51 | 2.17 |
| | % of Nominal | 86.6 | 58.4 | 50.2 | 43.4 |
| | % of 0 hr | 100 | 67.4 | 58.0 | 50.1 |

Compound 2 at 5 μM, 10 μM, and 20 μM did not show induction of either mRNA or enzyme activity of CYP1A2, CYP2B6, or CYP3A4 in two (donor 1 and donor 3) of the three human hepatocyte donors tested. Compound 2 showed relatively higher induction responses of CYP mRNA in donor 2, while the positive controls (particularly for CYP2B6 and CYP3A4) showed very high fold-induction.

Thus, the responses to Compound 2 were not sufficient to define a true positive. Compound 2 did now result in cytotoxicity at 1 μM, 5 μM, or 10 μM, but resulted in cytotoxicity in concentrations above 20 μM. Measurement of the concentrations of Compound 2 in the cell culture medium on Day 3 of treatment of human hepatocytes showed that Compound 2 may have been metabolized by human hepatocytes during the induction treatment.

In donor 1, Compound 2 did not increase either mRNA or enzyme activity of CYP1A2, CYP2B6, or CYP3A4 (<2-fold vs. vehicle control and <20% of positive control) at any of the three tested concentrations. In donor 2, Compound 2 showed 2.34-, 2.81-, and 4.13-fold increases in CYP1A2 mRNA at 5 μM, 10 μM, and 20 μM (12.0%, 16.3%, and 28.3% of the response of the positive control), respectively. Compound 2 showed 4.46-, 5.81-, and 0.803-fold increases in CYP1A2 enzyme activity at 5 μM, 10 μM, and 20 μM (65.2%, 90.7%, and 0 (−3.72%) of the response of the positive control), respectively. Compound 2 showed 5.50-, 5.68-, and 0.597-fold increases in CYP2B6 mRNA at 5 μM, 10 μM, and 20 μM (0.568%, 0.591%, and 0 (−0.0512%) of the response of the positive control), respectively. Compound 2 showed a 2.59-fold increase, 1.56-fold increase, and complete abolition of CYP2B6 enzyme activity at 5 μM, 10 μM, and 20 μM (21.1% and 7.35% of the response of the positive control and not applicable), respectively. Compound 2 showed 15.0-, 20.3-, and 11.1-fold increases of CYP3A4 mRNA at 5 μM, 10 μM, and 20 μM (14.8%, 20.4%, and 10.6% of the response of the positive control), respectively. Compound 2 did not increase CYP3A enzyme activity at any of the three tested concentrations. The decrease of mRNA and/or enzyme activity by Compound 2 at 20 μM is due to cytotoxicity in this donor. Since very high responses of CYP2B6 and CYP3A4 mRNA were observed in this donor for the positive controls (793-fold increase in CYP2B6 mRNA by phenobarbital and 95.7-fold increase in CYP3A4 mRNA by rifampicin), in most cases the results for the test article were below the threshold of a positive induction results (≥20% of the positive control response).

In donor 3, Compound 2 did not increase CYP1A2 mRNA at any of the three tested concentrations. CYP1A2 enzyme activity was not affected at 5 or 20 μM. Compound 2 at 10 μM showed 3.24-fold increase in CYP1A2 enzyme activity, with 11.5% of the response of the positive control. Compound 2 did not increase either mRNA or enzyme activity of CYP2B6 or CYP3A4 at any of the three tested concentrations.

### Example 21: P-GP and BCRP Substrate and Inhibition Assessments Using CACO-2 and/or MDR-1-MDCK Cell Monolayers

The P-GP substrate and inhibition potential of Compound 2 was determined using Caco-2 and MDR1-MDCK cell monolayers. The BCRP substrate and inhibition potential of Compound 2 was also determined using Caco-2 and BCRP-MDCK cell monolayers.

P-GP Substrate Assessment Experimental Procedure: Caco-2 cells (clone C2BBe1) were obtained from American Type Culture Collection. MDR1-MDCK cells were obtained from the National Institutes of Health. Cell monolayers were grown to confluence on collagen-coated, microporous membranes in 12-well assay plates. The permeability assay buffer was Hanks' balanced salt solution (HBSS) containing 10 mM HEPES and 15 mM glucose at a pH of 7.4. The buffer in the receiver chamber also contained 1% bovine serum albumin. The dosing solution concentrations were 0.3 and 5 μM of test article in the assay buffer+/−1 μM valspodar. Cells were first pre-incubated for 30 minutes with HBSS containing+/−1 μM valspodar. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow was also measured post-experimentally for each monolayer to ensure no damage was inflicted to the cell monolayers during the flux period. All samples were assayed by LC-MS/MS using electrospray ionization. The apparent permeability (Papp) and percent recovery were calculated as follows:

Papp=(dCr/dt)×Vr/(A×C_{A}) (1)

Percent Recovery=100×((Vr×Cr^{final})+(V_{d}×C_{d}^{final}))/(V_{d}×C_{N}) (2)

where, dCr/dt is the slope of the cumulative concentration in the receiver compartment versus time in μM s⁻¹; Vr is the volume of the receiver compartment in cm³; Vd is the volume of the donor compartment in cm³; A is the area of the insert (1.13 cm² for 12-well); C_{A} is the average of the nominal dosing concentration and the measured 120 minute donor concentration in μM; CN is the nominal concentration of the dosing solution in μM; Cr^{final} is the cumulative receiver concentration in μM at the end of the incubation period; Cd^{final} is the concentration of the donor in μM at the end of the incubation period. An efflux ratio (ER) was defined as Pₐₚₚ (B-to-A)/Pₐₚₚ (A-to-B).

The cell batch quality control results for Caco-2 are shown in TABLE 128. The cell batch quality control results for MDR1-MDCK are shown in TABLE 128.

TABLE 128

| Plates | 12-well |
|---|---|
| Passage Number | 62 |
| Age at QC (days) | 21 |
| Age at Experiment (days) | 22 |
| Atenolol Pₐₚₚ, 10⁻⁶ cm/s | 0.149 |
| Propranolol Pₐₚₚ, 10⁻⁶ cm/s | 20.5 |
| Digoxin A-to-B Pₐₚₚ, 10⁻⁶ cm/s | 0.403 |
| Digoxin B-to-A Pₐₚₚ, 10⁻⁶ cm/s | 8.93 |
| Digoxin Efflux Ratio | 22.2 |

Acceptance Criteria
≤0.5
10-30
N/A
N/A
≥10

TABLE 129

| Plates | 12-well |
|---|---|
| Passage Number | 22 |
| Age at QC (days) | 7 |
| Age at Experiment (days) | 8 |
| Atenolol Pₐₚₚ, 10⁻⁶ cm/s | 0.0746 |
| Propranolol Pₐₚₚ, 10⁻⁶ cm/s | 20.3 |
| Digoxin A-to-B Pₐₚₚ, 10⁻⁶ cm/s | 0.0792 |
| Digoxin B-to-A Pₐₚₚ, 10⁻⁶ cm/s | 11.0 |
| Digoxin Efflux Ratio | 139 |

Acceptance Criteria
≤0.5
10-30
N/A
N/A
≥10

The experimental results for Caco-2 are shown in TABLE 130. The experimental results for MDR1-MDCK are shown in TABLE 131.

TABLE 130

| | | | | | | | P-gp |
|---|---|---|---|---|---|---|---|
| | | Recovery | Pₐₚₚ (10⁻⁶ cm/s) | | | Efflux | Substrate |
| Test Article | Direction | (%) | R1 | R2 | AVG | Ratio | Classification |
| 0.3 μM Compound 2 | A-to-B | 59.2 | 15.9 | 17.5 | 16.7 | 2.69 | Negative |
| | B-to-A | 60.6 | 44.9 | 44.6 | 44.8 | | |
| 0.3 μM Compound 2 + | A-to-B | 59.3 | 20.2 | 22.2 | 21.2 | 2.10 | |
| 1 μM Valspodar | B-to-A | 71.1 | 47.0 | 41.9 | 44.4 | | |
| 5 μM Compound 2 | A-to-B | 56.4 | 20.1 | 18.3 | 19.2 | 2.06 | Positive |
| | B-to-A | 73.3 | 40.7 | 38.5 | 39.6 | | |
| 5 μM Compound 2 + | A-to-B | 60.6 | 30.1 | 23.3 | 26.7 | 1.21 | |
| 1 μM Valspodar | B-to-A | 88.5 | 36.6 | 28.1 | 32.3 | | |
| Digoxin | A-to-B | 73.4 | 0.344 | 0.645 | 0.494 | 22.1 | Positive |
| | B-to-A | 79.9 | 11.4 | 10.5 | 10.9 | | |
| Digoxin + | A-to-B | 72.2 | 2.39 | 1.73 | 2.06 | 1.20 | |
| 1 μM Valspodar | B-to-A | 95.3 | 2.70 | 2.24 | 2.47 | | |

P-gp Substrate Classification:
CER ≥1.0 without valspodar, and reduced by ≥50% with valspodar: Positive
CER ≥1.0 without valspodar, and reduced by <50% with valspodar: Negative
CER <1.0 without and with valspodar: Negative
CER = Corrected Efflux Ratio = ER − 1

TABLE 131

| | | | | | | | P-gp |
|---|---|---|---|---|---|---|---|
| | | Recovery | Pₐₚₚ (10⁻⁶ cm/s) | | | Efflux | Substrate |
| Test Article | Direction | (%) | R1 | R2 | AVG | Ratio | Classification |
| 0.3 μM Compound 2 | A-to-B | 86.1 | 1.03 | 1.10 | 1.07 | 44.6 | Positive |
| | B-to-A | 86.4 | 53.8 | 41.2 | 47.5 | | |
| 0.3 μM Compound 2 + | A-to-B | 68.6 | 26.3 | 20.5 | 23.4 | 0.626 | |
| 1 μM Valspodar | B-to-A | 69.3 | 16.3 | 13.0 | 14.6 | | |
| 5 μM Compound 2 | A-to-B | 90.8 | 0.750 | 0.665 | 0.708 | 83.0 | Positive |
| | B-to-A | 102 | 59.9 | 57.5 | 58.7 | | |
| 5 μM Compound 2 + | A-to-B | 63.2 | 16.9 | 9.72 | 13.3 | 2.33 | |
| 1 μM Valspodar | B-to-A | 91.4 | 25.6 | 36.5 | 31.1 | | |
| Digoxin | A-to-B | 77.6 | 0.0436 | 0.0205 | 0.0321 | 370 | Positive |
| | B-to-A | 74.9 | 10.4 | 13.3 | 11.9 | | |
| Digoxin + | A-to-B | 81.9 | 0.374 | 0.498 | 0.436 | 1.19 | |
| 1 μM Valspodar | B-to-A | 85.9 | 0.472 | 0.565 | 0.519 | | |

P-gp Substrate Classification:
CER ≥1.0 without valspodar, and reduced by ≥50% with valspodar: Positive
CER ≥1.0 without valspodar, and reduced by <50% with valspodar: Negative
CER <1.0 without and with valspodar: Negative
CER = Corrected Efflux Ratio = ER − 1

P-GP Inhibition Assessment Experimental Procedure: Caco-2 cells (clone C2BBe1) were obtained from American Type Culture Collection. Cell monolayers were grown to confluence on collagen-coated, microporous membranes in 12-well assay plates. The permeability assay buffer was HBSS containing 10 mM HEPES and 15 mM glucose at a pH of 7.4. The dosing solution concentration was 10 μM of digoxin in the assay buffer+/−10 μM or 100 μM Compound 2. Cells were first pre-incubated for 30 minutes with HBSS containing+/−10 or 100 μM Compound 2. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow was also measured post-experimentally for each monolayer to ensure no damage was inflicted to the cell monolayers during the flux period. All samples were assayed by LC-MS/MS using electrospray ionization. The apparent permeability (Papp) and percent recovery were calculated as follows:

Papp=(dCr/dt)×Vr/(A×C_{A}) (1)

Percent Recovery=100×((Vr×Cr^{final})+(V_{d}×C_{d}^{final}))/(V_{d}×C_{N}) (2)

where, dCr/dt is the slope of the cumulative concentration in the receiver compartment versus time in μM s⁻¹; Vr is the volume of the receiver compartment in cm³; V_{d} is the volume of the donor compartment in cm³; A is the area of the insert (1.13 cm² for 12-well); C_{A} is the average of the nominal dosing concentration and the measured 120 minute donor concentration in μM; C_{N} is the nominal concentration of the dosing solution in μM; Cr^{final} is the cumulative receiver concentration in μM at the end of the incubation period; and Cd^{final} is the concentration of the donor in μM at the end of the incubation period. The ER was defined as Pₐₚₚ (B-to-A)/Pₐₚₚ (A-to-B).

Cell batch quality control results of the Caco-2 cells used in the P-GP inhibition assessment are shown in TABLE 128 above. TABLE 132 shows the experimental results of the P-GP inhibition assessment.

TABLE 132

| | | | | | | | P-gp |
|---|---|---|---|---|---|---|---|
| | | Recovery | Pₐₚₚ (10⁻⁶ cm/s) | | | Efflux | Inhibition |
| Test Article | Direction | (%) | R1 | R2 | AVG | Ratio | Classification |
| Digoxin | A-to-B | 73.4 | 0.344 | 0.645 | 0.494 | 22.1 | — |
| | B-to-A | 79.9 | 11.4 | 10.5 | 10.9 | | |
| Digoxin + 10 μM | A-to-B | 75.6 | 2.23 | 2.66 | 2.44 | 1.72 | Positive |
| Compound 2 | B-to-A | 86.1 | 5.11 | 3.31 | 4.21 | | |
| Digoxin + 100 μM | A-to-B | 86.9 | 2.59 | 2.09 | 2.34 | 1.11 | Positive |
| Compound 2* | B-to-A | 97.9 | 2.60 | 2.60 | 2.60 | | |
| Digoxin + 100 μM | A-to-B | 72.2 | 2.39 | 1.73 | 2.06 | 1.20 | Positive |
| Compound 2* | B-to-A | 95.3 | 2.70 | 2.24 | 2.47 | | |

*All monolayers dosed with 100 μM Compound 2 failed the lucifer yellow monolayer integrity test criteria (≤0.8 × 10−6 cm/s). The 100 μM dosing solutions of Compound 2 showed slight precipitation of the test article.
P-gp Inhibition Classification:
Digoxin CER ≥1.0 without inhibitor, and reduced by ≥50% with inhibitor: Positive
Digoxin CER ≥1.0 without inhibitor, and reduced by <50% with inhibitor: Negative
Digoxin CER <1.0 without and with inhibitor: Negative
CER = Corrected Efflux Ratio = Efflux Ratio − 1

PCRP Substrate Assessment Experimental Procedure: Caco-2 cells (clone C2BBe1) were obtained from American Type Culture Collection. BCRP-MDCK cell monolayers were prepared at Absorption Systems. Cell monolayers were grown to confluence on collagen-coated, microporous membranes in 12-well assay plates. The permeability assay buffer was HBSS containing 10 mM HEPES and 15 mM glucose at a pH of 7.4. The buffer in the receiver chamber also contained 1% bovine serum albumin. The dosing solution concentrations were 0.3 and 5 μM of test article in the assay buffer+/−0.5 μM Ko143. Cells were first pre-incubated for 30 minutes with HBSS containing+/−0.5 μM Ko143. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow post-experimentally was also measured for each monolayer to ensure no damage was inflicted to the cell monolayers during the flux period. All samples were assayed by LC-MS/MS using electrospray ionization. The apparent permeability (Papp) and percent recovery were calculated as described in the P-GP substrate assessment experimental procedure described above.

Cell batch quality control results of the Caco-2 cells used in the PCRP inhibition assessment are shown in TABLE 128 above. Cell batch quality control results of the BCRP-MDCK2 cells used in the PCRP inhibition assessment are shown in TABLE 133.

TABLE 133

| Plates | 12-well |
|---|---|
| Passage Number | 23 |
| Age at QC (days) | 7 |
| Age at Experiment (days) | 8 |
| Atenolol Pₐₚₚ, 10⁻⁶ cm/s | 0.0895 |
| Propranolol Pₐₚₚ, 10⁻⁶ cm/s | 20.1 |
| Cladribine A-to-B Pₐₚₚ, 10⁻⁶ cm/s | 0.136 |
| Cladribine B-to-A Pₐₚₚ, 10⁻⁶ cm/s | 18.4 |
| Cladribine Efflux Ratio | 135 |

Acceptance Criteria
≤0.5
10-30
N/A
N/A
≥50

Experimental results of the PCRP substrate assessment of Caco-2 cells are shown in TABLE 134. Experimental results of the PCRP substrate assessment of BCRP-MDCK cell monolayers are shown in TABLE 135.

TABLE 134

| | | | | | | | BCRP |
|---|---|---|---|---|---|---|---|
| | | Recovery | Pₐₚₚ (10⁻⁶ cm/s) | | | Efflux | Substrate |
| Test Article | Direction | (%) | R1 | R2 | AVG | Ratio | Classification |
| 0.3 μM Compound 2 | A-to-B | 59.2 | 15.9 | 17.5 | 16.7 | 2.69 | Negative |
| | B-to-A | 60.6 | 44.9 | 44.6 | 44.8 | | |
| 0.3 μM Compound 2 + | A-to-B | 55.0 | 15.2 | 14.4 | 14.8 | 3.33 | |
| 0.5 μM Ko143 | B-to-A | 64.2 | 48.7 | 49.8 | 49.2 | | |
| 5 μM Compound 2 | A-to-B | 56.4 | 20.1 | 18.3 | 19.2 | 2.06 | Negative |
| | B-to-A | 73.3 | 40.7 | 38.5 | 39.6 | | |
| 5 μM Compound 2 + | A-to-B | 57.5 | 21.0 | 23.6 | 22.3 | 1.95 | |
| 0.5 μM Ko143 | B-to-A | 77.1 | 35.7 | 51.2 | 43.5 | | |
| Cladribine | A-to-B | 80.5 | 0.681 | 0.478 | 0.580 | 32.0 | Positive |
| | B-to-A | 85.6 | 17.7 | 19.3 | 18.5 | | |
| Cladribine + | A-to-B | 89.8 | 0.654 | 1.28 | | 2.44 | |
| 0.5 μM Ko143 | B-to-A | 87.7 | 1.91 | 2.81 | 2.36 | | |

BCRP Substrate Classification:
CER ≥1.0 without Ko143, and reduced by ≥50% with Ko143: Positive
CER ≥1.0 without Ko143, and reduced by <50% with Ko143: Negative
CER <1.0 without and with Ko143: Negative
CER = Corrected Efflux Ratio = ER − 1

TABLE 135

| | | | | | | | BCRP |
|---|---|---|---|---|---|---|---|
| | | Recovery | Pₐₚₚ (10⁻⁶ cm/s) | | | Efflux | Substrate |
| Test Article | Direction | (%) | R1 | R2 | AVG | Ratio | Classification |
| 0.3 μM Compound 2 | A-to-B | 84.8 | 5.41 | 5.69 | 5.55 | 6.77 | Positive |
| | B-to-A | 74.8 | 34.1 | 41.1 | 37.6 | | |
| 0.3 μM Compound 2 + | A-to-B | 75.6 | 10.7 | 10.3 | 10.5 | 2.73 | |
| 0.5 μM Ko143 | B-to-A | 76.6 | 27.3 | 30.0 | 28.6 | | |
| 5 μM Compound 2 | A-to-B | 83.0 | 5.83 | 4.06 | 4.94 | 3.61 | Positive |
| | B-to-A | 82.5 | 14.0 | 21.7 | 17.9 | | |
| 5 μM Compound 2 + | A-to-B | 72.4 | 14.6 | 11.2 | 12.9 | 1.46 | |
| 0.5 μM Ko143 | B-to-A | 80.4 | 17.9 | 19.6 | 18.8 | | |
| Cladribine | A-to-B | 91.3 | 0.142 | 0.280 | 0.211 | 83.3 | Positive |
| | B-to-A | 92.9 | 15.6 | 19.6 | 17.6 | | |
| Cladribine + | A-to-B | 93.5 | 0.354 | 0.514 | 0.434 | 0.644 | |
| 0.5 μM Ko143 | B-to-A | 107 | 0.273 | 0.286 | 0.279 | | |

BCRP Substrate Classification:
CER ≥1.0 without Ko143, and reduced by ≥50% with Ko143: Positive
CER ≥1.0 without Ko143, and reduced by <50% with Ko143: Negative
CER <1.0 without and with Ko143: Negative
CER = Corrected Efflux Ratio = ER − 1

BCRP Inhibition Assay Experimental Procedure: Caco-2 cells (clone C2BBe1) were obtained from American Type Culture Collection. Cell monolayers were grown to confluence on collagen-coated, microporous membranes in 12-well assay plates. The permeability assay buffer was HBSS containing 10 mM HEPES and 15 mM glucose at a pH of 7.4. The dosing solution concentration was 10 μM of cladribine in the assay buffer+/−10 μM or 100 μM Compound 2. Cell monolayers were first pre-incubated for 30 minutes with assay buffer+/−10 μM or 100 μM Compound 2. After 30 minutes the buffer was removed, replaced with fresh dosing solution/assay buffer, and time was recorded as 0. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow post-experimentally was also measured for each monolayer to ensure no damage was inflicted to the cell monolayers during the flux period. All samples were assayed by LC-MS/MS using electrospray ionization. The apparent permeability (Pₐₚₚ) and percent recovery were calculated as described in the P-GP inhibition assessment experimental procedure above. Cell batch quality control results for the Caco-2 cells were as shown in TABLE 128.

Experimental results of the BCRP inhibition assay are shown in TABLE 136.

TABLE 136

| | | Recovery | Pₐₚₚ (10⁻⁶ cm/s) | | | Efflux | BCRP |
|---|---|---|---|---|---|---|---|
| Test Article | Direction | (%) | R1 | R2 | AVG | Ratio | Inhibition |
| Cladribine | A-to-B | 80.5 | 0.681 | 0.478 | 0.580 | 32.0 | — |
| | B-to-A | 85.6 | 17.7 | 19.3 | 18.5 | | |
| Cladribine + 10 μM | A-to-B | 69.0 | 0.475 | 0.471 | 0.473 | 3.12 | Positive |
| Compound 2 | B-to-A | 88.8 | 1.72 | 1.24 | 1.48 | | |
| Cladribine + 100 μM | A-to-B | 68.8 | 1.04 | 0.624 | 0.834 | 1.30 | Positive |
| Compound 2* | B-to-A | 80.5 | 0.840 | 1.33 | 1.09 | | |
| Cladribine + 0.5 μM | A-to-B | 89.8 | 0.654 | 1.28 | 0.967 | 2.44 | Positive |
| Ko143 | B-to-A | 87.7 | 1.91 | 2.81 | 2.36 | | |

*All monolayers dosed with 100 μM Compound 2 failed the lucifer yellow monolayer integrity test criteria (≤0.8 × 10⁻⁶ cm/s). The 100 μM dosing solutions of Compound 2 showed slight precipitation of the test article.
BCRP Inhibition Classification:
Cladribine CER ≥1.0 without inhibitor, and reduced by ≥50% with inhibitor: Positive
Cladribine CER ≥1.0 without inhibitor, and reduced by <50 with inhibitor: Negative
Cladribine CER <1.0 without and with inhibitor: Negative
CER = Corrected Efflux Ratio = ER − 1

Liquid chromatography was performed as described in EXAMPLE 20 with a 1-10 μL injection volume for the autosampler. Mass spectrometry was performed using a PE SCIEX API 4000; Turbo Ionspray interface; Multiple reaction monitoring mode; and 1.0 minute methods. The settings of the mass spectrometry experiments are shown in TABLE 137.

TABLE 137

| Test Article | +/− | Q1 | Q3 | DP | EP | CE | CXP | IS |
|---|---|---|---|---|---|---|---|---|
| Compound 2 | + | 546.3 | 114.2 | 100 | 10 | 35 | 11 | 5500 |

TEM: 500;
CAD: 7;
CUR: 30;
GS1: 50;
GS2: 50

Conclusions: Compound 2 showed a slight degree of P-GP substrate potential in the Caco-2 cell line, but was clearly indicated to be a P-GP substrate in MDR1-MDCK cells. The P-GP inhibition potential of Compound 2 was demonstrated in the Caco-2 cell line at the 10 μM dosing concentration. The BCRP substrate potential assessment of Compound 2 did not show BCRP substrate potential in the Caco-2 cell line, but did show a small degree of BCRP substrate potential in the BCRP-MCDK cell line. The BCRP inhibition potential of Compound 2 was demonstrated in the Caco-2 cell line at the 10 μM dosing concentration. All experiments passed the lucifer yellow monolayer integrity test criteria (≤0.8×10−6 cm/s). TABLE 138 shows a summary of the substrate assessments of Compound 2. TABLE 139 shows a summary of the inhibition assessments of Compound 2.

TABLE 138

| Test | Cell | Dosing | P-gp Substrate | BCRP |
|---|---|---|---|---|
| Article | Line | Concentration | Classification | Substrate |
| Compound 2 | Caco-2 | 0.3 | Negative | Negative |
| | | 5 | Positive | Negative |
| | MDR1- | 0.3 | Positive | — |
| | MDCK | 5 | Positive | — |
| | BCRP- | 0.3 | — | Positive |
| | MDCK | 5 | — | Positive |

TABLE 139

| Test | Dosing | P-gp Inhibition | BCRP |
|---|---|---|---|
| Article | Concentration | Classification | Substrate |
| Compound 2 | 10 | Positive | Positive |
| | 100* | Positive | Positive |

*Experiments dosed at 100 μM failed the lucifer yellow monolayer integrity test criteria (≤0.8 × 10−6 cm/s), possibly due to cellular toxicity of Compound 2.

### Example 22: IC₅₀ Determination for P-GP and BCRP Inhibition Using Caco-2 Cell Monolayers

IC₅₀ values for P-GP and BCRP inhibition by Compound 2 were determined using Caco-2 cell monolayers.

Experimental procedure for determining P-GP IC₅₀ values: Caco-2 cell monolayers were grown to confluence on collagen-coated, microporous membranes in 12-well assay plates. The permeability assay buffer was HBSS containing 10 mM HEPES and 15 mM glucose at a pH of 7.4. A stock solution of digoxin was prepared at 10 mM in DMSO. Compound 2 stock solutions were prepared in DMSO at several concentrations (10 mM, 3.33 mM, 1.11 mM, 0.370 mM, 0.123 mM, 0.0412 mM). Dosing solutions were prepared by direct addition of 4 μL of stock solution of digoxin and 4 μL of stock solution of Compound 2 into 4.0 mL of assay buffer. The final DMSO concentration was 0.2% in all experiments. Cell monolayers were dosed on the basolateral side (B-to-A) and incubated at 37° C. with 5% CO₂ in a humidified incubator. Samples were taken from the receiver and donor chambers at 120 minutes. Each determination was performed in duplicate. The flux of lucifer yellow was also measured post experimentally for each monolayer to ensure no damage was inflicted to the cell monolayers during the flux period. All samples were assayed by LC-MS/MS using electrospray ionization. The apparent permeability, Papp, and percent recovery were calculated as follows:

Papp=(dCr/dt)×Vr/(A×C_{A}) (1)

Percent Recovery=100×((Vr×Cr^{final})+(V_{d}×C_{d}^{final}))/(V_{d}×C_{N}) (2)

where dCᵣ/dt is the slope of the cumulative concentration in the receiver compartment versus time in μM s−1; Vᵣ is the volume of the receiver compartment in cm³; V_{d} is the volume of the donor compartment in cm³; A is the area of the insert (1.13 cm² for 12-well); C₀ is the measured concentration of the donor chamber at time 0 in μM; Cᵣfinal is the cumulative receiver concentration in μM at the end of the incubation period; and C_{d}final is the concentration of the donor in μM at the end of the incubation period. Cell batch quality control results for Caco-2 cells are shown in TABLE 140. The experimental results are shown in TABLE 141.

TABLE 140

| Plates: | 12-well |
|---|---|
| Passage: | 66 |
| Age at QC (days) | 20 |
| Age at Experiment (days) | 27 |
| Atenolol Pₐₚₚ, 10⁻⁶ cm/s | 0.169 |
| Propranolol Pₐₚₚ, 10⁻⁶ cm/s | 20.7 |
| Digoxin A-to-B Pₐₚₚ, 10⁻⁶ cm/s | 0.511 |
| Digoxin B-to-A Pₐₚₚ, 10⁻⁶ cm/s | 9.05 |
| Digoxin Efflux Ratio | 17.7 |

Acceptance Criteria
≤0.5
10-30
N/A
N/A
≥10

TABLE 141

| | Inhibitor | | % | Pₐₚₚ (10⁻⁶ cm/s) | | | % | IC₅₀ |
|---|---|---|---|---|---|---|---|---|
| Test Article | Conc (μM) | Direction | Recovery | R1 | R2 | AVG | Inhibition^{a} | (μM) |
| Digoxin | — | B-A | 95.4 | 13.4 | 12.0 | 12.7 | — | — |
| Digoxin + | 1 | B-A | 85.5 | 3.73 | 3.96 | 3.84 | 100 | — |
| Valspodar | | | | | | | | |
| Digoxin + | 10 | B-A | 90.7 | 6.64 | 5.58 | 6.11 | 74.4 | 7.26 |
| Compound 2 | 3.33 | B-A | 93.8 | 7.87 | 16.4 | 12.1 | 6.25 | |
| | 1.11 | B-A | 84.9 | 10.6 | 12.8 | 11.7 | 10.8 | |
| | 0.370 | B-A | 80.9 | 13.4 | 12.2 | 12.8 | 0 | |
| | 0.123 | B-A | 87.4 | 8.79 | 14.7 | 11.7 | 10.8 | |
| | 0.0412 | B-A | 90.1 | 13.3 | 12.5 | 12.9 | 0 | |

^{a}Percent inhibition values calculated as negative are reported as 0.

BCRP IC₅₀ Determination, Experimental Procedure: Caco-2 cell monolayers were grown to confluence on collagen-coated, microporous membranes in 12-well assay plates. The permeability assay buffer was Hanks Balanced Salt Solution containing 10 mM HEPES and 15 mM glucose at a pH of 7.4. A stock solution of cladribine was prepared at 10 mM in DMSO. Test article stock solutions were prepared in DMSO at several concentrations (10 mM, 3.33 mM, 1.11 mM, 0.370 mM, 0.123 mM, 0.0412 mM). Dosing solutions were prepared by direct addition of 4 μL of stock solution of cladribine and 4 μL of stock solution of test article into 4.0 mL of assay buffer. The final DMSO concentration was 0.2% in all experiments. Cell monolayers were dosed on the basolateral side (B-to-A) and incubated at 37° C. with 5% CO2 in a humidified incubator. Samples were taken from the receiver and donor chambers at 120 minutes. Each determination was performed in duplicate. The flux of lucifer yellow was also measured post-experimentally for each monolayer to ensure no damage was inflicted to the cell monolayers during the flux period. All samples were assayed by LC-MS/MS using electrospray ionization. The apparent permeability, Papp, and percent recovery were calculated as described in the P-GP IC₅₀ assay above.

Cell batch quality control results are presented in TABLE 140 above. All experiments passed the lucifer yellow monolayer integrity test (Pₐₚₚ≤0.8×10⁻⁶ cm/s). Experimental results are shown in TABLE 142. A summary of the results are shown in TABLE 143.

TABLE 142

| | Inhibitor | | % | Pₐₚₚ (10⁻⁶ cm/s) | | | % | IC₅₀ |
|---|---|---|---|---|---|---|---|---|
| Test Article | Conc (μM) | Direction | Recovery | R1 | R2 | AVG | Inhibition^{a} | (μM) |
| Cladribine | — | B-A | 103 | 17.8 | 11.5 | 14.6 | — | — |
| Cladribine + | 0.5 | B-A | 85.3 | 2.42 | 1.97 | 2.19 | 100 | — |
| Ko143 | | | | | | | | |
| Cladibrine + | 10 | B-A | 102 | 1.43 | 1.49 | 1.46 | 106 | 1.35 |
| Compound 2 | 3.33 | B-A | 93.8 | 2.65 | 4.42 | 3.54 | 89.2 | |
| | 1.11 | B-A | 90.7 | 10.5 | 7.31 | 8.91 | 46.0 | |
| | 0.370 | B-A | 93.8 | 14.4 | 13.3 | 13.8 | 6.42 | |
| | 0.123 | B-A | 117 | 16.3 | 15.7 | 16.0 | 0 | |
| | 0.0412 | B-A | 98.0 | 14.5 | 12.2 | 13.4 | 10.1 | |

^{a}Percent inhibition values calculated as negative are reported as 0.

TABLE 143

| | IC₅₀ for P-GP | IC₅₀ for BCRP |
|---|---|---|
| Test Article | Inhibition (μM) | Inhibition (μM) |
| Compound 2 | 7.26 | 1.35 |

### Example 23: Inhibition of Cytochrome P450 Enzymes in Human Liver Microsomes by Compound 2

The IC₅₀ values of Compound 2 in the inhibition of CYP1A2, CYP2B6, CYP2C8, CYP2C9, CYP2C19, CYP2D6, and CYP3A were determined in human liver microsomes (HLM).

Materials and reagents: A stock solution of Compound 2 (100 mM) was prepared in dimethyl sulfoxide (DMSO) and diluted using methanol. CYP probe substrates and metabolites, and positive inhibitors are shown in TABLE 144 and TABLE 145, respectively. All other chemicals and reagents were of analytical grade or higher. Pooled human liver microsomes (HLMs) were obtained from 200 donors of mixed gender and races. The HLMs were stored at −80° C. until use.

TABLE 144

| CYP | Probe Substrate | Metabolite |
|---|---|---|
| CYP1A2 | Phenacetin (63 μM) | Acetaminophen |
| CYP2B6 | Bupropion (75 μM) | OH bupropion |
| CYP2C8 | Amodiaquine (2 μM) | Desethylamodiaquine |
| CYP2C9 | Diclofenac (10 μM) | 4′-OH diclofenac |
| CYP2C19 | S-mephenytoin(40 μM) | 4′-OH mephenytoin |
| CYP2D6 | Bufuralol (7 μM) | 1′-OH bufuralol |
| CYP3A | Midazolam (2.5 μM) | 1′-OH midazolam |
| | Testosterone (55 μM) | 6β-OH testosterone |

TABLE 145

| CYP | Inhibitor |
|---|---|
| CYP1A2 | α-Naphthoflavone |
| CYP2B6 | Thio-TEPA (N, N′, N″-triethylenethiophosphoramide |
| CYP2C8 | Montelukast |
| CYP2C9 | Sulfaphenazole |
| CYP2C19 | (+)-N-3-benzylnirvanol |
| CYP2D6 | Quinidine |
| CYP3A | Ketoconazole |

Compound 2, at eight concentrations (0-100 μM), was incubated with pooled HLM (0.25 mg protein/mL) in phosphate buffer (100 mM, pH 7.4) containing MgCl₂ (5 mM), NADPH (1 mM), and an individual CYP probe substrate (˜Kₘ). The total organic solvent content in the final incubation was less than 1% (DMSO≤0.1%, other organic solvent≤1%). The reaction mixture without NADPH was equilibrated in a shaking water bath at 37° C. for 5 minutes. The reaction was initiated by the addition of NADPH (1 mM), followed by incubation at 37° C. for 10-30 minutes depending on the individual CYP isoform. CYP3A was incubated at 37° C. for 10 min; CYP2C19 was incubated at 37° C. for 30 min; and all other CYPs were incubated at 37° C. for 20 min. The reaction was terminated by adding two volumes of ice-cold acetonitrile containing an internal standard (IS, stable isotope-labeled CYP probe metabolite) (ACN containing IS 1:2, v/v). Negative (vehicle) controls were conducted without a test article. Positive controls were performed in parallel using known CYP inhibitors. After the removal of protein by centrifugation at 1,640 g for 10 minutes at 4° C., the supernatants were transferred to an HPLC autosampler plate. The formation of individual CYP probe metabolites was determined by LC-MS/MS.

Liquid Chromatography was performed using a Thermo Hypersil BDS C₁₈ 30×2.1 mm i.d., 3 μm, column with guard cartridge; 25 mM ammonium formate, pH 3.5 buffer; 90% water/10% buffer mobile phase A; 90% ACN/10% buffer mobile phase B; and a flow rate of 0.3-0.35 mL/minute. The run time was 2.5-6 minutes; injection volume was 10-30 μL; and autosampler wash was water/methanol/2-propanol: 1/1/1 (v/v/v) with 0.2% formic acid. Determination of Acetaminophen (CYP1A2) was performed using the gradient program shown in TABLE 146. Determination of 4′-OH Diclofenac (CYP2C9) was performed using the gradient program shown in TABLE 147. Determination of 4′-OH Mephenytoin (CYP2C19) was performed using the gradient program shown in TABLE 148. Determination of 6β-OH Testosterone (CYP3A) was performed using the gradient program shown in TABLE 149. Determination of CYP Probe Metabolites (all other CYPs) was performed using the gradient program shown in TABLE 150.

TABLE 147

| Time (min) | % A | % B |
|---|---|---|
| 0.0 | 100 | 0 |
| 1.2 | 60 | 40 |
| 3.0 | 0 | 100 |
| 3.1 | 100 | 0 |
| 4.5 | 100 | 0 |

TABLE 146

| Time (min) | % A | % B |
|---|---|---|
| 0.0 | 100 | 0 |
| 0.6 | 70 | 30 |
| 2.0 | 0 | 100 |
| 2.1 | 100 | 0 |
| 3.0 | 100 | 0 |

TABLE 148

| Time (min) | % A | % B |
|---|---|---|
| 0.0 | 100 | 0 |
| 0.9 | 70 | 30 |
| 3.0 | 0 | 100 |
| 3.1 | 100 | 0 |
| 4.0 | 100 | 0 |
| 4.5 | 100 | 0 |

TABLE 149

| Time (min) | % A | % B |
|---|---|---|
| 0.0 | 80 | 20 |
| 1.0 | 75 | 25 |
| 4.0 | 60 | 40 |
| 4.1 | 0 | 100 |
| 4.5 | 0 | 100 |
| 4.6 | 80 | 20 |
| 6.0 | 80 | 20 |

TABLE 150

| Time (min) | % A | % B |
|---|---|---|
| 0.0 | 100 | 0 |
| 0.8 | 20 | 80 |
| 1.0 | 0 | 100 |
| 1.5 | 0 | 100 |
| 1.6 | 100 | 0 |
| 2.5 | 100 | 0 |

Mass spectrometry was performed using a PE SCIEX API 4000 instrument; Turbo Ionspray interface; and MRM quantification. The MS parameter settings are shown in TABLE 151. Abbreviations—DP: Declustering Potential; EP: Entrance Potential; CE: Collision Energy; CXP: Collision Cell Exit Potential; ISV: Ion Spray Voltage; TEM: Ion Source Temperature; CAD: Collisionally Activated Dissociation; CUR: Curtain Gas.

TABLE 151

| Analyte | Q1/Q3 | DP | EP | CE | CXP | ISV | TEM | CAD | CUR | GS1 | GS2 |
|---|---|---|---|---|---|---|---|---|---|---|---|
| Acetaminophen | +152/110 | 80 | 10 | 25 | 21 | 5500 | 500 | 7 | 30 | 50 | 50 |
| Acetaminophen-²H₄ (IS) | +156/114 | 80 | 10 | 25 | 21 | 5500 | 500 | 7 | 30 | 50 | 50 |
| OH Buproprion | +256/139 | 81 | 10 | 37 | 7 | 5500 | 500 | 7 | 30 | 50 | 50 |
| OH Buproprion-²H₆ (IS) | +262/139 | 81 | 10 | 37 | 7 | 5500 | 500 | 7 | 30 | 50 | 50 |
| Desethylamodiaquine | +328/283 | 75 | 10 | 25 | 19 | 5500 | 500 | 7 | 30 | 50 | 50 |
| Desethylamodiaquine-²H₅ (IS) | +333/283 | 75 | 10 | 25 | 19 | 5500 | 500 | 7 | 30 | 50 | 50 |
| 4′-OH Diclofenac | −312/268 | −67 | −10 | −18 | −7 | −4200 | 500 | 7 | 30 | 50 | 50 |
| 4′-OH Diclofenac-²H₄ (IS) | −316/272 | −67 | −10 | −18 | −7 | −4200 | 500 | 7 | 30 | 50 | 50 |
| 4′-OH Mephenytoin | −233/190 | −85 | −10 | −21 | −4 | −4200 | 500 | 7 | 30 | 50 | 50 |
| 4′-OH Mephenytoin-²H₃ (IS) | −236/193 | −85 | −10 | −21 | −4 | −4200 | 500 | 7 | 30 | 50 | 50 |
| 1′-OH Bufuralol | +278/186 | 80 | 10 | 27 | 11 | 5500 | 500 | 7 | 30 | 50 | 50 |
| 1′-OH Bufuralol-²H₉ (IS) | +287/187 | 80 | 10 | 27 | 11 | 5500 | 500 | 7 | 30 | 50 | 50 |
| 1′-OH Midazolam | +342/203 | 106 | 10 | 37 | 12 | 5500 | 500 | 7 | 30 | 50 | 50 |
| 1′-OH Midazolam-¹³C₃ (IS) | +345/206 | 106 | 10 | 37 | 12 | 5500 | 500 | 7 | 30 | 50 | 50 |
| 6ß-OH Testosterone | +305/269 | 80 | 10 | 21 | 18 | 5500 | 500 | 7 | 30 | 50 | 50 |
| 6ß-OH Testosterone-²H₇ (IS) | +312/276 | 80 | 10 | 21 | 18 | 5500 | 500 | 7 | 30 | 50 | 50 |

The percent of control enzyme activity was calculated using the following equation: % of control enzyme activity=100×(enzyme activity in the presence of TA/enzyme activity in the absence of TA). The enzyme activity was expressed as the peak area ratio of probe metabolite to IS, measured by LC-MS/MS. The IC₅₀ values were estimated by fitting the experimental data (percent enzyme activity of control vs. log [inhibitor concentration]) to a sigmoidal model, followed by non-linear regression analysis.

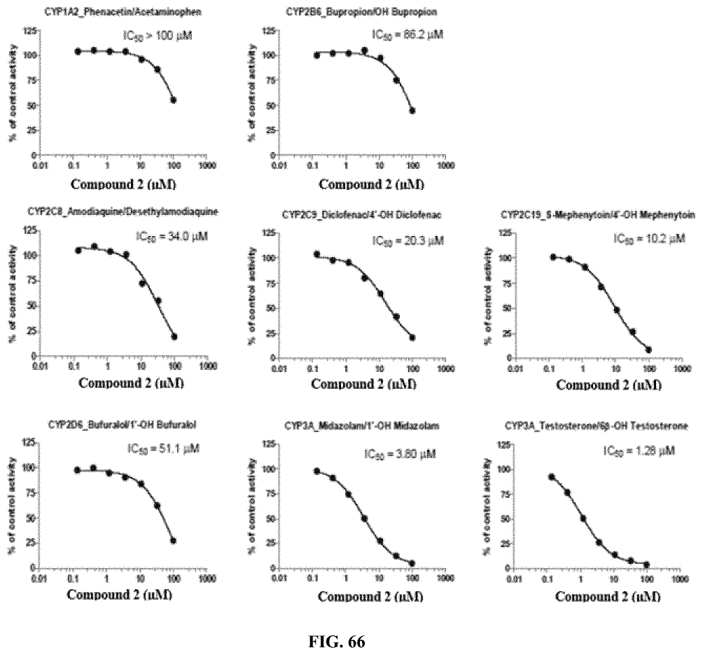

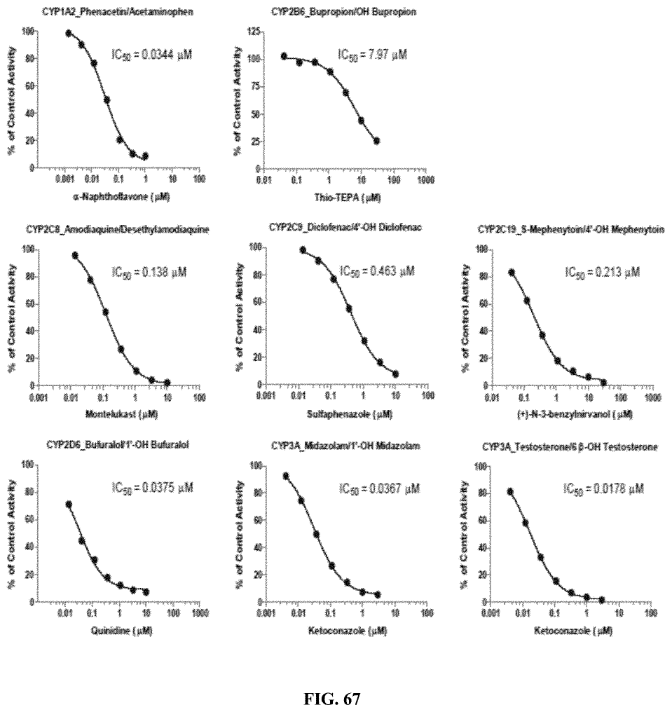

The IC₅₀ values of Compound 2 for the inhibition of CYP activities in HLM are summarized in TABLE 152. The enzyme activity vs. Compound 2 concentration curves are shown in FIG. 66. The inhibition of CYP activities by positive inhibitors is summarized in TABLE 153. The enzyme activity vs. positive inhibitor concentration curves are shown m FIG. 67.

TABLE 152

| | % of Control Enzyme Activity (n = 3)^{a} | | | | | | | | | |
|---|---|---|---|---|---|---|---|---|---|---|
| | Compound | | | | | | | | | IC₅₀ |
| CYP | [symbol] | 0 | 0.137 | 0.412 | 1.23 | 3.70 | 11.1 | 33.3 | 100 | (μM)^{b} |
| CYP1A2 | Average | 100 | 104 | 105 | 104 | 104 | 95.9 | 85.8 | 55.4 | >100 |
| | SD | 3.33 | 5.78 | 5.26 | 2.36 | 5.91 | 2.02 | 3.78 | 4.33 | |
| CYP2B6 | Average | 100 | 99.8 | 102 | 102 | 105 | 97.3 | 75.2 | 44.8 | 86.2 |
| | SD | 3.35 | 0.739 | 5.09 | 3.68 | 2.13 | 3.92 | 3.19 | 2.61 | |
| CYP2C8 | Average | 100 | 105 | 109 | 104 | 101 | 72.6 | 55.4 | 19.6 | 34.0 |
| | SD | 5.15 | 9.53 | 0.985 | 7.89 | 1.68 | 23.1 | 0.894 | 0.274 | |
| CYP2C9 | Average | 100 | 104 | 97.8 | 95.5 | 80.4 | 64.7 | 41.7 | 20.7 | 20.3 |
| | SD | 2.58 | 5.27 | 2.90 | 4.96 | 3.46 | 3.57 | 2.01 | 0.708 | |
| CYP2C19 | Average | 100 | 101 | 98.8 | 91.2 | 71.5 | 48.3 | 26.5 | 8.59 | 10.2 |
| | SD | 7.42 | 10.5 | 4.86 | 2.70 | 6.43 | 4.38 | 1.94 | 0.882 | |
| CYP2D6 | Average | 100 | 98.0 | 100 | 95.1 | 90.8 | 84.2 | 62.5 | 27.7 | 51.1 |
| | SD | 4.91 | 3.84 | 0.971 | 2.05 | 5.67 | 5.11 | 3.56 | 4.37 | |
| CYP3A | Average | 100 | 97.8 | 90.9 | 74.4 | 50.4 | 27.5 | 12.4 | 4.80 | 3.80 |
| (Midazolam) | SD | 4.12 | 2.92 | 3.14 | 1.70 | 0.407 | 1.20 | 0.246 | 0.237 | |
| CYP3A | Average | 100 | 92.4 | 76.8 | 50.8 | 26.1 | 13.8 | 7.61 | 3.65 | 1.28 |
| (Testosterone) | SD | 2.35 | 2.97 | 2.73 | 0.721 | 2.23 | 0.272 | 0.572 | 0.402 | |

^{a}Percent of control enzyme activity = 100 × (Enzyme activity in the presence of TA/Enzyme activity in the absence of TA). Enzyme activity was calculated from the peak area ratio of CYP probe metabolite to IS by LC-MS/MS.
^{b}When the enzyme activity was >50% of control at the highest TA concentration, the ICso is expressed as >the highest concentration. [symbol]
indicates data missing or illegible when filed

TABLE 153

| | | | | | | | | | | IC₅₀ |
|---|---|---|---|---|---|---|---|---|---|---|
| CYP | % of Control Enzyme Activity (n = 3)^{a} | | | | | | | | | (μM)^{b} |
| CYP1A2 | α- | 0 | 0.00137 | 0.00412 | 0.0123 | 0.0370 | 0.111 | 0.333 | 1.00 | 0.0344 |
| | Naphthoflavone | | | | | | | | | |
| | (μM) | | | | | | | | | |
| | Average | 100 | 98.5 | 90.2 | 76.5 | 49.7 | 20.4 | 10.2 | 8.59 | |
| | SD | 1.89 | 4.64 | 1.67 | 4.33 | 3.46 | 3.10 | 0.346 | 0.848 | |
| CYP2B6 | Thio-TEPA | 0 | 0.0412 | 0.123 | 0.370 | 1.11 | 3.33 | 10.0 | 30.0 | 7.97 |
| | (μM) | | | | | | | | | |
| | Average | 100 | 103 | 97.0 | 97.3 | 88.6 | 69.7 | 44.3 | 25.6 | |
| | SD | 1.06 | 5.80 | 11.6 | 15.2 | 5.33 | 6.09 | 1.98 | 2.44 | |
| CYP2C8 | Montelukast | 0 | 0.0137 | 0.0412 | 0.123 | 0.370 | 1.11 | 3.33 | 10.0 | 0.138 |
| | (μM) | | | | | | | | | |
| | Average | 100 | 95.6 | 77.5 | 54.0 | 26.8 | 10.3 | 4.22 | 2.17 | |
| | SD | 3.25 | 4.46 | 2.57 | 2.17 | 0.498 | 0.542 | 0.106 | 0.225 | |
| CYP2C9 | Sulfaphenazole | 0 | 0.0137 | 0.0412 | 0.123 | 0.370 | 1.11 | 3.33 | 10.0 | 0.463 |
| | (μM) | | | | | | | | | |
| | Average | 100 | 98.2 | 90.3 | 76.9 | 55.4 | 31.9 | 16.3 | 7.69 | |
| | SD | 4.80 | 0.545 | 0.162 | 2.16 | 3.26 | 1.61 | 1.38 | 0.779 | |
| CYP2C19 | (+)-N-3- | 0 | 0.0412 | 0.123 | 0.370 | 1.11 | 3.33 | 10.0 | 30.0 | 0.213 |
| | Benzylnirvanol | | | | | | | | | |
| | (μM) | | | | | | | | | |
| | Average | 100 | 83.1 | 62.5 | 37.1 | 18.4 | 10.7 | 6.56 | 2.32 | |
| | SD | 2.95 | 3.76 | 4.40 | 1.23 | 2.64 | 0.259 | 0.270 | 0.639 | |
| CYP2D6 | Quinidine | 0 | 0.0137 | 0.0412 | 0.123 | 0.370 | 1.11 | 3.33 | 10.0 | 0.0375 |
| | (μM) | | | | | | | | | |
| | Average | 100 | 71.1 | 44.7 | 30.6 | 17.7 | 12.1 | 8.69 | 7.28 | |
| | SD | 4.60 | 0.107 | 0.511 | 2.79 | 0.543 | 1.33 | 0.724 | 1.14 | |
| CYP3A | Ketoconazole | 0 | 0.00412 | 0.0123 | 0.0370 | 0.111 | 0.333 | 1.00 | 3.00 | 0.0367 |
| (Midazolam) | (μM) | | | | | | | | | |
| | Average | 100 | 92.5 | 74.5 | 49.9 | 26.6 | 14.4 | 7.30 | 5.38 | |
| | SD | 3.45 | 1.74 | 4.65 | 1.46 | 1.19 | 0.778 | 1.01 | 4.25 | |
| CYP3A | Ketoconazole | 0 | 0.00412 | 0.0123 | 0.0370 | 0.111 | 0.333 | 1.00 | 3.00 | 0.0178 |
| (Testosterone) | (μM) | | | | | | | | | |
| | Average | 100 | 81.4 | 58.4 | 32.9 | 15.4 | 6.75 | 3.54 | 1.56 | |
| | SD | 4.33 | 3.46 | 2.05 | 2.50 | 1.55 | 1.34 | 0.0481 | 0.257 | |

^{a}Percent of control enzyme activity = 100 × (Enzyme activity in the presence of inhibitor/Enzyme activity in the absence of inhibitor). Enzyme activity was calculated from the peak area ratio of CYP probe metabolite to IS by LC-MS/MS.
SD: standard deviation.

Conclusions: The IC₅₀ values of Compound 2 were greater than 100 μM for CYP1A2, 86.2 μM for CYP2B6, 34.0 μM for CYP2C8, 20.3 μM for CYP2C9, 10.2 μM for CYP2C19, 51.1 μM for CYP2D6, 3.80 μM for CYP3A with midazolam as the probe substrate, and 1.28 μM for CYP3A with testosterone as the probe substrate.

### Example 24: Evaluation of Time-Dependent Inhibition of Cytochrome P450 Enzymes in Human Liver Microsomes by Compound 2

The potential for time-dependent inhibition (TDI) of CYP activities (CYP1A2, CYP2B6, CYP2C8, CYP2C9, CYP2C19, CYP2D6, and CYP3A) were determined in HLM by Compound 2.

Materials and Reagents: Compound 2 was prepared as a 100 mM stock solution in DMSO and diluted using methanol. CYP probe substrates and metabolites are shown in TABLE 144 above. CYP positive time-dependent inhibitors are shown in TABLE 154. All other chemicals and reagents were of analytical grade or higher. Pooled human liver microsomes (HLMs) were obtained from 200 donors of mixed gender and races. The HLMs were stored at −80° C. until use.

TABLE 154

| CYP | Inhibitor |
|---|---|
| CYP1A2 | Furafylline |
| CYP2B6 | Thio-TEPA (N,N′,N″-triethylenethiophosphoramide |
| CYP2C8 | Gemfibrozil glucuronide |
| CYP2C9 | Tienilic acid |
| CYP2C19 | Ticlopidine |
| CYP2D6 | Paroxetine |
| CYP3A | Troleandomycin |

CYP TDI was evaluated by a 30-minute pre-incubation of the TA with HLM in the absence and presence of NADPH followed by the CYP enzyme activity assay. The CYP reaction was performed in an incubation volume of 200 μL. Compound 2, at eight concentrations (0-100 μM), was pre-incubated at 37° C. for 30 minutes with HLM (0.25 mg protein/mL) in phosphate buffer (100 mM, pH 7.4) containing MgCl₂ (5 mM) in the absence (reversible incubation conditions) and presence (irreversible incubation conditions) of NADPH (1 mM). The total organic solvent content in the final incubation was less than 1% (DMSO≤0.1%, other organic solvent≤1%). The CYP reaction was initiated by adding an individual CYP probe substrate (˜Kₘ) with (when NADPH was not added in the preincubation step) or without (when NADPH was added in the pre-incubation step) the addition of NADPH (1 mM). The reaction mixture was incubated at 37° C. for 10-30 minutes depending on the individual CYP isoforms. CYP3A samples were incubated for 10 min; CYP2C19 samples were incubated for 30 min; and all other CYPs were incubated for 20 min. The reaction was terminated with ice-cold acetonitrile containing an internal standard (IS, stable isotope-labeled CYP probe metabolite) (CAN:IS 1:2 v/v). Negative (vehicle) controls were conducted using the incubation mixture without the TA. Positive controls were performed in parallel using known CYP time-dependent inhibitors. After the removal of protein by centrifugation at 1,640 g (3,000 rpm) for 10 minutes at 4° C., the supernatants were transferred to an HPLC autosampler plate. The formation of individual CYP probe metabolites was determined by LC-MS/MS. Liquid chromatography and mass spectrometry were performed using parameters and gradient programs described in EXAMPLE 23. Data were analyzed using methods and equations described in EXAMPLE 23. The IC₅₀ shift between reversible incubation conditions (30 minutes of pre-incubation without NADPH) and irreversible incubation conditions (30 minutes of pre-incubation with NADPH) is an index of TDI potential; the threshold for a positive result is IC50 shift>1.5. The IC₅₀ shift and potential for CYP TDI in HLM by Compound 2 are summarized in TABLE 155. The inhibition of CYP activities by positive time-dependent inhibitors is summarized in TABLE 156.

TABLE 155

| | Pre- | | % of Control Enzyme Activity (n = 3) | | | | | | | | | | |
|---|---|---|---|---|---|---|---|---|---|---|---|---|---|
| | incubation | | 0 | 0.137 | 0.412 | 1.23 | 3.70 | 11.1 | 33.3 | 100 | IC₅₀ | IC₅₀ | |
| CYP | (30 min) | | μM | μM | μM | μM | μM | μM | μM | μM | (μM)^{a} | Shift^{b} | TDI |
| CYP1A2 | −NADPH | Average | 100 | 99.4 | 102 | 97.8 | 104 | 98.4 | 92.3 | 58.4 | >100 | ND | Unlikely |
| | | SD | 5.87 | 2.36 | 8.65 | 3.65 | 3.31 | 9.08 | 6.19 | 0.268 | | | |
| | +NADPH | Average | 100 | 103 | 112 | 105 | 108 | 102 | 86.0 | 53.3 | >100 | | |
| | | SD | 13.3 | 10.4 | 3.13 | 8.91 | 13.1 | 7.07 | 7.63 | 3.92 | | | |
| CYP2B6 | −NADPH | Average | 100 | 99.1 | 107 | 103 | 100 | 97.0 | 84.0 | 49.7 | 99.5 | <0.995 | Unlikely |
| | | SD | 3.51 | 1.56 | 2.15 | 1.79 | 0.382 | 7.11 | 3.76 | 4.19 | | | |
| | +NADPH | Average | 100 | 110 | 113 | 116 | 111 | 115 | 93.5 | 56.7 | >100 | | |
| | | SD | 2.45 | 5.23 | 3.99 | 1.41 | 10.3 | 3.73 | 6.37 | 4.63 | | | |
| CYP2C8 | −NADPH | Average | 100 | 98.7 | 97.8 | 96.1 | 84.5 | 90.2 | 64.8 | 26.2 | 55.8 | 1.08 | Unlikely |
| | | SD | 11.6 | 3.82 | 9.44 | 12.1 | 4.83 | 2.24 | 2.76 | 2.46 | | | |
| | +NADPH | Average | 100 | 92.7 | 95.0 | 115 | 98.1 | 89.1 | 58.9 | 29.9 | 51.7 | | |
| | | SD | 15.4 | 7.30 | 3.80 | 10.2 | 0.938 | 7.15 | 11.1 | 5.37 | | | |
| CYP2C9 | −NADPH | Average | 100 | 98.3 | 94.5 | 87.7 | 85.4 | 79.9 | 56.7 | 34.6 | 47.1 | 1.91 | Unlikely |
| | | SD | 0.868 | 7.63 | 2.05 | 6.09 | 5.60 | 5.14 | 3.51 | 1.31 | | | |
| | +NADPH | Average | 100 | 101 | 97.7 | 93.9 | 86.8 | 66.3 | 44.1 | 26.5 | 24.6 | | |
| | | SD | 3.00 | 1.62 | 6.33 | 4.28 | 1.06 | 3.05 | 1.43 | 0.869 | | | |
| CYP2C19 | −NADPH | Average | 100 | 94.1 | 93.3 | 88.9 | 85.1 | 62.7 | 35.4 | 18.8 | 19.5 | 1.55 | Likely |
| | | SD | 6.50 | 1.84 | 6.32 | 2.54 | 4.32 | 2.97 | 7.15 | 3.92 | | | |
| | +NADPH | Average | 100 | 98.9 | 94.3 | 86.2 | 77.3 | 51.2 | 31.2 | 11.3 | 12.6 | | |
| | | SD | 1.19 | 3.07 | 3.60 | 5.88 | 2.46 | 6.41 | 2.94 | 1.54 | | | |
| CYP2D6 | −NADPH | Average | 100 | 103 | 99.5 | 1.2 | 96.7 | 92.9 | 81.5 | 41.1 | 84.8 | 1.23 | Unlikely |
| | | SD | 3.61 | 3.99 | 6.00 | 5.61 | 1.26 | 3.37 | 2.03 | 0.915 | | | |
| | +NADPH | Average | 100 | 97.7 | 101 | 97.7 | 95.7 | 89.2 | 70.4 | 37.0 | 69.2 | | |
| | | SD | 3.33 | NA | 4.29 | 3.97 | 7.02 | 2.86 | 1.37 | 1.88 | | | |
| CYP2A | −NADPH | Average | 100 | 101 | 92.8 | 78.2 | 54.2 | 31.2 | 13.1 | 5.26 | 4.48 | 4.27 | Likely |
| (Midazo- | | SD | 0.829 | 4.84 | 4.32 | 3.51 | 0.984 | 2.77 | 1.25 | 0.968 | | | |
| lam) | +NADPH | Average | 100 | 91.7 | 73.7 | 46.6 | 20.1 | 7.42 | 2.82 | 1.40 | 1.05 | | |
| | | SD | 6.23 | 1.34 | 2.72 | 1.20 | 2.93 | 0.640 | 0.478 | 0.0570 | | | |
| CYP3A | −NADPH | Average | 100 | 95.6 | 83.6 | 54.5 | 29.6 | 16.5 | 7.91 | 4.02 | 1.56 | 2.89 | Likely |
| (Testos- | | SD | 10.0 | 5.10 | 4.66 | 1.90 | 0.241 | 1.26 | 0.451 | 0.205 | | | |
| terone) | +NADPH | Average | 100 | 83.8 | 60.1 | 27.7 | 9.13 | 4.63 | 1.86 | 1.39 | 0.539 | | |
| | | SD | 9.70 | 1.55 | 4.09 | 2.23 | 0.995 | 0.238 | 0.113 | 0.138 | | | |

TABLE 156

| | Pre- | | | | | | | | | | | |
|---|---|---|---|---|---|---|---|---|---|---|---|---|
| | incubation | | | | | | | | | | IC₅₀ | IC₅₀ |
| CYP | (30 min) | | % of Control Enzyme Activity (n = 3) | | | | | | | | (μM)^{a} | Shift^{b} |
| CYP1A2 | Furafylline | — | 0 μM | 0.0412 μM | 0.123 μM | 0.370 μM | 1.11 μM | 3.33 μM | 10.0 μM | 30.0 μM | — | — |
| | −NADPH | Average | 100 | 103 | 99.1 | 84.9 | 70.6 | 39.4 | 16.2 | 8.39 | 2.23 | 7.95 |
| | | SD | 5.63 | 4.93 | 3.43 | 6.70 | 4.21 | 2.48 | 1.23 | 0.624 | | |
| | +NADPH | Average | 100 | 89.6 | 70.2 | 45.2 | 17.7 | 6.05 | 4.28 | 4.04 | 0.280 | |
| | | SD | 3.91 | 7.11 | 4.82 | 2.13 | 0.668 | 0.919 | 0.134 | 0.690 | | |
| CYP2B6 | Thio-TEPA | — | 0 μM | 0.0412 μM | 0.123 μM | 0.370 μM | 1.11 μM | 3.33 μM | 10.0 μM | 30.0 μM | — | — |
| | −NADPH | Average | 100 | 94.5 | 101 | 92.5 | 89.7 | 78.5 | 58.6 | 41.4 | 16.8 | 8.44 |
| | | SD | 8.51 | 7.38 | 10.3 | 5.48 | 8.69 | 4.69 | 2.37 | 1.70 | | |
| | +NADPH | Average | 100 | 98.1 | 93.7 | 86.4 | 63.6 | 37.6 | 18.0 | 11.7 | 1.98 | |
| | | SD | 1.45 | 5.45 | 1.31 | 1.76 | 6.86 | 1.36 | 2.47 | 0.544 | | |
| CYP2C8 | Gemfibrozil | — | 0 μM | 0.0549 μM | 0.165 μM | 0.494 μM | 1.48 μM | 4.44 μM | 13.3 μM | 40.0 μM | — | — |
| | glucuronide | | | | | | | | | | | |
| | −NADPH | Average | 100 | 104 | 107 | 106 | 95.1 | 96.3 | 73.8 | 52.5 | 40.0 | >8.58 |
| | | SD | 4.91 | 2.03 | 11.6 | 2.36 | 3.53 | 6.19 | 7.05 | 0.857 | | |
| | +NADPH | Average | 100 | 103 | 99.1 | 91.7 | 80.1 | 50.6 | 23.4 | 10.1 | 4.66 | |
| | | SD | 7.34 | 11.3 | 2.55 | 2.55 | 3.03 | 2.09 | 5.21 | 1.41 | | |
| CYP2C9 | Tienilic | — | 0 μM | 0.412 μM | 0.123 μM | 0.370 μM | 1.11 μM | 3.33 μM | 10.0 μM | 30.0 μM | — | — |
| | acid | | | | | | | | | | | |
| | −NADPH | Average | 100 | 101 | 96.5 | 80.1 | 57.4 | 32.8 | 16.1 | 8.14 | 1.50 | 6.07 |
| | | SD | 9.82 | 1.68 | 1.29 | 2.26 | 4.96 | 0.795 | 1.75 | 0.593 | | |
| | +NADPH | Average | 100 | 88.9 | 74.4 | 36.8 | 14.9 | 9.24 | 5.66 | 3.84 | 0.247 | |
| | | SD | NA | 5.42 | 2.77 | 2.73 | 0.245 | 0.311 | 0.334 | 0.0850 | | |
| CYP2C19 | Ticlopidine | — | 0 μM | 0.0412 μM | 0.123 μM | 0.370 μM | 1.11 μM | 3.33 μM | 10.0 μM | 30.0 μM | — | — |
| | −NADPH | Average | 100 | 94.1 | 94.8 | 81.9 | 52.0 | 29.6 | 12.5 | 6.33 | 1.35 | 3.28 |
| | | SD | 2.40 | 3.29 | 6.76 | 11.5 | 1.58 | 4.08 | 2.00 | 0.222 | | |
| | +NADPH | Average | 100 | 94.9 | 79.5 | 57.1 | 21.3 | 10.1 | 5.00 | 3.72 | 0.412 | |
| | | SD | 4.07 | 8.22 | 3.72 | 1.69 | 1.46 | 2.44 | 0.312 | 0.561 | | |
| CYP2D6 | Paroxetine | — | 0 μM | 0.0412 μM | 0.123 μM | 0.370 μM | 1.11 μM | 3.33 μM | 10.0 μM | 30.0 μM | — | — |
| | −NADPH | Average | 100 | 97.3 | 95.8 | 88.6 | 76.4 | 62.0 | 41.6 | 28.0 | 5.73 | 14.0 |
| | | SD | 2.60 | 1.60 | 3.02 | 3.95 | 7.03 | 1.68 | 5.34 | 1.65 | | |
| | +NADPH | Average | 100 | 89.7 | 79.1 | 51.3 | 29.2 | 18.8 | 14.8 | 12.2 | 0.410 | |
| | | SD | 2.45 | 0.946 | 2.89 | 3.56 | 0.459 | 0.725 | 1.01 | 0.957 | | |
| CYP2A | Trolean- | — | 0 μM | 0.137 μM | 0.412 μM | 1.23 μM | 3.70 μM | 11.1 μM | 33.3 μM | 100 μM | — | — |
| (Midazo- | domycin | | | | | | | | | | | |
| lam) | −NADPH | Average | 100 | 101 | 93.1 | 84.9 | 74.9 | 64.7 | 41.0 | 25.3 | 19.2 | 11.5 |
| | | SD | 3.25 | 1.29 | 3.02 | 4.57 | 2.12 | 7.18 | 2.74 | 2.16 | | |
| | +NADPH | Average | 100 | 78.8 | 63.2 | 52.1 | 39.7 | 30.5 | 21.6 | 12.2 | 1.67 | |
| | | SD | 2.49 | 3.43 | 4.55 | 1.41 | 1.42 | 0.572 | 1.46 | 0.799 | | |
| CYP3A | Trolean- | — | 0 μM | 0.137 μM | 0.412 μM | 1.23 μM | 3.70 μM | 11.1 μM | 33.3 μM | 100 μM | — | — |
| (Testos- | domycin | | | | | | | | | | | |
| terone) | −NADPH | Average | 100 | 93.2 | 88.0 | 81.7 | 76.7 | 80.1 | 53.4 | 31.0 | 43.1 | 34.5 |
| | | SD | 6.50 | 4.59 | 5.31 | 1.30 | 4.30 | 12.3 | 2.92 | 2.20 | | |
| | +NADPH | Average | 100 | 69.7 | 61.1 | 44.6 | 37.4 | 34.8 | 20.1 | 13.7 | 1.25 | |
| | | SD | 4.35 | 2.60 | 4.97 | 2.81 | 3.93 | 5.12 | 2.59 | 1.10 | | |

^{a}When the enzyme activity was >50% of control at the highest inhibitor concentration, the IC₅₀ is expressed as >the highest concentration.
^{b}IC₅₀ shift = IC_{50 (-NADPH)}/IC_{50 (+NADPH)}. QC for positive control: IC₅₀ shift ≥3.
NA, not applicable (n = 2);
ND, not determined

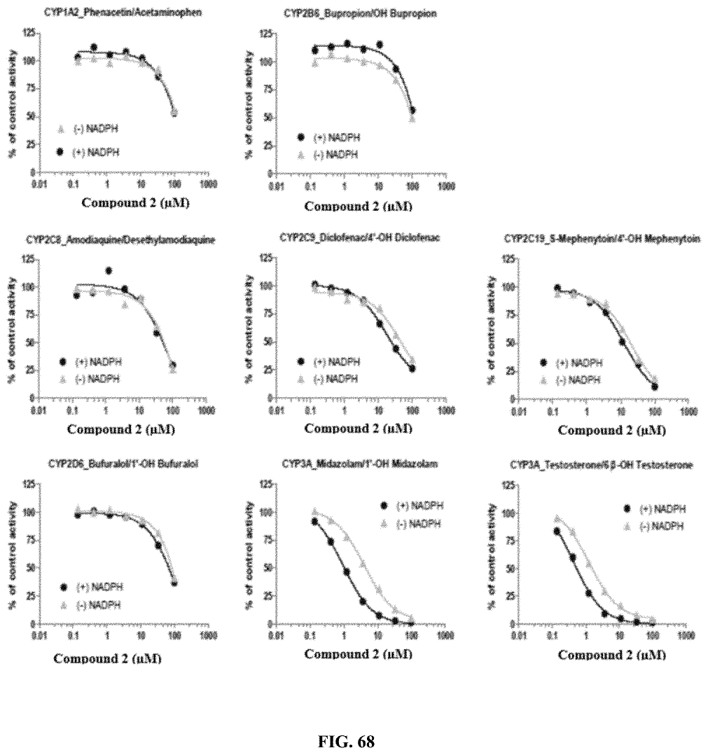

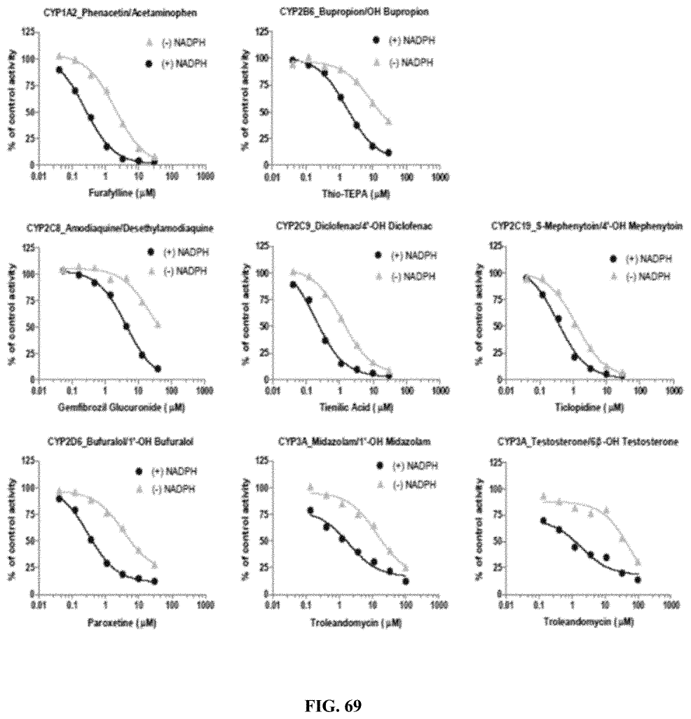

The IC₅₀ shift values of Compound 2 for CYP1A2, CYP2B6, CYP2C8, and CYP2D6 were less than the TDI threshold of 1.5 or not measurable (i.e., IC₅₀>100 μM), suggesting that Compound 2 was unlikely to be a time-dependent inhibitor of CYP1A2, CYP2B6, CYP2C8, and CYP2D6. The IC₅₀ shift values of Compound 2 were >1.5 for CYP2C9 (IC₅₀ shift=1.91), CYP2C19 (IC₅₀ shift=1.55), and CYP3A (IC₅₀ shift=4.27 with midazolam as the probe substrate and IC₅₀ shift=2.89 with testosterone as the probe substrate). The data suggested that Compound 2 was likely to be a time-dependent inhibitor of CYP3A, CYP2C9, and CYP2C19. FIG. 68 illustrates CYP Activity vs Compound 2 Concentration Curves. FIG. 69 illustrates CYP Activity vs Positive TDI Concentration Curves.

### Example 25: Binding of Compound 2 to Human, Rat, Mouse, Dog, and Monkey Plasma Proteins

The percent bound of Compound 2 was determined in human, rat, mouse, dog, and monkey plasma using equilibrium dialysis. Studies were carried out in mixed-gender human plasma, male Sprague-Dawley rat plasma, male CD-1 mouse plasma, male Beagle dog plasma, and male Cynomolgus monkey plasma collected on sodium heparin. A Rapid Equilibrium Dialysis (RED) device was used for all experiments. Stock solutions of Compound 2 and control compound were first prepared in DMSO. Aliquots of the DMSO solutions were dosed into 1.0 mL of plasma at a dosing concentration of 0.5 μM, 3 μM, 12 μM, 50 μM, and 200 μM for Compound 2 and 10 μM for the control compound, warfarin. Plasma (300 μL), containing Compound 2 or control compound, was loaded into three wells of the 96-well dialysis plate. Blank PBS (500 μL) was added to each corresponding receiver chamber. The device was then placed into an enclosed heated rocker that was pre-warmed to 37° C., and allowed to incubate for four hours. After 4 hours of incubation, both sides were sampled.

Aliquots (50 μL for donor, 200 μL for receiver) were removed from the chambers and placed into a 96-well plate. Plasma (50 μL) was added to the wells containing the receiver samples, and 200 μL of PBS was added to the wells containing the donor samples. Two volumes of acetonitrile were added to each well, and the plate was mixed and then centrifuged at 3,000 rpm for 10 minutes. Aliquots of the supernatant were removed, and analyzed by LCMS/MS. Calibration standards were prepared in a matched matrix and prepared similarly to the assay samples. Binding and recovery values were calculated as follows: % Bound=[(Concentration in Donor−Concentration in Receiver)/(Concentration in Donor)]×100; % Recovery=[(Concentration in Donor+Concentration in Receiver)/(Nominal Dosing Concentration)]×100. Results of the binding study are shown in TABLE 157. Warfarin binding acceptance criteria used for the study were Human: ≥98.0% bound; Rat: ≥95.0% bound; Mouse: ≥68.0% bound; Dog: ≥85.0% bound; Monkey: ≥98.0% bound.

TABLE 157

| | | Dosing | % Bound | | | | | % Recovery | | | | |
|---|---|---|---|---|---|---|---|---|---|---|---|---|
| Test Article | Species | Conc. (μM) | R1 | R2 | R3 | Mean (StDev) | | R1 | R2 | R3 | Mean (StDev) | |
| Compound 2 | Human | 0.5 | 99.4 | 99.4 | 99.5 | 99.4 | (0.0605) | 117 | 118 | 106 | 114 | (6.50) |
| | | 3 | 99.4 | 99.3 | 99.4 | 99.4 | (0.0624) | 112 | 113 | 107 | 111 | (3.10) |
| | | 12 | 99.2 | 99.2 | 99.3 | 99.2 | (0.0783) | 106 | 102 | 102 | 104 | (2.51) |
| | | 50 | 99.1 | 99.2 | 99.3 | 99.2 | (0.0971) | 96.1 | 92.9 | 96.2 | 95.0 | (1.89) |
| | | 200 | 99.0 | 99.8 | 98.9 | 98.9 | (0.109) | 99.2 | 88.3 | 81.5 | 89.7 | (8.94) |
| | Rat | 0.5 | 98.5 | 98.6 | 98.5 | 98.5 | (0.0867) | 105 | 105 | 99.0 | 103 | (3.44) |
| | | 3 | 98.4 | 98.3 | 98.3 | 98.3 | (0.0983) | 107 | 111 | 105 | 108 | (3.18) |
| | | 12 | 98.5 | 98.2 | 98.2 | 98.0 | (0.191) | 107 | 93.5 | 92.7 | 97.6 | (7.86) |
| | | 50 | 97.7 | 98.0 | 98.2 | 98.0 | (0.214) | 89.2 | 92.2 | 84.9 | 88.8 | (3.68) |
| | | 200 | 97.7 | 97.4 | 97.6 | 97.6 | (0.152) | 81.5 | 79.8 | 86.4 | 82.5 | (3.44) |
| | Mouse | 0.5 | 95.9 | 96.1 | 96.1 | 96.0 | (0.111) | 109 | 109 | 104 | 108 | (2.98) |
| | | 3 | 95.6 | 96.8 | 95.6 | 96.0 | (0.703) | 120 | 110 | 110 | 113 | (5.60) |
| | | 12 | 96.3 | 96.3 | 96.4 | 96.3 | (0.0622) | 97.3 | 93.8 | 96.3 | 95.8 | (1.81) |
| | | 50 | 89.1* | 94.3 | 95.5 | 94.9 | (0.901) | 96.9 | 94.2 | 95.2 | 95.5 | (1.37) |
| | | 200 | 95.7 | 95.6 | 94.7 | 95.3 | (0.570) | 89.5 | 88.0 | 83.3 | 86.9 | (3.24) |
| | Dog | 0.5 | 95.6 | 96.7 | 95.7 | 96.0 | (0.632) | 114 | 117 | 107 | 113 | (4.89) |
| | | 3 | 96.5 | 95.8 | 95.2 | 96.0 | (0.641) | 98.7 | 118 | 123 | 113 | (13.0) |
| | | 12 | 96.4 | 96.6 | 96.0 | 96.3 | (0.331) | 103 | 103 | 100 | 102 | (1.57) |
| | | 50 | 96.4 | 95.9 | 95.3 | 95.9 | (0.542) | 117 | 94.8 | 103 | 105 | (11.2) |
| | | 200 | 95.3 | 95.8 | 95.1 | 95.4 | (0.358) | 102 | 84.6 | 86.6 | 91.0 | (9.52) |
| | Monkey | 0.5 | 94.6* | 99.0 | 99.2 | 99.1 | (0.166) | 111 | 104 | 104 | 106 | (4.10) |
| | | 3 | 98.8 | 98.9 | 99.1 | 98.9 | (0.182) | 98.7 | 106 | 108 | 104 | (4.95) |
| | | 12 | 98.9 | 99.0 | 99.1 | 99.0 | (0.125) | 98.5 | 103 | 92.2 | 97.7 | (5.22) |
| | | 50 | 98.5 | 98.7 | 98.6 | 98.6 | (0.146) | 93.6 | 95.4 | 90.2 | 93.0 | (2.60) |
| | | 200 | 98.0 | 98.1 | 98.2 | 98.1 | (0.0648 | 81.6 | 87.6 | 86.6 | 85.3 | (3.23) |
| Warfarin | Human | | 98.9 | 99.2 | 99.0 | 99.0 | (0.143) | 91.9 | 90.4 | 87.6 | 90.0 | (2.19) |
| | Rat | | 98.9 | 98.8 | 98.7 | 98.9 | (0.112) | 90.5 | 90.1 | 83.6 | 88.1 | (3.89) |
| | Mouse | | 77.4 | 77.9 | 79.6 | 78.3 | (1.16) | 91.3 | 90.3 | 92.6 | 91.4 | (1.14) |
| | Dog | | 97.3 | 97.4 | 97.4 | 97.4 | (0.0621) | 83.1 | 88.1 | 82.5 | 84.5 | (3.10) |
| | Monkey | | 99.3 | 99.4 | 99.4 | 99.4 | (0.0574) | 98.3 | 97.5 | 95.0 | 96.9 | (1.73) |

*Data point was removed as an outlier, and excluded from the calculation of averages.

Liquid Chromatography was performed using 25 mM ammonium formate buffer, pH 3.5 as a mobile phase buffer; 90% water, 10% buffer as aqueous solution A; 90% acetonitrile, 10% buffer as organic solution B; and a flow rate of 0.7 mL/min. The total run time was 1.0 min; 1-10 μL autosampler injection volume; wash 1 was performed with water/methanol/2-propanol: 1/1/1 with 0.2% formic acid; and wash 2 was performed with 0.1% formic acid in water. The gradient program used is shown in TABLE 158. Mass spectrometry was performed using a PE SCIEX API4000; Turbo Ionspray interface; multiple reaction monitoring; and a 1.0 minute duration. TABLE 159 shows the MS settings used to obtain data. A summary of the results is shown in TABLE 160.

TABLE 158

| Time (min) | % A | % B |
|---|---|---|
| 0.00 | 99 | 1 |
| 0.65 | 1 | 99 |
| 0.75 | 1 | 99 |
| 0.80 | 99 | 1 |
| 1.00 | 99 | 1 |

TABLE 159

| Test Article | +/− | Q1 | Q3 | DP | EP | CE | CXP | IS |
|---|---|---|---|---|---|---|---|---|
| Compound 2 | + | 546.3 | 114.2 | 100 | 10 | 35 | 11 | 550 |

TEM: 500;
CAD: 7;
CUR: 30;
GS1: 50;
GS2: 50

TABLE 160

| | | Dosing Concentration | |
|---|---|---|---|
| Test Article | Species | (μM) | % Bound |
| Compound 2 | Human | 0.5 | 99.4 |
| | | 3 | 99.4 |
| | | 12 | 99.2 |
| | | 50 | 99.2 |
| | | 200 | 98.9 |
| | Rat | 0.5 | 98.5 |
| | | 3 | 98.3 |
| | | 12 | 98.3 |
| | | 50 | 98.0 |
| | | 200 | 97.6 |
| | Mouse | 0.5 | 96.0 |
| | | 3 | 96.0 |
| | | 12 | 96.3 |
| | | 50 | 94.9 |
| | | 200 | 95.3 |
| | Dog | 0.5 | 96.0 |
| | | 3 | 95.9 |
| | | 12 | 96.3 |
| | | 50 | 95.9 |
| | | 200 | 95.4 |
| | Monkey | 0.5 | 99.1 |
| | | 3 | 98.9 |
| | | 12 | 99.0 |
| | | 50 | 98.6 |
| | | 200 | 98.1 |

### Example 26: OAT1, OAT3, OCT2, OATP1B1, OATP1B3, MATE1, and MATE2K Substrate and Inhibition Assessment of Compound 2 Using Transfected HEK Cells

Experimental Procedure For Uptake Transporter Substrate Assessment: The substrate and inhibition potential of Compound 2 were determined for various uptake transporters (OAT1, OAT3, OCT2, OATP1B1, OATP1B3, MATE1, and MATE2K) using transfected HEK cells. Human embryonic kidney epithelial cells (HEK293) transfected with individual uptake transporters (OAT1, OAT3, OCT2, OATP1B1, OATP1B3, MATE1, and MATE2K) were used to assess the substrate potential of Compound 2 for the transporters. The cells were maintained in DMEM supplemented with 10% FBS, 1% NEAA, 4 mM L-glutamine, PEST (100 IU penicillin, 100 μg/mL streptomycin), 800 μg/mL G418, and 1 mM sodium pyruvate in a humidified incubator (37±1° C. 5±1% CO₂). The culture medium was changed three times weekly, and cell growth was observed by light microscopy. When the cells became confluent, the cells were harvested by trypsinization, and the collected cells were seeded onto plates for the uptake studies. The plates were placed in a humidified incubator (37±1° C. 5±1% CO₂). After 24-48 hours, each cell line was checked with a transporter-specific fluorescent marker compound to confirm the functionality of the transfected transporter. All experiments were conducted in duplicate (n=2). The cells were incubated for the indicated time in a humidified incubator (37° C. 5% CO₂). The dosing solution was prepared by diluting the test article stock solution in HBSSg. The uptake incubation was stopped by two washes with ice-cold HBSSg. The cells were lysed with 400 μL 75% acetonitrile and an aliquot of lysate was transferred to a 96-well deep block for analysis by LC-MS/MS. TABLE 161 shows the assay conditions of the study. Cell Batch Quality Control Results are shown in TABLES 162-168. Acceptance criterion was Ratio between HEK transfected and vector control>3.

TABLE 161

| | Probe Substrate | Assay Incubation |
|---|---|---|
| Cell Lines | (μM) | (minutes) |
| OAT1-transfected and vector control- | 0.3 and 5.0 | 2 and 10 |
| transfected cells | | |
| OAT3-transfected and vector control- | 0.3 and 5.0 | 2 and 10 |
| transfected cells | | |
| OCT2-transfected and vector control- | 0.3 and 5.0 | 2 and 10 |
| transfected cells | | |
| OATP1B1-transfected and vector | 0.3 and 5.0 | 2 and 10 |
| control-transfected cells | | |
| OATP1B3-transfected and vector | 0.3 and 5.0 | 2 and 10 |
| control-transfected cells | | |
| MATE1-transfected and vector | 0.3 and 5.0 | 2 and 10 |
| control-transfected cells | | |
| MATE2K-transfected and vector | 0.3 and 5.0 | 2 and 10 |
| control-transfected cells | | |

TABLE 162

| | Average Concentration | Ratio of 6′CF in |
|---|---|---|
| Cell Line | of 6′CF (μM) | OAT1/HEK Cells |
| HEK Vector Control | 0.00183 | 329 |
| OAT1-HEK | 0.601 | |

TABLE 163

| | Average Concentration | Ratio of 5′CF in |
|---|---|---|
| Cell Line | of 5′CF (μM) | OAT3/HEK Cells |
| HEK Vector Control | 0.0217 | 10.6 |
| OAT-HEK | 0.229 | |

TABLE 164

| | Average Concentration | Ratio of ASP in |
|---|---|---|
| Cell Line | of ASP (μM) | OCT2/HEK Cells |
| HEK Vector Control | 0.0167 | 16.4 |
| OCT2-HEK | 0.273 | |

TABLE 165

| | Average Concentration | Ratio of F-MTX in |
|---|---|---|
| Cell Line | of F-MTX (μM) | OATP1B3/HEK Cells |
| HEK Vector Control | 0.00573 | 19.9 |
| OCT2-HEK | 0.114 | |

TABLE 166

| | Average Concentration | Ratio of F-MTX in |
|---|---|---|
| Cell Line | of F-MTX (μM) | OATP1B3/HEK Cells |
| HEK Vector Control | 0.00573 | 35.7 |
| OATP1B3-HEL | 0.205 | |

TABLE 167

| | Average Concentration | Ratio of ASP in |
|---|---|---|
| Cell Line | of ASP (μM) | MATE1/HEK Cells |
| HEK Vector Control | 0.0174 | 20.7 |
| MATE1-HEK | 0.360 | |

TABLE 168

| | Average Concentration | Ratio of ASP in |
|---|---|---|
| Cell Line | of ASP (μM) | MATE2K/HEK Cells |
| HEK Vector | 0.0234 | 5.59 |
| Control | | |
| MATE2K-HEK | 0.131 | |

Data analysis was performed using the equation: Influx Rate=[(C_{S}×V_{S})/(C_{P}×V_{P})]/Experimental Duration, where C_{S}=Substrate concentration in the cell lysate in μM; V_{S}=Substrate assessment cell lysate volume in mL; C_{P}=Protein concentration in the cell lysate in mg/mL; V_{P}=Protein content determination cell lysate volume in mL; Influx Rate Ratio=IR_{TF}/IR_{VC}; IR_{TF}=Influx rate in transfected cells; and IR_{VC}=Influx rate in vector control cells.

Results for the uptake transporter substrate assessment are shown in TABLES 169 to 175. Substrate Assessment Criteria was based on Influx Rate Ratio≥2.0: Positive; and Influx Rate Ratio<2.0: Negative.

TABLE 169
OAT1

| | Incubation Time | | | | | | |
|---|---|---|---|---|---|---|---|
| | (min)/Dosing | | Influx Rate | | | Influx | |
| | Concentration | | (pmol/minute/mg/protein) | | | Rate | Substrate |
| Test Article | (μM) | Cell Line | R1 | R2 | AVG | Ratio | Assessment |
| Compound 2 | 2 min/0.3 μM | Vector | 0.0181 | 0.0150 | 0.0165 | 0.896 | Negative |
| | | Control | | | | | |
| | | OAT1 | 0.0146 | 0.0151 | 0.0148 | | |
| | 10 min/0.3 μM | Vector | 0.0105 | 0.00976 | 0.0101 | 0.745 | Negative |
| | | Control | | | | | |
| | | OAT1 | 0.00779 | 0.00734 | 0.00756 | | |
| | 2 min/5 μM | Vector | 0.273 | 0.298 | 0.286 | 0.843 | Negative |
| | | Control | | | | | |
| | | OAT1 | 0.238 | 0.244 | 0.241 | | |
| | 10 min/5 μM | Vector | 0.172 | 0.149 | 0.161 | 0.748 | Negative |
| | | Control | | | | | |
| | | OAT1 | 0.120 | 0.121 | 0.120 | | |
| PAH | 5 min/10 μM | Vector | 14.7 | 11.5 | 13.1 | 9.41 | Negative |
| | | Control | | | | | |
| | | OAT1 | 130 | 116 | 123 | | |

TABLE 170
OAT3

| | Incubation Time | | | | | | |
|---|---|---|---|---|---|---|---|
| | (min)/Dosing | | Influx Rate | | | Influx | |
| | Concentration | | (pmol/minute/mg/protein) | | | Rate | Substrate |
| Test Article | (μM) | Cell Line | R1 | R2 | AVG | Ratio | Assessment |
| Compound 2 | 2 min/0.3 μM | Vector | 0.0181 | 0.0150 | 0.0165 | 0.858 | Negative |
| | | Control | | | | | |
| | | OAT3 | 0.0137 | 0.0147 | 0.0142 | | |
| | 10 min/0.3 μM | Vector | 0.0105 | 0.00976 | 0.0101 | 0.664 | Negative |
| | | Control | | | | | |
| | | OAT3 | 0.00696 | 0.00651 | 0.00673 | | |
| | 2 min/5 μM | Vector | 0.273 | 0.298 | 0.286 | 0.798 | Negative |
| | | Control | | | | | |
| | | OAT3 | 0.226 | 0.230 | 0.228 | | |
| | 10 min/5 μM | Vector | 0.172 | 0.149 | 0.161 | 0.698 | Negative |
| | | Control | | | | | |
| | | OAT3 | 0.116 | 0.109 | 0.112 | | |
| Furosemide | 5 min/10 μM | Vector | 13.1 | 14.3 | 13.7 | 3.65 | Positive |
| | | Control | | | | | |
| | | OAT3 | 48.8 | 51.1 | 49.9 | | |

TABLE 171
OCT2

| | Incubation Time | | | | | | |
|---|---|---|---|---|---|---|---|
| | (min)/Dosing | | Influx Rate | | | Influx | |
| | Concentration | | (pmol/minute/mg/protein) | | | Rate | Substrate |
| Test Article | (μM) | Cell Line | R1 | R2 | AVG | Ratio | Assessment |
| Compound 2 | 2 min/0.3 μM | Vector | 0.0261 | 0.0257 | 0.0.259 | 1.17 | Negative |
| | | Control | | | | | |
| | | OCT2 | 0.0286 | 0.0318 | 0.0302 | | |
| | 10 min/0.3 μM | Vector | 0.0125 | 0.0126 | 0.0126 | 1.09 | Negative |
| | | Control | | | | | |
| | | OCT2 | 0.0144 | 0.0129 | 0.0137 | | |
| | 2 min/5 μM | Vector | 0.411 | 0.393 | 0.402 | 1.04 | Negative |
| | | Control | | | | | |
| | | OCT2 | 0.455 | 0.382 | 0.419 | | |
| | 10 min/5 μM | Vector | 0.214 | 0.216 | 0.215 | 1.15 | Negative |
| | | Control | | | | | |
| | | OCT2 | 0.250 | 0.244 | 0.247 | | |
| MPP | 2 min/5 μM | Vector | 29.5 | 19.0 | 24.3 | 16.6 | Positive |
| | | Control | | | | | |
| | | OCT2 | 404 | 402 | 403 | | |

TABLE 172
OATP1B1

| | Incubation Time | | | | | | |
|---|---|---|---|---|---|---|---|
| | (min)/Dosing | | Influx Rate | | | Influx | |
| | Concentration | | (pmol/minute/mg protein) | | | Rate | Substrate |
| Test Article | (PM) | Cell Line | R1 | R2 | AVG | Ratio | Assessment |
| Compound 2 | 2 min/0.3 μM | Vector | 0.0522 | 0.0238 | 0.0380 | 0.725 | Negative |
| | | Control | | | | | |
| | | OATPIB1 | 0.0267 | 0.0284 | 0.0275 | | |
| | 10 min./0.3 μM0 | Vector | 0.0127 | 0.0120 | 0.0124 | 0.968 | Negative |
| | | Control | | | | | |
| | | OATPIB1 | 0.0123 | 0.0116 | 0.0120 | | |
| | 2 min/5 μM | Vector | 0.417 | 0.456 | 0.437 | 1.04 | Negative |
| | | Control | | | | | |
| | | OATP1B1 | 0.475 | 0.429 | 0.452 | | |
| | 10 min/5 μM | Vector | 0.217 | 0.229 | 0.223 | 0.934 | Negative |
| | | Control | | | | | |
| | | OATP1B1 | 0.199 | 0.217 | 0.208 | | |
| Atorvastatin | 5 min/0.15 μM | Vector | 0.658 | 0.640 | 0.649 | 11.4 | Positive |
| | | Control | | | | | |
| | | OATPIB1 | 7.88 | 6.96 | 7.42 | | |

TABLE 173
OATP1B3

| | Incubation Time | | | | | | |
|---|---|---|---|---|---|---|---|
| | (min)/Dosing | | Influx Rate | | | Influx | |
| | Concentration | | (pmol/minute/mg protein) | | | Rate | Substrate |
| Test Article | (PM) | Cell Line | R1 | R2 | AVG | Ratio | Assessment |
| Compound 2 | 2 min/0.3 μM | Vector | 0.0522 | 0.0238 | 0.0380 | 0.639 | Negative |
| | | Control | | | | | |
| | | OATPIB3 | 0.0249 | 0.0237 | 0.0243 | | |
| | 10 min./0.3 μM | Vector | 0.0127 | 0.0120 | 0.0124 | 0.818 | Negative |
| | | Control | | | | | |
| | | OATPIB3 | 0.0102 | 0.0100 | 0.0101 | | |
| | 2 min/5 μM | Vector | 0.417 | 0.456 | 0.437 | 0.866 | Negative |
| | | Control | | | | | |
| | | OATP1B3 | 0.400 | 0.357 | 0.378 | | |
| | 10 min/5 μM | Vector | 0.217 | 0.229 | 0.223 | 0.744 | Negative |
| | | Control | | | | | |
| | | OATP1B3 | 0.170 | 0.161 | 0.166 | | |
| Atorvastatin | 5 min/0.15 μM | Vector | 0.658 | 0.640 | 0.649 | 7.55 | Positive |
| | | Control | | | | | |
| | | OATPIB3 | 5.15 | 4.65 | 4.90 | | |

TABLE 174
MATE1

| | Incubation Time | | | | | | |
|---|---|---|---|---|---|---|---|
| | (min)/Dosing | | Influx Rate | | | Influx | |
| | Concentration | | (pmol/minute/mg protein) | | | Rate | Substrate |
| Test Article | (PM) | Cell Line | R1 | R2 | AVG | Ratio | Assessment |
| Compound 2 | 2 min/0.3 μM | Vector | 0.0513 | 0.0554 | 0.0534 | 0.961 | Negative |
| | | Control | | | | | |
| | | MATE1 | 0.0525 | 0.0500 | 0.0513 | | |
| | 10 min./0.3 μM | Vector | 0.0247 | 0.02460 | 0.0247 | 1.02 | Negative |
| | | Control | | | | | |
| | | MATE1 | 0.0247 | 0.0256 | 0.0252 | | |
| | 2 min/5 μM | Vector | 0.896 | 0.988 | 0.942 | 0.898 | Negative |
| | | Control | | | | | |
| | | MATE1I | 0.910 | 0.782 | 0.846 | | |
| | 10 min/5 μM | Vector | 0.427 | 0.413 | 0.420 | 1.02 | Negative |
| | | Control | | | | | |
| | | MATE1 | 0.428 | 0.432 | 0.430 | | |
| Metformin | 5 min/0.15 μM | Vector | 50.1 | 44.9 | 47.5 | 13.3 | Positive |
| | | Control | | | | | |
| | | MATE1 | 609 | 650 | 630 | | |

TABLE 175
MATE2K

| | Incubation Time | | | | | | |
|---|---|---|---|---|---|---|---|
| | (min)/Dosing | | Influx Rate | | | Influx | |
| | Concentration | | (pmol/minute/mg protein) | | | Rate | Substrate |
| Test Article | (PM) | Cell Line | R1 | R2 | AVG | Ratio | Assessment |
| Compound 2 | 2 min/0.3 μM | Vector | 0.0198 | 0.0288 | 0.0243 | 0.887 | Negative |
| | | Control | | | | | |
| | | MATE2K | 0.0282 | 0.0149 | 0.0216 | | |
| | 10 min./0.3 μM | Vector | 0.00645 | 0.00764 | 0.00704 | 0.959 | Negative |
| | | Control | | | | | |
| | | MATE2K | 0.00658 | 0.00693 | 0.0676 | | |
| | 2 min/5 μM | Vector | 0.267 | 0.278 | 0.273 | 0.917 | Negative |
| | | Control | | | | | |
| | | MATE2K | 0.276 | 0.224 | 0.250 | | |
| | 10 min/5 μM | Vector | 0.107 | 0.0999 | 0.104 | 0.881 | Negative |
| | | Control | | | | | |
| | | MATE2K | 0.0960 | 0.0865 | 0.0913 | | |
| Metformin | 5 min/0.15 μM | Vector | 6.06 | 5.71 | 5.88 | 8.18 | Positive |
| | | Control | | | | | |
| | | MATE2K | 50.6 | 45.7 | 48.1 | | |

Experimental Procedure for Uptake Transporter Inhibition Assessment: Human embryonic kidney epithelial cells (HEK293) transfected with individual uptake transporters (OAT1, OAT3, OCT2, OATP1B1, OATP1B3, MATE1, MATE2K) were used to assess the inhibition potential of Compound 2 toward the transporters. The cells were maintained in DMEM supplemented with 10% FBS, 1% NEAA, 4 mM L-glutamine, PEST (100 IU penicillin, 100 μg/mL streptomycin), 800 μg/mL G418, and 1 mM sodium pyruvate in a humidified incubator (37±1° C. 5±1% CO₂). The culture medium was changed three times weekly, and cell growth was observed by light microscopy. When the cells became confluent, the cells were harvested by trypsinization and the collected cells were seeded onto plates for the uptake studies. The plates were placed in a humidified incubator (37±1° C. 5±1% CO₂). After 48 hours, each cell line was checked with a transporter-specific fluorescent marker compound to confirm the functionality of the transfected transporter.

The inhibition assay comprised the following steps: 1) incubation with probe substrate in the absence and presence of two concentrations of test article solution; 2) at the end of the incubation period, the incubation solution was carefully aspirated and the cells were rinsed twice with ice cold HBSSg buffer; 3) the cells were lysed with internal standard-containing lysis buffer; and 4) the lysates were collected for analysis of probe substrate concentration. The experimental conditions are summarized below in TABLE 176. All incubations were performed in duplicate. Cell Batch Quality Control Results are shown in TABLES 162-168. Acceptance criterion was Ratio between HEK transfected and vector control>3.

TABLE 176

| | Probe | Assay | Test Article |
|---|---|---|---|
| | Substrate | Incubation | Concentration |
| Cell Lines | (μM) | (minute) | (μM) |
| OAT1-transfected, | PAH (10) | 5 | 10 and 100 |
| and vector control- | | | |
| transfected cells | | | |
| OATS-transfected, | Furosemide (5) | 5 | |
| and vector control- | | | |
| transfected cells | | | |
| OCT2-transfected, | MPP+ (5) | 2 | |
| and vector control- | | | |
| transfected cells | | | |
| OATP1B1-transfected, | Atorvastatin (0.15) | 5 | |
| and vector control- | | | |
| transfected cells | | | |
| OATP1B3-transfected, | Atoniastatin (0.1) | 5 | |
| and vector control- | | | |
| transfected ce1is | | | |
| MATE1-transfected, | Metformin (50) | 10 | |
| and vector control- | | | |
| transfected cells | | | |
| MATE2K-transfected, | Metformin (50) | 10 | |
| and vector control- | | | |
| transfected cells | | | |

Data analysis was performed using the equation: Influx Rate=[(C_{S}×V_{S})/(C_{P}×V_{P})]/Experimental Duration, where C_{S}=Substrate concentration in the cell lysate in μM; V_{S}=Substrate assessment cell lysate volume in mL; C_{P}=Protein concentration in the cell lysate in mg/mL; V_{P}=Protein content determination cell lysate volume in mL; Influx Rate Ratio=IR_{TF}/IR_{VC}; IR_{TF}=Influx rate in transfected cells; and IR_{VC}=Influx rate in vector control cells. Percentage inhibition was performed using the equation: percentage inhibition=(1−[((IR_{TF})_{TA}−(IR_{VC})_{TA})/((IR_{TF})_{No TA}−(IR_{VC})_{No TA})])×100, where: IR: average influx rate (amount of substrate normalized to average protein content and incubation time); C_{S} is the substrate concentration in the cell lysate in μM; V_{S} is the substrate assessment cell lysate volume in mL; C_{P} is the protein concentration in the cell lysate in mg/mL; V_{P} is the protein content determination cell lysate volume in mL; VC: vector control-transfected cells; TF: transporter-transfected cells; and TA: test article.

Results for Uptake Transporter Inhibition Assessment are shown in TABLES 177 to 183. Inhibition Potential Classification was based on Percent inhibition≥50%: Positive; Percent inhibition<50%: Negative.

TABLE 177
OAT1

| | | Influx Rate | | | | |
|---|---|---|---|---|---|---|
| | Inhibitor | (pmol/minute/mg | | | | Inhibition |
| | Concentration | protein) | | | Percent | Potential |
| Call Line | (μM) | R1 | R2 | AVG | Inhibition | Classification |
| Vector | None | 14.7 | 11.5 | 13.1 | — | — |
| Control | Probenecid | 27.1 | 40.5 | 33.8 | — | — |
| | (100) | | | | | |
| | Compound 2 | 12.2 | 10.5 | 11.4 | — | — |
| | (10) | | | | | |
| | Compound 2 | 12.0 | 8.49 | 10.3 | — | — |
| | (100) | | | | | |
| OAT1- | None | 130 | 116 | 123 | — | — |
| transfeeted | Probenecid | 70.7 | 57.4 | 64.1 | 72.5 | Positive |
| HEK cells | (100) | | | | | |
| | Compound 2 | 120 | 117 | 119 | 2.56 | Negative |
| | (10) | | | | | |
| | Compound 2 | 113 | 116 | 114 | 5.48 | Negative |
| | (100) | | | | | |

TABLE 178
OAT3

| | | Influx Rate | | | | |
|---|---|---|---|---|---|---|
| | Inhibitor | (pmol/minute/mg | | | | Inhibition |
| | Concentration | protein) | | | Percent | Potential |
| Call Line | (μM) | R1 | R2 | AVG | Inhibition | Classification |
| Vector | None | 13.1 | 14.3 | 13.7 | — | — |
| Control | Probenecid | 11.1 | 9.14 | 10.1 | — | — |
| | (100) | | | | | |
| | Compound 2 | 12.9 | 12.3 | 12.6 | — | — |
| | (10) | | | | | |
| | Compound 2 | 9.26 | 8.19 | 8.72 | — | — |
| | (100) | | | | | |
| OAT3- | None | 48.8 | 51.1 | 49.9 | — | — |
| transfeeted | Probenecid | 19.1 | 21.5 | 20.3 | 72.0 | Positive |
| HEK cells | (100) | | | | | |
| | Compound 2 | 49.2 | 45.3 | 47.2 | 4.41 | Negative |
| | (10) | | | | | |
| | Compound 2 | 36.6 | 33.4 | 35.0 | 27.5 | Negative |
| | (100) | | | | | |

TABLE 179
OCT2

| | | Influx Rate | | | | |
|---|---|---|---|---|---|---|
| | Inhibitor | (pmol/minute/mg | | | | Inhibition |
| | Concentration | protein) | | | Percent | Potential |
| Call Line | (μM) | R1 | R2 | AVG | Inhibition | Classification |
| Vector | None | 29.5 | 19.0 | 24.3 | — | — |
| Control | Imipramine | 15.6 | 18.8 | 17.2 | — | — |
| | (300) | | | | | |
| | Compound 2 | 18.0 | 15.0 | 16.5 | — | — |
| | (10) | | | | | |
| | Compound 2 | 35.8 | 17.7 | 26.7 | — | — |
| | (100) | | | | | |
| OCT2- | None | 404 | 402 | 403 | — | — |
| transfeeted | Imipramine | 44.6 | 44.5 | 44.5 | 92.8 | Positive |
| HEK cells | (300) | | | | | |
| | Compound 2 | 303 | 311 | 307 | 23.3 | Negative |
| | (10) | | | | | |
| | Compound 2 | 356 | 376 | 366 | 10.5 | Negative |
| | (100) | | | | | |

TABLE 180
OATP1B1

| | | Influx Rate | | | | |
|---|---|---|---|---|---|---|
| | Inhibitor | (pmol/minute/mg | | | | Inhibition |
| | Concentration | protein) | | | Percent | Potential |
| Call Line | (μM) | R1 | R2 | AVG | Inhibition | Classification |
| Vector | None | 0.658 | 0.640 | 0.649 | — | — |
| Control | Rifamycin SV | 0.784 | 0.754 | 0.769 | — | — |
| | (10) | | | | | |
| | Compound 2 | 0.586 | 0.784 | 0.685 | — | — |
| | (10) | | | | | |
| | Compound 2 | 0.754 | 0.981 | 0.867 | — | — |
| | (100) | | | | | |
| OATP1B1-- | None | 7.88 | 6.96 | 7.42 | — | — |
| transfected | Rifamycin SV | 1.15 | 1.10 | 1.12 | 94.8 | Positive |
| HEK cells | (10) | | | | | |
| | Compound 2 | 4.99 | 4.70 | 4.84 | 38.6 | Negative |
| | (10) | | | | | |
| | Compound 2 | 1.06 | 1.16 | 1.11 | 96.4 | Negative |
| | (100) | | | | | |

TABLE 181
OATP1B3

| | | Influx Rate | | | | |
|---|---|---|---|---|---|---|
| | Inhibitor | (pmol/minute/mg | | | | Inhibition |
| | Concentration | protein) | | | Percent | Potential |
| Call Line | (μM) | R1 | R2 | AVG | Inhibition | Classification |
| Vector | None | 0.658 | 0.640 | 0.649 | — | — |
| Control | Rifamycin SV | 0.784 | 0.754 | 0.769 | — | — |
| | (10) | | | | | |
| | Compound 2 | 0.586 | 0.784 | 0.685 | — | — |
| | (10) | | | | | |
| | Compound 2 | 0.754 | 0.981 | 0.867 | — | — |
| | (100) | | | | | |
| OATP1B3- | None | 5.15 | 4.65 | 4.90 | — | — |
| transfected | Rifamycin SV | 1.13 | 0.853 | 0.991 | 94.8 | Positive |
| HEK cells | (10) | | | | | |
| | Compound 2 | 2.93 | 2.95 | 2.94 | 46.9 | Negative |
| | (10) | | | | | |
| | Compound 2 | 1.01 | 1.10 | 1.06 | 95.5 | Positive |
| | (100) | | | | | |

TABLE 182
MATE1

| | | Influx Rate | | | | |
|---|---|---|---|---|---|---|
| | Inhibitor | (pmol/minute/mg | | | | Inhibition |
| | Concentration | protein)μ | | | Percent | Potential |
| Call Line | (μM) | R1 | R2 | AVG | Inhibition | Classification |
| Vector | None | 50.1 | 44.9 | 47.5 | — | — |
| Control | Cimetidine | 24.3 | 33.2 | 28.7 | — | — |
| | (100) | | | | | |
| | Compound 2 | 57.8 | 36.5 | 47.2 | — | — |
| | (10) | | | | | |
| | Compound 2 | 32.0 | 56.9 | 44.4 | — | — |
| | (100) | | | | | |
| MATE1- | None | 6.09 | 650 | 630 | — | — |
| transfected | Cimetidine | 29.6 | 37.7 | 33.6 | 99.2 | Positive |
| HEK cells | (100) | | | | | |
| | Compound 2 | 198 | 223 | 211 | 71.9 | Positive |
| | (10) | | | | | |
| | Compound 2 | 78.7 | 79.1 | 78.9 | 94.1 | Positive |
| | (100) | | | | | |

TABLE 183
MATE2K

| | | Influx Rate | | | | |
|---|---|---|---|---|---|---|
| | Inhibitor | (pmol/minute/mg | | | | Inhibition |
| | Concentration | protein)μ | | | Percent | Potential |
| Call Line | (μM) | R1 | R2 | AVG | Inhibition | Classification |
| Vector | None | 6.06 | 5.71 | 5.88 | — | — |
| Control | Pyrimethamine | 4.01 | 4.55 | 4.28 | — | — |
| | (1) | | | | | |
| | Compound 2 | 3.71 | 3.45 | 3.58 | — | — |
| | (10) | | | | | |
| | Compound 2 | 2.66 | 3.48 | 3.07 | — | — |
| | (100) | | | | | |
| | None | 50.6 | 45.7 | 48.1 | — | — |
| MATE2K- | Pyrimethamine | 8.02 | 6.21 | 7.11 | 93.3 | Positive |
| transfected | (1) | | | | | |
| HEK cells | Compound 2 | 38.5 | 46.3 | 42.4 | 8.15 | Negative |
| | (10) | | | | | |
| | Compound 2 | 10.2 | 10.9 | 10.5 | 82.3 | Positive |
| | (100) | | | | | |

Compound 2 was not be a substrate of any of the tested transporters OAT1, OAT3, OCT2, OATP1B1, OATP1B3, MATE1, or MATE2K under all conditions tested. For the inhibition potential assessment of Compound 2, the compound was clearly demonstrated to not be a substrate of the OAT1, OAT3, and OCT2 transporters. Compound 2 was demonstrated to be an inhibitor of the MATE1 transporter at both the 10 μM and 100 μM dosing concentrations. For the remaining transporters (OATP1B1, OATP1B3, and MATE2K), Compound 2 showed significant inhibition at the 100 μM dosing concentration, but lesser or no inhibition at the 10 μM dosing concentration. TABLE 184 summarizes uptake transporter substrate assessments. TABLE 185 summarizes uptake transporter mm tutor assessments.

TABLE 184

| Test | Conc | | | | | | | |
|---|---|---|---|---|---|---|---|---|
| Article | (μM) | OAT1 | OAT3 | OCT2 | OATP1B1 | OATP1B3 | MATE1 | MATE2K |
| Compound 2 | 0.3 | Negative | Negative | Negative | Negative | Negative | Negative | Negative |
| | 5 | Negative | Negative | Negative | Negative | Negative | Negative | Negative |

TABLE 185

| Test | Cone | | | | | | | |
|---|---|---|---|---|---|---|---|---|
| Article | (μM) | OAT1 | OAT3 | OCT2 | OATP1B1 | OATP1B3 | MATE1 | MATE2K |
| Compound 2 | 10 | Negative | Negative | Negative | Negative | Negative | Positive | Negative |
| Compound 2 | 100 | Negative | Negative | Negative | Positive | Positive | Positive | Positive |

Liquid Chromatography and mass spectrometry Were performed using the procedure described in EXAMPLE 21.

### Example 27: Measurement of Compound 2 and Metabolites in Rat Plasma

The abundance of the major metabolites of Compound 2 following oral administration of Compound 2 at 50 mg/kg in rat was determined. The proposed metabolite pathway was determined in vitro, and levels of potential metabolites were measured by LC/MS-MS in rat plasma remaining from the dose rising 10-day repeat dose study in rats. Six potential metabolites were quantified in the low (50 mg/kg) single dose rats. Male (N=6) and female (N=6) Sprague Dawley rats were administered Compound 2 at 50 mg/kg orally (PO) for ten days (QD×10) and plasma was harvested via the tail vein at 1, 2, 4, 8, and 24 h post the first dose and 1, 2, 4, 8, 24, 48, 72 and 96 h post the tenth dose. Rats were rotated between timepoints to form a composite PK curve. Test Article, Compound 2: A 20 μL aliquot was protein precipitated with 200 μL IS solution (100 ng/mL Labetalol, 100 ng/mL Tolbutamide, and 100 ng/mL Diclofenac in ACN), and the mixture was vortex-mixed and centrifuged at 4000 rpm for 15 min at 4° C. A 100 μL aliquot of the supernatant was transferred to the sample plate and mixed with 100 μL of water, then the plate was shaken at 800 rpm for 10 min. 0.5-2 μL supernatant was then injected into a Triple Quad 6500+ for LC-MS/MS analysis. The standard curve was generated at 1-3000 ng/mL for the compounds in rat plasma (EDTA-K2).

Compound 3 is a metabolite of Compound 2 that lacks a methyl group. Compound 4 is a metabolite of Compound 2 that is an oxide of Compound 2. Compound 5 is a metabolite of Compound 2 that is an oxide of Compound 2, and a diastereomer of Compound 4. Compound 6 is a metabolite of Compound 2 that includes HCl. Compound 7 is a metabolite of Compound 2 that is lacking a methyl group. Compound 8 is a metabolite of Compound 2 that has an internally cyclized bicyclic group.

The male and female PK parameters were averaged together since there was <2-fold variation between the genders. All values deemed below the level of quantification (BLQ) were excluded from the PK parameters calculations. TABLE 186 shows calculated concentrations (ng/mL) of Compound 2 and metabolites in rats following a 50 mg/kg QD×1 dose. TABLE 187 shows average concentrations of Compound 2 and metabolites in rats following a 50 mg/kg QD×1 Dose. TABLE 188 shows calculated concentrations (ng/mL) of Compound 2 and Metabolites in rats following 50 mg/kg QD×10 Dosing. TABLE 189 shows average concentrations of Compound 2 and metabolites in rats following 50 mg/kg QD×10 Dosing.

TABLE 186

| Timepoint | Dilution | | | | | | | |
|---|---|---|---|---|---|---|---|---|
| (h) | Factor | Compound 2 | Compound 7 | Compound 3 | Compound 8 | Compound 4 | Compound 5 | Compound 6 |
| 1 | 20 | 2470 | 1.36 | 23.0 | 16.5 | 34.7 | 407 | 4.72 |
| 1 | 20 | 1670 | 1.19 | 28.1 | 13.4 | 28.5 | 342 | 3.58 |
| 1 | 20 | 1860 | 1.33 | 20.5 | 8.05 | 58.3 | 649 | 5.32 |
| 1 | 20 | 2550 | 1.49 | 25.4 | 22.8 | 44.0 | 486 | 12.0 |
| 1 | 20 | 2190 | 1.39 | 22.5 | 22.6 | 31.4 | 311 | 8.96 |
| 1 | 20 | 3140 | 1.44 | 24.0 | 28.5 | 52.4 | 576 | 15.4 |
| 2 | 20 | 4490 | 4.12 | 37.9 | 88.9 | 98.8 | 1050 | 19.3 |
| 2 | 20 | 3800 | 1.95 | 26.4 | 36.3 | 56.5 | 561 | 14.3 |
| 2 | 20 | 3440 | 1.67 | 26.8 | 32.5 | 57.4 | 617 | 13.2 |
| 2 | 20 | 5100 | 1.93 | 37.1 | 78.0 | 65.7 | 665 | 27.6 |
| 2 | 20 | 3870 | 1.81 | 33.0 | 62.0 | 58.2 | 630 | 17.3 |
| 2 | 20 | 3860 | 1.70 | 35.7 | 51.5 | 53.3 | 572 | 13.8 |
| 4 | 20 | 4680 | 2.46 | 46.7 | 84.8 | 72.2 | 732 | 27.3 |
| 4 | 20 | 3280 | 2.66 | 61.2 | 81.1 | 73.2 | 850 | 16.7 |
| 4 | 20 | 3750 | 2.27 | 40.8 | 34.9 | 96.5 | 1120 | 17.1 |
| 4 | 20 | 4920 | 1.68 | 47.9 | 38.5 | 63.9 | 738 | 19.3 |
| 4 | 20 | 4860 | 1.81 | 47.3 | 52.9 | 69.0 | 774 | 22.5 |
| 4 | 20 | 5040 | 2.04 | 39.1 | 58.3 | 74.3 | 861 | 25.3 |
| 8 | 20 | 5390 | 3.52 | 72.5 | 77.5 | 86.8 | 941 | 17.3 |
| 8 | 20 | 4180 | 2.72 | 65.3 | 46.5 | 66.7 | 742 | 20.4 |
| 8 | 20 | 6020 | 2.19 | 69.4 | 86.3 | 79.4 | 901 | 16.8 |
| 8 | 20 | 5660 | 2.24 | 86.3 | 203 | 71.8 | 752 | 37.0 |
| 8 | 20 | 4350 | 2.20 | 63.8 | 87.6 | 72.4 | 872 | 28.8 |
| 8 | 20 | 5320 | 2.11 | 77.0 | 73.8 | 63.5 | 696 | 28.8 |
| 24 | 20 | 850 | BLQ | 46.5 | 2.71 | 17.9 | 218 | 2.03 |
| 24 | 20 | 384 | BLQ | 47.1 | 1.20 | 11.4 | 144 | 1.19 |
| 24 | 20 | 475 | BLQ | 40.3 | 1.36 | 18.4 | 208 | 1.50 |
| 24 | 20 | 774 | BLQ | 32.8 | 2.31 | 24.0 | 266 | 6.26 |
| 24 | 20 | 1190 | BLQ | 38.3 | 2.43 | 22.5 | 246 | 7.89 |
| 24 | 20 | 515 | BLQ | 18.4 | 1.25 | 13.1 | 151 | 3.35 |

TABLE 187

| | | Average | |
|---|---|---|---|
| | Timepoint | Concentration | Standard |
| Compound | (h) | (ng/mL) | Deviation |
| Compound 2 | 1 | 2313.33 | 528.87 |
| | 2 | 4093.33 | 597.99 |
| | 4 | 4421.67 | 727.20 |
| | 8 | 5153.33 | 732.71 |
| | 24 | 698.00 | 300.92 |
| Compound 7 | 1 | 1.37 | 0.10 |
| | 2 | 2.20 | 0.95 |
| | 4 | 2.15 | 0.38 |
| | 8 | 2.50 | 0.55 |
| | 24 | BLQ | BLQ |
| Compound 3 | 1 | 23.92 | 2.62 |
| | 2 | 32.82 | 5.10 |
| | 4 | 47.17 | 7.79 |
| | 8 | 72.38 | 8.34 |
| | 24 | 37.23 | 10.66 |
| Compound 8 | 1 | 18.64 | 7.41 |
| | 2 | 58.20 | 22.51 |
| | 4 | 58.42 | 20.93 |
| | 8 | 95.78 | 54.59 |
| | 24 | 1.88 | 0.68 |
| Compound 4 | 1 | 41.55 | 12.04 |
| | 2 | 64.98 | 17.06 |
| | 4 | 74.85 | 11.25 |
| | 8 | 73.43 | 8.51 |
| | 24 | 17.88 | 4.98 |
| Compound 5 | 1 | 461.83 | 133.24 |
| | 2 | 682.50 | 184.06 |
| | 4 | 845.83 | 145.02 |
| | 8 | 817.33 | 99.95 |
| | 24 | 205.50 | 49.43 |
| Compound 6 | 1 | 8.33 | 4.66 |
| | 2 | 17.58 | 5.43 |
| | 4 | 21.37 | 4.39 |
| | 8 | 24.85 | 8.01 |
| | 24 | 3.70 | 2.76 |

BLQ = Below the lower limit of quantitation

TABLE 188

| Timepoint | Dilution | | | | | | | |
|---|---|---|---|---|---|---|---|---|
| (h) | Factor | Compound 2 | Compound 7 | Compound 3 | Compound 8 | Compound 4 | Compound 5 | Compound 6 |
| 1 | 20 | 1650 | 1.46 | 40.9 | 6.52 | 34.5 | 434 | 5.40 |
| 1 | 20 | 1620 | 1.42 | 67.1 | 10.4 | 45.1 | 580 | 6.58 |
| 1 | 20 | 1350 | 1.49 | 40.7 | 6.59 | 46.0 | 539 | 5.09 |
| 1 | 20 | 6840 | 1.92 | 68.7 | 46.7 | 64.9 | 753 | 22.8 |
| 1 | 20 | 5710 | 1.65 | 57.0 | 24.4 | 67.1 | 738 | 19.6 |
| 1 | 20 | 3900 | 1.25 | 33.8 | 16.5 | 48.8 | 532 | 14.5 |
| 2 | 20 | 2640 | 1.81 | 47.7 | 14.6 | 42.8 | 513 | 7.57 |
| 2 | 20 | 2730 | 2.19 | 55.6 | 17.1 | 51.0 | 639 | 11.1 |
| 2 | 20 | 3900 | 1.99 | 71.9 | 27.6 | 62.7 | 761 | 14.1 |
| 2 | 20 | 4440 | 1.41 | 43.2 | 38.9 | 54.2 | 625 | 21.2 |
| 2 | 20 | 5310 | 1.79 | 37.8 | 57.5 | 59.7 | 671 | 25.5 |
| 2 | 20 | 4620 | 1.66 | 46.0 | 24.2 | 52.9 | 616 | 18.6 |
| 4 | 20 | 4750 | 2.46 | 59.2 | 39.0 | 66.1 | 789 | 15.2 |
| 4 | 20 | 3820 | 2.18 | 77.7 | 39.4 | 54.7 | 675 | 11.6 |
| 4 | 20 | 3870 | 2.20 | 49.0 | 22.5 | 73.4 | 859 | 12.5 |
| 4 | 20 | 8730 | 2.42 | 86.8 | 92.1 | 93.1 | 1010 | 45.8 |
| 4 | 20 | 7600 | 2.00 | 64.6 | 65.1 | 83.9 | 887 | 33.9 |
| 4 | 20 | 3220 | 1.30 | 40.9 | 19.9 | 58.7 | 701 | 20.5 |
| 8 | 20 | 2930 | 2.31 | 76.1 | 29.5 | 53.2 | 619 | 12.7 |
| 8 | 20 | 4260 | 2.45 | 92.6 | 35.5 | 61.4 | 737 | 17.4 |
| 8 | 20 | 3740 | 2.79 | 121 | 57.2 | 69.2 | 807 | 20.4 |
| 8 | 20 | 6460 | 1.81 | 76.0 | 121 | 67.5 | 812 | 30.9 |
| 8 | 20 | 7030 | 2.20 | 77.7 | 135 | 73.1 | 881 | 31.8 |
| 8 | 20 | 6470 | 2.21 | 70.6 | 67.6 | 65.4 | 772 | 24.9 |
| 24 | 20 | 756 | BLQ | 43.9 | 1.63 | 17.1 | 216 | 1.87 |
| 24 | 20 | 1030 | 1.07 | 104 | 2.61 | 29.4 | 376 | 4.06 |
| 24 | 20 | 416 | BLQ | 44.6 | 2.12 | 19.7 | 234 | 2.02 |
| 24 | 20 | 2290 | 1.06 | 52.2 | 10.6 | 38.9 | 486 | 9.20 |
| 24 | 20 | 2440 | 1.16 | 63.9 | 7.70 | 40.7 | 477 | 11.3 |
| 24 | 20 | 2260 | BLQ | 31.5 | 8.00 | 40.1 | 484 | 8.91 |
| 48 | 20 | 21.1 | BLQ | 3.82 | BLQ | BLQ | 6.57 | BLQ |
| 48 | 20 | 41.5 | BLQ | 5.49 | 1.01 | 1.97 | 22.4 | BLQ |
| 48 | 20 | 34.7 | BLQ | 7.13 | BLQ | 1.33 | 13.6 | BLQ |
| 48 | 20 | 179 | BLQ | 8.94 | 1.21 | 4.63 | 55.0 | BLQ |
| 48 | 20 | 74.7 | BLQ | 5.09 | BLQ | 2.28 | 27.3 | BLQ |
| 48 | 20 | 97.1 | BLQ | 7.63 | BLQ | 2.67 | 31.2 | BLQ |
| 72 | 20 | BLQ | BLQ | 1.46 | BLQ | BLQ | 3.25 | BLQ |
| 72 | 20 | BLQ | BLQ | 2.10 | BLQ | BLQ | 2.30 | BLQ |
| 72 | 40 | BLQ | BLQ | 1.16 | BLQ | BLQ | BLQ | BLQ |
| 72 | 4 | 12.5 | BLQ | BLQ | BLQ | BLQ | BLQ | BLQ |
| 72 | 4 | 10.00 | BLQ | BLQ | BLQ | BLQ | BLQ | BLQ |
| 72 | 4 | 62.1 | BLQ | BLQ | BLQ | BLQ | 17.3 | BLQ |
| 96 | 20 | BLQ | BLQ | BLQ | BLQ | BLQ | BLQ | BLQ |
| 96 | 20 | BLQ | BLQ | BLQ | BLQ | BLQ | 1.27 | BLQ |
| 96 | 1 | 1.94 | BLQ | BLQ | BLQ | BLQ | BLQ | BLQ |
| 96 | 10 | BLQ | BLQ | BLQ | BLQ | BLQ | BLQ | BLQ |
| 96 | 4 | BLQ | BLQ | BLQ | BLQ | BLQ | BLQ | BLQ |
| 96 | 4 | BLQ | BLQ | BLQ | BLQ | BLQ | BLQ | BLQ |

TABLE 189

| | | Average | |
|---|---|---|---|
| Compound or | Timepoint | Concentration | Standard |
| Metabolite | (h) | (ng/mL) | Deviation |
| Compound 2 | 1 | 3511.67 | 2357.04 |
| | 2 | 3940.00 | 1071.91 |
| | 4 | 5331.67 | 2276.54 |
| | 8 | 5148.33 | 1714.73 |
| | 24 | 1532.00 | 897.62 |
| | 48 | 74.68 | 58.23 |
| | 72 | 28.20 | 29.38 |
| | 96 | 1.94 | BLQ |
| Compound 7 | 1 | 1.53 | 0.23 |
| | 2 | 1.81 | 0.27 |
| | 4 | 2.09 | 0.42 |
| | 8 | 2.30 | 0.32 |
| | 24 | 1.10 | 0.06 |
| | 48 | BLQ | BLQ |
| | 72 | BLQ | BLQ |
| | 96 | BLQ | BLQ |
| Compound 3 | 1 | 51.37 | 14.91 |
| | 2 | 50.37 | 12.05 |
| | 4 | 63.03 | 17.23 |
| | 8 | 85.67 | 18.83 |
| | 24 | 56.68 | 25.51 |
| | 48 | 6.35 | 1.88 |
| | 72 | 1.57 | 0.48 |
| | 96 | BLQ | BLQ |
| Compound 8 | 1 | 18.52 | 15.39 |
| | 2 | 29.98 | 15.99 |
| | 4 | 46.33 | 27.62 |
| | 8 | 74.30 | 44.08 |
| | 24 | 5.44 | 3.79 |
| | 48 | 1.11 | 0.14 |
| | 72 | BLQ | BLQ |
| | 96 | BLQ | BLQ |
| Compound 4 | 1 | 51.07 | 12.56 |
| | 2 | 53.88 | 6.98 |
| | 4 | 71.65 | 14.83 |
| | 8 | 64.97 | 6.95 |
| | 24 | 30.98 | 10.61 |
| | 48 | 2.58 | 1.25 |
| | 72 | BLQ | BLQ |
| | 96 | BLQ | BLQ |
| Compound 5 | 1 | 596.00 | 125.43 |
| | 2 | 637.50 | 80.62 |
| | 4 | 820.17 | 125.11 |
| | 8 | 771.33 | 88.70 |
| | 24 | 378.83 | 126.24 |
| | 48 | 26.01 | 16.82 |
| | 72 | 7.62 | 8.40 |
| | 96 | 1.27 | BLQ |
| Compound 6 | 1 | 12.33 | 7.75 |
| | 2 | 16.35 | 6.66 |
| | 4 | 23.25 | 13.75 |
| | 8 | 23.02 | 7.59 |
| | 24 | 6.23 | 4.08 |
| | 48 | BLQ | BLQ |
| | 72 | BLQ | BLQ |
| | 96 | BLQ | BLQ |

BLQ = Below the lower limit of quantitation

PK of Compound 2: Following single dose oral administration of Compound 2 at 50 mg/kg in Sprague Dawley rats, the area under the plasma concentration-time curve from time zero to the 24 h timepoint (AUC₀₋₂₄) was 78,836 ng·h/mL. The AUC₀₋₂₄ for metabolite Compound 5 was 13,840 ng·h/mL, representing 17.6% of the parent Compound 2 AUC₀₋₂₄. No other tested metabolite was determined to be more 1.6% of the parent Compound 2 AUC₀₋₂₄.

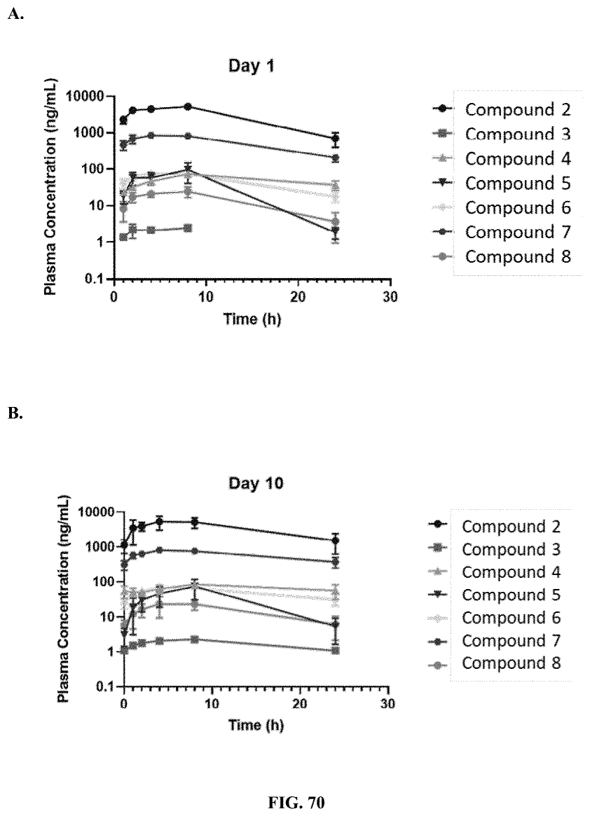

Repeat 10 day administration of Compound 2 at 50 mg/kg resulted in an AUC₀₋ₗₐₛₜ of 109,953 ng·h/mL. Compound 5 was again found to be the metabolite with the largest percentage of parent compound, at 18.1%, with an AUC₀₋ₗₐₛₜ of 19,943 ng·h/mL. Compound 3 was found to be 2.2% of parent compound with an AUC₀₋ₗₐₛₜ of 2452 ng·h/mL. No other metabolite was found to be more than 1.5% of parent AUC₀₋ₗₐₛₜ. TABLE 190 shows day 1 and 10 exposure of compound 2 and potential metabolites in rat following 50 mg/kg QD×10 dose. FIG. 70 PANEL A and PANEL B illustrate Day 1 and 10 PK Profile of Compound 2 and Metabolites 0-24 h in Rats Following 50 mg/kg QD×10 Dose.

TABLE 190

| | Day 1 | | Day 10 | |
|---|---|---|---|---|
| | AUC₀₋₂₄ | % of | AUC₀₋ₗₐₛₜ | % of |
| Compound Number | (ng · h/mL) | Parent | (ng · h/mL) | Parent |
| Compound 2 (Parent) | 78836 | 100.0 | 109953 | 100.0 |
| Compound 3 | 1236 | 1.6 | 2452 | 2.2 |
| Compound 4 | 1241 | 1.6 | 1620 | 1.5 |
| Compound 5 | 13840 | 17.6 | 19943 | 18.1 |
| Compound 6 | 377 | 0.5 | 389 | 0.4 |
| Compound 7 | 16 | 0.0 | 28 | 0.0 |
| Compound 8 | 1254 | 1.6 | 1021 | 0.9 |

### Example 28: Half-Life and Clearance of Compound 2 Metabolite

Mixed-gender human cryopreserved hepatocytes, male Sprague-Dawley rat cryopreserved hepatocytes, male CD-1 mouse cryopreserved hepatocytes, male beagle dog cryopreserved hepatocytes, and male cynomolgus monkey cryopreserved hepatocytes were used for the study. The hepatocytes were thawed, pooled into Krebs Henseleit buffer (KHB, pH 7.4), and kept on ice prior to the experiments. The hepatocyte suspension was equilibrated in a shaking water bath at 37° C. for 3 minutes, and then the reaction was initiated by spiking Compound 2 into the hepatocyte suspension (1.5×10⁶ cells/mL) at final Compound 2 concentrations of 1 μM and 50 μM. Each experiment was performed in triplicate. The final DMSO content in the incubation mixture was ≤0.1%. The reaction mixture was incubated in a shaking water bath at 37° C. Positive controls, testosterone (1 μM) and 7-hydroxycoumarin (7-HC) (100 μM), were performed in parallel to confirm the activity of the hepatocytes. Aliquots of the Compound 2 were withdrawn (n=1) at 0 and 120 minutes. Aliquots of testosterone were withdrawn (n=1) at 0, 5, 15, 30, 60, and 120 minutes. Aliquots of 7-HC were withdrawn (n=1) at 0 and 15 minutes. The reaction was immediately terminated by adding three volumes of ice-cold MeCN containing IS. The samples were then mixed and spun by centrifuge to precipitate proteins. An aliquot of the supernatant was then diluted with water. Calibration standards for the analysis of 7-HC metabolites were prepared in matched matrix. Compound 2 and testosterone samples were analyzed without calibration standards. All samples were analyzed by LC-MS/MS. The peak area response ratio (PARR) vs. IS was compared to the PARR at time 0 to determine the percent remaining at each time point. Half-lives and clearance values were calculated using GraphPad software, fitting to a single-phase exponential decay equation. TABLE 191 shows the half-lives and CLᵢₙₜ of Compound 2 in human, rat, mouse, dog, and monkey hepatocytes.

TABLE 191

| | | | Half-life |
|---|---|---|---|
| | Half- | CLᵢₙₜ | Acceptance |
| | life | (mL/min/10⁶ | Criteria |
| Species | (min) | cells) | (min) |
| Human | 4.28 | 0.108 | <5 |
| Rat | 1.14 | 0.405 | <5 |
| Mouse | 2.55 | 0.181 | <5 |
| Dog | 6.88 | 0.0672 | <10 |
| Monkey | 3.08 | 0.15 | <5 |

TABLE 192

| | | Formation | Acceptable |
|---|---|---|---|
| | | Rate | Range |
| | | (pmol/min/10⁶ | (pmol/min/10⁶ |
| Species | Analyte | cells) | cells) |
| Human | 7-HC-G | 316 | >50 |
| | 7-HC-S | 7.74 | >1 |
| Rat | 7-HC-G | 253 | >25 |
| | 7-HC-S | 34 | >5 |
| Mouse | 7-HC-G | 234 | >10 |
| | 7-HC-S | 6.36 | >1 |
| Dog | 7-HC-G | 301 | >50 |
| | 7-HC-S | 24.5 | >5 |
| Monkey | 7-HC-G | 279 | >50 |
| | 7-HC-S | 13.3 | >5 |

7-HC-G: 7-hydroxycoumarin glucuronide;
7-HC-S: 7-hydroxycoumarin sulfate

The LC-HRAMS method for metabolite profiling was performed with a Dionex XR3000 quaternary solvent HPLC system equipped with column compartment thermostat (set to +40° C.) for chromatographic separation. A PFP-C18, 3.0 μm, 100×2.1 mm column; 0.1% AcOH in water mobile phase A; and ACN mobile phase B were used with the gradient method and flow conditions of TABLE 193 and TABLE 194.

TABLE 193

| Time (minutes) | % B | Flow (mL/min) |
|---|---|---|
| 0 | 2 | 0.3 |
| 20 | 15 | 0.3 |
| 38 | 100 | 0.3 |
| 40 | 100 | 0.35 |
| 40.1 | 2 | 0.4 |
| 43 | 2 | 0.35 |
| 470 | 2 | 0.3 |

TABLE 194

| Time (minutes) | % B | Flow (mL/min) |
|---|---|---|
| 0 | 5 | 0.3 |
| 30 | 45 | 0.3 |
| 38 | 100 | 0.3 |
| 40 | 100 | 0.35 |
| 40.1 | 5 | 0.4 |
| 43 | 5 | 0.35 |
| 470 | 5 | 0.3 |

An LTQ-Orbitrap XL hybrid mass spectrometer was equipped with an HESI-II probe. The common settings included: Vaporizer temperature of +275° C.; capillary temperature of +275° C.; gases (arbitrary units): Sheath 45; Auxiliary: 15; Sweep 8; HRAMS survey scan of 150-900 Th; resolution in HRAMS of 60000; resolution in targeted HRAMSⁿ scans of 15000; MS/MS isolation width of 2 u; default manufacturer settings were used for automatic gain control; normalized collision energy in MS/MS (CID) of 35%; normalized high energy collision in MSⁿ (HCD) of 25%; positive detection mode; positive electrospray ionization V=+3.5 kV; capillary voltage +35 V; and tube lenses voltage of 110 V.

LC-MS/MS methods for metabolic stability were conducted using the method described in EXAMPLE 20. MS spectra were obtained using a PE SCIEX API 4000; Turbo Ionspray interface; multiple reaction monitoring mode; and a 1 min method. TABLE 195 shows the MS settings used to obtain data.

TABLE 195

| Test Article | +/− | Q1 | Q3 | DP | EP | CE | CXP | ISV |
|---|---|---|---|---|---|---|---|---|
| 0 | + | 546.3 | 114.2 | 100 | 10 | 35 | 11 | 5500 |

### Example 29: Cytochrome P450 Reaction Phenotyping of Compound 2 Using Human Recombinant CYP Enzymes

Cytochrome P450 (CYP) reaction phenotyping of Compound 2 was evaluated using human recombinant CYP enzymes (hrCYPs) by an in vitro intrinsic clearance (CLᵢₙₜ) approach. Compound 2 (1 μM) was incubated with individual hrCYPs (20 pmol CYP/mL) in phosphate buffer (100 mM, pH 7.4) containing MgCl₂ (5 mM) and NADPH (1 mM). The test article (TA), Compound 2, was (10 mM) was prepared in dimethyl sulfoxide (DMSO) and diluted using methanol to create a stock solution. The concentration of Compound 2 remaining after a period of incubation (0, 5, 10, 20, 30, and 60 minutes) was measured by LC-MS/MS. The CYP probe substrates and metabolites used to verify CYP enzyme activities is shown in TABLE 143 above. CYP reaction phenotyping was performed using hrCYPs by an in vitro intrinsic clearance approach. Compound 2 at one concentration (1 μM in the final incubation) was incubated with an individual hrCYP (20 pmol CYP/mL) or CYP control (negative control without CYP enzymes, 0.1 mg protein/mL) in phosphate buffer (100 mM, pH 7.4) containing MgCl₂ (5 mM) and NADPH (1 mM). The incubation mixture without NADPH was equilibrated in a shaking water bath at 37° C. for 5 minutes. The reaction was initiated by adding NADPH (1 mM), followed by incubation at 37° C. Aliquots (100 μL) of the incubation solutions were sampled at 0, 5, 10, 20, 30, and 60 minutes (n=3). The reaction was terminated by the addition of ice-cold acetonitrile containing an internal standard (IS, 0.2 μM metoprolol) (acetonitrile:IS 1:2, v/v). After the removal of protein by centrifugation at 1,640 g (3,000 rpm) for 10 minutes at 4° C., the supernatants were transferred to an HPLC autosampler plate and stored at −20° C. until analysis. The concentration of Compound 2 remaining (expressed as the peak area ratio of Compound 2 to IS) was determined by LC-MS/MS. hrCYP activities were verified in parallel by determining the formation of CYP probe metabolites after 20 minutes of incubation with individual CYP probe substrates by LC-MS/MS using standard curves

The percent remaining of the Compound 2 was calculated using the following equation: % Remaining of the TA=100×At/A0; wherein At is the peak area ratio of Compound 2 to IS at time t and A0 is the peak area ratio of Compound 2 to IS at time zero. The elimination rate constant of Compound 2 was estimated from first-order reaction kinetics: Cₜ=C₀·e^{-kt}; where C₀ and Cₜ are the concentrations of Compound 2 (expressed as the peak area ratios of TA to IS) at time zero and incubation time t (min), and k is the elimination rate constant (min-1). The in vitro intrinsic clearance of Compound 2 was calculated using the following equation: CLᵢₙₜ=k/P; where CLᵢₙₜ is the in vitro intrinsic clearance, k is the elimination rate constant (min-1); and P is the enzyme concentration in the incubation (pmol CYP/mL or mg Supersome protein/mL). Corrected CLᵢₙₜ (mL/min/mg Supersome protein)=CLᵢₙₜ (hrCYP)−CLᵢₙₜ (Negative Control); Scaled CLᵢₙₜ (mL/min/mg liver microsomal protein)=Corrected CLᵢₙₜ (mL/min/mg Supersome protein)×CYP abundance in HLM (pmol CYP/mg microsomal protein)×[CYP protein content (mg Supersome protein/mL)/CYP content (pmol CYP/mL)]. The percent contribution of an individual CYP enzyme to the overall oxidative metabolism was estimated by the following equation for rank-order evaluation: % Relative contribution of an individual CYP enzyme=100×[CLᵢₙₜ of an individual CYP enzyme×CYP abundance in HLM/Σ(CLᵢₙₜ×CYP abundance)]; or =100×[Scaled CLᵢₙₜ of an individual CYP enzyme/Σ(Scaled CLᵢₙₜ of all responsible CYP enzymes)].

Results: The percent remaining and intrinsic clearance of Compound 2 (1 μM) in hrCYPs (20 pmol CYP/mL) are summarized in TABLE 196. hrCYP activities were verified in parallel by determining the formation of CYP probe metabolites using LC-MS/MS, and the results are summarized in TABLE 197

TABLE 196

| | | | | | | | | | | | | Relative |
|---|---|---|---|---|---|---|---|---|---|---|---|---|
| | Vₒ Remaining of TA | | | | | | | Raw | Normalized | Corrected | Scaled | Contribution^{f} |
| Itr CYP | n = 3 | 0 Min | 5 Min | 10 Min | 20 Min | 30 Min | 60 min | CLint^{b} | CLint | cLintd | cLinte | (%) |
| CYPIA2 | Average | 100 | 96.8 | 92.2 | 95.0 | 95.3 | 88.9 | 0.0750 | 14.7 | 0 | 0 | 0 |
| | SD | 1.86 | 2.90 | 5.80 | 1.38 | 5.54 | 1.07 | | | | | |
| CYP2B6 | Average | 100 | 98.1 | 95.9 | 98.6 | 97.7 | 95.1 | 0.0292 | 3.18 | 0 | 0 | 0 |
| | SD | 3.77 | 4.17 | 3.26 | 1.18 | 1.16 | 9.19 | | | | | |
| CYP2C8 | Average | 100 | 96.4 | 93.0 | 93.5 | 92.6 | 90.3 | 0.0695 | 9.65 | 0 | 0 | 0 |
| | SD | 1.64 | 1.38 | 3.72 | 3.75 | 4.85 | 4.06 | | | | | |
| CYP2C9 | Average | 100 | 91.9 | 87.6 | 92.0 | 93.9 | 86.7 | 0.0703 | 27.0 | 4.68 | 0.742 | 1.66 |
| | SD | 3.63 | 1.39 | 3.37 | 2.35 | 2.03 | 2.00 | | | | | |
| CYP2C19 | Average | 100 | 87.1 | 81.2 | 82.2 | 74.3 | 66.2 | 0.315 | 85.2 | 62.8 | 2.56 | 5.72 |
| | SD | 3.08 | 3.59 | 1.49 | 6.26 | 3.92 | 5.33 | | | | | |
| CYP2D6 | Average | 100 | 98.0 | 97.0 | 100 | 92.8 | 89.8 | 0.0874 | 9.50 | 0 | 0 | 0 |
| | SD | 2.78 | 4.85 | 3.16 | 1.93 | 2.57 | 2.68 | | | | | |
| CYP3A4 | Average | 100 | 80.5 | 71.3 | 61.9 | 51.1 | 48.5 | 0.713 | 59.4 | 37.1 | 41.4 | 92.6 |
| | SD | 5.61 | 5.55 | 2.98 | 4.01 | 4.53 | 1.69 | | | | | |
| Cyp | Average | 100 | 93.3 | 96.3 | 98.7 | 87.3 | 86.6 | | 22.3 | 0 | | |
| Control | SD | 3.58 | 1.69 | 3.70 | 1.41 | 3.56 | 3.16 | | | | | |

^{a} The % remaining(n = 3) of the TA was calculated from the peak area ratio of the TA to IS by LC-MS/MS.
^{b}The raw intrinsic clearance (CLiₙₜ) is expressed as ttL/min/pmol CYP for nCYPs (20 pmol CTP/mL).
cThe intrinsic clearance (CLiₙₜ) was normalized and expressed as μL/min/mg Supersome protein, based on the Supersome protein and luCTP concentrations.
dCorrected CLiₙt = nomalized CLint ancm − CLint (Negative control). No clearance (with a negative value) is reported as zero.
eScaled CLiₙₜ was calculated from the individual CYP abundance in HLM, and expressed as μL/min/mg liver microsomal protein. No clearance is reported as zero.
^{f} Relative % contribution (for rank-order evaluation) = 100 × [CIA, or an individual CYP enzyme × CYP abundance in HLM/1(CLiₙₜ × CYP abundance)] = 100 × [Scaled CLiₙₜ of an individual CYP enzyme/Z(Scaled CLiₙₜ of all responsible CYP enzymes)].

TABLE 197

| | | | Measured | | Formation |
|---|---|---|---|---|---|
| Probe | | | Metabolite | Formation | Rate |
| Substrate | Metabolite | Treatment | (μM)^{a} | Rate^{b} | Ratio^{c} |
| Phenacetin | Acetaminophen | hrCYP1A2 | 3.71 | 1819 | >>2 |
| (50 μM) | | CYP Control | 0 | 0 | |
| Bupropion | OH Bupropion | hrCYP2B6 | 0.633 | 172 | >>2 |
| (50 μM) | | CYP Control | 0 | 0 | |
| Amodiaquine | Desethylamodiaquine | hrCYP2C8 | 1.42 | 491 | >>2 |
| (2 μM) | | CYP Control | 0 | 0 | |
| Diclofenac | 4′- OH Diclofenac | hrCYP2C9 | 0.676 | 650 | >>2 |
| (6 μM) | | CYP Control | 0 | 0 | |
| S-mephenytoin | 4′ - OH | hrCYP2C19 | 7.96 | 5375 | >>2 |
| (20 μM) | Mephenytoin | CYP Control | 0 | 0 | |
| Bufuralol | 1′ - OH Bufuralol | hrCYP2D6 | 2.94 | 799 | >>2 |
| (7 μM) | | CYP Control | 0 | 0 | |
| Midazolam | 1′ - OH Midazolam | hrCYP3A4 | 2.31 | 481 | >>2 |
| (2 μM) | | CYP Control | 0 | 0 | |
| Testosterone | 6β-OH Testosterone | hyCYP3A4 | 23.6 | 4917 | >>2 |
| (50 μM) | | CYP Control | 0 | 0 | |

^{a}The concentrations (average, n = 2) of CYP probe metabolites were measured by LC-MS/MS.
^{b}The formation rates of CYP probe metabolites were normalized and expressed as pmol metabolite/min/mg Supersome protein. When a probe metabolite was not detectable, the rate of formation is reported as zero.
^{c}Formation rate ratio = Formation rate (hrCYP)/Formation rate (Negative Control). QC acceptable criterion: formation rate ratio ≥2.

CYP reaction phenotyping using hrCYPs showed that Compound 2 was metabolized predominantly by CYP3A4 and slightly by CYP2C19, while CYP1A2, CYP2B6, CYP2C8, CYP2C9, and CYP2D6 were not involved in the metabolism of Compound 2 in the study. Liquid chromatography and mass spectrometry were performed as described in EXAMPLE 20. The gradient programs specific to acetaminophen, 4′-OH diclofenac, 4′-OH mephentoin, testosterone, and all other CYPs are shown in TABLES 146-150. Mass spectrometry parameter settings are shown in TABLE 151.

Calibration Curves: Calibration curves for the quantification of CYP probe metabolites were prepared by fortifying standard solutions of the metabolites into blank incubation medium at six to eight concentrations. The fortified standard solutions were treated by the addition of protein precipitation solvent. After centrifugation at 1,640 g (3,000 rpm) for 10 minutes, the supernatants were analyzed by LC-MS/MS. The acceptance criterion for the calibration curve was at least 75% of standards within 85% to 115% accuracy except at the LLOQ, where 80% to 120% accuracy was acceptable.

CYP reaction phenotyping using hrCYPs showed that Compound 2 was metabolized predominantly by CYP3A4 (˜93% relative contribution based on the scaled CLᵢₙₜ) and slightly by CYP2C19 (˜5.7% relative contribution). Although the relative contribution of CYP2C9 was mathematically calculated to be 1.66% based on the scaled CLᵢₙₜ, no difference was observed in the disappearance of Compound 2 between hrCYP2C9 and the negative control (without CYP) (86.7% vs. 86.6% remaining after 60 minutes of incubation). The results show that the involvement of CYP2C9 in the metabolism of Compound 2 was minimal. CYP1A2, CYP2B6, CYP2C8, and CYP2D6 were not involved in the metabolism of Compound 2 in the study.

### Example 30: Cytochrome P450 Reaction Phenotyping of Compound 2 Using Human Liver Microsomes and Chemical Inhibitors

Cytochrome P450 (CYP) reaction phenotyping of Compound 2 was evaluated using HLM in the absence and presence of an individual CYP-selective inhibitor by an in vitro intrinsic clearance approach. Compound 1 (1 μM) was incubated with pooled HLM (0.5 mg protein/mL) in phosphate buffer (100 mM, pH 7.4) containing MgCl₂ (5 mM) and NADPH (1 mM), in the absence and presence of a CYP-selective inhibitor. The amount of Compound 2 remaining after a period of incubation (0, 5, 10, 20, 30, and 60 minutes) was measured by LC-MS/MS.

Compound 2 was prepared in DMSO and diluted using methanol. CYP-selective inhibitors are shown in TABLE 198. All other chemicals and reagents were of analytical grade or higher. Pooled HLM were stored at −80° C. until use. CYP reaction phenotyping was performed using HLM and CYP-selective inhibitors by an in vitro intrinsic clearance approach. Compound 2 at one concentration (1 μM in the final incubation) was incubated with HLM (0.5 mg protein/mL) in phosphate buffer (100 mM, pH 7.4) containing MgCl₂ (5 mM) and NADPH (1 mM), in the absence and presence of an individual CYP-selective inhibitor. The reaction mixture without NADPH was equilibrated in a shaking water bath at 37° C. for 5 minutes. The reaction was initiated by adding NADPH, followed by incubation at 37° C. For irreversible incubation, the inhibitor was pre-incubated with HLM in the presence of NADPH at 37° C. for 15 minutes and the reaction was initiated by adding Compound 2. Aliquots of the incubated solutions were sampled at 0, 5, 10, 20, 30, and 60 minutes. The reaction was terminated by adding ice-cold acetonitrile containing an internal standard (IS, 0.2 μM metoprolol). After the removal of protein by centrifugation at 1,640 g (3,000 rpm) for 10 minutes at 4° C., the supernatants were transferred to an HPLC autosampler plate. The concentration of the Compound 2 remaining was determined by LC-MS/MS. CYP enzyme activities of the HLM were verified in parallel by determining the formation of individual CYP probe metabolites by LC-MS/MS using standard curves.

TABLE 198

| | | Concentration | Pre- | Inhibition |
|---|---|---|---|---|
| CYP | Inhibitor | (μM) | incubation | Condition |
| CYP1A2 | Furafylline | 10 | Yes | Irreversible |
| CYP2B6 | Thio-TEPA | 10 | No | Reversible |
| CYP2C8 | Montelukast | 5 | No | Reversible |
| CYP2C9 | Sulfaphenazole | 5 | No | Reversible |
| CYP2C19 | (+)-N-3-benzylnirvanol | 5 | No | Reversible |
| | (N3B) | | | |
| CYP2D6 | Quinidine | 1 | No | Reversible |
| CYP3A | Ketoconazole | 1 | No | Reversible |
| Negative Control −1 | -inhibitor, −NADPH | N/A | Yes | Irreversible |
| Negative Control −2 | -inhibitor, −NADPH | N/A | No | Reversible |
| Maximum Metabolism-1 | -inhibitor, +NADPH | N/A | Yes | Irreversible |
| Maximum Metabolism-2 | -inhibitor, +NADPH | N/A | No | Reversible |

The percent remaining of Compound 2 and elimination rate constant of Compound 2 were calculated as described in EXAMPLE 29. The in vitro intrinsic clearance of the TA was calculated using the following equation: CLᵢₙₜ=k/P; where, CLᵢₙₜ is the in vitro intrinsic clearance, k is the elimination rate constant (min-1), and P is the protein concentration of HLM (mg/mL) in the incubation. Corrected CLᵢₙₜ=Raw CLᵢₙₜ−Negative Control CLᵢₙₜ; Δ CLᵢₙₜ=CLHᵢₙₜ(TA+NADPH−Inhibitor)−CLᵢₙₜ (TA+NADPH+Inhibitor). The percent remaining and intrinsic clearance (CLᵢₙₜ) of Compound 2 (1 μM) with pooled HLM (0.5 mg protein/mL) in the absence and presence of an individual CYP-selective inhibitor are shown in TABLE 199 and TABLE 200. CYP enzyme activities of the HLM were verified in parallel by determining the formation of CYP probe metabolites using LC-MS/MS, and the results are shown in TABLE 201.

TABLE 199
Intrinsic clearance of Compound 2 in HLM (Irreversible incubation conditions)

| | | % Remaining of TA^{a} | | | | | | | Raw | Corrected | A |
|---|---|---|---|---|---|---|---|---|---|---|---|
| Reaction | Treatment | N = 3 | 0 Min | 5 Min | 10 Min | 20 Min | 30 Min | 60 min | Clint^{b} | CLint^{c} | CLillt^{d} |
| Negative | −NADPH | Ave | 100 | 106 | 111 | 109 | 108 | 108 | 0 | 0 | — |
| Control-1 | −Inhibitor | SD | 3.01 | 2.09 | 4.58 | 2.60 | 3.99 | 1.77 | | | |
| Maximum | +NADPH | Ave | 100 | 93.6 | 91.1 | 77.2 | 63.0 | 37.1 | 31.9 | 31.9 | 0 |
| Metabolism-1 | −Inhibitor | SD | 6.67 | 1.93 | 2.89 | 4.50 | 1.79 | 1.76 | | | |
| CYP1A2 | +NADPH | Ave | 100 | 110 | 113 | 96.9 | 90.8 | 59.2 | 17.7 | 17.7 | 14.2 |
| | +Inhibitor | SD | 10.4 | 5.93 | 5.27 | 7.23 | 6.91 | 7.54 | | | |

^{a}The % remaining (n = 3) was calculated from the peak area ratio of the TA to IS by LC-MS/MS.
^{b}CLint is expressed as μL/min/mg protein
^{c}Corrected CLint (μL/minting protein) = Raw CLint − Negative Control CLint.
^{d}A CLiₙₜ (μL/min/mg protein, for rank order evaluation) = CLint (TA + NADPH − Inhibitor) − CLint (TA + NADPH + Inhibitor).

TABLE 200
Intrinsic clearance of Compound 2 in HLM (Reversible incubation conditions)

| | | % Remaining of Compound 2^{a} | | | | | | | Raw | Corrected | A |
|---|---|---|---|---|---|---|---|---|---|---|---|
| Reaction | Treatment | n = 3 | 0 Min | 5 Min | 10 Min | 20 Min | 30 Min | 60 min | Clint^{b} | CLint^{c} | CLillt^{d} |
| Negative | −NADPH | Ave | 100 | 101 | 99.6 | 99.9 | 99.5 | 104 | 0 | 0 | — |
| Control-2 | −Inhibitor | SD | 4.21 | 3.50 | 1.58 | 2.29 | 2.15 | 5.63 | | | |
| Maximum | +NADPH | Ave | 100 | 102 | 93.7 | 841 | 75.5 | 52.4 | 22.0 | 22.0 | 0 |
| Metabolism-2 | −Inhibitor | SD | 4.55 | 4.03 | 3.39 | 2.31 | 4.05 | 1.62 | | | |
| CYP2B6 | +NADPH | Ave | 100 | 89.3 | 79.9 | 72.7 | 60.7 | 38.5 | 31.5 | 31.5 | 0 |
| | −Thio-TEPA | SD | 5.76 | 4.22 | 2.81 | 4.89 | 2.71 | 3.97 | | | |
| CYP2C8 | +NADPH | Ave | 100 | 86.0 | 88.7 | 74.8 | 66.7 | 47.2 | 24.6 | 24.6 | 0 |
| | −Montelukast | SD | 5.20 | 2.24 | 2.87 | 0.520 | 2.81 | 2.02 | | | |
| CYP2C9 | +NADPH | Ave | 100 | 93.3 | 93.6 | 79.4 | 69.0 | 47.5 | 24.8 | 24.8 | 0 |
| | Sulfaphenazole | SD | 3.44 | 1.40 | 1.60 | 4.19 | 1.43 | 1.95 | | | |
| CYP2C19 | +NADPH | Ave | 100 | 87.0 | 85.6 | 71.6 | 63.2 | 38.3 | 30.8 | 30.8 | 0 |
| | N3B | SD | 5.11 | 3.01 | 0.402 | 3.48 | 0.186 | 1.65 | | | |
| CYP2D6 | +NADPH | Ave | 100 | 95.7 | 86.5 | 77.1 | 62.7 | 39.4 | 31.1 | 31.1 | 0 |
| | Quinidine | SD | 4.10 | 6.68 | 0.988 | 2.89 | 1.36 | 2.17 | | | |
| CYP3A | +NADPH | Ave | 100 | 97.4 | 98.4 | 91.6 | 83.7 | 65.3 | 14.0 | 14.0 | 7.97 |
| | Ketoconazole | SD | 6.28 | 2.57 | 0.167 | 2.95 | 3.09 | 1.69 | | | |

^{a}The % remaining (n = 3) was calculated from the peak area ratio of the TA to IS by LC-MS/MS.
^{b}CLint is expressed as μL/min/mg protein
^{c}Corrected CLint (−IL/minting protein) = Raw CLint − Negative Control CLint.
^{d}A CLiₙₜ (IL/min/mg protein, for rank order-evaluation) = CLint (TA + NADPH − Inhibitor) − CLint (TA
+ NADPH + Inhibitor).

TABLE 201
Formation of CYP Probe Metabolites in HLM

| | | | | Measured | | Formation |
|---|---|---|---|---|---|---|
| | | | | Metabolite | Formation | Rate |
| Probe | Metabolite | Treatment | Reaction | (μM)^{a} | Rate^{b} | Ratio^{c} |
| Phenacetin | Acetaminophen | −NADPH | Negative | 0 | 0 | — |
| (25 μM) | | −Inhibitor | Control | | | |
| | | +NADPH | Maximum | 2.21 | 221 | 47.3 |
| | | −Inhibitor | Metabolism | | | |
| | | +NADPH | CYP1A2 | 0.0466 | 4.66 | |
| | | +Furafylline | | | | |
| Bupropion | OH | −NADPH | Negative | 0 | 0 | — |
| (100 μM) | Bupropion | −Inhibitor | Control | | | |
| | | +NADPH | Maximum | 0.908 | 90.8 | 2.32 |
| | | −Inhibitor | Metabolism | | | |
| | | +NADPH | CYP2B6 | 0.391 | 39.1 | |
| | | +Thio-TEPA | | | | |
| Amodiaquine | Desethylamodiaquine | −NADPH | Negative | 0.00950 | 0.950 | — |
| (1 μM) | | −Inhibitor | Control | | | |
| | | +NADPH | Maximum | 0.651 | 65.1 | 4.51 |
| | | −Inhibitor | Metabolism | | | |
| | | +NADPH | CYP2C8 | 0.152 | 15.2 | |
| | | +Montelukast | | | | |
| Diclofenac | 4′-OH | −NADPH | Negative | 0 | 0 | — |
| (4 μM) | Diclofenac | −Inhibitor | Control | | | |
| | | +NADPH | Maximum | 4.71 | 471 | 7.37 |
| | | −Inhibitor | Metabolism | | | |
| | | +NADPH | CYP2C9 | 0.638 | 63.8 | |
| | | +Sulfaphenazole | | | | |
| S-Mephenytoin | 4′-OH | −NADPH | Negative | 0 | 0 | — |
| (25 μM) | Mephenytoin | −Inhibitor | Control | | | |
| | | +NADPH | Maximum | 0.398 | 39.8 | 5.19 |
| | | −Inhibitor | Metabolism | | | |
| | | +NADPH | CYP2C19 | 0.0766 | 7.66 | |
| | | +N3B | | | | |
| Bufuralol | 1′-OH | −NADPH | Negative | | | — |
| (7 μM) | Bufuralol | −Inhibitor | Control | | | |
| | | +NADPH | Maximum | | | 5.39 |
| | | −Inhibitor | Metabolism | | | |
| | | +NADPH | CYP2D6 | | | |
| | | +Quinidine | | | | |
| Midazolam | 1′-OH | −NADPH | Negative | | | — |
| (2 μM) | Bufuralol | −Inhibitor | Control | | | |
| | | +NADPH | Maximum | | | 9.71 |
| | | −Inhibitor | Metabolism | | | |
| | | +NADPH | CYP3A | | | |
| | | +Ketoconazole | | | | |
| Testosterone | 6β-OH | −NADPH | Negative | | | — |
| (70 μM) | Testosterone | −Inhibitor | Control | | | |
| | | +NADPH | Maximum | | | 23.5 |
| | | −Inhibitor | Metabolism | | | |
| | | +NADPH | CYP3A | | | |
| | | +Ketoconazole | | | | |

^{a}The concentrations (average, n = 2) of CYP probe metabolites were measured by LC-MS/MS using standard curves.
^{b}The formation rates of CYP probe metabolites are expressed as pmol/min/mg protein. When a probe metabolite was not detectable, the rate of formation was reported as zero.
^{c}Formation rate ratio = [Formation Rate_{(+NADPH-Inhibitor)} − Formation Rate_{(-NADPH-Inhibitor)}]/[Formation Rate_{(+NADPH+Inhibitor)} − Formation Rate_{(-NADPH-Inhibitor)}]. QC acceptance criterion: formation ratio ≥2.

Liquid chromatography and mass spectrometry were performed as described in EXAMPLE 19 and 20. The gradient programs specific to acetaminophen, 4′-OH diclofenac, 4′-OH mephentoin, testosterone, and all other CYPs are shown in TABLES 146-150. The MS parameter settings are shown in TABLE 151. Calibration curves for quantification of CYP probe metabolites were prepared as described in EXAMPLE 29.

CYP reaction phenotyping using HLM with CYP-selective inhibitors showed that Compound 2 was metabolized in HLM and inhibited by furafylline (a selective inhibitor of CYP1A2) and ketoconazole (a selective inhibitor of CYP3A), while Thio-TEPA (a selective inhibitor of CYP2B6), montelukast (a selective inhibitor of CYP2C8), sulfaphenazole (a selective inhibitor of CYP2C9), (+)-N-3-benzylnirvanol (a selective inhibitor of CYP2C19), and quinidine (a selective inhibitor of CYP2D6) did not inhibit the metabolism of Compound 2 in HLM. The results suggest that Compound 2 was metabolized by CYP1A2 and CYP3A, while CYP2B6, CYP2C8, CYP2C9, CYP2C19, and CYP2D6 were unlikely to be responsible for the metabolism of Compound 2. Compound 2 was stable in the incubation matrices containing HLM in the absence of NADPH, suggesting that the metabolism of Compound 2 in HLM is NADPH-dependent (through CYP and/or flavin-containing monooxygenase (FMO)).

### Example 31: Determination of Metabolic Stability of Compound 2 in Human and Animal Cryopreserved Hepatocytes Followed by Metabolite Profiling in Human Hepatocytes and Detection of Human Metabolites in Animal Matrices

The metabolic stability of Compound 2 in human and animal cryopreserved hepatocytes was determined, followed by metabolite profiling in human hepatocytes and detection of human metabolites in animal matrices. Metabolism of Compound 2 was studied in human and animal cryopreserved hepatocytes at two concentrations (1 and 50 μM), followed by detection of human metabolites in animal matrices.

Biological Experimental Details: Experiments with Compound 2 and positive control compounds were run in parallel to confirm the viability of the human and animal cryopreserved hepatocytes used in the study. The incubation samples were extracted with acetonitrile containing an IS. After centrifugation, clear supernatants were transferred for analysis by LC-MS/MS for metabolic stability or by LC-HRAMS for metabolite profiling using an Orbitrap mass spectrometer.

Compound 2 was dissolved in DMSO to prepare a stock solution (10 mM), which was kept at −20° C. Mixed-gender human cryopreserved hepatocytes, male Sprague-Dawley rat cryopreserved hepatocytes, male CD-1 mouse cryopreserved hepatocytes, male beagle dog cryopreserved hepatocytes, and male cynomolgus monkey cryopreserved hepatocytes were thawed and pooled into KHB buffer (pH 7.4) and kept on ice prior to the experiments. The hepatocyte suspension was equilibrated in a shaking water bath at 37° C. for 3 minutes, and then the reaction was initiated by spiking Compound 2 into the hepatocyte suspension (1.5×10⁶ cells/mL) at final concentrations of 1 and 50 μM. Each experiment was performed in triplicate. The final DMSO content in the incubation mixture was ≤0.1%. The reaction mixture was incubated in a shaking water bath at 37° C.

Positive controls, testosterone (1 μM) and 7-hydroxycoumarin (7-HC) (100 μM), were performed in parallel to confirm the activity of the hepatocytes. Aliquots of Compound 2 were withdrawn (n=1) at 0 and 120 minutes. Aliquots of testosterone were withdrawn (n=1) at 0, 5, 15, 30, 60, and 120 minutes. Aliquots of 7-HC were withdrawn (n=1) at 0 and 15 minutes. The reaction was immediately terminated by adding three volumes of ice-cold MeCN containing IS. The samples were then mixed and spun by centrifuge to precipitate proteins. An aliquot of the supernatant was then diluted with water. Calibration standards for the analysis of 7-HC metabolites were prepared in matched matrix. Compound 2 and testosterone samples were analyzed without calibration standards. All samples were analyzed by LC-MS/MS. Analytical conditions are outlined below. The peak area response ratio (PARR) vs. IS was compared to the PARR at time 0 to determine the percent remaining at each time point. Half-lives and clearance values were calculated using GraphPad software, fitting to a single-phase exponential decay equation.

Analytical methods: LC-HRAMS was used for metabolic profiling using a Dionex XR3000 quaternary solvent HPLC system equipped with column compartment thermostat (set to +40° C.) for chromatographic separation. The column was a PFP-C18, 3.0 μm, 100×2.1 mm; mobile phase A was 0.1% AcOH in water; mobile phase B was acetonitrile; and the gradient and flow profile is shown in

TABLE 202
TABLE 202. The HRAMS LTQ Orbitrap operational parameters
and LC-MS/MS methods for determining metabolic
stability are described in EXAMPLE 28.

| Time (minutes) | % B | Flow (mL/min) |
|---|---|---|
| 0.0 | 2 | 0.30 |
| 20.0 | 15 | 0.30 |
| 38.0 | 100 | 0.30 |
| 40.0 | 100 | 0.35 |
| 40.1 | 2 | 0.40 |
| 43.0 | 2 | 0.35 |
| 470 | 2 | 0.30 |

Metabolite Profiling: The samples were analyzed using HPLC, and the column eluate was surveyed using an LTQ Orbitrap hybrid instrument. The MS instrument combines a linear ion trap (LTQ) and high resolution FT mass analyzer (Orbitrap). The survey MS scan in positive mode or negative mode (separate injections) was performed on the Orbitrap FT analyzer, which was operated at a resolution of Rs=60,000 (m/z range 150-900 Th). The cycle started with an FT pre-scan. In this pre-scan, the FT analyzer was operated at a high acquisition rate and a lower resolution (Rs=7500). The FT pre-scan was used to calculate optimal parameters for the survey's high resolution scan. The pre-scan also returned corresponding m/z values of all ions present in the HPLC eluate. Following the pre-scan, the FT analyzer was set to perform a slow survey scan at high resolution (HRAMS). In parallel, the LTQ ion trap was set to acquire MS(n) data using a data-dependent acquisition (DDA) event. The DDA consisted of the decision event and three MS2 product ion scans. The decision event selected the four most intense ions detected in the pre-scan that were on the parent mass list (m/z of molecular ions of expected metabolites), or if none were observed, the most intense ions were selected. The data was processed using Compound Discoverer software. The ratio S/N=1.5 was used for peak detection.

TABLE 203 shows the results of relative quantification of the percent remaining of Compound 2 after 120 minutes in human and animal hepatocytes.

TABLE 203

| | | Concentration | % Remaining at |
|---|---|---|---|
| Test Article | Species | (μM) | 120 Minutes |
| Compound 2 | Human | 1 | 88.7 |
| | | 50 | 109 |
| | Rat | 1 | 98.1 |
| | | 50 | 113 |
| | Mouse | 1 | 87.5 |
| | | 50 | 100 |
| | Dog | 1 | 91.9 |
| | | 50 | 97.2 |
| | Monkey | 1 | 81.5 |
| | | 50 | 117 |

Data processing of the human incubation sample with C₀=50 μM led to the detection of a total of ten peaks of putative metabolites (M1-M10). TABLE 204 shows the results of partial characterization of putative metabolites M1-M10 of Compound 2 generated in human hepatocytes and the relative levels in human and animal matrices. Abbreviations—RT: retention time; MW: molecular weight; NE: not established, mechanistic assignment of the nature of biotransformation could not be established based on HRAMS data; ND: peak was not detected.

TABLE 204

| | | | | | Relative levels | | | | |
|---|---|---|---|---|---|---|---|---|---|
| | | | | | (% to TA) in 50 μM | | | | |
| | | | | Putative | Incubation Samples | | | | |
| Analyte | RT. Min | MW, Da | Shift, DA | Biotransformation | Human | Rat | Mouse | Dog | Monkey |
| TA | 27.0 | 545.2417 | 0.0000 | None | 100 | 100 | 100 | 100 | 100 |
| M1 | 27.6 | 561.2347 | 15.9931 | +O | 1.45 | 2.84 | ND | 1.13 | 3.73 |
| M2 | 27.3 | 561.2374 | 15.9957 | +O | 0.0154 | 0.0611 | ND | 0.0145 | 0.0440 |
| M3 | 27.0 | 561.2351 | 15.9934 | +O | 0.0650 | 0.145 | 0.715 | 0.0789 | 0.181 |
| M4 | 26.2 | 531.2241 | −14.0176 | −CH2 | 0.524 | ND | ND | ND | ND |
| M5 | 26.9 | 531.2249 | −14.0167 | −CH2 | 0.0391 | ND | 0.101 | 0.119 | 0.136 |
| M6 | 25.4 | 543.2245 | −2.0172 | −2H | 1.29 | 3.78 | 3.86 | 2.08 | 3.94 |
| M7 | 28.1 | 581.2175 | 35.9759 | NE | 0.271 | 0.0922 | 0.0925 | 0.226 | 0.111 |
| M8 | 27.2 | 581.2183 | 35.9766 | NE | 0.0430 | ND | ND | 0.0389 | 0.0663 |
| M9 | 29.0 | 645.293 | 100.0513 | NE | 0.152 | ND | 0.0493 | 0.159 | 0.0759 |
| M10 | 29.0 | 589.2305 | 43.9888 | NE | 0.203 | ND | ND | 0.245 | 0.108 |

Putative metabolite M1 was detected as both mono- and di-protonated molecular ions. Based on HRAMS data, putative metabolite M1 and isobaric metabolites M2 and M3 mechanistically corresponded to the results of mono-oxygenation (+O). Putative metabolite M4 was detected as both mono- and di-protonated molecular ions. Based on HRAMS data, putative metabolite M4 and isobaric metabolite M5 mechanistically corresponded to a change in elemental composition of —CH2 (presumed demethylation). Putative metabolite M6 was detected as both mono- and di-protonated molecular ions. Based on HRAMS data, putative metabolite M6 mechanistically corresponded to the result of desaturation (−2H). Putative metabolite M7 was detected as both mono- and di-protonated molecular ions. Putative metabolite M9 was detected as a monoprotonated molecular ion. Putative metabolite M10 was detected as both mono- and di-protonated molecular ions. No matrix interferences were observed with the peaks of the targeted analytes in the human solvent control sample. The detection of the peak of the IS confirmed the integrity of the injection and validates the analytical performance.

The putative human metabolites in animal incubation samples at C₀=50 μM were detected. In the rat incubation sample, the signals in the XIC channels corresponding to the peaks of putative metabolites M4, M5, and M8-M10 were within background noise, and the peaks were assigned as not detected. The peaks of putative metabolites M1, M2, and M4 were not detected in the mouse incubation sample. The signals in the XIC channels corresponding to the peaks of putative metabolites M8 and M10 were within background noise, and the peaks were also assigned as not detected. In the dog incubation sample, the signal in the XIC channel corresponding to the peak of putative metabolite M4 was within background noise, and the peak was assigned as not detected. In the monkey incubation sample, the signal in the XIC channel corresponding to the peak of putative metabolite M4 was within background noise, and the peak was assigned as not detected.

Very limited metabolite coverage was observed in all incubation samples at C0=1 μM. The peaks of putative metabolites M1 and M2 in the human incubation sample appeared to be present near the limit of detection. Peaks of putative metabolites M3-M10 were not detected.

TABLE 205 shows results of analysis of the positive control, testosterone. TABLE 206 shows rates of formation of glucuronide and sulfate of 7-Hydroxycouarin in cryopreserved hepatocytes.

TABLE 205

| | | | Half-life |
|---|---|---|---|
| | Half- | CLᵢₙₜ | Acceptance |
| | life | (mL/min/10⁶ | Criteria |
| Species | (min) | cells) | (min) |
| Human | 4.28 | 0.108 | ≤5.0 |
| Rat | 1.14 | 0.405 | ≤5.0 |
| Mouse | 2.55 | 0.181 | ≤5.0 |
| Dog | 6.88 | 0.0672 | ≤10 |
| Monkey | 3.08 | 0.150 | ≤5.0 |

TABLE 206

| | | Formation | Acceptable |
|---|---|---|---|
| | | Rate | Range |
| | | (pmol/min/10⁶ | (pmol/min/10⁶ |
| Species | Analyte | cells) | cells) |
| Human | 7-HC-G | 316 | ≥50 |
| | 7-HC-S | 7.74 | ≥1.0 |
| Rat | 7-HC-G | 253 | ≥25 |
| | 7-HC-S | 34.0 | ≥5.0 |
| Mouse | 7-HC-G | 234 | ≥10 |
| | 7-HC-S | 6.36 | ≥1.0 |
| Dog | 7-HC-G | 301 | ≥50 |
| | 7-HC-S | 24.5 | ≥5.0 |
| Monkey | 7-HC-G | 279 | ≥ |
| | 7-HC-S | 13.3 | ≥5.0 |

### Example 32: Determination of In Vitro Blood to Plasma Ratio of Compound 2 in CD-1 Mouse, SD Rat, Beagle Dog, Cynomolgus Monkey, and Human Blood

The partitioning of Compound 2 was determined in CD-1 mouse, SD rat, beagle dog, cynomolgus monkey and human blood vs. plasma. Compound 2 at 1 μM was incubated with mouse, rat, dog, monkey and human blood at 37° C. for 1 hour in a humidified incubator with 5% CO₂. Concentrations of Compound 2 in the blood and plasma were determined using LC-MS/MS. Diclofenac and chloroquine were used as control compounds to ensure system function. Diclofenac was used as a low KB/P control; chloroquine was used as a high KB/P control; tolbutamide and labetalol were used as internal standards.

A 20 mM stock solution of Compound 2 was prepared in DMSO. The working solutions of Compound 2 in DMSO at 0.2 mM were prepared by dilution from the 20 mM stock with 20% DMSO in MeOH. All working solutions were freshly prepared on the day of experiment and disposed after use. Diclofenac (0.4 mM) was prepared by diluting a 10 mM stock solution with MeOH, and chloroquine (0.4 mM) was prepared by diluting a 10 mM stock solution with water. All working solutions were freshly prepared similarly as described for the test article working solution. The stock solutions of tolbutamide and labetalol were prepared in DMSO and stored at ca. −20° C. The stop solution was prepared by spiking the stock solutions (2 mg/mL) into acetonitrile to achieve a 200 ng/mL concentration.

Assay for determining Blood to plasma ratio: Pooled male CD-1 mouse, Sprague-Dawley rat, beagle dog and cynomolgus monkey plasma, and mixed gender human blood with EDTAK2 as anticoagulant were stored on wet ice and used the same day. The blood from each species was warmed at 37° C. for 10˜15 min before use. Each 1.99 mL of dog, monkey, and human blood was spiked with 10 μL of 0.20 mM Compound 2 working solution to achieve the final concentration of 1 μM. After mixing thoroughly, 0.6 mL of spiked blood was transferred to a 96-well plate in triplicate. Concurrently, control compounds were spiked into each species blood in the same fashion to generate the final concentration of 2.0 μM.

The time zero (T₀) samples were prepared by aliquoting 50 μL of the spiked blood mixed with 50 μL of blank plasma and 100 μL water, followed by addition of 600 μL stop solution containing internal standards. The rest of the spiked blood samples were immediately placed into a humidified incubator with 5% CO₂ at 37° C., with constant swiveling at 500 rpm for 60 min on a platform shaker. At the end of 60 min incubation, the T60-blood samples were prepared by aliquoting 50 μL of blood samples from each well, mixed with 50 μL of blank plasma and 100 μL of water, followed by 600 μL stop solution. The remaining blood samples were spun by centrifuge at 37° C. for 15 min at 2,500×g to prepare plasma. The T60-plasma samples for analysis were obtained by taking 50 μL of plasma from each well, mixed with 50 μL of blank blood and 100 μL of water, followed by addition of 600 μL stop solution. Blank plasma of mouse, rat, dog, monkey and human were obtained by centrifugation at 2,500×g for 15 min from fresh whole blood at room temperature for matrix-matching during the sample processing after the partitioning incubation. All the T0, T60-blood and T60-plasma samples were shaken vigorously at 800 rpm for 30 min followed by the centrifugation at 3220×g at 20° C. for 20 min. 100 μL aliquot of supernatant was diluted with 100 μL of water and mixed well for LC-MS/MS analysis.

Measurement of Hematocrit: The hematocrit (the volume percentage of erythrocytes in blood) was determined in triplicate for each species by centrifuging the blood samples with a HAEMATOKRIT 200 at 9440×g for 5 min. The hematocrit readings were in the range of 35˜43%.

Analysis of Compound 2 and Control Samples: Concentrations of test article Compound 2 and control compounds were determined semi-quantitatively using LC-MS/MS. The peak area ratios of analyte/internal standard were used to semi-quantitatively determine the concentrations.

Data analysis: The ratio of compound concentrations in whole blood over plasma (K_{B/P}) and % Recovery in blood were calculated by the following equations: K_{B/P}=100×([T_{60-blood}]/[T₆₀₋ₚₗₐₛₘₐ]), where [T_{60-blood}] is the peak area ratios of analyte/internal standard in whole blood sample at 60 min; [T₆₀₋ₚₗₐₛₘₐ] is the peak area ratios of analyte/internal standard in plasma sample at 60 min; [T_{0-blood}] is the peak area ratios of analyte/internal standard in whole blood sample at time zero. K_{E/P}=1+([K_{B/P}−1]/[H_{C}]), where H_{C} is the hematocrit of the whole blood used in the determination. % Recovery=100×([T_{60-blood}]/[T₀₋ₚₗₐₛₘₐ]).

Results: The results of blood to plasma partitioning of Compound 2 in CD-1 mouse, SD rat, beagle dog, cynomolgus monkey and human blood are summarized in TABLE 207. Compound 2 exhibited low partitioning into erythrocytes (K_{B/P}<1) in mouse, rat and dog blood, and very low level partitioning into erythrocytes of monkey and human blood.

TABLE 207

| | Concentration | K_{B/P} | | % Recovery |
|---|---|---|---|---|
| Species | (μM) | (Mean ± S.D.) | K_{E/P} | (Mean ± S.D. |
| CD-1 Mouse | 1 | 0.806 ± 0.007 | 0.446 | 97.7 ± 3.01 |
| SD Rat | | 0.746 ± 0.027 | 0.331 | 102 ± 2.54 |
| Beagle Dog | | 0.942 ± 0.056 | 0.838 | 102 ± 8.89 |
| Cynomolgus | | 0.577 ± 0.010 | 0.039 | 90.5 ± 1.55 |
| Human | | 0.559 ± 0.029 | ~0 | 90.9 ± 1.47 |

Blood to Plasma Partitioning of Compound 2: The experimental K_{B/P}, K_{E/P} and % recovery values for Compound 2 in five species of blood are shown in TABLE 208. The K_{B/P} values of Compound 2 at concentrations of 1 μM were 0.806, 0.746, 0.942, 0.577 and 0.559 in mouse, rat, dog, monkey and human blood, respectively. The corresponding mean of K_{E/P} values were 0.446, 0.331, 0.838, 0.039 and ˜0, respectively. The mean % recovery of Compound 2 was in the acceptable range of 90.5% to 102% in blood from 5 species.

TABLE 208

| | | Analyte Peak | | | | | | |
|---|---|---|---|---|---|---|---|---|
| | Conc. | Area/IS Peak Area | | | | K_{B/P} | | % Recovery |
| Species | (μM) | T0 | T_{60-blood} | T₆₀₋ₚₗₐₛₘₐ | K_{B/P} | Mean ± S.D. | K_{E/P} | Mean ± S.D. |
| CD-1 Mouse | 1 | 0.169 | 0.158 | 0.197 | 0.801 | 0.806 ± 0.007 | 0.446 | 97.7 ± 3.01 |
| | | 0.165 | 0.164 | 0.205 | 0.803 | | | |
| | | 0.171 | 0.164 | 0.201 | 0.814 | | | |
| SD Rat | | 0.163 | 0.162 | 0.225 | 0.722 | 0.746 ± 0.027 | 0.331 | 102 ± 2.54 |
| | | 0.157 | 0.164 | 0.222 | 0.739 | | | |
| | | 0.168 | 0.168 | 0.222 | 0.775 | | | |
| Beagle Dog | | 0.153 | 0.150 | 0.168 | 0.896 | 0.942 ± 0.056 | 0.039 | 102 ± 8.89 |
| | | 0.147 | 0.165 | 0.164 | 1.004 | | | |
| | | 0.162 | 0.146 | 0.158 | 0.925 | | | |
| Cynomolgus | | 0.155 | 0.142 | 0.251 | 0.568 | 0.577 ± 0.010 | 0.039 | 90.5 ± 1.55 |
| Monkey | | 0.156 | 0.141 | 0.245 | 0.576 | | | |
| | | 0.162 | 0.144 | 0.246 | 0.588 | | | |
| Human | | 0.165 | 0.147 | 0.268 | 0.550 | 0.559 ± 0.029 | ~0 | 90.9 ± 1.47 |
| | | 0.156 | 0.144 | 0.270 | 0.535 | | | |
| | | 0.173 | 0.157 | 0.266 | 0.591 | | | |

T₀: Whole blood sample taken at time zero, value represent the ratio of analyst area to IS area
T_{60-blood}: Whole blood sample taken at 60 min. value represent the ratio of analyst area to IS area
T₆₀₋ₚₗₐₛₘₐ: Plasma sample taken at 60 min. value represent the ratio of analyst area to IS area
K_{B/P}: Ratio of blood over plasma concentration
K_{E/P}: Ratio of erythrocytes over plasma concentration
SD: Standard deviation

Blood to Plasma Partitioning of Positive Controls: The experimental K_{B/P}, K_{E/P} and % recovery values for control compounds in mouse, rat, dog, monkey, and human blood are shown in TABLE 209. The data of diclofenac, a marker compound with low blood cell partitioning, and chloroquine, a control compound with high blood cell partitioning, values were within the acceptance criteria, indicating that the test systems were fully functional. Compound 2 exhibited low partitioning into erythrocytes (K_{B/P}<1) in mouse, rat and dog, and very low level in erythrocytes of monkey and human. TABLE 210 shows mean % hematocrit readings of CD-1 mouse, SD rat, beagle dog, monkey, and human samples.

TABLE 209

| | | Analyte Peak/ | | | | | |
|---|---|---|---|---|---|---|---|
| | | IS Peak Ares (Mean) | | | K_{B/P} | | % Recovery |
| Compound ID | Species | T0 | T_{60-blood} | T₆₀₋ₚₗₐₛₘₐ | Mean ± S.D. | K_{E/P} | Mean ± S.D. |
| Diclofenac | CD-1 Mouse | 0.363 | 0.365 | 0.501 | 0.728 ± 0.022 | 0.223 | 100 ± 6.79 |
| (2 μM) | SD Rat | 0.330 | 0.331 | 0.480 | 0.689 ± 0.007 | 0.182 | 100 ± 3.45 |
| | Beagle Dog | 0.301 | 0.313 | 0.484 | 0.654 ± 0.012 | 0.040 | 104 ± 1.36 |
| | Cynomolgus | 0.314 | 0.318 | 0.513 | 0.629 ± 0.039 | 0.157 | 101 ± 8.23 |
| | Monkey | 0.341 | 0.311 | 0.473 | 0.659 ± 0.039 | 0.148 | 91.2 ± 4.43 |
| | Human | | | | | | |
| Chloroquine | CD-1 Mouse | 0.402 | 0.399 | 0.148 | 2.69 ± 2.69 | 5.83 | 99.4 ± 1.70 |
| (2 μM) | SD Rat | 0.432 | 0.432 | 0.126 | 3.44 ± 0.062 | 7.43 | 100 ± 1.86 |
| | Beagle Dog | 0.381 | 0.429 | 0.115 | 3.83 ± 0.718 | 8.86 | 113 ± 3.57 |
| | Cynomolgus | 0.424 | 0.411 | 0.134 | 3.13 ± 0.535 | 5.85 | 96.9 ± 1.83 |
| | Monkey | 0.441 | 0.399 | 0.130 | 3.07 ± 0.112 | 6.18 | 90.8 ± 6.07 |
| | Human | | | | | | |

T₀: Whole blood sample taken at time zero, value represent the ratio of analyst area to IS area
T_{60-blood}: Whole blood sample taken at 60 min. value represent the ratio of analyst area to IS area
T₆₀₋ₚₗₐₛₘₐ: Plasma sample taken at 60 min. value represent the ratio of analyst area to IS area
K_{B/P}: Ratio of blood over plasma concentration
K_{E/P}: Ratio of erythrocytes over plasma concentration
SD: Standard deviation

TABLE 210

% Hematocrit Reading

| Species | Mean | S.D. |
|---|---|---|
| CD-1 Mouse | 35 | 0.58 |
| SD Rat | 38 | 0.00 |
| Beagle Dog | 36 | 0.58 |
| Monkey | 44 | 0.00 |
| Human | 41 | 2.31 |

The following non-limiting embodiments provide illustrative examples of the invention, but do not limit the scope of the invention.

Embodiment 1. A method of treating a cancer, the method comprising administering to a subject in need thereof a therapeutically-effective amount of a compound that binds to a mutant p53 protein and reconforms the mutant p53 protein to a conformation of p53 that exhibits anti-cancer activity, wherein the mutant p53 protein comprises a mutation at Y220C, wherein the compound has a half-maximal inhibitory concentration (IC₅₀) in a cancer cell that has a Y220C mutant p53 protein that is at least about 2-fold lesser than in a cancer cell that does not have any Y220C mutant p53 protein.

Embodiment 2. The method of embodiment 1, wherein the therapeutically-effective amount is from about 500 mg to about 2000 mg.

Embodiment 3. The method of embodiment 1 or 2, wherein the therapeutically-effective amount is about 600 mg.

Embodiment 4. The method of any one of embodiments 1-3, wherein the therapeutically-effective amount is about 1200 mg.

Embodiment 5. The method of any one of embodiments 1-4, wherein the compound selectively binds the mutant p53 protein compared to a wild type p53 protein.

Embodiment 6. The method of any one of embodiments 1-5, wherein the conformation of p53 that exhibits anti-cancer activity is a wild type conformation p53 protein.

Embodiment 7. The method of any one of embodiments 1-6, wherein the IC₅₀ of the compound is less than about 10 μM.

Embodiment 8. The method of embodiment 7, wherein the IC₅₀ of the compound is less than about 5 μM.

Embodiment 9. The method of embodiment 7 or 8, wherein the IC₅₀ of the compound is less than about 1 μM.

Embodiment 10. The method of any one of embodiments 7-9, wherein the IC₅₀ of the compound is less than about 0.5 μM.

Embodiment 11. The method of any one of embodiments 1-10, wherein the IC₅₀ of the compound is determined using an 3-(4,5-Dimethylthiazol-2-yl)-2,5 diphenyltetrazolium bromide (MTT) assay.

Embodiment 12. The method of any one of embodiments 1-11, wherein the cancer is ovarian cancer.

Embodiment 13. The method of any one of embodiments 1-11, wherein the cancer is breast cancer.

Embodiment 14. The method of any one of embodiments 1-11, wherein the cancer is lung cancer.

Embodiment 15. The method of any one of embodiments 1-14, wherein the administering is oral.

Embodiment 16. The method of any one of embodiments 1-14, wherein the administering is subcutaneous.

Embodiment 17. The method of any one of embodiments 1-16, wherein the subject is human.

Embodiment 18. The method of any one of embodiments 1-17, further comprising administering a therapeutically-effective amount of a therapeutic agent.

Embodiment 19. The method of embodiment 18, wherein the therapeutic agent is an immune checkpoint inhibitor.

Embodiment 20. The method of embodiment 19, wherein the immune checkpoint inhibitor is an anti-PD-1 agent.

Embodiment 21. The method of embodiment 20, wherein the anti-PD-1 agent is nivolumab.

Embodiment 22. The method of embodiment 20, wherein the anti-PD-1 agent is pembrolizumab.

Embodiment 23. The method of embodiment 20, wherein the anti-PD-1 agent is cemiplimab.

Embodiment 24. The method of embodiment 19, wherein the immune checkpoint inhibitor is an anti-PD-L1 agent.

Embodiment 25. The method of embodiment 24, wherein the anti-PD-L1 agent is atezolizumab.

Embodiment 26. The method of embodiment 24, wherein the anti-PD-L1 agent is avelumab.

Embodiment 27. The method of embodiment 24, wherein the anti-PD-L1 agent is durvalumab.

Embodiment 28. The method of any one of embodiments 1-27, wherein the compound is of the formula:

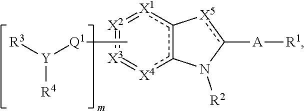

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

Embodiment 29. The method of embodiment 28, wherein A is alkylene, alkenylene, or alkynylene, each of which is substituted or unsubstituted.

Embodiment 30. The method of embodiment 28, wherein A is aryl, heteroaryl, or heterocyclyl, each of which is substituted or unsubstituted.

Embodiment 31. The method of embodiment 28 or 29, wherein the compound is of the formula:

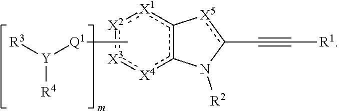

Embodiment 32. The method of embodiment 31, wherein Q¹ is C₁-alkylene.

Embodiment 33. The method of embodiment 31, wherein Q¹ is a bond.

Embodiment 34. The method of any one of embodiments 31-33, wherein m is 1.

Embodiment 35. The method of any one of embodiments 31-33, wherein m is 2.

Embodiment 36. The method of any one of embodiments 31-35, wherein Y is N.

Embodiment 37. The method of any one of embodiments 31-35, wherein Y is O.

Embodiment 38. The method of any one of embodiments 31-37, wherein each R³ and R⁴ is independently alkyl, alkylene, alkenyl, alkenylene, alkynyl, aryl, heteroaryl, or heterocyclyl, each of which is independently substituted or unsubstituted; or hydrogen.

Embodiment 39. The method of embodiment 38, wherein R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted.

Embodiment 40. The method of embodiment 38, wherein R³ is hydrogen; and R⁴ is aryl, heteroaryl, or heterocyclyl, each of which is independently substituted or unsubstituted.

Embodiment 41. The method of any one of embodiments 31-40, wherein R¹³ is hydrogen.

Embodiment 42. The method of any one of embodiments 31, 33, 34, 36 and 38-41, wherein the compound is of the formula:

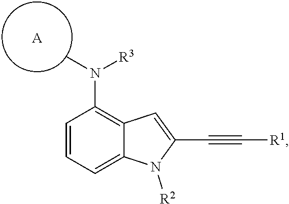

wherein ring A is a cyclic group that is substituted or unsubstituted.

Embodiment 43. The method of embodiment 42, wherein R² is substituted or unsubstituted alkyl.

Embodiment 44. The method of embodiment 42 or 43, wherein R² is methyl, ethyl, propyl, iso-propyl, butyl, or tert-butyl, each of which is substituted or unsubstituted.

Embodiment 45. The method of any one of embodiments 42-44, wherein R² is substituted ethyl.

Embodiment 46. The method of embodiment 45, wherein R² is trifluoroethyl.

Embodiment 47. The method of any one of embodiments 42-46, wherein the compound is of the formula:

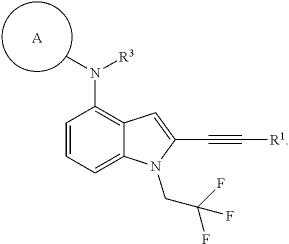

Embodiment 48. The method of embodiment 47, wherein ring A is aryl, heteroaryl, or heterocyclyl, each of which is substituted or unsubstituted.

Embodiment 49. The method of embodiment 47 or 48, wherein ring A is substituted aryl.

Embodiment 50. The method of embodiment 47 or 48, wherein ring A is substituted heteroaryl.

Embodiment 51. The method of embodiment 47 or 48, wherein ring A is substituted heterocyclyl.

Embodiment 52. The method of any one of embodiments 47-51, wherein R¹ is alkyl, alkenyl, —C(O)R¹⁶, —C(O)OR¹⁶, or —C(O)NR¹⁶R¹⁷, each of which is unsubstituted or substituted.

Embodiment 53. The method of embodiment 52, wherein R¹ is substituted alkyl.

Embodiment 54. The method of embodiment 52 or 53, wherein R¹ is alkyl substituted with NR¹⁶R¹⁷.

Embodiment 55. The method of embodiment 54, wherein the compound is of the formula:

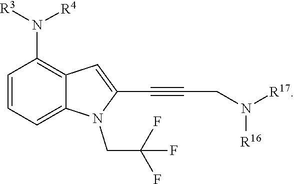

Embodiment 56. The method of embodiment 54 or 55, wherein each R¹⁶ and R¹⁷ is independently alkyl, alkenyl, aryl, heteroaryl, heterocyclyl, each of which is independently substituted or unsubstituted; or hydrogen.

Embodiment 57. The method of any one of embodiments 54-56, wherein R¹⁶ is hydrogen or alkyl.

Embodiment 58. The method of any one of embodiments 54-56, wherein R¹⁷ is aryl, heteroaryl, or heterocyclyl, each of which is independently substituted or unsubstituted.

Embodiment 59. The method of embodiment 58, wherein R¹⁷ is substituted aryl.

Embodiment 60. The method of embodiment 58 or 59, wherein R¹⁷ is substituted phenyl.

Embodiment 61. The method of any one of embodiments 58-60, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.

Embodiment 62. The method of any one of embodiments 58-61, wherein R¹⁷ is phenyl substituted with methoxy.

Embodiment 63. The method of any one of embodiments 58-61, wherein R¹⁷ is phenyl substituted with a substituted sulfoxide group.

Embodiment 64. The method of any one of embodiments 58-61, wherein R¹⁷ is phenyl substituted with a carboxyl group.

Embodiment 65. The method of any one of embodiments 58-61, wherein R¹⁷ is phenyl substituted with an amide group.

Embodiment 66. The method of embodiment 28, wherein the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide.

Embodiment 67. The method of embodiment 28, wherein the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine.

Embodiment 68. The method of embodiment 28, wherein the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide.

Embodiment 69. The method of embodiment 28, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide.

Embodiment 70. The method of embodiment 28, wherein the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide.

Embodiment 71. The method of embodiment 28, wherein the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide.

Embodiment 72. The method of embodiment 28, wherein the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide.

Embodiment 73. The method of embodiment 28, wherein the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide.

Embodiment 74. The method of embodiment 28, wherein the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

Embodiment 75. A method of treating cancer, the method comprising administering to a human in need thereof a therapeutically-effective amount of a compound, wherein the compound binds to a mutant p53 protein and reconforms the mutant p53 protein to a conformation of p53 that exhibits anti-cancer activity, wherein if in a controlled study, the therapeutically-effective amount of the compound is administered to a first subject with a cancer that expresses mutant p53, then a plasma concentration in the first subject of a protein that is a biomarker of wild-type p53 activity when measured from about 8 to about 72 hours after administration of the compound is determined to be at least about 2-fold greater than that determined in a second subject who was not administered the compound, as determined by an enzyme-linked immunosorbent assay.

Embodiment 76. The method of embodiment 75, wherein the plasma concentration in the first subject is measured about 8 hours after administration of the compound.

Embodiment 77. The method of embodiment 75, wherein the plasma concentration in the first subject is measured about 12 hours after administration of the compound.

Embodiment 78. The method of embodiment 75, wherein the plasma concentration in the first subject is measured about 24 hours after administration of the compound.

Embodiment 79. The method of any one of embodiments 75-78, wherein the biomarker of wild-type p53 activity is MDM2.

Embodiment 80. The method of any one of embodiments 75-78, wherein the biomarker of wild-type p53 activity is p21.

Embodiment 81. The method of any one of embodiments 75-80, wherein the plasma concentration of the first subject is at least about 5-fold greater than that determined in the second subject.

Embodiment 82. The method of any one of embodiments 75-80, wherein the plasma concentration of the first subject is at least about 8-fold greater than that determined in the second subject.

Embodiment 83. The method of any one of embodiments 75-80, wherein the plasma concentration of the first subject is at least about 20-fold greater than that determined in the second subject.

Embodiment 84. The method of any one of embodiments 75-80, wherein the plasma concentration of the first subject is at least about 40-fold greater than that determined in the second subject.

Embodiment 85. The method of any one of embodiments 75-84, wherein the compound is of the formula:

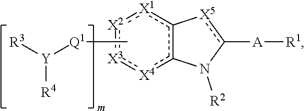

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

Embodiment 86. The method of embodiment 85, wherein A is alkylene, alkenylene, or alkynylene, each of which is substituted or unsubstituted.

Embodiment 87. The method of embodiment 85, wherein A is aryl, heteroaryl, or heterocyclyl, each of which is substituted or unsubstituted.

Embodiment 88. The method of embodiment 85 or 86, wherein the compound is of the formula:

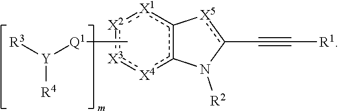

Embodiment 89. The method of embodiment 88, wherein Q¹ is C₁-alkylene.

Embodiment 90. The method of embodiment 88, wherein Q¹ is a bond.

Embodiment 91. The method of any one of embodiments 88-90, wherein m is 1.

Embodiment 92. The method of any one of embodiments 88-90, wherein m is 2.

Embodiment 93. The method of any one of embodiments 88-92, wherein Y is N.

Embodiment 94. The method of any one of embodiments 88-92, wherein Y is O.

Embodiment 95. The method of any one of embodiments 88-94, wherein each R³ and R⁴ is independently alkyl, alkylene, alkenyl, alkenylene, alkynyl, aryl, heteroaryl, or heterocyclyl, each of which is independently substituted or unsubstituted; or hydrogen.

Embodiment 96. The method of embodiment 95, wherein R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted.

Embodiment 97. The method of embodiment 95, wherein R³ is H; and R⁴ is aryl, heteroaryl, or heterocyclyl, each of which is independently substituted or unsubstituted.

Embodiment 98. The method of any one of embodiments 88-97, wherein R¹³ is hydrogen.

Embodiment 99. The method of any one of embodiments 88, 90, 91, 93 and 95-98, wherein the compound is of the formula:

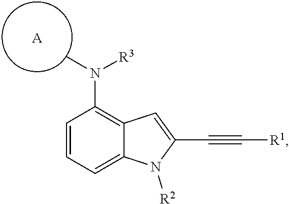

wherein ring A is a cyclic group that is substituted or unsubstituted.

Embodiment 100. The method of embodiment 99, wherein R² is substituted or unsubstituted alkyl.

Embodiment 101. The method of embodiment 99 or 100, wherein R² is methyl, ethyl, propyl, iso-propyl, butyl, or tert-butyl, each of which is substituted or unsubstituted.

Embodiment 102. The method of any one of embodiments 99-101, wherein R² is substituted ethyl.

Embodiment 103. The method of embodiment 102, wherein R² is trifluoroethyl.

Embodiment 104. The method of any one of embodiments 99-103, wherein the compound is of the formula:

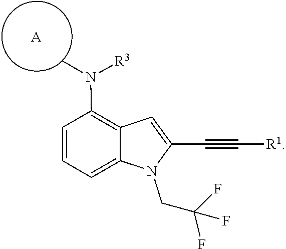

Embodiment 105. The method of embodiment 104, wherein ring A is aryl, heteroaryl, or heterocyclyl, each of which is substituted or unsubstituted.

Embodiment 106. The method of embodiment 104 or 105, wherein ring A is substituted aryl.

Embodiment 107. The method of embodiment 104 or 105, wherein ring A is substituted heteroaryl.

Embodiment 108. The method of embodiment 104 or 105, wherein ring A is substituted heterocyclyl.

Embodiment 109. The method of any one of embodiments 104-108, wherein R¹ is alkyl, alkenyl, —C(O)R¹⁶, —C(O)OR¹⁶, or —C(O)NR¹⁶R¹⁷, each of which is unsubstituted or substituted.

Embodiment 110. The method of embodiment 109, wherein R¹ is substituted alkyl.

Embodiment 111. The method of embodiment 109 or 110, wherein R¹ is alkyl substituted with NR¹⁶R¹⁷.

Embodiment 112. The method of embodiment 111, wherein the compound is of the formula:

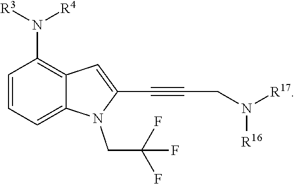

Embodiment 113. The method of embodiment 111 or 112, wherein each R¹⁶ and R¹⁷ is independently alkyl, alkenyl, aryl, heteroaryl, heterocyclyl, each of which is independently substituted or unsubstituted; or hydrogen.

Embodiment 114. The method of any one of embodiments 111-113, wherein R¹⁶ is hydrogen or alkyl.

Embodiment 115. The method of any one of embodiments 111-113, wherein R¹⁷ is aryl, heteroaryl, or heterocyclyl, each of which is independently substituted or unsubstituted.

Embodiment 116. The method of embodiment 115, wherein R¹⁷ is substituted aryl.

Embodiment 117. The method of embodiment 115 or 116, wherein R¹⁷ is substituted phenyl.

Embodiment 118. The method of any one of embodiments 115-117, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.

Embodiment 119. The method any one of embodiments 115-118, wherein R¹⁷ is phenyl substituted with methoxy.

Embodiment 120. The method of any one of embodiments 115-118, wherein R¹⁷ is phenyl substituted with a substituted sulfoxide group.

Embodiment 121. The method of any one of embodiments 115-118, wherein R¹⁷ is phenyl substituted with a carboxyl group.

Embodiment 122. The method of any one of embodiments 115-118, wherein R¹⁷ is phenyl substituted with an amide group.

Embodiment 123. The method of embodiment 85, wherein the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide.

Embodiment 124. The method of embodiment 85, wherein the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine.

Embodiment 125. The method of embodiment 85, wherein the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide.

Embodiment 126. The method of embodiment 85, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide.

Embodiment 127. The method of embodiment 85, wherein the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide.

Embodiment 128. The method of embodiment 85, wherein the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide.

Embodiment 129. The method of embodiment 85, wherein the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide.

Embodiment 130. The method of embodiment 85, wherein the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide.

Embodiment 131. The method of embodiment 85, wherein the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

Embodiment 132. A method of treating cancer, the method comprising: (i) withdrawing a first blood sample from a subject with a cancer that expresses mutant p53; (ii) measuring a first plasma concentration of a protein that is a biomarker of wild-type p53 activity in the first blood sample; (iii) after measuring the first plasma concentration of the protein that is the biomarker of wild-type p53 activity in the first blood sample, administering to the subject a therapeutically-effective amount of a compound that binds to a mutant p53 protein and reconforms the mutant p53 protein to a conformation of p53 that exhibits anti-cancer activity; (iv) withdrawing a second blood sample from the subject after administering the compound; and (v) measuring a second plasma concentration of the protein that is a biomarker of wild-type p53 activity in the second blood sample.

Embodiment 133. The method of embodiment 132, wherein the mutant p53 comprises a mutation at Y220C.

Embodiment 134. The method of embodiment 132 or 133, wherein the biomarker of wild-type p53 activity is MDM2.

Embodiment 135. The method of embodiment 132 or 133, wherein the biomarker of wild-type p53 activity is p21.

Embodiment 136. The method of any one of embodiments 132-135, further comprising determining a difference in the second plasma concentration of the protein and the first plasma concentration of the protein.

Embodiment 137. The method of embodiment 136, wherein the second plasma concentration of the protein is higher than the first plasma concentration of the protein.

Embodiment 138. The method of embodiment 136 or 137, wherein the second plasma concentration of the protein is at least about 5-fold higher than the first plasma concentration of the protein.

Embodiment 139. The method of embodiment 136 or 137, wherein the second plasma concentration of the protein is at least about 8-fold higher than the first plasma concentration of the protein.

Embodiment 140. The method of embodiment 136 or 137, wherein the second plasma concentration of the protein is at least about 20-fold higher than the first plasma concentration of the protein.

Embodiment 141. The method of embodiment 136 or 137, wherein the second plasma concentration of the protein is at least about 40-fold higher than the first plasma concentration of the protein.

Embodiment 142. The method of embodiment 136, wherein the second plasma concentration of the protein is equal to the first plasma concentration of the protein.

Embodiment 143. The method of embodiment 142, further comprising administering a second therapeutically-effective amount of the compound.

Embodiment 144. The method of embodiment 136, wherein the second plasma concentration of the protein is lower than the first plasma concentration of the protein.

Embodiment 145. The method of embodiment 144, further comprising administering a second therapeutically-effective amount of the compound.

Embodiment 146. The method of any one of embodiments 132-145, wherein the compound is of the formula:

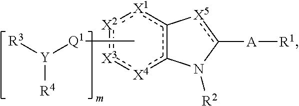

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

Embodiment 147. The method of embodiment 146, wherein A is alkylene, alkenylene, or alkynylene, each of which is substituted or unsubstituted.

Embodiment 148. The method of embodiment 146, wherein A is aryl, heteroaryl, or heterocyclyl, each of which is substituted or unsubstituted.

Embodiment 149. The method of embodiment 146 or 147, wherein the compound is of the formula:

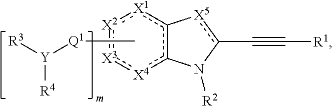

Embodiment 150. The method of embodiment 149, wherein Q¹ is C₁-alkylene.

Embodiment 151. The method of embodiment 149, wherein Q¹ is a bond.

Embodiment 152. The method of any one of embodiments 149-151, wherein m is 1.

Embodiment 153. The method of any one of embodiments 149-151, wherein m is 2.

Embodiment 154. The method of any one of embodiments 149-153, wherein Y is N.

Embodiment 155. The method of any one of embodiments 149-153, wherein Y is O.

Embodiment 156. The method of any one of embodiments 149-155, wherein each R³ and R⁴ is independently alkyl, alkylene, alkenyl, alkenylene, alkynyl, aryl, heteroaryl, or heterocyclyl, each of which is independently substituted or unsubstituted; or hydrogen.

Embodiment 157. The method of embodiment 156, wherein R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted.

Embodiment 158. The method of embodiment 156, wherein R³ is H; and R⁴ is aryl, heteroaryl, or heterocyclyl, each of which is independently substituted or unsubstituted.

Embodiment 159. The method of any one of embodiments 149-158, wherein R¹³ is hydrogen.

Embodiment 160. The method of any one of embodiments 149, 151, 152, 154 and 156-159, wherein the compound is of the formula:

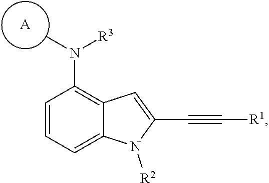

wherein ring A is a cyclic group that is substituted or unsubstituted.

Embodiment 161. The method of embodiment 160, wherein R² is substituted or unsubstituted alkyl.

Embodiment 162. The method of embodiment 160 or 161, wherein R² is methyl, ethyl, propyl, iso-propyl, butyl, or tert-butyl, each of which is substituted or unsubstituted.

Embodiment 163. The method of any one of embodiments 160-162, wherein R² is substituted ethyl.

Embodiment 164. The method of embodiment 163, wherein R² is trifluoroethyl.

Embodiment 165. The method of any one of embodiments 160-164, wherein the compound is of the formula:

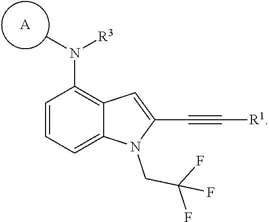

Embodiment 166. The method of embodiment 165, wherein ring A is aryl, heteroaryl, or heterocyclyl, each of which is substituted or unsubstituted.

Embodiment 167. The method of embodiment 166 or 167, wherein ring A is substituted aryl.

Embodiment 168. The method of embodiment 166 or 167, wherein ring A is substituted heteroaryl.

Embodiment 169. The method of embodiment 166 or 167, wherein ring A is substituted heterocyclyl.

Embodiment 170. The method of any one of embodiments 165-169, wherein R¹ is alkyl, alkenyl, —C(O)R¹⁶, —C(O)OR¹⁶, or —C(O)NR¹⁶R¹⁷, each of which is unsubstituted or substituted.

Embodiment 171. The method of embodiment 170, wherein R¹ is substituted alkyl.

Embodiment 172. The method of embodiment 170 or 171, wherein R¹ is alkyl substituted with NR¹⁶R¹⁷.

Embodiment 173. The method of embodiment 172, wherein the compound is of the formula:

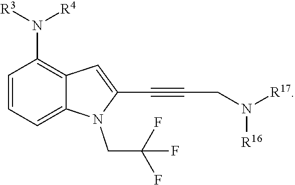

Embodiment 174. The method of embodiment 172 or 173, wherein each R¹⁶ and R¹⁷ is independently alkyl, alkenyl, aryl, heteroaryl, heterocyclyl, each of which is independently substituted or unsubstituted; or hydrogen.

Embodiment 175. The method of any one of embodiments 172-174, wherein R¹⁶ is hydrogen or alkyl.

Embodiment 176. The method of any one of embodiments 172-174, wherein R¹⁷ is aryl, heteroaryl, or heterocyclyl, each of which is independently substituted or unsubstituted.

Embodiment 177. The method of embodiment 176, wherein R¹⁷ is substituted aryl.

Embodiment 178. The method of embodiment 176 or 177, wherein R¹⁷ is substituted phenyl.

Embodiment 179. The method of any one of embodiments 176-178, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.

Embodiment 180. The method of any one of embodiments 176-179, wherein R¹⁷ is phenyl substituted with methoxy.

Embodiment 181. The method of any one of embodiments 176-179, wherein R¹⁷ is phenyl substituted with a substituted sulfoxide group.

Embodiment 182. The method of any one of embodiments 176-179, wherein R¹⁷ is phenyl substituted with a carboxyl group.

Embodiment 183. The method of any one of embodiments 176-179, wherein R¹⁷ is phenyl substituted with an amide group.

Embodiment 184. The method of embodiment 146, wherein the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide.

Embodiment 185. The method of embodiment 146, wherein the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine.

Embodiment 186. The method of embodiment 146, wherein the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide.

Embodiment 187. The method of embodiment 146, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide.

Embodiment 188. The method of embodiment 146, wherein the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide.

Embodiment 189. The method of embodiment 146, wherein the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide.

Embodiment 190. The method of embodiment 146, wherein the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide.

Embodiment 191. The method of embodiment 146, wherein the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide.

Embodiment 192. The method of embodiment 146, wherein the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

Embodiment 193. A method of treating a cancer, the method comprising administering to a subject in need thereof a therapeutically-effective amount of a compound that binds to a mutant p53 protein in the subject and reconforms the mutant p53 protein to a conformation of p53 that exhibits anti-cancer activity within about 2 hours of contacting the cancer with the compound.

Embodiment 194. The method of embodiment 193, wherein the conformation of p53 that exhibits anti-cancer activity is sustained for at least about 8 hours.

Embodiment 195. The method of embodiment 193, wherein the conformation of p53 that exhibits anti-cancer activity is sustained for at least about 16 hours.

Embodiment 196. The method of embodiment 193, wherein the conformation of p53 that exhibits anti-cancer activity is sustained for at least about 24 hours.

Embodiment 197. The method of any one of embodiments 193-196, wherein the compound is of the formula:

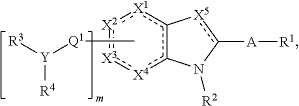

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

Embodiment 198. The method of embodiment 197, wherein A is alkylene, alkenylene, or alkynylene, each of which is substituted or unsubstituted.

Embodiment 199. The method of embodiment 197, wherein A is aryl, heteroaryl, or heterocyclyl, each of which is substituted or unsubstituted.

Embodiment 200. The method of embodiment 197 or 198, wherein the compound is of the formula:

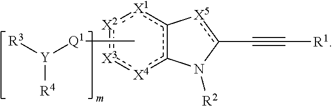

Embodiment 201. The method of embodiment 200, wherein Q¹ is C₁-alkylene.

Embodiment 202. The method of embodiment 200, wherein Q¹ is a bond.

Embodiment 203. The method of any one of embodiments 200-202, wherein m is 1.

Embodiment 204. The method of any one of embodiments 200-202, wherein m is 2.

Embodiment 205. The method of any one of embodiments 200-204, wherein Y is N.

Embodiment 206. The method of any one of embodiments 200-204, wherein Y is O.

Embodiment 207. The method of any one of embodiments 200-206, wherein each R³ and R⁴ is independently alkyl, alkylene, alkenyl, alkenylene, alkynyl, aryl, heteroaryl, or heterocyclyl, each of which is independently substituted or unsubstituted; or hydrogen.

Embodiment 208. The method of embodiment 207, wherein R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted.

Embodiment 209. The method of embodiment 207, wherein R³ is H; and R⁴ is aryl, heteroaryl, or heterocyclyl, each of which is independently substituted or unsubstituted.

Embodiment 210. The method of any one of embodiments 200-209, wherein R¹³ is hydrogen.

Embodiment 211. The method of any one of embodiments 200, 202, 203, 205 and 207-210, wherein the compound is of the formula:

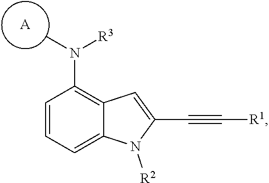

wherein ring A is a cyclic group that is substituted or unsubstituted.

Embodiment 212. The method of embodiment 211, wherein R² is substituted or unsubstituted alkyl.

Embodiment 213. The method of embodiment 212 or 213, wherein R² is methyl, ethyl, propyl, iso-propyl, butyl, or tert-butyl, each of which is substituted or unsubstituted.

Embodiment 214. The method of any one of embodiments 211-213, wherein R² is substituted ethyl.

Embodiment 215. The method of embodiment 214, wherein R² is trifluoroethyl.

Embodiment 216. The method of any one of embodiments 211-215, wherein the compound is of the formula:

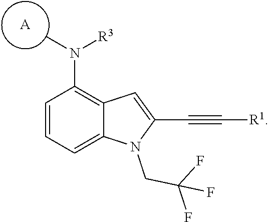

Embodiment 217. The method of embodiment 216, wherein ring A is aryl, heteroaryl, or heterocyclyl, each of which is substituted or unsubstituted.

Embodiment 218. The method of embodiment 216 or 217, wherein ring A is substituted aryl.

Embodiment 219. The method of embodiment 216 or 217, wherein ring A is substituted heteroaryl.

Embodiment 220. The method of embodiment 216 or 217, wherein ring A is substituted heterocyclyl.

Embodiment 221. The method of any one of embodiments 216-220, wherein R¹ is alkyl, alkenyl, —C(O)R¹⁶, —C(O)OR¹⁶, or —C(O)NR¹⁶R¹⁷, each of which is unsubstituted or substituted.

Embodiment 222. The method of embodiment 221, wherein R¹ is substituted alkyl.

Embodiment 223. The method of embodiment 221 or 222, wherein R¹ is alkyl substituted with NR¹⁶R¹⁷.

Embodiment 224. The method of embodiment 223, wherein the compound is of the formula:

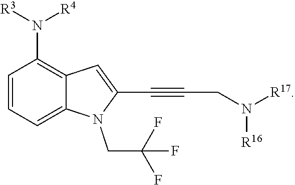

Embodiment 225. The method of embodiment 223 or 224, wherein each R¹⁶ and R¹⁷ is independently alkyl, alkenyl, aryl, heteroaryl, heterocyclyl, each of which is independently substituted or unsubstituted; or hydrogen.

Embodiment 226. The method of any one of embodiments 223-225, wherein R¹⁶ is hydrogen or alkyl.

Embodiment 227. The method of any one of embodiments 223-225, wherein R¹⁷ is aryl, heteroaryl, or heterocyclyl, each of which is independently substituted or unsubstituted.

Embodiment 228. The method of embodiment 227, wherein R¹⁷ is substituted aryl.

Embodiment 229. The method of embodiment 227 or 228, wherein R¹⁷ is substituted phenyl.

Embodiment 230. The method of any one of embodiments 227-229, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.

Embodiment 231. The method of any one of embodiments 227-230, wherein R¹⁷ is phenyl substituted with methoxy.

Embodiment 232. The method of any one of embodiments 227-230, wherein R¹⁷ is phenyl substituted with a substituted sulfoxide group.

Embodiment 233. The method of any one of embodiments 227-230, wherein R¹⁷ is phenyl substituted with a carboxyl group.

Embodiment 234. The method of any one of embodiments 227-230, wherein R¹⁷ is phenyl substituted with an amide group.

Embodiment 235. The method of embodiment 197, wherein the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide.

Embodiment 236. The method of embodiment 197, wherein the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine.

Embodiment 237. The method of embodiment 197, wherein the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide.

Embodiment 238. The method of embodiment 197, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide.

Embodiment 239. The method of embodiment 197, wherein the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide.

Embodiment 240. The method of embodiment 197, wherein the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide.

Embodiment 241. The method of embodiment 197, wherein the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide.

Embodiment 242. The method of embodiment 197, wherein the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide.

Embodiment 243. The method of embodiment 197, wherein the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

Embodiment 244. A method of treating a cancer, the method comprising administering to a subject in need thereof a therapeutically-effective amount of a compound that binds to a mutant p53 protein and reconforms the mutant p53 protein to a conformation of p53 that exhibits anti-cancer activity, wherein the cancer is heterozygous for a p53 Y220C mutation.

Embodiment 245. The method of embodiment 244, wherein the cancer is uterine cancer.

Embodiment 246. The method of embodiment 245, wherein the uterine cancer is endometrial adenocarcinoma.

Embodiment 247. The method of embodiment 244, wherein the cancer is breast cancer.

Embodiment 248. The method of embodiment 247, wherein the breast cancer is breast ductal carcinoma.

Embodiment 249. The method of any one of embodiments 244-248, wherein the compound is of the formula:

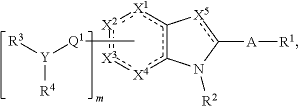

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

Embodiment 250. The method of embodiment 249, wherein A is alkylene, alkenylene, or alkynylene, each of which is substituted or unsubstituted.

Embodiment 251. The method of embodiment 249, wherein A is aryl, heteroaryl, or heterocyclyl, each of which is substituted or unsubstituted.

Embodiment 252. The method of embodiment 249 or 250, wherein the compound is of the formula:

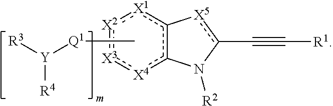

Embodiment 253. The method of embodiment 252, wherein Q¹ is C₁-alkylene.

Embodiment 254. he method of embodiment 252, wherein Q¹ is a bond.

Embodiment 255. The method of any one of embodiments 252-254, wherein m is 1.

Embodiment 256. The method of any one of embodiments 252-254, wherein m is 2.

Embodiment 257. The method of any one of embodiments 252-256, wherein Y is N.

Embodiment 258. The method of any one of embodiments 252-256, wherein Y is O.

Embodiment 259. The method of any one of embodiments 252-258, wherein each R³ and R⁴ is independently alkyl, alkylene, alkenyl, alkenylene, alkynyl, aryl, heteroaryl, or heterocyclyl, each of which is independently substituted or unsubstituted; or hydrogen.

Embodiment 260. The method of embodiment 259, wherein R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted.

Embodiment 261. The method of embodiment 259, wherein R³ is H; and R⁴ is aryl, heteroaryl, or heterocyclyl, each of which is independently substituted or unsubstituted.

Embodiment 262. The method of any one of embodiments 252-261, wherein R¹³ is hydrogen.

Embodiment 263. The method of any one of embodiments 252, 254, 255, 257 and 259-262, wherein the compound is of the formula:

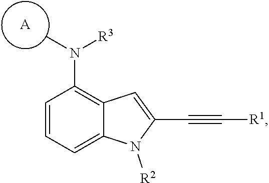

wherein ring A is a cyclic group that is substituted or unsubstituted.

Embodiment 264. The method of embodiment 263, wherein R² is substituted or unsubstituted alkyl.

Embodiment 265. The method of embodiment 263 or 264, wherein R² is methyl, ethyl, propyl, iso-propyl, butyl, or tert-butyl, each of which is substituted or unsubstituted.

Embodiment 266. The method of any one of embodiments 263-265, wherein R² is substituted ethyl.

Embodiment 267. The method of embodiment 266, wherein R² is trifluoroethyl.

Embodiment 268. The method of any one of embodiments 263-267, wherein the compound is of the formula:

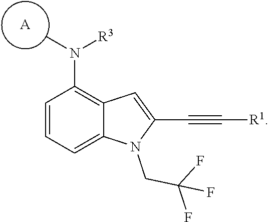

Embodiment 269. The method of embodiment 268, wherein ring A is aryl, heteroaryl, or heterocyclyl, each of which is substituted or unsubstituted.

Embodiment 270. The method of embodiment 268 or 269, wherein ring A is substituted aryl.

Embodiment 271. The method of embodiment 268 or 269, wherein ring A is substituted heteroaryl.

Embodiment 272. The method of embodiment 268 or 269, wherein ring A is substituted heterocyclyl.

Embodiment 273. The method of any one of embodiments 268-272, wherein R¹ is alkyl, alkenyl, —C(O)R¹⁶, —C(O)OR¹⁶, or —C(O)NR¹⁶R¹⁷, each of which is unsubstituted or substituted.

Embodiment 274. The method of embodiment 273, wherein R¹ is substituted alkyl.

Embodiment 275. The method of embodiment 273 or 274, wherein R¹ is alkyl substituted with NR¹⁶R¹⁷.

Embodiment 276. The method of embodiment 275, wherein the compound is of the formula:

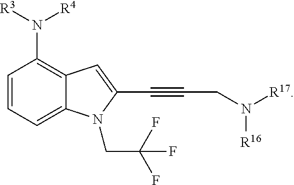

Embodiment 277. The method of embodiment 275 or 276, wherein each R¹⁶ and R¹⁷ is independently alkyl, alkenyl, aryl, heteroaryl, heterocyclyl, each of which is independently substituted or unsubstituted; or hydrogen.

Embodiment 278. The method of any one of embodiments 275-277, wherein R¹⁶ is hydrogen or alkyl.

Embodiment 279. The method of any one of embodiments 275-277, wherein R¹⁷ is aryl, heteroaryl, or heterocyclyl, each of which is independently substituted or unsubstituted.

Embodiment 280. The method of embodiment 279, wherein R¹⁷ is substituted aryl.

Embodiment 281. The method of embodiment 279 or 280, wherein R¹⁷ is substituted phenyl.

Embodiment 282. The method of any one of embodiments 279-281, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.

Embodiment 283. The method of any one of embodiments 279-282, wherein R¹⁷ is phenyl substituted with methoxy.

Embodiment 284. The method of any one of embodiments 279-282, wherein R¹⁷ is phenyl substituted with a substituted sulfoxide group.

Embodiment 285. The method of any one of embodiments 279-282, wherein R¹⁷ is phenyl substituted with a carboxyl group.

Embodiment 286. The method of any one of embodiments 279-282, wherein R¹⁷ is phenyl substituted with an amide group.

Embodiment 287. The method of embodiment 249, wherein the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide.

Embodiment 288. The method of embodiment 249, wherein the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine.

Embodiment 289. The method of embodiment 249, wherein the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide.

Embodiment 290. The method of embodiment 249, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide.

Embodiment 291. The method of embodiment 249, wherein the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide.

Embodiment 292. The method of embodiment 249, wherein the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide.

Embodiment 293. The method of embodiment 249, wherein the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide.

Embodiment 294. The method of embodiment 249, wherein the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide.

Embodiment 295. The method of embodiment 249, wherein the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

Embodiment 296. A method of treating a cancer, the method comprising administering to a subject in need thereof a therapeutically-effective amount of a compound that binds a mutant p53 protein in the subject, wherein binding of the compound to the mutant p53 protein in the subject modulates at least two genes downstream of p53 in the subject, wherein the genes are APAF1, BAX, BBC3, BIRC5, BRCA2, BRCA1, BTG2, CCNB1, CCNE1, CCNG1, CDC25A, CDC25C, CDK1, CDKN1A, CHEK1, CHEK2, E2F1, EGR1, FAS, GADD45A, GAPDH, GDF15, IL6, MDM2, MSH2, p21, PIDD1, PPM1D, PRC1, SESN2, TNFRSF10B, TNFRSF10D, and TP53.

Embodiment 297. The method of embodiment 296, wherein the compound modulates two genes.

Embodiment 298. The method of embodiment 296, wherein the compound modulates three genes.

Embodiment 299. The method of embodiment 296, wherein the compound modulates four genes.

Embodiment 300. The method of embodiment 296, wherein the compound modulates five genes.

Embodiment 301. The method of embodiment 296, wherein the at least two genes comprises p21.

Embodiment 302. The method of embodiment 296, wherein the at least two genes comprises MDM2.

Embodiment 303. The method of embodiment 296, wherein the at least two genes comprises GDF15.

Embodiment 304. The method of embodiment 296, wherein the at least two genes comprises GAPDH.

Embodiment 305. The method of any one of embodiments 296-304, wherein the compound is of the formula:

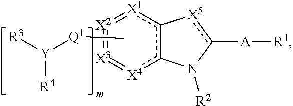

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

Embodiment 306. The method of embodiment 305, wherein A is alkylene, alkenylene, or alkynylene, each of which is substituted or unsubstituted.

Embodiment 307. The method of embodiment 305, wherein A is aryl, heteroaryl, or heterocyclyl, each of which is substituted or unsubstituted.

Embodiment 308. The method of embodiment 305 or 306, wherein the compound is of the formula:

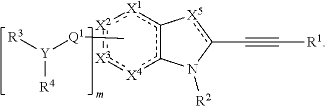

Embodiment 309. The method of embodiment 308, wherein Q¹ is C₁-alkylene.

Embodiment 310. The method of embodiment 308, wherein Q¹ is a bond.

Embodiment 311. The method of any one of embodiments 308-310, wherein m is 1.

Embodiment 312. The method of any one of embodiments 308-310, wherein m is 2.

Embodiment 313. The method of any one of embodiments 308-312, wherein Y is N.

Embodiment 314. The method of any one of embodiments 308-312, wherein Y is O.

Embodiment 315. The method of any one of embodiments 308-314, wherein each R³ and R⁴ is independently alkyl, alkylene, alkenyl, alkenylene, alkynyl, aryl, heteroaryl, or heterocyclyl, each of which is independently substituted or unsubstituted; or hydrogen.

Embodiment 316. The method of embodiment 315, wherein R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted.

Embodiment 317. The method of embodiment 315, wherein R³ is H; and R⁴ is aryl, heteroaryl, or heterocyclyl, each of which is independently substituted or unsubstituted.

Embodiment 318. The method of any one of embodiments 308-317, wherein R¹³ is hydrogen.

Embodiment 319. The method of embodiment 308, 310, 311, 313 and 315-318 wherein the compound is of the formula:

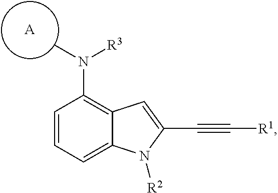

wherein ring A is a cyclic group that is substituted or unsubstituted.

Embodiment 320. The method of embodiment 319, wherein R² is substituted or unsubstituted alkyl.

Embodiment 321. The method of embodiment 319 or 320, wherein R² is methyl, ethyl, propyl, iso-propyl, butyl, or tert-butyl, each of which is substituted or unsubstituted.

Embodiment 322. The method of any one of embodiments 319-321, wherein R² is substituted ethyl.

Embodiment 323. The method of embodiment 322, wherein R² is trifluoroethyl.

Embodiment 324. The method of any one of embodiments 319-323, wherein the compound is of the formula:

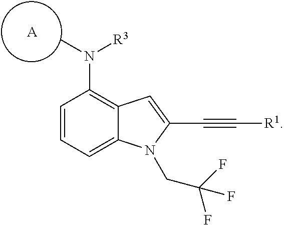

Embodiment 325. The method of embodiment 324, wherein ring A is aryl, heteroaryl, or heterocyclyl, each of which is substituted or unsubstituted.

Embodiment 326. The method of embodiment 324 or 325, wherein ring A is substituted aryl.

Embodiment 327. The method of embodiment 324 or 325, wherein ring A is substituted heteroaryl.

Embodiment 328. The method of embodiment 324 or 325, wherein ring A is substituted heterocyclyl.

Embodiment 329. The method of any one of embodiments 324-328, wherein R¹ is alkyl, alkenyl, —C(O)R¹⁶, —C(O)OR¹⁶, or —C(O)NR¹⁶R¹⁷, each of which is unsubstituted or substituted.

Embodiment 330. The method of embodiment 329, wherein R¹ is substituted alkyl.

Embodiment 331. The method of embodiment 329 or 330, wherein R¹ is alkyl substituted with NR¹⁶R¹⁷.

Embodiment 332. The method of embodiment 331, wherein the compound is of the formula:

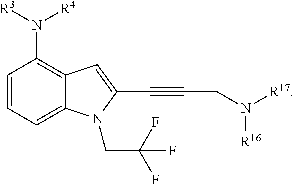

Embodiment 333. The method of embodiment 331 or 332, wherein each R¹⁶ and R¹⁷ is independently alkyl, alkenyl, aryl, heteroaryl, heterocyclyl, each of which is independently substituted or unsubstituted; or hydrogen.

Embodiment 334. The method of any one of embodiments 331-333, wherein R¹⁶ is hydrogen or alkyl.

Embodiment 335. The method of any one of embodiments 331-333, wherein R¹⁷ is aryl, heteroaryl, or heterocyclyl, each of which is independently substituted or unsubstituted.

Embodiment 336. The method of embodiment 335, wherein R¹⁷ is substituted aryl.

Embodiment 337. The method of embodiment 335 or 336, wherein R¹⁷ is substituted phenyl.

Embodiment 338. The method of any one of embodiments 335-337, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.

Embodiment 339. The method of any one of embodiments 335-338, wherein R¹⁷ is phenyl substituted with methoxy.

Embodiment 340. The method of any one of embodiments 335-338, wherein R¹⁷ is phenyl substituted with a substituted sulfoxide group.

Embodiment 341 The method of any one of embodiments 335-338, wherein R¹⁷ is phenyl substituted with a carboxyl group.

Embodiment 342. The method of any one of embodiments 335-338, wherein R¹⁷ is phenyl substituted with an amide group.

Embodiment 343. The method of embodiment 305, wherein the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide.

Embodiment 344. The method of embodiment 305, wherein the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine.

Embodiment 345. The method of embodiment 305, wherein the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide.

Embodiment 346. The method of embodiment 305, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide.

Embodiment 347. The method of embodiment 305, wherein the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide.

Embodiment 348. The method of embodiment 305, wherein the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide.

Embodiment 349. The method of embodiment 305, wherein the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide.

Embodiment 350. The method of embodiment 305, wherein the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide.

Embodiment 351. The method of embodiment 305, wherein the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

Embodiment 352. A compound comprising a structure that binds to a mutant p53 protein and increases wild type p53 activity of the mutant p53 protein; wherein if in a controlled study, a therapeutically-effective amount of the compound is administered to a first subject with a cancer that expresses mutant p53, then a plasma concentration in the first subject of a protein that is a biomarker of wild-type p53 activity when measured from about 8 to about 72 hours after administration of the compound is determined to be at least about 2-fold greater than that determined in a second subject who was not administered the compound, as determined by an enzyme-linked immunosorbent assay.

Embodiment 353. The compound of embodiment 352, wherein the plasma concentration of the first subject is at least about 5-fold greater than the plasma concentration of the second subject.

Embodiment 354. The compound of embodiment 352, wherein the plasma concentration of the first subject is at least about 8-fold greater than the plasma concentration of the second subject.

Embodiment 355. The compound of embodiment 352, wherein the plasma concentration of the first subject is at least about 20-fold greater than the plasma concentration of the second subject.

Embodiment 356. The compound of embodiment 352, wherein the plasma concentration of the first subject is at least about 40-fold greater than the plasma concentration of the second subject.

Embodiment 357. The compound of any one of embodiments 352-356, wherein the structure comprises a substituted heterocyclyl group.

Embodiment 358. The compound of any one of embodiments 352-357, wherein the structure comprises a heterocyclyl group comprising a halo substituent.

Embodiment 359. The compound of any one of embodiments 352-358, wherein the structure comprises an indole group.

Embodiment 360. The compound of embodiment 359, wherein the indole group comprises a propargyl substituent at a 2-position of the indole group.

Embodiment 361. The compound of embodiment 360, wherein the propargyl substituent is attached to the indole group via an sp carbon atom of the propargyl substituent.

Embodiment 362. The compound of embodiment 360, wherein the propargyl substituent is attached to a nitrogen atom of an aniline group via a methylene group of the propargyl substituent.

Embodiment 363. The compound of embodiment 360, wherein the indole group comprises an amino substituent at a 4-position of the indole group.

Embodiment 364. The compound of embodiment 363, wherein the amino substituent is attached to the heterocyclyl group.

Embodiment 365. The compound of any one of embodiments 352-364, wherein the compound is of the formula:

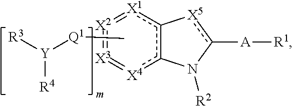

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

Embodiment 366. The method of embodiment 365, wherein A is alkylene, alkenylene, or alkynylene, each of which is substituted or unsubstituted.

Embodiment 367. The method of embodiment 365, wherein A is aryl, heteroaryl, or heterocyclyl, each of which is substituted or unsubstituted.

Embodiment 368. The method of embodiment 365 or 366, wherein the compound is of the formula:

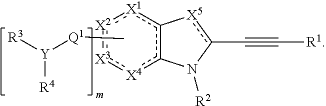

Embodiment 369. The method of embodiment 368, wherein Q¹ is C₁-alkylene.

Embodiment 370. The method of embodiment 368, wherein Q¹ is a bond.

Embodiment 371. The method of any one of embodiments 368-370, wherein m is 1.

Embodiment 372. The method of any one of embodiments 368-370, wherein m is 2.

Embodiment 373. The method of any one of embodiments 368-372, wherein Y is N.

Embodiment 374. The method of any one of embodiments 368-372, wherein Y is O.

Embodiment 375. The method of any one of embodiments 368-374, wherein each R³ and R⁴ is independently alkyl, alkylene, alkenyl, alkenylene, alkynyl, aryl, heteroaryl, or heterocyclyl, each of which is independently substituted or unsubstituted; or hydrogen.

Embodiment 376. The method of embodiment 375, wherein R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted.

Embodiment 377. The method of embodiment 375, wherein R³ is H; and R⁴ is aryl, heteroaryl, or heterocyclyl, each of which is independently substituted or unsubstituted.

Embodiment 378. The method of any one of embodiments 368-377, wherein R¹³ is hydrogen.

Embodiment 379. The method of any one of embodiments 368, 370, 371, 373 and 375-378, wherein the compound is of the formula:

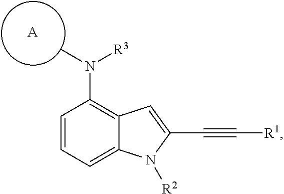

wherein ring A is a cyclic group that is substituted or unsubstituted.

Embodiment 380. The method of embodiment 379, wherein R² is substituted or unsubstituted alkyl.

Embodiment 381. The method of embodiment 379 or 380, wherein R² is methyl, ethyl, propyl, iso-propyl, butyl, or tert-butyl, each of which is substituted or unsubstituted.

Embodiment 382. The method of any one of embodiments 379-381, wherein R² is substituted ethyl.

Embodiment 383. The method of embodiment 382, wherein R² is trifluoroethyl.

Embodiment 384. The method of any one of embodiments 379-383, wherein the compound is of the formula:

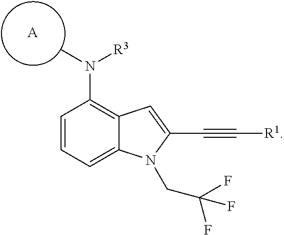

Embodiment 385. The method of embodiment 384, wherein ring A is aryl, heteroaryl, or heterocyclyl, each of which is substituted or unsubstituted.

Embodiment 386. The method of embodiment 384 or 385, wherein ring A is substituted aryl.

Embodiment 387. The method of embodiment 384 or 385, wherein ring A is substituted heteroaryl.

Embodiment 388. The method of embodiment 384 or 385, wherein ring A is substituted heterocyclyl.

Embodiment 389. The method of any one of embodiments 384-388, wherein R¹ is alkyl, alkenyl, —C(O)R¹⁶, —C(O)OR¹⁶, or —C(O)NR¹⁶R¹⁷, each of which is unsubstituted or substituted.

Embodiment 390. The method of embodiment 389, wherein R¹ is substituted alkyl.

Embodiment 391. The method of embodiment 389 or 390, wherein R¹ is alkyl substituted with NR¹⁶R¹⁷.

Embodiment 392. The method of embodiment 391, wherein the compound is of the formula:

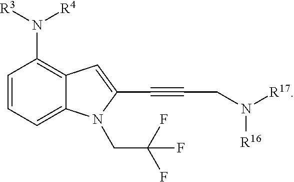

Embodiment 393. The method of embodiment 391 or 392, wherein each R¹⁶ and R¹⁷ is independently alkyl, alkenyl, aryl, heteroaryl, heterocyclyl, each of which is independently substituted or unsubstituted; or hydrogen.

Embodiment 394. The method of any one of embodiments 391-393, wherein R¹⁶ is hydrogen or alkyl.

Embodiment 395. The method of any one of embodiments 391-393, wherein R¹⁷ is aryl, heteroaryl, or heterocyclyl, each of which is independently substituted or unsubstituted.

Embodiment 396. The method of embodiment 395, wherein R¹⁷ is substituted aryl.

Embodiment 397. The method of embodiment 395 or 396, wherein R¹⁷ is substituted phenyl.

Embodiment 398. The method of any one of embodiments 395-397, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.

Embodiment 399. The method of any one of embodiments 395-398, wherein R¹⁷ is phenyl substituted with methoxy.

Embodiment 400. The method of any one of embodiments 395-398, wherein R¹⁷ is phenyl substituted with a substituted sulfoxide group.

Embodiment 401. The method of any one of embodiments 395-398, wherein R¹⁷ is phenyl substituted with a carboxyl group.

Embodiment 402. The method of any one of embodiments 395-398, wherein R¹⁷ is phenyl substituted with an amide group.

Embodiment 403. The method of embodiment 365, wherein the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide.

Embodiment 404. The method of embodiment 365, wherein the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine.

Embodiment 405. The method of embodiment 365, wherein the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide.

Embodiment 406. The method of embodiment 365, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide.

Embodiment 407. The method of embodiment 365, wherein the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide.

Embodiment 408. The method of embodiment 365, wherein the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide.

Embodiment 409. The method of embodiment 365, wherein the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide.

Embodiment 410. The method of embodiment 365, wherein the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide.

Embodiment 411. The method of embodiment 365, wherein the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

1. A method of treating a cancer, the method comprising administering to a subject in need thereof a therapeutically-effective amount of a compound that binds to a mutant p53 protein and reconforms the mutant p53 protein to a conformation of p53 that exhibits anti-cancer activity, wherein the mutant p53 protein comprises a mutation at Y220C, wherein the compound has a half-maximal inhibitory concentration (IC₅₀) in a cancer cell that has a Y220C mutant p53 protein that is at least about 2-fold lesser than in a cancer cell that does not have any Y220C mutant p53 protein.

2. The method of claim 1, wherein the therapeutically-effective amount is from about 500 mg to about 2000 mg/kg.

3-4. (canceled)

5. The method of claim 1, wherein the compound selectively binds the mutant p53 protein compared to a wild type p53 protein.

6. The method of claim 1, wherein the conformation of p53 that exhibits anti-cancer activity is a wild type conformation p53 protein.

7. The method of claim 1, wherein the IC₅₀ of the compound is less than about 5 μM.

8-11. (canceled)

12. The method of claim 1, wherein the cancer is gastric cancer.

13. (canceled)

14. The method of claim 1, wherein the administering is oral.

15. The method of claim 1, wherein the subject is human.

16. The method of claim 1, wherein the compound is of the formula: 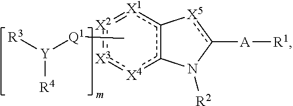
wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, heterocyclyl, or halo, each of which is independently substituted or unsubstituted, or hydrogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
or a pharmaceutically-acceptable salt thereof.

17. The method of claim 16, wherein A is alkylene, alkenylene, or alkynylene, each of which is substituted or unsubstituted.

18. The method of claim 17, wherein the compound is of the formula:

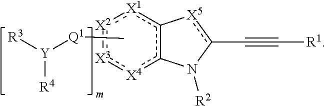

19-23. (canceled)

24. The method of claim 18, wherein R³ is H; and R⁴ is aryl, heteroaryl, or heterocyclyl, each of which is independently substituted or unsubstituted.

25. The method of claim 18, wherein R¹³ is hydrogen.

26. The method of claim 18, wherein the compound is of the formula: 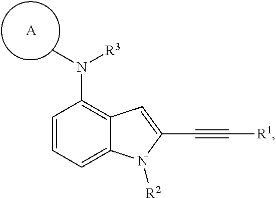
wherein ring A is a cyclic group that is substituted or unsubstituted.

27-28. (canceled)

29. The method of claim 26, wherein R² is trifluoroethyl.

30. The method of claim 26, wherein ring A is aryl, heteroaryl, or heterocyclyl, each of which is substituted or unsubstituted.

31-32. (canceled)

33. The method of claim 26, wherein R¹ is alkyl, alkenyl, —C(O)R¹⁶, —C(O)OR¹⁶, or —C(O)NR¹⁶R¹⁷, each of which is unsubstituted or substituted.

34. (canceled)

35. The method of claim 26, wherein the compound is of the formula:

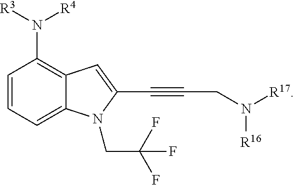

36. The method of claim 35, wherein each R¹⁶ and R¹⁷ is independently alkyl, alkenyl, aryl, heteroaryl, heterocyclyl, each of which is independently substituted or unsubstituted; or hydrogen.

37. The method of claim 36, wherein R¹⁶ is hydrogen or alkyl.

38-39. (canceled)

40. The method of claim 36, wherein R¹⁷ is phenyl substituted with a sulfoxide group, carboxyl group, amide group, amino group, alkyl, alkoxy, hydroxy, halo, cyano, or heterocyclyl, each of which is independently substituted or unsubstituted.
